(19) United States
(12) Patent Application Publication
(10) Pub. No.: US 2024/0254494 A1
(43) Pub. Date: Aug. 1, 2024

(54) GENE EDITING SYSTEMS COMPRISING AN RNA GUIDE TARGETING HYDROXYACID OXIDASE 1 (HAO1) AND USES THEREOF

(71) Applicant: Arbor Biotechnologies, Inc., Cambridge, MA (US)

(72) Inventors: Quinton Norman Wessells, Cambridge, MA (US); Jeffrey Raymond Haswell, Needham, MA (US); Tia Marie Ditommaso, Newton, MA (US); Noah Michael Jakimo, Cambridge, MA (US); Sejuti Sengupta, Ashland, MA (US)

(73) Assignee: Arbor Biotechnologies, Inc., Cambridge, MA (US)

(21) Appl. No.: 18/565,575

(22) PCT Filed: Jun. 3, 2022
(86) PCT No.: PCT/US2022/032144

§ 371 Date: Nov. 30, 2023

Related U.S. Application Data
(60) Provisional application No. 63/197,073, filed on Jun. 4, 2021.
(60) Provisional application No. 63/225,046, filed on Jul. 23, 2021.
(60) Provisional application No. 63/292,889, filed on Dec. 22, 2021.
(60) Provisional application No. 63/300,727, filed on Jan. 19, 2022.

(51) Int. Cl. C12N 15/113; C12N 9/22; C12N 15/86; C12N 15/88; C12N 15/90

(52) U.S. Cl. CPC C12N 15/1137; C12N 9/22; C12N 15/86; C12N 15/88; C12N 15/907; C12Y 101/03015; C12N 2310/20; C12N 2750/14143; C12N 2800/80

(57) ABSTRACT

Provided herein are gene editing systems and/or compositions comprising RNA guides targeting HAO1 for use in genetic editing of the HAO1 gene. Also provide herein are methods of using the gene editing system for introducing edits to the HAO1 gene and/or for treatment of primary hyperoxaluria (PH), and processes for characterizing the gene editing system.

### CROSS REFERENCE TO RELATED APPLICATIONS

This application claims the benefit under 35 U.S.C. § 119(c) of U.S. Provisional Application No. 63/197,073, filed Jun. 4, 2021, U.S. Provisional Application No. 63/225,046, filed Jul. 23, 2021, U.S. Provisional Application No. 63/292,889, filed Dec. 22, 2021, and U.S. Provisional Application No. 63/300,727, filed Jan. 19, 2022, the contents of each of which are incorporated by reference herein in their entirety.

### SEQUENCE LISTING

The instant application contains a Sequence Listing which has been filed electronically in ASCII format and is hereby incorporated by reference in its entirety. Said ASCII copy, created on Jun. 3, 2022, is named 116928-0040-0004WO00_SEQ.txt and is 367,354 bytes in size.

### BACKGROUND

Clustered Regularly Interspaced Short Palindromic Repeats (CRISPR) and CRISPR-associated (Cas) genes, collectively known as CRISPR-Cas or CRISPR/Cas systems, are adaptive immune systems in archaea and bacteria that defend particular species against foreign genetic elements.

### SUMMARY OF THE INVENTION

The present disclosure is based, at least in part, on the development of a system for genetic editing of a hydroxyacid oxidase 1 (HAO1) gene. The system involves a Cas12i CRISPR nuclease polypeptide (e.g., a Cas12i2 polypeptide) and an RNA guide mediating cleavage at a genetic site within the HAO1 gene by the CRISPR nuclease polypeptide. As reported herein, the gene editing system disclosed herein has achieved successful editing of HAO1 gene with high editing efficiency and accuracy.

Without being bound by theory, the gene editing system disclosed herein may further exhibit one or more of the following advantageous features. Compared to SpCas9 and Cas12a. Cas12i effectors are smaller (1033 to 1093aa), which, in conjunction with their short mature crRNA (40-43 nt), is preferable in terms of delivery and cost of synthesis. Cas12i cleavage results in larger deletions compared to the small deletions and +1 insertions induced by Cas9 cleavage. Cas12i PAM sequences also differ from those of Cas9. Therefore, larger and different portions of genetic sites of interest can be disrupted with a Cas12i polypeptide and RNA guide compared to Cas9. Using an unbiased approach of tagmentation-based tag integration site sequencing (TTISS), more potential off-target sites with a higher number of unique integration events were identified for SpCas9 compared to Cas12i2. See WO/2021/202800. Therefore, Cas12i such as Cas12i2 may be more specific than Cas9.

Accordingly, provided herein are gene editing systems for editing HAO1 gene, pharmaceutical compositions or kits comprising such, methods of using the gene editing systems to produce genetically modified cells, and the resultant cells thus produced. Also provided herein are uses of the gene editing systems disclosed herein, the pharmaceutical compositions and kits comprising such, and/or the genetically modified cells thus produced for treating primary hyperoxaluria (PH) in a subject.

In some aspects, the present disclosure features system for genetic editing of a hydroxyacid oxidase 1 (HAO1) gene, comprising (i) a Cas12i polypeptide or a first nucleic acid encoding the Cas12i polypeptide, and (ii) an RNA guide or a second nucleic acid encoding the RNA guide. The RNA guide comprises a spacer sequence specific to a target sequence within an HAO1 gene, the target sequence being adjacent to a protospacer adjacent motif (PAM) comprising the motif of 5′-TTN-3′, which is located 5′ to the target sequence.

In some embodiments, the Cas12i polypeptide can be a Cas12i2 polypeptide. In other embodiments, the Cas12i polypeptide can be a Cas12i4 polypeptide.

In some embodiments, the Cas12i polypeptide is a Cas12i2 polypeptide, which comprises an amino acid sequence at least 95% identical to SEQ ID NO: 922 and comprises one or more mutations relative to SEQ ID NO: 922. In some embodiments, the one or more mutations in the Cas12i2 polypeptide are at positions D581, G624, F626, P868, I926, V1030, E1035, and/or S1046 of SEQ ID NO: 922. In some examples, the one or more mutations are amino acid substitutions, which optionally is D581R, G624R, F626R, P868T, I926R, V1030G, E1035R, S1046G, or a combination thereof.

In one example, the Cas12i2 polypeptide comprises mutations at positions D581, D911, I926, and V1030 (e.g., amino acid substitutions of D581R, D911R, I926R, and V1030G). In another example, the Cas12i2 polypeptide comprises mutations at positions D581, I926, and V1030 (e.g., amino acid substitutions of D581R, I926R, and V1030G). In yet another example, the Cas12i2 polypeptide comprises mutations at positions D581, I926, V1030, and S1046 (e.g., amino acid substitutions of D581R, I926R, V1030G, and S1046G). In still another example, the Cas12i2 polypeptide comprises mutations at positions D581, G624, F626, I926, V1030, E1035, and S1046 (e.g., amino acid substitutions of D581R, G624R, F626R, I926R, V1030G, E1035R, and S1046G). In another example, the Cas12i2 polypeptide comprises mutations at positions D581, G624, F626, P868, I926, V1030, E1035, and S1046 (e.g., amino acid substitutions of D581R. G624R, F626R, P868T, I926R, V1030G, E1035R, and S1046G).

Exemplary Cas12i2 polypeptides for use in any of the gene editing systems disclosed herein may comprise the amino acid sequence of any one of SEQ ID NOs: 923-927. In one example, the exemplary Cas12i2 polypeptide for use in any of the gene editing systems disclosed herein comprises the amino acid sequence of SEQ ID NO: 924. In another example, the exemplary Cas12i2 polypeptide for use in any of the gene editing systems disclosed herein comprises the amino acid sequence of SEQ ID NO: 927.

In some embodiments, the gene editing system may comprise the first nucleic acid encoding the Cas12i polypeptide (e.g., the Cas12i2 polypeptide as disclosed herein). In some instances, the first nucleic acid is located in a first vector (e.g., a viral vector such as an adeno-associated viral vector or AAV vector). In some instances, the first nucleic acid is a messenger RNA (mRNA). In some instances, the nucleic acid encoding the Cas12i polypeptide (e.g., the Cas12i2 polypeptide as disclosed herein) is codon-optimized.

In some embodiments, the target sequence may be within exon 1 or exon 2 of the HAO1 gene. In some examples, the target sequence comprises 5′-CAAAGTCTATATATGACTAT-3′ (SEQ ID NO: 1025), 5′-GGAAGTACTGATTTAGCATG-3′ (SEQ ID NO: 1026), 5′-TAGATGGAAGCTGTATCCAA-3′ (SEQ ID NO: 1046), 5′-CGGAGCATCCTTGGATACAG-3′ (SEQ ID NO: 1047), or 5′-AGGACAGAGGGTCAGCATGC-3′ (SEQ ID NO: 1052). In specific examples, the target sequence can be the nucleotide sequence of SEQ ID NO: 1047.

In some embodiments, the spacer sequence may be 20-30-nucleotide in length. In some examples, the spacer sequence is 20-nucleotide in length. In some examples, the spacer sequence comprises 5′-CAAAGUCUAUAUAUGACUAU-3′ (SEQ ID NO: 1093); 5′-GGAAGUACUGAUUUAGCAUG-3′ (SEQ ID NO: 1094); 5′-UAGAUGGAAGCUGUAUCCAA-3′ (SEQ ID NO: 1095); 5′-CGGAGCAUCCUUGGAUACAG-3′ (SEQ ID NO: 1096); or 5′-AGGACAGAGGGUCAGCAUGC-3 (SEQ ID NO: 1097). In specific examples, the spacer sequence may comprise SEQ ID NO: 1096.

In some embodiments, the RNA guide comprises the spacer and a direct repeat sequence. In some examples, the direct repeat sequence is 23-36-nucleotide in length. In one example, the direct repeat sequence is at least 90% identical to any one of SEQ ID NOs: 1-10 or a fragment thereof that is at least 23-nucleotide in length. In some specific examples, the direct repeat sequence is any one of SEQ ID NOs: 1-10, or a fragment thereof that is at least 23-nucleotide in length. By way of non-limiting example, the direct repeat sequence is 5′-AGAAAUCCGUCUUUCAUUGACGG-3′ (SEQ ID NO: 10).

In specific examples, the RNA guide may comprise the nucleotide sequence of 5′-AGAAAUCCGUCUUUCAUUGACGGCAAAGUCUAUAUAUGACUAU-3′ (SEQ ID NO: 967), 5′-AGAAAUCCGUCUUUCAUUGACGGGGAAGUACUGAUUUAGCAUG-3′ (SEQ ID NO: 968), 5′-AGAAAUCCGUCUUUCAUUGACGGUAGAUGGAAGCUGUAUCCAA-3′ (SEQ ID NO: 988), 5′-AGAAAUCCGUCUUUCAUUGACGGCGGAGCAUCCUUGGAUACAG-3′ (SEQ ID NO: 989), or 5′-AGAAAUCCGUCUUUCAUUGACGGAGGACAGAGGGUCAGCAUGC-3′ (SEQ ID NO: 994). In specific examples, the RNA guide may comprise SEQ ID NO: 989.

In some embodiments, the system may comprise the second nucleic acid encoding the RNA guide. In some examples, the nucleic acid encoding the RNA guide may be located in a viral vector. In some examples, the viral vector comprises the both the first nucleic acid encoding the Cas12i2 polypeptide and the second nucleic acid encoding the RNA guide.

In some embodiments, any of the systems described herein may comprise the first nucleic acid encoding the Cas12i2 polypeptide, which is located in a first vector, and the second nucleic acid encoding the RNA guide, which is located on a second vector. In some examples, the first and/or second vector is a viral vector. In some specific examples, the first and second vectors are the same vector. In other examples, the first and second vectors are different vectors.

In some embodiments, any of the systems described herein may comprise one or more lipid nanoparticles (LNPs), which encompass the Cas12i2 polypeptide or the first nucleic acid encoding the Cas12i2 polypeptide, the RNA guide or the second nucleic acid encoding the RNA guide, or both.

In some embodiments, the system described herein may comprise a LNP, which encompass the Cas12i2 polypeptide or the first nucleic acid encoding the Cas12i2 polypeptide, and a viral vector comprising the second nucleic acid encoding the RNA guide. In some examples, the viral vector is an AAV vector. In other embodiments, the system described herein may comprise a LNP, which encompass the RNA guide or the second nucleic acid encoding the RNA guide, and a viral vector comprising the first nucleic acid encoding the Cas12i2 polypeptide. In some examples, the viral vector is an AAV vector.

In some aspects, the present disclosure also provides a pharmaceutical composition comprising any of the gene editing systems disclosed herein, or a kit comprising the components of the gene editing system.

In other aspects, the present disclosure also features a method for editing a hydroxyacid oxidase 1 (HAO1) gene in a cell, the method comprising contacting a host cell with any of the systems disclosed herein to genetically edit the HAO1 gene in the host cell. In some examples, the host cell is cultured in vitro. In other examples, the contacting step is performed by administering the system for editing the HAO1 gene to a subject comprising the host cell.

Also within the scope of the present disclosure is a cell comprising a disrupted a hydroxyacid oxidase 1 (HAO1) gene, which can be produced by contacting a host cell with the system disclosed herein genetically edit the HAO1 gene in the host cell.

Still in other aspects, the present disclosure provides a method for treating primary hyperoxaluria (PH) in a subject. The method may comprise administering to a subject in need thereof any of the systems for editing a hydroxyacid oxidase 1 (HAO1) gene or any of the modified cells disclosed herein. In some embodiments, the subject may be a human patient having the PH. In some examples, the PH is PH1, PH2, or PH3. In a specific example, the PH is PH1.

Also provided herein is an RNA guide, comprising (i) a spacer sequence as disclosed herein that is specific to a target sequence in a hydroxyacid oxidase 1 (HAO1) gene, wherein the target sequence is adjacent to a protospacer adjacent motif (PAM) comprising the motif of 5′-TTN-3′, which is located 5′ to the target sequence; and (ii) a direct repeat sequence.

In some embodiments, the spacer may be 20-30-nucleotide in length. In some examples, the spacer is 20-nucleotide in length.

In some embodiments, the direct repeat sequence may be 23-36-nucleotide in length. In some examples, the direct repeat sequence is 23-nucleotide in length.

In some embodiments, the target sequence may be within exon 1 or exon 2 of the HAO1 gene. In some examples, the target sequence comprises 5′-CAAAGTCTATATATGACTAT-3′ (SEQ ID NO: 1025), 5′-GGAAGTACTGATTTAGCATG-3′ (SEQ ID NO: 1026), 5′-TAGATGGAAGCTGTATCCAA-3′ (SEQ ID NO: 1046), 5′-CGGAGCATCCTTGGATACAG-3′ (SEQ ID NO: 1047), or 5′-AGGACAGAGGGTCAGCATGC-3′ (SEQ ID NO: 1052). In specific examples, the target sequence may comprise SEQ ID NO: 1047.

In some embodiments, the spacer sequence may be set forth as 5′-CAAAGUCUAUAUAUGACUAU-3′ (SEQ ID NO: 1093); 5′-GGAAGUACUGAUUUAGCAUG-3′ (SEQ ID NO:1094); 5′-UAGAUGGAAGCUGUAUCCAA-3′ (SEQ ID NO: 1095); 5′-CGGAGCAUCCUUGGAUACAG-3′ (SEQ ID NO: 1096); or 5′-AGGACAGAGGGUCAGCAUGC-3 (SEQ ID NO: 1097). In specific examples, the spacer sequence may comprise SEQ ID NO: 1096.

In some embodiments, the direct repeat sequence may be at least 90% identical to any one of SEQ ID NOs: 1-10 or a fragment thereof that is at least 23-nucleotide in length. In some examples, the direct repeat sequence is any one of SEQ ID NOs: 1-10, or a fragment thereof that is at least 23-nucleotide in length. By way of non-limiting example, the direct repeat sequence is 5′-AGAAAUCCGUCUUUCAUUGACGG-3′ (SEQ ID NO: 10).

In some embodiments, the RNA guide may comprise the nucleotide sequence of 5′-AGAAAUCCGUCUUUCAUUGACGGCAAAGUCUAUAUAUGACUAU-3′ (SEQ ID NO: 967), 5′-AGAAAUCCGUCUUUCAUUGACGGGGAAGUACUGAUUUAGCAUG-3′ (SEQ ID NO: 968), 5′-AGAAAUCCGUCUUUCAUUGACGGUAGAUGGAAGCUGUAUCCAA-3′ (SEQ ID NO: 988), 5′-AGAAAUCCGUCUUUCAUUGACGGCGGAGCAUCCUUGGAUACAG-3′ (SEQ ID NO: 989), or 5′-AGAAAUCCGUCUUUCAUUGACGGAGGACAGAGGGUCAGCAUGC-3′ (SEQ ID NO: 994). In specific examples, the RNA guide may comprise SEQ ID NO: 989.

Also provided herein are any of the gene editing systems disclosed herein, pharmaceutical compositions or kits comprising such, or genetically modified cells generated by the gene editing system for use in treating PH in a subject, as well as uses of the gene editing systems disclosed herein, pharmaceutical compositions or kits comprising such, or genetically modified cells generated by the gene editing system for manufacturing a medicament for treatment of PH in a subject.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein.

FIG. 1 is a graph showing the ability of RNPs prepared with a Cas12i2 polypeptide and a crRNA to edit the HAO1 gene in HEK293 cells. The darker grey bars represent target sequences with perfect homology to both rhesus macaque (Macaca mulatta) and crab-eating macaque (Macaca fascicularis) sequences.

FIG. 2 is a graph showing the ability of RNPs prepared with a Cas12i2 polypeptide and a crRNA to edit the HAO1 gene in HepG2 cells.

FIG. 3 is a graph showing the ability of RNPs prepared with a Cas12i2 polypeptide and a crRNA to edit the HAO1 gene in primary hepatocytes.

FIG. 4 is a graph showing knockdown of HAO1 mRNA in primary human hepatocytes with a Cas12i2 polypeptide and an HAO1-targeting crRNA.

FIG. 5A is a graph showing % indels induced by an HAO1-targeting crRNA and the variant Cas12i2 polypeptide of SEQ ID NO: 924 or SEQ ID NO: 927 in HepG2 cells. FIG. 5B shows the size (left) and start position (right) of indels induced in HepG2 cells by the variant Cas12i2 of SEQ ID NO: 924 and the HAO1-targeting RNA guide of E1T3 (SEQ ID NO: 968).

FIG. 6 is a graph showing % indels induced by chemically modified HAO1-targeting crRNAs of SEQ ID NO: 1091 and SEQ ID NO: 1092 and the variant Cas12i2 mRNA of SEQ ID NO: 1089 or SEQ ID NO: 1090.

FIG. 7A shows plots depicting tagmentation-based tag integration site sequencing (TTISS) reads for variant Cas12i2 of SEQ ID NO: 924 and HAO1-targeting RNA guides E2T5 (SEQ ID NO: 989), E1T2 (SEQ ID NO: 967), E1T3 (SEQ ID NO: 968), and E2T10 (SEQ ID NO: 994). The black wedge and centered number represent the fraction of on-target TTISS reads. Each gray wedge represents a unique off-target site identified by TTISS. The size of each gray wedge represents the fraction of TTISS reads mapping to a given off-target. FIG. 7B shows plots depicting two replicates of TTISS reads for variant Cas12i2 of SEQ ID NO: 927 and HAO1-targeting RNA guides E2T5 (SEQ ID NO: 989), E1T2 (SEQ ID NO: 967), and E1T3 (SEQ ID NO: 968). The black wedge and centered number represent the fraction of on-target TTISS reads. Each gray wedge represents a unique off-target site identified by TTISS. The size of each gray wedge represents the fraction of TTISS reads mapping to a given off-target.

FIG. 8 is a Western Blot showing knockdown of HAO1 protein following electroporation of primary human hepatocytes with variant Cas12i2 of SEQ ID NO: 924 and RNA guide E2T5 (SEQ ID NO: 989).

### DETAILED DESCRIPTION

The present disclosure relates to a system for genetic editing of a hydroxyacid oxidase 1 (HAO1) gene (a.k.a., glycolate oxidase gene), which comprises (i) a Cas12i polypeptide or a first nucleic acid encoding the Cas12i polypeptide, and (ii) an RNA guide or a second nucleic acid encoding the RNA guide, wherein the RNA guide comprises a spacer sequence specific to a target sequence within an HAO1 gene, the target sequence being adjacent to a protospacer adjacent motif (PAM) comprising the motif of 5′-TTN-3′, which is located 5′ to the target sequence. Also provided in the present disclosure are a pharmaceutical composition or a kit comprising such system as well as uses thereof. Further disclosed herein are a method for editing a HAO1 gene in a cell, a cell so produced that comprises a disrupted a HAO1 gene, a method of treating primary hyperoxaluria (PH) in a subject, and an RNA guide that comprises (i) a spacer that is specific to a target sequence in a HAO1 gene, wherein the target sequence is adjacent to a protospacer adjacent motif (PAM) comprising the motif of 5′-TTN-3′, which is located 5′ to the target sequence; and (ii) a direct repeat sequence as well as uses thereof.

The Cas12i polypeptide for use in the gene editing system disclosed herein may be a Cas12i2 polypeptide, e.g., a wild-type Cas12i polypeptide or a variant thereof as those disclosed herein. In some examples, the Cas12i2 polypeptide comprises an amino acid sequence at least 95% identical to SEQ ID NO: 922 and comprises one or more mutations relative to SEQ ID NO: 922. In other examples, the Cas12i polypeptide may be a Cas12i4 polypeptide, which is also disclosed herein.

### Definitions

The present disclosure will be described with respect to particular embodiments and with reference to certain Figures, but the disclosure is not limited thereto but only by the claims. Terms as set forth hereinafter are generally to be understood in their common sense unless indicated otherwise.

As used herein, the term “activity” refers to a biological activity. In some embodiments, activity includes enzymatic activity, e.g., catalytic ability of a Cas12i polypeptide. For example, activity can include nuclease activity.

As used herein the term “HAO1” refers to “glycolate oxidase 1,” which is also known as “hydroxyacid oxidase.” HAO1 is a peroxisome protein expressed primarily in the liver and pancreas, and its activities include oxidation of glycolate and 2-hydroxy fatty acids. SEQ ID NO: 928 as set forth herein provides an example of an HAO1 gene sequence.

As used herein, the term “Cas12i polypeptide” (also referred to herein as Cas12i) refers to a polypeptide that binds to a target sequence on a target nucleic acid specified by an RNA guide, wherein the polypeptide has at least some amino acid sequence homology to a wild-type Cas12i polypeptide. In some embodiments, the Cas12i polypeptide comprises at least 75%, at least 80%, at least 81%, at least 82%, at least 83%, at least 84%, at least 85%, at least 90%, at least 91%, at least 92%, at least 93%, at least 94%, at least 95%, at least 96%, at least 97%, at least 98%, at least 99% or 100% sequence identity with any one of SEQ ID NOs: 1-5 and 11-18 of U.S. Pat. No. 10,808,245, which is incorporated by reference for the subject matter and purpose referenced herein. In some embodiments, a Cas12i polypeptide comprises at least 75%, at least 80%, at least 81%, at least 82%, at least 83%, at least 84%, at least 85%, at least 90%, at least 91%, at least 92%, at least 93%, at least 94%, at least 95%, at least 96%, at least 97%, at least 98%, at least 99% or 100% sequence identity with any one of SEQ ID NOs: 8, 2, 11, and 9 of the present application. In some embodiments, a Cas12i polypeptide of the disclosure is a Cas12i2 polypeptide as described in WO/2021/202800, the relevant disclosures of which are incorporated by reference for the subject matter and purpose referenced herein. In some embodiments, the Cas12i polypeptide cleaves a target nucleic acid (e.g., as a nick or a double strand break).

As used herein, the term “adjacent to” refers to a nucleotide or amino acid sequence in close proximity to another nucleotide or amino acid sequence. In some embodiments, a nucleotide sequence is adjacent to another nucleotide sequence if no nucleotides separate the two sequences (i.e., immediately adjacent). In some embodiments, a nucleotide sequence is adjacent to another nucleotide sequence if a small number of nucleotides separate the two sequences (e.g., about 1, 2, 3, 4, 5, 6, 7, 8, 9, 10, 11, 12, 13, 14, 15, 16, 17, 18, 19, or 20 nucleotides). In some embodiments, a first sequence is adjacent to a second sequence if the two sequences are separated by about 5, 6, 7, 8, 9, 10, 11, 12, 13, 14, or 15 nucleotides. In some embodiments, a first sequence is adjacent to a second sequence if the two sequences are separated by up to 2 nucleotides, up to 5 nucleotides, up to 8 nucleotides, up to 10 nucleotides, up to 12 nucleotides, or up to 15 nucleotides. In some embodiments, a first sequence is adjacent to a second sequence if the two sequences are separated by 2-5 nucleotides, 4-6 nucleotides, 4-8 nucleotides, 4-10 nucleotides, 6-8 nucleotides, 6-10 nucleotides, 6-12 nucleotides, 8-10 nucleotides, 8-12 nucleotides, 10-12 nucleotides, 10-15 nucleotides, or 12-15 nucleotides.

As used herein, the term “complex” refers to a grouping of two or more molecules. In some embodiments, the complex comprises a polypeptide and a nucleic acid molecule interacting with (e.g., binding to, coming into contact with, adhering to) one another. For example, the term “complex” can refer to a grouping of an RNA guide and a polypeptide (e.g., a Cas12i polypeptide). Alternatively, the term “complex” can refer to a grouping of an RNA guide, a polypeptide, and the complementary region of a target sequence. In another example, the term “complex” can refer to a grouping of an HAO1-targeting RNA guide and a Cas12i polypeptide.

As used herein, the term “protospacer adjacent motif” or “PAM” refers to a DNA sequence adjacent to a target sequence (e.g., an HAO1 target sequence) to which a complex comprising an RNA guide (e.g., an HAO1-targeting RNA guide) and a Cas12i polypeptide binds. In a double-stranded DNA molecule, the strand containing the PAM motif is called the “PAM-strand” and the complementary strand is called the “non-PAM strand.” The RNA guide binds to a site in the non-PAM strand that is complementary to a target sequence disclosed herein.

In some embodiments, the PAM strand is a coding (e.g., sense) strand. In other embodiments, the PAM strand is a non-coding (e.g., antisense strand). Since an RNA guide binds the non-PAM strand via base-pairing, the non-PAM strand is also known as the target strand, while the PAM strand is also known as the non-target strand.

As used herein, the term “target sequence” refers to a DNA fragment adjacent to a PAM motif (on the PAM strand). The complementary region of the target sequence is on the non-PAM strand. A target sequence may be immediately adjacent to the PAM motif. Alternatively, the target sequence and the PAM may be separately by a small sequence segment (e.g., up to 5 nucleotides, for example, up to 4, 3, 2, or 1 nucleotide). A target sequence may be located at the 3′ end of the PAM motif or at the 5′ end of the PAM motif, depending upon the CRISPR nuclease that recognizes the PAM motif, which is known in the art. For example, a target sequence is located at the 3′ end of a PAM motif for a Cas12i polypeptide (e.g., a Cas12i2 polypeptide such as those disclosed herein). In some embodiments, the target sequence is a sequence within an HAO1 gene sequence, including, but not limited, to the sequence set forth in SEQ ID NO: 928.

As used herein, the term “adjacent to” refers to a nucleotide or amino acid sequence in close proximity to another nucleotide or amino acid sequence. In some embodiments, a nucleotide sequence is adjacent to another nucleotide sequence if no nucleotides separate the two sequences (i.e., immediately adjacent). In some embodiments, a nucleotide sequence is adjacent to another nucleotide sequence if a small number of nucleotides separate the two sequences (e.g., about 1, 2, 3, 4, 5, 6, 7, 8, 9, 10, 11, 12, 13, 14, 15, 16, 17, 18, 19, or 20 nucleotides). In some embodiments, a first sequence is adjacent to a second sequence if the two sequences are separated by about 5, 6, 7, 8, 9, 10, 11, 12, 13, 14, or 15 nucleotides. In some embodiments, a first sequence is adjacent to a second sequence if the two sequences are separated by up to 2 nucleotides, up to 5 nucleotides, up to 8 nucleotides, up to 10 nucleotides, up to 12 nucleotides, or up to 15 nucleotides. In some embodiments, a first sequence is adjacent to a second sequence if the two sequences are separated by 2-5 nucleotides, 4-6 nucleotides, 4-8 nucleotides, 4-10 nucleotides, 6-8 nucleotides, 6-10 nucleotides, 6-12 nucleotides, 8-10 nucleotides, 8-12 nucleotides, 10-12 nucleotides, 10-15 nucleotides, or 12-15 nucleotides.

As used herein, the term “spacer” or “spacer sequence” is a portion in an RNA guide that is the RNA equivalent of the target sequence (a DNA sequence). The spacer contains a sequence capable of binding to the non-PAM strand via base-pairing at the site complementary to the target sequence (in the PAM strand). Such a spacer is also known as specific to the target sequence. In some instances, the spacer may be at least 75% identical to the target sequence (e.g., at least 80%, at least 85%, at least 90%, at least 95%, at least 98%, or at least 99%), except for the RNA-DNA sequence difference. In some instances, the spacer may be 100% identical to the target sequence except for the RNA-DNA sequence difference.

As used herein, the term “RNA guide” or “RNA guide sequence” refers to any RNA molecule or a modified RNA molecule that facilitates the targeting of a polypeptide (e.g., a Cas12i polypeptide) described herein to a target sequence (e.g., a sequence of an HAO1 gene). For example, an RNA guide can be a molecule that is designed to be complementary to a specific nucleic acid sequence (a target sequence such as a target sequence with an HAO1 gene). An RNA guide may comprise a spacer sequence and a direct repeat (DR) sequence. In some instances, the RNA guide can be a modified RNA molecule comprising one or more deoxyribonucleotides, for example, in a DNA-binding sequence contained in the RNA guide, which binds a sequence complementary to the target sequence. In some examples, the DNA-binding sequence may contain a DNA sequence or a DNA/RNA hybrid sequence. The terms CRISPR RNA (crRNA), pre-crRNA and mature crRNA are also used herein to refer to an RNA guide.

As used herein, the term “complementary” refers to a first polynucleotide (e.g., a spacer sequence of an RNA guide) that has a certain level of complementarity to a second polynucleotide (e.g., the complementary sequence of a target sequence) such that the first and second polynucleotides can form a double-stranded complex via base-pairing to permit an effector polypeptide that is complexed with the first polynucleotide to act on (e.g., cleave) the second polynucleotide. In some embodiments, the first polynucleotide may be substantially complementary to the second polynucleotide, i.e., having at least about 80%, 81%, 82%, 83%, 84%, 85%, 86%, 87%, 88%, 89%, 90%, 91%, 92%, 93%, 94%, 95%, 96%, 97%, 98%, or 99% complementarity to the second polynucleotide. In some embodiments, the first polynucleotide is completely complementary to the second polynucleotide, i.e., having 100% complementarity to the second polynucleotide.

The “percent identity” (a.k.a., sequence identity) of two nucleic acids or of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST nucleotide searches can be performed with the NBLAST program, score=100, wordlength-12 to obtain nucleotide sequences homologous to the nucleic acid molecules of the invention. BLAST protein searches can be performed with the XBLAST program, score=50, word length=3 to obtain amino acid sequences homologous to the protein molecules of the invention. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

As used herein, the term “edit” refers to one or more modifications introduced into a target nucleic acid, e.g., within the HAO1 gene. The edit can be one or more substitutions, one or more insertions, one or more deletions, or a combination thereof. As used herein, the term “substitution” refers to a replacement of a nucleotide or nucleotides with a different nucleotide or nucleotides, relative to a reference sequence. As used herein, the term “insertion” refers to a gain of a nucleotide or nucleotides in a nucleic acid sequence, relative to a reference sequence. As used herein, the term “deletion” refers to a loss of a nucleotide or nucleotides in a nucleic acid sequence, relative to a reference sequence.

No particular process is implied in how to make a sequence comprising a deletion. For instance, a sequence comprising a deletion can be synthesized directly from individual nucleotides. In other embodiments, a deletion is made by providing and then altering a reference sequence. The nucleic acid sequence can be in a genome of an organism. The nucleic acid sequence can be in a cell. The nucleic acid sequence can be a DNA sequence. The deletion can be a frameshift mutation or a non-frameshift mutation. A deletion described herein refers to a deletion of up to several kilobases.

As used herein, the terms “upstream” and “downstream” refer to relative positions within a single nucleic acid (e.g., DNA) sequence in a nucleic acid molecule. “Upstream” and “downstream” relate to the 5′ to 3′ direction, respectively, in which RNA transcription occurs. A first sequence is upstream of a second sequence when the 3′ end of the first sequence occurs before the 5′ end of the second sequence. A first sequence is downstream of a second sequence when the 5′ end of the first sequence occurs after the 3′ end of the second sequence. In some embodiments, the 5′-NTTN-3′ or 5′-TTN-3′ sequence is upstream of an indel described herein, and a Cas12i-induced indel is downstream of the 5′-NTTN-3′ or 5′-TTN-3′ sequence.

### I. Gene Editing Systems

In some aspects, the present disclosure provides gene editing systems comprising an RNA guide targeting an HAO1 gene. Such a gene editing system can be used to edit the HAO1 target gene, e.g., to disrupt the HAO1 gene.

Hydroxyacid oxidase 1 (HAO1, also known as glycolate oxidase [GOX or GO]), converts glycolate into glyoxylate. It has been proposed that inhibition of HAO1 in individuals with PH1 would block formation of glyoxylate, and excess glycolate would be excreted through the urine. The idea of treating PH1 by inhibition of HAO1 is further supported that some individuals with abnormal splice variants of HAO1 are asymptomatic for glycolic aciduria, whereby there was increased urinary glycolic acid excretion without apparent kidney pathology. Thus, inhibition of HAO1 expression would block production of glyoxylate, and in turn block production of its metabolite, oxalate. Accordingly, the gene editing systems disclosed here, targeting the HAO1 gene, could be used to treat primary hyperoxaluria (PH) in a subject in need of the treatment.

In some embodiments, the RNA guide is comprised of a direct repeat component and a spacer component. In some embodiments, the RNA guide binds a Cas12i polypeptide. In some embodiments, the spacer component is specific to an HAO1 target sequence, wherein the HAO1 target sequence is adjacent to a 5′-NTTN-3′ or 5′-TTN-3′ PAM sequence as described herein. In the case of a double-stranded target, the RNA guide binds to a first strand of the target (i.e., the non-PAM strand) and a PAM sequence as described herein is present in the second, complementary strand (i.e., the PAM strand).

In some embodiments, the present disclosure provides compositions comprising a complex, wherein the complex comprises an RNA guide targeting HAO1. In some embodiments, the present disclosure comprises a complex comprising an RNA guide and a Cas12i polypeptide. In some embodiments, the RNA guide and the Cas12i polypeptide bind to each other in a molar ratio of about 1:1. In some embodiments, a complex comprising an RNA guide and a Cas12i polypeptide binds to the complementary region of a target sequence within an HAO1 gene. In some embodiments, a complex comprising an RNA guide targeting HAO1 and a Cas12i polypeptide binds to the complementary region of a target sequence within an HAO1 gene at a molar ratio of about 1:1. In some embodiments, the complex comprises enzymatic activity, such as nuclease activity, that can cleave the HAO1 target sequence and/or the complementary sequence. The RNA guide, the Cas12i polypeptide, and the complementary region of the HAO1 target sequence, either alone or together, do not naturally occur. In some embodiments, the RNA guide in the complex comprises a direct repeat and/or a spacer sequence described herein. In some embodiments, the sequence of the RNA guide has at least 90% identity (e.g., at least 90%, 91%, 92%, 93%, 94%, 95%, 96%, 97%, 98%, or 99% identity) to a sequence of any one of SEQ ID NOs: 967-1023. In some embodiments, the RNA guide has a sequence of any one of SEQ ID NOs: 967-1023.

In some embodiments, the present disclosure described herein comprises compositions comprising an RNA guide as described herein and/or an RNA encoding a Cas12i polypeptide as described herein. In some embodiments, the RNA guide and the RNA encoding a Cas12i polypeptide are comprised together within the same composition. In some embodiments, the RNA guide and the RNA encoding a Cas12i polypeptide are comprised within separate compositions. In some embodiments, the RNA guide comprises a direct repeat and/or a spacer sequence described herein. In some embodiments, the sequence of the RNA guide has at least 90% identity (e.g., at least 90%, 91%, 92%, 93%, 94%, 95%, 96%, 97%, 98%, or 99% identity) to a sequence of any one of SEQ ID NOs: 967-1023. In some embodiments, the RNA guide has a sequence of any one of SEQ ID NOs: 967-1023.

Use of the gene editing systems disclosed herein has advantages over those of other known nuclease systems. Cas12i polypeptides are smaller than other nucleases. For example, Cas12i2 is 1,054 amino acids in length, whereas S. pyogenes Cas9 (SpCas9) is 1.368 amino acids in length, S. thermophilus Cas9 (StCas9) is 1,128 amino acids in length, FnCpf1 is 1,300 amino acids in length, AsCpf1 is 1,307 amino acids in length, and LbCpf1 is 1,246 amino acids in length. Cas12i RNA guides, which do not require a trans-activating CRISPR RNA (tracrRNA), are also smaller than Cas9 RNA guides. The smaller Cas12i polypeptide and RNA guide sizes are beneficial for delivery. Compositions comprising a Cas12i polypeptide also demonstrate decreased off-target activity compared to compositions comprising an SpCas9 polypeptide. See PCT/US2021/025257, which is incorporated by reference in its entirety. Furthermore, indels induced by compositions comprising a Cas12i polypeptide differ from indels induced by compositions comprising an SpCas9 polypeptide. For example, SpCas9 polypeptides primarily induce insertions and deletions of 1 nucleotide in length. However, Cas12i polypeptides induce larger deletions, which can be beneficial in disrupting a larger portion of a gene such as HAO1.

Also provided herein is a system for genetic editing of a hydroxyacid oxidase 1 (HAO1) gene, which comprises (i) a Cas12i polypeptide (e.g., a Cas12i2 polypeptide) or a first nucleic acid encoding the Cas12i polypeptide (e.g., a Cas12i2 polypeptide comprises an amino acid sequence at least 95% identical to SEQ ID NO: 922, which may and comprises one or more mutations relative to SEQ ID NO: 922); and (ii) an RNA guide or a second nucleic acid encoding the RNA guide, wherein the RNA guide comprises a spacer sequence specific to a target sequence within an HAO1 gene (e.g., within exon 1 or exon 2 of the HAO1 gene), the target sequence being adjacent to a protospacer adjacent motif (PAM) comprising the motif of 5′-TTN-3′ (5′-NTTN-3′), which is located 5′ to the target sequence.

### A. RNA Guides

In some embodiments, the gene editing system described herein comprises an RNA guide targeting a HAO1 gene, for example, targeting exon 1 or exon 2 of the HAO1 gene. In some embodiments, the gene editing system described herein may comprise two or more (e.g., 2, 3, 4, 5, 6, 7, 8, 9, or more) RNA guides targeting HAO1.

The RNA guide may direct the Cas12i polypeptide contained in the gene editing system as described herein to an HAO1 target sequence. Two or more RNA guides may direct two or more separate Cas12i polypeptides (e.g., Cas12i polypeptides having the same or different sequence) as described herein to two or more (e.g., 2, 3, 4, 5, 6, 7, 8, 9, or more) HAO1 target sequences.

Those skilled in the art reading the below examples of particular kinds of RNA guides will understand that, in some embodiments, an RNA guide is HAO1 target-specific. That is, in some embodiments, an RNA guide binds specifically to one or more HAO1 target sequences (e.g., within a cell) and not to non-targeted sequences (e.g., non-specific DNA or random sequences within the same cell).

In some embodiments, the RNA guide comprises a spacer sequence followed by a direct repeat sequence, referring to the sequences in the 5′ to 3′ direction. In some embodiments, the RNA guide comprises a first direct repeat sequence followed by a spacer sequence and a second direct repeat sequence, referring to the sequences in the 5′ to 3′ direction. In some embodiments, the first and second direct repeats of such an RNA guide are identical. In some embodiments, the first and second direct repeats of such an RNA guide are different.

In some embodiments, the spacer sequence and the direct repeat sequence(s) of the RNA guide are present within the same RNA molecule. In some embodiments, the spacer and direct repeat sequences are linked directly to one another. In some embodiments, a short linker is present between the spacer and direct repeat sequences, e.g., an RNA linker of 1, 2, or 3 nucleotides in length. In some embodiments, the spacer sequence and the direct repeat sequence(s) of the RNA guide are present in separate molecules, which are joined to one another by base pairing interactions.

Additional information regarding exemplary direct repeat and spacer components of RNA guides is provided as follows.

### (i). Direct Repeat

In some embodiments, the RNA guide comprises a direct repeat sequence. In some embodiments, the direct repeat sequence of the RNA guide has a length of between 12-100, 13-75, 14-50, or 15-40 nucleotides (e.g., 15, 16, 17, 18, 19, 20, 21, 22, 23, 24, 25, 26, 27, 28, 29, 30, 31, 32, 33, 34, 35, 36, 37, 38, 39, or 40 nucleotides).

In some embodiments, the direct repeat sequence is a sequence of Table 1 or a portion of a sequence of Table 1. The direct repeat sequence can comprise nucleotide 1 through nucleotide 36 of any one of SEQ ID NOs: 1, 2, 3, 4, 5, 6, 7, or 8. The direct repeat sequence can comprise nucleotide 2 through nucleotide 36 of any one of SEQ ID NOs: 1, 2, 3, 4, 5, 6, 7, or 8. The direct repeat sequence can comprise nucleotide 3 through nucleotide 36 of any one of SEQ ID NOs: 1, 2, 3, 4, 5, 6, 7, or 8. The direct repeat sequence can comprise nucleotide 4 through nucleotide 36 of any one of SEQ ID NOs: 1, 2, 3, 4, 5, 6, 7, or 8. The direct repeat sequence can comprise nucleotide 5 through nucleotide 36 of any one of SEQ ID NOs: 1, 2, 3, 4, 5, 6, 7, or 8. The direct repeat sequence can comprise nucleotide 6 through nucleotide 36 of any one of SEQ ID NOs: 1, 2, 3, 4, 5, 6, 7, or 8. The direct repeat sequence can comprise nucleotide 7 through nucleotide 36 of any one of SEQ ID NOs: 1, 2, 3, 4, 5, 6, 7, or 8. The direct repeat sequence can comprise nucleotide 8 through nucleotide 36 of any one of SEQ ID NOs: 1, 2, 3, 4, 5, 6, 7, or 8. The direct repeat sequence can comprise nucleotide 9 through nucleotide 36 of any one of SEQ ID NOs: 1, 2, 3, 4, 5, 6, 7, or 8. The direct repeat sequence can comprise nucleotide 10 through nucleotide 36 of any one of SEQ ID NOs: 1, 2, 3, 4, 5, 6, 7, or 8. The direct repeat sequence can comprise nucleotide 11 through nucleotide 36 of any one of SEQ ID NOs: 1, 2, 3, 4, 5, 6, 7, or 8. The direct repeat sequence can comprise nucleotide 12 through nucleotide 36 of any one of SEQ ID NOs: 1, 2, 3, 4, 5, 6, 7, or 8. The direct repeat sequence can comprise nucleotide 13 through nucleotide 36 of any one of SEQ ID NOs: 1, 2, 3, 4, 5, 6, 7, or 8. The direct repeat sequence can comprise nucleotide 14 through nucleotide 36 of any one of SEQ ID NOs: 1, 2, 3, 4, 5, 6, 7, or 8.

The direct repeat sequence can comprise nucleotide 1 through nucleotide 34 of SEQ ID NO: 9. The direct repeat sequence can comprise nucleotide 2 through nucleotide 34 of SEQ ID NO: 9. The direct repeat sequence can comprise nucleotide 3 through nucleotide 34 of SEQ ID NO: 9. The direct repeat sequence can comprise nucleotide 4 through nucleotide 34 of SEQ ID NO: 9. The direct repeat sequence can comprise nucleotide 5 through nucleotide 34 of SEQ ID NO: 9. The direct repeat sequence can comprise nucleotide 6 through nucleotide 34 of SEQ ID NO: 9. The direct repeat sequence can comprise nucleotide 7 through nucleotide 34 of SEQ ID NO: 9. The direct repeat sequence can comprise nucleotide 8 through nucleotide 34 of SEQ ID NO: 9. The direct repeat sequence can comprise nucleotide 9 through nucleotide 34 of SEQ ID NO: 9. The direct repeat sequence can comprise nucleotide 10 through nucleotide 34 of SEQ ID NO: 9. The direct repeat sequence can comprise nucleotide 11 through nucleotide 34 of SEQ ID NO: 9. The direct repeat sequence can comprise nucleotide 12 through nucleotide 34 of SEQ ID NO: 9. In some embodiments, the direct repeat sequence is set forth in SEQ ID NO: 10. In some embodiments, the direct repeat sequence comprises a portion of the sequence set forth in SEQ ID NO: 10.

In some embodiments, the direct repeat sequence has at least 90% identity (e.g., at least 90%, 91%, 92%, 93%, 94%, 95%, 96%, 97%, 98% or 99% identity) to a sequence of Table 1 or a portion of a sequence of Table 1. The direct repeat sequence can have at least 90% identity to a sequence comprising nucleotide 1 through nucleotide 36 of any one of SEQ ID NOs: 1, 2, 3, 4, 5, 6, 7, or 8. The direct repeat sequence can have at least 90% identity to a sequence comprising 2 through nucleotide 36 of any one of SEQ ID NOs: 1, 2, 3, 4, 5, 6, 7, or 8. The direct repeat sequence can have at least 90% identity to a sequence comprising 3 through nucleotide 36 of any one of SEQ ID NOs: 1, 2, 3, 4, 5, 6, 7, or 8. The direct repeat sequence can have at least 90% identity to a sequence comprising 4 through nucleotide 36 of any one of SEQ ID NOs: 1, 2, 3, 4, 5, 6, 7, or 8. The direct repeat sequence can have at least 90% identity to a sequence comprising 5 through nucleotide 36 of any one of SEQ ID NOs: 1, 2, 3, 4, 5, 6, 7, or 8. The direct repeat sequence can have at least 90% identity to a sequence comprising 6 through nucleotide 36 of any one of SEQ ID NOs: 1, 2, 3, 4, 5, 6, 7, or 8. The direct repeat sequence can have at least 90% identity to a sequence comprising 7 through nucleotide 36 of any one of SEQ ID NOs: 1, 2, 3, 4, 5, 6, 7, or 8. The direct repeat sequence can have at least 90% identity to a sequence comprising 8 through nucleotide 36 of any one of SEQ ID NOs: 1, 2, 3, 4, 5, 6, 7, or 8. The direct repeat sequence can have at least 90% identity to a sequence comprising 9 through nucleotide 36 of any one of SEQ ID NOs: 1, 2, 3, 4, 5, 6, 7, or 8. The direct repeat sequence can have at least 90% identity to a sequence comprising 10 through nucleotide 36 of any one of SEQ ID NOs: 1, 2, 3, 4, 5, 6, 7, or 8. The direct repeat sequence can have at least 90% identity to a sequence comprising 11 through nucleotide 36 of any one of SEQ ID NOs: 1, 2, 3, 4, 5, 6, 7, or 8. The direct repeat sequence can have at least 90% identity to a sequence comprising 12 through nucleotide 36 of any one of SEQ ID NOs: 1, 2, 3, 4, 5, 6, 7, or 8. The direct repeat sequence can have at least 90% identity to a sequence comprising 13 through nucleotide 36 of any one of SEQ ID NOs: 1, 2, 3, 4, 5, 6, 7, or 8. The direct repeat sequence can have at least 90% identity to a sequence comprising 14 through nucleotide 36 of any one of SEQ ID NOs: 1, 2, 3, 4, 5, 6, 7, or 8. The direct repeat sequence can have at least 90% identity to a sequence comprising 1 through nucleotide 34 of SEQ ID NO: 9. The direct repeat sequence can have at least 90% identity to a sequence comprising 2 through nucleotide 34 of SEQ ID NO: 9. The direct repeat sequence can have at least 90% identity to a sequence comprising 3 through nucleotide 34 of SEQ ID NO: 9.

The direct repeat sequence can have at least 90% identity to a sequence comprising 4 through nucleotide 34 of SEQ ID NO: 9. The direct repeat sequence can have at least 90% identity to a sequence comprising 5 through nucleotide 34 of SEQ ID NO: 9. The direct repeat sequence can have at least 90% identity to a sequence comprising 6 through nucleotide 34 of SEQ ID NO: 9. The direct repeat sequence can have at least 90% identity to a sequence comprising 7 through nucleotide 34 of SEQ ID NO: 9. The direct repeat sequence can have at least 90% identity to a sequence comprising 8 through nucleotide 34 of SEQ ID NO: 9. The direct repeat sequence can have at least 90% identity to a sequence comprising 9 through nucleotide 34 of SEQ ID NO: 9. The direct repeat sequence can have at least 90% identity to a sequence comprising 10 through nucleotide 34 of SEQ ID NO: 9. The direct repeat sequence can have at least 90% identity to a sequence comprising 11 through nucleotide 34 of SEQ ID NO: 9.

The direct repeat sequence can have at least 90% identity to a sequence comprising 12 through nucleotide 34 of SEQ ID NO: 9. In some embodiments, the direct repeat sequence has at least 90% identity (e.g., at least 90%, 91%, 92%, 93%, 94%, 95%, 96%, 97%, 98% or 99% identity) to SEQ ID NO: 10. In some embodiments, the direct repeat sequence has at least 90% identity to a portion of the sequence set forth in SEQ ID NO: 10.

In some embodiments, compositions comprising a Cas12i2 polypeptide and an RNA guide comprising the direct repeat of SEQ ID NO: 10 and a spacer length of 20 nucleotides are capable of introducing indels into an HAO1 target sequence. See, e.g., Example 1, where indels were measured at forty-four HAO1 target sequences following delivery of an RNA guide and a Cas12i2 polypeptide of SEQ ID NO: 924 to HEK293T cells by RNP; Example 2, where indels were measured at eleven HAO1 target sequences following delivery of an RNA guide and a Cas12i2 polypeptide of SEQ ID NO: 924 to HepG2 cells by RNP; and Example 3, where indels were measured at five HAO1 target sequences following delivery of an RNA guide and a Cas12i2 polypeptide of SEQ ID NO: 924 to primary hepatocytes by RNP.

In some embodiments, the direct repeat sequence is at least 90% identical to the reverse complement of any one of SEQ ID NOs: 1-10 (see, Table 1). In some embodiments, the direct repeat sequence is the reverse complement of any one of SEQ ID NOs: 1-10.

TABLE 1
Cas12i2 Direct Repeat Sequences

| Sequence | |
|---|---|
| identifier | Direct Repeat Sequence |
| SEQ ID NO: 1 | GUUGCAAAACCCAAGAAAUCCGUCUUUCAUUGACGG |
| SEQ ID NO: 2 | AAUAGCGGCCCUAAGAAAUCCGUCUUUCAUUGACGG |
| SEQ ID NO: 3 | AUUGGAACUGGCGAGAAAUCCGUCUUUCAUUGACGG |
| SEQ ID NO: 4 | CCAGCAACACCUAAGAAAUCCGUCUUUCAUUGACGG |
| SEQ ID NO: 5 | CGGCGCUCGAAUAGGAAAUCCGUCUUUCAUUGACGG |
| SEQ ID NO: 6 | GUGGCAACACCUAAGAAAUCCGUCUUUCAUUGACGG |
| SEQ ID NO: 7 | GUUGCAACACCUAAGAAAUCCGUCUUUCAUUGACGG |
| SEQ ID NO: 8 | GUUGCAAUGCCUAAGAAAUCCGUCUUUCAUUGACGG |
| SEQ ID NO: 9 | GCAACACCUAAGAAAUCCGUCUUUCAUUGACGGG |
| SEQ ID NO: 10 | AGAAAUCCGUCUUUCAUUGACGG |

In some embodiments, the direct repeat sequence is a sequence of Table 2 or a portion of a sequence of Table 2. The direct repeat sequence can comprise nucleotide 1 through nucleotide 36 of any one of SEQ ID NOs: 936, 937, 938, 939, 940, 941, 942, 943, 944, 945, 946, 947, 948, 949, 950, 951, 952, or 953. The direct repeat sequence can comprise nucleotide 2 through nucleotide 36 of any one of SEQ ID NOs: 936, 937, 938, 939, 940, 941, 942, 943, 944, 945, 946, 947, 948, 949, 950, 951, 952, or 953. The direct repeat sequence can comprise nucleotide 3 through nucleotide 36 of any one of SEQ ID NOs: 936, 937, 938, 939, 940, 941, 942, 943, 944, 945, 946, 947, 948, 949, 950, 951, 952, or 953. The direct repeat sequence can comprise nucleotide 4 through nucleotide 36 of any one of SEQ ID NOs: 936, 937, 938, 939, 940, 941, 942, 943, 944, 945, 946, 947, 948, 949, 950, 951, 952, or 953. The direct repeat sequence can comprise nucleotide 5 through nucleotide 36 of any one of SEQ ID NOs: 936, 937, 938, 939, 940, 941, 942, 943, 944, 945, 946, 947, 948, 949, 950, 951, 952, or 953. The direct repeat sequence can comprise nucleotide 6 through nucleotide 36 of any one of SEQ ID NOs: 936, 937, 938, 939, 940, 941, 942, 943, 944, 945, 946, 947, 948, 949, 950, 951, 952, or 953. The direct repeat sequence can comprise nucleotide 7 through nucleotide 36 of any one of SEQ ID NOS: 936, 937, 938, 939, 940, 941, 942, 943, 944, 945, 946, 947, 948, 949, 950, 951, 952, or 953. The direct repeat sequence can comprise nucleotide 8 through nucleotide 36 of any one of SEQ ID NOs: 936, 937, 938, 939, 940, 941, 942, 943, 944, 945, 946, 947, 948, 949, 950, 951, 952, or 953. The direct repeat sequence can comprise nucleotide 9 through nucleotide 36 of any one of SEQ ID NOs: 936, 937, 938, 939, 940, 941, 942, 943, 944, 945, 946, 947, 948, 949, 950, 951, 952, or 953. The direct repeat sequence can comprise nucleotide 10 through nucleotide 36 of any one of SEQ ID NOs: 936, 937, 938, 939, 940, 941, 942, 943, 944, 945, 946, 947, 948, 949, 950, 951, 952, or 953. The direct repeat sequence can comprise nucleotide 11 through nucleotide 36 of any one of SEQ ID NOs: 936, 937, 938, 939, 940, 941, 942, 943, 944, 945, 946, 947, 948, 949, 950, 951, 952, or 953. The direct repeat sequence can comprise nucleotide 12 through nucleotide 36 of any one of SEQ ID NOs: 936, 937, 938, 939, 940, 941, 942, 943, 944, 945, 946, 947, 948, 949, 950, 951, 952, or 953. The direct repeat sequence can comprise nucleotide 13 through nucleotide 36 of any one of SEQ ID NOs: 936, 937, 938, 939, 940, 941, 942, 943, 944, 945, 946, 947, 948, 949, 950, 951, 952, or 953. The direct repeat sequence can comprise nucleotide 14 through nucleotide 36 of any one of SEQ ID NOs: 936, 937, 938, 939, 940, 941, 942, 943, 944, 945, 946, 947, 948, 949, 950, 951, 952, or 953.

In some embodiments, the direct repeat sequence has at least 95% identity (e.g., at least 95%, 96%, 97%, 98% or 99% identity) to a sequence of Table 2 or a portion of a sequence of Table 2. The direct repeat sequence can have at least 95% identity to a sequence comprising nucleotide 1 through nucleotide 36 of any one of SEQ ID NOs: 936, 937, 938, 939, 940, 941, 942, 943, 944, 945, 946, 947, 948, 949, 950, 951, 952, or 953. The direct repeat sequence can have at least 95% identity to a sequence comprising 2 through nucleotide 36 of any one of SEQ ID NOs: 936, 937, 938, 939, 940, 941, 942, 943, 944, 945, 946, 947, 948, 949, 950, 951, 952, or 953. The direct repeat sequence can have at least 95% identity to a sequence comprising 3 through nucleotide 36 of any one of SEQ ID NOs: 936, 937, 938, 939, 940, 941, 942, 943, 944, 945, 946, 947, 948, 949, 950, 951, 952, or 953. The direct repeat sequence can have at least 95% identity to a sequence comprising 4 through nucleotide 36 of any one of SEQ ID NOs: 936, 937, 938, 939, 940, 941, 942, 943, 944, 945, 946, 947, 948, 949, 950, 951, 952, or 953. The direct repeat sequence can have at least 95% identity to a sequence comprising 5 through nucleotide 36 of any one of SEQ ID NOs: 936, 937, 938, 939, 940, 941, 942, 943, 944, 945, 946, 947, 948, 949, 950, 951, 952, or 953. The direct repeat sequence can have at least 95% identity to a sequence comprising 6 through nucleotide 36 of any one of SEQ ID NOs: 936, 937, 938, 939, 940, 941, 942, 943, 944, 945, 946, 947, 948, 949, 950, 951, 952, or 953. The direct repeat sequence can have at least 95% identity to a sequence comprising 7 through nucleotide 36 of any one of SEQ ID NOs: 936, 937, 938, 939, 940, 941, 942, 943, 944, 945, 946, 947, 948, 949, 950, 951, 952, or 953. The direct repeat sequence can have at least 95% identity to a sequence comprising 8 through nucleotide 36 of any one of SEQ ID NOs: 936, 937, 938, 939, 940, 941, 942, 943, 944, 945, 946, 947, 948, 949, 950, 951, 952, or 953. The direct repeat sequence can have at least 95% identity to a sequence comprising 9 through nucleotide 36 of any one of SEQ ID NOs: 936, 937, 938, 939, 940, 941, 942, 943, 944, 945, 946, 947, 948, 949, 950, 951, 952, or 953. The direct repeat sequence can have at least 95% identity to a sequence comprising 10 through nucleotide 36 of any one of SEQ ID NOs: 936, 937, 938, 939, 940, 941, 942, 943, 944, 945, 946, 947, 948, 949, 950, 951, 952, or 953. The direct repeat sequence can have at least 95% identity to a sequence comprising 11 through nucleotide 36 of any one of SEQ ID NOs: 936, 937, 938, 939, 940, 941, 942, 943, 944, 945, 946, 947, 948, 949, 950, 951, 952, or 953. The direct repeat sequence can have at least 95% identity to a sequence comprising 12 through nucleotide 36 of any one of SEQ ID NOs: 936, 937, 938, 939, 940, 941, 942, 943, 944, 945, 946, 947, 948, 949, 950, 951, 952, or 953. The direct repeat sequence can have at least 95% identity to a sequence comprising 13 through nucleotide 36 of any one of SEQ ID NOs: 936, 937, 938, 939, 940, 941, 942, 943, 944, 945, 946, 947, 948, 949, 950, 951, 952, or 953.

In some embodiments, the direct repeat sequence has at least 90% identity (e.g., at least 90%. 91%, 92%, 93%, 94%, 95%, 96%, 97%, 98% or 99% identity) to a sequence of Table 2 or a portion of a sequence of Table 2. The direct repeat sequence can have at least 90% identity to a sequence comprising nucleotide 1 through nucleotide 36 of any one of SEQ ID NOs: 936, 937, 938, 939, 940, 941, 942, 943, 944, 945, 946, 947, 948, 949, 950, 951, 952, or 953. The direct repeat sequence can have at least 90% identity to a sequence comprising 2 through nucleotide 36 of any one of SEQ ID NOs: 936, 937, 938, 939, 940, 941, 942, 943, 944, 945, 946, 947, 948, 949, 950, 951, 952, or 953. The direct repeat sequence can have at least 90% identity to a sequence comprising 3 through nucleotide 36 of any one of SEQ ID NOs: 936, 937, 938, 939, 940, 941, 942, 943, 944, 945, 946, 947, 948, 949, 950, 951, 952, or 953. The direct repeat sequence can have at least 90% identity to a sequence comprising 4 through nucleotide 36 of any one of SEQ ID NOs: 936, 937, 938, 939, 940, 941, 942, 943, 944, 945, 946, 947, 948, 949, 950, 951, 952, or 953. The direct repeat sequence can have at least 90% identity to a sequence comprising 5 through nucleotide 36 of any one of SEQ ID NOs: 936, 937, 938, 939, 940, 941, 942, 943, 944, 945, 946, 947, 948, 949, 950, 951, 952, or 953. The direct repeat sequence can have at least 90% identity to a sequence comprising 6 through nucleotide 36 of any one of SEQ ID NOs: 936, 937, 938, 939, 940, 941, 942, 943, 944, 945, 946, 947, 948, 949, 950, 951, 952, or 953. The direct repeat sequence can have at least 90% identity to a sequence comprising 7 through nucleotide 36 of any one of SEQ ID NOs: 936, 937, 938, 939, 940, 941, 942, 943, 944, 945, 946, 947, 948, 949, 950, 951, 952, or 953. The direct repeat sequence can have at least 90% identity to a sequence comprising 8 through nucleotide 36 of any one of SEQ ID NOs: 936, 937, 938, 939, 940, 941, 942, 943, 944, 945, 946, 947, 948, 949, 950, 951, 952, or 953. The direct repeat sequence can have at least 90% identity to a sequence comprising 9 through nucleotide 36 of any one of SEQ ID NOs: 936, 937, 938, 939, 940, 941, 942, 943, 944, 945, 946, 947, 948, 949, 950, 951, 952, or 953. The direct repeat sequence can have at least 90% identity to a sequence comprising 10 through nucleotide 36 of any one of SEQ ID NOs: 936, 937, 938, 939, 940, 941, 942, 943, 944, 945, 946, 947, 948, 949, 950, 951, 952, or 953. The direct repeat sequence can have at least 90% identity to a sequence comprising 11 through nucleotide 36 of any one of SEQ ID NOs: 936, 937, 938, 939, 940, 941, 942, 943, 944, 945, 946, 947, 948, 949, 950, 951, 952, or 953. The direct repeat sequence can have at least 90% identity to a sequence comprising 12 through nucleotide 36 of any one of SEQ ID NOs: 936, 937, 938, 939, 940, 941, 942, 943, 944, 945, 946, 947, 948, 949, 950, 951, 952, or 953. The direct repeat sequence can have at least 90% identity to a sequence comprising 13 through nucleotide 36 of any one of SEQ ID NOs: 936, 937, 938, 939, 940, 941, 942, 943, 944, 945, 946, 947, 948, 949, 950, 951, 952, or 953.

In some embodiments, the direct repeat sequence is at least 90% identical to the reverse complement of any one of SEQ ID NOs: 936, 937, 938, 939, 940, 941, 942, 943, 944, 945, 946, 947, 948, 949, 950, 951, 952, or 953. In some embodiments, the direct repeat sequence is at least 95% identical to the reverse complement of any one of SEQ ID NOs: 936, 937, 938, 939, 940, 941, 942, 943, 944, 945, 946, 947, 948, 949, 950, 951, 952, or 953. In some embodiments, the direct repeat sequence is the reverse complement of any one of SEQ ID NOs: 936, 937, 938, 939, 940, 941, 942, 943, 944, 945, 946, 947, 948, 949, 950, 951, 952, or 953.

In some embodiments, the direct repeat sequence is at least 90% identical to SEQ ID NO: 954 or a portion of SEQ ID NO: 954. In some embodiments, the direct repeat sequence is at least 95% identical to SEQ ID NO: 954 or a portion of SEQ ID NO: 954. In some embodiments, the direct repeat sequence is 100% identical to SEQ ID NO: 954 or a portion of SEQ ID NO: 954.

TABLE 2
Cas12i4 Direct Repeat Sequences

| Sequence identifier | Direct Repeat Sequence |
|---|---|
| SEQ ID NO: 936 | UCUCAACGAUAGUCAGACAUGUGUCCUCAGUGACAC |
| SEQ ID NO: 937 | UUUUAACAACACUCAGGCAUGUGUCCACAGUGACAC |
| SEQ ID NO: 938 | UUGAACGGAUACUCAGACAUGUGUUUCCAGUGACAC |
| SEQ ID NO: 939 | UGCCCUCAAUAGUCAGAUGUGUGUCCACAGUGACAC |
| SEQ ID NO: 940 | UCUCAAUGAUACUUAGAUACGUGUCCUCAGUGACAC |
| SEQ ID NO: 941 | UCUCAAUGAUACUCAGACAUGUGUCCCCAGUGACAC |
| SEQ ID NO: 942 | UCUCAAUGAUACUAAGACAUGUGUCCUCAGUGACAC |
| SEQ ID NO: 943 | UCUCAACUAUACUCAGACAUGUGUCCUCAGUGACAC |
| SEQ ID NO: 944 | UCUCAACGAUACUCAGACAUGUGUCCUCAGUGACAC |
| SEQ ID NO: 945 | UCUCAACGAUACUAAGAUAUGUGUCCUCAGCGACAC |
| SEQ ID NO: 946 | UCUCAACGAUACUAAGAUAUGUGUCCCCAGUGACAC |
| SEQ ID NO: 947 | UCUCAACGAUACUAAGAUAUGUGUCCACAGUGACAC |
| SEQ ID NO: 948 | UCUCAACAAUACUCAGACAUGUGUCCCCAGUGACAC |
| SEQ ID NO: 949 | UCUCAACAAUACUAAGGCAUGUGUCCCCAGUGACCC |
| SEQ ID NO: 950 | UCUCAAAGAUACUCAGACACGUGUCCCCAGUGACAC |
| SEQ ID NO: 951 | UCUCAAAAAUACUCAGACAUGUGUCCUCAGUGACAC |
| SEQ ID NO: 952 | GCGAAACAACAGUCAGACAUGUGUCCCCAGUGACAC |
| SEQ ID NO: 953 | CCUCAACGAUAUUAAGACAUGUGUCCGCAGUGACAC |
| SEQ ID NO: 954 | AGACAUGUGUCCUCAGUGACAC |

In some embodiments, the direct repeat sequence is a sequence of Table 3 or a portion of a sequence of Table 3. In some embodiments, the direct repeat sequence has at least 95% identity (e.g., at least 95%, 96%, 97%, 98% or 99% identity) to a sequence of Table 3 or a portion of a sequence of Table 3. In some embodiments, the direct repeat sequence has at least 90% identity (e.g., at least 90%, 91%, 92%, 93%, 94%, 95%, 96%, 97%, 98% or 99% identity) to a sequence of Table 3 or a portion of a sequence of Table 3. In some embodiments, the direct repeat sequence is at least 90% identical to the reverse complement of any one of SEQ ID NOS: 959-961. In some embodiments, the direct repeat sequence is at least 95% identical to the reverse complement of any one of SEQ ID NOs: 959-961. In some embodiments, the direct repeat sequence is the reverse complement of any one of SEQ ID NOs: 959-961.

TABLE 3
Cas12i1 Direct Repeat Sequences

| Sequence | |
|---|---|
| identifier | Direct Repeat Sequence |
| SEQ ID NO: 959 | GUUGGAAUGACUAAUUUUUGUGCCCACCGUUGGC |
| | AC |
| SEQ ID NO: 960 | AAUUUUUGUGCCCAUCGUUGGCAC |
| SEQ ID NO: 961 | AUUUUUGUGCCCAUCGUUGGCAC |

In some embodiments, the direct repeat sequence is a sequence of Table 4 or a portion of a sequence of Table 4. In some embodiments, the direct repeat sequence has at least 95% identity (e.g., at least 95%, 96%, 97%, 98% or 99% identity) to a sequence of Table 4 or a portion of a sequence of Table 4. In some embodiments, the direct repeat sequence has at least 90% identity (e.g., at least 90%, 91%, 92%, 93%, 94%, 95%, 96%, 97%, 98% or 99% identity) to a sequence of Table 4 or a portion of a sequence of Table 4. In some embodiments, the direct repeat sequence is at least 90% identical to the reverse complement of any one of SEQ ID NOs: 962-964. In some embodiments, the direct repeat sequence is at least 95% identical to the reverse complement of any one of SEQ ID NOs: 962-964. In some embodiments, the direct repeat sequence is the reverse complement of any one of SEQ ID NOs: 962-964.

TABLE 4
Cas12i3 Direct Repeat Sequences

| Sequence | |
|---|---|
| identifier | Direct Repeat Sequence |
| SEQ ID NO: 962 | CUAGCAAUGACCUAAUAGUGUGUCCUUAGUUGAC |
| | AU |
| SEQ ID NO: 963 | CCUACAAUACCUAAGAAAUCCGUCCUAAGUUGAC |
| | GG |
| SEQ ID NO: 964 | AUAGUGUGUCCUUAGUUGACAU |

In some embodiments, a direct repeat sequence described herein comprises an uracil (U). In some embodiments, a direct repeat sequence described herein comprises a thymine (T). In some embodiments, a direct repeat sequence according to Tables 1-4 comprises a sequence comprising a thymine in one or more places indicated as uracil in Tables 1-4.

### (ii). Spacer Sequence

In some embodiments, the RNA guide comprises a DNA targeting or spacer sequence. In some embodiments, the spacer sequence of the RNA guide has a length of between 12-100, 13-75, 14-50, or 15-30 nucleotides (e.g., 15, 16, 17, 18, 19, 20, 21, 22, 23, 24, 25, 26, 27, 28, 29, or 30 nucleotides) and is complementary to a non-PAM strand sequence. In some embodiments, the spacer sequence is designed to be complementary to a specific DNA strand, e.g., of a genomic locus.

In some embodiments, the RNA guide spacer sequence is substantially identical to a complementary strand of a target sequence. In some embodiments, the RNA guide comprises a sequence (e.g., a spacer sequence) having at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to a complementary strand of a reference nucleic acid sequence, e.g., a target sequence. The percent identity between two such nucleic acids can be determined manually by inspection of the two optimally aligned nucleic acid sequences or by using software programs or algorithms (e.g., BLAST, ALIGN, CLUSTAL) using standard parameters.

In some embodiments, the RNA guide comprises a spacer sequence that has a length of between 12-100, 13-75, 14-50, or 15-30 nucleotides (e.g., 15, 16, 17, 18, 19, 20, 21, 22, 23, 24, 25, 26, 27, 28, 29, or 30 nucleotides) and at least 80%, at least 90%, at least 95%, at least 96%, at least 97%, at least 98%, at least 99% complementary to a region on the non-PAM strand that is complementary to the target sequence. In some embodiments, the RNA guide comprises a sequence at least 80%, at least 90%, at least 95%, at least 96%, at least 97%, at least 98%, at least 99% complementary to a target DNA sequence. In some embodiments, the RNA guide comprises a sequence at least 80%, at least 90%, at least 95%, at least 96%, at least 97%, at least 98%, at least 99% complementary to a target genomic sequence. In some embodiments, the RNA guide comprises a sequence, e.g., RNA sequence, that is a length of up to 50 and at least 80%, at least 90%, at least 95%, at least 96%, at least 97%, at least 98%, at least 99% complementary to a region on the non-PAM strand that is complementary to the target sequence. In some embodiments, the RNA guide comprises a sequence at least 80%, at least 90%, at least 95%, at least 96%, at least 97%, at least 98%, at least 99% complementary to a target DNA sequence. In some embodiments, the RNA guide comprises a sequence at least 80%, at least 90%, at least 95%, at least 96%, at least 97%, at least 98%, at least 99% complementary to a target genomic sequence.

In some embodiments, the spacer sequence is a sequence of Table 5 or a portion of a sequence of Table 5. It should be understood that an indication of SEQ ID NOs: 466-920 should be considered as equivalent to a listing of SEQ ID NOs: 466-920, with each of the intervening numbers present in the listing, i.e., 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, and 920.

The spacer sequence can comprise nucleotide 1 through nucleotide 16 of any one of SEQ ID NOs: 466-920. The spacer sequence can comprise nucleotide 1 through nucleotide 17 of any one of SEQ ID NOs: 466-920. The spacer sequence can comprise nucleotide 1 through nucleotide 18 of any one of SEQ ID NOs: 466-920. The spacer sequence can comprise nucleotide 1 through nucleotide 19 of any one of SEQ ID NOs: 466-920. The spacer sequence can comprise nucleotide 1 through nucleotide 20 of any one of SEQ ID NOs: 466-920. The spacer sequence can comprise nucleotide 1 through nucleotide 21 of any one of SEQ ID NOS: 466-920. The spacer sequence can comprise nucleotide 1 through nucleotide 22 of any one of SEQ ID NOs: 466-920. The spacer sequence can comprise nucleotide 1 through nucleotide 23 of any one of SEQ ID NOs: 466-920. The spacer sequence can comprise nucleotide 1 through nucleotide 24 of any one of SEQ ID NOs: 466-920. The spacer sequence can comprise nucleotide 1 through nucleotide 25 of any one of SEQ ID NOs: 466-920. The spacer sequence can comprise nucleotide 1 through nucleotide 26 of any one of SEQ ID NOs: 466-920. The spacer sequence can comprise nucleotide 1 through nucleotide 27 of any one of SEQ ID NOS: 466-920. The spacer sequence can comprise nucleotide 1 through nucleotide 28 of any one of SEQ ID NOs: 466-920. The spacer sequence can comprise nucleotide 1 through nucleotide 29 of any one of SEQ ID NOs: 466-920. The spacer sequence can comprise nucleotide 1 through nucleotide 30 of any one of SEQ ID NOs: 466-920.

In some embodiments, the spacer sequence has at least 90% identity (e.g., at least 90%, 91%, 92%, 93%, 94%, 95%, 96%, 97%, 98% or 99% identity) to a sequence of Table 5 or a portion of a sequence of Table 5. The spacer sequence can have at least 90% identity to a sequence comprising nucleotide 1 through nucleotide 16 of any one of SEQ ID NOs: 466-920. The spacer sequence can have at least 90% identity to a sequence comprising nucleotide 1 through nucleotide 17 of any one of SEQ ID NOs: 466-920. The spacer sequence can have at least 90% identity to a sequence comprising nucleotide 1 through nucleotide 18 of any one of SEQ ID NOs: 466-920. The spacer sequence can have at least 90% identity to a sequence comprising nucleotide 1 through nucleotide 19 of any one of SEQ ID NOs: 466-920. The spacer sequence can have at least 90% identity to a sequence comprising nucleotide 1 through nucleotide 20 of any one of SEQ ID NOs: 466-920. The spacer sequence can have at least 90% identity to a sequence comprising nucleotide 1 through nucleotide 21 of any one of SEQ ID NOS: 466-920. The spacer sequence can have at least 90% identity to a sequence comprising nucleotide 1 through nucleotide 22 of any one of SEQ ID NOs: 466-920. The spacer sequence can have at least 90% identity to a sequence comprising nucleotide 1 through nucleotide 23 of any one of SEQ ID NOs: 466-920. The spacer sequence can have at least 90% identity to a sequence comprising nucleotide 1 through nucleotide 24 of any one of SEQ ID NOs: 466-920.

The spacer sequence can have at least 90% identity to a sequence comprising nucleotide 1 through nucleotide 25 of any one of SEQ ID NOs: 466-920. The spacer sequence can have at least 90% identity to a sequence comprising nucleotide 1 through nucleotide 26 of any one of SEQ ID NOs: 466-920. The spacer sequence can have at least 90% identity to a sequence comprising nucleotide 1 through nucleotide 27 of any one of SEQ ID NOs: 466-920. The spacer sequence can have at least 90% identity to a sequence comprising nucleotide 1 through nucleotide 28 of any one of SEQ ID NOs: 466-920. The spacer sequence can have at least 90% identity to a sequence comprising nucleotide 1 through nucleotide 29 of any one of SEQ ID NOs: 466-920. The spacer sequence can have at least 90% identity to a sequence comprising nucleotide 1 through nucleotide 30 of any one of 466-920.

TABLE 5
Target and Spacer Sequences

| | | | SEQ ID | | SEQ ID | |
|---|---|---|---|---|---|---|
| HAO1 | Strand | PAM* | NO | Target Sequence | NO | Spacer Sequence |
| HAO1_exon1 | + | CTTA | 11 | CCTGGAAAATGCTGCAA | 466 | CCUGGAAAAUGCUGCAAUA |
| | | | | TATTATCAGCCAA | | UUAUCAGCCAA |
| HAO1_exon1 | + | ATTT | 12 | TCTTACCTGGAAAATGC | 467 | UCUUACCUGGAAAAUGCUG |
| | | | | TGCAATATTATCA | | CAAUAUUAUCA |
| HAO1_exon1 | + | TTTT | 13 | CTTACCTGGAAAATGCT | 468 | CUUACCUGGAAAAUGCUGC |
| | | | | GCAATATTATCAG | | AAUAUUAUCAG |
| HAO1_exon1 | + | TTTC | 14 | TTACCTGGAAAATGCTG | 469 | UUACCUGGAAAAUGCUGCA |
| | | | | CAATATTATCAGC | | AUAUUAUCAGC |
| HAO1_exon1 | + | ATTA | 15 | TCAGCCAAAGTTTCTTC | 470 | UCAGCCAAAGUUUCUUCAU |
| | | | | ATCATTTGCCCCA | | CAUUUGCCCCA |
| HAO1_exon1 | + | GTTT | 16 | CTTCATCATTTGCCCCA | 471 | CUUCAUCAUUUGCCCCAGA |
| | | | | GACCTGTAATAGT | | CCUGUAAUAGU |
| HAO1_exon1 | + | TTTC | 17 | TTCATCATTTGCCCCAG | 472 | UUCAUCAUUUGCCCCAGAC |
| | | | | ACCTGTAATAGTC | | CUGUAAUAGUC |
| HAO1_exon1 | + | CTTC | 18 | ATCATTTGCCCCAGACC | 473 | AUCAUUUGCCCCAGACCUG |
| | | | | TGTAATAGTCATA | | UAAUAGUCAUA |
| HAO1_exon1 | + | ATTT | 19 | GCCCCAGACCTGTAATA | 474 | GCCCCAGACCUGUAAUAGU |
| | | | | GTCATATATAGAC | | CAUAUAUAGAC |
| HAO1_exon1 | + | TTTG | 20 | CCCCAGACCTGTAATAG | 475 | CCCCAGACCUGUAAUAGUC |
| | | | | TCATATATAGACT | | AUAUAUAGACU |
| HAO1_exon1 | + | TTTT | 21 | AAAAAATAAATTTTCTT | 476 | AAAAAAUAAAUUUUCUUAC |
| | | | | ACCTGGAAAATGC | | CUGGAAAAUGC |
| HAO1_exon1 | + | CTTT | 22 | GGAAGTACTGATTTAGC | 477 | GGAAGUACUGAUUUAGCAU |
| | | | | ATGTTGTTCATAA | | GUUGUUCAUAA |
| HAO1_exon1 | + | ATTT | 23 | AGCATGTTGTTCATAAT | 478 | AGCAUGUUGUUCAUAAUCA |
| | | | | CATTGATACAAAT | | UUGAUACAAAU |
| HAO1_exon1 | + | TTTA | 24 | GCATGTTGTTCATAATC | 479 | GCAUGUUGUUCAUAAUCAU |
| | | | | ATTGATACAAATT | | UGAUACAAAUU |
| HAO1_exon1 | + | GTTG | 25 | TTCATAATCATTGATAC | 480 | UUCAUAAUCAUUGAUACAA |
| | | | | AAATTAGCCGGGG | | AUUAGCCGGGG |
| HAO1_exon1 | + | GTTC | 26 | ATAATCATTGATACAAA | 481 | AUAAUCAUUGAUACAAAUU |
| | | | | TTAGCCGGGGGAG | | AGCCGGGGGAG |
| HAO1_exon1 | + | ATTG | 27 | ATACAAATTAGCCGGGG | 482 | AUACAAAUUAGCCGGGGGA |
| | | | | GAGCATTTTCACA | | GCAUUUUCACA |
| HAO1_exon1 | + | ATTA | 28 | GCCGGGGGAGCATTTTC | 483 | GCCGGGGGAGCAUUUUCAC |
| | | | | ACAGGTTATTGCT | | AGGUUAUUGCU |
| HAO1_exon1 | + | ATTT | 29 | TCACAGGTTATTGCTAT | 484 | UCACAGGUUAUUGCUAUCC |
| | | | | CCCAGATGGAGTT | | CAGAUGGAGUU |
| HAO1_exon1 | + | TTTT | 30 | CACAGGTTATTGCTATC | 485 | CACAGGUUAUUGCUAUCCC |
| | | | | CCAGATGGAGTTC | | AGAUGGAGUUC |
| HAO1_exon1 | + | TTTC | 31 | ACAGGTTATTGCTATCC | 486 | ACAGGUUAUUGCUAUCCCA |
| | | | | CAGATGGAGTTCG | | GAUGGAGUUCG |
| HAO1_exon1 | + | TTTG | 32 | GAAGTACTGATTTAGCA | 487 | GAAGUACUGAUUUAGCAUG |
| | | | | TGTTGTTCATAAT | | UUGUUCAUAAU |
| HAO1_exon1 | + | ATTT | 33 | TAAAAAATAAATTTTCT | 488 | UAAAAAAUAAAUUUUCUUA |
| | | | | TACCTGGAAAATG | | CCUGGAAAAUG |
| HAO1_exon1 | + | TTTA | 34 | AAAAATAAATTTTCTTA | 489 | AAAAAUAAAUUUUCUUACC |
| | | | | CCTGGAAAATGCT | | UGGAAAAUGCU |
| HAO1_exon1 | + | TTTT | 35 | AAAACATGATTTTAAAA | 490 | AAAACAUGAUUUUAAAAAA |
| | | | | AATAAATTTTCTT | | UAAAUUUUCUU |
| HAO1_exon1 | − | TTTG | 36 | TATCAATGATTATGAAC | 491 | UAUCAAUGAUUAUGAACAA |
| | | | | AACATGCTAAATC | | CAUGCUAAAUC |
| HAO1_exon1 | − | ATTA | 37 | TGAACAACATGCTAAAT | 492 | UGAACAACAUGCUAAAUCA |
| | | | | CAGTACTTCCAAA | | GUACUUCCAAA |
| HAO1_exon1 | − | CTTC | 38 | CAAAGTCTATATATGAC | 493 | CAAAGUCUAUAUAUGACUA |
| | | | | TATTACAGGTCTG | | UUACAGGUCUG |
| HAO1_exon1 | − | ATTA | 39 | CAGGTCTGGGGCAAATG | 494 | CAGGUCUGGGGCAAAUGAU |
| | | | | ATGAAGAAACTTT | | GAAGAAACUUU |
| HAO1_exon1 | − | CTTT | 40 | GGCTGATAATATTGCAG | 495 | GGCUGAUAAUAUUGCAGCA |
| | | | | CATTTTCCAGGTA | | UUUUCCAGGUA |
| HAO1_exon1 | − | TTTG | 41 | GCTGATAATATTGCAGC | 496 | GCUGAUAAUAUUGCAGCAU |
| | | | | ATTTTCCAGGTAA | | UUUCCAGGUAA |
| HAO1_exon1 | − | ATTG | 42 | CAGCATTTTCCAGGTAA | 497 | CAGCAUUUUCCAGGUAAGA |
| | | | | GAAAATTTATTTT | | AAAUUUAUUUU |
| HAO1_exon1 | − | ATTT | 43 | TCCAGGTAAGAAAATTT | 498 | UCCAGGUAAGAAAAUUUAU |
| | | | | ATTTTTTAAAATC | | UUUUUAAAAUC |
| HAO1_exon1 | − | TTTT | 44 | CCAGGTAAGAAAATTTA | 499 | CCAGGUAAGAAAAUUUAUU |
| | | | | TTTTTTAAAATCA | | UUUUAAAAUCA |
| HAO1_exon1 | + | TTTA | 45 | AAACATGATTTTAAAAA | 500 | AAACAUGAUUUUAAAAAAU |
| | | | | ATAAATTTTCTTA | | AAAUUUUCUUA |
| HAO1_exon1 | − | ATTT | 46 | ATTTTTTAAAATCATGT | 501 | AUUUUUUAAAAUCAUGUUU |
| | | | | TTTAAAATTACAC | | UAAAAUUACAC |
| HAO1_exon1 | − | TTTC | 47 | CAGGTAAGAAAATTTAT | 502 | CAGGUAAGAAAAUUUAUUU |
| | | | | TTTTTAAAATCAT | | UUUAAAAUCAU |
| HAO1_exon1 | − | ATTT | 48 | TTTAAAATCATGTTTTA | 503 | UUUAAAAUCAUGUUUUAAA |
| | | | | AAATTACACAAAG | | AUUACACAAAG |
| HAO1_exon1 | − | TTTT | 49 | TTAAAATCATGTTTTAA | 504 | UUAAAAUCAUGUUUUAAAA |
| | | | | AATTACACAAAGA | | UUACACAAAGA |
| HAO1_exon1 | − | TTTT | 50 | TAAAATCATGTTTTAAA | 505 | UAAAAUCAUGUUUUAAAAU |
| | | | | ATTACACAAAGAC | | UACACAAAGAC |
| HAO1_exon1 | − | TTTT | 51 | AAAATCATGTTTTAAAA | 506 | AAAAUCAUGUUUUAAAAUU |
| | | | | TTACACAAAGACC | | ACACAAAGACC |
| HAO1_exon1 | − | TTTA | 52 | AAATCATGTTTTAAAAT | 507 | AAAUCAUGUUUUAAAAUUA |
| | | | | TACACAAAGACCG | | CACAAAGACCG |
| HAO1_exon1 | + | CTTT | 53 | GTGTAATTTTAAAACAT | 508 | GUGUAAUUUUAAAACAUGA |
| | | | | GATTTTAAAAAAT | | UUUUAAAAAAU |
| HAO1_exon1 | + | TTTG | 54 | TGTAATTTTAAAACATG | 509 | UGUAAUUUUAAAACAUGAU |
| | | | | ATTTTAAAAAATA | | UUUAAAAAAUA |
| HAO1_exon1 | + | ATTT | 55 | TAAAACATGATTTTAAA | 510 | UAAAACAUGAUUUUAAAAA |
| | | | | AAATAAATTTTCT | | AUAAAUUUUCU |
| HAO1_exon1 | − | TTTA | 56 | TTTTTTAAAATCATGTT | 511 | UUUUUUAAAAUCAUGUUUU |
| | | | | TTAAAATTACACA | | AAAAUUACACA |
| HAO1_exon1 | − | ATTT | 57 | GTATCAATGATTATGAA | 512 | GUAUCAAUGAUUAUGAACA |
| | | | | CAACATGCTAAAT | | ACAUGCUAAAU |
| HAO1_exon1 | + | GTTA | 58 | TTGCTATCCCAGATGGA | 513 | UUGCUAUCCCAGAUGGAGU |
| | | | | GTTCGTT | | UCGUU |
| HAO1_exon1 | + | ATTG | 59 | CTATCCCAGATGGAGTT | 514 | CUAUCCCAGAUGGAGUUCG |
| | | | | CGTT | | UU |
| HAO1_exon2 | − | TTTA | 60 | TTTTTTAATTCTAGATG | 515 | UUUUUUAAUUCUAGAUGGA |
| | | | | GAAGCTGTATCCA | | AGCUGUAUCCA |
| HAO1_exon2 | − | TTTT | 61 | ATTTTATTTTTTAATTC | 516 | AUUUUAUUUUUUAAUUCUA |
| | | | | TAGATGGAAGCTG | | GAUGGAAGCUG |
| HAO1_exon2 | − | TTTT | 62 | ATTTTTTAATTCTAGAT | 517 | AUUUUUUAAUUCUAGAUGG |
| | | | | GGAAGCTGTATCC | | AAGCUGUAUCC |
| HAO1_exon2 | − | ATTT | 63 | TATTTTTTAATTCTAGA | 518 | UAUUUUUUAAUUCUAGAUG |
| | | | | TGGAAGCTGTATC | | GAAGCUGUAUC |
| HAO1_exon2 | − | TTTA | 64 | TTTTATTTTTTAATTCT | 519 | UUUUAUUUUUUAAUUCUAG |
| | | | | AGATGGAAGCTGT | | AUGGAAGCUGU |
| HAO1_exon2 | + | ATTA | 65 | AAAAATAAAATAAAATA | 520 | AAAAAUAAAAUAAAAUAAA |
| | | | | AAAGGCTTTAGAG | | AGGCUUUAGAG |
| HAO1_exon2 | − | TTTT | 66 | ATTTTATTTTATTTTTT | 521 | AUUUUAUUUUAUUUUUUAA |
| | | | | AATTCTAGATGGA | | UUCUAGAUGGA |
| HAO1_exon2 | − | CTTT | 67 | TATTTTATTTTATTTTT | 522 | UAUUUUAUUUUAUUUUUUA |
| | | | | TAATTCTAGATGG | | AUUCUAGAUGG |
| HAO1_exon2 | − | ATTC | 68 | TGAAACTCTAAAGCCTT | 523 | UGAAACUCUAAAGCCUUUU |
| | | | | TTATTTTATTTTA | | AUUUUAUUUUA |
| HAO1_exon2 | − | ATTT | 69 | TTTAATTCTAGATGGAA | 524 | UUUAAUUCUAGAUGGAAGC |
| | | | | GCTGTATCCAAGG | | UGUAUCCAAGG |
| HAO1_exon2 | − | TTTA | 70 | TTTTATTTTATTTTTTA | 525 | UUUUAUUUUAUUUUUUAAU |
| | | | | ATTCTAGATGGAA | | UCUAGAUGGAA |
| HAO1_exon2 | − | TTTT | 71 | TTAATTCTAGATGGAAG | 526 | UUAAUUCUAGAUGGAAGCU |
| | | | | CTGTATCCAAGGA | | GUAUCCAAGGA |
| HAO1_exon2 | − | ATTT | 72 | TATTTTATTTTTTAATT | 527 | UAUUUUAUUUUUUAAUUCU |
| | | | | CTAGATGGAAGCT | | AGAUGGAAGCU |
| HAO1_exon2 | + | CTTC | 73 | CATCTAGAATTAAAAAA | 528 | CAUCUAGAAUUAAAAAAUA |
| | | | | TAAAATAAAATAA | | AAAUAAAAUAA |
| HAO1_exon2 | − | TTTT | 74 | TAATTCTAGATGGAAGC | 529 | UAAUUCUAGAUGGAAGCUG |
| | | | | TGTATCCAAGGAT | | UAUCCAAGGAU |
| HAO1_exon2 | − | TTTT | 75 | AATTCTAGATGGAAGCT | 530 | AAUUCUAGAUGGAAGCUGU |
| | | | | GTATCCAAGGATG | | AUCCAAGGAUG |
| HAO1_exon2 | − | TTTA | 76 | ATTCTAGATGGAAGCTG | 531 | AUUCUAGAUGGAAGCUGUA |
| | | | | TATCCAAGGATGC | | UCCAAGGAUGC |
| HAO1_exon2 | − | ATTC | 77 | TAGATGGAAGCTGTATC | 532 | UAGAUGGAAGCUGUAUCCA |
| | | | | CAAGGATGCTCCG | | AGGAUGCUCCG |
| HAO1_exon2 | − | GTTG | 78 | CTGAAACAGATCTGTCG | 533 | CUGAAACAGAUCUGUCGAC |
| | | | | ACTTCTGTTTTAG | | UUCUGUUUUAG |
| HAO1_exon2 | − | GTTT | 79 | TAGGACAGAGGGTCAGC | 534 | UAGGACAGAGGGUCAGCAU |
| | | | | ATGCCAATATGTG | | GCCAAUAUGUG |
| HAO1_exon2 | − | TTTT | 80 | AGGACAGAGGGTCAGCA | 535 | AGGACAGAGGGUCAGCAUG |
| | | | | TGCCAATATGTGT | | CCAAUAUGUGU |
| HAO1_exon2 | − | TTTA | 81 | GGACAGAGGGTCAGCAT | 536 | GGACAGAGGGUCAGCAUGC |
| | | | | GCCAATATGTGTG | | CAAUAUGUGUG |
| HAO1_exon2 | − | CTTG | 82 | CCACTGTGAGAGGTAGG | 537 | CCACUGUGAGAGGUAGGAG |
| | | | | AGGAAGATTGTCA | | GAAGAUUGUCA |
| HAO1_exon2 | − | CTTC | 83 | TGTTTTAGGACAGAGGG | 538 | UGUUUUAGGACAGAGGGUC |
| | | | | TCAGCATGCCAAT | | AGCAUGCCAAU |
| HAO1_exon2 | + | GTTA | 84 | GCCTCCTTCTGTCCCTG | 539 | GCCUCCUUCUGUCCCUGUG |
| | | | | TGGTGACAATCTT | | GUGACAAUCUU |
| HAO1_exon2 | − | ATTG | 85 | TCACCACAGGGACAGAA | 540 | UCACCACAGGGACAGAAGG |
| | | | | GGAGGCTAACGTT | | AGGCUAACGUU |
| HAO1_exon2 | + | ATTC | 86 | CGGAGCATCCTTGGATA | 541 | CGGAGCAUCCUUGGAUACA |
| | | | | CAGCTTCCATCTA | | GCUUCCAUCUA |
| HAO1_exon2 | + | TTTC | 87 | AGCAACATTCCGGAGCA | 542 | AGCAACAUUCCGGAGCAUC |
| | | | | TCCTTGGATACAG | | CUUGGAUACAG |
| HAO1_exon2 | + | GTTT | 88 | CAGCAACATTCCGGAGC | 543 | CAGCAACAUUCCGGAGCAU |
| | | | | ATCCTTGGATACA | | CCUUGGAUACA |
| HAO1_exon2 | + | CTTG | 89 | GATACAGCTTCCATCTA | 544 | GAUACAGCUUCCAUCUAGA |
| | | | | GAATTAAAAAATA | | AUUAAAAAAUA |
| HAO1_exon2 | + | CTTC | 90 | CTCCTACCTCTCACAGT | 545 | CUCCUACCUCUCACAGUGG |
| | | | | GGCAAGCTCGCCG | | CAAGCUCGCCG |
| HAO1_exon2 | + | CTTC | 91 | TGTCCCTGTGGTGACAA | 546 | UGUCCCUGUGGUGACAAUC |
| | | | | TCTTCCTCCTACC | | UUCCUCCUACC |
| HAO1_exon2 | + | ATTG | 92 | GCATGCTGACCCTCTGT | 547 | GCAUGCUGACCCUCUGUCC |
| | | | | CCTAAAACAGAAG | | UAAAACAGAAG |
| HAO1_exon3 | − | CTTA | 93 | CCTGGGCAACCGTCTGG | 548 | CCUGGGCAACCGUCUGGAU |
| | | | | ATGATGTGCGTAA | | GAUGUGCGUAA |
| HAO1_exon3 | + | TTTG | 94 | AATCTGTTACGCACATC | 549 | AAUCUGUUACGCACAUCAU |
| | | | | ATCCAGACGGTTG | | CCAGACGGUUG |
| HAO1_exon3 | + | GTTT | 95 | GAATCTGTTACGCACAT | 550 | GAAUCUGUUACGCACAUCA |
| | | | | CATCCAGACGGTT | | UCCAGACGGUU |
| HAO1_exon3 | + | GTTG | 96 | TGGCGGCAGTTTGAATC | 551 | UGGCGGCAGUUUGAAUCUG |
| | | | | TGTTACGCACATC | | UUACGCACAUC |
| HAO1_exon3 | + | GTTA | 97 | CCTGAGTTGTGGCGGCA | 552 | CCUGAGUUGUGGCGGCAGU |
| | | | | GTTTGAATCTGTT | | UUGAAUCUGUU |
| HAO1_exon3 | + | TTTC | 98 | GCCTCAGCTCGGGGCCC | 553 | GCCUCAGCUCGGGGCCCAC |
| | | | | ACATGATCATGGT | | AUGAUCAUGGU |
| HAO1_exon3 | + | CTTT | 99 | CGCCTCAGCTCGGGGCC | 554 | CGCCUCAGCUCGGGGCCCA |
| | | | | CACATGATCATGG | | CAUGAUCAUGG |
| HAO1_exon3 | − | ATTC | 100 | AAACTGCCGCCACAACT | 555 | AAACUGCCGCCACAACUCA |
| | | | | CAGGTAACCATGA | | GGUAACCAUGA |
| HAO1_exon3 | − | TTTG | 101 | TGACAGTGGACACACCT | 556 | UGACAGUGGACACACCUUA |
| | | | | TACCTGGGCAACC | | CCUGGGCAACC |
| HAO1_exon3 | − | CTTG | 102 | ATCATCCCCTTTCTTTC | 557 | AUCAUCCCCUUUCUUUCUC |
| | | | | TCAGCCTGTCAGT | | AGCCUGUCAGU |
| HAO1_exon3 | − | GTTG | 103 | GCTGCAACTGTATATCT | 558 | GCUGCAACUGUAUAUCUAC |
| | | | | ACAAGGACCGAGA | | AAGGACCGAGA |
| HAO1_exon3 | − | ATTG | 104 | AAGAAGTGGCGGAAGCT | 559 | AAGAAGUGGCGGAAGCUGG |
| | | | | GGTCCTGAGGCAC | | UCCUGAGGCAC |
| HAO1_exon3 | − | GTTC | 105 | CTGGGCCACCTCCTCAA | 560 | CUGGGCCACCUCCUCAAUU |
| | | | | TTGAAGAAGTGGC | | GAAGAAGUGGC |
| HAO1_exon3 | − | GTTG | 106 | AGTTCCTGGGCCACCTC | 561 | AGUUCCUGGGCCACCUCCU |
| | | | | CTCAATTGAAGAA | | CAAUUGAAGAA |
| HAO1_exon3 | − | TTTC | 107 | TCAGCCTGTCAGTCCCT | 562 | UCAGCCUGUCAGUCCCUGG |
| | | | | GGGAACGGGCATG | | GAACGGGCAUG |
| HAO1_exon3 | − | CTTT | 108 | CTCAGCCTGTCAGTCCC | 563 | CUCAGCCUGUCAGUCCCUG |
| | | | | TGGGAACGGGCAT | | GGAACGGGCAU |
| HAO1_exon3 | − | TTTC | 109 | TTTCTCAGCCTGTCAGT | 564 | UUUCUCAGCCUGUCAGUCC |
| | | | | CCCTGGGAACGGG | | CUGGGAACGGG |
| HAO1_exon3 | − | CTTT | 110 | CTTTCTCAGCCTGTCAG | 565 | CUUUCUCAGCCUGUCAGUC |
| | | | | TCCCTGGGAACGG | | CCUGGGAACGG |
| HAO1_exon3 | + | GTTA | 111 | CGCACATCATCCAGACG | 566 | CGCACAUCAUCCAGACGGU |
| | | | | GTTGCCCAGGTAA | | UGCCCAGGUAA |
| HAO1_exon3 | − | ATTT | 112 | GTGACAGTGGACACACC | 567 | GUGACAGUGGACACACCUU |
| | | | | TTACCTGGGCAAC | | ACCUGGGCAAC |
| HAO1_exon3 | + | GTTG | 113 | CCCAGGTAAGGTGTGTC | 568 | CCCAGGUAAGGUGUGUCCA |
| | | | | CACTGTCACAAAT | | CUGUCACAAAU |
| HAO1_exon3 | − | CTTC | 114 | GTTGGCTGCAACTGTAT | 569 | GUUGGCUGCAACUGUAUAU |
| | | | | ATCTACAAGGACC | | CUACAAGGACC |
| HAO1_exon3 | + | CTTC | 115 | TCTGCCTGCCGCACTAG | 570 | UCUGCCUGCCGCACUAGCU |
| | | | | CTTCTTGGTGACT | | UCUUGGUGACU |
| HAO1_exon3 | + | CTTG | 116 | TAGCCCATCTTCTCTGC | 571 | UAGCCCAUCUUCUCUGCCU |
| | | | | CTGCCGCACTAGC | | GCCGCACUAGC |
| HAO1_exon3 | + | GTTC | 117 | CCAGGGACTGACAGGCT | 572 | CCAGGGACUGACAGGCUGA |
| | | | | GAGAAAGAAAGGG | | GAAAGAAAGGG |
| HAO1_exon3 | + | ATTG | 118 | AGGAGGTGGCCCAGGAA | 573 | AGGAGGUGGCCCAGGAACU |
| | | | | CTCAACATCATGC | | CAACAUCAUGC |
| HAO1_exon3 | + | CTTC | 119 | TTCAATTGAGGAGGTGG | 574 | UUCAAUUGAGGAGGUGGCC |
| | | | | CCCAGGAACTCAA | | CAGGAACUCAA |
| HAO1_exon3 | + | CTTC | 120 | CGCCACTTCTTCAATTG | 575 | CGCCACUUCUUCAAUUGAG |
| | | | | AGGAGGTGGCCCA | | GAGGUGGCCCA |
| HAO1_exon3 | + | CTTC | 121 | AATTGAGGAGGTGGCCC | 576 | AAUUGAGGAGGUGGCCCAG |
| | | | | AGGAACTCAACAT | | GAACUCAACAU |
| HAO1_exon3 | + | CTTG | 122 | TAGATATACAGTTGCAG | 577 | UAGAUAUACAGUUGCAGCC |
| | | | | CCAACGAAGTGCC | | AACGAAGUGCC |
| HAO1_exon3 | + | CTTC | 123 | TCGGTCCTTGTAGATAT | 578 | UCGGUCCUUGUAGAUAUAC |
| | | | | ACAGTTGCAGCCA | | AGUUGCAGCCA |
| HAO1_exon3 | + | CTTG | 124 | GTGACTTCTCGGTCCTT | 579 | GUGACUUCUCGGUCCUUGU |
| | | | | GTAGATATACAGT | | AGAUAUACAGU |
| HAO1_exon3 | + | CTTC | 125 | TTGGTGACTTCTCGGTC | 580 | UUGGUGACUUCUCGGUCCU |
| | | | | CTTGTAGATATAC | | UGUAGAUAUAC |
| HAO1_exon3 | + | GTTG | 126 | CAGCCAACGAAGTGCCT | 581 | CAGCCAACGAAGUGCCUCA |
| | | | | CAGGACCAGCTTC | | GGACCAGCUUC |
| HAO1_exon4 | − | ATTT | 127 | CTAATTTGGCAAATTTC | 582 | CUAAUUUGGCAAAUUUCUC |
| | | | | TCATTTTATGCAT | | AUUUUAUGCAU |
| HAO1_exon4 | + | TTTC | 128 | ATCCTAAAATAAGAAAT | 583 | AUCCUAAAAUAAGAAAUGC |
| | | | | GCATAAAATGAGA | | AUAAAAUGAGA |
| HAO1_exon4 | + | ATTC | 129 | AAGTAGAGAAATAAACG | 584 | AAGUAGAGAAAUAAACGAA |
| | | | | AACCTCTCAAAAT | | CCUCUCAAAAU |
| HAO1_exon4 | − | TTTC | 130 | TCTACTTGAATTCATAC | 585 | UCUACUUGAAUUCAUACUG |
| | | | | TGACTTTGTGATC | | ACUUUGUGAUC |
| HAO1_exon4 | − | TTTC | 131 | TAATTTGGCAAATTTCT | 586 | UAAUUUGGCAAAUUUCUCA |
| | | | | CATTTTATGCATT | | UUUUAUGCAUU |
| HAO1_exon4 | − | ATTT | 132 | TATGCATTTCTTATTTT | 587 | UAUGCAUUUCUUAUUUUAG |
| | | | | AGGATGAAAAATT | | GAUGAAAAAUU |
| HAO1_exon4 | − | TTTG | 133 | GCAAATTTCTCATTTTA | 588 | GCAAAUUUCUCAUUUUAUG |
| | | | | TGCATTTCTTATT | | CAUUUCUUAUU |
| HAO1_exon4 | − | ATTT | 134 | CTCATTTTATGCATTTC | 589 | CUCAUUUUAUGCAUUUCUU |
| | | | | TTATTTTAGGATG | | AUUUUAGGAUG |
| HAO1_exon4 | − | TTTC | 135 | TCATTTTATGCATTTCT | 590 | UCAUUUUAUGCAUUUCUUA |
| | | | | TATTTTAGGATGA | | UUUUAGGAUGA |
| HAO1_exon4 | + | TTTT | 136 | CATCCTAAAATAAGAAA | 591 | CAUCCUAAAAUAAGAAAUG |
| | | | | TGCATAAAATGAG | | CAUAAAAUGAG |
| HAO1_exon4 | − | ATTT | 137 | GGCAAATTTCTCATTTT | 592 | GGCAAAUUUCUCAUUUUAU |
| | | | | ATGCATTTCTTAT | | GCAUUUCUUAU |
| HAO1_exon4 | + | TTTT | 138 | TCATCCTAAAATAAGAA | 593 | UCAUCCUAAAAUAAGAAAU |
| | | | | ATGCATAAAATGA | | GCAUAAAAUGA |
| HAO1_exon4 | + | ATTT | 139 | TCCTCAGGAGAAAATGA | 594 | UCCUCAGGAGAAAAUGAUA |
| | | | | TAAAGTACTGGTT | | AAGUACUGGUU |
| HAO1_exon4 | + | TTTC | 140 | AAAATTTTTCATCCTAA | 595 | AAAAUUUUUCAUCCUAAAA |
| | | | | AATAAGAAATGCA | | UAAGAAAUGCA |
| HAO1_exon4 | + | GTTT | 141 | CAAAATTTTTCATCCTA | 596 | CAAAAUUUUUCAUCCUAAA |
| | | | | AAATAAGAAATGC | | AUAAGAAAUGC |
| HAO1_exon4 | + | TTTC | 142 | CTCAGGAGAAAATGATA | 597 | CUCAGGAGAAAAUGAUAAA |
| | | | | AAGTACTGGTTTC | | GUACUGGUUUC |
| HAO1_exon4 | + | TTTT | 143 | CCTCAGGAGAAAATGAT | 598 | CCUCAGGAGAAAAUGAUAA |
| | | | | AAAGTACTGGTTT | | AGUACUGGUUU |
| HAO1_exon4 | − | TTTT | 144 | ATGCATTTCTTATTTTA | 599 | AUGCAUUUCUUAUUUUAGG |
| | | | | GGATGAAAAATTT | | AUGAAAAAUUU |
| HAO1_exon4 | + | TTTA | 145 | GCCACATATGCAGCAAG | 600 | GCCACAUAUGCAGCAAGUC |
| | | | | TCCACTGTCGTCT | | CACUGUCGUCU |
| HAO1_exon4 | + | CTTT | 146 | AGCCACATATGCAGCAA | 601 | AGCCACAUAUGCAGCAAGU |
| | | | | GTCCACTGTCGTC | | CCACUGUCGUC |
| HAO1_exon4 | + | ATTG | 147 | CTTTAGCCACATATGCA | 602 | CUUUAGCCACAUAUGCAGC |
| | | | | GCAAGTCCACTGT | | AAGUCCACUGU |
| HAO1_exon4 | + | CTTC | 148 | CCAGCTGATAGATGGGT | 603 | CCAGCUGAUAGAUGGGUCU |
| | | | | CTATTGCTTTAGC | | AUUGCUUUAGC |
| HAO1_exon4 | + | TTTG | 149 | ATATCTTCCCAGCTGAT | 604 | AUAUCUUCCCAGCUGAUAG |
| | | | | AGATGGGTCTATT | | AUGGGUCUAUU |
| HAO1_exon4 | + | ATTT | 150 | GATATCTTCCCAGCTGA | 605 | GAUAUCUUCCCAGCUGAUA |
| | | | | TAGATGGGTCTAT | | GAUGGGUCUAU |
| HAO1_exon4 | + | CTTC | 151 | TCAGCCATTTGATATCT | 606 | UCAGCCAUUUGAUAUCUUC |
| | | | | TCCCAGCTGATAG | | CCAGCUGAUAG |
| HAO1_exon4 | + | ATTG | 152 | GCAATGATGTCAGTCTT | 607 | GCAAUGAUGUCAGUCUUCU |
| | | | | CTCAGCCATTTGA | | CAGCCAUUUGA |
| HAO1_exon4 | + | ATTT | 153 | TTCATCCTAAAATAAGA | 608 | UUCAUCCUAAAAUAAGAAA |
| | | | | AATGCATAAAATG | | UGCAUAAAAUG |
| HAO1_exon4 | − | TTTA | 154 | TGCATTTCTTATTTTAG | 609 | UGCAUUUCUUAUUUUAGGA |
| | | | | GATGAAAAATTTT | | UGAAAAAUUUU |
| HAO1_exon4 | − | TTTA | 155 | GGATGAAAAATTTTGAA | 610 | GGAUGAAAAAUUUUGAAAC |
| | | | | ACCAGTACTTTAT | | CAGUACUUUAU |
| HAO1_exon4 | − | TTTC | 156 | TTATTTTAGGATGAAAA | 611 | UUAUUUUAGGAUGAAAAAU |
| | | | | ATTTTGAAACCAG | | UUUGAAACCAG |
| HAO1_exon4 | − | ATTG | 157 | CCAATTGTTGCAAAGGG | 612 | CCAAUUGUUGCAAAGGGCA |
| | | | | CATTTTGAGAGGT | | UUUUGAGAGGU |
| HAO1_exon4 | − | ATTG | 158 | TTGCAAAGGGCATTTTG | 613 | UUGCAAAGGGCAUUUUGAG |
| | | | | AGAGGTTCGTTTA | | AGGUUCGUUUA |
| HAO1_exon4 | + | CTTT | 159 | GCAACAATTGGCAATGA | 614 | GCAACAAUUGGCAAUGAUG |
| | | | | TGTCAGTCTTCTC | | UCAGUCUUCUC |
| HAO1_exon4 | − | GTTG | 160 | CAAAGGGCATTTTGAGA | 615 | CAAAGGGCAUUUUGAGAGG |
| | | | | GGTTCGTTTATTT | | UUCGUUUAUUU |
| HAO1_exon4 | − | ATTT | 161 | TGAGAGGTTCGTTTATT | 616 | UGAGAGGUUCGUUUAUUUC |
| | | | | TCTCTACTTGAAT | | UCUACUUGAAU |
| HAO1_exon4 | − | TTTT | 162 | GAGAGGTTCGTTTATTT | 617 | GAGAGGUUCGUUUAUUUCU |
| | | | | CTCTACTTGAATT | | CUACUUGAAUU |
| HAO1_exon4 | − | TTTG | 163 | AGAGGTTCGTTTATTTC | 618 | AGAGGUUCGUUUAUUUCUC |
| | | | | TCTACTTGAATTC | | UACUUGAAUUC |
| HAO1_exon4 | − | GTTC | 164 | GTTTATTTCTCTACTTG | 619 | GUUUAUUUCUCUACUUGAA |
| | | | | AATTCATACTGAC | | UUCAUACUGAC |
| HAO1_exon4 | − | GTTT | 165 | ATTTCTCTACTTGAATT | 620 | AUUUCUCUACUUGAAUUCA |
| | | | | CATACTGACTTTG | | UACUGACUUUG |
| HAO1_exon4 | − | TTTA | 166 | TTTCTCTACTTGAATTC | 621 | UUUCUCUACUUGAAUUCAU |
| | | | | ATACTGACTTTGT | | ACUGACUUUGU |
| HAO1_exon4 | − | ATTT | 167 | CTCTACTTGAATTCATA | 622 | CUCUACUUGAAUUCAUACU |
| | | | | CTGACTTTGTGAT | | GACUUUGUGAU |
| HAO1_exon4 | − | CTTG | 168 | CTGCATATGTGGCTAAA | 623 | CUGCAUAUGUGGCUAAAGC |
| | | | | GCAATAGACCCAT | | AAUAGACCCAU |
| HAO1_exon4 | − | ATTT | 169 | CTTATTTTAGGATGAAA | 624 | CUUAUUUUAGGAUGAAAAA |
| | | | | AATTTTGAAACCA | | UUUUGAAACCA |
| HAO1_exon4 | − | TTTG | 170 | GAGACGACAGTGGACTT | 625 | GAGACGACAGUGGACUUGC |
| | | | | GCTGCATATGTGG | | UGCAUAUGUGG |
| HAO1_exon4 | − | ATTT | 171 | TGGAGACGACAGTGGAC | 626 | UGGAGACGACAGUGGACUU |
| | | | | TTGCTGCATATGT | | GCUGCAUAUGU |
| HAO1_exon4 | − | TTTC | 172 | TCCTGAGGAAAATTTTG | 627 | UCCUGAGGAAAAUUUUGGA |
| | | | | GAGACGACAGTGG | | GACGACAGUGG |
| HAO1_exon4 | − | TTTT | 173 | CTCCTGAGGAAAATTTT | 628 | CUCCUGAGGAAAAUUUUGG |
| | | | | GGAGACGACAGTG | | AGACGACAGUG |
| HAO1_exon4 | − | ATTT | 174 | TCTCCTGAGGAAAATTT | 629 | UCUCCUGAGGAAAAUUUUG |
| | | | | TGGAGACGACAGT | | GAGACGACAGU |
| HAO1_exon4 | − | TTTA | 175 | TCATTTTCTCCTGAGGA | 630 | UCAUUUUCUCCUGAGGAAA |
| | | | | AAATTTTGGAGAC | | AUUUUGGAGAC |
| HAO1_exon4 | − | CTTT | 176 | ATCATTTTCTCCTGAGG | 631 | AUCAUUUUCUCCUGAGGAA |
| | | | | AAAATTTTGGAGA | | AAUUUUGGAGA |
| HAO1_exon4 | − | TTTG | 177 | AAACCAGTACTTTATCA | 632 | AAACCAGUACUUUAUCAUU |
| | | | | TTTTCTCCTGAGG | | UUCUCCUGAGG |
| HAO1_exon4 | − | TTTT | 178 | GAAACCAGTACTTTATC | 633 | GAAACCAGUACUUUAUCAU |
| | | | | ATTTTCTCCTGAG | | UUUCUCCUGAG |
| HAO1_exon4 | − | ATTT | 179 | TGAAACCAGTACTTTAT | 634 | UGAAACCAGUACUUUAUCA |
| | | | | CATTTTCTCCTGA | | UUUUCUCCUGA |
| HAO1_exon4 | − | TTTT | 180 | AGGATGAAAAATTTTGA | 635 | AGGAUGAAAAAUUUUGAAA |
| | | | | AACCAGTACTTTA | | CCAGUACUUUA |
| HAO1_exon4 | − | ATTT | 181 | TAGGATGAAAAATTTTG | 636 | UAGGAUGAAAAAUUUUGAA |
| | | | | AAACCAGTACTTT | | ACCAGUACUUU |
| HAO1_exon4 | − | CTTA | 182 | TTTTAGGATGAAAAATT | 637 | UUUUAGGAUGAAAAAUUUU |
| | | | | TTGAAACCAGTAC | | GAAACCAGUAC |
| HAO1_exon4 | − | TTTT | 183 | GGAGACGACAGTGGACT | 638 | GGAGACGACAGUGGACUUG |
| | | | | TGCTGCATATGTG | | CUGCAUAUGUG |
| HAO1_exon4 | + | TTTG | 184 | CAACAATTGGCAATGAT | 639 | CAACAAUUGGCAAUGAUGU |
| | | | | GTCAGTCTTCTCA | | CAGUCUUCUCA |
| HAO1_exon4 | − | CTTG | 185 | AATTCATACTGACTTTG | 640 | AAUUCAUACUGACUUUGUG |
| | | | | TGATCCTTTGTG | | AUCCUUUGUG |
| HAO1_exon4 | − | ATTC | 186 | ATACTGACTTTGTGATC | 641 | AUACUGACUUUGUGAUCCU |
| | | | | CTTTGTG | | UUGUG |
| HAO1_exon5 | − | GTTA | 187 | AGTTACAGTTTCCCTAA | 642 | AGUUACAGUUUCCCUAAGG |
| | | | | GGTGCTTGTTTTA | | UGCUUGUUUUA |
| HAO1_exon5 | + | ATTC | 188 | AAGCCATGTTTAACAGC | 643 | AAGCCAUGUUUAACAGCCU |
| | | | | CTCCCTGGCATCA | | CCCUGGCAUCA |
| HAO1_exon5 | + | TTTA | 189 | ACAGCCTCCCTGGCATC | 644 | ACAGCCUCCCUGGCAUCAU |
| | | | | ATCACCTGGAGAG | | CACCUGGAGAG |
| HAO1_exon5 | + | GTTT | 190 | AACAGCCTCCCTGGCAT | 645 | AACAGCCUCCCUGGCAUCA |
| | | | | CATCACCTGGAGA | | UCACCUGGAGA |
| HAO1_exon5 | + | ATTC | 191 | GACACCAAGATCCCATT | 646 | GACACCAAGAUCCCAUUCA |
| | | | | CAAGCCATGTTTA | | AGCCAUGUUUA |
| HAO1_exon5 | + | GTTG | 192 | TCGAGCCCCATGATTCG | 647 | UCGAGCCCCAUGAUUCGAC |
| | | | | ACACCAAGATCCC | | ACCAAGAUCCC |
| HAO1_exon5 | + | CTTA | 193 | GCGTCTGCCAAAACTCA | 648 | GCGUCUGCCAAAACUCACA |
| | | | | CAGTGGCTGGCAC | | GUGGCUGGCAC |
| HAO1_exon5 | − | TTTG | 194 | GCAGACGCTAAGATTTC | 649 | GCAGACGCUAAGAUUUCCU |
| | | | | CTTTTGGAGTTCC | | UUUGGAGUUCC |
| HAO1_exon5 | − | GTTT | 195 | TGGCAGACGCTAAGATT | 650 | UGGCAGACGCUAAGAUUUC |
| | | | | TCCTTTTGGAGTT | | CUUUUGGAGUU |
| HAO1_exon5 | − | CTTG | 196 | GTGTCGAATCATGGGGC | 651 | GUGUCGAAUCAUGGGGCUC |
| | | | | TCGACAACTCGAT | | GACAACUCGAU |
| HAO1_exon5 | − | TTTT | 197 | GGCAGACGCTAAGATTT | 652 | GGCAGACGCUAAGAUUUCC |
| | | | | CCTTTTGGAGTTC | | UUUUGGAGUUC |
| HAO1_exon5 | − | GTTA | 198 | AACATGGCTTGAATGGG | 653 | AACAUGGCUUGAAUGGGAU |
| | | | | ATCTTGGTGTCGA | | CUUGGUGUCGA |
| HAO1_exon5 | − | TTTA | 199 | CTCTCTCCAGGTGATGA | 654 | CUCUCUCCAGGUGAUGAUG |
| | | | | TGCCAGGGAGGCT | | CCAGGGAGGCU |
| HAO1_exon5 | − | TTTT | 200 | ACTCTCTCCAGGTGATG | 655 | ACUCUCUCCAGGUGAUGAU |
| | | | | ATGCCAGGGAGGC | | GCCAGGGAGGC |
| HAO1_exon5 | − | GTTT | 201 | TACTCTCTCCAGGTGAT | 656 | UACUCUCUCCAGGUGAUGA |
| | | | | GATGCCAGGGAGG | | UGCCAGGGAGG |
| HAO1_exon5 | − | CTTG | 202 | TTTTACTCTCTCCAGGT | 657 | UUUUACUCUCUCCAGGUGA |
| | | | | GATGATGCCAGGG | | UGAUGCCAGGG |
| HAO1_exon5 | − | TTTC | 203 | CCTAAGGTGCTTGTTTT | 658 | CCUAAGGUGCUUGUUUUAC |
| | | | | ACTCTCTCCAGGT | | UCUCUCCAGGU |
| HAO1_exon5 | − | GTTT | 204 | CCCTAAGGTGCTTGTTT | 659 | CCCUAAGGUGCUUGUUUUA |
| | | | | TACTCTCTCCAGG | | CUCUCUCCAGG |
| HAO1_exon5 | − | GTTA | 205 | CAGTTTCCCTAAGGTGC | 660 | CAGUUUCCCUAAGGUGCUU |
| | | | | TTGTTTTACTCTC | | GUUUUACUCUC |
| HAO1_exon5 | − | CTTG | 206 | AATGGGATCTTGGTGTC | 661 | AAUGGGAUCUUGGUGUCGA |
| | | | | GAATCATGGGGCT | | AUCAUGGGGCU |
| HAO1_exon5 | − | ATTT | 207 | CCTTTTGGAGTTCCCAT | 662 | CCUUUUGGAGUUCCCAUUU |
| | | | | TTCCATC | | CCAUC |
| HAO1_exon5 | − | TTTC | 208 | CTTTTGGAGTTCCCATT | 663 | CUUUUGGAGUUCCCAUUUC |
| | | | | TCCATC | | CAUC |
| HAO1_exon5 | + | CTTA | 209 | GGGAAACTGTAACTTAA | 664 | GGGAAACUGUAACUUAACA |
| | | | | CAGGCAG | | GGCAG |
| HAO1_exon6 | − | TTTA | 210 | CAACTTTCTTTTCTTTT | 665 | CAACUUUCUUUUCUUUUAU |
| | | | | ATGATCTTTAAGT | | GAUCUUUAAGU |
| HAO1_exon6 | − | ATTC | 211 | CGGTTGGCCATGGCTCT | 666 | CGGUUGGCCAUGGCUCUGA |
| | | | | GAGTGGTAAGACT | | GUGGUAAGACU |
| HAO1_exon6 | − | GTTG | 212 | GCCATGGCTCTGAGTGG | 667 | GCCAUGGCUCUGAGUGGUA |
| | | | | TAAGACTCATTCT | | AGACUCAUUCU |
| HAO1_exon6 | − | ATTC | 213 | TTGTTTACAACTTTCTT | 668 | UUGUUUACAACUUUCUUUU |
| | | | | TTCTTTTATGATC | | CUUUUAUGAUC |
| HAO1_exon6 | − | CTTG | 214 | TTTACAACTTTCTTTTC | 669 | UUUACAACUUUCUUUUCUU |
| | | | | TTTTATGATCTTT | | UUAUGAUCUUU |
| HAO1_exon6 | − | GTTT | 215 | ACAACTTTCTTTTCTTT | 670 | ACAACUUUCUUUUCUUUUA |
| | | | | TATGATCTTTAAG | | UGAUCUUUAAG |
| HAO1_exon6 | + | CTTA | 216 | AAGATCATAAAAGAAAA | 671 | AAGAUCAUAAAAGAAAAGA |
| | | | | GAAAGTTGTAAAC | | AAGUUGUAAAC |
| HAO1_exon6 | + | GTTG | 217 | TCTATTTTATATATTCA | 672 | UCUAUUUUAUAUAUUCAUU |
| | | | | TTTCTTTGTCCAG | | UCUUUGUCCAG |
| HAO1_exon6 | + | CTTA | 218 | CCACTCAGAGCCATGGC | 673 | CCACUCAGAGCCAUGGCCA |
| | | | | CAACCGGAATTCT | | ACCGGAAUUCU |
| HAO1_exon6 | + | ATTC | 219 | TTCCTTTAGTATCTCGA | 674 | UUCCUUUAGUAUCUCGAGG |
| | | | | GGACATCTTGAAC | | ACAUCUUGAAC |
| HAO1_exon6 | + | CTTC | 220 | CTTTAGTATCTCGAGGA | 675 | CUUUAGUAUCUCGAGGACA |
| | | | | CATCTTGAACACC | | UCUUGAACACC |
| HAO1_exon6 | + | CTTT | 221 | AGTATCTCGAGGACATC | 676 | AGUAUCUCGAGGACAUCUU |
| | | | | TTGAACACCTTTC | | GAACACCUUUC |
| HAO1_exon6 | + | TTTA | 222 | GTATCTCGAGGACATCT | 677 | GUAUCUCGAGGACAUCUUG |
| | | | | TGAACACCTTTCT | | AACACCUUUCU |
| HAO1_exon6 | − | GTTC | 223 | AAGATGTCCTCGAGATA | 678 | AAGAUGUCCUCGAGAUACU |
| | | | | CTAAAGGAAGAAT | | AAAGGAAGAAU |
| HAO1_exon6 | + | GTTG | 224 | TAAACAAGAATGAGTCT | 679 | UAAACAAGAAUGAGUCUUA |
| | | | | TACCACTCAGAGC | | CCACUCAGAGC |
| HAO1_exon6 | − | GTTA | 225 | GGGGGAGAAAGGTGTTC | 680 | GGGGGAGAAAGGUGUUCAA |
| | | | | AAGATGTCCTCGA | | GAUGUCCUCGA |
| HAO1_exon6 | − | CTTT | 226 | CCAGGTAACTGGACAAA | 681 | CCAGGUAACUGGACAAAGA |
| | | | | GAAATGAATATAT | | AAUGAAUAUAU |
| HAO1_exon6 | − | TTTC | 227 | ACTTGGTTAGGGGGAGA | 682 | ACUUGGUUAGGGGGAGAAA |
| | | | | AAGGTGTTCAAGA | | GGUGUUCAAGA |
| HAO1_exon6 | − | TTTT | 228 | CACTTGGTTAGGGGGAG | 683 | CACUUGGUUAGGGGGAGAA |
| | | | | AAAGGTGTTCAAG | | AGGUGUUCAAG |
| HAO1_exon6 | − | CTTT | 229 | TCACTTGGTTAGGGGGA | 684 | UCACUUGGUUAGGGGGAGA |
| | | | | GAAAGGTGTTCAA | | AAGGUGUUCAA |
| HAO1_exon6 | − | GTTC | 230 | TGAATCACTCTGTATCT | 685 | UGAAUCACUCUGUAUCUUU |
| | | | | TTTCACTTGGTTA | | UCACUUGGUUA |
| HAO1_exon6 | − | TTTA | 231 | GTTCTGAATCACTCTGT | 686 | GUUCUGAAUCACUCUGUAU |
| | | | | ATCTTTTCACTTG | | CUUUUCACUUG |
| HAO1_exon6 | − | ATTT | 232 | AGTTCTGAATCACTCTG | 687 | AGUUCUGAAUCACUCUGUA |
| | | | | TATCTTTTCACTT | | UCUUUUCACUU |
| HAO1_exon6 | − | CTTG | 233 | ACAGTAAAACAAATGAA | 688 | ACAGUAAAACAAAUGAAUA |
| | | | | TAAAACAAGTCAG | | AAACAAGUCAG |
| HAO1_exon6 | − | TTTC | 234 | CAGGTAACTGGACAAAG | 689 | CAGGUAACUGGACAAAGAA |
| | | | | AAATGAATATATA | | AUGAAUAUAUA |
| HAO1_exon6 | + | CTTG | 235 | AACACCTTTCTCCCCCT | 690 | AACACCUUUCUCCCCCUAA |
| | | | | AACCAAGTGAAAA | | CCAAGUGAAAA |
| HAO1_exon6 | − | CTTA | 236 | GCTTTCCAGGTAACTGG | 691 | GCUUUCCAGGUAACUGGAC |
| | | | | ACAAAGAAATGAA | | AAAGAAAUGAA |
| HAO1_exon6 | − | TTTG | 237 | GGGCTTAGCTTTCCAGG | 692 | GGGCUUAGCUUUCCAGGUA |
| | | | | TAACTGGACAAAG | | ACUGGACAAAG |
| HAO1_exon6 | − | GTTT | 238 | GGGGCTTAGCTTTCCAG | 693 | GGGGCUUAGCUUUCCAGGU |
| | | | | GTAACTGGACAAA | | AACUGGACAAA |
| HAO1_exon6 | − | TTTG | 239 | TGGGGAGACCAATCGTT | 694 | UGGGGAGACCAAUCGUUUG |
| | | | | TGGGGCTTAGCTT | | GGGCUUAGCUU |
| HAO1_exon6 | − | GTTT | 240 | GTGGGGAGACCAATCGT | 695 | GUGGGGAGACCAAUCGUUU |
| | | | | TTGGGGCTTAGCT | | GGGGCUUAGCU |
| HAO1_exon6 | − | CTTG | 241 | GTTAGGGGGAGAAAGGT | 696 | GUUAGGGGGAGAAAGGUGU |
| | | | | GTTCAAGATGTCC | | UCAAGAUGUCC |
| HAO1_exon6 | + | CTTT | 242 | CTCCCCCTAACCAAGTG | 697 | CUCCCCCUAACCAAGUGAA |
| | | | | AAAAGATACAGAG | | AAGAUACAGAG |
| HAO1_exon6 | + | GTTT | 243 | TACTGTCAAGTTGTCTA | 698 | UACUGUCAAGUUGUCUAUU |
| | | | | TTTTATATATTCA | | UUAUAUAUUCA |
| HAO1_exon6 | + | ATTC | 244 | AGAACTAAATCAGTCTG | 699 | AGAACUAAAUCAGUCUGAC |
| | | | | ACTTGTTTTATTC | | UUGUUUUAUUC |
| HAO1_exon6 | + | GTTC | 245 | AATAATGTGACTCTATT | 700 | AAUAAUGUGACUCUAUUAA |
| | | | | AACACTGAATTGT | | CACUGAAUUGU |
| HAO1_exon6 | + | TTTC | 246 | TGGCAGAACATCAATCT | 701 | UGGCAGAACAUCAAUCUGG |
| | | | | GGGGAAAGAAAAG | | GGAAAGAAAAG |
| HAO1_exon6 | + | ATTT | 247 | CTGGCAGAACATCAATC | 702 | CUGGCAGAACAUCAAUCUG |
| | | | | TGGGGAAAGAAAA | | GGGAAAGAAAA |
| HAO1_exon6 | + | CTTC | 248 | CACAGCCTCCACAATTT | 703 | CACAGCCUCCACAAUUUCU |
| | | | | CTGGCAGAACATC | | GGCAGAACAUC |
| HAO1_exon6 | + | CTTC | 249 | CCTTCCACAGCCTCCAC | 704 | CCUUCCACAGCCUCCACAA |
| | | | | AATTTCTGGCAGA | | UUUCUGGCAGA |
| HAO1_exon6 | + | CTTC | 250 | CACCTTCCCTTCCACAG | 705 | CACCUUCCCUUCCACAGCC |
| | | | | CCTCCACAATTTC | | UCCACAAUUUC |
| HAO1_exon6 | + | TTTC | 251 | CGCACACCCCCGTCCAG | 706 | CGCACACCCCCGUCCAGGA |
| | | | | GAAGACTTCCACC | | AGACUUCCACC |
| HAO1_exon6 | + | CTTT | 252 | CCGCACACCCCCGTCCA | 707 | CCGCACACCCCCGUCCAGG |
| | | | | GGAAGACTTCCAC | | AAGACUUCCAC |
| HAO1_exon6 | + | TTTC | 253 | AGAACATCAGTGCCTTT | 708 | AGAACAUCAGUGCCUUUCC |
| | | | | CCGCACACCCCCG | | GCACACCCCCG |
| HAO1_exon6 | + | CTTT | 254 | CAGAACATCAGTGCCTT | 709 | CAGAACAUCAGUGCCUUUC |
| | | | | TCCGCACACCCCC | | CGCACACCCCC |
| HAO1_exon6 | + | CTTG | 255 | GCGCCAAGAGCCAGAGC | 710 | GCGCCAAGAGCCAGAGCUU |
| | | | | TTTCAGAACATCA | | UCAGAACAUCA |
| HAO1_exon6 | + | ATTG | 256 | GTCTCCCCACAAACACA | 711 | GUCUCCCCACAAACACAGC |
| | | | | GCCTTGGCGCCAA | | CUUGGCGCCAA |
| HAO1_exon6 | + | GTTA | 257 | CCTGGAAAGCTAAGCCC | 712 | CCUGGAAAGCUAAGCCCCA |
| | | | | CAAACGATTGGTC | | AACGAUUGGUC |
| HAO1_exon6 | + | TTTG | 258 | TCCAGTTACCTGGAAAG | 713 | UCCAGUUACCUGGAAAGCU |
| | | | | CTAAGCCCCAAAC | | AAGCCCCAAAC |
| HAO1_exon6 | + | CTTT | 259 | GTCCAGTTACCTGGAAA | 714 | GUCCAGUUACCUGGAAAGC |
| | | | | GCTAAGCCCCAAA | | UAAGCCCCAAA |
| HAO1_exon6 | + | TTTC | 260 | TTTGTCCAGTTACCTGG | 715 | UUUGUCCAGUUACCUGGAA |
| | | | | AAAGCTAAGCCCC | | AGCUAAGCCCC |
| HAO1_exon6 | + | ATTT | 261 | CTTTGTCCAGTTACCTG | 716 | CUUUGUCCAGUUACCUGGA |
| | | | | GAAAGCTAAGCCC | | AAGCUAAGCCC |
| HAO1_exon6 | + | CTTG | 262 | TTTTATTCATTTGTTTT | 717 | UUUUAUUCAUUUGUUUUAC |
| | | | | ACTGTCAAGTTGT | | UGUCAAGUUGU |
| HAO1_exon6 | + | GTTT | 263 | TATTCATTTGTTTTACT | 718 | UAUUCAUUUGUUUUACUGU |
| | | | | GTCAAGTTGTCTA | | CAAGUUGUCUA |
| HAO1_exon6 | + | TTTT | 264 | ATTCATTTGTTTTACTG | 719 | AUUCAUUUGUUUUACUGUC |
| | | | | TCAAGTTGTCTAT | | AAGUUGUCUAU |
| HAO1_exon6 | + | TTTA | 265 | TTCATTTGTTTTACTGT | 720 | UUCAUUUGUUUUACUGUCA |
| | | | | CAAGTTGTCTATT | | AGUUGUCUAUU |
| HAO1_exon6 | + | ATTC | 266 | ATTTGTTTTACTGTCAA | 721 | AUUUGUUUUACUGUCAAGU |
| | | | | GTTGTCTATTTTA | | UGUCUAUUUUA |
| HAO1_exon6 | + | ATTT | 267 | GTTTTACTGTCAAGTTG | 722 | GUUUUACUGUCAAGUUGUC |
| | | | | TCTATTTTATATA | | UAUUUUAUAUA |
| HAO1_exon6 | + | TTTC | 268 | TCCCCCTAACCAAGTGA | 723 | UCCCCCUAACCAAGUGAAA |
| | | | | AAAGATACAGAGT | | AGAUACAGAGU |
| HAO1_exon6 | + | TTTG | 269 | TTTTACTGTCAAGTTGT | 724 | UUUUACUGUCAAGUUGUCU |
| | | | | CTATTTTATATAT | | AUUUUAUAUAU |
| HAO1_exon6 | + | TTTT | 270 | ACTGTCAAGTTGTCTAT | 725 | ACUGUCAAGUUGUCUAUUU |
| | | | | TTTATATATTCAT | | UAUAUAUUCAU |
| HAO1_exon6 | + | TTTA | 271 | CTGTCAAGTTGTCTATT | 726 | CUGUCAAGUUGUCUAUUUU |
| | | | | TTATATATTCATT | | AUAUAUUCAUU |
| HAO1_exon6 | + | ATTT | 272 | TATATATTCATTTCTTT | 727 | UAUAUAUUCAUUUCUUUGU |
| | | | | GTCCAGTTACCTG | | CCAGUUACCUG |
| HAO1_exon6 | + | TTTT | 273 | ATATATTCATTTCTTTG | 728 | AUAUAUUCAUUUCUUUGUC |
| | | | | TCCAGTTACCTGG | | CAGUUACCUGG |
| HAO1_exon6 | + | TTTA | 274 | TATATTCATTTCTTTGT | 729 | UAUAUUCAUUUCUUUGUCC |
| | | | | CCAGTTACCTGGA | | AGUUACCUGGA |
| HAO1_exon6 | + | ATTC | 275 | ATTTCTTTGTCCAGTTA | 730 | AUUUCUUUGUCCAGUUACC |
| | | | | CCTGGAAAGCTAA | | UGGAAAGCUAA |
| HAO1_exon6 | − | CTTG | 276 | GCGCCAAGGCTGTGTTT | 731 | GCGCCAAGGCUGUGUUUGU |
| | | | | GTGGGGAGACCAA | | GGGGAGACCAA |
| HAO1_exon6 | − | GTTC | 277 | TGAAAGCTCTGGCTCTT | 732 | UGAAAGCUCUGGCUCUUGG |
| | | | | GGCGCCAAGGCTG | | CGCCAAGGCUG |
| HAO1_exon6 | − | ATTG | 278 | TGGAGGCTGTGGAAGGG | 733 | UGGAGGCUGUGGAAGGGAA |
| | | | | AAGGTGGAAGTCT | | GGUGGAAGUCU |
| HAO1_exon6 | − | ATTA | 279 | TTGAACTTTTCTTTCCC | 734 | UUGAACUUUUCUUUCCCCA |
| | | | | CAGATTGATGTTC | | GAUUGAUGUUC |
| HAO1_exon6 | − | GTTC | 280 | TGCCAGAAATTGTGGAG | 735 | UGCCAGAAAUUGUGGAGGC |
| | | | | GCTGTGGAAGGGA | | UGUGGAAGGGA |
| HAO1_exon6 | − | ATTG | 281 | ATGTTCTGCCAGAAATT | 736 | AUGUUCUGCCAGAAAUUGU |
| | | | | GTGGAGGCTGTGG | | GGAGGCUGUGG |
| HAO1_exon6 | − | TTTC | 282 | CCCAGATTGATGTTCTG | 737 | CCCAGAUUGAUGUUCUGCC |
| | | | | CCAGAAATTGTGG | | AGAAAUUGUGG |
| HAO1_exon6 | − | CTTT | 283 | CCCCAGATTGATGTTCT | 738 | CCCCAGAUUGAUGUUCUGC |
| | | | | GCCAGAAATTGTG | | CAGAAAUUGUG |
| HAO1_exon6 | − | TTTC | 284 | TTTCCCCAGATTGATGT | 739 | UUUCCCCAGAUUGAUGUUC |
| | | | | TCTGCCAGAAATT | | UGCCAGAAAUU |
| HAO1_exon6 | − | TTTT | 285 | CTTTCCCCAGATTGATG | 740 | CUUUCCCCAGAUUGAUGUU |
| | | | | TTCTGCCAGAAAT | | CUGCCAGAAAU |
| HAO1_exon6 | − | CTTT | 286 | TCTTTCCCCAGATTGAT | 741 | UCUUUCCCCAGAUUGAUGU |
| | | | | GTTCTGCCAGAAA | | UCUGCCAGAAA |
| HAO1_exon6 | − | ATTG | 287 | AACTTTTCTTTCCCCAG | 742 | AACUUUUCUUUCCCCAGAU |
| | | | | ATTGATGTTCTGC | | UGAUGUUCUGC |
| HAO1_exon6 | − | GTTA | 288 | ATAGAGTCACATTATTG | 743 | AUAGAGUCACAUUAUUGAA |
| | | | | AACTTTTCTTTCC | | CUUUUCUUUCC |
| HAO1_exon6 | − | ATTC | 289 | AGTGTTAATAGAGTCAC | 744 | AGUGUUAAUAGAGUCACAU |
| | | | | ATTATTGAACTTT | | UAUUGAACUUU |
| HAO1_exon6 | − | CTTC | 290 | CTGGACGGGGGTGTGCG | 745 | CUGGACGGGGGUGUGCGGA |
| | | | | GAAAGGCACTGAT | | AAGGCACUGAU |
| HAO1_exon6 | − | CTTT | 291 | CTTTTCTTTTATGATCT | 746 | CUUUUCUUUUAUGAUCUUU |
| | | | | TTAAGT | | AAGU |
| HAO1_exon6 | − | TTTC | 292 | TTTTCTTTTATGATCTT | 747 | UUUUUUUUAUGAUCUUUA |
| | | | | TAAGT | | AGU |
| HAO1_exon7 | − | ATTT | 293 | TTTCAGGGTGCCAGAAT | 748 | UUUCAGGGUGCCAGAAUGU |
| | | | | GTGAAAGTCATCG | | GAAAGUCAUCG |
| HAO1_exon7 | − | ATTA | 294 | TTTTTTCAGGGTGCCAG | 749 | UUUUUUCAGGGUGCCAGAA |
| | | | | AATGTGAAAGTCA | | UGUGAAAGUCA |
| HAO1_exon7 | − | ATTG | 295 | TAAGCTCAGGTTCAAAG | 750 | UAAGCUCAGGUUCAAAGUG |
| | | | | TGTTGGTAATGCC | | UUGGUAAUGCC |
| HAO1_exon7 | − | GTTC | 296 | ATATTAAATGTATGCAT | 751 | AUAUUAAAUGUAUGCAUUA |
| | | | | TATTTTTTCAGGG | | UUUUUUCAGGG |
| HAO1_exon7 | − | ATTC | 297 | AGTTCATATTAAATGTA | 752 | AGUUCAUAUUAAAUGUAUG |
| | | | | TGCATTATTTTTT | | CAUUAUUUUUU |
| HAO1_exon7 | − | TTTT | 298 | TTCAGGGTGCCAGAATG | 753 | UUCAGGGUGCCAGAAUGUG |
| | | | | TGAAAGTCATCGA | | AAAGUCAUCGA |
| HAO1_exon7 | − | ATTA | 299 | AATGTATGCATTATTTT | 754 | AAUGUAUGCAUUAUUUUUU |
| | | | | TTCAGGGTGCCAG | | CAGGGUGCCAG |
| HAO1_exon7 | − | TTTT | 300 | TCAGGGTGCCAGAATGT | 755 | UCAGGGUGCCAGAAUGUGA |
| | | | | GAAAGTCATCGAC | | AAGUCAUCGAC |
| HAO1_exon7 | − | TTTG | 301 | GCCGTTTCCAAGATCTG | 756 | GCCGUUUCCAAGAUCUGAC |
| | | | | ACAGTGCACAATA | | AGUGCACAAUA |
| HAO1_exon7 | − | TTTC | 302 | AGGGTGCCAGAATGTGA | 757 | AGGGUGCCAGAAUGUGAAA |
| | | | | AAGTCATCGACAA | | GUCAUCGACAA |
| HAO1_exon7 | − | ATTG | 303 | GTGAGGAAAAATCCTTT | 758 | GUGAGGAAAAAUCCUUUGG |
| | | | | GGCCGTTTCCAAG | | CCGUUUCCAAG |
| HAO1_exon7 | − | CTTT | 304 | GGCCGTTTCCAAGATCT | 759 | GGCCGUUUCCAAGAUCUGA |
| | | | | GACAGTGCACAAT | | CAGUGCACAAU |
| HAO1_exon7 | − | ATTG | 305 | CATTCAGTTCATATTAA | 760 | CAUUCAGUUCAUAUUAAAU |
| | | | | ATGTATGCATTAT | | GUAUGCAUUAU |
| HAO1_exon7 | − | GTTT | 306 | CCAAGATCTGACAGTGC | 761 | CCAAGAUCUGACAGUGCAC |
| | | | | ACAATATTTTCCC | | AAUAUUUUCCC |
| HAO1_exon7 | − | TTTC | 307 | CAAGATCTGACAGTGCA | 762 | CAAGAUCUGACAGUGCACA |
| | | | | CAATATTTTCCCA | | AUAUUUUCCCA |
| HAO1_exon7 | − | TTTT | 308 | CAGGGTGCCAGAATGTG | 763 | CAGGGUGCCAGAAUGUGAA |
| | | | | AAAGTCATCGACA | | AGUCAUCGACA |
| HAO1_exon7 | − | ATTA | 309 | TTGCATTCAGTTCATAT | 764 | UUGCAUUCAGUUCAUAUUA |
| | | | | TAAATGTATGCAT | | AAUGUAUGCAU |
| HAO1_exon7 | − | ATTG | 310 | GAGGTAGCAAACACTAA | 765 | GAGGUAGCAAACACUAAGG |
| | | | | GGTGAAAAGATAA | | UGAAAAGAUAA |
| HAO1_exon7 | − | GTTT | 311 | AGACAACGTCATCCCCT | 766 | AGACAACGUCAUCCCCUGG |
| | | | | GGCAGGCTAAAGT | | CAGGCUAAAGU |
| HAO1_exon7 | − | CTTA | 312 | AATTGTAAGCTCAGGTT | 767 | AAUUGUAAGCUCAGGUUCA |
| | | | | CAAAGTGTTGGTA | | AAGUGUUGGUA |
| HAO1_exon7 | − | GTTC | 313 | TTAAATTGTAAGCTCAG | 768 | UUAAAUUGUAAGCUCAGGU |
| | | | | GTTCAAAGTGTTG | | UCAAAGUGUUG |
| HAO1_exon7 | − | TTTA | 314 | AAACAGTGGTTCTTAAA | 769 | AAACAGUGGUUCUUAAAUU |
| | | | | TTGTAAGCTCAGG | | GUAAGCUCAGG |
| HAO1_exon7 | − | CTTT | 315 | AAAACAGTGGTTCTTAA | 770 | AAAACAGUGGUUCUUAAAU |
| | | | | ATTGTAAGCTCAG | | UGUAAGCUCAG |
| HAO1_exon7 | − | TTTA | 316 | CATGTCTTTAAAACAGT | 771 | CAUGUCUUUAAAACAGUGG |
| | | | | GGTTCTTAAATTG | | UUCUUAAAUUG |
| HAO1_exon7 | − | GTTT | 317 | ACATGTCTTTAAAACAG | 772 | ACAUGUCUUUAAAACAGUG |
| | | | | TGGTTCTTAAATT | | GUUCUUAAAUU |
| HAO1_exon7 | − | ATTC | 318 | TGTTTACATGTCTTTAA | 773 | UGUUUACAUGUCUUUAAAA |
| | | | | AACAGTGGTTCTT | | CAGUGGUUCUU |
| HAO1_exon7 | − | ATTA | 319 | ACCTGTATTCTGTTTAC | 774 | ACCUGUAUUCUGUUUACAU |
| | | | | ATGTCTTTAAAAC | | GUCUUUAAAAC |
| HAO1_exon7 | − | TTTA | 320 | TTAACCTGTATTCTGTT | 775 | UUAACCUGUAUUCUGUUUA |
| | | | | TACATGTCTTTAA | | CAUGUCUUUAA |
| HAO1_exon7 | − | GTTT | 321 | ATTAACCTGTATTCTGT | 776 | AUUAACCUGUAUUCUGUUU |
| | | | | TTACATGTCTTTA | | ACAUGUCUUUA |
| HAO1_exon7 | − | ATTG | 322 | TTTATTAACCTGTATTC | 777 | UUUAUUAACCUGUAUUCUG |
| | | | | TGTTTACATGTCT | | UUUACAUGUCU |
| HAO1_exon7 | − | ATTT | 323 | TCCCATCTGTATTATTT | 778 | UCCCAUCUGUAUUAUUUUU |
| | | | | TTTTTCAGCATGT | | UUUCAGCAUGU |
| HAO1_exon7 | − | TTTA | 324 | GTAAAATTGGAGGTAGC | 779 | GUAAAAUUGGAGGUAGCAA |
| | | | | AAACACTAAGGTG | | ACACUAAGGUG |
| HAO1_exon7 | − | CTTT | 325 | AGTAAAATTGGAGGTAG | 780 | AGUAAAAUUGGAGGUAGCA |
| | | | | CAAACACTAAGGT | | AACACUAAGGU |
| HAO1_exon7 | − | TTTA | 326 | GACAACGTCATCCCCTG | 781 | GACAACGUCAUCCCCUGGC |
| | | | | GCAGGCTAAAGTG | | AGGCUAAAGUG |
| HAO1_exon7 | − | ATTA | 327 | TTATTGCATTCAGTTCA | 782 | UUAUUGCAUUCAGUUCAUA |
| | | | | TATTAAATGTATG | | UUAAAUGUAUG |
| HAO1_exon7 | − | TTTT | 328 | CCCATCTGTATTATTTT | 783 | CCCAUCUGUAUUAUUUUUU |
| | | | | TTTTCAGCATGTA | | UUCAGCAUGUA |
| HAO1_exon7 | − | TTTT | 329 | TTCAGCATGTATTACTT | 784 | UUCAGCAUGUAUUACUUGA |
| | | | | GACAAAGAGACAC | | CAAAGAGACAC |
| HAO1_exon7 | − | ATTA | 330 | TTTTTTTTCAGCATGTA | 785 | UUUUUUUUCAGCAUGUAUU |
| | | | | TTACTTGACAAAG | | ACUUGACAAAG |
| HAO1_exon7 | − | TTTC | 331 | ATTGCTTTTGACTTTTC | 786 | AUUGCUUUUGACUUUUCAA |
| | | | | AATGGGTGTCCTA | | UGGGUGUCCUA |
| HAO1_exon7 | − | ATTG | 332 | CTTTTGACTTTTCAATG | 787 | CUUUUGACUUUUCAAUGGG |
| | | | | GGTGTCCTAGGAA | | UGUCCUAGGAA |
| HAO1_exon7 | − | CTTT | 333 | TGACTTTTCAATGGGTG | 788 | UGACUUUUCAAUGGGUGUC |
| | | | | TCCTAGGAACCTT | | CUAGGAACCUU |
| HAO1_exon7 | − | TTTT | 334 | GACTTTTCAATGGGTGT | 789 | GACUUUUCAAUGGGUGUCC |
| | | | | CCTAGGAACCTTT | | UAGGAACCUUU |
| HAO1_exon7 | − | TTTG | 335 | ACTTTTCAATGGGTGTC | 790 | ACUUUUCAAUGGGUGUCCU |
| | | | | CTAGGAACCTTTT | | AGGAACCUUUU |
| HAO1_exon7 | − | CTTT | 336 | TCAATGGGTGTCCTAGG | 791 | UCAAUGGGUGUCCUAGGAA |
| | | | | AACCTTTTAGAAA | | CCUUUUAGAAA |
| HAO1_exon7 | − | TTTT | 337 | CAATGGGTGTCCTAGGA | 792 | CAAUGGGUGUCCUAGGAAC |
| | | | | ACCTTTTAGAAAG | | CUUUUAGAAAG |
| HAO1_exon7 | − | TTTC | 338 | AATGGGTGTCCTAGGAA | 793 | AAUGGGUGUCCUAGGAACC |
| | | | | CCTTTTAGAAAGA | | UUUUAGAAAGA |
| HAO1_exon7 | − | CTTT | 339 | TAGAAAGAAATGGACTT | 794 | UAGAAAGAAAUGGACUUUC |
| | | | | TCATCCTGGAAAT | | AUCCUGGAAAU |
| HAO1_exon7 | − | TTTT | 340 | AGAAAGAAATGGACTTT | 795 | AGAAAGAAAUGGACUUUCA |
| | | | | CATCCTGGAAATA | | UCCUGGAAAUA |
| HAO1_exon7 | − | TTTA | 341 | GAAAGAAATGGACTTTC | 796 | GAAAGAAAUGGACUUUCAU |
| | | | | ATCCTGGAAATAT | | CCUGGAAAUAU |
| HAO1_exon7 | − | CTTT | 342 | CATCCTGGAAATATATT | 797 | CAUCCUGGAAAUAUAUUAA |
| | | | | AACTGTTAAAAAG | | CUGUUAAAAAG |
| HAO1_exon7 | − | TTTC | 343 | ATCCTGGAAATATATTA | 798 | AUCCUGGAAAUAUAUUAAC |
| | | | | ACTGTTAAAAAGA | | UGUUAAAAAGA |
| HAO1_exon7 | − | ATTA | 344 | ACTGTTAAAAAGAAAAC | 799 | ACUGUUAAAAAGAAAACAU |
| | | | | ATTGAAAATGTGT | | UGAAAAUGUGU |
| HAO1_exon7 | − | GTTA | 345 | AAAAGAAAACATTGAAA | 800 | AAAAGAAAACAUUGAAAAU |
| | | | | ATGTGTTTAGACA | | GUGUUUAGACA |
| HAO1_exon7 | − | ATTT | 346 | CATTGCTTTTGACTTTT | 801 | CAUUGCUUUUGACUUUUCA |
| | | | | CAATGGGTGTCCT | | AUGGGUGUCCU |
| HAO1_exon7 | − | TTTC | 347 | CCATCTGTATTATTTTT | 802 | CCAUCUGUAUUAUUUUUUU |
| | | | | TTTCAGCATGTAT | | UCAGCAUGUAU |
| HAO1_exon7 | − | TTTA | 348 | TTTCATTGCTTTTGACT | 803 | UUUCAUUGCUUUUGACUUU |
| | | | | TTTCAATGGGTGT | | UCAAUGGGUGU |
| HAO1_exon7 | − | CTTT | 349 | TATTTCATTGCTTTTGA | 804 | UAUUUCAUUGCUUUUGACU |
| | | | | CTTTTCAATGGGT | | UUUCAAUGGGU |
| HAO1_exon7 | − | ATTG | 350 | AAAATGTGTTTAGACAA | 805 | AAAAUGUGUUUAGACAACG |
| | | | | CGTCATCCCCTGG | | UCAUCCCCUGG |
| HAO1_exon7 | − | ATTT | 351 | TTTTTCAGCATGTATTA | 806 | UUUUUCAGCAUGUAUUACU |
| | | | | CTTGACAAAGAGA | | UGACAAAGAGA |
| HAO1_exon7 | − | TTTT | 352 | TTTTCAGCATGTATTAC | 807 | UUUUCAGCAUGUAUUACUU |
| | | | | TTGACAAAGAGAC | | GACAAAGAGAC |
| HAO1_exon7 | − | TTTT | 353 | TTTCAGCATGTATTACT | 808 | UUUCAGCAUGUAUUACUUG |
| | | | | TGACAAAGAGACA | | ACAAAGAGACA |
| HAO1_exon7 | − | TTTT | 354 | TCAGCATGTATTACTTG | 809 | UCAGCAUGUAUUACUUGAC |
| | | | | ACAAAGAGACACT | | AAAGAGACACU |
| HAO1_exon7 | − | TTTT | 355 | CAGCATGTATTACTTGA | 810 | CAGCAUGUAUUACUUGACA |
| | | | | CAAAGAGACACTG | | AAGAGACACUG |
| HAO1_exon7 | − | TTTC | 356 | AGCATGTATTACTTGAC | 811 | AGCAUGUAUUACUUGACAA |
| | | | | AAAGAGACACTGT | | AGAGACACUGU |
| HAO1_exon7 | − | ATTA | 357 | CTTGACAAAGAGACACT | 812 | CUUGACAAAGAGACACUGU |
| | | | | GTGCAGAGGGTGA | | GCAGAGGGUGA |
| HAO1_exon7 | − | CTTG | 358 | ACAAAGAGACACTGTGC | 813 | ACAAAGAGACACUGUGCAG |
| | | | | AGAGGGTGACCAC | | AGGGUGACCAC |
| HAO1_exon7 | − | ATTC | 359 | CCCACTTCAATACAAAG | 814 | CCCACUUCAAUACAAAGGG |
| | | | | GGTGTCGTTCTTT | | UGUCGUUCUUU |
| HAO1_exon7 | − | CTTC | 360 | AATACAAAGGGTGTCGT | 815 | AAUACAAAGGGUGUCGUUC |
| | | | | TCTTTTCCAACAA | | UUUUCCAACAA |
| HAO1_exon7 | − | GTTC | 361 | TTTTCCAACAAAATAGC | 816 | UUUUCCAACAAAAUAGCAA |
| | | | | AATCCCTTTTATT | | UCCCUUUUAUU |
| HAO1_exon7 | − | CTTT | 362 | TCCAACAAAATAGCAAT | 817 | UCCAACAAAAUAGCAAUCC |
| | | | | CCCTTTTATTTCA | | CUUUUAUUUCA |
| HAO1_exon7 | − | TTTT | 363 | CCAACAAAATAGCAATC | 818 | CCAACAAAAUAGCAAUCCC |
| | | | | CCTTTTATTTCAT | | UUUUAUUUCAU |
| HAO1_exon7 | − | TTTC | 364 | CAACAAAATAGCAATCC | 819 | CAACAAAAUAGCAAUCCCU |
| | | | | CTTTTATTTCATT | | UUUAUUUCAUU |
| HAO1_exon7 | − | TTTT | 365 | ATTTCATTGCTTTTGAC | 820 | AUUUCAUUGCUUUUGACUU |
| | | | | TTTTCAATGGGTG | | UUCAAUGGGUG |
| HAO1_exon7 | − | GTTC | 366 | AAAGTGTTGGTAATGCC | 821 | AAAGUGUUGGUAAUGCCUG |
| | | | | TGATTCACAACTT | | AUUCACAACUU |
| HAO1_exon7 | + | ATTT | 367 | CTCTCTAAGAAGTAACA | 822 | CUCUCUAAGAAGUAACAUA |
| | | | | TACATCCTAAAAC | | CAUCCUAAAAC |
| HAO1_exon7 | − | ATTC | 368 | ACAACTTTGAGAAGGTA | 823 | ACAACUUUGAGAAGGUAGC |
| | | | | GCACTGGAGAGAA | | ACUGGAGAGAA |
| HAO1_exon7 | + | TTTC | 369 | ACCTTAGTGTTTGCTAC | 824 | ACCUUAGUGUUUGCUACCU |
| | | | | CTCCAATTTTACT | | CCAAUUUUACU |
| HAO1_exon7 | + | CTTA | 370 | GTGTTTGCTACCTCCAA | 825 | GUGUUUGCUACCUCCAAUU |
| | | | | TTTTACTAAAGGA | | UUACUAAAGGA |
| HAO1_exon7 | + | GTTT | 371 | GCTACCTCCAATTTTAC | 826 | GCUACCUCCAAUUUUACUA |
| | | | | TAAAGGATACAGC | | AAGGAUACAGC |
| HAO1_exon7 | + | TTTG | 372 | CTACCTCCAATTTTACT | 827 | CUACCUCCAAUUUUACUAA |
| | | | | AAAGGATACAGCA | | AGGAUACAGCA |
| HAO1_exon7 | + | ATTT | 373 | TACTAAAGGATACAGCA | 828 | UACUAAAGGAUACAGCACU |
| | | | | CTTTAGCCTGCCA | | UUAGCCUGCCA |
| HAO1_exon7 | + | TTTT | 374 | ACTAAAGGATACAGCAC | 829 | ACUAAAGGAUACAGCACUU |
| | | | | TTTAGCCTGCCAG | | UAGCCUGCCAG |
| HAO1_exon7 | + | TTTA | 375 | CTAAAGGATACAGCACT | 830 | CUAAAGGAUACAGCACUUU |
| | | | | TTAGCCTGCCAGG | | AGCCUGCCAGG |
| HAO1_exon7 | + | CTTT | 376 | AGCCTGCCAGGGGATGA | 831 | AGCCUGCCAGGGGAUGACG |
| | | | | CGTTGTCTAAACA | | UUGUCUAAACA |
| HAO1_exon7 | + | TTTA | 377 | GCCTGCCAGGGGATGAC | 832 | GCCUGCCAGGGGAUGACGU |
| | | | | GTTGTCTAAACAC | | UGUCUAAACAC |
| HAO1_exon7 | + | TTTT | 378 | CACCTTAGTGTTTGCTA | 833 | CACCUUAGUGUUUGCUACC |
| | | | | CCTCCAATTTTAC | | UCCAAUUUUAC |
| HAO1_exon7 | + | GTTG | 379 | TCTAAACACATTTTCAA | 834 | UCUAAACACAUUUUCAAUG |
| | | | | TGTTTTCTTTTTA | | UUUUCUUUUUA |
| HAO1_exon7 | + | TTTT | 380 | CAATGTTTTCTTTTTAA | 835 | CAAUGUUUUCUUUUUAACA |
| | | | | CAGTTAATATATT | | GUUAAUAUAUU |
| HAO1_exon7 | + | TTTC | 381 | AATGTTTTCTTTTTAAC | 836 | AAUGUUUUCUUUUUAACAG |
| | | | | AGTTAATATATTT | | UUAAUAUAUUU |
| HAO1_exon7 | + | GTTT | 382 | TCTTTTTAACAGTTAAT | 837 | UCUUUUUAACAGUUAAUAU |
| | | | | ATATTTCCAGGAT | | AUUUCCAGGAU |
| HAO1_exon7 | + | TTTT | 383 | CTTTTTAACAGTTAATA | 838 | CUUUUUAACAGUUAAUAUA |
| | | | | TATTTCCAGGATG | | UUUCCAGGAUG |
| HAO1_exon7 | + | TTTC | 384 | TTTTTAACAGTTAATAT | 839 | UUUUUAACAGUUAAUAUAU |
| | | | | ATTTCCAGGATGA | | UUCCAGGAUGA |
| HAO1_exon7 | + | CTTT | 385 | TTAACAGTTAATATATT | 840 | UUAACAGUUAAUAUAUUUC |
| | | | | TCCAGGATGAAAG | | CAGGAUGAAAG |
| HAO1_exon7 | + | TTTT | 386 | TAACAGTTAATATATTT | 841 | UAACAGUUAAUAUAUUUCC |
| | | | | CCAGGATGAAAGT | | AGGAUGAAAGU |
| HAO1_exon7 | + | TTTT | 387 | AACAGTTAATATATTTC | 842 | AACAGUUAAUAUAUUUCCA |
| | | | | CAGGATGAAAGTC | | GGAUGAAAGUC |
| HAO1_exon7 | + | TTTA | 388 | ACAGTTAATATATTTCC | 843 | ACAGUUAAUAUAUUUCCAG |
| | | | | AGGATGAAAGTCC | | GAUGAAAGUCC |
| HAO1_exon7 | + | ATTT | 389 | TCAATGTTTTCTTTTTA | 844 | UCAAUGUUUUCUUUUUAAC |
| | | | | ACAGTTAATATAT | | AGUUAAUAUAU |
| HAO1_exon7 | + | GTTA | 390 | ATATATTTCCAGGATGA | 845 | AUAUAUUUCCAGGAUGAAA |
| | | | | AAGTCCATTTCTT | | GUCCAUUUCUU |
| HAO1_exon7 | + | CTTT | 391 | TCACCTTAGTGTTTGCT | 846 | UCACCUUAGUGUUUGCUAC |
| | | | | ACCTCCAATTTTA | | CUCCAAUUUUA |
| HAO1_exon7 | + | GTTA | 392 | ATAAACAATGAGATCAT | 847 | AUAAACAAUGAGAUCAUUA |
| | | | | TATCTTTTCACCT | | UCUUUUCACCU |
| HAO1_exon7 | + | TTTC | 393 | TCTCTAAGAAGTAACAT | 848 | UCUCUAAGAAGUAACAUAC |
| | | | | ACATCCTAAAACA | | AUCCUAAAACA |
| HAO1_exon7 | + | ATTT | 394 | GGATATATTCAGACACT | 849 | GGAUAUAUUCAGACACUAA |
| | | | | AAAGATGTGATTG | | AGAUGUGAUUG |
| HAO1_exon7 | + | TTTG | 395 | GATATATTCAGACACTA | 850 | GAUAUAUUCAGACACUAAA |
| | | | | AAGATGTGATTGG | | GAUGUGAUUGG |
| HAO1_exon7 | + | ATTC | 396 | AGACACTAAAGATGTGA | 851 | AGACACUAAAGAUGUGAUU |
| | | | | TTGGAAATCTACA | | GGAAAUCUACA |
| HAO1_exon7 | + | ATTG | 397 | GAAATCTACATTCAAAG | 852 | GAAAUCUACAUUCAAAGAA |
| | | | | AAGTATCACCAAT | | GUAUCACCAAU |
| HAO1_exon7 | + | ATTC | 398 | AAAGAAGTATCACCAAT | 853 | AAAGAAGUAUCACCAAUUA |
| | | | | TACCGCCACCCAT | | CCGCCACCCAU |
| HAO1_exon7 | + | ATTA | 399 | CCGCCACCCATTCCAAT | 854 | CCGCCACCCAUUCCAAUUC |
| | | | | TCTCTCCAGTGCT | | UCUCCAGUGCU |
| HAO1_exon7 | + | ATTC | 400 | CAATTCTCTCCAGTGCT | 855 | CAAUUCUCUCCAGUGCUAC |
| | | | | ACCTTCTCAAAGT | | CUUCUCAAAGU |
| HAO1_exon7 | + | ATTC | 401 | TCTCCAGTGCTACCTTC | 856 | UCUCCAGUGCUACCUUCUC |
| | | | | TCAAAGTTGTGAA | | AAAGUUGUGAA |
| HAO1_exon7 | + | ATTA | 402 | TCTTTTCACCTTAGTGT | 857 | UCUUUUCACCUUAGUGUUU |
| | | | | TTGCTACCTCCAA | | GCUACCUCCAA |
| HAO1_exon7 | + | CTTC | 403 | TCAAAGTTGTGAATCAG | 858 | UCAAAGUUGUGAAUCAGGC |
| | | | | GCATTACCAACAC | | AUUACCAACAC |
| HAO1_exon7 | + | ATTA | 404 | CCAACACTTTGAACCTG | 859 | CCAACACUUUGAACCUGAG |
| | | | | AGCTTACAATTTA | | CUUACAAUUUA |
| HAO1_exon7 | + | CTTT | 405 | GAACCTGAGCTTACAAT | 860 | GAACCUGAGCUUACAAUUU |
| | | | | TTAAGAACCACTG | | AAGAACCACUG |
| HAO1_exon7 | + | TTTG | 406 | AACCTGAGCTTACAATT | 861 | AACCUGAGCUUACAAUUUA |
| | | | | TAAGAACCACTGT | | AGAACCACUGU |
| HAO1_exon7 | + | CTTA | 407 | CAATTTAAGAACCACTG | 862 | CAAUUUAAGAACCACUGUU |
| | | | | TTTTAAAGACATG | | UUAAAGACAUG |
| HAO1_exon7 | + | ATTT | 408 | AAGAACCACTGTTTTAA | 863 | AAGAACCACUGUUUUAAAG |
| | | | | AGACATGTAAACA | | ACAUGUAAACA |
| HAO1_exon7 | + | TTTA | 409 | AGAACCACTGTTTTAAA | 864 | AGAACCACUGUUUUAAAGA |
| | | | | GACATGTAAACAG | | CAUGUAAACAG |
| HAO1_exon7 | + | GTTT | 410 | TAAAGACATGTAAACAG | 865 | UAAAGACAUGUAAACAGAA |
| | | | | AATACAGGTTAAT | | UACAGGUUAAU |
| HAO1_exon7 | + | TTTT | 411 | AAAGACATGTAAACAGA | 866 | AAAGACAUGUAAACAGAAU |
| | | | | ATACAGGTTAATA | | ACAGGUUAAUA |
| HAO1_exon7 | + | TTTA | 412 | AAGACATGTAAACAGAA | 867 | AAGACAUGUAAACAGAAUA |
| | | | | TACAGGTTAATAA | | CAGGUUAAUAA |
| HAO1_exon7 | + | GTTG | 413 | TGAATCAGGCATTACCA | 868 | UGAAUCAGGCAUUACCAAC |
| | | | | ACACTTTGAACCT | | ACUUUGAACCU |
| HAO1_exon7 | − | GTTG | 414 | GTAATGCCTGATTCACA | 869 | GUAAUGCCUGAUUCACAAC |
| | | | | ACTTTGAGAAGGT | | UUUGAGAAGGU |
| HAO1_exon7 | + | ATTT | 415 | CCAGGATGAAAGTCCAT | 870 | CCAGGAUGAAAGUCCAUUU |
| | | | | TTCTTTCTAAAAG | | CUUUCUAAAAG |
| HAO1_exon7 | + | CTTT | 416 | ATTTCTCTCTAAGAAGT | 871 | AUUUCUCUCUAAGAAGUAA |
| | | | | AACATACATCCTA | | CAUACAUCCUA |
| HAO1_exon7 | + | TTTC | 417 | ACATTCTGGCACCCTGA | 872 | ACAUUCUGGCACCCUGAAA |
| | | | | AAAAATAATGCAT | | AAAUAAUGCAU |
| HAO1_exon7 | + | ATTC | 418 | TGGCACCCTGAAAAAAT | 873 | UGGCACCCUGAAAAAAUAA |
| | | | | AATGCATACATTT | | UGCAUACAUUU |
| HAO1_exon7 | + | TTTA | 419 | TTTCTCTCTAAGAAGTA | 874 | UUUCUCUCUAAGAAGUAAC |
| | | | | ACATACATCCTAA | | AUACAUCCUAA |
| HAO1_exon7 | + | CTTC | 420 | CCAAAAATGCTTTATTT | 875 | CCAAAAAUGCUUUAUUUCU |
| | | | | CTCTCTAAGAAGT | | CUCUAAGAAGU |
| HAO1_exon7 | − | CTTC | 421 | TTAGAGAGAAATAAAGC | 876 | UUAGAGAGAAAUAAAGCAU |
| | | | | ATTTTTGGGAAGA | | UUUUGGGAAGA |
| HAO1_exon7 | − | GTTA | 422 | CTTCTTAGAGAGAAATA | 877 | CUUCUUAGAGAGAAAUAAA |
| | | | | AAGCATTTTTGGG | | GCAUUUUUGGG |
| HAO1_exon7 | − | TTTA | 423 | GGATGTATGTTACTTCT | 878 | GGAUGUAUGUUACUUCUUA |
| | | | | TAGAGAGAAATAA | | GAGAGAAAUAA |
| HAO1_exon7 | − | TTTT | 424 | AGGATGTATGTTACTTC | 879 | AGGAUGUAUGUUACUUCUU |
| | | | | TTAGAGAGAAATA | | AGAGAGAAAUA |
| HAO1_exon7 | − | GTTT | 425 | TAGGATGTATGTTACTT | 880 | UAGGAUGUAUGUUACUUCU |
| | | | | CTTAGAGAGAAAT | | UAGAGAGAAAU |
| HAO1_exon7 | + | CTTT | 426 | CACATTCTGGCACCCTG | 881 | CACAUUCUGGCACCCUGAA |
| | | | | AAAAAATAATGCA | | AAAAUAAUGCA |
| HAO1_exon7 | − | TTTA | 427 | GTGTCTGAATATATCCA | 882 | GUGUCUGAAUAUAUCCAAA |
| | | | | AATGTTTTAGGAT | | UGUUUUAGGAU |
| HAO1_exon7 | − | TTTC | 428 | CAATCACATCTTTAGTG | 883 | CAAUCACAUCUUUAGUGUC |
| | | | | TCTGAATATATCC | | UGAAUAUAUCC |
| HAO1_exon7 | − | ATTT | 429 | CCAATCACATCTTTAGT | 884 | CCAAUCACAUCUUUAGUGU |
| | | | | GTCTGAATATATC | | CUGAAUAUAUC |
| HAO1_exon7 | − | TTTG | 430 | AATGTAGATTTCCAATC | 885 | AAUGUAGAUUUCCAAUCAC |
| | | | | ACATCTTTAGTGT | | AUCUUUAGUGU |
| HAO1_exon7 | − | CTTT | 431 | GAATGTAGATTTCCAAT | 886 | GAAUGUAGAUUUCCAAUCA |
| | | | | CACATCTTTAGTG | | CAUCUUUAGUG |
| HAO1_exon7 | − | CTTC | 432 | TTTGAATGTAGATTTCC | 887 | UUUGAAUGUAGAUUUCCAA |
| | | | | AATCACATCTTTA | | UCACAUCUUUA |
| HAO1_exon7 | − | ATTG | 433 | GTGATACTTCTTTGAAT | 888 | GUGAUACUUCUUUGAAUGU |
| | | | | GTAGATTTCCAAT | | AGAUUUCCAAU |
| HAO1_exon7 | − | ATTG | 434 | GAATGGGTGGCGGTAAT | 889 | GAAUGGGUGGCGGUAAUUG |
| | | | | TGGTGATACTTCT | | GUGAUACUUCU |
| HAO1_exon7 | − | TTTG | 435 | AGAAGGTAGCACTGGAG | 890 | AGAAGGUAGCACUGGAGAG |
| | | | | AGAATTGGAATGG | | AAUUGGAAUGG |
| HAO1_exon7 | − | CTTT | 436 | GAGAAGGTAGCACTGGA | 891 | GAGAAGGUAGCACUGGAGA |
| | | | | GAGAATTGGAATG | | GAAUUGGAAUG |
| HAO1_exon7 | − | CTTT | 437 | AGTGTCTGAATATATCC | 892 | AGUGUCUGAAUAUAUCCAA |
| | | | | AAATGTTTTAGGA | | AUGUUUUAGGA |
| HAO1_exon7 | + | TTTC | 438 | CAGGATGAAAGTCCATT | 893 | CAGGAUGAAAGUCCAUUUC |
| | | | | TCTTTCTAAAAGG | | UUUCUAAAAGG |
| HAO1_exon7 | + | CTTG | 439 | TCGATGACTTTCACATT | 894 | UCGAUGACUUUCACAUUCU |
| | | | | CTGGCACCCTGAA | | GGCACCCUGAA |
| HAO1_exon7 | + | TTTT | 440 | CCTCACCAATGTCTTGT | 895 | CCUCACCAAUGUCUUGUCG |
| | | | | CGATGACTTTCAC | | AUGACUUUCAC |
| HAO1_exon7 | + | TTTC | 441 | TTTCTAAAAGGTTCCTA | 896 | UUUCUAAAAGGUUCCUAGG |
| | | | | GGACACCCATTGA | | ACACCCAUUGA |
| HAO1_exon7 | + | CTTT | 442 | CTAAAAGGTTCCTAGGA | 897 | CUAAAAGGUUCCUAGGACA |
| | | | | CACCCATTGAAAA | | CCCAUUGAAAA |
| HAO1_exon7 | + | TTTC | 443 | TAAAAGGTTCCTAGGAC | 898 | UAAAAGGUUCCUAGGACAC |
| | | | | ACCCATTGAAAAG | | CCAUUGAAAAG |
| HAO1_exon7 | + | GTTC | 444 | CTAGGACACCCATTGAA | 899 | CUAGGACACCCAUUGAAAA |
| | | | | AAGTCAAAAGCAA | | GUCAAAAGCAA |
| HAO1_exon7 | + | ATTG | 445 | AAAAGTCAAAAGCAATG | 900 | AAAAGUCAAAAGCAAUGAA |
| | | | | AAATAAAAGGGAT | | AUAAAAGGGAU |
| HAO1_exon7 | + | ATTG | 446 | CTATTTTGTTGGAAAAG | 901 | CUAUUUUGUUGGAAAAGAA |
| | | | | AACGACACCCTTT | | CGACACCCUUU |
| HAO1_exon7 | + | ATTT | 447 | TGTTGGAAAAGAACGAC | 902 | UGUUGGAAAAGAACGACAC |
| | | | | ACCCTTTGTATTG | | CCUUUGUAUUG |
| HAO1_exon7 | + | TTTT | 448 | GTTGGAAAAGAACGACA | 903 | GUUGGAAAAGAACGACACC |
| | | | | CCCTTTGTATTGA | | CUUUGUAUUGA |
| HAO1_exon7 | + | TTTG | 449 | TTGGAAAAGAACGACAC | 904 | UUGGAAAAGAACGACACCC |
| | | | | CCTTTGTATTGAA | | UUUGUAUUGAA |
| HAO1_exon7 | + | TTTC | 450 | CTCACCAATGTCTTGTC | 905 | CUCACCAAUGUCUUGUCGA |
| | | | | GATGACTTTCACA | | UGACUUUCACA |
| HAO1_exon7 | + | GTTG | 451 | GAAAAGAACGACACCCT | 906 | GAAAAGAACGACACCCUUU |
| | | | | TTGTATTGAAGTG | | GUAUUGAAGUG |
| HAO1_exon7 | + | TTTG | 452 | TATTGAAGTGGGGAATT | 907 | UAUUGAAGUGGGGAAUUAC |
| | | | | ACAGACTGTGGTC | | AGACUGUGGUC |
| HAO1_exon7 | + | ATTG | 453 | AAGTGGGGAATTACAGA | 908 | AAGUGGGGAAUUACAGACU |
| | | | | CTGTGGTCACCCT | | GUGGUCACCCU |
| HAO1_exon7 | + | ATTA | 454 | CAGACTGTGGTCACCCT | 909 | CAGACUGUGGUCACCCUCU |
| | | | | CTGCACAGTGTCT | | GCACAGUGUCU |
| HAO1_exon7 | + | CTTT | 455 | GTCAAGTAATACATGCT | 910 | GUCAAGUAAUACAUGCUGA |
| | | | | GAAAAAAAATAAT | | AAAAAAAUAAU |
| HAO1_exon7 | + | TTTG | 456 | TCAAGTAATACATGCTG | 911 | UCAAGUAAUACAUGCUGAA |
| | | | | AAAAAAAATAATA | | AAAAAAUAAUA |
| HAO1_exon7 | + | ATTG | 457 | TGCACTGTCAGATCTTG | 912 | UGCACUGUCAGAUCUUGGA |
| | | | | GAAACGGCCAAAG | | AACGGCCAAAG |
| HAO1_exon7 | + | CTTG | 458 | GAAACGGCCAAAGGATT | 913 | GAAACGGCCAAAGGAUUUU |
| | | | | TTTCCTCACCAAT | | UCCUCACCAAU |
| HAO1_exon7 | + | ATTT | 459 | TTCCTCACCAATGTCTT | 914 | UUCCUCACCAAUGUCUUGU |
| | | | | GTCGATGACTTTC | | CGAUGACUUUC |
| HAO1_exon7 | + | TTTT | 460 | TCCTCACCAATGTCTTG | 915 | UCCUCACCAAUGUCUUGUC |
| | | | | TCGATGACTTTCA | | GAUGACUUUCA |
| HAO1_exon7 | + | CTTT | 461 | GTATTGAAGTGGGGAAT | 916 | GUAUUGAAGUGGGGAAUUA |
| | | | | TACAGACTGTGGT | | CAGACUGUGGU |
| HAO1_exon7 | + | ATTT | 462 | CTTTCTAAAAGGTTCCT | 917 | CUUUCUAAAAGGUUCCUAG |
| | | | | AGGACACCCATTG | | GACACCCAUUG |
| HAO1_exon7 | − | CTTA | 463 | GAGAGAAATAAAGCATT | 918 | GAGAGAAAUAAAGCAUUUU |
| | | | | TTTGGGAAGAA | | UGGGAAGAA |
| HAO1_exon7 | + | ATTT | 464 | AATATGAACTGAATGCA | 919 | AAUAUGAACUGAAUGCAAU |
| | | | | ATAATAATCA | | AAUAAUCA |
| HAO1_exon7 | + | TTTA | 465 | ATATGAACTGAATGCAA | 920 | AUAUGAACUGAAUGCAAUA |
| | | | | TAATAATCA | | AUAAUCA |

*The 5′-TTN-3′ 3-nucleotide PAM motif is in boldface.

The present disclosure includes all combinations of the direct repeats and spacers listed above, consistent with the disclosure herein.

In some embodiments, a spacer sequence described herein comprises an uracil (U). In some embodiments, a spacer sequence described herein comprises a thymine (T). In some embodiments, a spacer sequence according to Table 5 comprises a sequence comprising a thymine in one or more places indicated as uracil in Table 5.

(iii). Exemplary RNA Guides

The present disclosure provides RNA guides that comprise any and all combinations of the direct repeats and spacers described herein (e.g., as set forth in Table 5, above). In some embodiments, the sequence of an RNA guide has at least 90% identity (e.g., at least 90%, 91%, 92%, 93%, 94%, 95%, 96%, 97%, 98%, or 99% identity) to a sequence of any one of SEQ ID NOs: 967-1023. In some embodiments, an RNA guide has a sequence of any one of SEQ ID NOs: 967-1023.

In some embodiments, exemplary RNA guides provided herein may comprise a spacer sequence of any one of SEQ ID NOs: 1093-1097. In one example, the RNA guide may comprise a spacer of SEQ ID NO: 1096.

Any of the exemplary RNA guides disclosed herein may comprise a direct sequence of any one of SEQ ID NOs: 1-10 or a fragment thereof that is at least 23-nucleotide in length. In one example, the direct sequence may comprise SEQ ID NO: 10.

In specific examples, the RNA guides provide herein may comprise the nucleotide sequence of SEQ ID NOs: 967, 968, 988, 989, or 994. In one example, the RNA guide provided herein comprise the nucleotide sequence of SEQ ID NO: 989.

### (iv). Modifications

The RNA guide may include one or more covalent modifications with respect to a reference sequence, in particular the parent polyribonucleotide, which are included within the scope of the present disclosure.

Exemplary modifications can include any modification to the sugar, the nucleobase, the internucleoside linkage (e.g., to a linking phosphate/to a phosphodiester linkage/to the phosphodiester backbone), and any combination thereof. Some of the exemplary modifications provided herein are described in detail below.

The RNA guide may include any useful modification, such as to the sugar, the nucleobase, or the internucleoside linkage (e.g., to a linking phosphate/to a phosphodiester linkage/to the phosphodiester backbone). One or more atoms of a pyrimidine nucleobase may be replaced or substituted with optionally substituted amino, optionally substituted thiol, optionally substituted alkyl (e.g., methyl or ethyl), or halo (e.g., chloro or fluoro). In certain embodiments, modifications (e.g., one or more modifications) are present in each of the sugar and the internucleoside linkage. Modifications may be modifications of ribonucleic acids (RNAs) to deoxyribonucleic acids (DNAs), threose nucleic acids (TNAs), glycol nucleic acids (GNAs), peptide nucleic acids (PNAs), locked nucleic acids (LNAs) or hybrids thereof). Additional modifications are described herein.

In some embodiments, the modification may include a chemical or cellular induced modification. For example, some nonlimiting examples of intracellular RNA modifications are described by Lewis and Pan in “RNA modifications and structures cooperate to RNA guide-protein interactions” from Nat Reviews Mol Cell Biol, 2017, 18:202-210.

Different sugar modifications, nucleotide modifications, and/or internucleoside linkages (e.g., backbone structures) may exist at various positions in the sequence. One of ordinary skill in the art will appreciate that the nucleotide analogs or other modification(s) may be located at any position(s) of the sequence, such that the function of the sequence is not substantially decreased. The sequence may include from about 1% to about 100% modified nucleotides (either in relation to overall nucleotide content, or in relation to one or more types of nucleotide, i.e., any one or more of A. G. U or C) or any intervening percentage (e.g., from 1% to 20%>, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%).

In some embodiments, sugar modifications (e.g., at the 2′ position or 4′ position) or replacement of the sugar at one or more ribonucleotides of the sequence may, as well as backbone modifications, include modification or replacement of the phosphodiester linkages. Specific examples of a sequence include, but are not limited to, sequences including modified backbones or no natural internucleoside linkages such as internucleoside modifications, including modification or replacement of the phosphodiester linkages. Sequences having modified backbones include, among others, those that do not have a phosphorus atom in the backbone. For the purposes of this application, and as sometimes referenced in the art, modified RNAs that do not have a phosphorus atom in their internucleoside backbone can also be considered to be oligonucleosides. In particular embodiments, a sequence will include ribonucleotides with a phosphorus atom in its internucleoside backbone.

Modified sequence backbones may include, for example, phosphorothioates, chiral phosphorothioates, phosphorodithioates, phosphotriesters, aminoalkylphosphotriesters, methyl and other alkyl phosphonates such as 3′-alkylene phosphonates and chiral phosphonates, phosphinates, phosphoramidates such as 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included. In some embodiments, the sequence may be negatively or positively charged.

The modified nucleotides, which may be incorporated into the sequence, can be modified on the internucleoside linkage (e.g., phosphate backbone). Herein, in the context of the polynucleotide backbone, the phrases “phosphate” and “phosphodiester” are used interchangeably. Backbone phosphate groups can be modified by replacing one or more of the oxygen atoms with a different substituent. Further, the modified nucleosides and nucleotides can include the wholesale replacement of an unmodified phosphate moiety with another internucleoside linkage as described herein. Examples of modified phosphate groups include, but are not limited to, phosphorothioate, phosphoroselenates, boranophosphates, boranophosphate esters, hydrogen phosphonates, phosphoramidates, phosphorodiamidates, alkyl or aryl phosphonates, and phosphotriesters. Phosphorodithioates have both non-linking oxygens replaced by sulfur. The phosphate linker can also be modified by the replacement of a linking oxygen with nitrogen (bridged phosphoramidates), sulfur (bridged phosphorothioates), and carbon (bridged methylene-phosphonates).

The α-thio substituted phosphate moiety is provided to confer stability to RNA and DNA polymers through the unnatural phosphorothioate backbone linkages. Phosphorothioate DNA and RNA have increased nuclease resistance and subsequently a longer half-life in a cellular environment.

In specific embodiments, a modified nucleoside includes an alpha-thio-nucleoside (e.g., 5′-O-(1-thiophosphate)-adenosine, 5′-O-(1-thiophosphate)-cytidine (a-thio-cytidine), 5′-O-(1-thiophosphate)-guanosine, 5′-O-(1-thiophosphate)-uridine, or 5′-O-(1-thiophosphate)-pseudouridine).

Other internucleoside linkages that may be employed according to the present disclosure, including internucleoside linkages which do not contain a phosphorous atom, are described herein.

In some embodiments, the sequence may include one or more cytotoxic nucleosides. For example, cytotoxic nucleosides may be incorporated into sequence, such as bifunctional modification. Cytotoxic nucleoside may include, but are not limited to, adenosine arabinoside, 5-azacytidine, 4′-thio-aracytidine, cyclopentenylcytosine, cladribine, clofarabine, cytarabine, cytosine arabinoside, 1-(2-C-cyano-2-deoxy-beta-D-arabino-pentofuranosyl)-cytosine, decitabine, 5-fluorouracil, fludarabine, floxuridine, gemcitabine, a combination of tegafur and uracil, tegafur ((RS)-5-fluoro-1-(tetrahydrofuran-2-yl)pyrimidine-2,4(1H,3H)-dione), troxacitabine, tezacitabine, 2′-deoxy-2′-methylidenecytidine (DMDC), and 6-mercaptopurine. Additional examples include fludarabine phosphate, N4-behenoyl-1-beta-D-arabinofuranosylcytosine, N4-octadecyl-1-beta-D-arabinofuranosylcytosine, N4-palmitoyl-1-(2-C-cyano-2-deoxy-beta-D-arabino-pentofuranosyl) cytosine, and P-4055 (cytarabine 5′-elaidic acid ester).

In some embodiments, the sequence includes one or more post-transcriptional modifications (e.g., capping, cleavage, polyadenylation, splicing, poly-A sequence, methylation, acylation, phosphorylation, methylation of lysine and arginine residues, acetylation, and nitrosylation of thiol groups and tyrosine residues, etc). The one or more post-transcriptional modifications can be any post-transcriptional modification, such as any of the more than one hundred different nucleoside modifications that have been identified in RNA (Rozenski, J, Crain, P. and McCloskey, J. (1999). The RNA Modification Database: 1999 update. Nucl Acids Res 27: 196-197) In some embodiments, the first isolated nucleic acid comprises messenger RNA (mRNA). In some embodiments, the mRNA comprises at least one nucleoside selected from the group consisting of pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine. In some embodiments, the mRNA comprises at least one nucleoside selected from the group consisting of 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine. In some embodiments, the mRNA comprises at least one nucleoside selected from the group consisting of 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine. In some embodiments, mRNA comprises at least one nucleoside selected from the group consisting of inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

The sequence may or may not be uniformly modified along the entire length of the molecule. For example, one or more or all types of nucleotides (e.g., naturally-occurring nucleotides, purine or pyrimidine, or any one or more or all of A, G, U, C, I, pU) may or may not be uniformly modified in the sequence, or in a given predetermined sequence region thereof. In some embodiments, the sequence includes a pseudouridine. In some embodiments, the sequence includes an inosine, which may aid in the immune system characterizing the sequence as endogenous versus viral RNAs. The incorporation of inosine may also mediate improved RNA stability/reduced degradation. See for example, Yu, Z. et al. (2015) RNA editing by ADAR1 marks dsRNA as “self”. Cell Res. 25, 1283-1284, which is incorporated by reference in its entirety.

In some embodiments, one or more of the nucleotides of an RNA guide comprises a 2′-O-methyl phosphorothioate modification. In some embodiments, each of the first three nucleotides of the RNA guide comprises a 2′-O-methyl phosphorothioate modification. In some embodiments, each of the last four nucleotides of the RNA guide comprises a 2′-O-methyl phosphorothioate modification. In some embodiments, each of the first to last, second to last, and third to last nucleotides of the RNA guide comprises a 2′-O-methyl phosphorothioate modification, and wherein the last nucleotide of the RNA guide is unmodified. In some embodiments, each of the first three nucleotides of the RNA guide comprises a 2′-O-methyl phosphorothioate modification, and each of the first to last, second to last, and third to last nucleotides of the RNA guide comprises a 2′-O-methyl phosphorothioate modification.

When a gene editing system disclosed herein comprises nucleic acids encoding the Cas12i polypeptide disclosed herein, e.g., mRNA molecules, such nucleic acid molecules may contain any of the modifications disclosed herein, where applicable.

### B. Cas12i Polypeptides

In some embodiments, the composition or system of the present disclosure includes a Cas12i polypeptide as described in WO/2019/178427, the relevant disclosures of which are incorporated by reference for the subject matter and purpose referenced herein.

In some embodiments, the genetic editing system disclosed herein includes a Cas12i2 polypeptide described herein (e.g., a polypeptide comprising SEQ ID NO: 922 and/or encoded by SEQ ID NO: 921). In some embodiments, the Cas12i2 polypeptide comprises at least one RuvC domain.

A nucleic acid sequence encoding the Cas12i2 polypeptide described herein may be substantially identical to a reference nucleic acid sequence, e.g., SEQ ID NO: 921. In some embodiments, the Cas12i2 polypeptide is encoded by a nucleic acid comprising a sequence having least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to the reference nucleic acid sequence, e.g., SEQ ID NO: 921. The percent identity between two such nucleic acids can be determined manually by inspection of the two optimally aligned nucleic acid sequences or by using software programs or algorithms (e.g., BLAST, ALIGN, CLUSTAL) using standard parameters. One indication that two nucleic acid sequences are substantially identical is that the nucleic acid molecules hybridize to the complementary sequence of the other under stringent conditions of temperature and ionic strength (e.g., within a range of medium to high stringency). See, e.g., Tijssen, “Hybridization with Nucleic Acid Probes. Part I. Theory and Nucleic Acid Preparation” (Laboratory Techniques in Biochemistry and Molecular Biology, Vol 24).

In some embodiments, the Cas12i2 polypeptide is encoded by a nucleic acid sequence having at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more sequence identity, but not 100% sequence identity, to a reference nucleic acid sequence, e.g., SEQ ID NO: 921.

In some embodiments, the Cas12i2 polypeptide of the present disclosure comprises a polypeptide sequence having at least 50%, 60%, 65%, 70%, 75%, 80%, 85%, 90%, 91%, 92%, 93%, 94%, 95%, 96%, 97%, 98%, 99%, or 100% identity to SEQ ID NO: 922.

In some embodiments, the present disclosure describes a Cas12i2 polypeptide having a specified degree of amino acid sequence identity to one or more reference polypeptides, e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or even at least 99%, but not 100%, sequence identity to the amino acid sequence of SEQ ID NO: 922. Homology or identity can be determined by amino acid sequence alignment, e.g., using a program such as BLAST, ALIGN, or CLUSTAL, as described herein.

Also provided is a Cas12i2 polypeptide of the present disclosure having enzymatic activity, e.g., nuclease or endonuclease activity, and comprising an amino acid sequence which differs from the amino acid sequences of SEQ ID NO: 922 by 50, 40, 35, 30, 25, 20, 19, 18, 17, 16, 15, 14, 13, 12, 11, 10, 9, 8, 7, 6, 5, 4, 3, 2, 1, or 0 amino acid residue(s), when aligned using any of the previously described alignment methods.

In some examples, the Cas12i2 polypeptide may contain one or more mutations relative to SEQ ID NO: 922, for example, at position D581, G624, F626, P868, I926, V1030, E1035, S1046, or any combination thereof. In some instances, the one or more mutations are amino acid substitutions, for example, D581R, G624R, F626R, P868T, I926R, V1030G, E1035R, S1046G, or a combination thereof.

In some examples, the Cas12i2 polypeptide contains mutations at positions D581, D911, I926, and V1030. Such a Cas12i2 polypeptide may contain amino acid substitutions of D581R, D911R, I926R, and V1030G (e.g., SEQ ID NO: 923). In some examples, the Cas12i2 polypeptide contains mutations at positions D581, I926, and V1030. Such a Cas12i2 polypeptide may contain amino acid substitutions of D581R, I926R, and V1030G (e.g., SEQ ID NO: 924). In some examples, the Cas12i2 polypeptide may contain mutations at positions D581, I926, V1030, and S1046. Such a Cas12i2 polypeptide may contain amino acid substitutions of D581R, I926R, V1030G, and S1046G (e.g., SEQ ID NO: 925). In some examples, the Cas12i2 polypeptide may contain mutations at positions D581, G624, F626, I926, V1030, E1035, and S1046. Such a Cas12i2 polypeptide may contain amino acid substitutions of D581R, G624R, F626R, I926R, V1030G, E1035R, and S1046G (e.g., SEQ ID NO: 926). In some examples, the Cas12i2 polypeptide may contain mutations at positions D581, G624, F626, P868, I926, V1030, E1035, and S1046. Such a Cas12i2 polypeptide may contain amino acid substitutions of D581R, G624R. F626R. P868T, I926R, V1030G, E1035R, and S1046G (e.g., SEQ ID NO: 927).

In some embodiments, the Cas12i2 polypeptide of the present disclosure comprises a polypeptide sequence having at least 50%, 60%, 65%, 70%, 75%, 80%, 85%, 90%, 91%, 92%, 93%, 94%, 95%, 96%, 97%, 98%, 99%, or 100% identity to SEQ ID NO: 923, SEQ ID NO: 924, SEQ ID NO: 925, SEQ ID NO: 926, or SEQ ID NO: 927. In some embodiments, a Cas12i2 polypeptide having at least 50%, 60%, 65%, 70%, 75%, 80%, 85%, 90%, 91%, 92%, 93%, 94%, 95%, 96%, 97%, 98%, 99%, or 100% identity to SEQ ID NO: 923, SEQ ID NO: 924, SEQ ID NO: 925, SEQ ID NO: 926, or SEQ ID NO: 927 maintains the amino acid changes (or at least 1, 2, 3 etc. of these changes) that differentiate the polypeptide from its respective parent/reference sequence.

In some embodiments, the present disclosure describes a Cas12i2 polypeptide having a specified degree of amino acid sequence identity to one or more reference polypeptides, e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or even at least 99%, but not 100%, sequence identity to the amino acid sequence of SEQ ID NO: 923, SEQ ID NO: 924, SEQ ID NO: 925, SEQ ID NO: 926, or SEQ ID NO: 927. Homology or identity can be determined by amino acid sequence alignment, e.g., using a program such as BLAST, ALIGN, or CLUSTAL, as described herein.

Also provided is a Cas12i2 polypeptide of the present disclosure having enzymatic activity, e.g., nuclease or endonuclease activity, and comprising an amino acid sequence which differs from the amino acid sequences of SEQ ID NO: 923, SEQ ID NO: 924, SEQ ID NO: 925, SEQ ID NO: 926, or SEQ ID NO: 927 by 50, 40, 35, 30, 25, 20, 19, 18, 17, 16, 15, 14, 13, 12, 11, 10, 9, 8, 7, 6, 5, 4, 3, 2, 1, or 0 amino acid residue(s), when aligned using any of the previously described alignment methods.

In some embodiments, the composition of the present disclosure includes a Cas1214 polypeptide described herein (e.g., a polypeptide comprising SEQ ID NO: 956 and/or encoded by SEQ ID NO: 955). In some embodiments, the Cas12i4 polypeptide comprises at least one RuvC domain.

A nucleic acid sequence encoding the Cas12i4 polypeptide described herein may be substantially identical to a reference nucleic acid sequence, e.g., SEQ ID NO: 955. In some embodiments, the Cas1214 polypeptide is encoded by a nucleic acid comprising a sequence having least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to the reference nucleic acid sequence, e.g., SEQ ID NO: 955. The percent identity between two such nucleic acids can be determined manually by inspection of the two optimally aligned nucleic acid sequences or by using software programs or algorithms (e.g., BLAST, ALIGN, CLUSTAL) using standard parameters. One indication that two nucleic acid sequences are substantially identical is that the nucleic acid molecules hybridize to the complementary sequence of the other under stringent conditions of temperature and ionic strength (e.g., within a range of medium to high stringency).

In some embodiments, the Cas1214 polypeptide is encoded by a nucleic acid sequence having at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more sequence identity, but not 100% sequence identity, to a reference nucleic acid sequence, e.g., SEQ ID NO: 955.

In some embodiments, the Cas1214 polypeptide of the present disclosure comprises a polypeptide sequence having at least 50%, 60%, 65%, 70%, 75%, 80%, 85%, 90%, 91%, 92%, 93%, 94%, 95%, 96%, 97%, 98%, 99%, or 100% identity to SEQ ID NO: 956.

In some embodiments, the present disclosure describes a Cas12i4 polypeptide having a specified degree of amino acid sequence identity to one or more reference polypeptides, e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or even at least 99%, but not 100%, sequence identity to the amino acid sequence of SEQ ID NO: 956. Homology or identity can be determined by amino acid sequence alignment, e.g., using a program such as BLAST, ALIGN, or CLUSTAL, as described herein.

Also provided is a Cas12i4 polypeptide of the present disclosure having enzymatic activity, e.g., nuclease or endonuclease activity, and comprising an amino acid sequence which differs from the amino acid sequences of SEQ ID NO: 956 by 50, 40, 35, 30, 25, 20, 19, 18, 17, 16, 15, 14, 13, 12, 11, 10, 9, 8, 7, 6, 5, 4, 3, 2, 1, or 0 amino acid residue(s), when aligned using any of the previously described alignment methods.

In some embodiments, the Cas12i4 polypeptide comprises a polypeptide having a sequence of SEQ ID NO: 957 or SEQ ID NO: 958.

In some embodiments, the Cas1214 polypeptide of the present disclosure comprises a polypeptide sequence having at least 50%, 60%, 65%, 70%, 75%, 80%, 85%, 90%, 91%, 92%, 93%, 94%, 95%, 96%, 97%, 98%, 99%, or 100% identity to SEQ ID NO: 957 or SEQ ID NO: 958. In some embodiments, a Cas1214 polypeptide having at least 50%, 60%, 65%, 70%, 75%, 80%, 85%, 90%, 91%, 92%, 93%, 94%, 95%, 96%, 97%, 98%, 99%, or 100% identity to SEQ ID NO: 957 or SEQ ID NO: 958 maintains the amino acid changes (or at least 1, 2, 3 etc. of these changes) that differentiate it from its respective parent/reference sequence.

In some embodiments, the present disclosure describes a Cas1214 polypeptide having a specified degree of amino acid sequence identity to one or more reference polypeptides, e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or even at least 99%, but not 100%, sequence identity to the amino acid sequence of SEQ ID NO: 957 or SEQ ID NO: 958. Homology or identity can be determined by amino acid sequence alignment, e.g., using a program such as BLAST, ALIGN, or CLUSTAL, as described herein.

Also provided is a Cas12i4 polypeptide of the present disclosure having enzymatic activity, e.g., nuclease or endonuclease activity, and comprising an amino acid sequence which differs from the amino acid sequences of SEQ ID NO: 957 or SEQ ID NO: 958 by 50, 40, 35, 30, 25, 20, 19, 18, 17, 16, 15, 14, 13, 12, 11, 10, 9, 8, 7, 6, 5, 4, 3, 2, 1, or 0 amino acid residue(s), when aligned using any of the previously described alignment methods.

In some embodiments, the composition of the present disclosure includes a Cas12i1 polypeptide described herein (e.g., a polypeptide comprising SEQ ID NO: 965). In some embodiments, the Cas12i4 polypeptide comprises at least one RuvC domain.

In some embodiments, the Cas12i1 polypeptide of the present disclosure comprises a polypeptide sequence having at least 50%, 60%, 65%, 70%, 75%, 80%, 85%, 90%, 91%, 92%, 93%, 94%, 95%, 96%, 97%, 98%, 99%, or 100% identity to SEQ ID NO: 965.

In some embodiments, the present disclosure describes a Cas12i1 polypeptide having a specified degree of amino acid sequence identity to one or more reference polypeptides, e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or even at least 99%, but not 100%, sequence identity to the amino acid sequence of SEQ ID NO: 965. Homology or identity can be determined by amino acid sequence alignment, e.g., using a program such as BLAST, ALIGN, or CLUSTAL, as described herein.

Also provided is a Cas12i1 polypeptide of the present disclosure having enzymatic activity, e.g., nuclease or endonuclease activity, and comprising an amino acid sequence which differs from the amino acid sequences of SEQ ID NO: 965 by 50, 40, 35, 30, 25, 20, 19, 18, 17, 16, 15, 14, 13, 12, 11, 10, 9, 8, 7, 6, 5, 4, 3, 2, 1, or 0 amino acid residue(s), when aligned using any of the previously described alignment methods.

In some embodiments, the composition of the present disclosure includes a Cas1213 polypeptide described herein (e.g., a polypeptide comprising SEQ ID NO: 966). In some embodiments, the Cas12i4 polypeptide comprises at least one RuvC domain.

In some embodiments, the Cas1213 polypeptide of the present disclosure comprises a polypeptide sequence having at least 50%, 60%, 65%, 70%, 75%, 80%, 85%, 90%, 91%, 92%, 93%, 94%, 95%, 96%, 97%, 98%, 99%, or 100% identity to SEQ ID NO: 966.

In some embodiments, the present disclosure describes a Cas1213 polypeptide having a specified degree of amino acid sequence identity to one or more reference polypeptides, e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or even at least 99%, but not 100%, sequence identity to the amino acid sequence of SEQ ID NO: 966. Homology or identity can be determined by amino acid sequence alignment, e.g., using a program such as BLAST, ALIGN, or CLUSTAL, as described herein.

Also provided is a Cas1213 polypeptide of the present disclosure having enzymatic activity, e.g., nuclease or endonuclease activity, and comprising an amino acid sequence which differs from the amino acid sequences of SEQ ID NO: 966 by 50, 40, 35, 30, 25, 20, 19, 18, 17, 16, 15, 14, 13, 12, 11, 10, 9, 8, 7, 6, 5, 4, 3, 2, 1, or 0 amino acid residue(s), when aligned using any of the previously described alignment methods.

Although the changes described herein may be one or more amino acid changes, changes to the Cas12i polypeptide may also be of a substantive nature, such as fusion of polypeptides as amino- and/or carboxyl-terminal extensions. For example, the Cas12i polypeptide may contain additional peptides, e.g., one or more peptides. Examples of additional peptides may include epitope peptides for labelling, such as a polyhistidine tag (His-tag), Myc, and FLAG. In some embodiments, the Cas12i polypeptide described herein can be fused to a detectable moiety such as a fluorescent protein (e.g., green fluorescent protein (GFP) or yellow fluorescent protein (YFP)).

In some embodiments, the Cas12i polypeptide comprises at least one (e.g., two, three, four, five, six, or more) nuclear localization signal (NLS). In some embodiments, the Cas12i polypeptide comprises at least one (e.g., two, three, four, five, six, or more) nuclear export signal (NES). In some embodiments, the Cas12i polypeptide comprises at least one (e.g., two, three, four, five, six, or more) NLS and at least one (e.g., two, three, four, five, six, or more) NES.

In some embodiments, the Cas12i polypeptide described herein can be self-inactivating. See, Epstein et al., “Engineering a Self-Inactivating CRISPR System for AAV Vectors,” Mol. Ther., 24 (2016): S50, which is incorporated by reference in its entirety.

In some embodiments, the nucleotide sequence encoding the Cas12i polypeptide described herein can be codon-optimized for use in a particular host cell or organism. For example, the nucleic acid can be codon-optimized for any non-human eukaryote including mice, rats, rabbits, dogs, livestock, or non-human primates. Codon usage tables are readily available, for example, at the “Codon Usage Database” available at www.kazusa.orjp/codon/and these tables can be adapted in a number of ways. See Nakamura et al. Nucl. Acids Res. 28:292 (2000), which is incorporated herein by reference in its entirety. Computer algorithms for codon optimizing a particular sequence for expression in a particular host cell are also available, such as Gene Forge (Aptagen; Jacobus, PA). In some examples, the nucleic acid encoding the Cas12i polypeptides such as Cas12i2 polypeptides as disclosed herein can be an mRNA molecule, which can be codon optimized.

Exemplary Cas12i polypeptide sequences and corresponding nucleotide sequences are listed in Table 6.

TABLE 6
Cas12i and HAO1 Sequences

| SEQ ID | | |
|---|---|---|
| NO | Sequence | Description |
| 921 | ATGAGCAGCGCGATCAAAAGCTACAAGAGCGTTCTGCGTCCGAACGAGCGTAAGAA | Nucleotide |
| | CCAACTGCTGAAAAGCACCATTCAGTGCCTGGAAGACGGTAGCGCGTTCTTTTTCA | sequence |
| | AGATGCTGCAAGGCCTGTTTGGTGGCATCACCCCGGAGATTGTTCGTTTCAGCACC | encoding |
| | GAACAGGAGAAACAGCAACAGGATATCGCGCTGTGGTGCGCGGTTAACTGGTTCCG | parent |
| | TCCGGTGAGCCAAGACAGCCTGACCCACACCATTGCGAGCGATAACCTGGTGGAGA | Cas 1212 |
| | AGTTTGAGGAATACTATGGTGGCACCGCGAGCGACGCGATCAAACAGTACTTCAGC | |
| | GCGAGCATTGGCGAAAGCTACTATTGGAACGACTGCCGTCAACAGTACTATGATCT | |
| | GTGCCGTGAGCTGGGTGTTGAGGTGAGCGACCTGACCCATGATCTGGAGATCCTGT | |
| | GCCGTGAAAAGTGCCTGGCGGTTGCGACCGAGAGCAACCAGAACAACAGCATCATT | |
| | AGCGTTCTGTTTGGCACCGGCGAAAAAGAGGACCGTAGCGTGAAACTGCGTATCAC | |
| | CAAGAAAATTCTGGAGGCGATCAGCAACCTGAAAGAAATCCCGAAGAACGTTGCGC | |
| | CGATTCAAGAGATCATTCTGAACGTGGCGAAAGCGACCAAGGAAACCTTCCGTCAG | |
| | GTGTATGCGGGTAACCTGGGTGCGCCGAGCACCCTGGAGAAATTTATCGCGAAGGA | |
| | CGGCCAAAAAGAGTTCGATCTGAAGAAACTGCAGACCGACCTGAAGAAAGTTATTC | |
| | GTGGTAAAAGCAAGGAGCGTGATTGGTGCTGCCAGGAAGAGCTGCGTAGCTACGTG | |
| | GAGCAAAACACCATCCAGTATGACCTGTGGGCGTGGGGCGAAATGTTCAACAAAGC | |
| | GCACACCGCGCTGAAAATCAAGAGCACCCGTAACTACAACTTTGCGAAGCAACGTC | |
| | TGGAACAGTTCAAAGAGATTCAGAGCCTGAACAACCTGCTGGTTGTGAAGAAGCTG | |
| | AACGACTTTTTCGATAGCGAATTTTTCAGCGGCGAGGAAACCTACACCATCTGCGT | |
| | TCACCATCTGGGTGGCAAGGACCTGAGCAAACTGTATAAGGCGTGGGAGGATGATC | |
| | CGGCGGACCCGGAAAACGCGATTGTGGTTCTGTGCGACGATCTGAAAAACAACTTT | |
| | AAGAAAGAGCCGATCCGTAACATTCTGCGTTACATCTTCACCATTCGTCAAGAATG | |
| | CAGCGCGCAGGACATCCTGGCGGCGGCGAAGTACAACCAACAGCTGGATCGTTATA | |
| | AAAGCCAAAAGGCGAACCCGAGCGTTCTGGGTAACCAGGGCTTTACCTGGACCAAC | |
| | GCGGTGATCCTGCCGGAGAAGGCGCAGCGTAACGACCGTCCGAACAGCCTGGATCT | |
| | GCGTATTTGGCTGTACCTGAAACTGCGTCACCCGGACGGTCGTTGGAAGAAACACC | |
| | ATATCCCGTTCTACGATACCCGTTTCTTCCAAGAAATTTATGCGGCGGGCAACAGC | |
| | CCGGTTGACACCTGCCAGTTTCGTACCCCGCGTTTCGGTTATCACCTGCCGAAACT | |
| | GACCGATCAGACCGCGATCCGTGTTAACAAGAAACATGTGAAAGCGGCGAAGACCG | |
| | AGGCGCGTATTCGTCTGGCGATCCAACAGGGCACCCTGCCGGTGAGCAACCTGAAG | |
| | ATCACCGAAATTAGCGCGACCATCAACAGCAAAGGTCAAGTGCGTATTCCGGTTAA | |
| | GTTTGACGTGGGTCGTCAAAAAGGCACCCTGCAGATCGGTGACCGTTTCTGCGGCT | |
| | ACGATCAAAACCAGACCGCGAGCCACGCGTATAGCCTGTGGGAAGTGGTTAAAGAG | |
| | GGTCAATACCATAAAGAGCTGGGCTGCTTTGTTCGTTTCATCAGCAGCGGTGACAT | |
| | CGTGAGCATTACCGAGAACCGTGGCAACCAATTTGATCAGCTGAGCTATGAAGGTC | |
| | TGGCGTACCCGCAATATGCGGACTGGCGTAAGAAAGCGAGCAAGTTCGTGAGCCTG | |
| | TGGCAGATCACCAAGAAAAACAAGAAAAAGGAAATCGTGACCGTTGAAGCGAAAGA | |
| | GAAGTTTGACGCGATCTGCAAGTACCAGCCGCGTCTGTATAAATTCAACAAGGAGT | |
| | ACGCGTATCTGCTGCGTGATATTGTTCGTGGCAAAAGCCTGGTGGAACTGCAACAG | |
| | ATTCGTCAAGAGATCTTTCGTTTCATTGAACAGGACTGCGGTGTTACCCGTCTGGG | |
| | CAGCCTGAGCCTGAGCACCCTGGAAACCGTGAAAGCGGTTAAGGGTATCATTTACA | |
| | GCTATTTTAGCACCGCGCTGAACGCGAGCAAGAACAACCCGATCAGCGACGAACAG | |
| | CGTAAAGAGTTTGATCCGGAACTGTTCGCGCTGCTGGAAAAGCTGGAGCTGATTCG | |
| | TACCCGTAAAAAGAAACAAAAAGTGGAACGTATCGCGAACAGCCTGATTCAGACCT | |
| | GCCTGGAGAACAACATCAAGTTCATTCGTGGTGAAGGCGACCTGAGCACCACCAAC | |
| | AACGCGACCAAGAAAAAGGCGAACAGCCGTAGCATGGATTGGTTGGCGCGTGGTGT | |
| | TTTTAACAAAATCCGTCAACTGGCGCCGATGCACAACATTACCCTGTTCGGTTGCG | |
| | GCAGCCTGTACACCAGCCACCAGGACCCGCTGGTGCATCGTAACCCGGATAAAGCG | |
| | ATGAAGTGCCGTTGGGCGGCGATCCCGGTTAAGGACATTGGCGATTGGGTGCTGCG | |
| | TAAGCTGAGCCAAAACCTGCGTGCGAAAAACATCGGCACCGGCGAGTACTATCACC | |
| | AAGGTGTTAAAGAGTTCCTGAGCCATTATGAACTGCAGGACCTGGAGGAAGAGCTG | |
| | CTGAAGTGGCGTAGCGATCGTAAAAGCAACATTCCGTGCTGGGTGCTGCAGAACCG | |
| | TCTGGCGGAGAAGCTGGGCAACAAAGAAGCGGTGGTTTACATCCCGGTTCGTGGTG | |
| | GCCGTATTTATTTTGCGACCCACAAGGTGGCGACCGGTGCGGTGAGCATCGTTTTC | |
| | GACCAAAAACAAGTGTGGGTTTGCAACGCGGATCATGTTGCGGCGGCGAACATCGC | |
| | GCTGACCGTGAAGGGTATTGGCGAACAAAGCAGCGACGAAGAGAACCCGGATGGTA | |
| | GCCGTATCAAACTGCAGCTGACCAGC | |
| 922 | MSSAIKSYKSVLRPNERKNQLLKSTIQCLEDGSAFFFKMLQGLFGGITPEIVREST | Parent |
| | EQEKQQQDIALWCAVNWFRPVSQDSLTHTIASDNLVEKFEEYYGGTASDAIKQYFS | Cas12i2 |
| | ASIGESYYWNDCRQQYYDLCRELGVEVSDLTHDLEILCREKCLAVATESNQNNSII | amino acid |
| | SVLFGTGEKEDRSVKLRITKKILEAISNLKEIPKNVAPIQEIILNVAKATKETFRQ | sequence |
| | VYAGNLGAPSTLEKFIAKDGQKEFDLKKLQTDLKKVIRGKSKERDWCCQEELRSYV | |
| | EQNTIQYDLWAWGEMFNKAHTALKIKSTRNYNFAKQRLEQFKEIQSLNNLLVVKKL | |
| | NDFFDSEFFSGEETYTICVHHLGGKDLSKLYKAWEDDPADPENAIVVLCDDLKNNF | |
| | KKEPIRNILRYIFTIRQECSAQDILAAAKYNQQLDRYKSQKANPSVLGNQGFTWIN | |
| | AVILPEKAQRNDRPNSLDLRIWLYLKLRHPDGRWKKHHIPFYDTRFFQEIYAAGNS | |
| | PVDTCQFRTPRFGYHLPKLTDQTAIRVNKKHVKAAKTEARIRLAIQQGTLPVSNLK | |
| | ITEISATINSKGQVRIPVKFDVGRQKGTLQIGDRFCGYDQNQTASHAYSLWEVVKE | |
| | GQYHKELGCFVRFISSGDIVSITENRGNQFDQLSYEGLAYPQYADWRKKASKFVSL | |
| | WQITKKNKKKEIVTVEAKEKFDAICKYQPRLYKFNKEYAYLLRDIVRGKSLVELQQ | |
| | IRQEIFRFIEQDCGVTRLGSLSLSTLETVKAVKGIIYSYFSTALNASKNNPISDEQ | |
| | RKEFDPELFALLEKLELIRTRKKKQKVERIANSLIQTCLENNIKFIRGEGDLSTIN | |
| | NATKKKANSRSMDWLARGVFNKIRQLAPMHNITLFGCGSLYTSHQDPLVHRNPDKA | |
| | MKCRWAAIPVKDIGDWVLRKLSQNLRAKNIGTGEYYHQGVKEFLSHYELQDLEEEL | |
| | LKWRSDRKSNIPCWVLQNRLAEKLGNKEAVVYIPVRGGRIYFATHKVATGAVSIVF | |
| | DQKQVWVCNADHVAAANIALTVKGIGEQSSDEENPDGSRIKLQLTS | |
| 923 | MSSAIKSYKS VLRPNERKNQ LLKSTIQCLE DGSAFFFKML QGLFGGITPE | Variant |
| | IVRESTEQEK QQQDIALWCA VNWFRPVSQD SLTHTIASDN LVEKFEEYYG | Cas12i2 of |
| | GTASDAIKQY FSASIGESYY WNDCRQQYYD LCRELGVEVS DLTHDLEILC | SEQ ID NO: 3 |
| | REKCLAVATE SNQNNSIISV LFGTGEKEDR SVKLRITKKI LEAISNLKEI | of |
| | PKNVAPIQEI ILNVAKATKE TFRQVYAGNL GAPSTLEKFI AKDGQKEFDL | PCT/US2021/ |
| | KKLQTDLKKV IRGKSKERDW CCQEELRSYV EQNTIQYDLW AWGEMENKAH | 025257 |
| | TALKIKSTRN YNFAKQRLEQ FKEIQSLNNL LVVKKLNDFF DSEFFSGEET | |
| | YTICVHHLGG KDLSKLYKAW EDDPADPENA IVVLCDDLKN NFKKEPIRNI | |
| | LRYIFTIRQE CSAQDILAAA KYNQQLDRYK SQKANPSVLG NQGETWTNAV | |
| | ILPEKAQRND RPNSLDLRIW LYLKLRHPDG RWKKHHIPFY DTRFFQEIYA | |
| | AGNSPVDTCQ FRTPRFGYHL PKLTDQTAIR VNKKHVKAAK TEARIRLAIQ | |
| | QGTLPVSNLK ITEISATINS KGQVRIPVKF RVGRQKGTLQ IGDRFCGYDQ | |
| | NQTASHAYSL WEVVKEGQYH KELGCFVRFI SSGDIVSITE NRGNQFDQLS | |
| | YEGLAYPQYA DWRKKASKFV SLWQITKKNK KKEIVTVEAK EKFDAICKYQ | |
| | PRLYKFNKEY AYLLRDIVRG KSLVELQQIR QEIFRFIEQD CGVTRLGSLS | |
| | LSTLETVKAV KGIIYSYFST ALNASKNNPI SDEQRKEFDP ELFALLEKLE | |
| | LIRTRKKKQK VERIANSLIQ TCLENNIKFI RGEGDLSTIN NATKKKANSR | |
| | SMDWLARGVF NKIRQLAPMH NITLFGCGSL YTSHQDPLVH RNPDKAMKCR | |
| | WAAIPVKDIG RWVLRKLSQN LRAKNRGIGE YYHQGVKEFL SHYELQDLEE | |
| | ELLKWRSDRK SNIPCWVLQN RLAEKLGNKE AVVYIPVRGG RIYFATHKVA | |
| | TGAVSIVFDQ KQVWVCNADH VAAANIALTG KGIGEQSSDE ENPDGSRIKL | |
| | QLTS | |
| 924 | MSSAIKSYKS VLRPNERKNQ LLKSTIQCLE DGSAFFFKML QGLFGGITPE | Variant |
| | IVRESTEQEK QQQDIALWCA VNWFRPVSQD SLTHTIASDN LVEKFEEYYG | Cas12i2 of |
| | GTASDAIKQY FSASIGESYY WNDCRQQYYD LCRELGVEVS DLTHDLEILC | SEQ ID NO: 4 |
| | REKCLAVATE SNQNNSIISV LFGTGEKEDR SVKLRITKKI LEAISNLKEI | of |
| | PKNVAPIQEI ILNVAKATKE TFRQVYAGNL GAPSTLEKFI AKDGQKEFDL | PCT/US2021/ |
| | KKLQTDLKKV IRGKSKERDW CCQEELRSYV EQNTIQYDLW AWGEMENKAH | 025257 |
| | TALKIKSTRN YNFAKQRLEQ FKEIQSLNNL LVVKKLNDFF DSEFFSGEET | |
| | YTICVHHLGG KDLSKLYKAW EDDPADPENA IVVLCDDLKN NFKKEPIRNI | |
| | LRYIFTIRQE CSAQDILAAA KYNQQLDRYK SQKANPSVLG NQGETWTNAV | |
| | ILPEKAQRND RPNSLDLRIW LYLKLRHPDG RWKKHHIPFY DTRFFQEIYA | |
| | AGNSPVDTCQ FRTPRFGYHL PKLTDQTAIR VNKKHVKAAK TEARIRLAIQ | |
| | QGTLPVSNLK ITEISATINS KGQVRIPVKF RVGRQKGTLQ IGDRFCGYDQ | |
| | NQTASHAYSL WEVVKEGQYH KELGCFVRFI SSGDIVSITE NRGNQFDQLS | |
| | YEGLAYPQYA DWRKKASKFV SLWQITKKNK KKEIVTVEAK EKFDAICKYQ | |
| | PRLYKFNKEY AYLLRDIVRG KSLVELQQIR QEIFRFIEQD CGVTRLGSLS | |
| | LSTLETVKAV KGIIYSYFST ALNASKNNPI SDEQRKEFDP ELFALLEKLE | |
| | LIRTRKKKQK VERIANSLIQ TCLENNIKFI RGEGDLSTIN NATKKKANSR | |
| | SMDWLARGVF NKIRQLAPMH NITLFGCGSL YTSHQDPLVH RNPDKAMKCR | |
| | WAAIPVKDIG DWVLRKLSQN LRAKNRGIGE YYHQGVKEFL SHYELQDLEE | |
| | ELLKWRSDRK SNIPCWVLQN RLAEKLGNKE AVVYIPVRGG RIYFATHKVA | |
| | TGAVSIVFDQ KQVWVCNADH VAAANIALTG KGIGEQSSDE ENPDGSRIKL | |
| | QLTS | |
| 925 | MSSAIKSYKS VLRPNERKNQ LLKSTIQCLE DGSAFFFKML QGLFGGITPE | Variant |
| | IVRESTEQEK QQQDIALWCA VNWFRPVSQD SLTHTIASDN LVEKFEEYYG | Cas12i2 of |
| | GTASDAIKQY FSASIGESYY WNDCRQQYYD LCRELGVEVS DLTHDLEILC | SEQ ID NO: 5 |
| | REKCLAVATE SNQNNSIISV LFGTGEKEDR SVKLRITKKI LEAISNLKEI | of |
| | PKNVAPIQEI ILNVAKATKE TFRQVYAGNL GAPSTLEKFI AKDGQKEFDL | PCT/US2021/ |
| | KKLQTDLKKV IRGKSKERDW CCQEELRSYV EQNTIQYDLW AWGEMENKAH | 025257 |
| | TALKIKSTRN YNFAKQRLEQ FKEIQSLNNL LVVKKLNDFF DSEFFSGEET | |
| | YTICVHHLGG KDLSKLYKAW EDDPADPENA IVVLCDDLKN NFKKEPIRNI | |
| | LRYIFTIRQE CSAQDILAAA KYNQQLDRYK SQKANPSVLG NQGETWTNAV | |
| | ILPEKAQRND RPNSLDLRIW LYLKLRHPDG RWKKHHIPFY DTRFFQEIYA | |
| | AGNSPVDTCQ FRTPRFGYHL PKLTDQTAIR VNKKHVKAAK TEARIRLAIQ | |
| | QGTLPVSNLK ITEISATINS KGQVRIPVKF RVGRQKGTLQ IGDRFCGYDQ | |
| | NQTASHAYSL WEVVKEGQYH KELGCFVRFI SSGDIVSITE NRGNQFDQLS | |
| | YEGLAYPQYA DWRKKASKFV SLWQITKKNK KKEIVTVEAK EKFDAICKYQ | |
| | PRLYKFNKEY AYLLRDIVRG KSLVELQQIR QEIFRFIEQD CGVTRLGSLS | |
| | LSTLETVKAV KGIIYSYFST ALNASKNNPI SDEQRKEFDP ELFALLEKLE | |
| | LIRTRKKKQK VERIANSLIQ TCLENNIKFI RGEGDLSTIN NATKKKANSR | |
| | SMDWLARGVF NKIRQLAPMH NITLFGCGSL YTSHQDPLVH RNPDKAMKCR | |
| | WAAIPVKDIG DWVLRKLSQN LRAKNRGTGE YYHQGVKEFL SHYELQDLEE | |
| | ELLKWRSDRK SNIPCWVLQN RLAEKLGNKE AVVYIPVRGG RIYFATHKVA | |
| | TGAVSIVFDQ KQVWVCNADH VAAANIALTG KGIGEQSSDE ENPDGGRIKL | |
| | QLTS | |
| 926 | MSSAIKSYKS VLRPNERKNQ LLKSTIQCLE DGSAFFFKML QGLFGGITPE | Variant |
| | IVRESTEQEK QQQDIALWCA VNWFRPVSQD SLTHTIASDN LVEKFEEYYG | Cas12i2 of |
| | GTASDAIKQY FSASIGESYY WNDCRQQYYD LCRELGVEVS DLTHDLEILC | SEQ ID NO: |
| | REKCLAVATE SNQNNSIISV LFGTGEKEDR SVKLRITKKI LEAISNLKEI | 495 of |
| | PKNVAPIQEI ILNVAKATKE TFRQVYAGNL GAPSTLEKFI AKDGQKEFDL | PCT/US2021/ |
| | KKLQTDLKKV IRGKSKERDW CCQEELRSYV EQNTIQYDLW AWGEMENKAH | 025257 |
| | TALKIKSTRN YNFAKQRLEQ FKEIQSLNNL LVVKKLNDFF DSEFFSGEET | |
| | YTICVHHLGG KDLSKLYKAW EDDPADPENA IVVLCDDLKN NFKKEPIRNI | |
| | LRYIFTIRQE CSAQDILAAA KYNQQLDRYK SQKANPSVLG NQGFTWTNAV | |
| | ILPEKAQRND RPNSLDLRIW LYLKLRHPDG RWKKHHIPFY DTRFFQEIYA | |
| | AGNSPVDTCQ FRTPRFGYHL PKLTDQTAIR VNKKHVKAAK TEARIRLAIQ | |
| | QGTLPVSNLK ITEISATINS KGQVRIPVKF RVGRQKGTLQ IGDRFCGYDQ | |
| | NQTASHAYSL WEVVKEGQYH KELRCRVRFI SSGDIVSITE NRGNQFDQLS | |
| | YEGLAYPQYA DWRKKASKFV SLWQITKKNK KKEIVTVEAK EKFDAICKYQ | |
| | PRLYKENKEY AYLLRDIVRG KSLVELQQIR QEIFRFIEQD CGVTRLGSLS | |
| | LSTLETVKAV KGIIYSYFST ALNASKNNPI SDEQRKEFDP ELFALLEKLE | |
| | LIRTRKKKQK VERIANSLIQ TCLENNIKFI RGEGDLSTIN NATKKKANSR | |
| | SMDWLARGVF NKIRQLAPMH NITLFGCGSL YTSHQDPLVH RNPDKAMKCR | |
| | WAAIPVKDIG DWVLRKLSQN LRAKNRGIGE YYHQGVKEFL SHYELQDLEE | |
| | ELLKWRSDRK SNIPCWVLQN RLAEKLGNKE AVVYIPVRGG RIYFATHKVA | |
| | TGAVSIVFDQ KQVWVCNADH VAAANIALTG KGIGRQSSDE ENPDGGRIKL | |
| | QLTS | |
| 927 | MSSAIKSYKS VLRPNERKNQ LLKSTIQCLE DGSAFFFKML QGLFGGITPE | Variant |
| | IVRESTEQEK QQQDIALWCA VNWFRPVSQD SLTHTIASDN LVEKFEEYYG | Cas12i2 of |
| | GTASDAIKQY FSASIGESYY WNDCRQQYYD LCRELGVEVS DLTHDLEILC | SEQ ID NO: |
| | REKCLAVATE SNQNNSIISV LFGTGEKEDR SVKLRITKKI LEAISNLKEI | 496 of |
| | PKNVAPIQEI ILNVAKATKE TFRQVYAGNL GAPSTLEKFI AKDGQKEFDL | PCT/US2021/ |
| | KKLQTDLKKV IRGKSKERDW CCQEELRSYV EQNTIQYDLW AWGEMENKAH | 025257 |
| | TALKIKSTRN YNFAKQRLEQ FKEIQSLNNL LVVKKLNDFF DSEFFSGEET | |
| | YTICVHHLGG KDLSKLYKAW EDDPADPENA IVVLCDDLKN NFKKEPIRNI | |
| | LRYIFTIRQE CSAQDILAAA KYNQQLDRYK SQKANPSVLG NQGETWTNAV | |
| | ILPEKAQRND RPNSLDLRIW LYLKLRHPDG RWKKHHIPFY DTRFFQEIYA | |
| | AGNSPVDTCQ FRTPRFGYHL PKLTDQTAIR VNKKHVKAAK TEARIRLAIQ | |
| | QGTLPVSNLK ITEISATINS KGQVRIPVKF RVGRQKGTLQ IGDRFCGYDQ | |
| | NQTASHAYSL WEVVKEGQYH KELRCRVRFI SSGDIVSITE NRGNQFDQLS | |
| | YEGLAYPQYA DWRKKASKFV SLWQITKKNK KKEIVTVEAK EKFDAICKYQ | |
| | PRLYKENKEY AYLLRDIVRG KSLVELQQIR QEIFRFIEQD CGVTRLGSLS | |
| | LSTLETVKAV KGIIYSYFST ALNASKNNPI SDEQRKEFDP ELFALLEKLE | |
| | LIRTRKKKQK VERIANSLIQ TCLENNIKFI RGEGDLSTIN NATKKKANSR | |
| | SMDWLARGVF NKIRQLATMH NITLFGCGSL YTSHQDPLVH RNPDKAMKCR | |
| | WAAIPVKDIG DWVLRKLSQN LRAKNRGTGE YYHQGVKEFL SHYELQDLEE | |
| | ELLKWRSDRK SNIPCWVLQN RLAEKLGNKE AVVYIPVRGG RIYFATHKVA | |
| | TGAVSIVFDQ KQVWVCNADH VAAANIALTG KGIGRQSSDE ENPDGGRIKL | |
| | QLTS | |
| 955 | ATGGCTTCCATCTCTAGGCCATACGGCACCAAGCTGCGACCGGACGCACGGAAGAA | Nucleotide |
| | GGAGATGCTCGATAAGTTCTTTAATACACTGACTAAGGGTCAGCGCGTGTTCGCAG | sequence |
| | ACCTGGCCCTGTGCATCTATGGCTCCCTGACCCTGGAGATGGCCAAGTCTCTGGAG | encoding |
| | CCAGAAAGTGATTCAGAACTGGTGTGCGCTATTGGGTGGTTTCGGCTGGTGGACAA | parent |
| | GACCATCTGGTCCAAGGATGGCATCAAGCAGGAGAATCTGGTGAAACAGTACGAAG | Cas12i4 |
| | CCTATTCCGGAAAGGAGGCTTCTGAAGTGGTCAAAACATACCTGAACAGCCCCAGC | |
| | TCCGACAAGTACGTGTGGATCGATTGCAGGCAGAAATTCCTGAGGTTTCAGCGCGA | |
| | GCTCGGCACTCGCAACCTGTCCGAGGACTTCGAATGTATGCTCTTTGAACAGTACA | |
| | TTAGACTGACCAAGGGCGAGATCGAAGGGTATGCCGCTATTTCAAATATGTTCGGA | |
| | AACGGCGAGAAGGAAGACCGGAGCAAGAAAAGAATGTACGCTACACGGATGAAAGA | |
| | TTGGCTGGAGGCAAACGAAAATATCACTTGGGAGCAGTATAGAGAGGCCCTGAAGA | |
| | ACCAGCTGAATGCTAAAAACCTGGAGCAGGTTGTGGCCAATTACAAGGGGAACGCT | |
| | GGCGGGGCAGACCCCTTCTTTAAGTATAGCTTCTCCAAAGAGGGAATGGTGAGCAA | |
| | GAAAGAACATGCACAGCAGCTCGACAAGTTCAAAACCGTCCTGAAGAACAAAGCCC | |
| | GGGACCTGAATTTTCCAAACAAGGAGAAGCTGAAGCAGTACCTGGAGGCCGAAATC | |
| | GGCATTCCGGTCGACGCTAACGTGTACTCCCAGATGTTCTCTAACGGGGTGAGTGA | |
| | GGTCCAGCCTAAGACCACACGGAATATGTCTTTTAGTAACGAGAAACTGGATCTGC | |
| | TCACTGAACTGAAGGACCTGAACAAGGGCGATGGGTTCGAGTACGCCAGAGAAGTG | |
| | CTGAACGGGTTCTTTGACTCCGAGCTCCACACTACCGAGGATAAGTTTAATATCAC | |
| | CTCTAGGTACCTGGGAGGCGACAAATCAAACCGCCTGAGCAAACTCTATAAGATCT | |
| | GGAAGAAAGAGGGTGTGGACTGCGAGGAAGGCATTCAGCAGTTCTGTGAAGCCGTC | |
| | AAAGATAAGATGGGCCAGATCCCCATTCGAAATGTGCTGAAGTACCTGTGGCAGTT | |
| | CCGGGAGACAGTCAGTGCCGAGGATTTTGAAGCAGCCGCTAAGGCTAACCATCTGG | |
| | AGGAAAAGATCAGCCGGGTGAAAGCCCACCCAATCGTGATTAGCAATAGGTACTGG | |
| | GCTTTTGGGACTTCCGCACTGGTGGGAAACATTATGCCCGCAGACAAGAGGCATCA | |
| | GGGAGAGTATGCCGGTCAGAATTTCAAAATGTGGCTGGAGGCTGAACTGCACTACG | |
| | ATGGCAAGAAAGCAAAGCACCATCTGCCTTTTTATAACGCCCGCTTCTTTGAGGAA | |
| | GTGTACTGCTATCACCCCTCTGTCGCCGAGATCACTCCTTTCAAAACCAAGCAGTT | |
| | TGGCTGTGAAATCGGGAAGGACATTCCAGATTACGTGAGCGTCGCTCTGAAGGACA | |
| | ATCCGTATAAGAAAGCAACCAAACGAATCCTGCGTGCAATCTACAATCCCGTCGCC | |
| | AACACAACTGGCGTTGATAAGACCACAAACTGCAGCTTCATGATCAAACGCGAGAA | |
| | TGACGAATATAAGCTGGTCATCAACCGAAAAATTTCCGTGGATCGGCCTAAGAGAA | |
| | TCGAAGTGGGCAGGACAATTATGGGGTACGACCGCAATCAGACAGCTAGCGATACT | |
| | TATTGGATTGGCCGGCTGGTGCCACCTGGAACCCGGGGCGCATACCGCATCGGAGA | |
| | GTGGAGCGTCCAGTATATTAAGTCCGGGCCTGTCCTGTCTAGTACTCAGGGAGTTA | |
| | ACAATTCCACTACCGACCAGCTGGTGTACAACGGCATGCCATCAAGCTCCGAGCGG | |
| | TTCAAGGCCTGGAAGAAAGCCAGAATGGCTTTTATCCGAAAACTCATTCGTCAGCT | |
| | GAATGACGAGGGACTGGAATCTAAGGGTCAGGATTATATCCCCGAGAACCCTTCTA | |
| | GTTTCGATGTGCGGGGCGAAACCCTGTACGTCTTTAACAGTAATTATCTGAAGGCC | |
| | CTGGTGAGCAAACACAGAAAGGCCAAGAAACCTGTTGAGGGGATCCTGGACGAGAT | |
| | TGAAGCCTGGACATCTAAAGACAAGGATTCATGCAGCCTGATGCGGCTGAGCAGCC | |
| | TGAGCGATGCTTCCATGCAGGGAATCGCCAGCCTGAAGAGTCTGATTAACAGCTAC | |
| | TTCAACAAGAATGGCTGTAAAACCATCGAGGACAAAGAAAAGTTTAATCCCGTGCT | |
| | GTATGCCAAGCTGGTTGAGGTGGAACAGCGGAGAACAAACAAGCGGTCTGAGAAAG | |
| | TGGGAAGAATCGCAGGTAGTCTGGAGCAGCTGGCCCTGCTGAACGGGGTTGAGGTG | |
| | GTCATCGGCGAAGCTGACCTGGGGGAGGTCGAAAAAGGAAAGAGTAAGAAACAGAA | |
| | TTCACGGAACATGGATTGGTGCGCAAAGCAGGTGGCACAGCGGCTGGAGTACAAAC | |
| | TGGCCTTCCATGGAATCGGTTACTTTGGAGTGAACCCCATGTATACCAGCCACCAG | |
| | GACCCTTTCGAACATAGGCGCGTGGCTGATCACATCGTCATGCGAGCACGTTTTGA | |
| | GGAAGTCAACGTGGAGAACATTGCCGAATGGCACGTGCGAAATTTCTCAAACTACC | |
| | TGCGTGCAGACAGCGGCACTGGGCTGTACTATAAGCAGGCCACCATGGACTTCCTG | |
| | AAACATTACGGTCTGGAGGAACACGCTGAGGGCCTGGAAAATAAGAAAATCAAGTT | |
| | CTATGACTTTAGAAAGATCCTGGAGGATAAAAACCTGACAAGCGTGATCATTCCAA | |
| | AGAGGGGGGGGCGCATCTACATGGCCACCAACCCAGTGACATCCGACTCTACCCCG | |
| | ATTACATACGCCGGCAAGACTTATAATAGGTGTAACGCTGATGAGGTGGCAGCCGC | |
| | TAATATCGTTATTTCTGTGCTGGCTCCCCGCAGTAAGAAAAACGAGGAACAGGACG | |
| | ATATCCCTCTGATTACCAAGAAAGCCGAGAGTAAGTCACCACCGAAAGACCGGAAG | |
| | AGATCAAAAACAAGCCAGCTGCCTCAGAAA | |
| 956 | MASISRPYGTKLRPDARKKEMLDKFFNTLTKGQRVFADLALCIYGSLTLEMAKSLE | Parent |
| | PESDSELVCAIGWFRLVDKTIWSKDGIKQENLVKQYEAYSGKEASEVVKTYLNSPS | Cas12i4 |
| | SDKYVWIDCRQKFLRFQRELGTRNLSEDFECMLFEQYIRLTKGEIEGYAAISNMFG | amino acid |
| | NGEKEDRSKKRMYATRMKDWLEANENITWEQYREALKNQLNAKNLEQVVANYKGNA | sequence |
| | GGADPFFKYSFSKEGMVSKKEHAQQLDKFKTVLKNKARDLNFPNKEKLKQYLEAEI | |
| | GIPVDANVYSQMFSNGVSEVQPKTTRNMSFSNEKLDLLTELKDLNKGDGFEYAREV | |
| | LNGFFDSELHTTEDKFNITSRYLGGDKSNRLSKLYKIWKKEGVDCEEGIQQFCEAV | |
| | KDKMGQIPIRNVLKYLWQFRETVSAEDFEAAAKANHLEEKISRVKAHPIVISNRYW | |
| | AFGTSALVGNIMPADKRHQGEYAGQNFKMWLEAELHYDGKKAKHHLPFYNARFFEE | |
| | VYCYHPSVAEITPFKTKQFGCEIGKDIPDYVSVALKDNPYKKATKRILRAIYNPVA | |
| | NTTGVDKTTNCSFMIKRENDEYKLVINRKISVDRPKRIEVGRTIMGYDRNQTASDT | |
| | YWIGRLVPPGTRGAYRIGEWSVQYIKSGPVLSSTQGVNNSTTDQLVYNGMPSSSER | |
| | FKAWKKARMAFIRKLIRQLNDEGLESKGQDYIPENPSSFDVRGETLYVENSNYLKA | |
| | LVSKHRKAKKPVEGILDEIEAWTSKDKDSCSLMRLSSLSDASMQGIASLKSLINSY | |
| | FNKNGCKTIEDKEKFNPVLYAKLVEVEQRRINKRSEKVGRIAGSLEQLALLNGVEV | |
| | VIGEADLGEVEKGKSKKQNSRNMDWCAKQVAQRLEYKLAFHGIGYFGVNPMYTSHQ | |
| | DPFEHRRVADHIVMRARFEEVNVENIAEWHVRNFSNYLRADSGTGLYYKQATMDFL | |
| | KHYGLEEHAEGLENKKIKFYDFRKILEDKNLISVIIPKRGGRIYMATNPVTSDSTP | |
| | ITYAGKTYNRCNADEVAAANIVISVLAPRSKKNEEQDDIPLITKKAESKSPPKDRK | |
| | RSKTSQLPQK | |
| 957 | MASISRPYGT KLRPDARKKE MLDKFFNTLT KGQRVFADLA LCIYGSLTLE | Variant |
| | MAKSLEPESD SELVCAIGWF RLVDKTIWSK DGIKQENLVK QYEAYSGKEA | Cas 12i4 A |
| | SEVVKTYLNS PSSDKYVWID CRQKFLRFQR ELGTRNLSED FECMLFEQYI | |
| | RLTKGEIEGY AAISNMFGNG EKEDRSKKRM YATRMKDWLE ANENITWEQY | |
| | REALKNQLNA KNLEQVVANY KGNAGGADPF FKYSFSKEGM VSKKEHAQQL | |
| | DKFKTVLKNK ARDLNFPNKE KLKQYLEAEI GIPVDANVYS QMFSNGVSEV | |
| | QPKTTRNMSF SNEKLDLLTE LKDLNKGDGF EYAREVLNGF FDSELHTTED | |
| | KFNITSRYLG GDKSNRLSKL YKIWKKEGVD CEEGIQQFCE AVKDKMGQIP | |
| | IRNVLKYLWQ FRETVSAEDF EAAAKANHLE EKISRVKAHP IVISNRYWAF | |
| | GTSALVGNIM PADKRHQGEY AGQNFKMWLE AELHYDGKKA KHHLPFYNAR | |
| | FFEEVYCYHP SVAEITPFKT KQFGCEIGKD IPDYVSVALK DNPYKKATKR | |
| | ILRAIYNPVA NTTGVDKITN CSFMIKREND EYKLVINRKI SRDRPKRIEV | |
| | GRTIMGYDRN QTASDTYWIG RLVPPGTRGA YRIGEWSVQY IKSGPVLSST | |
| | QGVNNSTTDQ LVYNGMPSSS ERFKAWKKAR MAFIRKLIRQ LNDEGLESKG | |
| | QDYIPENPSS FDVRGETLYV FNSNYLKALV SKHRKAKKPV EGILDEIEAW | |
| | TSKDKDSCSL MRLSSLSDAS MQGIASLKSL INSYFNKNGC KTIEDKEKEN | |
| | PVLYAKLVEV EQRRTNKRSE KVGRIAGSLE QLALLNGVEV VIGEADLGEV | |
| | EKGKSKKQNS RNMDWCAKQV AQRLEYKLAF HGIGYFGVNP MYTSHQDPFE | |
| | HRRVADHIVM RARFEEVNVE NIAEWHVRNF SNYLRADSGT GLYYKQATMD | |
| | FLKHYGLEEH AEGLENKKIK FYDERKILED KNLISVIIPK RGGRIYMAIN | |
| | PVTSDSTPIT YAGKTYNRCN ADEVAAANIV ISVLAPRSKK NREQDDIPLI | |
| | TKKAESKSPP KDRKRSKTSQ LPQK | |
| 958 | MASISRPYGT KLRPDARKKE MLDKFFNTLT KGQRVFADLA LCIYGSLTLE | Variant |
| | MAKSLEPESD SELVCAIGWF RLVDKTIWSK DGIKQENLVK QYEAYSGKEA | Cas12i4 B |
| | SEVVKTYLNS PSSDKYVWID CRQKFLRFQR ELGTRNLSED FECMLFEQYI | |
| | RLTKGEIEGY AAISNMFGNG EKEDRSKKRM YATRMKDWLE ANENITWEQY | |
| | REALKNQLNA KNLEQVVANY KGNAGGADPF FKYSFSKEGM VSKKEHAQQL | |
| | DKFKTVLKNK ARDLNFPNKE KLKQYLEAEI GIPVDANVYS QMFSNGVSEV | |
| | QPKTTRNMSF SNEKLDLLTE LKDLNKGDGF EYAREVLNGF FDSELHTTED | |
| | KFNITSRYLG GDKSNRLSKL YKIWKKEGVD CEEGIQQFCE AVKDKMGQIP | |
| | IRNVLKYLWQ FRETVSAEDF EAAAKANHLE EKISRVKAHP IVISNRYWAF | |
| | GTSALVGNIM PADKRHQGEY AGQNFKMWLR AELHYDGKKA KHHLPFYNAR | |
| | FFEEVYCYHP SVAEITPFKT KQFGCEIGKD IPDYVSVALK DNPYKKATKR | |
| | ILRAIYNPVA NTTRVDKTIN CSFMIKREND EYKLVINRKI SRDRPKRIEV | |
| | GRTIMGYDRN QTASDTYWIG RLVPPGTRGA YRIGEWSVQY IKSGPVLSST | |
| | QGVNNSTTDQ LVYNGMPSSS ERFKAWKKAR MAFIRKLIRQ LNDEGLESKG | |
| | QDYIPENPSS FDVRGETLYV FNSNYLKALV SKHRKAKKPV EGILDEIEAW | |
| | TSKDKDSCSL MRLSSLSDAS MQGIASLKSL INSYFNKNGC KTIEDKEKEN | |
| | PVLYAKLVEV EQRRTNKRSE KVGRIAGSLE QLALLNGVEV VIGEADLGEV | |
| | EKGKSKKQNS RNMDWCAKQV AQRLEYKLAF HGIGYFGVNP MYTSHQDPFE | |
| | HRRVADHIVM RARFEEVNVE NIAEWHVRNF SNYLRADSGT GLYYKQATMD | |
| | FLKHYGLEEH AEGLENKKIK FYDFRKILED KNLISVIIPK RGGRIYMATN | |
| | PVTSDSTPIT YAGKTYNRCN ADEVAAANIV ISVLAPRSKK NREQDDIPLI | |
| | TKKAESKSPP KDRKRSKISQ LPQK | |
| 928 | AACGAACTCCATCTGGGATAGCAATAACCTGTGAAAATGCTCCCCCGGCTAATTTG | HAQ1 |
| | TATCAATGATTATGAACAACATGCTAAATCAGTACTTCCAAAGTCTATATATGACT | |
| | ATTACAGGTCTGGGGCAAATGATGAAGAAACTTTGGCTGATAATATTGCAGCATTT | |
| | TCCAGGTAAGAAAATTTATTTTTTAAAATCATGTTTTAAAATTACACAAAGACCGT | |
| | ACCAAAATAAGATCTCCTAGTTTTACGTTGGTGGTGTGTAATTATTTGTTCAGATT | |
| | TGTGCTTAGTAGAGAGGGAAAAGTTCTTGGGGCTGTAAGAAATCTTGGGCCTTTAA | |
| | ATTGTTAAAAAATATTCCAAGCCTGTGAATCTTGAGGAACTGACTGCAAAAGCCAA | |
| | ACCTATGTTACTTCACTTGGAAATATGACAACAATTAATTTAACTACATGTAAAAA | |
| | TAGCGATAAATTCGGATGACTTTTCTTTTTCTTAGTATGACAGTAAATGCTTATGT | |
| | TCATGGTGTAGGAAACAGCATTAAATGCCAGATAACCATCTTATCCGGATGAACCA | |
| | GACTGGATTGTTGGCTCAAATGTTTTCTTCCTGCTGGCTTTTCGTGTTATCATTCA | |
| | TTTTGATTACTGTTGTCTAAACTTTCACTTTAGATTTCAATTTGTCTATGCAGCAT | |
| | TAATCTTTCAACTTTGCTGTTTCATCTCTCCTTCAAAGCACTTCATCTCTCTTCCC | |
| | AAATTAGTTTTCCTTTGACTTTCATATTTCAAAGCACAAGATGGTGGGTGACATGG | |
| | TTTATGTTTTCTGTTTGTAATAAAAACAAGAAATAAAATCATTTCAAAGGGTTTTT | |
| | TTTTATAGCAGTTACAAAAATGGTTTATTTGCTGGAGCAAGAGAGGAGTGCCTTCA | |
| | CTACACTACACTCAGTCTCATCCATCTAACATTATGGCTGTTAGTAAAGGCAATCG | |
| | GTATTGTGGGTACTCATTGATGGTGATAAAGACAAAAAGGCAGAAAATATGCAGGG | |
| | GGAGAGAATTAGCCTTCCTCCCTGATTTCTTCTTTAGTCTACAACAAAATCACTCA | |
| | AAATCAGTTTTCCATATTTAAATTAGGAGAAATAAAATTATCCTGGCCAAGGTGGT | |
| | CTCTGGTAGGCAGCACTGATTCACCCACAAATCCATGTAGAAGACTGAAAATGGCA | |
| | ATGGGGTGAAGGATACGGCCTCTCCCCAACCCTTTCAAGCCTTGACTTTGTCTCAG | |
| | GTTTTGCCTGGAACCCAAATGAGCTCAACAAATGCCAGGGAAGTCATGGGAAGGGA | |
| | AGTTGACTGAGAGTAGAGGGGCTTAAAATTCTGCATCATTATTTACTATTTTGGAC | |
| | TCATTTAAAAGTTTCTGCTCTTGGAAGATGCCCCTTCTTGGGCCGATATTAACTTT | |
| | GTCCACCAAAATTTGCCTATGAGTGGTCTCTTGAAAACACTTTAACCCAAATAGGT | |
| | TATTACAACCAAGGAAATTTCAGACCCTTGACAGATTTATAGAGTTAGTGTCTCAG | |
| | CATTGCTAGACCTCCAATGCTCAAGTGATTATTTATTTCATTTGTATACAGCTTTC | |
| | CTTACTTCTTAATTCCCTTTGTCGCATGCTAGCTAATTAACTAGAGCTAATTAGGA | |
| | GTCTCCATGAGCTACACTGTGTACTACATGCTGAGGACAAAGCAGTGAGCCAGACA | |
| | AAGTTCCTGTCCCTAGGAACTTACATTCCCCTGGATGCATATCAGCCTCCATAATG | |
| | CTGTTGGGTTGAATTGATGCAAAATGGGCCCAAAATAGTTGGCCAAGTGGAGGTCT | |
| | CAGAGAGGATGCAAAGGGGCGCCCCAAAGCAGATGGATCACCTATGCAACCCTTTA | |
| | AAATGTAGAAACTTTGGGAGACATAGAAGGCTTGGTGACTTCTAAGTTATGAACTG | |
| | GAAAAGTGCCTCATGCCTTATGTGAATTACATGGTATTCAAGTGAGTATTCCCATC | |
| | CTATGTGTGTACCGAGTAACTTAGGGATAGGACACAGATAATGAAAATGAATTTGC | |
| | AGTGTCACCTTTTCCATGAACCTTGATCATTCTCTTTTGTTCAGCTTTAAATTAAA | |
| | AAAAAAAATCAATCAACTTTCTTTGGAGGACAGCTGATGCTATTTTATTATCAACT | |
| | AGTTGAGTTTTTATTGCAATACATTTTGCAATGTGTCCTCTTTTGCTGTATGACTC | |
| | GCTAGGTGAACCTTGATTCCTCACACTGCATCATGTAGCTGGTCACGTGAAACTAA | |
| | GAATAGAAATTCTGCCAGGGTTGTGGAGACTTTGGGTTGATGGCATGAAGGAAATC | |
| | AACCTGAAATTTCACATTCTGATTCTAATGAAAAGTGCAAAACAATCAAACCTCAG | |
| | ATAACCCATTGTGATACAAAGCCAGAGTATTTCAAACACATTTATGAAATTTATAC | |
| | ACCTCCCCATCTCGCAAGTACAACAAAAGGTCATTCACCGTGACAGCTTTTATTTC | |
| | TCTGTACTCAGCTCTGATAATCACATTTTGGAGTTCTGGGGACATGGACCACTCAT | |
| | GTGACCCAGCAGTTGCTTGGAGATATTTTTGGGTAAGACTTCAGACTAATATTACT | |
| | GTGGCAGTAGAAAAAAATGTTTAAAAGGACAAGTAAATGGAACCACCCAGAACAAA | |
| | ATTTCTTACGGTGGTTATAACAAAACAGGGTAAATGTCAACTTGCTACATTTTGCA | |
| | TGGCTGGAATTGATTGGGATTAATTCAACGAAGAACAGTAATTTGTTTCTCTTACA | |
| | CATTTATTCAAAGTAGCCTTCTCAACTATGGTCTTCACGTTGTTGTAGCTTTTTTT | |
| | TCTGAAATTATCAATGATGGAAGATGATTAAACAATTTCGACACTTAGAAGCCCTC | |
| | ATGATTTCAGAAAAGGAAACTCTTTTCTGCTGCGTTACCTATTGAGACTGAAGATG | |
| | GCATCATTTTCTTTTAAATAACAGATGGGTAAAAGTGATGTCATTCTTTCACTTTA | |
| | ATATTTGAGAAGTGATATGAAGTTACCAGTGACATTGTGTTCTCATAGGCATAAAT | |
| | GTCACAAAATAATTTATCTAGTATCCACAATAGGTGAATAAGGTGTTTTTGCTTTA | |
| | TATATTTTAACTGTTTAGAGTAAAAAATTAATGTGGAGAAAATTGGAATGCAGTAT | |
| | TATAGGATTACACAACTTACAAAACATGAATCCACTATGTCCAGTTAGTGTGATTC | |
| | AGAAACAGCATGCAGTTATAAAGCTGGGTGAGGCATGGGTGTCTTCCTTCAACAGG | |
| | GCAGCTACTTTGTGAGGAGTGTATATATCATTTGATTTTTTTATAAGTTAAATTTG | |
| | AGGCCCCTGTTAGATGTGAGGGTGGGCCAAAATTCCTGTGAACAGATTCTCCCCGT | |
| | TACCCCGCTTCCTTTACTCTGGCATCTCATTTTCTATCCTTTGAAAACGGTTTATT | |
| | ATTCAATTGGTTCAACTGTTTGCCAGTTGAACCAATTCTTTTTCCAAAGTGGAGGC | |
| | CCAGGAAAGCACAGTCCGAGAATATAGTGAGGTGCTATTTTATGTATGATTGTGGG | |
| | AAATTTACTTAAATTTGGAGTGGGGTTGGGCAAGGCTTGGAAAGCTAGTGAGCTAT | |
| | CTGACATAGTTGTTACTACTATTTGAAAAATATCAAAACATGGAGGACTCTTTAGA | |
| | TAACATGCCTGTTCCCATTCCATTGATTTTATCTAATTTTACGTAGCAATTACGTT | |
| | TTGTGCATTGGTTGACAAGCCTCTGTATTATCCTCAGAACAGAAAATACTGTTTAA | |
| | GGGAAATTAAGAGCCCGCAGTTACTAAAGTGACTGCGCCACCAAGTGGACAAGTGT | |
| | AAAGCCACTGTCTGGAGATGGAAGGATTCAGCTTTGCTTTATAAATGGGAATTTGA | |
| | CCTTTAAAAATGTCCCTTTTGGCACGCACGCGCGCGCGCGCGCGCGAACACACACA | |
| | CACACACACACACACACACACACACACACACACACGGCTGCTGCCCTGCAGATTTG | |
| | CTTGTTCTTGTCATAAAGCTTTCATTGTTTCTCTAGCTCTAAGTAAATATTAATGC | |
| | CTTCCAAGGCTGGCATGCCAATGGCTGCTATTAAGATCGTTTTCTCTCATTCTAAT | |
| | AACACACTTAGAGATGATTGGTAATAAAAACTCTCTTCAAGGCTTCTGCTTCTCCC | |
| | CCTTCAAAATGGAGATCAAAGAATCATGCTGTGAGGGTCCGTCAAGAAGAAAAGAC | |
| | TTTCAGCAACAGAGCATGTGGTGTGGCATAAAATAATGACAATTATAATGTTCAAA | |
| | GGAATAGCATAGAAATCACACAGTAAAACTTCTTTATTATGCTTTTCAGGGACTGG | |
| | ATGTTTTTACTTTATTATGTGAGGAAGGGTTAGATTACAGACCCTTAGCTATTCCA | |
| | CAAAGCAATAGAAGGCAGAATTTCTTCTTCCGCTACAGGAAGCACGCTTCGATTAA | |
| | GGGCTTTTTCTTTTTCTTCTTTTTTTTTCTTTAAGTTACTGCATTACTATATCATA | |
| | CTTCACTATATTTACTAAAAAGTCATGCTGTTTCTGGAAGTAGAGTTACATCTAGG | |
| | AAATACTAGGTGAATGCTGGTTAGATATGCATGTGTGCCTAAACAACACGTTTATT | |
| | ATACTCATGCATACTAGAAATAGGGCTGTATTTTCTTCAATTTTAATCAGTACTAA | |
| | TGAGAATAATAAATCAAAACAAATAGGAGAGATATATTTTGCCAGGAGGAAAGAGA | |
| | ACTAGTTCTTCTGTAAATTTTACTGGTGAATTTTTGGTTGCTGGTTTATTGGTAAT | |
| | TTTCATTCCAACACAGAAGAATCACAGAAACATTCATTTAAAATAATTTTCCGGAG | |
| | TCAAAAACTTTTTAACACCCAAATTTCAGTTTTTGTCAAATAACATTTTTGAGAAA | |
| | AGTGTTAAATTAAACTAATAAAAAACCTTCCCTCATCATTAGACTTTAATGAATAT | |
| | GGCATATAACTAAATAATTTTGAAGAAACCAAATTATAATTTTAAAAGTAATTGCC | |
| | TGAAGCTGCTGTTTATCACATAAAAAGAAGACAAACTAGACATAGCATATCTTCTT | |
| | AAACTCTAATCTAAACTCTATGCATTTGTATACCATCTTGATTTTCAAGATTGGGG | |
| | AAGTGAAACGAAAACTATGTTCACACAAGAACCTGTACGTGAATGTTTGTAGTGGC | |
| | TTTATTTAGAATTTCCCCCCAAACTGTAAGTATTCAAAATGTCTTTTAGCTTGGGA | |
| | ATGACTGGACAAATGATAGTACCCCTGTATGATGGAATATTATTCATCAACCAAAA | |
| | GGAACAAACTATTGACACGTACAACAACATGAGAAAATCTCTAATGCGTTATGTTA | |
| | AGTGAAAGAAGCCAAACTCAAAAGGCTACATACTGAATGATTTTGTTTACATGATA | |
| | TTCTTGCAAAGCAAAATTATCAGGACAAAGAAAAAATGCATCAGTGGTTGTCAGGG | |
| | GATTGAACTGGGGAGAGTTTCTCTGCAAAAGAAAATGGGGACTTTTTTGGGAATGA | |
| | TTGAACTTTTTCTAGATCTTGATTGTCATGGCAGTTACACCACTGTATGCATTTGT | |
| | CAAAATTCACAAAACTGCAGACTAAAATGAGTGAATACTATTATGTATTAGTTATA | |
| | CTTTAATAAATAATTGCTTGGGAAATTCATTATCCTCTAATTGTTAACTTTCTAAC | |
| | CAAACAAACAGTAAAATTGCCTCTTTTCCATTAGCTTTATGAAGTCATTTGCTTGT | |
| | TTGGAAAAAATCCAATTATATTTTTTCTTTTAACTAAAATGTAATGTCAAAGTTTT | |
| | GGTTATGATTCTGAAACTCTAAAGCCTTTTATTTTATTTTATTTTTTAATTCTAGA | |
| | TGGAAGCTGTATCCAAGGATGCTCCGGAATGTTGCTGAAACAGATCTGTCGACTTC | |
| | TGTTTTAGGACAGAGGGTCAGCATGCCAATATGTGTGGGGGCTACGGCCATGCAGC | |
| | GCATGGCTCATGTGGACGGCGAGCTTGCCACTGTGAGAGGTAGGAGGAAGATTGTC | |
| | ACCACAGGGACAGAAGGAGGCTAACGTTTATCGACCTCCTTCTCTGAATGCACCAA | |
| | GCAAATATGTTCCTTGATGTTTTTACACTCAGAAACATTAAGCTCATGGACTCTAT | |
| | CATCAAAATACTTGTTCTTGCATGTCCTGCTCCTCTTCTTTCCAGCTGTGTGACTG | |
| | GGCAAGATATCCTCTCTCTGCATTGGTTTCCTTGGCTGTAAAATAGGGACAAAAAT | |
| | TGTACCTGCCTCATTGGGTTATGGTGAGAATTGAATGAGTTCAGGTATACAAAGTT | |
| | CATGGCAGAGAGTAGGGGCTCAGTAACTGTTGGTTATATTATGGGTATTAATAGTA | |
| | CTGTCTCAGGAAATGGATCTCTGACAGGTAGACTTGCCCAAAGTCACAGCTAGGTA | |
| | GTTACAGAATTGGAATTCAGCCCTGTGGCTACCTTATCTCAAAACCCTCCTGCTTC | |
| | CCCCAAACCAAAGTGGTTCTCACAGCCAAATTGCAAATGGAGCAACGTGGTTGGTT | |
| | GTGTTTTCTTCCGTGGTTTTGGGTCATGATTCTTTTTTATGGATGAGTTATATTCC | |
| | CAATAGAGCAGTTCCAGCTGTCTTAGGAGGGAGTGATGAGAAAATCAAATATGATG | |
| | TAAAGAAATCTCTTATTAGGGCTAATTTATTAACTTTCCAGTTCTCTAGCAACTGT | |
| | GAACATTTGAAAGGCTGTGCAGAGTAAAAAATCTCCCCAAATTGTGCTCCAGAAAC | |
| | TAATATAAAAGTTGGAAATGAATTATTTTGATGCTAAGCAGAGCAGAAAAAGAACA | |
| | CGACTATATAATATTTTAAAACATTTTAGTTTTAAGAATTAAGGATCTTGTGAATT | |
| | CACTTCCCTTCTTGAAATGTCTGACATAAAATTCTGTCAGGGATATCAGAATGGCA | |
| | CAATGAGGTTTTGCTGGACAGACTTAGCAGCTTCCTTAATTCTAGGACCACATACA | |
| | AATAAGTGGCTTTGGGGCCTCAGCCTTTTGTCTATGGTAATCCTGAAACATAAGTA | |
| | GAGAGAAGAAAAAAAAAGGGAAATACTAAATGGGTAAATATCTATACAAAATCAAG | |
| | ATAATAAAGGCCCTTTCAGGCTTGAAACTATAGGCAACAACCTTAGAACAAAAGAA | |
| | AACAAATGAACATCAAAAAACTAAAACTTTAGTGCTCTTAAATCTCAATGAAAATA | |
| | AAAAGTAAATGGTAAACTGAAAGAAATGGAAAAAAAATATGAGACTGTGAAGGGTT | |
| | AATGTCCTTTCCACGTAAAAAGCCCTTATATTTGAAGAAGAAAATAATATATTGCT | |
| | CAAAGGGAAAAAGAGAAATAAGTGAACAAAAGATATAAATAGGAAATTTACAAATG | |
| | GAGACATAAAAGTGACCAATAAACATATGAAAAATATTCAATTTCATTAATAAGCA | |
| | AAGACATGGAAATTATGACCATCTATTTTATTTTCCGTATATCGAATTTTTATTTT | |
| | AAGATCAGGCAGTATGATTAGGTTAGGGAGAAAATGTGCATTTCAAACAGTGTTGA | |
| | GAAAAGTATAAAGTGGAATAATCTTCCTAGAAAATAATCTGGCACTGTATATCAAA | |
| | GCTCTAAAAATGTAAATTCCATGTGATGTTAAAAATTCTCTTCTAGGAATTCCAAG | |
| | GAAATAATTATGATTTTTGAGGAAAAAAATCATTTCTGCAAGGATTTTCATGCTTC | |
| | TTATTTTTAGCAGGAAAATAATTTGAAAAAAATACCCAAACATCTTATAATTGGAG | |
| | ATAGTTTGCAAAAAATATGATGCATAAAAATGACATCAAATTTAAAAATTATACTA | |
| | TAGGAAGAGTGCAATAATGTAGAATGATATTTTAATTTAAAATTGTGAGAAATCAG | |
| | TTGCAAACAATAGTCAGGTCCTAAAATACATTTAGTTTCAAAGATCACAATTTACA | |
| | AATGTTTATTTATAAGTGATGAGATTACTCCTGACTTTATACTCTTCTGATTTTTG | |
| | GCTCAACCTTATAAACTCTTCTTTGAATTATTTTGTAAGGAGGAAATGATAACAAT | |
| | TAGATTTAAAAGAGTAGAGATAAAGGGACAAGGGACCATGAAGAGAATGGAAATAA | |
| | AGAAAGGAAGCAGAGAAAGCAAAAAGCAGAGCTCACTTGGTAAGGCACCCTGGAGC | |
| | CAGCAAATTATTTTTACCACATGTATTAGTTCCTTCTCACACTGATTTAAAGATAC | |
| | TCTTCGAGACTGGGTAATTTATTAAGGAAAGAGGTTTAACTGACTCACAGTTCTAC | |
| | ATGGCTGGGGAGGCCTCAGGAAACTTACAATCATGGTGGAAGGCAAAGGGGAAGGA | |
| | ACGACCTTCTTCCCATGGTGGCAGGAGAGAGAAGTGCAAGCAGGGAAATGCCAGAT | |
| | ACTTATAAAACCATCTGATCTCATGAGAACTCACCCACTATCATGAGAACAGCATG | |
| | GGGGAAACCACCCCCATGATCCAATCACCTCCCACTAGGTCTTTCCCTCAACACCT | |
| | GGGATTATAATTCAAGATGAGATTTGGATGGAGAAACAAAGCCTAACCATACCAAC | |
| | ACATATTGCTTTATTTGATATTTGACAGGTGTTTCTGTCCCTGTTTTGTGGGCAAG | |
| | TAGCTAAAGTTCCAGAGAAAACAGTTTTTCATAGCTCGTCAATGACAGACTTATTC | |
| | TCCAAGTCACATTTGATGGTTCCAAGACCAGTCTTTATTCTTGGTGGAGTTGGGCT | |
| | GAGAAGAAAGAGGAGAAGAAAGAAGAAAAGAAAGCTTCCTTAGAAACTATGATTTG | |
| | ACAGTGTAAGTAGGACTATTTCCTCCAGAAGTAACCATAAGAAGATATTAAATGCC | |
| | TATTACAGTCTTATCCCCTTAGATTTATTTAACACTTATAAAGCAATTATCATGTT | |
| | CCAGACACTATTTTAAGTATATTACGAGTATTATAGCATTGAAGGCTCAGAGCGGC | |
| | CCAAATAAATCGATCATATTATTAAACCTATTTTACACAGGAGAAACTGAGGTACA | |
| | CGCCAGGTGAATAACCTTGCCTAGGGATGCACAATTCATAAGTGATAGAGATGGGA | |
| | TTCAGACAGAGGTATTCTGTCTCCAGAATCTGGGCTCCTCACCACTTTGCAAGAGC | |
| | TTTAATTTCAGAAACTCCTATGAAGTGTCATGAGGAGAAGCCCATTATGATCCCCT | |
| | AGAAGTAATTATAGTTTTAGGAGCATGCAAAGCAGACCCCTCAGGAAGATAAGTTA | |
| | CACAATAGACATTTGGATAAGGTGGATCCAGCAGAACAAAGAGAGGGTGGTGACAT | |
| | CGAGATTGCAGAGGAATTGGAGAAGGCAATGGAAGTGTACACATGTTGCCCTCAAA | |
| | AACATAGGGTCCTCCATTGGGTTCCTATCAGGGCAGCAACATCAGAGTTTCTATTC | |
| | TGTATTTATACTAGAAACCTCTCTCCAGGGTTTCTAAGTTTTCACCTATGTTTTAA | |
| | AGACTATCTATAGGTTATTAGTCTATTTAATATTTAGGTGTATCCAGAAAGCTGAT | |
| | GGTCATCAGCTCATAGCAGGTGTTCTTTGGCTGGTGTGTTTATGTTGTGGGACAGT | |
| | GGGTTACTTGCAAGGAAAGGATGAATGGCTGGAGTAGATGGTGCTTGTGCTCTGCA | |
| | TGTATTCCCTTCTTACTTCCCATTTCCATCAGACCTACCACTTTTTGCCTGACATT | |
| | ATCTGTTGCAACATGAGCCCATGGATAGGTGTGTTTGAAGTAGGGGAATGGGAGAG | |
| | AGGGTTCCCTAGCTAATGATGTACAGCAGTAGGTGGATAAATACCTCAGCTCTCTT | |
| | TGCTCAGGTAACTGAAGCATTTTCTAATATGGTCACCCAGTGTTCCTTGGAAGGAT | |
| | TGAGTCCCAGTTGCCCCCTGAGGTTGCCTGCCCATGAACACACCCTCTTTTATTGG | |
| | CTTCCTTCCCATTCTTTTCTCACTTCCCCATTCCTTCAATTCATTGAGATTGTTTC | |
| | CAAATAAGATGACTTGCTCTCACATCTCTGTGTCATTTTTGGCTTCTTGAAGTATG | |
| | CAAACCAGGATAATAGCTAACTGAAGGCTATAGATAGCCACAGGCAAATTTAAGTA | |
| | ACAGTGTAAGAATATTCATACTTGGCAGAGATTTATTTATAAAAACTCAGAAAATT | |
| | CACATGGAATTATGAAGTTATTATTGTATTTATTCCATCATTCCCAGAAAGAATAT | |
| | GGAAATCCTCTCAAGCAAGCCAGTCCTTGGGAATATTGGGAAATCTATGCAATTTG | |
| | TTGTGGAGTATTTTTTTTTTTGTTACCCTCCTAAATATCTGGCCGCTAAGCATTCC | |
| | TGTCTCCAGGGACTTAGACCCTAGCAAGGAAGAGAAGTTGGGGCCAGGTTCAGAAA | |
| | ACGGGTTAGTTATCAATCTCCCTGGAGAAGTGTCCCCCTCAGCAGGGTCAGTGAGA | |
| | GTAAGTGAAACCCATTGGTGCCCACAGGCAATGGTCTGGCCTGAGTAATTAGAATG | |
| | GGCCTCCAGAAAGTTCTGGGAATTGCTATGGTGCCATAGTCTCATTTTCCCCGTTG | |
| | ACTCTCCAGATTTATTCAGAGTCCAACTTCAAGGGCCTTTCTGCCCTTCCTCTCAC | |
| | AACTGTGGAATAATAATAATCCACCTTATTAACTGGGACCGAGAACTGAGCTCGAC | |
| | TCTTATTTTTTTGAGACAGAGTCTTGCTCTGTCACCAGACTGGAGTGCAGTGGCAC | |
| | TATCTCAGCTCACTGCAACCTCTGCCTCCCAGGTTCAAGCGATTCCCCTGCCTCAG | |
| | CCTCCTGGGTAGCTAGGACTATAGGCACGCACCGCGACGGCTGGCTAATTTTTTGT | |
| | ATTTTAGTATAGACAGGGTTTCACCATGTTGGCCAGGATGGTCTTGATCTCCTGAC | |
| | CTCATGATCTGCCTGCCTTGGCCTCCCAAAGTGCTGGGATTACATGCGTGAGCCAC | |
| | CGCGCCCTGTCTGAACTCTACTTTTTTACACTGCTGCATGTTTGTAGAGTGACCAA | |
| | TGAAGCTATACTTTTTTCATTTTCAAAATGATGATGAATACAAGGTTATCAAATAA | |
| | AACACAGAGGGCCCATTATGTTTGAATTTCAGATAAACAACAAATCATAGGTGTCC | |
| | TGTATGTTTGCTCAATCTGGCAACCCTGGATGAATAAGAGCTCTCACCTGAGGATT | |
| | TCTTGTGAGGATTCATGAAATAAATGCTAGAAATGCTTACACACTATCTTTATTTG | |
| | CCCCTCAGAGCCCAAAGTCTCTGAAATCTTTATCTTTCACACACAAAAACTCACTT | |
| | TCAGAAAAGTATATTCCATTTACATCTAGTGGAAATAAAAATTGTTCTTTTTCTTT | |
| | GTGAAAAATATTTTTATTTTAAGCTTTATGCAGAAACCTCAGGGAAAAAAAGGTAC | |
| | TTTTAGGAGCCAGGCTTGTAATGTAAATGTCCAAAAAAGATGAAATTGAAACAAAC | |
| | AAACAAACAAACAAACAAACAAACAAACAAAAAACAGTGCAAGCTCCTGTGTGGAG | |
| | ACTGCAGTGAGTCTGAGATTGCATGTTCCATCAGAAGGGGGCAGCCACATCTTAGC | |
| | TCTTGATGACCCAAGGGAGCAGGGATGTGGGGTTGCCAAATCTTCCAAAATTTTAA | |
| | GAAGCCAGAAATCTTGATTTCTATGTACAATCTCCTGGTTTTTAAATGTGGGCAAA | |
| | TAAATCAAAATTCCCTAAAACACTGTTTGGGGCAACAATGTGTGGGCCAAAGTAAA | |
| | TACTTTTGTGGGCTACAAGTGTCCCCTAGGCTGTACATCTGGGACATCTGATTTAT | |
| | GTGGAAATTTACCGAGAACTAGTTTTATTTCTGTGGCAGGTCATTTTCACTTTCTA | |
| | GGATTATGTTTCTTCATTGATAAAGTGAGCTACTTGAGCAAGACCAGTGGATTGAA | |
| | TGCCACGTCCCAAGGAGGCTGGGGTTGTTTCCAGGGATCTTACAGAACTTAGGTGT | |
| | GATACTGAGCATGAGCTACTTGTGTTGCATTTTGGTGTTCAAAAGAAAAGTTCTTT | |
| | AAATAGTTCTGCTGGAAAGACAAAAAAAAAAAAAAGAAAAAACTTTCACAACAAAA | |
| | ATCTCCAAAAACAAAAACCCAGAAAACTGGCATAGAAGTGGATGATCTTTGCAATT | |
| | TTTTTCAGTATATAAATAAATGATTTTGATCCCATTTAAAATTTTATCAAATGCAA | |
| | AAAGAAACAATTCAAAGTATAGAGCTACCTTTTCTTACTCTACTGAAATCTACACT | |
| | TTATGTCAGCCCTGGAGGGTTTAGACGCACTTTATGTCAGCCCACTTCTTTCGACT | |
| | GCACTATGTCAGCCTTGGAGGGTTTAGATGAGGCAGTGAGCATTTGAATGCTTTTA | |
| | ATTTCCATTTTTCAAAGTACATTCTTGGTCTATAGGAGAGGAACAAGATATGTAAC | |
| | TATCTCTGACTATTGCTAAAAACACAAACGTCTTTAATAAATGTTGCATAAACTCA | |
| | GAAAGTGATACTTCAAAGTCTTGTGAAAAATGATGATCACCAGCATTTATACAGCA | |
| | ATTAGTATGTGCCACGCAATTTGACTTTATTATTTATTCATCTATCTTTACCACCA | |
| | TCTTAAAATATGTGAGTGCAAAACCCTGAGAAACTTTCTCCAACTCCTGTGGGTGT | |
| | GGAAATCGAGGCTTAGAGAGGTTAATGCTTTGCTCAGATTATTAATCACTTAGGCA | |
| | GTGCTACCTATAATATCCTGCTCTGTTACTGGTATTTCCAAACGTCATTAACTGTA | |
| | GCAAGAATCCTAAGGCAAGCACTATGCTATCATCTTAAAATATTTATTGCAAACAT | |
| | CCTATGTTTTATTGTTTTATCTTTTTAACTTTGAGAAGATAAAATAAGCCACAGAA | |
| | GTGAAATTAATTGGGAAATCATTCGCTTTTTGCAAAATTTGGGAGCATAAACAATG | |
| | GGTCATGAATTACAATCAAACAAAAGATAAAATTCTAAGAAGTCTTTTAAAGTGGA | |
| | AAAAAATAACTGAAAAATACTGAATGGAGGGCAGTTTTTCATGCACTGTGTTACGA | |
| | ATAAAAAATTTGATTCAATGGATTACTTAATCAACATTTTAATAGTTGTAAATCTT | |
| | ATAATATTTAAGCTGTTTTATAAGTGCCTCTACTTATAATGGCACATCCGTTTGAA | |
| | ACTCTAGCAGATCATTTTTATTTATTTTTTTGAATTTTTTTCTTTATATTCTTTAA | |
| | AGAAGGATACAAAATTATTTCTATGAATATTTAACATATGGAAGGAAATAGCAATA | |
| | ATAAACATAAATGCTAACACATATAAAATAGGTGGTATCATTAGGCTAAATTTTAG | |
| | TCTTCCAGGATAAGTAGAACATCTCTGACTTCTCAAATATCCAATTAATAAAATGC | |
| | TTACTATACCATTTGGTGCTTTAAGAACATTGCCATGGAAACCTCTCAGGTTTTAT | |
| | GCACAGTAGCTATAATAAAATTTTCCTTCATCTTTCATGGAGCTACTTGAGATTTT | |
| | TTTTCTCCCTTTAAACATGAGAAATCAAAAAGAAAGAGAAAAGAAGGATTAAATAT | |
| | TCATTTATCCTTTTGCTTCTGACTTGTTATGTGGGCAAGTGCCACATGAGGGAGTG | |
| | CTGGGACCTCATATCAAGAAAAATTAAAACCTACCTAATGCGTTCCAGGAATGTTC | |
| | AGCATATTAGCAAATTCTTATTAAACTGTCAAAAAAAAAAAAAGTTTTAAAAGAAA | |
| | TTCCAGCCCCTGGATGCAATTAGAGGCTACCACACTGGATTTGATGGGCCATAAAA | |
| | CCATTAAATCTAAACACTTTCTTTTTGAGCCTAAAAGGCCAGAACATTCCAAAGTG | |
| | AAGTTTTGGGACTCAGCTATGACTTGACCACCTATTAAGATGCAGGTGGAACAGAT | |
| | TGCAGAGTAACACAAAGAGCCACACAGACCCCAGATGACTGCATTAGGGTGTAGGT | |
| | GAGAGTTTTAGCTGTTGAATTTTCTGGATTTTCCAAGATTAAGTGATCAACCTTAA | |
| | CAATGAGTGAAAGACCATTCAACAGGAAGAATTGTCATTTCCTTTGCTCTAAACCC | |
| | AAACGATGTATTTTTTGAAAGCTTTATTGATTTATATATTTATGTGTTGTGCTAGG | |
| | CGACCGACTAGATATATGTTTCAGCATACCTACTAGGAAAATATCCCCATTATTCT | |
| | CAATTTTACCTAATCCAGGCAAAGCACTGGACTTGCTTTAAGGAACATTTTTACTC | |
| | TTTCTGAAGTGGAGTGCCTGTCATGTATCAGGTGCAATGCTTGGACTTTACGTTCT | |
| | TGTGATTAATCCTTACAATAGGCCTGTGAAGTAATTCTCATTCTGTTTGACAGTAG | |
| | AGAAGAAGGAAGCCCTTGACCAAGGTCTAGTGCCAGTAATGGTGGTGATGGGGTTT | |
| | GAACCTAAGTCTGTTTCACTCTAAAGTGTAACCAAATTTTATGTTTTAGACTTGCT | |
| | TTTCTAACAATAAAAAGTCAGTGACATGCTCTTTCTGTGTGTAAGCACTCACACAC | |
| | ACACACACAAACACACATCCGTATTACATATGCTTATATATGTATTAAAAGATTAT | |
| | GGACATTTGATATATATACATATACTAAAATGTATAATTCATTGCTAAAGTATTTT | |
| | CATATAAATAGTGGCTTCAGTGTTAAAATCACTTTGCAATGAAACAAGATTGTTGA | |
| | TTAAAACACCTATTAAAAAATTAGAATCTAGCCATATTAAAGACAGTCATCGAATG | |
| | GAGTGATTTCTACGATTTTGCACCAAAATTTAAGCTATTGGGtGGCTTTCTTGAGA | |
| | GCATGAGATTGCTTCTTCTCAGAATTATTAATGTGCCTGATGACATTAAAATGTGA | |
| | CAGTGAAAAAAGTCAGAGGCTCACATGTGTATCCCAACACTGAAGTTGTTAAACAC | |
| | TGGGAGGTTGGTTGAAGTTGTTGTGTGCAAACTCAATACTCCTTAAAACCATTATT | |
| | TAAAGGCCTATCACTGTGTTATGGTCTCCATATGATCTGCCATTTATGCCAGGACT | |
| | TGACAATTCAGTAAAATGACAGAATAATAACACAGGAATCACTGCAGTAGAGCTAA | |
| | TGTTTTAGTCTGTTGCAGAGTTCTGCCCTAGAAATACAGTGAAAACAAGGAAGGGA | |
| | GAGCTAAGATGTCCCTGAGACTAATTGTTCCTTGAAAATATTTTCATAAGTAAAAA | |
| | AGAGGTCTAGAGGTGTAGTGGCAGTGTGATCACTCAAGATTATATAGCTCCGGATT | |
| | CGTTCAATGGGCCATGATGAAAGCACGGCAACGATTAAATCTGGTTTCTTGGTCTT | |
| | TCTTGGCAGTGTTTAAATTGGTTCAGTTCCATAAATTGTAAATTAAGATCTGTTTG | |
| | ACAACTTTTAAGTATTTCAAGCATAATTGTAGTTGAAGGTTTGTTCTTTTAGATCA | |
| | CTGACTTCAGAACTTTATTTTTCTGGTTAATCTCAATTGTAATTTTAGACATTCAT | |
| | AAAACAATGTTGACTGCGTCTATGTGATGGTAGATCCTCTGTGAAGACCTTTATGA | |
| | TGGTAGTTCCCCTGTGAAGATAGGATGACACACTCAATGGACATTATGGTGCACAG | |
| | TTATACAAACACTTCACTATGACAGGCCCTGAGTTTAGAACCACACAACTGCTTGG | |
| | TACTTGGTCATCGCATATTTTCCCCATTACGTAATGACTTCCTGTGCAGATGACAA | |
| | AATGCGTTTTCTCAACAAAATTATTTTCAGTGCAGCTGTTTTGATGACTAAGTTTT | |
| | GTAGGAGCTTTTTAATCAAATGCACCTAAGAAAACCCCAACACTTTAGGCCCTTTG | |
| | AACATATTACACTTTTTTGCTTCCTCTTTCCTCTTTTTCCTTAAAACCATAATTTG | |
| | GAAATTTGATTCTGCCTTCCCATAAAAGAGAATTATTTTCAAAGAAATTATTTGGG | |
| | TCTAAATTAACATGTTACTTAATTGTTCTGCTTGAATCTAGGTATATGATTAGTCC | |
| | CATATGAATTGATGTTCCAAATAATTTACTCTCATTGATAACTAATATTTTCTATT | |
| | TCCCTCTATTGTTTTGTGGTGGTGGTGGTGGCTGTGGATGAACATCATTCTCAAAT | |
| | ATATTATAATTCCCTTCCTCATCAAGCCCAGCATGATAAACTTCAGTTTTGCCTGA | |
| | TGGTTCATCATCTTATTTCTGTGTGTAAGATTGTTGGATTTGACATTAAACATTTG | |
| | GAAACTATTTTATAATTGATAACTTGTGCTTTCTCAGCTTTGAGTAAGCGCTCTCT | |
| | TCTTCATCTTATACCATTTTATTTTTATTTATTATTCACTTCTGCTTCTGATCTGA | |
| | GATCTAGGAAGCTGGACAAATCCCAGATAAGCAAGCTAAACAAACAAACAACAACA | |
| | ACAACAACAACAACAACAACAACACAACCCAAACTAAACCAAACCAAAATCATGGG | |
| | ATAATGGTTAAGTGTACTGAGGGGCCATTATGCGAACACAGTTTAATTCCTTGGCT | |
| | TTAAAACTAATAAGAGAAGAATACATAAACAAATGTGGCAAATGTACCTGTGACCC | |
| | TCTCCAGAGGGTGCCAGGCTAGAAGAAAGGCAGATTTATCAGCAAGGCATGGCGGG | |
| | CCATTGGCAAACCATGGGACAACCACTACCAACTTCACTGCCATTGCTCCATATTT | |
| | CCCTCCCGTTTTCAATGAGCCCCAACTTTGCTCAGGACATCACACATATTCTACTA | |
| | ATTTGGATGAGTCCTTTTGAAAGAAAATATCTACCTCATGGTTCTCAAAGTATGGT | |
| | CCTTGGAACATCAGCGTCAGCAGGACTCTGGAGCTTGTTAGAAATGCAGATCTTAG | |
| | GTCGCACTACAGACCTACTGAGTCAGAATCTGAATTTTGTTAACATACCCATGTGA | |
| | TTCCTCAAAGATTGAGAAGCCCTGATCAGAGCCTGGGATGAAAGTTCCTGTTGGTT | |
| | CCAAGCCAAAGGCATAGTTCAGGTCTTCACACATGACACTATTAGATGTAGATGGA | |
| | TATTGTTCCCTTCTGAAGACCCTCAAGGTCTTCTGAGAGCCTATTAAGTTCAGAAT | |
| | GACTGCCTGAAATGAGTGAGAAGTCACAAGGAGACTCTAGATAATTAAGAGATGTG | |
| | TTCACAGTAGTCTTTGATAAAAACCTGGGACAGGCAGGCTTAGTATGCAGGCCCCT | |
| | AAAATTTATGTACACAATGGATTTCCTATTTTTGCTTCTTCACATCCAGATTACCT | |
| | GGATCAGAAATAAATGTTTTCATTAAGACTTGATGTGACAAACAAACAAAACAAAA | |
| | CTCTGCCAAGCTCTAGAAGAACAATTGCATTTCCCAGCCAGAGGGAGAACACTGCC | |
| | AGTTTTTGCTGTTTTCCAAAGCTGTTTACCTGTCCTAGCTCATTTAAATCACTGTA | |
| | CTTTGGAGTTCCGGATTAGCGTCCCCAGAGGTAGCTGCATTCATACTTGATGAGTT | |
| | CTTTTAAATCTCAGCCATTGATTGTAGGTTCCATAGTATAGGAAATTTAGCCAACC | |
| | CTCTATTGAATGGCAGTTTAGAAAGGTCGAGCTACACTTACCTTATGTCAGGTTAT | |
| | TGCAGACCCTTGTGGCATTTTTCCACCCTAGGACATGTGATTTAACTCTAATAGAA | |
| | ATCTTTATTATGGGTGGGTCTGAGATTAACTTTTATTCTATAAAACAGAAATCATG | |
| | CCACTGGCCGTAGCCCATTTTTTGAGATGGAGTGGGGGGAATGGATGATAGTAAAC | |
| | AAGGATATTAATCTCATTTATTTTTATATCATTATATTTATAGTTACATTGCAAAT | |
| | GGAAGAGTAGAGAAACCAAAAACTTACACTGGGAACTTTACAATTTTTCTTCCAAG | |
| | TATTACTGATTGATGTTTGGACTATGCAAGTGCTGCCAGCCCCTTAGACTCACTCT | |
| | GCAGCTCCCCCCATGGAAATTTGTGAACAGGTTAGGGTGGGGATAGGGAAAAGCAT | |
| | GTTCTTGTTTCACTTCTTGGATTATTTGTTCCAGGCTCTCCAAAGTAATGTGTACC | |
| | TTGGGAATGCAGAAATTATCTCCTTAGATATTCTCTCCCTATATATGTCCTCACAG | |
| | GGAATTCTTGGAATTGGAGAAGATTCCACTCTCCTTTAGGAGCTTTCTCCATAAAG | |
| | GTATTGAGCATTGGACACTATATTTGCAAGGGAAAAGAGGAATGGGTCTCTTGAGC | |
| | ATCAAAATCATTGTAGAAGAATCTCCAAACTGTTTTTCAAAATGTCTGTACTAACT | |
| | TACATTCCTGACATCAATGGGTTCCCTTTTCTCCACAAGGGTTCCCTTTTCTTTGC | |
| | ATCTTCACCAACACTTGTTATCATTGGTGTTTTTGATAATAACCATTCTAACAGTT | |
| | GGAGGTGATACTTCATTATGATTTTAATTTAAATTTCCCTGATAATTAGTGATACT | |
| | GAGCTTCTTTCATATATCTATTGGCCATTTATATCTCTTCTTTTGAGAAATGTCTG | |
| | TTCAGATCCTTTGCCAATTTTTTTTCTTTTTTCAACTTTTATTTTAGAATCAGGGA | |
| | GCCATGTGCAGGTTTGTTACAAAGGTATATTGCATGATGCTGAGGTTTGGAGTGCA | |
| | AATGAATCCATCACCTAGGTAGTGACCACAATTCCAAACAGGTAGTTTTTTTCAGC | |
| | CCTTTTCCCCCTCCCAACCCCACTGTTGTATTCCCCAGCATCTATTGTTACCATTT | |
| | TTTTGACCATGTGTATCCAATATTCAGCTTCCATTTATAAGTGACAACATGTGGTA | |
| | TTTGGTTTTTGGTTACTACATTAATTCACTTAGGTTATTGATTTCCAGCTGCATCC | |
| | ATGTTGGTGCAAAGGACATTATTTTGTTATTTTTTATGGCTACATAGTATTCCATG | |
| | GTGGATATGTACCACATTTTAAAAATTCAATCCACCATTGGTGGGCACCTGGATTG | |
| | ATTCCATGTCTTTGCTATTGTGAATAGTGCTGTGATGAACATGCAGGTGCACGTGT | |
| | CTCTTTGGTAGAATGACTTATTGTCCTTTGGGAATATACCCAGTTAGTGGGATTGC | |
| | TGGATCGAATGGTAGAAAAACTCTCAGGTCTTTGAGAAATCTCCAAACTGCTCTCT | |
| | ATAGTGGCTTATTTAATTTACATTCCCTACAGCAGTGTATCAGCCTTCTCTTTTCT | |
| | CCACAGACTCACCAACATAGTATTTTTTGACTTTTTAACAAAAGTAATTCTGACTG | |
| | GTATGAGATGGTATATCATTGTGGTTTTGATTTGCATTTCTTTGATGATTAGAGAT | |
| | GATGAGCATCATTTTCATATATTTATCAGCCTCTTTTATGCCTTTGTTTGAGAAGT | |
| | ATCTGCAAATGTCCTTTGCCCACTTTTTAATGGGGTTATCTGTTTTGTCATGTTGA | |
| | TTTGTTTAAGTTTCTTAAAGATTCTGGATATTAGACCTTTGTTGGATGCATAGTAT | |
| | GCAAATATTTTCTCCAATTTTGTAGGTTGCCTGTTTACTCCTTTGATTGTTTCTCT | |
| | TGCTGTCCTTTGCCTATTTTTTAATTGGGTTATTTGTTTTCTGGCTATTGAGTTGT | |
| | TTGAGTTCCTTATTTTTTTTTTTGGATATTAGCACTCATTAGATATACACTTTACA | |
| | AATATTTTCTCCCAATACCTGTGTTGTCTCTTGATTCTGTTAATTGTTTTCTTTGC | |
| | TGTGCAGAAACATTTTAGTTTCACACAATTCCTTAAAAAACTAAAAATAGAATTGC | |
| | CATATGATCCAGAAATTCTACTTCTGGATATTTATTGAGAGGAATTGAAATCAGCA | |
| | TGTTGAAGAGATATCTGCACTTCTATGTTCGTTATAGCATTATTCATAATAGTCAT | |
| | GATATGCCATCAACCTAAGTATCCATTGACAGATGAATGGATAAAGAATGAGGTGT | |
| | ATGTACACAAAGGAATACTATTCAGCCTTTAAAAAGTGGGAAATTCTGTAACAACA | |
| | TGGATAGACAGATACTATATGATCTTACTTATATGTGGAATCTAAAAAGGTAGGTC | |
| | TCACAGAAACAGATCATAAAAAGGTGGCTACCAGAGGCTGGGAGAGGAAGGAAAAG | |
| | AATGAGGAAAGTGACATATTGATCAAAGTTGTACAAAGTTTCAGTGCGACTGGAGT | |
| | AATAGGTTTTAGTGATCTATTGTACTGCATGGTGTCCACAGTTAATAGTAATGTAT | |
| | TGTATATCTTAAAATTACTAAACGATTAGGTATTTAATGTTCTCCCTACAAAAAAA | |
| | TGGTAAGTTGGTGTATTAGTCCACTTTCACACTGCTATAAGGAACTGCCCGAGACT | |
| | AAGTAATTTATAAAGAAAAGAGGTTTACTGGCTCACAGTTCTGTATGGCTGGGGAG | |
| | GCCTCAGGAAGCTTACAATCATGGTGAAAGGGAAAGCAGGTATGTCTTACATGGTG | |
| | GCAGGTAAGAGATCCTGTGTGTGAAGTGAAGGGGGAAGAGTCCCTTATAAAACCAT | |
| | CAGATCTCGTGTGAGCTCACTCACTAGCATGAAAACAGCATGGAGGAAATCACCCC | |
| | TATGATCCAATCACCTCTTTCCCTCAACACATGGGGATTACAGTTCCCTGCCTTGA | |
| | TGCGTGGGATTAAAATTTGAGATAAGATTTGGGTGGGGACACAAAGCCAAACCTTA | |
| | TCAGTTGGTGAGGTGATGAATATGGTCATTAGCTTGTCTAAATTTGTCTGCAATGT | |
| | ATACATAGATCAAAACATCACTTTGTACCCCATAAACATGTGCAATTACTATTTTC | |
| | CAATTAAAAATAAATATAAATAAATTAAAAATAATTGCAAAGGAAAGCTGGCTGTG | |
| | GAGAAGATTAACAAATAATGACATTAAGAAATTCAGGTCCTTGGCAAAATTAGAAA | |
| | TACATACAAAGCTATCCAGAACTTATTTTTCCAAATGCATTAGGCGTCCTCTCACC | |
| | TTACCCTTTACAATTGCATGGCTTCAGAGATTACACAGAAAACGTTCAGAAACATT | |
| | GCCCCAGTAGATGATCTTGCAATGCTATGAAGTAGGCAGAACAGCTGTGGCTATAG | |
| | CAATTGTGCAGATAGAACGTACTTCATGGATGGCAAGACTGGGACTCTAGGACAGG | |
| | CTTTCAATCCATTCTACCCTGTTGTTGTTCTGAAATGAAAGTTTTATCTCCCAGTT | |
| | TATATAGGTAGCCTTATCTTTGATGCTTCAATACCTGAGACCTGGCCAGTGTCCCT | |
| | TTTAGTGATTGTATGTGTGTGTGTGTGTGTCATATGCAATTTCCTTATAGCAATGG | |
| | CACAGTGTATCACTGTTTAATTAAAGAAGAGAAAGAAATGCCAAACATACGAATAA | |
| | AGTCTGAATATATCTGTAACATTAAAAGTGTAGGTGTCTATCTTTGAAGATATGTC | |
| | TTAAGGACAATGAAAGAGTCAGTGAGTAAGAGAAGAGAGTCCTGGGATTTCATACA | |
| | AGATCAGTGTTACTTGATGGTGTAGGCTCCTAGGTATTTCATCTTTAGGATATACC | |
| | GTCTATTACAAAAGCCAAGATTTTTAGATTTGGATCAACATTAGGGAACTTCATTC | |
| | TAGGCAAGAGCCAGGTTTTGCCTTTATGTTAATATGACCTCAGCTGTGAGCTCCAT | |
| | TTTGCCAGGCATCTTAAAACTGCAACACATATCATTGGAATCTTCCGTTACAGTCT | |
| | AATACATAGCCACACATTGGGAGCAAGAATGAAATCCAACCCCTGTCCTTTGCAAA | |
| | ATGCAATGAGACAGTGTCTGCTTTGGGAGCAGGGAGTCAGAATTTCATTGTGGACA | |
| | ATGGATAAGGTGAGTAAAAGGGCTTAAAACATTTGTGCTTTCAAGCCATAGGCTAG | |
| | GATAACGATAGTCAGAACTTTTTGATGAAGTCTGACCATGCTACGCCATTTATAAA | |
| | ATTTTGAAGCTTGTAAGTATTACCCCAAAATGAGCAGTGTGAACTCAAAGGGTTTA | |
| | TCATTGTCTCTCAGGCAAAGGTAATATTTGAATTATTTAGCAAAGGACTTTGAGCA | |
| | ATTGGAAGAGATACTCAGCTGCTGGTCTCTAGCGCTCTAACAGGGTGGATGCCCCC | |
| | CGCTCTGCCGGCACTGATGTTTAAGTTGCTGGATTATGAGGAAGTCTGGGGATTCC | |
| | TTGGGGAGAAAAGGAAGTGATGACATATTGAAGCACAACGACATATTGAAGAGACT | |
| | CGGGGGCTGGGGTGATAAACTTCAGAGCCGTGGCTATTTACCAATTGGAGTGTAAG | |
| | TATTTTAATATTTTAACAAACATAATTGCCATTCTGGTATGTACCAACTTCATCTC | |
| | AGATCTGTCCTTAAGAAATAGGCAAATTCTTTATTGCCTCTCTGAATGGTTCATAT | |
| | AAATTCCCAGGCTCCCTTAGCTCATTCTAACATAAAACTGTATTAAAAATAATGAA | |
| | TGTAATTCATCAATAATTTTCCTTTGTCATAGCAAATAGTCACAAGTGGATTGAGA | |
| | TCAGAGTGATCACTCATATTTGTTCTGGGGAGAAGGGAGCCTGCTGTTTTGCTCCT | |
| | GTTTTCTCCTAGGACTAGTATTTTAGCTTCAAATGATAATACCTTAGCACAGACTC | |
| | TGATATTCCTCCTACATGCAGGAGCATTCTCTTGGAATAATTTTGGGGATGCCAAT | |
| | TCAAAATTTCAGCCATGTATGATTTACTTATTGGAAAATAATCACTGAGCAGCAAT | |
| | AACTCCAGCAGTTACTTGTATCAAGGTAGAATCAAGAAATAGATGGTATGGACCAA | |
| | ACTTGCTTCTCTCTAAATATGCATACCCAAGTGATTTGGGTAAAATGTTTGTGAAG | |
| | GGCTTACATTTCCTGCAAGTCAGATGGTTTAAGAGAAGTAGAAATTATGTGTGTTT | |
| | TGCAGCATTTTGGTAATCTGTGTGGAGTGTCTGTAGATATTTCTCATGAGTTCAAG | |
| | GGAATCCTTTTGTGGATTTTGATGTTCCTATTGGCAGAGCTGCTGCTTGACTACAT | |
| | GATGTCTTTGTATTAACTACAAAAACATGCCCTATCATCTGAGTGATTTTCTCTGC | |
| | CAGACCCCTTTGTGCATCCACACTCTGCACCTCCAGTGTACGGAGGACCTTCCCAC | |
| | TGGATTCTAAGATTCCATGCCTTCCCAATGCATGGCAGTGTCTCTCATGCACATGG | |
| | CAAACCTACTCTCTTGGATGTCACTGCCCTGAAATATTGAGGGAGTACATTTATCT | |
| | AGGCATGGTACCAGGGAGTCATTTAGACATGTAGGGAGTCTAGAAAGATCATTGCC | |
| | CTGGGAGAGTGCTCAGCCATGCTGAGTTCTCCTACTTTGTTGCTCATTTCTGTGTG | |
| | ACCTTAGGTAACATCCTCTTCAGGACTTTTTTTTTTTTTTTTTTTTTGACAGGGAG | |
| | TCTCATTCTGTCATCCAGGCTGGAGTACAGTGGTGTGATCTCAGCTCACTGCAATC | |
| | TCCGCCTCCTGGGTTCAAGCAATCCTAGTGCTTCAGTCGCCTGAGTAGCTGGGATT | |
| | ACAGGCATGCGCCACTACGCCCAGCTAATATTTGTATTTTCAGTAGAGATAGGGTT | |
| | TTATCATGTTGATCAGGCTGGTCTTGAACTCCTGACCTCAAGGGATCTGCCTACCT | |
| | TGGCCTCCCAAAGTGCTGGGATTACAGATGAGAGCCACCAACCCTGGCCAGGACAT | |
| | AATTTATTTCAGGTGAATTGATTGTTGGAGGATTTTGATCCAAGCAATCAATGTCC | |
| | CTTGGTGTTCCTTTCAAACAGCAGTAAGTGACCTGAATTTATTTTCCACATTTCCA | |
| | AATCTTAATGAAAATCAGACAATGGTCTATATGTTCATTTGTGTTCTTACTTAATA | |
| | AAATGTGGGTTTTAGACAATATTTTGCCAGTCATGAATTCCTATAGAAGGAACTCT | |
| | TTGGGAGAACAGACTAGTGATCTATAGACATGATGACCTCCAACTCAGATCTTCTG | |
| | TAGCTAACCACTGACCGGGAGAACATGTATGAAAAACATCTTCAAAGGCATTGAAA | |
| | AATTAACATTTATCAAAAACAAAATACATTTTATTTCATTTGAACTTAGACCTTTA | |
| | CTATCTAATGGCTATGGTACTATTTAAATGTCAAAGTGTGATCTAGCATCAGCCTA | |
| | ATCTGGTTAGAAATGCAAACTCTTGGGCCACATCTCAGACTTACTGGACCAGAAGC | |
| | TCTGTGGGTGGGACCCAGAAATCTGTGTTTCATTCACATGCCCTCCAGGGGATTGT | |
| | CCTGCTAAAGTTTGAGAATCATGGAAGCTTTTTAACCTCTCATTATAGCTTTATAA | |
| | GCAGCAACTCACTGGATTCCTATCAACATCCTGTGAGTGTCATTTGGACAAGTATA | |
| | TTTATACCCATTTGATGCATGGTAGGCACACAGATGAGTCAAATGACTTGAAGGAA | |
| | TAGAGTTTTACATAATATACTTTTATATATTTATACTTCTAATATATTTATACTTT | |
| | ATAACAGATTTGACTGTTTTATATATTGCATATAAACATTATATCAGTTTCTCCTC | |
| | CACTAAGGCTGACTCCAATTTTACTCCAATTTTACTACCAATTTTTGGAAGAAAGC | |
| | CTACCTATCACTCATGTTCTCTCAAGTACCCTCTAAAACTATTAGTTAGATGACTC | |
| | TATTTAATTTTCCATTTATTTGCCCGTTTCTTGCTACCTTTCCCCCCAAAATGTAA | |
| | CTGCTACCTTGCTCAAAAGGATGTGTCTACTTGGGATATCTAGCACACACATTTTA | |
| | TGAGATTTTAAAAGACAACATAAATGGTAAACTATATATTTAATACAATTTTGAAA | |
| | GACAAAATTTTAAAATTAAAAAGGAAGAAAAAAATTAAACTAACCCCATAATTCTC | |
| | CCACCCATCATTAGCATGTAGTTTGTTTAGATTCATCTACCAATAAGTAGAATTGT | |
| | ACAAATTTGATATCATGTAATACATGTCATTTTGTAAACTTTTTTCTTTCCTTAAT | |
| | ATATCTATATATCATAAACATTTTTCTATGTCTATATTATTTTAAAATTGTAATAC | |
| | CCAGAGTTCTCCAGAGAAACAGAATTAATAGGATCTCCCCCTTGGAGATTTCTCAT | |
| | CTTTTTCTCTCTCGATAGATACAGATAGATACATACATAAGTCTATCTCTCTATCT | |
| | CTATCTTTATCTCTAAAACACCTATCCATAGATAGACATTTTTAGGAATTGGCTCA | |
| | TGTGTTTGTGGAAGCTTGCAAGTTCAAATGTGCAGAGTAGGTGGGCAAGCTACCAG | |
| | GGAAATGTTGATGTTGCAGTTCCAGTCTGAAGGCAGGCTCCTTGCAGAATTCTTCT | |
| | TTTTCTTAGCAGTCTTACTTCCCCTTCCTCTTCCCCTTCTCCTTCCCCTTTCCCTT | |
| | CTTCTTCTCCTTCCCCTTCTTCTTATTCTCCTTCACAGACTTATTTTTAAGGCCTT | |
| | GAGCTGATTAGATAAGACCCACTCACATTATGAGGGATAATCTGCTTTACCTGTAG | |
| | TCTACTAATTAAAATGTTAATCTCATCTAAAAAACACCTTCATAGCAGCATTCAGA | |
| | CATGTTTCACCAAATATCTGGGCACCATGGTTTAGCATATTGATGCAGAAAATTGA | |
| | TTATCATAATAATATTATTTTTTTTTGAGATGTAGTTTCACTCTTGTCACCCAGGC | |
| | TAGAGCGCAATGCTGCAATCTCAGCTCACTTCAACCTCTTCCTCCTAGGTTCAAGC | |
| | GATTCTCCTGCCTCAGCCTCCTGAGTAGCTGGGACTACAGGCGCCCATGACCACGC | |
| | CCGGTTAATTTTGTGTTTTTTTAGTAGAGATGGGGTTTCACCACGTTGGTCAGGCT | |
| | GGTCTCGAACTCCTGACCTCAGGTGATCCACCCGCCTCAGCCTCCCAAAGTGCTGG | |
| | GATTACAGGCGTGAGCCACTGTGCCCAGCAATAATATTAATTTTAATGGGTGTGTT | |
| | CATTTCATTTTATATATGACCTACAATTTAACCAATCCCCTAAGGCTGGATGTTCA | |
| | GGTTCTTAATATTTTTTGCCCGTATTTACAGACACCTTTGACTATTGGATTTATTT | |
| | TGTTCTTCAGGAACAATATACAAAGTGTGGAAAGAAATGTATATTTCTAATCATTG | |
| | GAAAATAAACACTGAGCAGAAATAACTCCAGTAGCCATTTGTATCAGAGGAGGTAG | |
| | AATCAGGAAATAGATGGTATGGGCCAGACTTTCTTCTCTCTTTAAGAGATTTGACT | |
| | TCATATTGCCAAATTGCCCTTCTAGATGTCTTTACTCATCCAACTACAATTCAAAG | |
| | GTTTGGGAGGGTAAGCAATGCCAGGCCCATCTTGATCATCCCCTTTCTTTCTCAGC | |
| | CTGTCAGTCCCTGGGAACGGGCATGATGTTGAGTTCCTGGGCCACCTCCTCAATTG | |
| | AAGAAGTGGCGGAAGCTGGTCCTGAGGCACTTCGTTGGCTGCAACTGTATATCTAC | |
| | AAGGACCGAGAAGTCACCAAGAAGCTAGTGCGGCAGGCAGAGAAGATGGGCTACAA | |
| | GGCCATATTTGTGACAGTGGACACACCTTACCTGGGCAACCGTCTGGATGATGTGC | |
| | GTAACAGATTCAAACTGCCGCCACAACTCAGGTAACCATGATCATGTGGGCCCCGA | |
| | GCTGAGGCGAAAGGGATCTTGACTGGGAATGTTAGGGTCTGGGTTCTACTGATAGC | |
| | AACGTTGCTAAACATCTAGTTAATCTTCAGCTAATCACATCCCTTTTGTAGACATC | |
| | ACTTTTTTTGAGATACACAATAGAAACAGAAATGGCCTCTATAAAAGTCCAATAAA | |
| | TTTTCAGACCAGAGTGCATTAAGGGCTTTGGCTTTGGGAAGTATGAATTGCTATAC | |
| | AGATGGAAGATACTGAATTTTGCCCAAGCAGCAGTTTATTATTATCATCCTGGTGC | |
| | CCTATTTCTTTGTTAAAGTCAAAGAGCCACCTTTACCTTTTATTTTTAATGGTACA | |
| | TGGGACAGCTAAGGCTAAGAAGATTGAAGAAAGAAAATAATGAAGGTTTAAAAAAG | |
| | CCACATCTTTGATCCCTCACTGTCTACTTCTTCTTTCAGCAATATTCCTTTCACTG | |
| | TGGTTCATCCATGGGTCAAGATTCATTGATTCATTCACTCAAATCATTCATCTTAG | |
| | CAAAAACAATATATCACATAATCTGATGTTGAACTATAAAGGTTTCATCAGGTCAT | |
| | TCATTCACCCTGTCCACAAGCTGTGAATTATTATCTCTTTCCTGGTTGTATTTTGG | |
| | GATTACAATCATCTTGAGTCAAAGCTGGAAACTGAGTGGAAGTCTCTGGGAAAGAC | |
| | TCAAACCTCCTTAAGCTATACACCTCTTTTCCCCATCAGATTTTCCTTCCTTCAGT | |
| | TTCCACCAAAATGTGCTCTTGGATTTTTCATATGAATGTATAATGTACCTCAGGCC | |
| | TATAAGTATTTTAAAAGGGATCAAAATCTTAGTTTTAATGGAGGACATTTTTATGA | |
| | TGGACTCCTACAGCATCCATCAGAATATGTAAGATGATGAGGAATGTCTTCCTGTG | |
| | TTCCCAGATCTCATGCCACAGAGGCCCTTGCTTACTCTATGTTTGAATTGTATTTG | |
| | GAAAAAAAAAAAAAAACAAAAAACTAGGGCTAGCAAAATTGAAAAAAGATAAAAGA | |
| | CGAAAGAAGCCACATGTAAACATACTGTGTTTACTCTTCTAAAATATTAAAAAATG | |
| | AAAAGATCCAAAATCAAATTAATATTCCCCTGGAATTTCATATCTATTTCAGTGAC | |
| | TGTGGAGTGAATCTCACCACGAAAGTTGCTGCAGTCTTGTATAAGTTTCACATAGT | |
| | TTTACTGTGTTTGTGCCTATGTGAGAATAAACTACTGTGCATAAAATCTTGCTGTT | |
| | GAGCCATGTGTGAATTAGCTGTGTGATGTTACCTCCCTGTTACTACCAGGCTGGTT | |
| | TAGGATATCATTTCTGTATGTGGCACCAGGATTAGACCAATGACAGAAAAAGAAAG | |
| | TGCTCTCCCTGCCAAACTGGCCAATAAAACTGTTCCACATATCCCAGACTCAGGGT | |
| | TACCTAAACAACCTGTGTTTAAAGAGAACAAAAACAAAAGCCTCTGACATAGTCTT | |
| | ACTCCTTGCCAAATTCGTCAGAAAGCTGATGGATTCAAATTCCCCCAATATGAATC | |
| | CCGTATTTACATTATTTCTCTATTTTGACTACTTTTTTTTTTTTTTAAAGACTTTC | |
| | TAAATAGTTTCCCACTATCGAGGCTTCTTAGAGGAAACATTTCTCATTATTTCCCC | |
| | TTGGCTATTTGAAAAGGAATTTGTTCTTCCTTTTCCTCCATCTCTTAACACTACTA | |
| | CTACTAACAATAGTAACAACAATAGTAAGTACAGTAGGGTTTTTTGTTTTGTTTTT | |
| | AACTTAAGACATACTTTCTTGTTCTGGATACCAAAATATGTTTCACAGAGGCATCT | |
| | ACTTAGATGGGGTGCAGATGACACAGTTGTTAATTCTGGCAGGTACCTCTTGCTTC | |
| | TTCACTGCTGGGGCTACTCAGTGAGTGGCAGGAAGGTTGATTTGCTTTCCCCCCTT | |
| | TTCTTTTGCTCCTGGGCTCCTTCCCAGATGATGTGACGGGCCATGAAACAAAGACT | |
| | CTTTTCAGCTGTCGGTGTGCATAGAACTGGCTGCGGCTTCCTAGCTTGTCACATCT | |
| | CCGGTCTGAAGATGATCAAATAATGAGCAACACATCCAGGTTATAGGGAACACGGG | |
| | AAACACCCCGCAGCTGGGTGTACCCCAGCCCCTCAGAGTGCACATTGGTGTTGTTT | |
| | GTCCTAGTGGACTTCGGAGTAGGCCAGTGCCTTCTGGTCAGTTCCTCAGTGGCCCA | |
| | CATTCAGCTCTTAAAGGCAGAGCATGCTAACGGGAGGTCCAGGCTTCCGCCTGAGG | |
| | CCAAATACACCCCAAAAGCTCATCTGTTATAGCCTGATATGAAATCGGTTTCTTTC | |
| | TGCAACTGACCTGACTCATAGAAAGTGAAGCCTGGCTTTTCATAAGTGAAGTTTGG | |
| | CAGGCAAGGGAGGCAGGAAATCCAGAGGAGAATGAGCCTGTAAAGCATGGCTCCTT | |
| | CCAGCCCTTGTTACTTCCTCTGCCCAAGTGTGGGGGAGGGTCCTGTCTCTTGGCAT | |
| | CTGGGCCCAGCAAGAGTTCAGAGGTTTGGTAGTCTCTGCTTGGTCCATATGCAAAA | |
| | CACATGTATGTGTATACATTATTAATGGCAAGGGGGTTCCTGAAACTGAGAGGGAG | |
| | TAAGGAGACTTCTCATCTGCTCTTGGAAGAAGCAAGGAATGAAGCCAGTTCAGTAG | |
| | ACTGATTCCTGAGGCTTTGGGGCAGGAACTTTTTCTTTCTCCATATCCCCATGGAG | |
| | ATGGTTCATTTACCCTGAATTAAGATTTGGCCCTTCGGTGCAGTGCCAAGGCAGTT | |
| | TAAAGAGAAGAAAAGTAATTTCTGATCATTGACTAAGATCAAGGTAAATCATGACA | |
| | CTTATCCTTTCTATGATTTGCCAGTGACATGTTTTCTTAAGCCCAGAAATGATTTA | |
| | TTGATCGCAGCAGCCAGAATATATCACACTAAAACAGATCAGCCTGCCACTGTCTT | |
| | CTCAGGTCTCTCATGATTAAAGTGGCCTGCTTTAAAGTAGACTCAATGTGAATAGG | |
| | TCTCCATGACCTCTGCCTCACTGCGTAGCACTCACATCCTCACCCACTCTTGCACT | |
| | CTGGCTTCCCTGCGGTTCTTTCAATATGCCAGGCATGCTGGAACCCCGGAGCCTTT | |
| | GCACTGGCTGTTCCCTCTGTCTGTAACAGTCATTCGCAGAATCAACGCATGACTAA | |
| | TAGCCTCACTTCCATTGAGTCTTGACATTAGGAATGGATATACATGTCTATATTGG | |
| | GAAACCACAATAAAAATTGATGGTAGAGATGCAAATATGAGCAAGATAACAGGGTG | |
| | GGGGCAGGGGAGAGAGGTCAGTGGAGGACTTGGCACAGTAGCCTCTTAAATGGCAC | |
| | AATAGCCTCTTAAATTTTTGGTTAAGAAATCATTCACATTGATAAGTATGGCAGGA | |
| | TAAAGGTGTCCATGAGTGAATCCCGGGAACCTGTTACTTTATGTGGCAAAAGGGAC | |
| | TTTGCAGATGTGATTAACTTAAGGGGCTTGAGATGGGAAGATTTTTCCTGTTTTTA | |
| | TCAGTAGGCTTGATATAATTAAAAGGGTCCTTATAAGAGGGAAGCAAGAGTGTCAG | |
| | AGTCAGAGAAAGAGATGAAATGACAGATGTAGAGGTTGGAATGATGTGGTCAGGAA | |
| | CCAGGGAAAGCAGGGGGTATCTAGAAGCTGGAAAAGACAAGGGAATAGGGCTTCCC | |
| | CTAGATTCTCCAGAAATACAGCCCTATTGATATCTTGAGTTTAGTCCAGTGAGACT | |
| | TATTTTAGACTTCTGACATTTGGAACTGTAATATAATACCTTCATGTTATTTTTAT | |
| | TGCTGTTGTAACAAATAACCACAAACACAGTTCTACTAATTTCTTTCTCAAGGTAG | |
| | CTTCTCAATTTTGCCAACGCTGGTTACCATACATACTTAAAGTTTCATTTTGAGTC | |
| | TCTGAAAACTCACATCTCTCTTAATCTGCTCTACTTTTTTCTTGGCTTTGTATAGT | |
| | GCTTATGTTCTGCTACACTTTGTAATTTATTGATTATGCTTACCATGGGCAGGGAT | |
| | TCTTAACTGTTTTATTTATTTATATATCTTAAACATTGAAAACACTGGCATGTAGT | |
| | AGATGCTTAATAAGTAATTGTTGACTCAATCGATAAAATATACTAGAACATACAAG | |
| | ATTTTCCCAATGTAACATAACTAGTAAGAGGCTGAACCGGGATTTGAACTCAAAAT | |
| | TCATTCCCTGAACCTTCTTCTAGCAGCCACATTGAGGAAGAAATTACCAGGGCTGT | |
| | GTTCTCAACACAAGTGTTTTCCGAACCACAGAATTAAAGGCTGGTGGCCCATGTAT | |
| | CAGTGTCTGTATTTATGAGCCCCTCTTTCAATCTCTTTCTTTTCATATTGTGTTGA | |
| | TGCTGTAGCTTCTACTGGTCATGTTATTTTTTTGTTTCCCAAGACGGAATTATGTG | |
| | GCTTTATCTTTAATGTTGCATTATCAATACTTATAATAAATAATATTATGTATTAC | |
| | TCAATATTCATGATTAATAGTGTTACTATTGGTTATTTAATAATGTTTAACTTACA | |
| | TTAGCAGTTGTTACTATTTTTATGATGCTAAATTACTAACAGCTAAAACAACTTCT | |
| | ATATTAAAAAGTATATTTGAGTGCCACTCAAGAGATAATGAGTACCTTACAAAGAA | |
| | GAAATCTTGTTTCTCACCTTTGCGTCATTAAACAGATCAGGATTTGGAGAATTAAG | |
| | CCCTAAGTAATAGTGTTATTATTTTGATCTCACCCCTTTTTTTCTTATGAAATGGA | |
| | ATACTTTGGTTATCAGAAGCCACTTTAAGCATATATATATATATATATATATATAC | |
| | ATATATATATATATATATGTCATAATCCGAATAAAAATAGCATTCATGGAGGTTTC | |
| | TTTTGGAGCCTTTGGTAAAACACTCCATCGTGGGTCTCTGTCAAGATATCTGAAAA | |
| | CTTTTTCTTGGCTTCTGGCTTTGAACAAAGTTTCAGAGTAACAACAAGGCTTCATT | |
| | GTGCACTGAAATTTCTGTAAGGCAACATTCATTCAAGTGTTGATTCGCATTTCACC | |
| | ATCCAAGAATAACAACAGTTATTTATATAATTTTATCCACGTTTCTGTTTTTTCCT | |
| | ATCCATTTCACCCTTTCACCCCACCCCTGCTGAAACACTGGAGCTTGTTTGGGATG | |
| | GGGGtGGGGTGCCATGCAGACTACATACACATACAGATGTTTTTCTTTTTCTTTTC | |
| | CCGGTCTTGCTATGGGATAGACAGACTGGACTTTTTCTTATTAACAATATTATTTA | |
| | AAAGCTTGGAATTTATTATCATTTAATCATTTGTATGTAATGAAATAGGTCTCCAT | |
| | GGTAAAGATGTGTTTATTGACCAGCGGTTAGCTTTATTCAAATTAGGGTGACCATA | |
| | GAAGACCAAGGACTATGATATAATGTACAATCCTAAGTGGTTTGATTTAAATAAAA | |
| | AGAAAGACCAGGCATTTCAGCTAAAATCCCCACCAAAGCCCAATGACTAGATGGGC | |
| | ATCCATATGACTCAATGAAATTTTCTATGATCTTAAATGGCCATCTGAGTCCGTGA | |
| | AACTATAGGACTAACTATTCAATCCTTATTGAGAAAGCCTTGTTAATAGCTTGAAT | |
| | TGAGTTATATGGGATAGGAATGTTCATATCTTTATGACAATATATGCCACCTAAGC | |
| | TACATAACCAGCTGTGTTAGCTAAAATACTCTAAAGTGTAAAAAATCATAGTTTTC | |
| | TATTAAAGGAAGTCATGATTGTTAAAAATAATTTTTAAATAGTGTGCCTAGATTCT | |
| | TCTAGTATAATATATAATTTTTTTTTTTTTTTTATTTTGAGACAGAGTCTTGCTCT | |
| | GTCACCCAGGCTGGAGTGCAGTGGCTGGAGTTGCTCACTGCAACCTCGCCTCCCGG | |
| | GTTCAAGCGATTCTCGTGCCTCAGCCTCCCAAGTAGCTGAGATTACACGTGCCCAC | |
| | CACTATGTCCGGCTAATTTTTTTGAATTTTTAGTAGAGACTGGGTTTCATCATGCT | |
| | GGCCAGACTGGTCTTGAACTCCTGACCTCAGGTGATCTGCCCACCTCGGCCTCCCA | |
| | AAGTGCTGGGATTACAGGCATGAGCCATTGCGCCCAGCCGATATATAAATTTTTAT | |
| | ATGGCTCCATGATCTTCTCTACATTTAATGACAGAACTGGTGGAGGGGAAGAAAGA | |
| | GATGGGACTAAGCCAGAGATCAATATACATACAACTATACTTTGACCAAAAAAAGG | |
| | GAGATTGACTGGCAGGGGAATTAATAGTATGCAGAAGAGCAAGGTGAGTCCAGTCA | |
| | CTGTCATTATTCAAAAACAGCCTTTCAGGAGAAGTTTGCAACTGAATTTGGGACTG | |
| | TGGGCAGATAAGTCACAGGAATGATTCTATTGTGTATCCTGAAGTCATCCATCCAG | |
| | CTAGGAGTCAGAGGTGCAGGCTGAAAAGACATTGCCCCTAGAGTGGGGAACTGCCA | |
| | AAATCTAGCCAGGATATTAGGCCAAGAGAAAAGACCTCAGGCACAGGGGAAGCCAG | |
| | CTTCAGA | |
| 929 | AACGAACTCCATCTGGGATAGCAATAACCTGTGAAAATGCTCCCCCGGCTAATTTG | HAO1 exon 1 |
| | TATCAATGATTATGAACAACATGCTAAATCAGTACTTCCAAAGTCTATATATGACT | |
| | ATTACAGGTCTGGGGCAAATGATGAAGAAACTTTGGCTGATAATATTGCAGCATTT | |
| | TCCAGGTAAGAAAATTTATTTTTTAAAATCATGTTTTAAAATTACACAAAGACCG | |
| 930 | TGATTCTGAAACTCTAAAGCCTTTTATTTTATTTTATTTTTTAATTCTAGATGGAA | HAO1 exon 2 |
| | GCTGTATCCAAGGATGCTCCGGAATGTTGCTGAAACAGATCTGTCGACTTCTGTTT | |
| | TAGGACAGAGGGTCAGCATGCCAATATGTGTGGGGGCTACGGCCATGCAGCGCATG | |
| | GCTCATGTGGACGGCGAGCTTGCCACTGTGAGAGGTAGGAGGAAGATTGTCACCAC | |
| | AGGGACAGAAGGAGGCTAACGTTTATCG | |
| 931 | GGAGGGTAAGCAATGCCAGGCCCATCTTGATCATCCCCTTTCTTTCTCAGCCTGTC | HAO1 exon 3 |
| | AGTCCCTGGGAACGGGCATGATGTTGAGTTCCTGGGCCACCTCCTCAATTGAAGAA | |
| | GTGGCGGAAGCTGGTCCTGAGGCACTTCGTTGGCTGCAACTGTATATCTACAAGGA | |
| | CCGAGAAGTCACCAAGAAGCTAGTGCGGCAGGCAGAGAAGATGGGCTACAAGGCCA | |
| | TATTTGTGACAGTGGACACACCTTACCTGGGCAACCGTCTGGATGATGTGCGTAAC | |
| | AGATTCAAACTGCCGCCACAACTCAGGTAACCATGATCATGTGGGCCCCGAGCTGA | |
| | GGCGAAAGGGATCTTGACTG | |
| 932 | ACGTATTTCTAATTTGGCAAATTTCTCATTTTATGCATTTCTTATTTTAGGATGAA | HAO1 exon 4 |
| | AAATTTTGAAACCAGTACTTTATCATTTTCTCCTGAGGAAAATTTTGGAGACGACA | |
| | GTGGACTTGCTGCATATGTGGCTAAAGCAATAGACCCATCTATCAGCTGGGAAGAT | |
| | ATCAAATGGCTGAGAAGACTGACATCATTGCCAATTGTTGCAAAGGGCATTTTGAG | |
| | AGGTTCGTTTATTTCTCTACTTGAATTCATACTGACTTTGTGATCCTTTGTG | |
| 933 | CTGCCTGTTAAGTTACAGTTTCCCTAAGGTGCTTGTTTTACTCTCTCCAGGTGATG | HAO1 exon 5 |
| | ATGCCAGGGAGGCTGTTAAACATGGCTTGAATGGGATCTTGGTGTCGAATCATGGG | |
| | GCTCGACAACTCGATGGGGTGCCAGCCACTGTGAGTTTTGGCAGACGCTAAGATTT | |
| | CCTTTTGGAGTTCCCATTTCCATC | |
| 934 | TAACAATTCAGTGTTAATAGAGTCACATTATTGAACTTTTCTTTCCCCAGATTGAT | HAO1 exon 6 |
| | GTTCTGCCAGAAATTGTGGAGGCTGTGGAAGGGAAGGTGGAAGTCTTCCTGGACGG | |
| | GGGTGTGCGGAAAGGCACTGATGTTCTGAAAGCTCTGGCTCTTGGCGCCAAGGCTG | |
| | TGTTTGTGGGGAGACCAATCGTTTGGGGCTTAGCTTTCCAGGTAACTGGACAAAGA | |
| | AATGAATATATAAAATAGACAACTTGACAGTAAAACAAATGAATAAAACAAGTCAG | |
| | ACTGATTTAGTTCTGAATCACTCTGTATCTTTTCACTTGGTTAGGGGGAGAAAGGT | |
| | GTTCAAGATGTCCTCGAGATACTAAAGGAAGAATTCCGGTTGGCCATGGCTCTGAG | |
| | TGGTAAGACTCATTCTTGTTTACAACTTTCTTTTCTTTTATGATCTTTAAGT | |
| 935 | TGATTATTATTGCATTCAGTTCATATTAAATGTATGCATTATTTTTTCAGGGTGCC | HAO1 exon 7 |
| | AGAATGTGAAAGTCATCGACAAGACATTGGTGAGGAAAAATCCTTTGGCCGTTTCC | |
| | AAGATCTGACAGTGCACAATATTTTCCCATCTGTATTATTTTTTTTCAGCATGTAT | |
| | TACTTGACAAAGAGACACTGTGCAGAGGGTGACCACAGTCTGTAATTCCCCACTTC | |
| | AATACAAAGGGTGTCGTTCTTTTCCAACAAAATAGCAATCCCTTTTATTTCATTGC | |
| | TTTTGACTTTTCAATGGGTGTCCTAGGAACCTTTTAGAAAGAAATGGACTTTCATC | |
| | CTGGAAATATATTAACTGTTAAAAAGAAAACATTGAAAATGTGTTTAGACAACGTC | |
| | ATCCCCTGGCAGGCTAAAGTGCTGTATCCTTTAGTAAAATTGGAGGTAGCAAACAC | |
| | TAAGGTGAAAAGATAATGATCTCATTGTTTATTAACCTGTATTCTGTTTACATGTC | |
| | TTTAAAACAGTGGTTCTTAAATTGTAAGCTCAGGTTCAAAGTGTTGGTAATGCCTG | |
| | ATTCACAACTTTGAGAAGGTAGCACTGGAGAGAATTGGAATGGGTGGCGGTAATTG | |
| | GTGATACTTCTTTGAATGTAGATTTCCAATCACATCTTTAGTGTCTGAATATATCC | |
| | AAATGTTTTAGGATGTATGTTACTTCTTAGAGAGAAATAAAGCATTTTTGGGAAGA | |
| | A | |
| 965 | MSNKEKNASETRKAYTTKMIPRSHDRMKLLGNFMDYLMDGTPIFFELWNQFGGGID | (Cas12i1 of |
| | RDIISGTANKDKISDDLLLAVNWFKVMPINSKPQGVSPSNLANLFQQYSGSEPDIQ | SEQ ID NO: 3 |
| | AQEYFASNFDTEKHQWKDMRVEYERLLAELQLSRSDMHHDLKLMYKEKCIGLSLST | of U.S. Pat.No. |
| | AHYITSVMFGTGAKNNRQTKHQFYSKVIQLLEESTQINSVEQLASIILKAGDCDSY | 10,808,245) |
| | RKLRIRCSRKGATPSILKIVQDYELGTNHDDEVNVPSLIANLKEKLGRFEYECEWK | |
| | CMEKIKAFLASKVGPYYLGSYSAMLENALSPIKGMTTKNCKFVLKQIDAKNDIKYE | |
| | NEPFGKIVEGFFDSPYFESDTNVKWVLHPHHIGESNIKTLWEDLNAIHSKYEEDIA | |
| | SLSEDKKEKRIKVYQGDVCQTINTYCEEVGKEAKTPLVQLLRYLYSRKDDIAVDKI | |
| | IDGITFLSKKHKVEKQKINPVIQKYPSFNFGNNSKLLGKIISPKDKLKHNLKCNRN | |
| | QVDNYIWIEIKVLNTKTMRWEKHHYALSSTRFLEEVYYPATSENPPDALAARFRTK | |
| | TNGYEGKPALSAEQIEQIRSAPVGLRKVKKRQMRLEAARQQNLLPRYTWGKDFNIN | |
| | ICKRGNNFEVTLATKVKKKKEKNYKVVLGYDANIVRKNTYAAIEAHANGDGVIDYN | |
| | DLPVKPIESGFVTVESQVRDKSYDQLSYNGVKLLYCKPHVESRRSFLEKYRNGTMK | |
| | DNRGNNIQIDFMKDFEAIADDETSLYYFNMKYCKLLQSSIRNHSSQAKEYREEIFE | |
| | LLRDGKLSVLKLSSLSNLSFVMFKVAKSLIGTYFGHLLKKPKNSKSDVKAPPITDE | |
| | DKQKADPEMFALRLALEEKRLNKVKSKKEVIANKIVAKALELRDKYGPVLIKGENI | |
| | SDTTKKGKKSSTNSFLMDWLARGVANKVKEMVMMHQGLEFVEVNPNFTSHQDPFVH | |
| | KNPENTFRARYSRCTPSELTEKNRKEILSFLSDKPSKRPTNAYYNEGAMAFLATYG | |
| | LKKNDVLGVSLEKFKQIMANILHQRSEDQLLFPSRGGMFYLATYKLDADATSVNWN | |
| | GKQFWVCNADLVAAYNVGLVDIQKDFKKK | |
| 966 | MSISNNNILPYNPKLLPDDRKHKMLVDTFNQLDLIRNNLHDMIIALYGALKYDNIK | (Cas12i3 of |
| | QFASKEKPHISADALCSINWFRLVKINERKPAIESNQIISKFIQYSGHTPDKYALS | SEQ ID NO: |
| | HITGNHEPSHKWIDCREYAINYARIMHLSFSQFQDLATACLNCKILILNGTLTSSW | 14 of U.S. |
| | AWGANSALFGGSDKENFSVKAKILNSFIENLKDEMNTTKFQVVEKVCQQIGSSDAA | Pat. No. |
| | DLFDLYRSTVKDGNRGPATGRNPKVMNLFSQDGEISSEQREDFIESF | 10,808,245) |
| | QKVMQEKNSKQIIPHLDKLKYHLVKQSGLYDIYSWAAAIKNANSTIVASNSSNLNT | |
| | ILNKTEKQQTFEELRKDEKIVACSKILLSVNDTLPEDLHYNPSTSNLGKNLDVFFD | |
| | LLNENSVHTIENKEEKNKIVKECVNQYMEECKGLNKPPMPVLLTFISDYAHKHQAQ | |
| | DFLSAAKMNFIDLKIKSIKVVPTVHGSSPYTWISNLSKKNKDGKMIRTPNSSLIGW | |
| | IIPPEEIHDQKFAGQNPIIWAVLRVYCNNKWEMHHFPFSDSRFFTEVYAYKPNLPY | |
| | LPGGENRSKRFGYRHSTNLSNESRQILLDKSKYAKANKSVLRCMENMTHNVVFDPK | |
| | TSLNIRIKTDKNNSPVLDDKGRITFVMQINHRILEKYNNTKIEIGDRILAYDQNQS | |
| | ENHTYAILQRTEEGSHAHQFNGWYVRVLETGKVTSIVQGLSGPIDQLNYDGMPVTS | |
| | HKFNCWQADRSAFVSQFASLKISETETFDEAYQAINAQGAYTWNLFYLRILRKALR | |
| | VCHMENINQFREEILAISKNRLSPMSLGSLSQNSLKMIRAFKSIINCYMSRMSFVD | |
| | ELQKKEGDLELHTIMRLTDNKLNDKRVEKINRASSFLINKAHSMGCKMIVGESDLP | |
| | VADSKTSKKQNVDRMDWCARALSHKVEYACKLMGLAYRGIPAYMSSHQDPLVHLVE | |
| | SKRSVLRPRFVVADKSDVKQHHLDNLRRMLNSKTKVGTAVYYREAVELMCEELGIH | |
| | KTDMAKGKVSLSDFVDKFIGEKAIFPQRGGRFYMSTKRLTTGAKLICYSGSDVWLS | |
| | DADEIAAINIGMFVVCDQTGAFKKKKKEKLDDEECDILPFRPM | |
| 1024 | ATGCTCCCCCGGCTAATTTGTATCAATGATTATGAACAACATGCTAAATCAGTACT | HAO1 cDNA |
| | TCCAAAGTCTATATATGACTATTACAGGTCTGGGGCAAATGATGAAGAAACTTTGG | |
| | CTGATAATATTGCAGCATTTTCCAGATGGAAGCTGTATCCAAGGATGCTCCGGAAT | |
| | GTTGCTGAAACAGATCTGTCGACTTCTGTTTTAGGACAGAGGGTCAGCATGCCAAT | |
| | ATGTGTGGGGGCTACGGCCATGCAGCGCATGGCTCATGTGGACGGCGAGCTTGCCA | |
| | CTGTGAGAGCCTGTCAGTCCCTGGGAACGGGCATGATGTTGAGTTCCTGGGCCACC | |
| | TCCTCAATTGAAGAAGTGGCGGAAGCTGGTCCTGAGGCACTTCGTTGGCTGCAACT | |
| | GTATATCTACAAGGACCGAGAAGTCACCAAGAAGCTAGTGCGGCAGGCAGAGAAGA | |
| | TGGGCTACAAGGCCATATTTGTGACAGTGGACACACCTTACCTGGGCAACCGTCTG | |
| | GATGATGTGCGTAACAGATTCAAACTGCCGCCACAACTCAGGATGAAAAATTTTGA | |
| | AACCAGTACTTTATCATTTTCTCCTGAGGAAAATTTTGGAGACGACAGTGGACTTG | |
| | CTGCATATGTGGCTAAAGCAATAGACCCATCTATCAGCTGGGAAGATATCAAATGG | |
| | CTGAGAAGACTGACATCATTGCCAATTGTTGCAAAGGGCATTTTGAGAGGTGATGA | |
| | TGCCAGGGAGGCTGTTAAACATGGCTTGAATGGGATCTTGGTGTCGAATCATGGGG | |
| | CTCGACAACTCGATGGGGTGCCAGCCACTATTGATGTTCTGCCAGAAATTGTGGAG | |
| | GCTGTGGAAGGGAAGGTGGAAGTCTTCCTGGACGGGGGTGTGCGGAAAGGCACTGA | |
| | TGTTCTGAAAGCTCTGGCTCTTGGCGCCAAGGCTGTGTTTGTGGGGAGACCAATCG | |
| | TTTGGGGCTTAGCTTTCCAGGGGGAGAAAGGTGTTCAAGATGTCCTCGAGATACTA | |
| | AAGGAAGAATTCCGGTTGGCCATGGCTCTGAGTGGGTGCCAGAATGTGAAAGTCAT | |
| | CGACAAGACATTGGTGAGGAAAAATCCTTTGGCCGTTTCCAAGATCTGA | |
| 1082 | rArGrArArArUrCrCrGrUrCrUrUrUrCrArUrUrGrArCrGrGrCrGrGrArG | 3′ end |
| | rCrArUrCrCrUrUrGrGrArUmA*mC*mA*rG | modified RNA |
| | | guide |
| | | targeting |
| | | HAO1 |
| | | sequence of |
| | | SEQ ID NO: |
| | | 1047 |
| 1083 | mA*mG*mA*rArArUrCrCrGrUrCrUrUrUrCrArUrUrGrArCrGrGrCrGrGr | 5′ and 3′ end |
| | ArGrCrArUrCrCrUrUrGrGrArUrmA*mC*mA*rG | modified RNA |
| | | guide |
| | | targeting |
| | | HAO1 |
| | | sequence of |
| | | SEQ ID NO: |
| | | 1047 |
| 1084 | rArGrArArArUrCrCrGrUrCrUrUrUrCrArUrUrGrArCrGrGrGrGrArArG | 3′ end |
| | rUrArCrUrGrArUrUrUrArGmC*mA*mU*rG | modified RNA |
| | | guide |
| | | targeting |
| | | HAO1 |
| | | sequence of |
| | | SEQ ID NO: |
| | | 1026 |
| 1085 | mA*mG*mA*rArArUrCrCrGrUrCrUrUrUrCrArUrUrGrArCrGrGrGrGrAr | 5′ and 3′ end |
| | ArGrUrArCrUrGrArUrUrUrArGmC*mA*mU*rG | modified RNA |
| | | guide |
| | | targeting |
| | | HAO1 |
| | | sequence of |
| | | SEQ ID NO: |
| | | 1026 |
| 1086 | rArGrArArArUrCrCrGrUrCrUrUrUrCrArUrUrGrArCrGrGrCrArArArG | 3′ end |
| | rUrCrUrArUrArUrArUrGrAmC*mU*mA*rU | modified RNA |
| | | guide |
| | | targeting |
| | | HAO1 |
| | | sequence of |
| 1087 | mA*mG*mA*rArArUrCrCrGrUrCrUrUrUrCrArUrUrGrArCrGrGrCrArAr | SEQ ID NO: |
| | ArGrUrCrUrArUrArUrArUrGrAmC*mU*mA*rU | 1025 |
| | | 5′ and 3′ end |
| | | modified RNA |
| | | guide |
| | | targeting |
| | | HAO1 |
| | | sequence of |
| | | SEQ ID NO: |
| | | 1025 |

In some embodiments, the gene editing system disclosed herein may comprise a Cas12i polypeptide as disclosed herein. In other embodiments, the gene editing system may comprise a nucleic acid encoding the Cas12i polypeptide. For example, the gene editing system may comprise a vector (e.g., a viral vector such as an AAV vector, such as AAV1, AAV2, AAV3, AAV4, AAV5, AAV6, AAV7, AAV8, AAV9, AAVrh10, AAV11 and AAV12) encoding the Cas12i polypeptide. Alternatively, the gene editing system may comprise a mRNA molecule encoding the Cas12i polypeptide. In some instances, the mRNA molecule may be codon-optimized.

### II. Preparation of Gene Editing System Components

The present disclosure provides methods for production of components of the gene editing systems disclosed herein, e.g., the RNA guide, methods for production of the Cas12i polypeptide, and methods for complexing the RNA guide and Cas12i polypeptide.

### A. RNA Guide

In some embodiments, the RNA guide is made by in vitro transcription of a DNA template. Thus, for example, in some embodiments, the RNA guide is generated by in vitro transcription of a DNA template encoding the RNA guide using an upstream promoter sequence (e.g., a T7 polymerase promoter sequence). In some embodiments, the DNA template encodes multiple RNA guides or the in vitro transcription reaction includes multiple different DNA templates, each encoding a different RNA guide. In some embodiments, the RNA guide is made using chemical synthetic methods. In some embodiments, the RNA guide is made by expressing the RNA guide sequence in cells transfected with a plasmid including sequences that encode the RNA guide. In some embodiments, the plasmid encodes multiple different RNA guides. In some embodiments, multiple different plasmids, each encoding a different RNA guide, are transfected into the cells. In some embodiments, the RNA guide is expressed from a plasmid that encodes the RNA guide and also encodes a Cas12i polypeptide. In some embodiments, the RNA guide is expressed from a plasmid that expresses the RNA guide but not a Cas12i polypeptide. In some embodiments, the RNA guide is purchased from a commercial vendor. In some embodiments, the RNA guide is synthesized using one or more modified nucleotide, e.g., as described above.

### B. Cas12i Polypeptide

In some embodiments, the Cas12i polypeptide of the present disclosure can be prepared by (a) culturing bacteria which produce the Cas12i polypeptide of the present disclosure, isolating the Cas12i polypeptide, optionally, purifying the Cas12i polypeptide, and complexing the Cas12i polypeptide with an RNA guide. The Cas12i polypeptide can be also prepared by (b) a known genetic engineering technique, specifically, by isolating a gene encoding the Cas12i polypeptide of the present disclosure from bacteria, constructing a recombinant expression vector, and then transferring the vector into an appropriate host cell that expresses the RNA guide for expression of a recombinant protein that complexes with the RNA guide in the host cell. Alternatively, the Cas12i polypeptide can be prepared by (c) an in vitro coupled transcription-translation system and then complexing with an RNA guide.

In some embodiments, a host cell is used to express the Cas12i polypeptide. The host cell is not particularly limited, and various known cells can be preferably used. Specific examples of the host cell include bacteria such as E. coli, yeasts (budding yeast, Saccharomyces cerevisiae, and fission yeast, Schizosaccharomyces pombe), nematodes (Caenorhabditis elegans), Xenopus laevis oocytes, and animal cells (for example, CHO cells, COS cells and HEK293 cells). The method for transferring the expression vector described above into host cells, i.e., the transformation method, is not particularly limited, and known methods such as electroporation, the calcium phosphate method, the liposome method and the DEAE dextran method can be used.

After a host is transformed with the expression vector, the host cells may be cultured, cultivated or bred, for production of the Cas12i polypeptide. After expression of the Cas12i polypeptide, the host cells can be collected and Cas12i polypeptide purified from the cultures etc. according to conventional methods (for example, filtration, centrifugation, cell disruption, gel filtration chromatography, ion exchange chromatography, etc.).

In some embodiments, the methods for Cas12i polypeptide expression comprises translation of at least 5 amino acids, at least 10 amino acids, at least 15 amino acids, at least 20 amino acids, at least 50 amino acids, at least 100 amino acids, at least 150 amino acids, at least 200 amino acids, at least 250 amino acids, at least 300 amino acids, at least 400 amino acids, at least 500 amino acids, at least 600 amino acids, at least 700 amino acids, at least 800 amino acids, at least 900 amino acids, or at least 1000 amino acids of the Cas12i polypeptide. In some embodiments, the methods for protein expression comprises translation of about 5 amino acids, about 10 amino acids, about 15 amino acids, about 20 amino acids, about 50 amino acids, about 100 amino acids, about 150 amino acids, about 200 amino acids, about 250 amino acids, about 300 amino acids, about 400 amino acids, about 500 amino acids, about 600 amino acids, about 700 amino acids, about 800 amino acids, about 900 amino acids, about 1000 amino acids or more of the Cas12i polypeptide.

A variety of methods can be used to determine the level of production of a Cas12i polypeptide in a host cell. Such methods include, but are not limited to, for example, methods that utilize either polyclonal or monoclonal antibodies specific for the Cas12i polypeptide or a labeling tag as described elsewhere herein. Exemplary methods include, but are not limited to, enzyme-linked immunosorbent assays (ELISA), radioimmunoassays (MA), fluorescent immunoassays (FIA), and fluorescent activated cell sorting (FACS). These and other assays are well known in the art (Sec, e.g., Maddox et al., J. Exp. Med. 158:1211 [1983]).

The present disclosure provides methods of in vivo expression of the Cas12i polypeptide in a cell, comprising providing a polyribonucleotide encoding the Cas12i polypeptide to a host cell wherein the polyribonucleotide encodes the Cas12i polypeptide, expressing the Cas12i polypeptide in the cell, and obtaining the Cas12i polypeptide from the cell.

The present disclosure further provides methods of in vivo expression of a Cas12i polypeptide in a cell, comprising providing a polyribonucleotide encoding the Cas12i polypeptide to a host cell wherein the polyribonucleotide encodes the Cas12i polypeptide and expressing the Cas12i polypeptide in the cell. In some embodiments, the polyribonucleotide encoding the Cas12i polypeptide is delivered to the cell with an RNA guide and, once expressed in the cell, the Cas12i polypeptide and the RNA guide form a complex. In some embodiments, the polyribonucleotide encoding the Cas12i polypeptide and the RNA guide are delivered to the cell within a single composition. In some embodiments, the polyribonucleotide encoding the Cas12i polypeptide and the RNA guide are comprised within separate compositions. In some embodiments, the host cell is present in a subject, e.g., a human patient.

### C. Complexes

In some embodiments, an RNA guide targeting HAO1 is complexed with a Cas12i polypeptide to form a ribonucleoprotein. In some embodiments, complexation of the RNA guide and Cas12i polypeptide occurs at a temperature lower than about any one of 20° C., 21° ° C., 22° C., 23° C. 24° C., 25° C., 26° ° C., 27° ° C., 28° ° C., 29° C., 30° ° C., 31° C. 32° C. 33° C., 34° C., 35° C., 36° C., 37° ° C., 38° C., 39° C., 40° ° C. 41° C., 42° ° C., 43° C., 44° C., 45° C., 50° C., or 55° ° C. In some embodiments, the RNA guide does not dissociate from the Cas12i polypeptide at about 37° C. over an incubation period of at least about any one of 10 mins, 15 mins, 20 mins, 25 mins, 30 mins, 35 mins, 40 mins, 45 mins, 50 mins, 55 mins, 1 hr, 2 hr, 3 hr, 4 hr, or more hours.

In some embodiments, the RNA guide and Cas12i polypeptide are complexed in a complexation buffer. In some embodiments, the Cas12i polypeptide is stored in a buffer that is replaced with a complexation buffer to form a complex with the RNA guide. In some embodiments, the Cas12i polypeptide is stored in a complexation buffer.

In some embodiments, the complexation buffer has a pH in a range of about 7.3 to 8.6. In one embodiment, the pH of the complexation buffer is about 7.3. In one embodiment, the pH of the complexation buffer is about 7.4. In one embodiment, the pH of the complexation buffer is about 7.5. In one embodiment, the pH of the complexation buffer is about 7.6. In one embodiment, the pH of the complexation buffer is about 7.7. In one embodiment, the pH of the complexation buffer is about 7.8. In one embodiment, the pH of the complexation buffer is about 7.9. In one embodiment, the pH of the complexation buffer is about 8.0. In one embodiment, the pH of the complexation buffer is about 8.1. In one embodiment, the pH of the complexation buffer is about 8.2. In one embodiment, the pH of the complexation buffer is about 8.3. In one embodiment, the pH of the complexation buffer is about 8.4. In one embodiment, the pH of the complexation buffer is about 8.5. In one embodiment, the pH of the complexation buffer is about 8.6.

In some embodiments, the Cas12i polypeptide can be overexpressed and complexed with the RNA guide in a host cell prior to purification as described herein. In some embodiments, mRNA or DNA encoding the Cas12i polypeptide is introduced into a cell so that the Cas12i polypeptide is expressed in the cell. In some embodiments, the RNA guide is also introduced into the cell, whether simultaneously, separately, or sequentially from a single mRNA or DNA construct, such that the ribonucleoprotein complex is formed in the cell.

### III. Genetic Editing Methods

The disclosure also provides methods of modifying a target site within the HAO1 gene. In some embodiments, the methods comprise introducing an HAO1-targeting RNA guide and a Cas12i polypeptide into a cell. The HAO1-targeting RNA guide and Cas12i polypeptide can be introduced as a ribonucleoprotein complex into a cell. The HAO1-targeting RNA guide and Cas12i polypeptide can be introduced on a nucleic acid vector. The Cas12i polypeptide can be introduced as an mRNA. The RNA guide can be introduced directly into the cell. In some embodiments, the composition described herein is delivered to a cell/tissue/liver/person to reduce HAO1 in the cell/tissue/liver/person. In some embodiments, the composition described herein is delivered to a cell/tissue/liver/person to reduce oxalate production in the cell/tissue/liver/person. In some embodiments, the composition described herein is delivered to a cell/tissue/liver/person to correct calcium oxalate crystal deposition in the cell/tissue/liver/person. In some embodiments, the composition described herein is delivered to a person with primary hyperoxaluria.

Any of the gene editing systems disclosed herein may be used to genetically engineered an HAO1 gene. The gene editing system may comprise an RNA guide and a Cas12i2 polypeptide. The RNA guide comprises a spacer sequence specific to a target sequence in the HAO1 gene, e.g., specific to a region in exon1 or exon 2 of the HAO1 gene.

### A. Target Sequence

In some embodiments, an RNA guide as disclosed herein is designed to be complementary to a target sequence that is adjacent to a 5′-TTN-3′ PAM sequence or 5′-NTTN-3′ PAM sequence.

In some embodiments, the target sequence is within an HAO1 gene or a locus of an HAO1 gene (e.g., in exon1 or exon 2), to which the RNA guide can bind via base pairing. In some embodiments, a cell has only one copy of the target sequence. In some embodiments, a cell has more than one copy, such as at least about any one of 2, 3, 4, 5, 10, 100, or more copies of the target sequence.

In some embodiments, the HAO1 gene is a mammalian gene. In some embodiments, the HAO1 gene is a human gene. For example, in some embodiments, the target sequence is within the sequence of SEQ ID NO: 928 (or the reverse complement thereof). In some embodiments, the target sequence is within an exon of the HAO1 gene set forth in SEQ ID NO: 928, e.g., within a sequence of SEQ ID NO: 929, 930, 931, 932, 933, 934, or 935 (or a reverse complement thereof). Target sequences within an exon region of the HAO1 gene of SEQ ID NO: 928 are set forth in Table 5. In some embodiments, the target sequence is within an intron of the HAO1 gene set forth in SEQ ID NO: 928 (or the reverse complement thereof). In some embodiments, the target sequence is within a variant (e.g., a polymorphic variant) of the HAO1 gene sequence set forth in SEQ ID NO: 928 (or the reverse complement thereof). In some embodiments, the HAO1 gene sequence is a homolog of the sequence set forth in SEQ ID NO: 928 (or the reverse complement thereof). For examples, in some embodiments, the HAO1 gene sequence is a non-human HAO1 sequence. In some embodiments, the HAO1 gene sequence is a coding sequence set forth in SEQ ID NO: 1024 (or the reverse complement thereof). In some embodiments, the HAO1 gene sequence is a homolog of a coding sequence set forth in SEQ ID NO: 1024 (or the reverse complement thereof).

In some embodiments, the target sequence is adjacent to a 5′-TTN-3′ PAM sequence or a 5′-NTTN-3′ PAM sequence, wherein N is any nucleotide. The 5′-NTTN-3′ sequence may be immediately adjacent to the target sequence or, for example, within a small number (e.g., 1, 2, 3, 4, or 5) of nucleotides of the target sequence. In some embodiments the 5′-NTTN-3′ sequence is 5′-NTTY-3′, 5′-NTTC-3′, 5′-NTTT-3′, 5′-NTTA-3′, 5′-NTTB-3′, 5′-NTTG-3′, 5′-CTTY-3′, 5′-DTTR-3′, 5′-CTTR-3′, 5′-DTTT-3′, 5′-ATTN-3′, or 5′-GTTN-3′, wherein Y is C or T. B is any nucleotide except for A, D is any nucleotide except for C, and R is A or G. In some embodiments, the 5′-NTTN-3′ sequence is 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′. The PAM sequence may be 5′ to the target sequence.

The 5′-NTTN-3′ sequence may be immediately adjacent to the target sequence or, for example, within a small number (e.g., 1, 2, 3, 4, or 5) of nucleotides of the target sequence. In some embodiments the 5′-NTTN-3′ sequence is 5′-NTTY-3′, 5′-NTTC-3′, 5′-NTTT-3′, 5′-NTTA-3′, 5′-NTTB-3′, 5′-NTTG-3′, 5′-CTTY-3′, 5′-DTTR-3′, 5′-CTTR-3′, 5′-DTTT-3′, 5′-ATTN-3′, or 5′-GTTN-3′, wherein Y is C or T. B is any nucleotide except for A, D is any nucleotide except for C, and R is A or G. In some embodiments, the 5′-NTTN-3′ sequence is 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′. In some embodiments, the RNA guide is designed to bind to a first strand of a double-stranded target nucleic acid (i.e., the non-PAM strand), and the 5′-NTTN-3′ PAM sequence is present in the second, complementary strand (i.e., the PAM strand). In some embodiments, the RNA guide binds to a region on the non-PAM strand that is complementary to a target sequence on the PAM strand, which is adjacent to a 5′-NAAN-3′ sequence.

In some embodiments, the target sequence is present in a cell. In some embodiments, the target sequence is present in the nucleus of the cell. In some embodiments, the target sequence is endogenous to the cell. In some embodiments, the target sequence is a genomic DNA. In some embodiments, the target sequence is a chromosomal DNA. In some embodiments, the target sequence is a protein-coding gene or a functional region thereof, such as a coding region, or a regulatory element, such as a promoter, enhancer, a 5′ or 3′ untranslated region, etc.

In some embodiments, the target sequence is present in a readily accessible region of the target sequence. In some embodiments, the target sequence is in an exon of a target gene. In some embodiments, the target sequence is across an exon-intron junction of a target gene. In some embodiments, the target sequence is present in a non-coding region, such as a regulatory region of a gene.

### B. Gene Editing

In some embodiments, the Cas12i polypeptide has enzymatic activity (e.g., nuclease activity). In some embodiments, the Cas12i polypeptide induces one or more DNA double-stranded breaks in the cell. In some embodiments, the Cas12i polypeptide induces one or more DNA single-stranded breaks in the cell. In some embodiments, the Cas12i polypeptide induces one or more DNA nicks in the cell. In some embodiments, DNA breaks and/or nicks result in formation of one or more indels (e.g., one or more deletions).

In some embodiments, an RNA guide disclosed herein forms a complex with the Cas12i polypeptide and directs the Cas12i polypeptide to a target sequence adjacent to a 5′-NTTN-3′ sequence. In some embodiments, the complex induces a deletion (e.g., a nucleotide deletion or DNA deletion) adjacent to the 5′-NTTN-3′ sequence. In some embodiments, the complex induces a deletion adjacent to a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the complex induces a deletion adjacent to a T/C-rich sequence.

In some embodiments, the deletion is downstream of a 5′-NTTN-3′ sequence. In some embodiments, the deletion is downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion is downstream of a T/C-rich sequence.

In some embodiments, the deletion alters expression of the HAO1 gene. In some embodiments, the deletion alters function of the HAO1 gene. In some embodiments, the deletion inactivates the HAO1 gene. In some embodiments, the deletion is a frameshifting deletion. In some embodiments, the deletion is a non-frameshifting deletion. In some embodiments, the deletion leads to cell toxicity or cell death (e.g., apoptosis).

In some embodiments, the deletion starts within about 5 to about 15 nucleotides (e.g., about 3, 4, 5, 6, 7, 8, 9, 10, 11, 12, 13, 14, 15, 16, or 17 nucleotides) of the 5′-NTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′. 5′-CTTA-3′, 5′-CTTT-3′, 5′-CTTG-3′, or 5′-CTTC-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) of a T/C-rich sequence.

In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of the 5′-NTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of a T/C-rich sequence.

In some embodiments, the deletion starts within about 5 to about 10 nucleotides (e.g., about 3, 4, 5, 6, 7, 8, 9, 10, 11, or 12 nucleotides) of the 5′-NTTN-3′ sequence. In some embodiments, the deletion starts within about 5 to about 10 nucleotides (e.g., about 3, 4, 5, 6, 7, 8, 9, 10, 11, or 12 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 10 nucleotides (e.g., about 3, 4, 5, 6, 7, 8, 9, 10, 11, or 12 nucleotides) of a T/C-rich sequence.

In some embodiments, the deletion starts within about 5 to about 10 nucleotides (e.g., about 3, 4, 5, 6, 7, 8, 9, 10, 11, or 12 nucleotides) downstream of the 5′-NTTN-3′ sequence. In some embodiments, the deletion starts within about 5 to about 10 nucleotides (e.g., about 3, 4, 5, 6, 7, 8, 9, 10, 11, or 12 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 10 nucleotides (e.g., about 3, 4, 5, 6, 7, 8, 9, 10, 11, or 12 nucleotides) downstream of a T/C-rich sequence.

In some embodiments, the deletion starts within about 10 to about 15 nucleotides (e.g., about 8, 9, 10, 11, 12, 13, 14, 15, 16, or 17 nucleotides) of the 5′-NTTN-3′ sequence. In some embodiments, the deletion starts within about 10 to about 15 nucleotides (e.g., about 8, 9, 10, 11, 12, 13, 14, 15, 16, or 17 nucleotides) of a 5′-ATTA-3′. 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 10 to about 15 nucleotides (e.g., about 8, 9, 10, 11, 12, 13, 14, 15, 16, or 17 nucleotides) of a T/C-rich sequence.

In some embodiments, the deletion starts within about 10 to about 15 nucleotides (e.g., about 8, 9, 10, 11, 12, 13, 14, 15, 16, or 17 nucleotides) downstream of the 5′-NTTN-3′ sequence. In some embodiments, the deletion starts within about 10 to about 15 nucleotides (e.g., about 8, 9, 10, 11, 12, 13, 14, 15, 16, or 17 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 10 to about 15 nucleotides (e.g., about 8, 9, 10, 11, 12, 13, 14, 15, 16, or 17 nucleotides) downstream of a T/C-rich sequence.

In some embodiments, the deletion ends within about 20 to about 30 nucleotides (e.g., about 17, 18, 19, 20, 21, 22, 23, 24, 25, 26, 27, 28, 29, 30, 31, 32, or 33 nucleotides) of the 5′-NTTN-3′ sequence. In some embodiments, the deletion ends within about 20 to about 30 nucleotides (e.g., about 17, 18, 19, 20, 21, 22, 23, 24, 25, 26, 27, 28, 29, 30, 31, 32, or 33 nucleotides) of a 5′-ATTA-3′, 5′-ATTT-3′, 5′-ATTG-3′, 5′-ATTC-3′, 5′-TTTA-3′, 5′-TTTT-3′. 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion ends within about 20 to about 30 nucleotides (e.g., about 17, 18, 19, 20, 21, 22, 23, 24, 25, 26, 27, 28, 29, 30, 31, 32, or 33 nucleotides) of a T/C-rich sequence.

In some embodiments, the deletion ends within about 20 to about 30 nucleotides (e.g., about 17, 18, 19, 20, 21, 22, 23, 24, 25, 26, 27, 28, 29, 30, 31, 32, or 33 nucleotides) downstream of the 5′-NTTN-3′ sequence. In some embodiments, the deletion ends within about 20 to about 30 nucleotides (e.g., about 17, 18, 19, 20, 21, 22, 23, 24, 25, 26, 27, 28, 29, 30, 31, 32, or 33 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′. 5′-CTTT-3′, 5′-CTTG-3′, or 5′-CTTC-3′ sequence. In some embodiments, the deletion ends within about 20 to about 30 nucleotides (e.g., about 17, 18, 19, 20, 21, 22, 23, 24, 25, 26, 27, 28, 29, 30, 31, 32, or 33 nucleotides) downstream of a T/C-rich sequence.

In some embodiments, the deletion ends within about 20 to about 25 nucleotides (e.g., about 17, 18, 19, 20, 21, 22, 23, 24, 25, 26, 27, or 28 nucleotides) of the 5′-NTTN-3′ sequence. In some embodiments, the deletion ends within about 20 to about 25 nucleotides (e.g., about 17, 18, 19, 20, 21, 22, 23, 24, 25, 26, 27, or 28 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion ends within about 20 to about 25 nucleotides (e.g., about 17, 18, 19, 20, 21, 22, 23, 24, 25, 26, 27, or 28 nucleotides) of a T/C-rich sequence. In some embodiments, the deletion ends within about 20 to about 25 nucleotides (e.g., about 17, 18, 19, 20, 21, 22, 23, 24, 25, 26, 27, or 28 nucleotides) downstream of the 5′-NTTN-3′ sequence. In some embodiments, the deletion ends within about 20 to about 25 nucleotides (e.g., about 17, 18, 19, 20, 21, 22, 23, 24, 25, 26, 27, or 28 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion ends within about 20 to about 25 nucleotides (e.g., about 17, 18, 19, 20, 21, 22, 23, 24, 25, 26, 27, or 28 nucleotides) downstream of a T/C-rich sequence.

In some embodiments, the deletion ends within about 25 to about 30 nucleotides (e.g., about 22, 23, 24, 25, 26, 27, 28, 29, 30, 31, 32, or 33 nucleotides) of the 5′-NTTN-3′ sequence. In some embodiments, the deletion ends within about 25 to about 30 nucleotides (e.g., about 22, 23, 24, 25, 26, 27, 28, 29, 30, 31, 32, or 33 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′. 5′-CTTT-3′, 5′-CTTG-3′, or 5′-CTTC-3′ sequence. In some embodiments, the deletion ends within about 25 to about 30 nucleotides (e.g., about 22, 23, 24, 25, 26, 27, 28, 29, 30, 31, 32, or 33 nucleotides) of a T/C-rich sequence.

In some embodiments, the deletion ends within about 25 to about 30 nucleotides (e.g., about 22, 23, 24, 25, 26, 27, 28, 29, 30, 31, 32, or 33 nucleotides) downstream of the 5′-NTTN-3′ sequence. In some embodiments, the deletion ends within about 25 to about 30 nucleotides (e.g., about 22, 23, 24, 25, 26, 27, 28, 29, 30, 31, 32, or 33 nucleotides) downstream of a 5′-ATTA-3′. 5′-ATTT-3′, 5′-ATTG-3′. 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion ends within about 25 to about 30 nucleotides (e.g., about 22, 23, 24, 25, 26, 27, 28, 29, 30, 31, 32, or 33 nucleotides) downstream of a T/C-rich sequence.

In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of the 5′-NTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of a T/C-rich sequence.

In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of the 5′-NTTN-3′ sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the 5′-NTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of a 5′-ATTA-3′. 5′-ATTT-3′. 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of a T/C-rich sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the T/C-rich sequence.

In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 20 to about 25 nucleotides (e.g., about 17, 18, 19, 20, 21, 22, 23, 24, 25, 26, 27, or 28 nucleotides) of the 5′-NTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 20 to about 25 nucleotides (e.g., about 17, 18, 19, 20, 21, 22, 23, 24, 25, 26, 27, or 28 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 20 to about 25 nucleotides (e.g., about 17, 18, 19, 20, 21, 22, 23, 24, 25, 26, 27, or 28 nucleotides) of a T/C-rich sequence.

In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of the 5′-NTTN-3′ sequence and ends within about 20 to about 25 nucleotides (e.g., about 17, 18, 19, 20, 21, 22, 23, 24, 25, 26, 27, or 28 nucleotides) downstream of the 5′-NTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of a 5′-ATTA-3′. 5′-ATTT-3′, 5′-ATTG-3′, 5′-ATTC-3′. 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence and ends within about 20 to about 25 nucleotides (e.g., about 17, 18, 19, 20, 21, 22, 23, 24, 25, 26, 27, or 28 nucleotides) downstream of the 5′-ATTA-3′, 5′-ATTT-3′, 5′-ATTG-3′, 5′-ATTC-3′. 5′-TTTA-3′. 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of a T/C-rich sequence and ends within about 20 to about 25 nucleotides (e.g., about 17, 18, 19, 20, 21, 22, 23, 24, 25, 26, 27, or 28 nucleotides) downstream of the T/C-rich sequence.

In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 25 to about 30 nucleotides (e.g., about 22, 23, 24, 25, 26, 27, 28, 29, 30, 31, 32, or 33 nucleotides) of the 5′-NTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 25 to about 30 nucleotides (e.g., about 22, 23, 24, 25, 26, 27, 28, 29, 30, 31, 32, or 33 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 25 to about 30 nucleotides (e.g., about 22, 23, 24, 25, 26, 27, 28, 29, 30, 31, 32, or 33 nucleotides) of a T/C-rich sequence.

In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of the 5′-NTTN-3′ sequence and ends within about 25 to about 30 nucleotides (e.g., about 22, 23, 24, 25, 26, 27, 28, 29, 30, 31, 32, or 33 nucleotides) downstream of the 5′-NTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence and ends within about 25 to about 30 nucleotides (e.g., about 22, 23, 24, 25, 26, 27, 28, 29, 30, 31, 32, or 33 nucleotides) downstream of the 5′-ATTA-3′, 5′-ATTT-3′. 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of a T/C-rich sequence and ends within about 25 to about 30 nucleotides (e.g., about 22, 23, 24, 25, 26, 27, 28, 29, 30, 31, 32, or 33 nucleotides) downstream of the T/C-rich sequence.

In some embodiments, the deletion starts within about 5 to about 10 nucleotides (e.g., about 3, 4, 5, 6, 7, 8, 9, 10, 11, or 12 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of the 5′-NTTN-3′ sequence. In some embodiments, the deletion starts within about 5 to about 10 nucleotides (e.g., about 3, 4, 5, 6, 7, 8, 9, 10, 11, or 12 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of a 5′-ATTA-3′, 5′-ATTT-3′, 5′-ATTG-3′, 5′-ATTC-3′, 5′-TTTA-3′, 5′-TTTT-3′. 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 10 nucleotides (e.g., about 3, 4, 5, 6, 7, 8, 9, 10, 11, or 12 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of a T/C-rich sequence.

In some embodiments, the deletion starts within about 5 to about 10 nucleotides (e.g., about 3, 4, 5, 6, 7, 8, 9, 10, 11, or 12 nucleotides) downstream of the 5′-NTTN-3′ sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the 5′-NTTN-3′ sequence. In some embodiments, the deletion starts within about 5 to about 10 nucleotides (e.g., about 3, 4, 5, 6, 7, 8, 9, 10, 11, or 12 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 10 nucleotides (e.g., about 3, 4, 5, 6, 7, 8, 9, 10, 11, or 12 nucleotides) downstream of a T/C-rich sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the T/C-rich sequence.

In some embodiments, the deletion starts within about 5 to about 10 nucleotides (e.g., about 3, 4, 5, 6, 7, 8, 9, 10, 11, or 12 nucleotides) and ends within about 20 to about 25 nucleotides (e.g., about 17, 18, 19, 20, 21, 22, 23, 24, 25, 26, 27, or 28 nucleotides) of the 5′-NTTN-3′ sequence. In some embodiments, the deletion starts within about 5 to about 10 nucleotides (e.g., about 3, 4, 5, 6, 7, 8, 9, 10, 11, or 12 nucleotides) and ends within about 20 to about 25 nucleotides (e.g., about 17, 18, 19, 20, 21, 22, 23, 24, 25, 26, 27, or 28 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 10 nucleotides and ends within about 20 to about 25 nucleotides (e.g., about 17, 18, 19, 20, 21, 22, 23, 24, 25, 26, 27, or 28 nucleotides) (e.g., about 3, 4, 5, 6, 7, 8, 9, 10, 11, or 12 nucleotides) of a T/C-rich sequence.

In some embodiments, the deletion starts within about 5 to about 10 nucleotides (e.g., about 3, 4, 5, 6, 7, 8, 9, 10, 11, or 12 nucleotides) downstream of the 5′-NTTN-3′ sequence and ends within about 20 to about 25 nucleotides (e.g., about 17, 18, 19, 20, 21, 22, 23, 24, 25, 26, 27, or 28 nucleotides) downstream of the 5′-NTTN-3′ sequence. In some embodiments, the deletion starts within about 5 to about 10 nucleotides (e.g., about 3, 4, 5, 6, 7, 8, 9, 10, 11, or 12 nucleotides) downstream of a 5′-ATTA-3′. 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′. 5′-CTTT-3′, 5′-CTTG-3′, or 5′-CTTC-3′ sequence and ends within about 20 to about 25 nucleotides (e.g., about 17, 18, 19, 20, 21, 22, 23, 24, 25, 26, 27, or 28 nucleotides) downstream of the 5′-ATTA-3′, 5′-ATTT-3′, 5′-ATTG-3′, 5′-ATTC-3′, 5′-TTTA-3′, 5′-TTTT-3′. 5′-TTTG-3′. 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 10 nucleotides (e.g., about 3, 4, 5, 6, 7, 8, 9, 10, 11, or 12 nucleotides) downstream of a T/C-rich sequence and ends within about 20 to about 25 nucleotides (e.g., about 17, 18, 19, 20, 21, 22, 23, 24, 25, 26, 27, or 28 nucleotides) downstream of the T/C-rich sequence.

In some embodiments, the deletion starts within about 5 to about 10 nucleotides (e.g., about 3, 4, 5, 6, 7, 8, 9, 10, 11, or 12 nucleotides) and ends within about 25 to about 30 nucleotides (e.g., about 22, 23, 24, 25, 26, 27, 28, 29, 30, 31, 32, or 33 nucleotides) of the 5′-NTTN-3′ sequence. In some embodiments, the deletion starts within about 5 to about 10 nucleotides (e.g., about 3, 4, 5, 6, 7, 8, 9, 10, 11, or 12 nucleotides) and ends within about 25 to about 30 nucleotides (e.g., about 22, 23, 24, 25, 26, 27, 28, 29, 30, 31, 32, or 33 nucleotides) of a T/C-rich sequence.

In some embodiments, the deletion starts within about 5 to about 10 nucleotides (e.g., about 3, 4, 5, 6, 7, 8, 9, 10, 11, or 12 nucleotides) downstream of the 5′-NTTN-3′ sequence and ends within about 25 to about 30 nucleotides (e.g., about 22, 23, 24, 25, 26, 27, 28, 29, 30, 31, 32, or 33 nucleotides) downstream of the 5′-NTTN-3′ sequence. In some embodiments, the deletion starts within about 5 to about 10 nucleotides (e.g., about 3, 4, 5, 6, 7, 8, 9, 10, 11, or 12 nucleotides) downstream of a 5′-ATTA-3′. 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence and ends within about 25 to about 30 nucleotides (e.g., about 22, 23, 24, 25, 26, 27, 28, 29, 30, 31, 32, or 33 nucleotides) downstream of the 5′-ATTA-3′. 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 10 nucleotides (e.g., about 3, 4, 5, 6, 7, 8, 9, 10, 11, or 12 nucleotides) downstream of a T/C-rich sequence and ends within about 25 to about 30 nucleotides (e.g., about 22, 23, 24, 25, 26, 27, 28, 29, 30, 31, 32, or 33 nucleotides) downstream of the T/C-rich sequence.

In some embodiments, the deletion starts within about 10 to about 15 nucleotides (e.g., about 8, 9, 10, 11, 12, 13, 14, 15, 16, or 17 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of the 5′-NTTN-3′ sequence. In some embodiments, the deletion starts within about 10 to about 15 nucleotides (e.g., about 8, 9, 10, 11, 12, 13, 14, 15, 16, or 17 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of a 5′-ATTA-3′, 5′-ATTT-3′, 5′-ATTG-3′, 5′-ATTC-3′. 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 10 to about 15 nucleotides (e.g., about 8, 9, 10, 11, 12, 13, 14, 15, 16, or 17 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of a T/C-rich sequence.

In some embodiments, the deletion starts within about 10 to about 15 nucleotides (e.g., about 8, 9, 10, 11, 12, 13, 14, 15, 16, or 17 nucleotides) downstream of the 5′-NTTN-3′ sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the 5′-NTTN-3′ sequence. In some embodiments, the deletion starts within about 10 to about 15 nucleotides (e.g., about 8, 9, 10, 11, 12, 13, 14, 15, 16, or 17 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 10 to about 15 nucleotides (e.g., about 8, 9, 10, 11, 12, 13, 14, 15, 16, or 17 nucleotides) downstream of a T/C-rich sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the T/C-rich sequence.

In some embodiments, the deletion starts within about 10 to about 15 nucleotides (e.g., about 8, 9, 10, 11, 12, 13, 14, 15, 16, or 17 nucleotides) and ends within about 20 to about 25 nucleotides (e.g., about 17, 18, 19, 20, 21, 22, 23, 24, 25, 26, 27, or 28 nucleotides) of the 5′-NTTN-3′ sequence. In some embodiments, the deletion starts within about 10 to about 15 nucleotides (e.g., about 8, 9, 10, 11, 12, 13, 14, 15, 16, or 17 nucleotides) and ends within about 20 to about 25 nucleotides (e.g., about 17, 18, 19, 20, 21, 22, 23, 24, 25, 26, 27, or 28 nucleotides) of a 5′-ATTA-3′. 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 10 to about 15 nucleotides (e.g., about 8, 9, 10, 11, 12, 13, 14, 15, 16, or 17 nucleotides) and ends within about 20 to about 25 nucleotides (e.g., about 17, 18, 19, 20, 21, 22, 23, 24, 25, 26, 27, or 28 nucleotides) of a T/C-rich sequence.

In some embodiments, the deletion starts within about 10 to about 15 nucleotides (e.g., about 8, 9, 10, 11, 12, 13, 14, 15, 16, or 17 nucleotides) downstream of the 5′-NTTN-3′ sequence and ends within about 20 to about 25 nucleotides (e.g., about 17, 18, 19, 20, 21, 22, 23, 24, 25, 26, 27, or 28 nucleotides) downstream of the 5′-NTTN-3′ sequence. In some embodiments, the deletion starts within about 10 to about 15 nucleotides (e.g., about 8, 9, 10, 11, 12, 13, 14, 15, 16, or 17 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′. 5′-GTTC-3′, 5′-CTTA-3′, 5′-CTTT-3′, 5′-CTTG-3′, or 5′-CTTC-3′ sequence and ends within about 20 to about 25 nucleotides (e.g., about 17, 18, 19, 20, 21, 22, 23, 24, 25, 26, 27, or 28 nucleotides) downstream of the 5′-ATTA-3′. 5′-ATTT-3′. 5′-ATTG-3′, 5′-ATTC-3′, 5′-TTTA-3′, 5′-TTTT-3′. 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 10 to about 15 nucleotides (e.g., about 8, 9, 10, 11, 12, 13, 14, 15, 16, or 17 nucleotides) downstream of a T/C-rich sequence and ends within about 20 to about 25 nucleotides (e.g., about 17, 18, 19, 20, 21, 22, 23, 24, 25, 26, 27, or 28 nucleotides) downstream of the T/C-rich sequence.

In some embodiments, the deletion starts within about 10 to about 15 nucleotides (e.g., about 8, 9, 10, 11, 12, 13, 14, 15, 16, or 17 nucleotides) and ends within about 25 to about 30 nucleotides (e.g., about 22, 23, 24, 25, 26, 27, 28, 29, 30, 31, 32, or 33 nucleotides) of the 5′-NTTN-3′ sequence. In some embodiments, the deletion starts within about 10 to about 15 nucleotides (e.g., about 8, 9, 10, 11, 12, 13, 14, 15, 16, or 17 nucleotides) and ends within about 25 to about 30 nucleotides (e.g., about 22, 23, 24, 25, 26, 27, 28, 29, 30, 31, 32, or 33 nucleotides) of a 5′-ATTA-3′. 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 10 to about 15 nucleotides (e.g., about 8, 9, 10, 11, 12, 13, 14, 15, 16, or 17 nucleotides) and ends within about 25 to about 30 nucleotides (e.g., about 22, 23, 24, 25, 26, 27, 28, 29, 30, 31, 32, or 33 nucleotides) of a T/C-rich sequence.

In some embodiments, the deletion starts within about 10 to about 15 nucleotides (e.g., about 8, 9, 10, 11, 12, 13, 14, 15, 16, or 17 nucleotides) downstream of the 5′-NTTN-3′ sequence and ends within about 25 to about 30 nucleotides (e.g., about 22, 23, 24, 25, 26, 27, 28, 29, 30, 31, 32, or 33 nucleotides) downstream of the 5′-NTTN-3′ sequence. In some embodiments, the deletion starts within about 10 to about 15 nucleotides (e.g., about 8, 9, 10, 11, 12, 13, 14, 15, 16, or 17 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence and ends within about 25 to about 30 nucleotides (e.g., about 22, 23, 24, 25, 26, 27, 28, 29, 30, 31, 32, or 33 nucleotides) downstream of the 5′-ATTA-3′, 5′-ATTT-3′, 5′-ATTG-3′, 5′-ATTC-3′. 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 10 to about 15 nucleotides (e.g., about 8, 9, 10, 11, 12, 13, 14, 15, 16, or 17 nucleotides) downstream of a T/C-rich sequence and ends within about 25 to about 30 nucleotides (e.g., about 22, 23, 24, 25, 26, 27, 28, 29, 30, 31, 32, or 33 nucleotides) downstream of the T/C-rich sequence.

In some embodiments, the deletion is up to about 40 nucleotides in length (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 nucleotides). In some embodiments, the deletion is between about 4 nucleotides and about 40 nucleotides in length (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 nucleotides). In some embodiments, the deletion is between about 4 nucleotides and about 25 nucleotides in length (e.g., about 3, 4, 5, 6, 7, 8, 9, 10, 11, 12, 13, 14, 15, 16, 17, 18, 19, 20, 21, 22, 23, 24, 25, 26, 27, or 28 nucleotides). In some embodiments, the deletion is between about 10 nucleotides and about 25 nucleotides in length (e.g., about 7, 8, 9, 10, 11, 12, 13, 14, 15, 16, 17, 18, 19, 20, 21, 22, 23, 24, 25, 26, 27, or 28 nucleotides). In some embodiments, the deletion is between about 10 nucleotides and about 15 nucleotides in length (e.g., about 7, 8, 9, 10, 11, 12, 13, 14, 15, 16, or 17 nucleotides).

In some embodiments, the methods described herein are used to engineer a cell comprising a deletion as described herein in an HAO1 gene. In some embodiments, the methods are carried out using a complex comprising a Cas12i enzyme as described herein and an RNA guide comprising a direct repeat and a spacer as described herein. In some embodiments, the sequence of the RNA guide has at least 90% identity (e.g., at least 90%, 91%, 92%, 93%, 94%. 95%, 96%, 97%, 98%, or 99% identity) to a sequence of any one of SEQ ID NOs: 967-1023. In some embodiments, an RNA guide has a sequence of any one of SEQ ID NOs: 967-1023.

In some embodiments, the RNA guide targeting HAO1 is encoded in a plasmid. In some embodiments, the RNA guide targeting HAO1 is synthetic or purified RNA. In some embodiments, the Cas12i polypeptide is encoded in a plasmid. In some embodiments, the Cas12i polypeptide is encoded by an RNA that is synthetic or purified.

### C. Delivery

Components of any of the gene editing systems disclosed herein may be formulated, for example, including a carrier, such as a carrier and/or a polymeric carrier, e.g., a liposome, and delivered by known methods to a cell (e.g., a prokaryotic, eukaryotic, plant, mammalian, etc.). Such methods include, but not limited to, transfection (e.g., lipid-mediated, cationic polymers, calcium phosphate, dendrimers); electroporation or other methods of membrane disruption (e.g., nucleofection), viral delivery (e.g., lentivirus, retrovirus, adenovirus, adeno-associated virus (AAV)), microinjection, microprojectile bombardment (“gene gun”), fugene, direct sonic loading, cell squeezing, optical transfection, protoplast fusion, impalefection, magnetofection, exosome-mediated transfer, lipid nanoparticle-mediated transfer, and any combination thereof.

In some embodiments, the method comprises delivering one or more nucleic acids (e.g., nucleic acids encoding the Cas12i polypeptide, RNA guide, donor DNA, etc.), one or more transcripts thereof, and/or a pre-formed RNA guide/Cas12i polypeptide complex to a cell, where a ternary complex is formed. In some embodiments, an RNA guide and an RNA encoding a Cas12i polypeptide are delivered together in a single composition. In some embodiments, an RNA guide and an RNA encoding a Cas12i polypeptide are delivered in separate compositions. In some embodiments, an RNA guide and an RNA encoding a Cas12i polypeptide delivered in separate compositions are delivered using the same delivery technology. In some embodiments, an RNA guide and an RNA encoding a Cas12i polypeptide delivered in separate compositions are delivered using different delivery technologies. Exemplary intracellular delivery methods, include, but are not limited to: viruses, such as AAV, or virus-like agents; chemical-based transfection methods, such as those using calcium phosphate, dendrimers, liposomes, lipid nanoparticles, or cationic polymers (e.g., DEAE-dextran or polyethylenimine); non-chemical methods, such as microinjection, electroporation, cell squeezing, sonoporation, optical transfection, impalefection, protoplast fusion, bacterial conjugation, delivery of plasmids or transposons; particle-based methods, such as using a gene gun, magnectofection or magnet assisted transfection, particle bombardment; and hybrid methods, such as nucleofection. In some embodiments, a lipid nanoparticle comprises an mRNA encoding a Cas12i polypeptide, an RNA guide, or an mRNA encoding a Cas12i polypeptide and an RNA guide. In some embodiments, the mRNA encoding the Cas12i polypeptide is a transcript of the nucleotide sequence set forth in SEQ ID NO: 921 or SEQ ID NO: 955 or a variant thereof. In some embodiments, the present application further provides cells produced by such methods, and organisms (such as animals, plants, or fungi) comprising or produced from such cells.

### D. Genetically Modified Cells

Any of the gene editing systems disclosed herein can be delivered to a variety of cells. In some embodiments, the cell is an isolated cell. In some embodiments, the cell is in cell culture or a co-culture of two or more cell types. In some embodiments, the cell is ex vivo. In some embodiments, the cell is obtained from a living organism and maintained in a cell culture. In some embodiments, the cell is a single-cellular organism.

In some embodiments, the cell is a prokaryotic cell. In some embodiments, the cell is a bacterial cell or derived from a bacterial cell. In some embodiments, the cell is an archaeal cell or derived from an archaeal cell.

In some embodiments, the cell is a eukaryotic cell. In some embodiments, the cell is a plant cell or derived from a plant cell. In some embodiments, the cell is a fungal cell or derived from a fungal cell. In some embodiments, the cell is an animal cell or derived from an animal cell. In some embodiments, the cell is an invertebrate cell or derived from an invertebrate cell. In some embodiments, the cell is a vertebrate cell or derived from a vertebrate cell. In some embodiments, the cell is a mammalian cell or derived from a mammalian cell. In some embodiments, the cell is a human cell. In some embodiments, the cell is a zebra fish cell. In some embodiments, the cell is a rodent cell. In some embodiments, the cell is synthetically made, sometimes termed an artificial cell.

In some embodiments, the cell is derived from a cell line. A wide variety of cell lines for tissue culture are known in the art. Examples of cell lines include, but are not limited to, 293T. MF7, K562, HeLa, CHO, and transgenic varieties thereof. Cell lines are available from a variety of sources known to those with skill in the art (see, e.g., the American Type Culture Collection (ATCC) (Manassas, Va.)). In some embodiments, the cell is an immortal or immortalized cell.

In some embodiments, the cell is a primary cell. In some embodiments, the cell is a stem cell such as a totipotent stem cell (e.g., omnipotent), a pluripotent stem cell, a multipotent stem cell, an oligopotent stem cell, or an unipotent stem cell. In some embodiments, the cell is an induced pluripotent stem cell (iPSC) or derived from an iPSC. In some embodiments, the cell is a differentiated cell. For example, in some embodiments, the differentiated cell is a liver cell (e.g., a hepatocyte), a biliary cell (e.g., a cholangiocyte), a stellate cell, a Kupffer cell, a liver sinusoidal endothelial cell, a muscle cell (e.g., a myocyte), a fat cell (e.g., an adipocyte), a bone cell (e.g., an osteoblast, osteocyte, osteoclast), a blood cell (e.g., a monocyte, a lymphocyte, a neutrophil, an cosinophil, a basophil, a macrophage, a erythrocyte, or a platelet), a nerve cell (e.g., a neuron), an epithelial cell, an immune cell (e.g., a lymphocyte, a neutrophil, a monocyte, or a macrophage), a fibroblast, or a sex cell. In some embodiments, the cell is a terminally differentiated cell. For example, in some embodiments, the terminally differentiated cell is a neuronal cell, an adipocyte, a cardiomyocyte, a skeletal muscle cell, an epidermal cell, or a gut cell. In some embodiments, the cell is an immune cell. In some embodiments, the immune cell is a T cell. In some embodiments, the immune cell is a B cell. In some embodiments, the immune cell is a Natural Killer (NK) cell. In some embodiments, the immune cell is a Tumor Infiltrating Lymphocyte (TIL). In some embodiments, the cell is a mammalian cell, e.g., a human cell or a murine cell. In some embodiments, the murine cell is derived from a wild-type mouse, an immunosuppressed mouse, or a disease-specific mouse model. In some embodiments, the cell is a cell within a living tissue, organ, or organism.

Any of the genetically modified cells produced using any of the gene editing system disclosed herein is also within the scope of the present disclosure. Such modified cells may comprise a disrupted HAO1 gene.

Compositions, vectors, nucleic acids, RNA guides and cells disclosed herein may be used in therapy. Compositions, vectors, nucleic acids, RNA guides and cells disclosed herein may be used in methods of treating a disease or condition in a subject. In some embodiments, the disease or condition is Any suitable delivery or administration method known in the art may be used to deliver compositions, vectors, nucleic acids, RNA guides and cells disclosed herein. Such methods may involve contacting a target sequence with a composition, vector, nucleic acid, or RNA guide disclosed herein. Such methods may involve a method of editing an HAO1 sequence as disclosed herein. In some embodiments, a cell engineered using an RNA guide disclosed herein is used for ex vivo gene therapy.

### IV. Therapeutic Applications

Any of the gene editing systems or modified cells generated using such a gene editing system as disclosed herein may be used for treating a disease that is associated with the HAO1 gene, for example, primary hyperoxaluria (PH). In some embodiments, the PH is PH1, PH2, or PH3. In specific examples, the target disease is PH1.

The gene editing system, pharmaceutical composition or kit comprising such, and any of the RNA guides disclosed herein may be used for treating primary hyperoxaluria (PH) in a subject. PH is a rare genetic disorder effecting subjects of all ages from infants to elderly. PH includes three subtypes involving genetic defects that alter the expression of three distinct proteins. PH1 involves alanine-glyoxylate aminotransferase, or AGT/AGT1. PH2 involves glyoxylate/hydroxypyruvate reductase, or GR/HPR, and PH3 involves 4-hydroxy-2-oxoglutarate aldolase, or HOGA.

In PH1, excess oxalate can also combine with calcium to form calcium oxalate in the kidney and other organs. Deposits of calcium oxalate can produce widespread deposition of calcium oxalate (nephrocalcinosis) or formation of kidney and bladder stones (urolithiasis) and lead to kidney damage. Common kidney complications in PH1 include blood in the urine (hematuria), urinary tract infections, kidney damage, and end-stage renal disease (ESRD). Over time, kidneys in patients with PH1 may begin to fail, and levels of oxalate may rise in the blood. Deposition of oxalate in tissues throughout the body, e.g., systemic oxalosis, may occur due to high blood levels of oxalate and can lead to complications in bone, skin, and eye. Patients with PH1 normally have kidney failure at an early age, with renal dialysis or dual kidney/liver organ transplant as the only treatment options.

In some embodiments, provided herein is a method for treating a target disease as disclosed herein (e.g., PH such as PH1) comprising administering to a subject (e.g., a human patient) in need of the treatment any of the gene editing systems disclosed herein. The gene editing system may be delivered to a specific tissue or specific type of cells where the gene edit is needed. The gene editing system may comprise LNPs encompassing one or more of the components, one or more vectors (e.g., viral vectors) encoding one or more of the components, or a combination thereof. Components of the gene editing system may be formulated to form a pharmaceutical composition, which may further comprise one or more pharmaceutically acceptable carriers.

In some embodiments, modified cells produced using any of the gene editing systems disclosed herein may be administered to a subject (e.g., a human patient) in need of the treatment. The modified cells may comprise a substitution, insertion, and/or deletion described herein. In some examples, the modified cells may include a cell line modified by a CRISPR nuclease, reverse transcriptase polypeptide, and editing template RNA (e.g., RNA guide and RT donor RNA). In some instances, the modified cells may be a heterogenous population comprising cells with different types of gene edits. Alternatively, the modified cells may comprise a substantially homogenous cell population (e.g., at least 80% of the cells in the whole population) comprising one particular gene edit in the HAO1 gene. In some examples, the cells can be suspended in a suitable media.

In some embodiments, provided herein is a composition comprising the gene editing system or components thereof. Such a composition can be a pharmaceutical composition. A pharmaceutical composition that is useful may be prepared, packaged, or sold in a formulation suitable for oral, rectal, vaginal, parenteral, topical, pulmonary, intranasal, intra-lesional, buccal, ophthalmic, intravenous, intra-organ or another route of administration. A pharmaceutical composition of the disclosure may be prepared, packaged, or sold in bulk, as a single unit dose, or as a plurality of single unit doses. As used herein, a “unit dose” is discrete amount of the pharmaceutical composition (e.g., the gene editing system or components thereof), which would be administered to a subject or a convenient fraction of such a dosage such as, for example, one-half or one-third of such a dosage.

In some embodiments, a pharmaceutical composition comprising the gene editing system or components thereof as described herein may be administered to a subject in need thereof, e.g., one who suffers from a liver disease associated with the HAO1 gene. In some instances, the gene editing system or components thereof may be delivered to specific cells or tissue (e.g., to liver cells), where the gene editing system could function to genetically modify the HAO1 gene in such cells.

A formulation of a pharmaceutical composition suitable for parenteral administration may comprise the active agent (e.g., the gene editing system or components thereof or the modified cells) combined with a pharmaceutically acceptable carrier, such as sterile water or sterile isotonic saline. Such a formulation may be prepared, packaged, or sold in a form suitable for bolus administration or for continuous administration. Some injectable formulations may be prepared, packaged, or sold in unit dosage form, such as in ampules or in multi-dose containers containing a preservative. Some formulations for parenteral administration include, but are not limited to, suspensions, solutions, emulsions in oily or aqueous vehicles, pastes, and implantable sustained-release or biodegradable formulations. Some formulations may further comprise one or more additional ingredients including, but not limited to, suspending, stabilizing, or dispersing agents.

The pharmaceutical composition may be in the form of a sterile injectable aqueous or oily suspension or solution. This suspension or solution may be formulated according to the known art, and may comprise, in addition to the cells, additional ingredients such as the dispersing agents, wetting agents, or suspending agents described herein. Such sterile injectable formulation may be prepared using a non-toxic parenterally-acceptable diluent or solvent, such as water or saline. Other acceptable diluents and solvents include, but are not limited to, Ringer's solution, isotonic sodium chloride solution, and fixed oils such as synthetic mono- or di-glycerides. Other parentally-administrable formulations which that are useful include those which may comprise the cells in a packaged form, in a liposomal preparation, or as a component of a biodegradable polymer system. Some compositions for sustained release or implantation may comprise pharmaceutically acceptable polymeric or hydrophobic materials such as an emulsion, an ion exchange resin, a sparingly soluble polymer, or a sparingly soluble salt.

### V. Kits and Uses Thereof

The present disclosure also provides kits that can be used, for example, to carry out a method described herein for genetical modification of the HAO1 gene. In some embodiments, the kits include an RNA guide and a Cas12i polypeptide. In some embodiments, the kits include a polynucleotide that encodes such a Cas12i polypeptide, and optionally the polynucleotide is comprised within a vector, e.g., as described herein. The Cas12i polypeptide and the RNA guide (e.g., as a ribonucleoprotein) can be packaged within the same or other vessel within a kit or system or can be packaged in separate vials or other vessels, the contents of which can be mixed prior to use. The kits can additionally include, optionally, a buffer and/or instructions for use of the RNA guide and Cas12i polypeptide.

In some embodiments, the kit may be useful for research purposes. For example, in some embodiments, the kit may be useful to study gene function.

All references and publications cited herein are hereby incorporated by reference.

### Additional Embodiments

Provided below are additional embodiments, which are also within the scope of the present disclosure.

Embodiment 1: A composition comprising an RNA guide, wherein the RNA guide comprises (i) a spacer sequence that is substantially complementary or completely complementary to a region on a non-PAM strand (the complementary sequence of a target sequence) within an HAO1 gene and (ii) a direct repeat sequence; wherein the target sequence is adjacent to a protospacer adjacent motif (PAM) comprising the sequence 5′-NTTN-3′.

In Embodiment 1, the target sequence may be within exon 1, exon 2, exon 3, exon 4, exon 5, exon 6, or exon 7 of the HAO1 gene. In some examples, the HAO1 gene comprises the sequence of SEQ ID NO: 928, the reverse complement of SEQ ID NO: 928, a variant of SEQ ID NO: 928, or the reverse complement of a variant of SEQ ID NO: 928.

In Embodiment 1, the spacer sequence may comprise: (a) nucleotide 1 through nucleotide 16 of a sequence that is at least 90% identical to a sequence of any one of SEQ ID NOs: 466-920; (b) nucleotide 1 through nucleotide 17 of a sequence that is at least 90% identical to a sequence of any one of SEQ ID NOs: 466-920; (c) nucleotide 1 through nucleotide 18 of a sequence that is at least 90% identical to a sequence of any one of SEQ ID NOs: 466-920; (d) nucleotide 1 through nucleotide 19 of a sequence that is at least 90% identical to a sequence of any one of SEQ ID NOs: 466-920; (e) nucleotide 1 through nucleotide 20 of a sequence that is at least 90% identical to a sequence of any one of SEQ ID NOs: 466-920; (f) nucleotide 1 through nucleotide 21 of a sequence that is at least 90% identical to a sequence of any one of SEQ ID NOs: 466-920; (g) nucleotide 1 through nucleotide 22 of a sequence that is at least 90% identical to a sequence of any one of SEQ ID NOs: 466-920; (h) nucleotide 1 through nucleotide 23 of a sequence that is at least 90% identical to a sequence of any one of SEQ ID NOs: 466-920; (i) nucleotide 1 through nucleotide 24 of a sequence that is at least 90% identical to a sequence of any one of SEQ ID NOs: 466-920; (j) nucleotide 1 through nucleotide 25 of a sequence that is at least 90% identical to a sequence of any one of SEQ ID NOs: 466-920; (k) nucleotide 1 through nucleotide 26 of a sequence that is at least 90% identical to a sequence of any one of SEQ ID NOs: 466-920; (l) nucleotide 1 through nucleotide 27 of a sequence that is at least 90% identical to a sequence of any one of SEQ ID NOs: 466-920; (m) nucleotide 1 through nucleotide 28 of a sequence that is at least 90% identical to a sequence of any one of SEQ ID NOs: 466-920; (n) nucleotide 1 through nucleotide 29 of a sequence that is at least 90% identical to a sequence of any one of SEQ ID NOs: 466-920; or (o) nucleotide 1 through nucleotide 30 of a sequence that is at least 90% identical to a sequence of any one of SEQ ID NOs: 466-920.

In any of the composition of Embodiment 1, the spacer sequence may comprise: (a) nucleotide 1 through nucleotide 16 of any one of SEQ ID NOs: 466-920; (b) nucleotide 1 through nucleotide 17 of any one of SEQ ID NOs: 466-920; (c) nucleotide 1 through nucleotide 18 of any one of SEQ ID NOs: 466-920; (d) nucleotide 1 through nucleotide 19 of any one of SEQ ID NOs: 466-920; (c) nucleotide 1 through nucleotide 20 of any one of SEQ ID NOs: 466-920; (f) nucleotide 1 through nucleotide 21 of any one of SEQ ID NOs: 466-920; (g) nucleotide 1 through nucleotide 22 of any one of SEQ ID NOs: 466-920; (h) nucleotide 1 through nucleotide 23 of any one of SEQ ID NOs: 466-920; (i) nucleotide 1 through nucleotide 24 of any one of SEQ ID NOs: 466-920; (j) nucleotide 1 through nucleotide 25 of any one of SEQ ID NOS: 466-920; (k) nucleotide 1 through nucleotide 26 of any one of SEQ ID NOs: 466-920; (l) nucleotide 1 through nucleotide 27 of any one of SEQ ID NOs: 466-920; (m) nucleotide 1 through nucleotide 28 of any one of SEQ ID NOs: 466-920; (n) nucleotide 1 through nucleotide 29 of any one of SEQ ID NOs: 466-920; or (o) nucleotide 1 through nucleotide 30 of any one of SEQ ID NOs: 466-920.

In any of the composition of Embodiment 1, the direct repeat sequence may comprises: (a) nucleotide 1 through nucleotide 36 of a sequence that is at least 90% identical to a sequence of any one of SEQ ID NOs: 1-8; (b) nucleotide 2 through nucleotide 36 of a sequence that is at least 90% identical to a sequence of any one of SEQ ID NOs: 1-8; (c) nucleotide 3 through nucleotide 36 of a sequence that is at least 90% identical to a sequence of any one of SEQ ID NOs: 1-8; (d) nucleotide 4 through nucleotide 36 of a sequence that is at least 90% identical to a sequence of any one of SEQ ID NOs: 1-8; (e) nucleotide 5 through nucleotide 36 of a sequence that is at least 90% identical to a sequence of any one of SEQ ID NOs: 1-8; (f) nucleotide 6 through nucleotide 36 of a sequence that is at least 90% identical to a sequence of any one of SEQ ID NOs: 1-8; (g) nucleotide 7 through nucleotide 36 of a sequence that is at least 90% identical to a sequence of any one of SEQ ID NOs: 1-8; (h) nucleotide 8 through nucleotide 36 of a sequence that is at least 90% identical to a sequence of any one of SEQ ID NOs: 1-8; (i) nucleotide 9 through nucleotide 36 of a sequence that is at least 90% identical to a sequence of any one of SEQ ID NOs: 1-8; (j) nucleotide 10 through nucleotide 36 of a sequence that is at least 90% identical to a sequence of any one of SEQ ID NOs: 1-8; (k) nucleotide 11 through nucleotide 36 of a sequence that is at least 90% identical to a sequence of any one of SEQ ID NOs: 1-8; (l) nucleotide 12 through nucleotide 36 of a sequence that is at least 90% identical to a sequence of any one of SEQ ID NOs: 1-8; (m) nucleotide 13 through nucleotide 36 of a sequence that is at least 90% identical to a sequence of any one of SEQ ID NOs: 1-8; (n) nucleotide 14 through nucleotide 36 of a sequence that is at least 90% identical to a sequence of any one of SEQ ID NOs: 1-8; (o) nucleotide 1 through nucleotide 34 of a sequence that is at least 90% identical to a sequence of SEQ ID NO: 9; (p) nucleotide 2 through nucleotide 34 of a sequence that is at least 90% identical to a sequence of SEQ ID NO: 9; (q) nucleotide 3 through nucleotide 34 of a sequence that is at least 90% identical to a sequence of SEQ ID NO: 9; (r) nucleotide 4 through nucleotide 34 of a sequence that is at least 90% identical to a sequence of SEQ ID NO: 9; (s) nucleotide 5 through nucleotide 34 of a sequence that is at least 90% identical to a sequence of SEQ ID NO: 9; (t) nucleotide 6 through nucleotide 34 of a sequence that is at least 90% identical to a sequence of SEQ ID NO: 9; (u) nucleotide 7 through nucleotide 34 of a sequence that is at least 90% identical to a sequence of SEQ ID NO: 9; (v) nucleotide 8 through nucleotide 34 of a sequence that is at least 90% identical to a sequence of SEQ ID NO: 9; (w) nucleotide 9 through nucleotide 34 of a sequence that is at least 90% identical to a sequence of SEQ ID NO: 9; (x) nucleotide 10 through nucleotide 34 of a sequence that is at least 90% identical to a sequence of SEQ ID NO: 9; (y) nucleotide 11 through nucleotide 34 of a sequence that is at least 90% identical to a sequence of SEQ ID NO: 9; (2) nucleotide 12 through nucleotide 34 of a sequence that is at least 90% identical to a sequence of SEQ ID NO: 9; or (aa) a sequence that is at least 90% identical to a sequence of SEQ ID NO: 10 or a portion thereof.

In some examples, the direct repeat sequence comprises: (a) nucleotide 1 through nucleotide 36 of any one of SEQ ID NOs: 1-8; (b) nucleotide 2 through nucleotide 36 of any one of SEQ ID NOs: 1-8; (c) nucleotide 3 through nucleotide 36 of any one of SEQ ID NOs: 1-8; (d) nucleotide 4 through nucleotide 36 of any one of SEQ ID NOs: 1-8; (e) nucleotide 5 through nucleotide 36 of any one of SEQ ID NOs: 1-8; (f) nucleotide 6 through nucleotide 36 of any one of SEQ ID NOs: 1-8; (g) nucleotide 7 through nucleotide 36 of any one of SEQ ID NOs: 1-8; (h) nucleotide 8 through nucleotide 36 of any one of SEQ ID NOs: 1-8; (i) nucleotide 9 through nucleotide 36 of any one of SEQ ID NOs: 1-8; (j) nucleotide 10 through nucleotide 36 of any one of SEQ ID NOs: 1-8; (k) nucleotide 11 through nucleotide 36 of any one of SEQ ID NOs: 1-8; (l) nucleotide 12 through nucleotide 36 of any one of SEQ ID NOs: 1-8; (m) nucleotide 13 through nucleotide 36 of any one of SEQ ID NOs: 1-8; (n) nucleotide 14 through nucleotide 36 of any one of SEQ ID NOs: 1-8; (o) nucleotide 1 through nucleotide 34 of SEQ ID NO: 9; (p) nucleotide 2 through nucleotide 34 of SEQ ID NO: 9; (q) nucleotide 3 through nucleotide 34 of SEQ ID NO: 9; (r) nucleotide 4 through nucleotide 34 of SEQ ID NO: 9; (s) nucleotide 5 through nucleotide 34 of SEQ ID NO: 9; (t) nucleotide 6 through nucleotide 34 of SEQ ID NO: 9; (u) nucleotide 7 through nucleotide 34 of SEQ ID NO: 9; (v) nucleotide 8 through nucleotide 34 of SEQ ID NO: 9; (w) nucleotide 9 through nucleotide 34 of SEQ ID NO: 9; (x) nucleotide 10 through nucleotide 34 of SEQ ID NO: 9; (y) nucleotide 11 through nucleotide 34 of SEQ ID NO: 9; (z) nucleotide 12 through nucleotide 34 of SEQ ID NO: 9; (or aa) SEQ ID NO: 10 or a portion thereof).

In some examples, the direct repeat sequence comprises: (a) nucleotide 1 through nucleotide 36 of a sequence that is at least 90% identical to a sequence of any one of SEQ ID NOs: 936-953; (b) nucleotide 2 through nucleotide 36 of a sequence that is at least 90% identical to a sequence of any one of SEQ ID NOs: 936-953; (c) nucleotide 3 through nucleotide 36 of a sequence that is at least 90% identical to a sequence of any one of SEQ ID NOs: 936-953; (d) nucleotide 4 through nucleotide 36 of a sequence that is at least 90% identical to a sequence of any one of SEQ ID NOs: 936-953; (e) nucleotide 5 through nucleotide 36 of a sequence that is at least 90% identical to a sequence of any one of SEQ ID NOs: 936-953; (f) nucleotide 6 through nucleotide 36 of a sequence that is at least 90% identical to a sequence of any one of SEQ ID NOs: 936-953; (g) nucleotide 7 through nucleotide 36 of a sequence that is at least 90% identical to a sequence of any one of SEQ ID NOs: 936-953; (h) nucleotide 8 through nucleotide 36 of a sequence that is at least 90% identical to a sequence of any one of SEQ ID NOs: 936-953; (i) nucleotide 9 through nucleotide 36 of a sequence that is at least 90% identical to a sequence of any one of SEQ ID NOs: 936-953; (j) nucleotide 10 through nucleotide 36 of a sequence that is at least 90% identical to a sequence of any one of SEQ ID NOs: 936-953; (k) nucleotide 11 through nucleotide 36 of a sequence that is at least 90% identical to a sequence of any one of SEQ ID NOs: 936-953; (l) nucleotide 12 through nucleotide 36 of a sequence that is at least 90% identical to a sequence of any one of SEQ ID NOs: 936-953; (m) nucleotide 13 through nucleotide 36 of a sequence that is at least 90% identical to a sequence of any one of SEQ ID NOs: 936-953; (n) nucleotide 14 through nucleotide 36 of a sequence that is at least 90% identical to a sequence of any one of SEQ ID NOs: 936-953; or (o) a sequence that is at least 90% identical to a sequence of SEQ ID NO: 954 or a portion thereof).

In some examples, the direct repeat sequence comprises: (a) nucleotide 1 through nucleotide 36 of any one of SEQ ID NOs: 936-953; (b) nucleotide 2 through nucleotide 36 of any one of SEQ ID NOs: 936-953; (c) nucleotide 3 through nucleotide 36 of any one of SEQ ID NOs: 936-953; (d) nucleotide 4 through nucleotide 36 of any one of SEQ ID NOs: 936-953; (e) nucleotide 5 through nucleotide 36 of any one of SEQ ID NOs: 936-953; (f) nucleotide 6 through nucleotide 36 of any one of SEQ ID NOs: 936-953; (g) nucleotide 7 through nucleotide 36 of any one of SEQ ID NOs: 936-953; (h) nucleotide 8 through nucleotide 36 of any one of SEQ ID NOs: 936-953; (i) nucleotide 9 through nucleotide 36 of any one of SEQ ID NOs: 936-953; (j) nucleotide 10 through nucleotide 36 of any one of SEQ ID NOs: 936-953; (k) nucleotide 11 through nucleotide 36 of any one of SEQ ID NOs: 936-953; (l) nucleotide 12 through nucleotide 36 of any one of SEQ ID NOs: 936-953; (m) nucleotide 13 through nucleotide 36 of any one of SEQ ID NOs: 936-953; (n) nucleotide 14 through nucleotide 36 of any one of SEQ ID NOs: 936-953; (or o) SEQ ID NO: 954 or a portion thereof.

In some examples, the direct repeat sequence comprises: (a) nucleotide 1 through nucleotide 36 of a sequence that is at least 90% identical to SEQ ID NO: 959; (b) nucleotide 2 through nucleotide 36 of a sequence that is at least 90% identical to SEQ ID NO: 959; (c) nucleotide 3 through nucleotide 36 of a sequence that is at least 90% identical to SEQ ID NO: 959; (d) nucleotide 4 through nucleotide 36 of a sequence that is at least 90% identical to SEQ ID NO: 959; (c) nucleotide 5 through nucleotide 36 of a sequence that is at least 90% identical to SEQ ID NO: 959; (f) nucleotide 6 through nucleotide 36 of a sequence that is at least 90% identical to SEQ ID NO: 959; (g) nucleotide 7 through nucleotide 36 of a sequence that is at least 90% identical to SEQ ID NO: 959; (h) nucleotide 8 through nucleotide 36 of a sequence that is at least 90% identical to SEQ ID NO: 959; (i) nucleotide 9 through nucleotide 36 of a sequence that is at least 90% identical to SEQ ID NO: 959; (j) nucleotide 10 through nucleotide 36 of a sequence that is at least 90% identical to SEQ ID NO: 959; (k) nucleotide 11 through nucleotide 36 of a sequence that is at least 90% identical to SEQ ID NO: 959; (l) nucleotide 12 through nucleotide 36 of a sequence that is at least 90% identical to SEQ ID NO: 959; (m) nucleotide 13 through nucleotide 36 of a sequence that is at least 90% identical to SEQ ID NO: 959; (n) nucleotide 14 through nucleotide 36 of a sequence that is at least 90% identical to SEQ ID NO: 959; or (o) a sequence that is at least 90% identical to a sequence of SEQ ID NO: 960 or SEQ ID NO: 961 or a portion thereof).

In some examples, the direct repeat sequence comprises: (a) nucleotide 1 through nucleotide 36 of SEQ ID NO: 959; (b) nucleotide 2 through nucleotide 36 of SEQ ID NO: 959; (c) nucleotide 3 through nucleotide 36 of SEQ ID NO: 959; (d) nucleotide 4 through nucleotide 36 of SEQ ID NO: 959; (e) nucleotide 5 through nucleotide 36 of SEQ ID NO: 959; (f) nucleotide 6 through nucleotide 36 of SEQ ID NO: 959; (g) nucleotide 7 through nucleotide 36 of SEQ ID NO: 959; (h) nucleotide 8 through nucleotide 36 of SEQ ID NO: 959; (i) nucleotide 9 through nucleotide 36 of SEQ ID NO: 959; (j) nucleotide 10 through nucleotide 36 of SEQ ID NO: 959; (k) nucleotide 11 through nucleotide 36 of SEQ ID NO: 959; (l) nucleotide 12 through nucleotide 36 of SEQ ID NO: 959; (m) nucleotide 13 through nucleotide 36 of SEQ ID NO: 959; (n) nucleotide 14 through nucleotide 36 of SEQ ID NO: 959; or (o) SEQ ID NO: 960 or SEQ ID NO: 961 or a portion thereof).

In some examples, the direct repeat sequence comprises: (a) nucleotide 1 through nucleotide 36 of a sequence that is at least 90% identical to a sequence of SEQ ID NO: 962 or SEQ ID NO: 963; (b) nucleotide 2 through nucleotide 36 of a sequence that is at least 90% identical to a sequence of SEQ ID NO: 962 or SEQ ID NO: 963; (c) nucleotide 3 through nucleotide 36 of a sequence that is at least 90% identical to a sequence of SEQ ID NO: 962 or SEQ ID NO: 963; (d) nucleotide 4 through nucleotide 36 of a sequence that is at least 90% identical to a sequence of SEQ ID NO: 962 or SEQ ID NO: 963; (e) nucleotide 5 through nucleotide 36 of a sequence that is at least 90% identical to a sequence of SEQ ID NO: 962 or SEQ ID NO: 963; (f) nucleotide 6 through nucleotide 36 of a sequence that is at least 90% identical to a sequence of SEQ ID NO: 962 or SEQ ID NO: 963; (g) nucleotide 7 through nucleotide 36 of a sequence that is at least 90% identical to a sequence of SEQ ID NO: 962 or SEQ ID NO: 963; (h) nucleotide 8 through nucleotide 36 of a sequence that is at least 90% identical to a sequence of SEQ ID NO: 962 or SEQ ID NO: 963; (i) nucleotide 9 through nucleotide 36 of a sequence that is at least 90% identical to a sequence of SEQ ID NO: 962 or SEQ ID NO: 963; (j) nucleotide 10 through nucleotide 36 of a sequence that is at least 90% identical to a sequence of SEQ ID NO: 962 or SEQ ID NO: 963; (k) nucleotide 11 through nucleotide 36 of a sequence that is at least 90% identical to a sequence of SEQ ID NO: 962 or SEQ ID NO: 963; (l) nucleotide 12 through nucleotide 36 of a sequence that is at least 90% identical to a sequence of SEQ ID NO: 962 or SEQ ID NO: 963; (m) nucleotide 13 through nucleotide 36 of a sequence that is at least 90% identical to a sequence of SEQ ID NO: 962 or SEQ ID NO: 963; (n) nucleotide 14 through nucleotide 36 of a sequence that is at least 90% identical to a sequence of SEQ ID NO: 962 or SEQ ID NO: 963; (o) nucleotide 15 through nucleotide 36 of a sequence that is at least 90% identical to a sequence of SEQ ID NO: 962 or SEQ ID NO: 963; or (p) a sequence that is at least 90% identical to a sequence of SEQ ID NO: 964 or a portion thereof).

In some examples, the direct repeat sequence comprises: (a) nucleotide 1 through nucleotide 36 of SEQ ID NO: 962 or SEQ ID NO: 963; (b) nucleotide 2 through nucleotide 36 of SEQ ID NO: 962 or SEQ ID NO: 963; (c) nucleotide 3 through nucleotide 36 of SEQ ID NO: 962 or SEQ ID NO: 963; (d) nucleotide 4 through nucleotide 36 of SEQ ID NO: 962 or SEQ ID NO: 963; (c) nucleotide 5 through nucleotide 36 of SEQ ID NO: 962 or SEQ ID NO: 963; (f) nucleotide 6 through nucleotide 36 of SEQ ID NO: 962 or SEQ ID NO: 963; (g) nucleotide 7 through nucleotide 36 of SEQ ID NO: 962 or SEQ ID NO: 963; (h) nucleotide 8 through nucleotide 36 of SEQ ID NO: 962 or SEQ ID NO: 963; (i) nucleotide 9 through nucleotide 36 of SEQ ID NO: 962 or SEQ ID NO: 963; (j) nucleotide 10 through nucleotide 36 of SEQ ID NO: 962 or SEQ ID NO: 963; (k) nucleotide 11 through nucleotide 36 of SEQ ID NO: 962 or SEQ ID NO: 963; (l) nucleotide 12 through nucleotide 36 of SEQ ID NO: 962 or SEQ ID NO: 963; (m) nucleotide 13 through nucleotide 36 of SEQ ID NO: 962 or SEQ ID NO: 963; (n) nucleotide 14 through nucleotide 36 of SEQ ID NO: 962 or SEQ ID NO: 963; (o) nucleotide 15 through nucleotide 36 of SEQ ID NO: 962 or SEQ ID NO: 963; or (p) SEQ ID NO: 964 or a portion thereof).

In some examples, the spacer sequence is substantially complementary or completely complementary to the complement of a sequence of any one of SEQ ID NOs: 11-465.

In any of the composition of Embodiment 1, the PAM may comprise the sequence 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′.

In some examples, the target sequence is immediately adjacent to the PAM sequence.

In some examples, the RNA guide has a sequence that is at least 90% identical to a sequence of any one of SEQ ID NOs: 967-1023.

In some examples, the RNA guide has the sequence of any one of SEQ ID NOs: 967-1023.

Embodiment 2: The composition of Embodiment 1 may further comprise a Cas12i polypeptide or a polyribonucleotide encoding a Cas12i polypeptide, which can be one of the following: (a) a Cas12i2 polypeptide comprising a sequence that is at least 90% identical to the sequence of SEQ ID NO: 922, SEQ ID NO: 923, SEQ ID NO: 924, SEQ ID NO: 925, SEQ ID NO: 926, or SEQ ID NO: 927; (b) a Cas12i4 polypeptide comprising a sequence that is at least 90% identical to the sequence of SEQ ID NO: 956, SEQ ID NO: 957, or SEQ ID NO: 958; (c) a Cas12i1 polypeptide comprising a sequence that is at least 90% identical to the sequence of SEQ ID NO: 965; or (d) a Cas12i3 polypeptide comprising a sequence that is at least 90% identical to the sequence of SEQ ID NO: 966.

In specific examples, the Cas12i polypeptide is: (a) a Cas12i2 polypeptide comprising a sequence of SEQ ID NO: 922, SEQ ID NO: 923, SEQ ID NO: 924, SEQ ID NO: 925, SEQ ID NO: 926, or SEQ ID NO: 927; (b) a Cas1214 polypeptide comprising a sequence of SEQ ID NO: 956, SEQ ID NO: 957, or SEQ ID NO: 958; (c) a Cas12i1 polypeptide comprising a sequence of SEQ ID NO: 965; or (d) a Cas12i3 polypeptide comprising a sequence of SEQ ID NO: 966.

In any of the compositions of Embodiment 2, the RNA guide and the Cas12i polypeptide may form a ribonucleoprotein complex. In some examples, the ribonucleoprotein complex binds a target nucleic acid. In some examples, the composition is present within a cell.

In any of the compositions of Embodiment 2, the RNA guide and the Cas12i polypeptide may be encoded in a vector, e.g., expression vector. In some examples, the RNA guide and the Cas12i polypeptide are encoded in a single vector. In other examples, the RNA guide is encoded in a first vector and the Cas12i polypeptide is encoded in a second vector.

Embodiment 3: A vector system comprising one or more vectors encoding an RNA guide disclosed herein and a Cas12i polypeptide. In some examples, the vector system comprises a first vector encoding an RNA guide disclosed herein and a second vector encoding a Cas12i polypeptide. The vectors may be expression vectors.

Embodiment 4: A composition comprising an RNA guide and a Cas12i polypeptide, wherein the RNA guide comprises (i) a spacer sequence that is substantially complementary or completely complementary to a region on a non-PAM strand (the complementary sequence of a target sequence) within an HAO1 gene; and (ii) a direct repeat sequence.

In some examples, the target sequence is within exon 1, exon 2, exon 3, exon 4, exon 5, exon 6, or exon 7 of the HAO1 gene, which may comprise the sequence of SEQ ID NO: 928, the reverse complement of SEQ ID NO: 928, a variant of the sequence of SEQ ID NO: 928, or the reverse complement of a variant of SEQ ID NO: 928.

In some examples, the spacer sequence comprises: (a) nucleotide 1 through nucleotide 16 of a sequence that is at least 90% identical to a sequence of any one of SEQ ID NOs: 466-920; (b) nucleotide 1 through nucleotide 17 of a sequence that is at least 90% identical to a sequence of any one of SEQ ID NOs: 466-920; (c) nucleotide 1 through nucleotide 18 of a sequence that is at least 90% identical to a sequence of any one of SEQ ID NOs: 466-920; (d) nucleotide 1 through nucleotide 19 of a sequence that is at least 90% identical to a sequence of any one of SEQ ID NOs: 466-920; (e) nucleotide 1 through nucleotide 20 of a sequence that is at least 90% identical to a sequence of any one of SEQ ID NOs: 466-920; (f) nucleotide 1 through nucleotide 21 of a sequence that is at least 90% identical to a sequence of any one of SEQ ID NOs: 466-920; (g) nucleotide 1 through nucleotide 22 of a sequence that is at least 90% identical to a sequence of any one of SEQ ID NOs: 466-920; (h) nucleotide 1 through nucleotide 23 of a sequence that is at least 90% identical to a sequence of any one of SEQ ID NOs: 466-920; (i) nucleotide 1 through nucleotide 24 of a sequence that is at least 90% identical to a sequence of any one of SEQ ID NOs: 466-920; (j) nucleotide 1 through nucleotide 25 of a sequence that is at least 90% identical to a sequence of any one of SEQ ID NOs: 466-920; (k) nucleotide 1 through nucleotide 26 of a sequence that is at least 90% identical to a sequence of any one of SEQ ID NOs: 466-920; (l) nucleotide 1 through nucleotide 27 of a sequence that is at least 90% identical to a sequence of any one of SEQ ID NOs: 466-920; (m) nucleotide 1 through nucleotide 28 of a sequence that is at least 90% identical to a sequence of any one of SEQ ID NOs: 466-920; (n) nucleotide 1 through nucleotide 29 of a sequence that is at least 90% identical to a sequence of any one of SEQ ID NOs: 466-920; or (o) nucleotide 1 through nucleotide 30 of a sequence that is at least 90% identical to a sequence of any one of SEQ ID NOs: 466-920.

In some examples, the spacer sequence comprises: (a) nucleotide 1 through nucleotide 16 of any one of SEQ ID NOs: 466-920; (b) nucleotide 1 through nucleotide 17 of any one of SEQ ID NOs: 466-920; (c) nucleotide 1 through nucleotide 18 of any one of SEQ ID NOs: 466-920; (d) nucleotide 1 through nucleotide 19 of any one of SEQ ID NOs: 466-920; (e) nucleotide 1 through nucleotide 20 of any one of SEQ ID NOs: 466-920; (f) nucleotide 1 through nucleotide 21 of any one of SEQ ID NOs: 466-920; (g) nucleotide 1 through nucleotide 22 of any one of SEQ ID NOs: 466-920; (h) nucleotide 1 through nucleotide 23 of any one of SEQ ID NOs: 466-920; (i) nucleotide 1 through nucleotide 24 of any one of SEQ ID NOs: 466-920; (j) nucleotide 1 through nucleotide 25 of any one of SEQ ID NOs: 466-920; (k) nucleotide 1 through nucleotide 26 of any one of SEQ ID NOs: 466-920; (l) nucleotide 1 through nucleotide 27 of any one of SEQ ID NOs: 466-920; (m) nucleotide 1 through nucleotide 28 of any one of SEQ ID NOs: 466-920; (n) nucleotide 1 through nucleotide 29 of any one of SEQ ID NOs: 466-920; or (o) nucleotide 1 through nucleotide 30 of any one of SEQ ID NOs: 466-920.

In some examples, the direct repeat sequence comprises: (a) nucleotide 1 through nucleotide 36 of a sequence that is at least 90% identical to a sequence of any one of SEQ ID NOs: 1-8; (b) nucleotide 2 through nucleotide 36 of a sequence that is at least 90% identical to a sequence of any one of SEQ ID NOs: 1-8; (c) nucleotide 3 through nucleotide 36 of a sequence that is at least 90% identical to a sequence of any one of SEQ ID NOs: 1-8; (d) nucleotide 4 through nucleotide 36 of a sequence that is at least 90% identical to a sequence of any one of SEQ ID NOs: 1-8; (e) nucleotide 5 through nucleotide 36 of a sequence that is at least 90% identical to a sequence of any one of SEQ ID NOs: 1-8; (f) nucleotide 6 through nucleotide 36 of a sequence that is at least 90% identical to a sequence of any one of SEQ ID NOs: 1-8; (g) nucleotide 7 through nucleotide 36 of a sequence that is at least 90% identical to a sequence of any one of SEQ ID NOs: 1-8; (h) nucleotide 8 through nucleotide 36 of a sequence that is at least 90% identical to a sequence of any one of SEQ ID NOs: 1-8; (i) nucleotide 9 through nucleotide 36 of a sequence that is at least 90% identical to a sequence of any one of SEQ ID NOs: 1-8; (j) nucleotide 10 through nucleotide 36 of a sequence that is at least 90% identical to a sequence of any one of SEQ ID NOs: 1-8; (k) nucleotide 11 through nucleotide 36 of a sequence that is at least 90% identical to a sequence of any one of SEQ ID NOs: 1-8; (l) nucleotide 12 through nucleotide 36 of a sequence that is at least 90% identical to a sequence of any one of SEQ ID NOs: 1-8; (m) nucleotide 13 through nucleotide 36 of a sequence that is at least 90% identical to a sequence of any one of SEQ ID NOs: 1-8; (n) nucleotide 14 through nucleotide 36 of a sequence that is at least 90% identical to a sequence of any one of SEQ ID NOs: 1-8; (o) nucleotide 1 through nucleotide 34 of a sequence that is at least 90% identical to a sequence of SEQ ID NO: 9; (p) nucleotide 2 through nucleotide 34 of a sequence that is at least 90% identical to a sequence of SEQ ID NO: 9; (q) nucleotide 3 through nucleotide 34 of a sequence that is at least 90% identical to a sequence of SEQ ID NO: 9; (r) nucleotide 4 through nucleotide 34 of a sequence that is at least 90% identical to a sequence of SEQ ID NO: 9; (s) nucleotide 5 through nucleotide 34 of a sequence that is at least 90% identical to a sequence of SEQ ID NO: 9; (t) nucleotide 6 through nucleotide 34 of a sequence that is at least 90% identical to a sequence of SEQ ID NO: 9; (u) nucleotide 7 through nucleotide 34 of a sequence that is at least 90% identical to a sequence of SEQ ID NO: 9; (v) nucleotide 8 through nucleotide 34 of a sequence that is at least 90% identical to a sequence of SEQ ID NO: 9; (w) nucleotide 9 through nucleotide 34 of a sequence that is at least 90% identical to a sequence of SEQ ID NO: 9; (x) nucleotide 10 through nucleotide 34 of a sequence that is at least 90% identical to a sequence of SEQ ID NO: 9; (y) nucleotide 11 through nucleotide 34 of a sequence that is at least 90% identical to a sequence of SEQ ID NO: 9; (z) nucleotide 12 through nucleotide 34 of a sequence that is at least 90% identical to a sequence of SEQ ID NO: 9; or (aa) a sequence that is at least 90% identical to a sequence of SEQ ID NO: 10 or a portion thereof).

In some examples, the direct repeat sequence comprises: (a) nucleotide 1 through nucleotide 36 of any one of SEQ ID NOs: 1-8; (b) nucleotide 2 through nucleotide 36 of any one of SEQ ID NOs: 1-8; (c) nucleotide 3 through nucleotide 36 of any one of SEQ ID NOs: 1-8; (d) nucleotide 4 through nucleotide 36 of any one of SEQ ID NOs: 1-8; (e) nucleotide 5 through nucleotide 36 of any one of SEQ ID NOs: 1-8; (f) nucleotide 6 through nucleotide 36 of any one of SEQ ID NOs: 1-8; (g) nucleotide 7 through nucleotide 36 of any one of SEQ ID NOs: 1-8; (h) nucleotide 8 through nucleotide 36 of any one of SEQ ID NOs: 1-8; (i) nucleotide 9 through nucleotide 36 of any one of SEQ ID NOs: 1-8; (j) nucleotide 10 through nucleotide 36 of any one of SEQ ID NOs: 1-8; (k) nucleotide 11 through nucleotide 36 of any one of SEQ ID NOs: 1-8; (l) nucleotide 12 through nucleotide 36 of any one of SEQ ID NOs: 1-8; (m) nucleotide 13 through nucleotide 36 of any one of SEQ ID NOs: 1-8; (n) nucleotide 14 through nucleotide 36 of any one of SEQ ID NOs: 1-8; (o) nucleotide 1 through nucleotide 34 of SEQ ID NO: 9; (p) nucleotide 2 through nucleotide 34 of SEQ ID NO: 9; (q) nucleotide 3 through nucleotide 34 of SEQ ID NO: 9; (r) nucleotide 4 through nucleotide 34 of SEQ ID NO: 9; (s) nucleotide 5 through nucleotide 34 of SEQ ID NO: 9; (t) nucleotide 6 through nucleotide 34 of SEQ ID NO: 9; (u) nucleotide 7 through nucleotide 34 of SEQ ID NO: 9; (v) nucleotide 8 through nucleotide 34 of SEQ ID NO: 9; (w) nucleotide 9 through nucleotide 34 of SEQ ID NO: 9; (x) nucleotide 10 through nucleotide 34 of SEQ ID NO: 9; (y) nucleotide 11 through nucleotide 34 of SEQ ID NO: 9; (z) nucleotide 12 through nucleotide 34 of SEQ ID NO: 9; or (aa) SEQ ID NO: 10 or a portion thereof.

In some examples, the direct repeat sequence comprises: (a) nucleotide 1 through nucleotide 36 of a sequence that is at least 90% identical to a sequence of any one of SEQ ID NOs: 936-953; (b) nucleotide 2 through nucleotide 36 of a sequence that is at least 90% identical to a sequence of any one of SEQ ID NOs: 936-953; (c) nucleotide 3 through nucleotide 36 of a sequence that is at least 90% identical to a sequence of any one of SEQ ID NOs: 936-953; (d) nucleotide 4 through nucleotide 36 of a sequence that is at least 90% identical to a sequence of any one of SEQ ID NOs: 936-953; (e) nucleotide 5 through nucleotide 36 of a sequence that is at least 90% identical to a sequence of any one of SEQ ID NOs: 936-953; (f) nucleotide 6 through nucleotide 36 of a sequence that is at least 90% identical to a sequence of any one of SEQ ID NOs: 936-953; (g) nucleotide 7 through nucleotide 36 of a sequence that is at least 90% identical to a sequence of any one of SEQ ID NOs: 936-953; (h) nucleotide 8 through nucleotide 36 of a sequence that is at least 90% identical to a sequence of any one of SEQ ID NOs: 936-953; (i) nucleotide 9 through nucleotide 36 of a sequence that is at least 90% identical to a sequence of any one of SEQ ID NOs: 936-953; (j) nucleotide 10 through nucleotide 36 of a sequence that is at least 90% identical to a sequence of any one of SEQ ID NOs: 936-953; (k) nucleotide 11 through nucleotide 36 of a sequence that is at least 90% identical to a sequence of any one of SEQ ID NOs: 936-953; (l) nucleotide 12 through nucleotide 36 of a sequence that is at least 90% identical to a sequence of any one of SEQ ID NOs: 936-953; (m) nucleotide 13 through nucleotide 36 of a sequence that is at least 90% identical to a sequence of any one of SEQ ID NOs: 936-953; (n) nucleotide 14 through nucleotide 36 of a sequence that is at least 90% identical to a sequence of any one of SEQ ID NOs: 936-953; or (o) a sequence that is at least 90% identical to a sequence of SEQ ID NO: 954 or a portion thereof.

In some examples, the direct repeat sequence comprises: (a) nucleotide 1 through nucleotide 36 of any one of SEQ ID NOs: 936-953; (b) nucleotide 2 through nucleotide 36 of any one of SEQ ID NOs: 936-953; (c) nucleotide 3 through nucleotide 36 of any one of SEQ ID NOs: 936-953; (d) nucleotide 4 through nucleotide 36 of any one of SEQ ID NOs: 936-953; (e) nucleotide 5 through nucleotide 36 of any one of SEQ ID NOs: 936-953; (f) nucleotide 6 through nucleotide 36 of any one of SEQ ID NOs: 936-953; (g) nucleotide 7 through nucleotide 36 of any one of SEQ ID NOs: 936-953; (h) nucleotide 8 through nucleotide 36 of any one of SEQ ID NOs: 936-953; (i) nucleotide 9 through nucleotide 36 of any one of SEQ ID NOs: 936-953; (j) nucleotide 10 through nucleotide 36 of any one of SEQ ID NOs: 936-953; (k) nucleotide 11 through nucleotide 36 of any one of SEQ ID NOs: 936-953; (l) nucleotide 12 through nucleotide 36 of any one of SEQ ID NOs: 936-953; (m) nucleotide 13 through nucleotide 36 of any one of SEQ ID NOs: 936-953; (n) nucleotide 14 through nucleotide 36 of any one of SEQ ID NOs: 936-953; or (o) SEQ ID NO: 954 or a portion thereof.

In some embodiments, the direct repeat sequence comprises: (a) nucleotide 1 through nucleotide 36 of a sequence that is at least 90% identical to SEQ ID NO: 959; (b) nucleotide 2 through nucleotide 36 of a sequence that is at least 90% identical to SEQ ID NO: 959; (c) nucleotide 3 through nucleotide 36 of a sequence that is at least 90% identical to SEQ ID NO: 959; (d) nucleotide 4 through nucleotide 36 of a sequence that is at least 90% identical to SEQ ID NO: 959; (c) nucleotide 5 through nucleotide 36 of a sequence that is at least 90% identical to SEQ ID NO: 959; (f) nucleotide 6 through nucleotide 36 of a sequence that is at least 90% identical to SEQ ID NO: 959; (g) nucleotide 7 through nucleotide 36 of a sequence that is at least 90% identical to SEQ ID NO: 959; (h) nucleotide 8 through nucleotide 36 of a sequence that is at least 90% identical to SEQ ID NO: 959; (i) nucleotide 9 through nucleotide 36 of a sequence that is at least 90% identical to SEQ ID NO: 959; (j) nucleotide 10 through nucleotide 36 of a sequence that is at least 90% identical to SEQ ID NO: 959; (k) nucleotide 11 through nucleotide 36 of a sequence that is at least 90% identical to SEQ ID NO: 959; (l) nucleotide 12 through nucleotide 36 of a sequence that is at least 90% identical to SEQ ID NO: 959; (m) nucleotide 13 through nucleotide 36 of a sequence that is at least 90% identical to SEQ ID NO: 959; (n) nucleotide 14 through nucleotide 36 of a sequence that is at least 90% identical to SEQ ID NO: 959; or (o) a sequence that is at least 90% identical to a sequence of SEQ ID NO: 960 or SEQ ID NO: 961 or a portion thereof.

In some examples, the direct repeat sequence comprises: (a) nucleotide 1 through nucleotide 36 of SEQ ID NO: 959; (b) nucleotide 2 through nucleotide 36 of SEQ ID NO: 959; (c) nucleotide 3 through nucleotide 36 of SEQ ID NO: 959; (d) nucleotide 4 through nucleotide 36 of SEQ ID NO: 959; (c) nucleotide 5 through nucleotide 36 of SEQ ID NO: 959; (f) nucleotide 6 through nucleotide 36 of SEQ ID NO: 959; (g) nucleotide 7 through nucleotide 36 of SEQ ID NO: 959; (h) nucleotide 8 through nucleotide 36 of SEQ ID NO: 959; (i) nucleotide 9 through nucleotide 36 of SEQ ID NO: 959; (j) nucleotide 10 through nucleotide 36 of SEQ ID NO: 959; (k) nucleotide 11 through nucleotide 36 of SEQ ID NO: 959; (l) nucleotide 12 through nucleotide 36 of SEQ ID NO: 959; (m) nucleotide 13 through nucleotide 36 of SEQ ID NO: 959; (n) nucleotide 14 through nucleotide 36 of SEQ ID NO: 959; or (o) SEQ ID NO: 960 or SEQ ID NO: 961 or a portion thereof.

In some examples, the direct repeat sequence comprises: (a) nucleotide 1 through nucleotide 36 of a sequence that is at least 90% identical to a sequence of SEQ ID NO: 962 or SEQ ID NO: 963; (b) nucleotide 2 through nucleotide 36 of a sequence that is at least 90% identical to a sequence of SEQ ID NO: 962 or SEQ ID NO: 963; (c) nucleotide 3 through nucleotide 36 of a sequence that is at least 90% identical to a sequence of SEQ ID NO: 962 or SEQ ID NO: 963; (d) nucleotide 4 through nucleotide 36 of a sequence that is at least 90% identical to a sequence of SEQ ID NO: 962 or SEQ ID NO: 963; (e) nucleotide 5 through nucleotide 36 of a sequence that is at least 90% identical to a sequence of SEQ ID NO: 962 or SEQ ID NO: 963; (f) nucleotide 6 through nucleotide 36 of a sequence that is at least 90% identical to a sequence of SEQ ID NO: 962 or SEQ ID NO: 963; (g) nucleotide 7 through nucleotide 36 of a sequence that is at least 90% identical to a sequence of SEQ ID NO: 962 or SEQ ID NO: 963; (h) nucleotide 8 through nucleotide 36 of a sequence that is at least 90% identical to a sequence of SEQ ID NO: 962 or SEQ ID NO: 963; (i) nucleotide 9 through nucleotide 36 of a sequence that is at least 90% identical to a sequence of SEQ ID NO: 962 or SEQ ID NO: 963; (j) nucleotide 10 through nucleotide 36 of a sequence that is at least 90% identical to a sequence of SEQ ID NO: 962 or SEQ ID NO: 963; (k) nucleotide 11 through nucleotide 36 of a sequence that is at least 90% identical to a sequence of SEQ ID NO: 962 or SEQ ID NO: 963; (l) nucleotide 12 through nucleotide 36 of a sequence that is at least 90% identical to a sequence of SEQ ID NO: 962 or SEQ ID NO: 963; (m) nucleotide 13 through nucleotide 36 of a sequence that is at least 90% identical to a sequence of SEQ ID NO: 962 or SEQ ID NO: 963; (n) nucleotide 14 through nucleotide 36 of a sequence that is at least 90% identical to a sequence of SEQ ID NO: 962 or SEQ ID NO: 963; (o) nucleotide 15 through nucleotide 36 of a sequence that is at least 90% identical to a sequence of SEQ ID NO: 962 or SEQ ID NO: 963; or (p) a sequence that is at least 90% identical to a sequence of SEQ ID NO: 964 or a portion thereof.

In some examples, the direct repeat sequence comprises: (a) nucleotide 1 through nucleotide 36 of SEQ ID NO: 962 or SEQ ID NO: 963; (b) nucleotide 2 through nucleotide 36 of SEQ ID NO: 962 or SEQ ID NO: 963; (c) nucleotide 3 through nucleotide 36 of SEQ ID NO: 962 or SEQ ID NO: 963; (d) nucleotide 4 through nucleotide 36 of SEQ ID NO: 962 or SEQ ID NO: 963; (c) nucleotide 5 through nucleotide 36 of SEQ ID NO: 962 or SEQ ID NO: 963; (f) nucleotide 6 through nucleotide 36 of SEQ ID NO: 962 or SEQ ID NO: 963; (g) nucleotide 7 through nucleotide 36 of SEQ ID NO: 962 or SEQ ID NO: 963; (h) nucleotide 8 through nucleotide 36 of SEQ ID NO: 962 or SEQ ID NO: 963; (i) nucleotide 9 through nucleotide 36 of SEQ ID NO: 962 or SEQ ID NO: 963; (j) nucleotide 10 through nucleotide 36 of SEQ ID NO: 962 or SEQ ID NO: 963; (k) nucleotide 11 through nucleotide 36 of SEQ ID NO: 962 or SEQ ID NO: 963; (l) nucleotide 12 through nucleotide 36 of SEQ ID NO: 962 or SEQ ID NO: 963; (m) nucleotide 13 through nucleotide 36 of SEQ ID NO: 962 or SEQ ID NO: 963; (n) nucleotide 14 through nucleotide 36 of SEQ ID NO: 962 or SEQ ID NO: 963; (o) nucleotide 15 through nucleotide 36 of SEQ ID NO: 962 or SEQ ID NO: 963; or (p) SEQ ID NO: 964 or a portion thereof.

In any of the compositions of Embodiment 4, the spacer sequence may be substantially complementary or completely complementary to the complement of a sequence of any one of SEQ ID NOs: 11-465.

In some examples, the target sequence is adjacent to a protospacer adjacent motif (PAM) comprising the sequence 5′-NTTN-3′. In some examples, the PAM comprises the sequence 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′.

In some examples, the target sequence is immediately adjacent to the PAM sequence. In some examples, the target sequence is within 1, 2, 3, 4, or 5 nucleotides of the PAM sequence. In any of the composition of Embodiment 4, the Cas12i polypeptide is: (a) a Cas12i2 polypeptide comprising a sequence that is at least 90% identical to the sequence of SEQ ID NO: 922, SEQ ID NO: 923, SEQ ID NO: 924, SEQ ID NO: 925, SEQ ID NO: 926, or SEQ ID NO: 927; (b) a Cas12i4 polypeptide comprising a sequence that is at least 90% identical to the sequence of SEQ ID NO: 956, SEQ ID NO: 957, or SEQ ID NO: 958; (c) a Cas12i1 polypeptide comprising a sequence that is at least 90% identical to the sequence of SEQ ID NO: 965; (or (d) a Cas12i3 polypeptide comprising a sequence that is at least 90% identical to the sequence of SEQ ID NO: 966.

In some examples, the Cas12i polypeptide is: (a) a Cas12i2 polypeptide comprising a sequence of SEQ ID NO: 922, SEQ ID NO: 923, SEQ ID NO: 924, SEQ ID NO: 925, SEQ ID NO: 926, or SEQ ID NO: 927; (b) a Cas1214 polypeptide comprising a sequence of SEQ ID NO: 956, SEQ ID NO: 957, or SEQ ID NO: 958; (c) a Cas12i1 polypeptide comprising a sequence of SEQ ID NO: 965; or (d) a Cas12i3 polypeptide comprising a sequence of SEQ ID NO: 966.

In any of the composition of Embodiment 4, the RNA guide and the Cas12i polypeptide may form a ribonucleoprotein complex. In some examples, the ribonucleoprotein complex binds a target nucleic acid.

In any of the composition of Embodiment 4, the composition may be present within a cell.

In any of the composition of Embodiment 4, the RNA guide and the Cas12i polypeptide may be encoded in a vector, e.g., expression vector. In some examples, the RNA guide and the Cas12i polypeptide are encoded in a single vector. In other examples, the RNA guide is encoded in a first vector and the Cas12i polypeptide is encoded in a second vector.

Embodiment 5: A vector system comprising one or more vectors encoding an RNA guide disclosed herein and a Cas12i polypeptide. In some examples, the vector system comprises a first vector encoding an RNA guide disclosed herein and a second vector encoding a Cas12i polypeptide. In some examples, the vectors are expression vectors.

Embodiment 6: An RNA guide comprising (i) a spacer sequence that is substantially complementary or completely complementary to a region on a non-PAM strand (the complementary sequence of a target sequence) within an HAO1 gene, and (ii) a direct repeat sequence.

In some examples, the target sequence is within exon 1, exon 2, exon 3, exon 4, exon 5, exon 6, or exon 7 of the HAO1 gene, which may comprise the sequence of SEQ ID NO: 928, the reverse complement of SEQ ID NO: 928, a variant of the sequence of SEQ ID NO: 928, or the reverse complement of a variant of SEQ ID NO: 928.

In some examples, the spacer sequence comprises: (a) nucleotide 1 through nucleotide 16 of a sequence that is at least 90% identical to a sequence of any one of SEQ ID NOs: 466-920; (b) nucleotide 1 through nucleotide 17 of a sequence that is at least 90% identical to a sequence of any one of SEQ ID NOs: 466-920; (c) nucleotide 1 through nucleotide 18 of a sequence that is at least 90% identical to a sequence of any one of SEQ ID NOs: 466-920; (d) nucleotide 1 through nucleotide 19 of a sequence that is at least 90% identical to a sequence of any one of SEQ ID NOs: 466-920; (c) nucleotide 1 through nucleotide 20 of a sequence that is at least 90% identical to a sequence of any one of SEQ ID NOs: 466-920; (f) nucleotide 1 through nucleotide 21 of a sequence that is at least 90% identical to a sequence of any one of SEQ ID NOs: 466-920; (g) nucleotide 1 through nucleotide 22 of a sequence that is at least 90% identical to a sequence of any one of SEQ ID NOs: 466-920; (h) nucleotide 1 through nucleotide 23 of a sequence that is at least 90% identical to a sequence of any one of SEQ ID NOs: 466-920; (i) nucleotide 1 through nucleotide 24 of a sequence that is at least 90% identical to a sequence of any one of SEQ ID NOs: 466-920; (j) nucleotide 1 through nucleotide 25 of a sequence that is at least 90% identical to a sequence of any one of SEQ ID NOs: 466-920; (k) nucleotide 1 through nucleotide 26 of a sequence that is at least 90% identical to a sequence of any one of SEQ ID NOs: 466-920; (l) nucleotide 1 through nucleotide 27 of a sequence that is at least 90% identical to a sequence of any one of SEQ ID NOs: 466-920; (m) nucleotide 1 through nucleotide 28 of a sequence that is at least 90% identical to a sequence of any one of SEQ ID NOs: 466-920; (n) nucleotide 1 through nucleotide 29 of a sequence that is at least 90% identical to a sequence of any one of SEQ ID NOs: 466-920; or (o) nucleotide 1 through nucleotide 30 of a sequence that is at least 90% identical to a sequence of any one of SEQ ID NOs: 466-920.

In some examples, the spacer sequence comprises: (a) nucleotide 1 through nucleotide 16 of any one of SEQ ID NOs: 466-920; (b) nucleotide 1 through nucleotide 17 of any one of SEQ ID NOs: 466-920; (c) nucleotide 1 through nucleotide 18 of any one of SEQ ID NOs: 466-920; (d) nucleotide 1 through nucleotide 19 of any one of SEQ ID NOs: 466-920; (c) nucleotide 1 through nucleotide 20 of any one of SEQ ID NOs: 466-920; (f) nucleotide 1 through nucleotide 21 of any one of SEQ ID NOs: 466-920; (g) nucleotide 1 through nucleotide 22 of any one of SEQ ID NOs: 466-920; (h) nucleotide 1 through nucleotide 23 of any one of SEQ ID NOs: 466-920; (i) nucleotide 1 through nucleotide 24 of any one of SEQ ID NOs: 466-920; (j) nucleotide 1 through nucleotide 25 of any one of SEQ ID NOs: 466-920; (k) nucleotide 1 through nucleotide 26 of any one of SEQ ID NOs: 466-920; (l) nucleotide 1 through nucleotide 27 of any one of SEQ ID NOs: 466-920; (m) nucleotide 1 through nucleotide 28 of any one of SEQ ID NOs: 466-920; (n) nucleotide 1 through nucleotide 29 of any one of SEQ ID NOs: 466-920; or (o) nucleotide 1 through nucleotide 30 of any one of SEQ ID NOs: 466-920.

In some examples, the direct repeat sequence comprises: (a) nucleotide 1 through nucleotide 36 of a sequence that is at least 90% identical to a sequence of any one of SEQ ID NOs: 1-8; (b) nucleotide 2 through nucleotide 36 of a sequence that is at least 90% identical to a sequence of any one of SEQ ID NOs: 1-8; (c) nucleotide 3 through nucleotide 36 of a sequence that is at least 90% identical to a sequence of any one of SEQ ID NOs: 1-8; (d) nucleotide 4 through nucleotide 36 of a sequence that is at least 90% identical to a sequence of any one of SEQ ID NOs: 1-8; (e) nucleotide 5 through nucleotide 36 of a sequence that is at least 90% identical to a sequence of any one of SEQ ID NOs: 1-8; (f) nucleotide 6 through nucleotide 36 of a sequence that is at least 90% identical to a sequence of any one of SEQ ID NOs: 1-8; (g) nucleotide 7 through nucleotide 36 of a sequence that is at least 90% identical to a sequence of any one of SEQ ID NOs: 1-8; (h) nucleotide 8 through nucleotide 36 of a sequence that is at least 90% identical to a sequence of any one of SEQ ID NOs: 1-8; (i) nucleotide 9 through nucleotide 36 of a sequence that is at least 90% identical to a sequence of any one of SEQ ID NOs: 1-8; (j) nucleotide 10 through nucleotide 36 of a sequence that is at least 90% identical to a sequence of any one of SEQ ID NOs: 1-8; (k) nucleotide 11 through nucleotide 36 of a sequence that is at least 90% identical to a sequence of any one of SEQ ID NOs: 1-8; (l) nucleotide 12 through nucleotide 36 of a sequence that is at least 90% identical to a sequence of any one of SEQ ID NOs: 1-8; (m) nucleotide 13 through nucleotide 36 of a sequence that is at least 90% identical to a sequence of any one of SEQ ID NOs: 1-8; (n) nucleotide 14 through nucleotide 36 of a sequence that is at least 90% identical to a sequence of any one of SEQ ID NOs: 1-8; (o) nucleotide 1 through nucleotide 34 of a sequence that is at least 90% identical to a sequence of SEQ ID NO: 9; (p) nucleotide 2 through nucleotide 34 of a sequence that is at least 90% identical to a sequence of SEQ ID NO: 9; (q) nucleotide 3 through nucleotide 34 of a sequence that is at least 90% identical to a sequence of SEQ ID NO: 9; (r) nucleotide 4 through nucleotide 34 of a sequence that is at least 90% identical to a sequence of SEQ ID NO: 9; (s) nucleotide 5 through nucleotide 34 of a sequence that is at least 90% identical to a sequence of SEQ ID NO: 9; (t) nucleotide 6 through nucleotide 34 of a sequence that is at least 90% identical to a sequence of SEQ ID NO: 9; (u) nucleotide 7 through nucleotide 34 of a sequence that is at least 90% identical to a sequence of SEQ ID NO: 9; (v) nucleotide 8 through nucleotide 34 of a sequence that is at least 90% identical to a sequence of SEQ ID NO: 9; (w) nucleotide 9 through nucleotide 34 of a sequence that is at least 90% identical to a sequence of SEQ ID NO: 9; (x) nucleotide 10 through nucleotide 34 of a sequence that is at least 90% identical to a sequence of SEQ ID NO: 9; (y) nucleotide 11 through nucleotide 34 of a sequence that is at least 90% identical to a sequence of SEQ ID NO: 9; (z) nucleotide 12 through nucleotide 34 of a sequence that is at least 90% identical to a sequence of SEQ ID NO: 9; or (aa) a sequence that is at least 90% identical to a sequence of SEQ ID NO: 10 or a portion thereof.

In some examples, the direct repeat sequence comprises: (a) nucleotide 1 through nucleotide 36 of any one of SEQ ID NOs: 1-8; (b) nucleotide 2 through nucleotide 36 of any one of SEQ ID NOs: 1-8; (c) nucleotide 3 through nucleotide 36 of any one of SEQ ID NOs: 1-8; (d) nucleotide 4 through nucleotide 36 of any one of SEQ ID NOs: 1-8; (e) nucleotide 5 through nucleotide 36 of any one of SEQ ID NOs: 1-8; (f) nucleotide 6 through nucleotide 36 of any one of SEQ ID NOs: 1-8; (g) nucleotide 7 through nucleotide 36 of any one of SEQ ID NOs: 1-8; (h) nucleotide 8 through nucleotide 36 of any one of SEQ ID NOs: 1-8; (i) nucleotide 9 through nucleotide 36 of any one of SEQ ID NOs: 1-8; (j) nucleotide 10 through nucleotide 36 of any one of SEQ ID NOs: 1-8; (k) nucleotide 11 through nucleotide 36 of any one of SEQ ID NOs: 1-8; (l) nucleotide 12 through nucleotide 36 of any one of SEQ ID NOs: 1-8; (m) nucleotide 13 through nucleotide 36 of any one of SEQ ID NOs: 1-8; (n) nucleotide 14 through nucleotide 36 of any one of SEQ ID NOs: 1-8; (o) nucleotide 1 through nucleotide 34 of SEQ ID NO: 9; (p) nucleotide 2 through nucleotide 34 of SEQ ID NO: 9; (q) nucleotide 3 through nucleotide 34 of SEQ ID NO: 9; (r) nucleotide 4 through nucleotide 34 of SEQ ID NO: 9; (s) nucleotide 5 through nucleotide 34 of SEQ ID NO: 9; (t) nucleotide 6 through nucleotide 34 of SEQ ID NO: 9; (u) nucleotide 7 through nucleotide 34 of SEQ ID NO: 9; (v) nucleotide 8 through nucleotide 34 of SEQ ID NO: 9; (w) nucleotide 9 through nucleotide 34 of SEQ ID NO: 9; (x) nucleotide 10 through nucleotide 34 of SEQ ID NO: 9; (y) nucleotide 11 through nucleotide 34 of SEQ ID NO: 9; (z) nucleotide 12 through nucleotide 34 of SEQ ID NO: 9; or (aa) SEQ ID NO: 10 or a portion thereof.

In some examples, the direct repeat sequence comprises: (a) nucleotide 1 through nucleotide 36 of a sequence that is at least 90% identical to a sequence of any one of SEQ ID NOs: 936-953; (b) nucleotide 2 through nucleotide 36 of a sequence that is at least 90% identical to a sequence of any one of SEQ ID NOs: 936-953; (c) nucleotide 3 through nucleotide 36 of a sequence that is at least 90% identical to a sequence of any one of SEQ ID NOs: 936-953; (d) nucleotide 4 through nucleotide 36 of a sequence that is at least 90% identical to a sequence of any one of SEQ ID NOs: 936-953; (c) nucleotide 5 through nucleotide 36 of a sequence that is at least 90% identical to a sequence of any one of SEQ ID NOs: 936-953; (f) nucleotide 6 through nucleotide 36 of a sequence that is at least 90% identical to a sequence of any one of SEQ ID NOs: 936-953; (g) nucleotide 7 through nucleotide 36 of a sequence that is at least 90% identical to a sequence of any one of SEQ ID NOs: 936-953; (h) nucleotide 8 through nucleotide 36 of a sequence that is at least 90% identical to a sequence of any one of SEQ ID NOs: 936-953; (i) nucleotide 9 through nucleotide 36 of a sequence that is at least 90% identical to a sequence of any one of SEQ ID NOs: 936-953; (j) nucleotide 10 through nucleotide 36 of a sequence that is at least 90% identical to a sequence of any one of SEQ ID NOs: 936-953; (k) nucleotide 11 through nucleotide 36 of a sequence that is at least 90% identical to a sequence of any one of SEQ ID NOs: 936-953; (l) nucleotide 12 through nucleotide 36 of a sequence that is at least 90% identical to a sequence of any one of SEQ ID NOs: 936-953; (m) nucleotide 13 through nucleotide 36 of a sequence that is at least 90% identical to a sequence of any one of SEQ ID NOs: 936-953; (n) nucleotide 14 through nucleotide 36 of a sequence that is at least 90% identical to a sequence of any one of SEQ ID NOs: 936-953; or (o) a sequence that is at least 90% identical to a sequence of SEQ ID NO: 954 or a portion thereof.

In some examples, the direct repeat sequence comprises: (a) nucleotide 1 through nucleotide 36 of any one of SEQ ID NOs: 936-953; (b) nucleotide 2 through nucleotide 36 of any one of SEQ ID NOs: 936-953; (c) nucleotide 3 through nucleotide 36 of any one of SEQ ID NOs: 936-953; (d) nucleotide 4 through nucleotide 36 of any one of SEQ ID NOs: 936-953; (e) nucleotide 5 through nucleotide 36 of any one of SEQ ID NOs: 936-953; (f) nucleotide 6 through nucleotide 36 of any one of SEQ ID NOs: 936-953; (g) nucleotide 7 through nucleotide 36 of any one of SEQ ID NOs: 936-953; (h) nucleotide 8 through nucleotide 36 of any one of SEQ ID NOs: 936-953; (i) nucleotide 9 through nucleotide 36 of any one of SEQ ID NOs: 936-953; (j) nucleotide 10 through nucleotide 36 of any one of SEQ ID NOs: 936-953; (k) nucleotide 11 through nucleotide 36 of any one of SEQ ID NOs: 936-953; (l) nucleotide 12 through nucleotide 36 of any one of SEQ ID NOs: 936-953; (m) nucleotide 13 through nucleotide 36 of any one of SEQ ID NOs: 936-953; (n) nucleotide 14 through nucleotide 36 of any one of SEQ ID NOs: 936-953; or (o) SEQ ID NO: 954 or a portion thereof.

In some examples, the direct repeat sequence comprises: (a) nucleotide 1 through nucleotide 36 of a sequence that is at least 90% identical to SEQ ID NO: 959; (b) nucleotide 2 through nucleotide 36 of a sequence that is at least 90% identical to SEQ ID NO: 959; (c) nucleotide 3 through nucleotide 36 of a sequence that is at least 90% identical to SEQ ID NO: 959; (d) nucleotide 4 through nucleotide 36 of a sequence that is at least 90% identical to SEQ ID NO: 959; (e) nucleotide 5 through nucleotide 36 of a sequence that is at least 90% identical to SEQ ID NO: 959; (f) nucleotide 6 through nucleotide 36 of a sequence that is at least 90% identical to SEQ ID NO: 959; (g) nucleotide 7 through nucleotide 36 of a sequence that is at least 90% identical to SEQ ID NO: 959; (h) nucleotide 8 through nucleotide 36 of a sequence that is at least 90% identical to SEQ ID NO: 959; (i) nucleotide 9 through nucleotide 36 of a sequence that is at least 90% identical to SEQ ID NO: 959; (j) nucleotide 10 through nucleotide 36 of a sequence that is at least 90% identical to SEQ ID NO: 959; (k) nucleotide 11 through nucleotide 36 of a sequence that is at least 90% identical to SEQ ID NO: 959; (l) nucleotide 12 through nucleotide 36 of a sequence that is at least 90% identical to SEQ ID NO: 959; (m) nucleotide 13 through nucleotide 36 of a sequence that is at least 90% identical to SEQ ID NO: 959; (n) nucleotide 14 through nucleotide 36 of a sequence that is at least 90% identical to SEQ ID NO: 959; or (o) a sequence that is at least 90% identical to a sequence of SEQ ID NO: 960 or SEQ ID NO: 961 or a portion thereof.

In some examples, the direct repeat sequence comprises: (a) nucleotide 1 through nucleotide 36 of SEQ ID NO: 959; (b) nucleotide 2 through nucleotide 36 of SEQ ID NO: 959; (c) nucleotide 3 through nucleotide 36 of SEQ ID NO: 959; (d) nucleotide 4 through nucleotide 36 of SEQ ID NO: 959; (c) nucleotide 5 through nucleotide 36 of SEQ ID NO: 959; (f) nucleotide 6 through nucleotide 36 of SEQ ID NO: 959; (g) nucleotide 7 through nucleotide 36 of SEQ ID NO: 959; (h) nucleotide 8 through nucleotide 36 of SEQ ID NO: 959; (i) nucleotide 9 through nucleotide 36 of SEQ ID NO: 959; (j) nucleotide 10 through nucleotide 36 of SEQ ID NO: 959; (k) nucleotide 11 through nucleotide 36 of SEQ ID NO: 959; (l) nucleotide 12 through nucleotide 36 of SEQ ID NO: 959; (m) nucleotide 13 through nucleotide 36 of SEQ ID NO: 959; (n) nucleotide 14 through nucleotide 36 of SEQ ID NO: 959; or (o) SEQ ID NO: 960 or SEQ ID NO: 961 or a portion thereof.

In some examples, the direct repeat sequence comprises: (a) nucleotide 1 through nucleotide 36 of a sequence that is at least 90% identical to a sequence of SEQ ID NO: 962 or SEQ ID NO: 963; (b) nucleotide 2 through nucleotide 36 of a sequence that is at least 90% identical to a sequence of SEQ ID NO: 962 or SEQ ID NO: 963; (c) nucleotide 3 through nucleotide 36 of a sequence that is at least 90% identical to a sequence of SEQ ID NO: 962 or SEQ ID NO: 963; (d) nucleotide 4 through nucleotide 36 of a sequence that is at least 90% identical to a sequence of SEQ ID NO: 962 or SEQ ID NO: 963; (e) nucleotide 5 through nucleotide 36 of a sequence that is at least 90% identical to a sequence of SEQ ID NO: 962 or SEQ ID NO: 963; (f) nucleotide 6 through nucleotide 36 of a sequence that is at least 90% identical to a sequence of SEQ ID NO: 962 or SEQ ID NO: 963; (g) nucleotide 7 through nucleotide 36 of a sequence that is at least 90% identical to a sequence of SEQ ID NO: 962 or SEQ ID NO: 963; (h) nucleotide 8 through nucleotide 36 of a sequence that is at least 90% identical to a sequence of SEQ ID NO: 962 or SEQ ID NO: 963; (i) nucleotide 9 through nucleotide 36 of a sequence that is at least 90% identical to a sequence of SEQ ID NO: 962 or SEQ ID NO: 963; (j) nucleotide 10 through nucleotide 36 of a sequence that is at least 90% identical to a sequence of SEQ ID NO: 962 or SEQ ID NO: 963; (k) nucleotide 11 through nucleotide 36 of a sequence that is at least 90% identical to a sequence of SEQ ID NO: 962 or SEQ ID NO: 963; (l) nucleotide 12 through nucleotide 36 of a sequence that is at least 90% identical to a sequence of SEQ ID NO: 962 or SEQ ID NO: 963; (m) nucleotide 13 through nucleotide 36 of a sequence that is at least 90% identical to a sequence of SEQ ID NO: 962 or SEQ ID NO: 963; (n) nucleotide 14 through nucleotide 36 of a sequence that is at least 90% identical to a sequence of SEQ ID NO: 962 or SEQ ID NO: 963; (o) nucleotide 15 through nucleotide 36 of a sequence that is at least 90% identical to a sequence of SEQ ID NO: 962 or SEQ ID NO: 963; or (p) a sequence that is at least 90% identical to a sequence of SEQ ID NO: 964 or a portion thereof.

In some examples, the direct repeat sequence comprises: (a) nucleotide 1 through nucleotide 36 of SEQ ID NO: 962 or SEQ ID NO: 963; (b) nucleotide 2 through nucleotide 36 of SEQ ID NO: 962 or SEQ ID NO: 963; (c) nucleotide 3 through nucleotide 36 of SEQ ID NO: 962 or SEQ ID NO: 963; (d) nucleotide 4 through nucleotide 36 of SEQ ID NO: 962 or SEQ ID NO: 963; (c) nucleotide 5 through nucleotide 36 of SEQ ID NO: 962 or SEQ ID NO: 963; (f) nucleotide 6 through nucleotide 36 of SEQ ID NO: 962 or SEQ ID NO: 963; (g) nucleotide 7 through nucleotide 36 of SEQ ID NO: 962 or SEQ ID NO: 963; (h) nucleotide 8 through nucleotide 36 of SEQ ID NO: 962 or SEQ ID NO: 963; (i) nucleotide 9 through nucleotide 36 of SEQ ID NO: 962 or SEQ ID NO: 963; (j) nucleotide 10 through nucleotide 36 of SEQ ID NO: 962 or SEQ ID NO: 963; (k) nucleotide 11 through nucleotide 36 of SEQ ID NO: 962 or SEQ ID NO: 963; (l) nucleotide 12 through nucleotide 36 of SEQ ID NO: 962 or SEQ ID NO: 963; (m) nucleotide 13 through nucleotide 36 of SEQ ID NO: 962 or SEQ ID NO: 963; (n) nucleotide 14 through nucleotide 36 of SEQ ID NO: 962 or SEQ ID NO: 963; (o) nucleotide 15 through nucleotide 36 of SEQ ID NO: 962 or SEQ ID NO: 963; or (p) SEQ ID NO: 964 or a portion thereof.

In any of the RNA guide of Embodiment 6, the spacer sequence may be substantially complementary or completely complementary to the complement of a sequence of any one of SEQ ID NOs: 11-465.

In any of the RNA guide of Embodiment 6, the target sequence may be adjacent to a protospacer adjacent motif (PAM) comprising the sequence 5′-NTTN-3′, wherein N is any nucleotide. In some examples, the PAM comprises the sequence 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′.

In some examples, the target sequence is immediately adjacent to the PAM sequence. In other examples, the target sequence is within 1, 2, 3, 4, or 5 nucleotides of the PAM sequence.

In some examples, the RNA guide has a sequence that is at least 90% identical to a sequence of any one of SEQ ID NOs: 967-1023. In some specific examples, the RNA guide has the sequence of any one of SEQ ID NOs: 967-1023.

Embodiment 7: A nucleic acid encoding an RNA guide as described herein.

Embodiment 8: A vector comprising an RNA guide as described herein.

Embodiment 9: A cell comprising a composition, an RNA guide, a nucleic acid, or a vector as described herein. In some examples, the cell is a eukaryotic cell, an animal cell, a mammalian cell, a human cell, a primary cell, a cell line, a stem cell, or a hepatocyte.

Embodiment 10: A kit comprising a composition, an RNA guide, a nucleic acid, or a vector as described herein.

Embodiment 11: A method of editing an HAO1 sequence, the method comprising contacting an HAO1 sequence with a composition or an RNA guide as described herein. In some examples, the method is carried out in vitro. In other examples, the method is carried out ex vivo.

In some examples, the HAO1 sequence is in a cell.

In some examples, the composition or the RNA guide induces a deletion in the HAO1 sequence. In some examples, the deletion is adjacent to a 5′-NTTN-3′ sequence, wherein N is any nucleotide. In some specific examples, the deletion is downstream of the 5′-NTTN-3′ sequence. In some specific examples, the deletion is up to about 40 nucleotides in length. In some instances, the deletion is from about 4 nucleotides to 40 nucleotides, about 4 nucleotides to 25 nucleotides, about 10 nucleotides to 25 nucleotides, or about 10 nucleotides to 15 nucleotides in length.

In some examples, the deletion starts within about 5 nucleotides to about 15 nucleotides, about 5 nucleotides to about 10 nucleotides, or about 10 nucleotides to about 15 nucleotides of the 5′-NTTN-3′ sequence.

In some examples, the deletion starts within about 5 nucleotides to about 15 nucleotides, about 5 nucleotides to about 10 nucleotides, or about 10 nucleotides to about 15 nucleotides downstream of the 5′-NTTN-3′ sequence.

In some examples, the deletion ends within about 20 nucleotides to about 30 nucleotides, about 20 nucleotides to about 25 nucleotides, or about 25 nucleotides to about 30 nucleotides of the 5′-NTTN-3′ sequence.

In some examples, the deletion ends within about 20 nucleotides to about 30 nucleotides, about 20 nucleotides to about 25 nucleotides, about 25 nucleotides to about 30 nucleotides downstream of the 5′-NTTN-3′ sequence.

In some examples, the deletion starts within about 5 nucleotides to about 15 nucleotides downstream of the 5′-NTTN-3′ sequence and ends within about 20 nucleotides to about 30 nucleotides downstream of the 5′-NTTN-3′ sequence.

In some examples, the deletion starts within about 5 nucleotides to about 15 nucleotides downstream of the 5′-NTTN-3′ sequence and ends within about 20 nucleotides to about 25 nucleotides downstream of the 5′-NTTN-3′ sequence.

In some examples, the deletion starts within about 5 nucleotides to about 15 nucleotides downstream of the 5′-NTTN-3′ sequence and ends within about 25 nucleotides to about 30 nucleotides downstream of the 5′-NTTN-3′ sequence.

In some examples, the deletion starts within about 5 nucleotides to about 10 nucleotides downstream of the 5′-NTTN-3′ sequence and ends within about 20 nucleotides to about 30 nucleotides downstream of the 5′-NTTN-3′ sequence.

In some examples, the deletion starts within about 5 nucleotides to about 10 nucleotides downstream of the 5′-NTTN-3′ sequence and ends within about 20 nucleotides to about 25 nucleotides downstream of the 5′-NTTN-3′ sequence.

In some examples, the deletion starts within about 5 nucleotides to about 10 nucleotides downstream of the 5′-NTTN-3′ sequence and ends within about 25 nucleotides to about 30 nucleotides downstream of the 5′-NTTN-3′ sequence.

In some examples, the deletion starts within about 10 nucleotides to about 15 nucleotides downstream of the 5′-NTTN-3′ sequence and ends within about 20 nucleotides to about 30 nucleotides downstream of the 5′-NTTN-3′ sequence.

In some examples, the deletion starts within about 10 nucleotides to about 15 nucleotides downstream of the 5′-NTTN-3′ sequence and ends within about 20 nucleotides to about 25 nucleotides downstream of the 5′-NTTN-3′ sequence.

In some examples, the deletion starts within about 10 nucleotides to about 15 nucleotides downstream of the 5′-NTTN-3′ sequence and ends within about 25 nucleotides to about 30 nucleotides downstream of the 5′-NTTN-3′ sequence.

In some examples, the 5′-NTTN-3′ sequence is 5′-CTTT-3′, 5′-CTTC-3′, 5′-GTTT-3′, 5′-GTTC-3′, 5′-TTTC-3′, 5′-GTTA-3′, or 5′-GTTG-3′.

In some examples, the deletion overlaps with a mutation in the HAO1 sequence. In some instances, the deletion overlaps with an insertion in the HAO1 sequence. In some instances, the deletion removes a repeat expansion of the HAO1 sequence or a portion thereof. In some instances, the deletion disrupts one or both alleles of the HAO1 sequence.

In any of the compositions, RNA guides, nucleic acids, vectors, cells, kits, or methods of Embodiments 1-10 described herein, the RNA guide may comprise the sequence of any one of SEQ ID NOs: 967-1023.

Embodiment 12: A method of treating primary hyperoxaluria (PH), which optionally is PH1, PH2, or PH3, in a subject, the method comprising administering any of the compositions, RNAs, or cells as described herein to the subject.

In any of the compositions, RNA guides, cells, kits, or methods described herein, the RNA guide and/or the polyribonucleotide encoding the Cas12i polypeptide may be comprised within a lipid nanoparticle. In some examples, the RNA guide and the polyribonucleotide encoding the Cas12i polypeptide are comprised within the same lipid nanoparticle. In other examples, the RNA guide and the polyribonucleotide encoding the Cas12i polypeptide are comprised within separate lipid nanoparticles.

Embodiment 13: An RNA guide comprising (i) a spacer sequence that is complementary to a target site within an HAO1 gene (the target site being on the non-PAM strand and complementary to a target sequence), and (ii) a direct repeat sequence, wherein the target sequence is any one of SEQ ID NOs: 1047, 1026, or 1025 or the reverse complement thereof. In some examples, the direct repeat sequence comprises: (a) nucleotide 1 through nucleotide 36 of a sequence that is at least 90% identical to a sequence of any one of SEQ ID NOs: 1-8; (b) nucleotide 2 through nucleotide 36 of a sequence that is at least 90% identical to a sequence of any one of SEQ ID NOs: 1-8; (c) nucleotide 3 through nucleotide 36 of a sequence that is at least 90% identical to a sequence of any one of SEQ ID NOs: 1-8; (d) nucleotide 4 through nucleotide 36 of a sequence that is at least 90% identical to a sequence of any one of SEQ ID NOs: 1-8; (c) nucleotide 5 through nucleotide 36 of a sequence that is at least 90% identical to a sequence of any one of SEQ ID NOs: 1-8; (f) nucleotide 6 through nucleotide 36 of a sequence that is at least 90% identical to a sequence of any one of SEQ ID NOs: 1-8; (g) nucleotide 7 through nucleotide 36 of a sequence that is at least 90% identical to a sequence of any one of SEQ ID NOs: 1-8; (h) nucleotide 8 through nucleotide 36 of a sequence that is at least 90% identical to a sequence of any one of SEQ ID NOs: 1-8; (i) nucleotide 9 through nucleotide 36 of a sequence that is at least 90% identical to a sequence of any one of SEQ ID NOs: 1-8; (j) nucleotide 10 through nucleotide 36 of a sequence that is at least 90% identical to a sequence of any one of SEQ ID NOs: 1-8; (k) nucleotide 11 through nucleotide 36 of a sequence that is at least 90% identical to a sequence of any one of SEQ ID NOs: 1-8; (l) nucleotide 12 through nucleotide 36 of a sequence that is at least 90% identical to a sequence of any one of SEQ ID NOs: 1-8; (m) nucleotide 13 through nucleotide 36 of a sequence that is at least 90% identical to a sequence of any one of SEQ ID NOs: 1-8; (n) nucleotide 14 through nucleotide 36 of a sequence that is at least 90% identical to a sequence of any one of SEQ ID NOs: 1-8; (o) nucleotide 1 through nucleotide 34 of a sequence that is at least 90% identical to a sequence of SEQ ID NO: 9; (p) nucleotide 2 through nucleotide 34 of a sequence that is at least 90% identical to a sequence of SEQ ID NO: 9; (q) nucleotide 3 through nucleotide 34 of a sequence that is at least 90% identical to a sequence of SEQ ID NO: 9; (r) nucleotide 4 through nucleotide 34 of a sequence that is at least 90% identical to a sequence of SEQ ID NO: 9; (s) nucleotide 5 through nucleotide 34 of a sequence that is at least 90% identical to a sequence of SEQ ID NO: 9; (t) nucleotide 6 through nucleotide 34 of a sequence that is at least 90% identical to a sequence of SEQ ID NO: 9; (u) nucleotide 7 through nucleotide 34 of a sequence that is at least 90% identical to a sequence of SEQ ID NO: 9; (v) nucleotide 8 through nucleotide 34 of a sequence that is at least 90% identical to a sequence of SEQ ID NO: 9; (w) nucleotide 9 through nucleotide 34 of a sequence that is at least 90% identical to a sequence of SEQ ID NO: 9; (x) nucleotide 10 through nucleotide 34 of a sequence that is at least 90% identical to a sequence of SEQ ID NO: 9; (y) nucleotide 11 through nucleotide 34 of a sequence that is at least 90% identical to a sequence of SEQ ID NO: 9; (z) nucleotide 12 through nucleotide 34 of a sequence that is at least 90% identical to a sequence of SEQ ID NO: 9; or (aa) a sequence that is at least 90% identical to a sequence of SEQ ID NO: 10 or a portion thereof.

In some examples, the direct repeat sequence comprises: (a) nucleotide 1 through nucleotide 36 of any one of SEQ ID NOs: 1-8; (b) nucleotide 2 through nucleotide 36 of any one of SEQ ID NOs: 1-8; (c) nucleotide 3 through nucleotide 36 of any one of SEQ ID NOs: 1-8; (d) nucleotide 4 through nucleotide 36 of any one of SEQ ID NOs: 1-8; (e) nucleotide 5 through nucleotide 36 of any one of SEQ ID NOs: 1-8; (f) nucleotide 6 through nucleotide 36 of any one of SEQ ID NOs: 1-8; (g) nucleotide 7 through nucleotide 36 of any one of SEQ ID NOs: 1-8; (h) nucleotide 8 through nucleotide 36 of any one of SEQ ID NOs: 1-8; (i) nucleotide 9 through nucleotide 36 of any one of SEQ ID NOs: 1-8; (j) nucleotide 10 through nucleotide 36 of any one of SEQ ID NOs: 1-8; (k) nucleotide 11 through nucleotide 36 of any one of SEQ ID NOs: 1-8; (l) nucleotide 12 through nucleotide 36 of any one of SEQ ID NOs: 1-8; (m) nucleotide 13 through nucleotide 36 of any one of SEQ ID NOs: 1-8; (n) nucleotide 14 through nucleotide 36 of any one of SEQ ID NOs: 1-8; (o) nucleotide 1 through nucleotide 34 of SEQ ID NO: 9; (p) nucleotide 2 through nucleotide 34 of SEQ ID NO: 9; (q) nucleotide 3 through nucleotide 34 of SEQ ID NO: 9; (r) nucleotide 4 through nucleotide 34 of SEQ ID NO: 9; (s) nucleotide 5 through nucleotide 34 of SEQ ID NO: 9; (t) nucleotide 6 through nucleotide 34 of SEQ ID NO: 9; (u) nucleotide 7 through nucleotide 34 of SEQ ID NO: 9; (v) nucleotide 8 through nucleotide 34 of SEQ ID NO: 9; (w) nucleotide 9 through nucleotide 34 of SEQ ID NO: 9; (x) nucleotide 10 through nucleotide 34 of SEQ ID NO: 9; (y) nucleotide 11 through nucleotide 34 of SEQ ID NO: 9; (z) nucleotide 12 through nucleotide 34 of SEQ ID NO: 9; or (aa) SEQ ID NO: 10 or a portion thereof.

In some examples, the direct repeat sequence comprises: (a) nucleotide 1 through nucleotide 36 of a sequence that is at least 90% identical to a sequence of any one of SEQ ID NOs: 936-953; (b) nucleotide 2 through nucleotide 36 of a sequence that is at least 90% identical to a sequence of any one of SEQ ID NOs: 936-953; (c) nucleotide 3 through nucleotide 36 of a sequence that is at least 90% identical to a sequence of any one of SEQ ID NOs: 936-953; (d) nucleotide 4 through nucleotide 36 of a sequence that is at least 90% identical to a sequence of any one of SEQ ID NOs: 936-953; (e) nucleotide 5 through nucleotide 36 of a sequence that is at least 90% identical to a sequence of any one of SEQ ID NOs: 936-953; (f) nucleotide 6 through nucleotide 36 of a sequence that is at least 90% identical to a sequence of any one of SEQ ID NOs: 936-953; (g) nucleotide 7 through nucleotide 36 of a sequence that is at least 90% identical to a sequence of any one of SEQ ID NOs: 936-953; (h) nucleotide 8 through nucleotide 36 of a sequence that is at least 90% identical to a sequence of any one of SEQ ID NOs: 936-953; (i) nucleotide 9 through nucleotide 36 of a sequence that is at least 90% identical to a sequence of any one of SEQ ID NOs: 936-953; (j) nucleotide 10 through nucleotide 36 of a sequence that is at least 90% identical to a sequence of any one of SEQ ID NOs: 936-953; (k) nucleotide 11 through nucleotide 36 of a sequence that is at least 90% identical to a sequence of any one of SEQ ID NOs: 936-953; (l) nucleotide 12 through nucleotide 36 of a sequence that is at least 90% identical to a sequence of any one of SEQ ID NOs: 936-953; (m) nucleotide 13 through nucleotide 36 of a sequence that is at least 90% identical to a sequence of any one of SEQ ID NOs: 936-953; (n) nucleotide 14 through nucleotide 36 of a sequence that is at least 90% identical to a sequence of any one of SEQ ID NOs: 936-953; or (o) a sequence that is at least 90% identical to a sequence of SEQ ID NO: 954 or a portion thereof.

In some examples, the direct repeat sequence comprises: (a) nucleotide 1 through nucleotide 36 of any one of SEQ ID NOs: 936-953; (b) nucleotide 2 through nucleotide 36 of any one of SEQ ID NOs: 936-953; (c) nucleotide 3 through nucleotide 36 of any one of SEQ ID NOs: 936-953; (d) nucleotide 4 through nucleotide 36 of any one of SEQ ID NOs: 936-953; (e) nucleotide 5 through nucleotide 36 of any one of SEQ ID NOs: 936-953; (f) nucleotide 6 through nucleotide 36 of any one of SEQ ID NOs: 936-953; (g) nucleotide 7 through nucleotide 36 of any one of SEQ ID NOs: 936-953; (h) nucleotide 8 through nucleotide 36 of any one of SEQ ID NOs: 936-953; (i) nucleotide 9 through nucleotide 36 of any one of SEQ ID NOs: 936-953; (j) nucleotide 10 through nucleotide 36 of any one of SEQ ID NOs: 936-953; (k) nucleotide 11 through nucleotide 36 of any one of SEQ ID NOs: 936-953; (l) nucleotide 12 through nucleotide 36 of any one of SEQ ID NOs: 936-953; (m) nucleotide 13 through nucleotide 36 of any one of SEQ ID NOs: 936-953; (n) nucleotide 14 through nucleotide 36 of any one of SEQ ID NOs: 936-953; or (o) SEQ ID NO: 954 or a portion thereof.

In some examples, the direct repeat sequence comprises: (a) nucleotide 1 through nucleotide 36 of a sequence that is at least 90% identical to SEQ ID NO: 959; (b) nucleotide 2 through nucleotide 36 of a sequence that is at least 90% identical to SEQ ID NO: 959; (c) nucleotide 3 through nucleotide 36 of a sequence that is at least 90% identical to SEQ ID NO: 959; (d) nucleotide 4 through nucleotide 36 of a sequence that is at least 90% identical to SEQ ID NO: 959; (e) nucleotide 5 through nucleotide 36 of a sequence that is at least 90% identical to SEQ ID NO: 959; (f) nucleotide 6 through nucleotide 36 of a sequence that is at least 90% identical to SEQ ID NO: 959; (g) nucleotide 7 through nucleotide 36 of a sequence that is at least 90% identical to SEQ ID NO: 959; (h) nucleotide 8 through nucleotide 36 of a sequence that is at least 90% identical to SEQ ID NO: 959; (i) nucleotide 9 through nucleotide 36 of a sequence that is at least 90% identical to SEQ ID NO: 959; (j) nucleotide 10 through nucleotide 36 of a sequence that is at least 90% identical to SEQ ID NO: 959; (k) nucleotide 11 through nucleotide 36 of a sequence that is at least 90% identical to SEQ ID NO: 959; (l) nucleotide 12 through nucleotide 36 of a sequence that is at least 90% identical to SEQ ID NO: 959; (m) nucleotide 13 through nucleotide 36 of a sequence that is at least 90% identical to SEQ ID NO: 959; (n) nucleotide 14 through nucleotide 36 of a sequence that is at least 90% identical to SEQ ID NO: 959; or (o) a sequence that is at least 90% identical to a sequence of SEQ ID NO: 960 or SEQ ID NO: 961 or a portion thereof.

In some examples, the direct repeat sequence comprises: (a) nucleotide 1 through nucleotide 36 of SEQ ID NO: 959; (b) nucleotide 2 through nucleotide 36 of SEQ ID NO: 959; (c) nucleotide 3 through nucleotide 36 of SEQ ID NO: 959; (d) nucleotide 4 through nucleotide 36 of SEQ ID NO: 959; (c) nucleotide 5 through nucleotide 36 of SEQ ID NO: 959; (f) nucleotide 6 through nucleotide 36 of SEQ ID NO: 959; (g) nucleotide 7 through nucleotide 36 of SEQ ID NO: 959; (h) nucleotide 8 through nucleotide 36 of SEQ ID NO: 959; (i) nucleotide 9 through nucleotide 36 of SEQ ID NO: 959; (j) nucleotide 10 through nucleotide 36 of SEQ ID NO: 959; (k) nucleotide 11 through nucleotide 36 of SEQ ID NO: 959; (l) nucleotide 12 through nucleotide 36 of SEQ ID NO: 959; (m) nucleotide 13 through nucleotide 36 of SEQ ID NO: 959; (n) nucleotide 14 through nucleotide 36 of SEQ ID NO: 959; or (o) SEQ ID NO: 960 or SEQ ID NO: 961 or a portion thereof.

In some examples, the direct repeat sequence comprises: (a) nucleotide 1 through nucleotide 36 of a sequence that is at least 90% identical to a sequence of SEQ ID NO: 962 or SEQ ID NO: 963; (b) nucleotide 2 through nucleotide 36 of a sequence that is at least 90% identical to a sequence of SEQ ID NO: 962 or SEQ ID NO: 963; (c) nucleotide 3 through nucleotide 36 of a sequence that is at least 90% identical to a sequence of SEQ ID NO: 962 or SEQ ID NO: 963; (d) nucleotide 4 through nucleotide 36 of a sequence that is at least 90% identical to a sequence of SEQ ID NO: 962 or SEQ ID NO: 963; (e) nucleotide 5 through nucleotide 36 of a sequence that is at least 90% identical to a sequence of SEQ ID NO: 962 or SEQ ID NO: 963; (f) nucleotide 6 through nucleotide 36 of a sequence that is at least 90% identical to a sequence of SEQ ID NO: 962 or SEQ ID NO: 963; (g) nucleotide 7 through nucleotide 36 of a sequence that is at least 90% identical to a sequence of SEQ ID NO: 962 or SEQ ID NO: 963; (h) nucleotide 8 through nucleotide 36 of a sequence that is at least 90% identical to a sequence of SEQ ID NO: 962 or SEQ ID NO: 963; (i) nucleotide 9 through nucleotide 36 of a sequence that is at least 90% identical to a sequence of SEQ ID NO: 962 or SEQ ID NO: 963; (j) nucleotide 10 through nucleotide 36 of a sequence that is at least 90% identical to a sequence of SEQ ID NO: 962 or SEQ ID NO: 963; (k) nucleotide 11 through nucleotide 36 of a sequence that is at least 90% identical to a sequence of SEQ ID NO: 962 or SEQ ID NO: 963; (l) nucleotide 12 through nucleotide 36 of a sequence that is at least 90% identical to a sequence of SEQ ID NO: 962 or SEQ ID NO: 963; (m) nucleotide 13 through nucleotide 36 of a sequence that is at least 90% identical to a sequence of SEQ ID NO: 962 or SEQ ID NO: 963; (n) nucleotide 14 through nucleotide 36 of a sequence that is at least 90% identical to a sequence of SEQ ID NO: 962 or SEQ ID NO: 963; (o) nucleotide 15 through nucleotide 36 of a sequence that is at least 90% identical to a sequence of SEQ ID NO: 962 or SEQ ID NO: 963; or (p) a sequence that is at least 90% identical to a sequence of SEQ ID NO: 964 or a portion thereof.

In some examples, the direct repeat sequence comprises: (a) nucleotide 1 through nucleotide 36 of SEQ ID NO: 962 or SEQ ID NO: 963; (b) nucleotide 2 through nucleotide 36 of SEQ ID NO: 962 or SEQ ID NO: 963; (c) nucleotide 3 through nucleotide 36 of SEQ ID NO: 962 or SEQ ID NO: 963; (d) nucleotide 4 through nucleotide 36 of SEQ ID NO: 962 or SEQ ID NO: 963; (c) nucleotide 5 through nucleotide 36 of SEQ ID NO: 962 or SEQ ID NO: 963; (f) nucleotide 6 through nucleotide 36 of SEQ ID NO: 962 or SEQ ID NO: 963; (g) nucleotide 7 through nucleotide 36 of SEQ ID NO: 962 or SEQ ID NO: 963; (h) nucleotide 8 through nucleotide 36 of SEQ ID NO: 962 or SEQ ID NO: 963; (i) nucleotide 9 through nucleotide 36 of SEQ ID NO: 962 or SEQ ID NO: 963; (j) nucleotide 10 through nucleotide 36 of SEQ ID NO: 962 or SEQ ID NO: 963; (k) nucleotide 11 through nucleotide 36 of SEQ ID NO: 962 or SEQ ID NO: 963; (l) nucleotide 12 through nucleotide 36 of SEQ ID NO: 962 or SEQ ID NO: 963; (m) nucleotide 13 through nucleotide 36 of SEQ ID NO: 962 or SEQ ID NO: 963; (n) nucleotide 14 through nucleotide 36 of SEQ ID NO: 962 or SEQ ID NO: 963; (o) nucleotide 15 through nucleotide 36 of SEQ ID NO: 962 or SEQ ID NO: 963; or (p) SEQ ID NO: 964 or a portion thereof.

In some examples, the RNA guide has a sequence that is at least 90% identical to a sequence of any one of SEQ ID NOs: 989, 968, or 967. In some specific examples, the RNA guide has the sequence of any one of SEQ ID NOs: 989, 968, or 967.

In some examples, each of the first three nucleotides of the RNA guide comprises a 2′-O-methyl phosphorothioate modification.

In some examples, each of the last four nucleotides of the RNA guide comprises a 2′-O-methyl phosphorothioate modification.

In some examples, each of the first to last, second to last, and third to last nucleotides of the RNA guide comprises a 2′-O-methyl phosphorothioate modification, and wherein the last nucleotide of the RNA guide is unmodified.

In some examples, the RNA guide has a sequence that is at least 90% identical to a sequence of any one of SEQ ID NOs: 1082-1087. In some specific examples, the RNA guide has a sequence of any one of SEQ ID NOs: 1082-1087.

In some embodiments, an HAO1-targeting RNA guide comprises at least 90% identity to any one of SEQ ID NOs: 1082-1087. In some embodiments, an HAO1-targeting RNA guide comprises any one of SEQ ID NOs: 1082-1087. In some embodiments, an HAO1-targeting RNA guide comprising at least 90% identity to SEQ ID NO: 1083 or SEQ ID NO: 1084 binds the complementary region of HAO1 target sequence of SEQ ID NO: 1047 via base-pairing. In some embodiments, the HAO1-targeting RNA guide of SEQ ID NO: 1083 or SEQ ID NO: 1084 binds the complementary region of HAO1 target sequence of SEQ ID NO: 1047 via base-pairing. In some embodiments, an HAO1-targeting RNA guide comprising at least 90% identity to SEQ ID NO: 1085 or SEQ ID NO: 1086 binds the complementary region of HAO1 target sequence of SEQ ID NO: 1026 via base-pairing. In some embodiments, the HAO1-targeting RNA guide of SEQ ID NO: 1085 or SEQ ID NO: 1086 binds the complementary region of HAO1 target sequence of SEQ ID NO: 1026 via base-pairing. In some embodiments, an HAO1-targeting RNA guide comprising at least 90% identity to SEQ ID NO: 1087 or SEQ ID NO: 2293 binds the complementary region of HAO1 target sequence of SEQ ID NO: 1025 via base-pairing. In some embodiments, the HAO1-targeting RNA guide of SEQ ID NO: 1087 or SEQ ID NO: 2293 binds the complementary region of HAO1 target sequence of SEQ ID NO: 1025 via base-pairing. Embodiment 14: A nucleic acid encoding the RNA guide of Embodiment 13 as described herein.

Embodiment 15: A vector comprising the nucleic acid of Embodiment 14 as described herein.

Embodiment 16: A vector system comprising one or more vectors encoding (i) the RNA guide of Embodiment 13 as described herein, and (ii) a Cas12i polypeptide. In some examples, the vector system comprises a first vector encoding the RNA guide and a second vector encoding the Cas12i polypeptide.

Embodiment 17: A cell comprising the RNA guide, the nucleic acid, the vector, or the vector system of Embodiments 13-16 as described herein. In some examples, the cell is a eukaryotic cell, an animal cell, a mammalian cell, a human cell, a primary cell, a cell line, a stem cell, or a T cell.

Embodiment 18: A kit comprising the RNA guide, the nucleic acid, the vector, or the vector system of Embodiments 13-16 as described herein.

Embodiment 19: A method of editing an HAO1 sequence, the method comprising contacting an HAO1 sequence with the RNA guide of Embodiment 13 as described herein. In some examples, the HAO1 sequence is in a cell.

In some examples, the RNA guide induces an indel (e.g., an insertion or deletion) in the HAO1 sequence.

Embodiment 20: A method of treating primary hyperoxaluria (PH), which optionally is PH1, PH2, or PH3, in a subject, the method comprising administering the RNA guide of Embodiment 12 as described herein to the subject.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, ed. (1986»; Immobilized Cells and Enzymes (IRL Press, (1986»; and B. Perbal, A practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

The following examples are provided to further illustrate some embodiments of the present invention but are not intended to limit the scope of the invention; it will be understood by their exemplary nature that other procedures, methodologies, or techniques known to those skilled in the art may alternatively be used.

### Example 1—Cas12i2-Mediated Editing of HAO1 Target Sites in HEK293T Cells

This Example describes the genomic editing of the HAO1 gene using Cas12i2 introduced into HEK293T cells.

Cas12i2 RNA guides (crRNAs) were designed and ordered from Integrated DNA Technologies (IDT). For initial guide screening in HEK293T cells, target sequences were designed by tiling the coding exons of HAO1 for 5′-NTTN-3′ PAM sequences, and then spacer sequences were designed for the 20-bp target sequences downstream of the PAM sequence. The HAO1-targeting RNA guide sequences are shown in Table 7. In the figures, “E #T #” can also be represented as “exon #target #.”

TABLE 7
crRNA Sequences for HAO1

| | | Target | | |
|---|---|---|---|---|
| | | strand | | |
| | | (Non- | | |
| | | PAM | | |
| Guide Name | PAM* | Strand) | crRNA Sequence | Target Sequences |
| HAO1_E1T2 | CTTC | BS | AGAAAUCCGUCUUUCAU | CAAAGTCTATATATGA |
| | | | UGACGGCAAAGUCUAUA | CTAT (SEQ ID NO: 1025) |
| | | | UAUGACUAU (SEQ ID NO: | |
| | | | 967) | |
| HAO1_E1T3 | CTTT | TS | AGAAAUCCGUCUUUCAU | GGAAGTACTGATTTAG |
| | | | UGACGGGGAAGUACUGA | CATG (SEQ ID NO: 1026) |
| | | | UUUAGCAUG (SEQ ID NO: | |
| | | | 968) | |
| HAO1_E1T4 | CTTC | TS | AGAAAUCCGUCUUUCAU | ATCATTTGCCCCAGAC |
| | | | UGACGGAUCAUUUGCCC | CTGT (SEQ ID NO: 1027) |
| | | | CAGACCUGU (SEQ ID NO: | |
| | | | 969) | |
| HAO1_E1T5 | CTTT | BS | AGAAAUCCGUCUUUCAU | GGCTGATAATATTGCA |
| | | | UGACGGGGCUGAUAAUA | GCAT (SEQ ID NO: 1028) |
| | | | UUGCAGCAU (SEQ ID NO: | |
| | | | 970) | |
| HAO1_E1T6 | ATTT | BS | AGAAAUCCGUCUUUCAU | GTATCAATGATTATGA |
| | | | UGACGGGUAUCAAUGAU | ACAA (SEQ ID NO: 1029) |
| | | | UAUGAACAA (SEQ ID NO: | |
| | | | 971) | |
| HAO1_E1T7 | TTTG | BS | AGAAAUCCGUCUUUCAU | TATCAATGATTATGAA |
| | | | UGACGGUAUCAAUGAUU | CAAC (SEQ ID NO: 1030) |
| | | | AUGAACAAC (SEQ ID NO: | |
| | | | 972) | |
| HAO1_E1T9 | ATTA | BS | AGAAAUCCGUCUUUCAU | TGAACAACATGCTAAA |
| | | | UGACGGUGAACAACAUG | TCAG (SEQ ID NO: 1031) |
| | | | CUAAAUCAG (SEQ ID NO: | |
| | | | 973) | |
| HAO1_E1T11 | TTTA | TS | AGAAAUCCGUCUUUCAU | GCATGTTGTTCATAAT |
| | | | UGACGGGCAUGUUGUUC | CATT (SEQ ID NO: 1032) |
| | | | AUAAUCAUU (SEQ ID NO: | |
| | | | 974) | |
| HAO1_E1T12 | ATTT | TS | AGAAAUCCGUCUUUCAU | AGCATGTTGTTCATAA |
| | | | UGACGGAGCAUGUUGUU | TCAT (SEQ ID NO: 1033) |
| | | | CAUAAUCAU (SEQ ID NO: | |
| | | | 975) | |
| HAO1_E1T13 | TTTG | TS | AGAAAUCCGUCUUUCAU | GAAGTACTGATTTAGC |
| | | | UGACGGGAAGUACUGAU | ATGT (SEQ ID NO: 1034) |
| | | | UUAGCAUGU (SEQ ID NO: | |
| | | | 976) | |
| HAO1_E1T14 | ATTA | BS | AGAAAUCCGUCUUUCAU | CAGGTCTGGGGCAAAT |
| | | | UGACGGCAGGUCUGGGG | GATG (SEQ ID NO: 1035) |
| | | | CAAAUGAUG (SEQ ID NO: | |
| | | | 977) | |
| HAO1_E1T15 | TTTG | TS | AGAAAUCCGUCUUUCAU | CCCCAGACCTGTAATA |
| | | | UGACGGCCCCAGACCUG | GTCA (SEQ ID NO: 1036) |
| | | | UAAUAGUCA (SEQ ID NO: | |
| | | | 978) | |
| HAO1_E1T16 | ATTT | TS | AGAAAUCCGUCUUUCAU | GCCCCAGACCTGTAAT |
| | | | UGACGGGCCCCAGACCU | AGTC (SEQ ID NO: 1037) |
| | | | GUAAUAGUC (SEQ ID NO: | |
| | | | 979) | |
| HAO1_E1T17 | TTTC | TS | AGAAAUCCGUCUUUCAU | TTCATCATTTGCCCCAG |
| | | | UGACGGUUCAUCAUUUG | ACC (SEQ ID NO: 1038) |
| | | | CCCCAGACC (SEQ ID NO: | |
| | | | 980) | |
| HAO1_E1T18 | GTTT | TS | AGAAAUCCGUCUUUCAU | CTTCATCATTTGCCCCA |
| | | | UGACGGCUUCAUCAUUU | GAC (SEQ ID NO: 1039) |
| | | | GCCCCAGAC (SEQ ID NO: | |
| | | | 981) | |
| HAO1_E1T19 | TTTG | BS | AGAAAUCCGUCUUUCAU | AATGCTGCAATATTAT |
| | | | UGACGGAAUGCUGCAAU | CAGC (SEQ ID NO: 1040) |
| | | | AUUAUCAGC (SEQ ID NO: | |
| | | | 982) | |
| HAO1_E1T23 | TTTT | TS | AGAAAUCCGUCUUUCAU | CTTACCTGGAAAATGC |
| | | | UGACGGCUUACCUGGAA | TGCA (SEQ ID NO: 1041) |
| | | | AAUGCUGCA (SEQ ID NO: | |
| | | | 983) | |
| HAO1_E1T24 | ATTT | TS | AGAAAUCCGUCUUUCAU | TCTTACCTGGAAAATG |
| | | | UGACGGUCUUACCUGGA | CTGC (SEQ ID NO: 1042) |
| | | | AAAUGCUGC (SEQ ID NO: | |
| | | | 984) | |
| HAO1_E2T1 | CTTC | BS | AGAAAUCCGUCUUUCAU | TGTTTTAGGACAGAGG |
| | | | UGACGGUGUUUUAGGAC | GTCA (SEQ ID NO: 1043) |
| | | | AGAGGGUCA (SEQ ID NO: | |
| | | | 985) | |
| HAO1_E2T2 | CTTC | TS | AGAAAUCCGUCUUUCAU | CTCCTACCTCTCACAGT |
| | | | UGACGGCUCCUACCUCU | GGC (SEQ ID NO: 1044) |
| | | | CACAGUGGC (SEQ ID NO: | |
| | | | 986) | |
| HAO1_E2T3 | TTTA | BS | AGAAAUCCGUCUUUCAU | ATTCTAGATGGAAGCT |
| | | | UGACGGAUUCUAGAUGG | GTAT (SEQ ID NO: 1045) |
| | | | AAGCUGUAU (SEQ ID NO: | |
| | | | 987) | |
| HAO1_E2T4 | ATTC | BS | AGAAAUCCGUCUUUCAU | TAGATGGAAGCTGTAT |
| | | | UGACGGUAGAUGGAAGC | CCAA (SEQ ID NO: 1046) |
| | | | UGUAUCCAA (SEQ ID NO: | |
| | | | 988) | |
| HAO1_E2T5 | ATTC | TS | AGAAAUCCGUCUUUCAU | CGGAGCATCCTTGGAT |
| | | | UGACGGCGGAGCAUCCU | ACAG (SEQ ID NO: 1047) |
| | | | UGGAUACAG (SEQ ID NO: | |
| | | | 989) | |
| HAO1_E2T6 | GTTG | BS | AGAAAUCCGUCUUUCAU | CTGAAACAGATCTGTC |
| | | | UGACGGCUGAAACAGAU | GACT (SEQ ID NO: 1048) |
| | | | CUGUCGACU (SEQ ID NO: | |
| | | | 990) | |
| HAO1_E2T7 | TTTC | TS | AGAAAUCCGUCUUUCAU | AGCAACATTCCGGAGC |
| | | | UGACGGAGCAACAUUCC | ATCC (SEQ ID NO: 1049) |
| | | | GGAGCAUCC (SEQ ID NO: | |
| | | | 991) | |
| HAO1_E2T8 | GTTT | TS | AGAAAUCCGUCUUUCAU | CAGCAACATTCCGGAG |
| | | | UGACGGCAGCAACAUUC | CATC (SEQ ID NO: 1050) |
| | | | CGGAGCAUC (SEQ ID NO: | |
| | | | 992) | |
| HAO1_E2T9 | GTTT | BS | AGAAAUCCGUCUUUCAU | TAGGACAGAGGGTCAG |
| | | | UGACGGUAGGACAGAGG | CATG (SEQ ID NO: 1051) |
| | | | GUCAGCAUG (SEQ ID NO: | |
| | | | 993) | |
| HAO1_E2T10 | TTTT | BS | AGAAAUCCGUCUUUCAU | AGGACAGAGGGTCAGC |
| | | | UGACGGAGGACAGAGGG | ATGC (SEQ ID NO: 1052) |
| | | | UCAGCAUGC (SEQ ID NO: | |
| | | | 994) | |
| HAO1_E2T11 | TTTA | BS | AGAAAUCCGUCUUUCAU | GGACAGAGGGTCAGCA |
| | | | UGACGGGGACAGAGGGU | TGCC (SEQ ID NO: 1053) |
| | | | CAGCAUGCC (SEQ ID NO: | |
| | | | 995) | |
| HAO1_E3T1 | CTTT | BS | AGAAAUCCGUCUUUCAU | CTTTCTCAGCCTGTCAG |
| | | | UGACGGCUUUCUCAGCC | TCC (SEQ ID NO: 1054) |
| | | | UGUCAGUCC (SEQ ID NO: | |
| | | | 996) | |
| HAO1_E3T2 | CTTT | BS | AGAAAUCCGUCUUUCAU | CTCAGCCTGTCAGTCC |
| | | | UGACGGCUCAGCCUGUC | CTGG (SEQ ID NO: 1055) |
| | | | AGUCCCUGG (SEQ ID NO: | |
| | | | 997) | |
| HAO1_E3T3 | GTTC | TS | AGAAAUCCGUCUUUCAU | CCAGGGACTGACAGGC |
| | | | UGACGGCCAGGGACUGA | TGAG (SEQ ID NO: 1056) |
| | | | CAGGCUGAG (SEQ ID NO: | |
| | | | 998) | |
| HAO1_E3T4 | GTTC | BS | AGAAAUCCGUCUUUCAU | CTGGGCCACCTCCTCA |
| | | | UGACGGCUGGGCCACCU | ATTG (SEQ ID NO: 1057) |
| | | | CCUCAAUUG (SEQ ID NO: | |
| | | | 999) | |
| HAO1_E3T5 | CTTC | TS | AGAAAUCCGUCUUUCAU | AATTGAGGAGGTGGCC |
| | | | UGACGGAAUUGAGGAGG | CAGG (SEQ ID NO: 1058) |
| | | | UGGCCCAGG (SEQ ID NO: | |
| | | | 1000) | |
| HAO1_E3T6 | CTTC | TS | AGAAAUCCGUCUUUCAU | TTCAATTGAGGAGGTG |
| | | | UGACGGUUCAAUUGAGG | GCCC (SEQ ID NO: 1059) |
| | | | AGGUGGCCC (SEQ ID NO: | |
| | | | 1001) | |
| HAO1_E3T7 | CTTC | TS | AGAAAUCCGUCUUUCAU | CGCCACTTCTTCAATTG |
| | | | UGACGGCGCCACUUCUU | AGG (SEQ ID NO: 1060) |
| | | | CAAUUGAGG (SEQ ID NO: | |
| | | | 1002) | |
| HAO1_E3T8 | CTTC | BS | AGAAAUCCGUCUUUCAU | GTTGGCTGCAACTGTA |
| | | | UGACGGGUUGGCUGCAA | TATC (SEQ ID NO: 1061) |
| | | | CUGUAUAUC (SEQ ID NO: | |
| | | | 1003) | |
| HAO1_E3T9 | CTTC | TS | AGAAAUCCGUCUUUCAU | TCGGTCCTTGTAGATA |
| | | | UGACGGUCGGUCCUUGU | TACA (SEQ ID NO: 1062) |
| | | | AGAUAUACA (SEQ ID NO: | |
| | | | 1004) | |
| HAO1_E3T11 | CTTC | TS | AGAAAUCCGUCUUUCAU | TCTGCCTGCCGCACTA |
| | | | UGACGGUCUGCCUGCCG | GCTT (SEQ ID NO: 1063) |
| | | | CACUAGCUU (SEQ ID NO: | |
| | | | 1005) | |
| HAO1_E3T12 | TTTC | BS | AGAAAUCCGUCUUUCAU | TTTCTCAGCCTGTCAGT |
| | | | UGACGGUUUCUCAGCCU | CCC (SEQ ID NO: 1064) |
| | | | GUCAGUCCC (SEQ ID NO: | |
| | | | 1006) | |
| HAO1_E3T13 | TTTC | BS | AGAAAUCCGUCUUUCAU | TCAGCCTGTCAGTCCC |
| | | | UGACGGUCAGCCUGUCA | TGGG (SEQ ID NO: 1065) |
| | | | GUCCCUGGG (SEQ ID NO: | |
| | | | 1007) | |
| HAO1_E3T14 | GTTG | BS | AGAAAUCCGUCUUUCAU | AGTTCCTGGGCCACCT |
| | | | UGACGGAGUUCCUGGGC | CCTC (SEQ ID NO: 1066) |
| | | | CACCUCCUC (SEQ ID NO: | |
| | | | 1008) | |
| HAO1_E3T15 | ATTG | TS | AGAAAUCCGUCUUUCAU | AGGAGGTGGCCCAGGA |
| | | | UGACGGAGGAGGUGGCC | ACTC (SEQ ID NO: 1067) |
| | | | CAGGAACUC (SEQ ID NO: | |
| | | | 1009) | |
| HAO1_E3T16 | ATTG | BS | AGAAAUCCGUCUUUCAU | AAGAAGTGGCGGAAG |
| | | | UGACGGAAGAAGUGGCG | CTGGT (SEQ ID NO: |
| | | | GAAGCUGGU (SEQ ID NO: | 1068) |
| | | | 1010) | |
| HAO1_E3T17 | GTTG | BS | AGAAAUCCGUCUUUCAU | GCTGCAACTGTATATC |
| | | | UGACGGGCUGCAACUGU | TACA (SEQ ID NO: 1069) |
| | | | AUAUCUACA (SEQ ID NO: | |
| | | | 1011) | |
| HAO1_E3T18 | GTTG | TS | AGAAAUCCGUCUUUCAU | CAGCCAACGAAGTGCC |
| | | | UGACGGCAGCCAACGAA | TCAG (SEQ ID NO: 1070) |
| | | | GUGCCUCAG (SEQ ID NO: | |
| | | | 1012) | |
| HAO1_E3T19 | CTTG | TS | AGAAAUCCGUCUUUCAU | TAGATATACAGTTGCA |
| | | | UGACGGUAGAUAUACAG | GCCA (SEQ ID NO: 1071) |
| | | | UUGCAGCCA (SEQ ID NO: | |
| | | | 1013) | |
| HAO1_E3T20 | CTTG | TS | AGAAAUCCGUCUUUCAU | GTGACTTCTCGGTCCTT |
| | | | UGACGGGUGACUUCUCG | GTA (SEQ ID NO: 1072) |
| | | | GUCCUUGUA (SEQ ID NO: | |
| | | | 1014) | |
| HAO1_E3T22 | ATTT | BS | AGAAAUCCGUCUUUCAU | GTGACAGTGGACACAC |
| | | | UGACGGGUGACAGUGGA | CTTA (SEQ ID NO: 1073) |
| | | | CACACCUUA (SEQ ID NO: | |
| | | | 1015) | |
| HAO1_E3T23 | TTTG | BS | AGAAAUCCGUCUUUCAU | TGACAGTGGACACACC |
| | | | UGACGGUGACAGUGGAC | TTAC (SEQ ID NO: 1074) |
| | | | ACACCUUAC (SEQ ID NO: | |
| | | | 1016) | |
| HAO1_E3T24 | CTTA | BS | AGAAAUCCGUCUUUCAU | CCTGGGCAACCGTCTG |
| | | | UGACGGCCUGGGCAACC | GATG (SEQ ID NO: 1075) |
| | | | GUCUGGAUG (SEQ ID NO: | |
| | | | 1017) | |
| HAO1_E3T25 | GTTG | TS | AGAAAUCCGUCUUUCAU | CCCAGGTAAGGTGTGT |
| | | | UGACGGCCCAGGUAAGG | CCAC (SEQ ID NO: 1076) |
| | | | UGUGUCCAC (SEQ ID NO: | |
| | | | 1018) | |
| HAO1_E3T26 | GTTA | TS | AGAAAUCCGUCUUUCAU | CGCACATCATCCAGAC |
| | | | UGACGGCGCACAUCAUC | GGTT (SEQ ID NO: 1077) |
| | | | CAGACGGUU (SEQ ID NO: | |
| | | | 1019) | |
| HAO1_E3T27 | TTTG | TS | AGAAAUCCGUCUUUCAU | AATCTGTTACGCACAT |
| | | | UGACGGAAUCUGUUACG | CATC (SEQ ID NO: 1078) |
| | | | CACAUCAUC (SEQ ID NO: | |
| | | | 1020) | |
| HAO1_E3T28 | GTTT | TS | AGAAAUCCGUCUUUCAU | GAATCTGTTACGCACA |
| | | | UGACGGGAAUCUGUUAC | TCAT (SEQ ID NO: 1079) |
| | | | GCACAUCAU (SEQ ID NO: | |
| | | | 1021) | |
| HAO1_E3T29 | GTTG | TS | AGAAAUCCGUCUUUCAU | TGGCGGCAGTTTGAAT |
| | | | UGACGGUGGCGGCAGUU | CTGT (SEQ ID NO: 1080) |
| | | | UGAAUCUGU (SEQ ID NO: | |
| | | | 1022) | |
| HAO1_E3T30 | GTTA | TS | AGAAAUCCGUCUUUCAU | CCTGAGTTGTGGCGGC |
| | | | UGACGGCCUGAGUUGUG | AGTT (SEQ ID NO: 1081) |
| | | | GCGGCAGUU (SEQ ID NO: | |
| | | | 1023) | |

*The 3′ three nucleotides represent the 5′-TTN-3′ motif.

Cas12i2 RNP complexation reactions were made by mixing purified Cas12i2 polypeptide of SEQ ID NO: 924 (400 μM) with an HAO1-targeting crRNA (1 mM in 250 mM NaCl) at a 1:1 (Cas12i2:crRNA) volume ratio (2.5:1 crRNA:Cas12i2 molar ratio). Complexations were incubated on ice for 30-60 min.

HEK293T cells were harvested using TRYPLE™ (recombinant cell-dissociation enzymes; ThermoFisher) and counted. Cells were washed once with PBS and resuspended in SF buffer+supplement (SF CELL LINE 4D-NUCLEOFECTOR™ X KIT S; Lonza #V4XC-2032) at a concentration of 16,480 cells/μL. Resuspended cells were dispensed at 3×10⁵ cells/reaction into Lonza 16-well NUCLEOCUVETTE® strips. Complexed Cas12i2 RNP was added to each reaction at a final concentration of 10 μM (Cas12i2), and transfection enhancer oligos were then added at a final concentration of 4 μM. The final volume of each electroporated reaction was 20 μL. Non-targeting guides were used as negative controls.

The strips were electroporated using an electroporation device (program CM-130, Lonza 4D-NUCLEOFECTOR™). Immediately following electroporation, 80 μL of pre-warmed DMEM+10% FBS was added to each well and mixed gently by pipetting. For each technical replicate plate, plated 10 μL (30,000 cells) of diluted nucleofected cells into pre-warmed 96-well plate with wells containing 100 μL DMEM+10% FBS. Editing plates were incubated for 3 days at 37° C. with 5% CO₂.

After 3 days, wells were harvested using TRYPLE™ (recombinant cell-dissociation enzymes; ThermoFisher) and transferred to 96-well TWIN.TEC® PCR plates (Eppendorf). Media was flicked off and cells were resuspended in 20 μL QUICKEXTRACT™ (DNA extraction buffer; Lucigen). Samples were then cycled in a PCR machine at 65° C. for 15 min, 68° C. for 15 min, 98° C. for 10 min. Samples were then frozen at −20° C.

Samples for Next Generation Sequencing (NGS) were prepared by rounds of PCR. The first round (PCR I) was used to amplify the genomic regions flanking the target site and add NGS adapters. The second round (PCR II) was used to add NGS indexes. Reactions were then pooled, purified by column purification, and quantified on a fluorometer (Qubit). Sequencing runs were done using a 150 cycle NGS instrument (NEXTSEQ™ v2.5) mid or high output kit (Illumina) and run on an NGS instrument (NEXTSEQ™ 550; Illumina).

For NGS analysis, the indel mapping function used a sample's fastq file, the amplicon reference sequence, and the forward primer sequence. For each read, a kmer-scanning algorithm was used to calculate the edit operations (match, mismatch, insertion, deletion) between the read and the reference sequence. In order to remove small amounts of primer dimer present in some samples, the first 30 nt of each read was required to match the reference and reads where over half of the mapping nucleotides are mismatches were filtered out as well. Up to 50,000 reads passing those filters were used for analysis, and reads were counted as an indel read if they contained an insertion or deletion. The % indels was calculated as the number of indel-containing reads divided by the number of reads analyzed (reads passing filters up to 50,000). The QC standard for the minimum number of reads passing filters was 10,000.

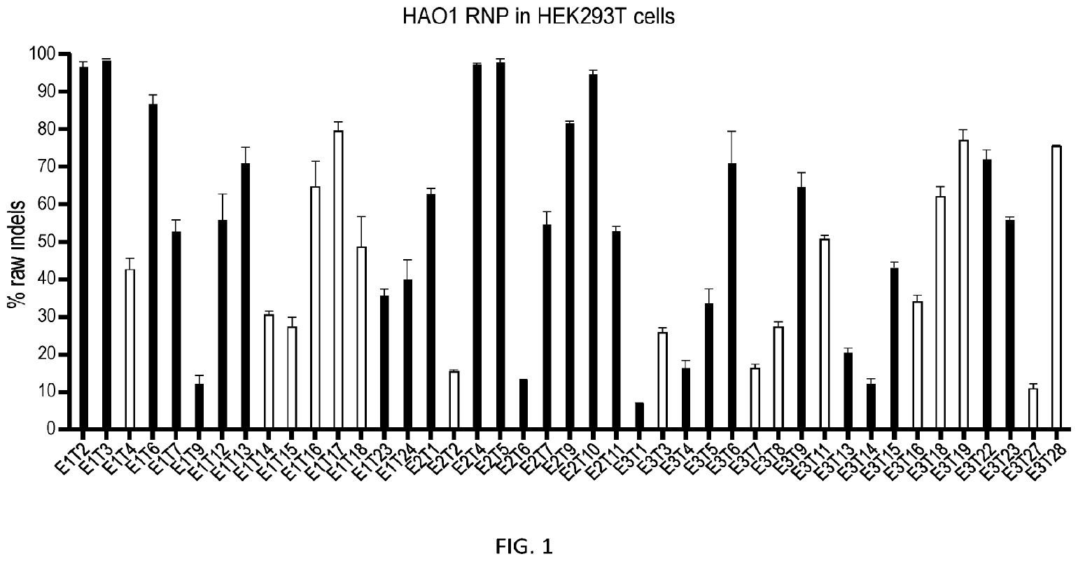

FIG. 1 shows HAO1 indels in HEK293T cells following RNP delivery. Error bars represent the average of three technical replicates across one biological replicate. Following delivery, indels were detected within and/or adjacent to each of the HAO1 target sites with each of the RNA guides. Delivery of E1T2, E1T3, E1T6, E1T7, E1T13, T1T17, E2T4, E2T5, E2T9, E2T10, E3T6, E3T19, E3T22, and E3T28 resulted in indels in over 70% of the NGS reads. Therefore, HAO1-targeting RNA guides induced indels in exon 1, exon 2, and exon 3 in HEK293T cells.

This Example thus shows that HAO1 can be individually targeted by Cas12i2 RNPs in mammalian cells such as HEK293T cells.

### Example 2—Cas12i2-Mediated Editing of HAO1 Target Sites in HepG2 Cells

This Example describes the genomic editing of the HAO1 gene using Cas12i2 introduced into HepG2 cells by RNP.

RNP complexation reactions were performed as described in Example 1 with various RNA guides of Table 7. HepG2 cells were harvested using TRYPLE™ (recombinant cell-dissociation enzymes; ThermoFisher) and counted. Cells were washed once with PBS and resuspended in SF buffer+supplement (SF CELL LINE 4D-NUCLEOFECTOR™ X KIT S; Lonza #V4XC-2032) at a concentration of 13,889 cells/μL. Resuspended cells were dispensed at 2.5e5 cells/reaction into Lonza 16-well NUCLEOCUVETTE® strips. Complexed Cas12i2 RNP was added to each reaction at a final concentration of 20 μM (Cas12i2), with no transfection enhancer oligo. The final volume of each electroporated reaction was 20 μL. Non-targeting guides were used as negative controls.

The strips were electroporated using an electroporation device (program DJ-100, Lonza 4D-NUCLEOFECTOR™). Immediately following electroporation, 80 μL of pre-warmed EMEM+10% FBS was added to each well and mixed gently by pipetting. For each technical replicate plate, plated 10 μL (25,000 cells) of diluted nucleofected cells into pre-warmed 96-well plate with wells containing 100 μL EMEM+10% FBS. Editing plates were incubated for 3 days at 37° C. with 5% CO₂.

After 3 days, wells were harvested using TRYPLE™ (recombinant cell-dissociation enzymes; ThermoFisher) and transferred to 96-well TWIN.TEC® PCR plates (Eppendorf). Media was flicked off and cells were resuspended in 20 μL QUICKEXTRACT™ (DNA extraction buffer; Lucigen). Samples were then cycled in a PCR machine at 65° C. for 15 min, 68° C. for 15 min, 98° C. for 10 min. Samples were then frozen at −20° C. Samples were analyzed by NGS as described in Example 1.

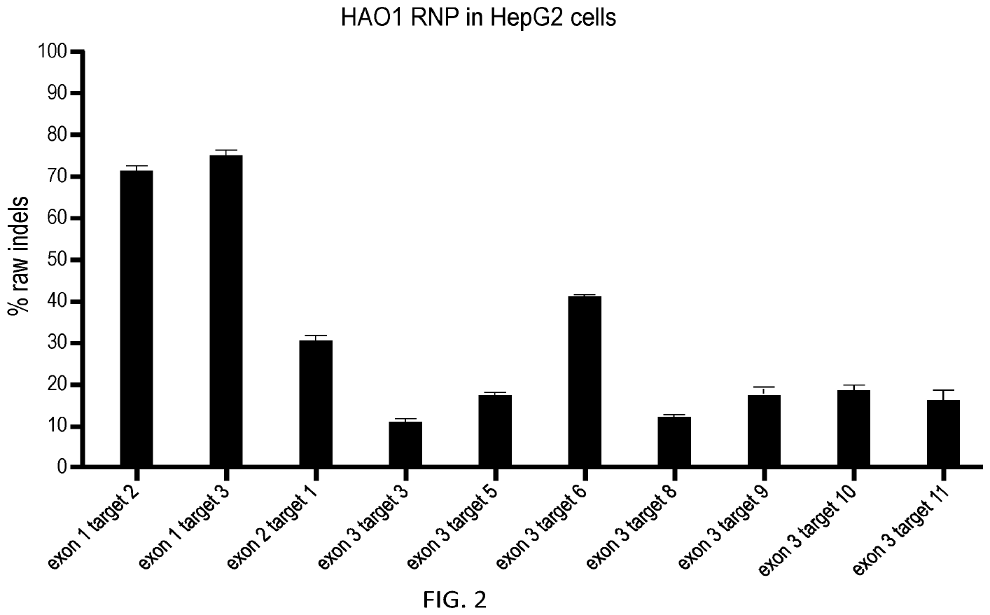

FIG. 2 shows HAO1 indels in HepG2 cells following RNP delivery. Error bars represent the average of three technical replicates across one biological replicate. Following delivery, indels were detected within and/or adjacent to each of the HAO1 target sites with each of the RNA guides. Therefore, HAO1-targeting RNA guides induced indels in exon 1, exon 2, and exon 3 in HepG2 cells.

This Example thus shows that HAO1 can be targeted by Cas12i2 RNPs in mammalian cells such as HepG2 cells.

### Example 3—Cas12i2-Mediated Editing of HAO1 Target Sites in Primary Hepatocytes

This Example describes the genomic editing of the HAO1 using Cas12i2 introduced into primary hepatocytes cells by RNP.

RNP complexation reactions were performed as described in Example 1 with RNA guides of Table 7. Primary hepatocyte cells from human donors were thawed from liquid nitrogen very quickly in a 37° C. water bath. The cells were added to pre-warmed hepatocyte recovery media (Thermofisher, CM7000) and centrifuged at 100 g for 10 minutes. The cell pellet was resuspended in appropriate volume of hepatocyte plating Medium (Williams' Medium E, Thermofisher A1217601 supplemented with Hepatocyte Plating Supplement Pack (serum-containing), Thermofisher CM3000). The cells were subjected to trypan blue viability count with an INCUCYTE® disposable hemocytometer (Fisher scientific, 22-600-100). The cells were then washed in PBS and resuspended in P3 buffer+supplement (P3 PRIMARY CELL 4D-NUCLEOFECTOR™ X Kit; Lonza, VXP-3032) at a concentration of ˜7,500 cells/μL. Resuspended cells were dispensed at 150,000 cells/reaction into the 16 well Lonza NUCLEOCUVETTE strips or 500,000 cells/reaction into the single Lonza NUCLEOCUVETTES® for the mRNA readout. Complexed Cas12i2 RNP was added to each reaction at a final concentration of 20 μM (Cas12i2), and transfection enhancer oligos were then added at a final concentration of 4 μM. The final volume of each electroporated reaction was either 20 μL in the 16 well nucleocuvette strip format or 100 μL in the single nucleocuvette format. Non-targeting guides were used as negative controls.

The strips were electroporated using DS-150 program, while the single nucleocuvettes were electroporated using CA137 program (Lonza 4D-NUCLEOFECTOR™). Immediately following electroporation, pre-warmed Hepatocyte plating medium was added to each well and mixed very gently by pipetting. For each technical replicate plate, plated all the cell suspension of diluted nucleofected cells into a pre-warmed collagen-coated 96-well plate or 24-well plate (Thermofisher) with wells containing Hepatocyte plating medium. The cells were then incubated in a 37° C. incubator. The media was changed to hepatocyte maintenance media (Williams' Medium E, Thermofisher A1217601 supplemented with William's E medium Cell Maintenance Cocktail, Thermofisher CM 4000) after the cells attached after 4 hours. Fresh hepatocyte maintenance media was replaced after 2 days.

After 4-5 days post RNP electroporation, media was aspirated and the cells were harvested by shaking (500 rpm) in a 37° C. incubator with 2 mg/ml collagenase IV (Thermofisher, 17104019) dissolved in PBS containing Ca/Mg (Thermofisher). After cells were dissociated from the plate, they were transferred to 96-well TWIN.TEC® PCR plates (Eppendorf) and centrifuged. Media was flicked off and cell pellets for the NGS readout were resuspended in 20 μL QUICKEXTRACT™ (DNA extraction buffer; Lucigen). Samples were then cycled in a PCR machine at 65° C. for 15 min, 68° C. for 15 min, 98° C. for 10 min and analyzed by NGS as described in Example 1.

For the mRNA readout, cell pellets were frozen at −80° C. and subsequently resuspended in lysis buffer and DNA/RNA extracted with the RNeasy kit (Qiagen) following manufacturer's instructions. The DNA extracted from the samples were analyzed by NGS. The RNA isolated was checked for quantity and purity using nanodrop, and subsequently used for cDNA synthesis using 5× iScript reverse transcription reaction mix (Bio-Rad laboratories), following manufacturer's recommendations. cDNA templated was appropriately diluted to be in linear range of the subsequent analysis. Diluted cDNA was used to set up a 20 μL Digital Droplet PCR (ddPCR-BioRad laboratories) reaction using target-specific primer and probe for HAO1. ATTGTGCACTGTCAGATCTTGGAAACGGCCAAAGGATTTTTCCTCACCAATGTCTTG TCGATGACTTTCACATTCTGGCACCCACTCAGAGCCATGGCCAACCGGAATTCTTCC TTTAGTAT (SEQ ID NO: 1088), and 2× ddPCR Supermix for Probes No dUTP (BioRad laboratories) following manufacturer's instructions. The reaction was used to generate droplets using Automated Droplet Generator (BioRad Laboratories), following manufacture's recommendations. The plate was sealed using PX1 PCR Plate Sealer (BioRad Laboratories) generated droplets were subjected to PCR amplification using C1000 Touch Thermal Cycler (BioRad Laboratories) using conditions recommended by the manufacturer. The PCR amplified droplets were read on QX200 Droplet Reader (BioRad Laboratories) and the acquired data was analyzed using QX Manager version 1.2 (BioRad Laboratories) to determine presence of absolute copy number of mRNA present in each reaction for the appropriate targets.

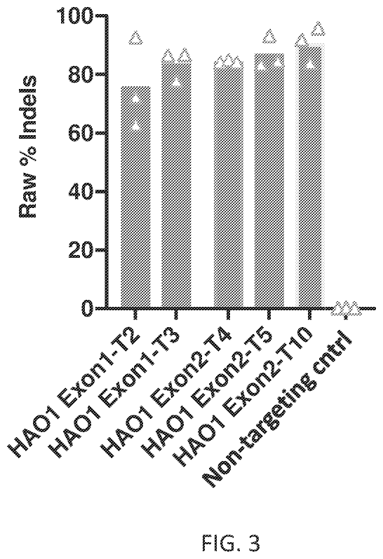

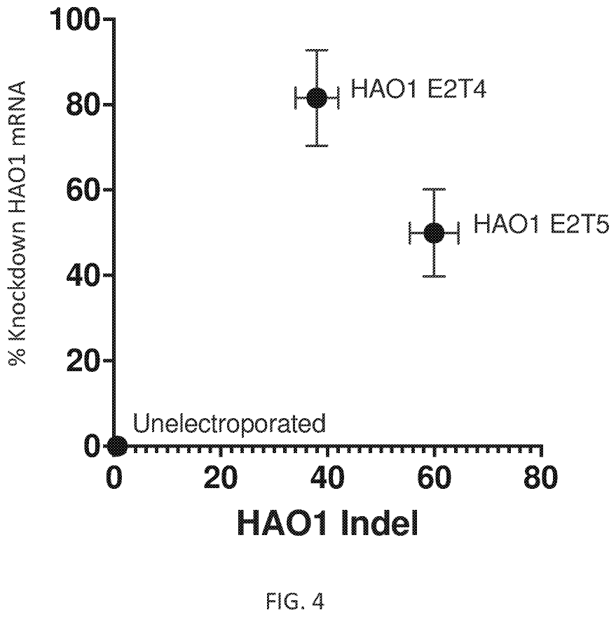

As shown in FIG. 3, each RNA guide tested induced indels within and/or adjacent to the HAO1 target sites. Indels were not induced with the non-targeting control. Therefore, HAO1-targeting RNA guides induced indels in primary hepatocytes. Indels were then correlated with mRNA levels for each target to determine whether indels lead to mRNA knockdown and subsequent protein knockdown. FIG. 4 shows % mRNA knockdown of HAO1 in edited cells compared to unedited control cells. Although a higher percentage of NGS reads comprised indels using HAO1 E2T5 (SEQ ID NO: 989) compared to HAO1 E2T4 (SEQ ID NO: 988), HAO1 E2T4 resulted in a greater knockdown of HAO1 mRNA.

This Example thus shows that HAO1 can be targeted by Cas12i2 RNPs in mammalian cells such as primary human hepatocytes.

### Example 4—Editing of HAO1 Target Sites in HepG2 Cells with Cas12i2 Variants

This Example describes indel assessment on HAO1 targets using variants introduced into HepG2 cells by transient transfection.

The Cas12i2 variants of SEQ ID NO: 924 and SEQ ID NO: 927 were individually cloned into a pcda3.1 backbone (Invitrogen). Nucleic acids encoding RNA guides E1T2 (SEQ ID NO: 967), E1T3 (SEQ ID NO: 968), E2T4 (SEQ ID NO: 988), E2T5 (SEQ ID NO: 989), E2T10 (SEQ ID NO: 994) were cloned into a pUC19 backbone (New England Biolabs). The plasmids were then maxi-prepped and diluted.

HepG2 cells were harvested using TRYPLE™ (recombinant cell-dissociation enzymes; ThermoFisher) and counted. Cells were washed once with PBS and resuspended in SF buffer+supplement (SF CELL LINE 4D-NUCLEOFECTOR™ X KIT S; Lonza #V4XC-2032).

Approximately 16 hours prior to transfection, 25,000 HepG2 cells in EMEM/10% FBS were plated into each well of a 96-well plate. On the day of transfection, the cells were 70-90% confluent. For each well to be transfected, a mixture of Lipofectamine™ 3000 and Opti-MEM® was prepared and then incubated at room temperature for 5 minutes (Solution 1). After incubation, the Lipofectamine™: OptiMEM® mixture was added to a separate mixture containing nuclease plasmid and RNA guide plasmid and P3000 reagent (Solution 2). In the case of negative controls, the crRNA was not included in Solution 2. The Solution 1 and Solution 2 were mixed by pipetting up and down and then incubated at room temperature for 15 minutes. Following incubation, the Solution 1 and Solution 2 mixture was added dropwise to each well of a 96 well plate containing the cells.

After 3 days, wells were harvested using TRYPLE™ (recombinant cell-dissociation enzymes; ThermoFisher) and transferred to 96-well TWIN.TEC® PCR plates (Eppendorf). Media was flicked off and cells were resuspended in 20 μL QUICKEXTRACT™ (DNA extraction buffer; Lucigen). Samples were then cycled in a PCR machine at 65° C. for 15 min, 68° C. for 15 min, 98° C. for 10 min. Samples were then frozen at −20° C. and analyzed by NGS as described in Example 1.

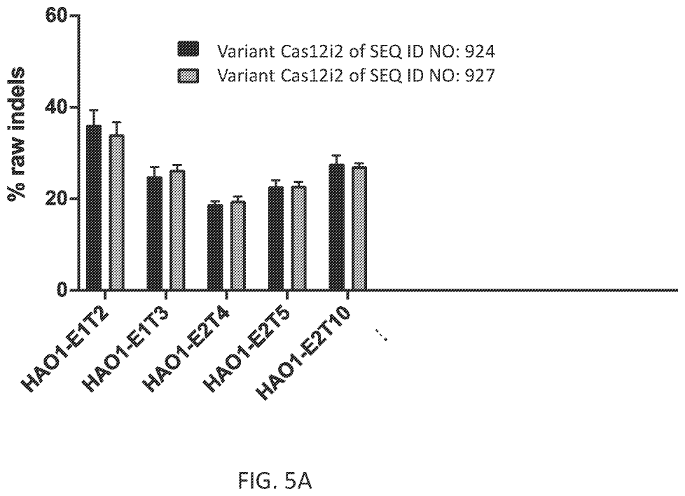

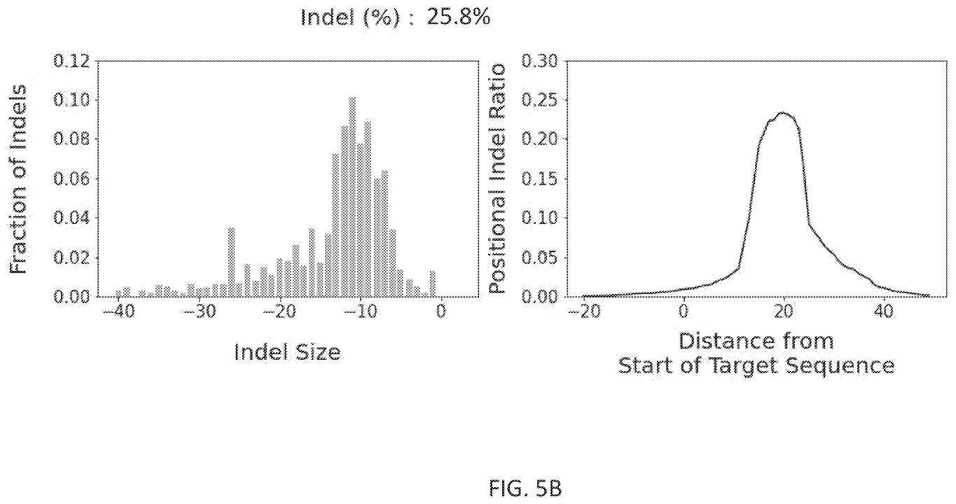

As shown in FIG. 5A, comparable indel activity with the two Cas12i2 variants was observed for E1T2, E1T3, E2T4, E2T5, E2T10. FIG. 5B shows the indel size frequency (left) and indel start position relative to the PAM for E1T3 and the variant Cas12i2 of SEQ ID NO: 924. As shown on the left, deletions ranged in size from 1 nucleotide to about 40 nucleotides. The majority of the deletions were about 6 nucleotides to about 27 nucleotides in length. As shown on the right, the target sequence is represented as starting at position 0 and ending at position 20. Indels started within about 10 nucleotides and about 35 nucleotides downstream of the PAM sequence. The majority of indels started near the end of the target sequence, e.g., about 18 nucleotides to about 25 nucleotides downstream of the PAM sequence.

Thus, this Example shows that HAO1 is capable of being targeted by multiple Cas12i2 polypeptides.

### Example 5—Editing of HAO1 in Primary Human Hepatocytes Using Cas12i2 mRNA Constructs

This Example describes indel assessment on HAO1 target sites via delivery of Cas12i2 mRNA and chemically modified HAO1-targeting RNA guides.

mRNA sequences corresponding to the variant Cas12i2 sequence of SEQ ID NO: 924 and the variant Cas12i2 sequence of SEQ ID NO: 927 were synthesized by Aldeveron with 1-pseudo-U modified nucleotides and using CleanCap® Reagent AG (TriLink Biotechnologies). The Cas12i2 mRNA sequences, shown in Table 8, further comprised a C-terminal NLS.

TABLE 8
Cas1212 mRNA Sequences

| Description | mRNA Sequence |
|---|---|
| mRNA | AUGAGCUCCGCCAUCAAGUCCUACAAGUCUGUGCUGCGGCCAAACGAGAGAAAGAAUCAGC |
| corresponding to | UGCUGAAGUCCACCAUCCAGUGCCUGGAGGACGGCUCCGCCUUCUUUUUCAAGAUGCUGCA |
| variant Cas1212 | GGGCCUGUUUGGCGGCAUCACCCCCGAGAUCGUGAGAUUCAGCACAGAGCAGGAGAAGCAG |
| of SEQ ID NO: | CAGCAGGAUAUCGCCCUGUGGUGUGCCGUGAAUUGGUUCAGGCCUGUGAGCCAGGACUCCC |
| 924 | UGACCCACACAAUCGCCUCCGAUAACCUGGUGGAGAAGUUUGAGGAGUACUAUGGCGGCAC |
| | AGCCAGCGACGCCAUCAAGCAGUACUUCAGCGCCUCCAUCGGCGAGUCCUACUAUUGGAAU |
| | GACUGCCGCCAGCAGUACUAUGAUCUGUGUCGGGAGCUGGGCGUGGAGGUGUCUGACCUGA |
| | CCCACGAUCUGGAGAUCCUGUGCCGGGAGAAGUGUCUGGCCGUGGCCACAGAGAGCAACCA |
| | GAACAAUUCUAUCAUCAGCGUGCUGUUUGGCACCGGCGAGAAGGAGGAUAGGUCUGUGAAG |
| | CUGCGCAUCACAAAGAAGAUCCUGGAGGCCAUCAGCAACCUGAAGGAGAUCCCAAAGAAUG |
| | UGGCCCCCAUCCAGGAGAUCAUCCUGAAUGUGGCCAAGGCCACCAAGGAGACAUUCAGACA |
| | GGUGUACGCAGGAAACCUGGGAGCACCAUCCACCCUGGAGAAGUUUAUCGCCAAGGACGGC |
| | CAGAAGGAGUUCGAUCUGAAGAAGCUGCAGACAGACCUGAAGAAAGUGAUCCGGGGCAAGU |
| | CUAAGGAGAGAGAUUGGUGCUGUCAGGAGGAGCUGAGGAGCUACGUGGAGCAGAAUACCAU |
| | CCAGUAUGACCUGUGGGCCUGGGGCGAGAUGUUCAACAAGGCCCACACCGCCCUGAAGAUC |
| | AAGUCCACAAGAAACUACAAUUUUGCCAAGCAGAGGCUGGAGCAGUUCAAGGAGAUCCAGU |
| | CUCUGAACAAUCUGCUGGUGGUGAAGAAGCUGAACGACUUUUUCGAUAGCGAGUUUUUCUC |
| | CGGCGAGGAGACCUACACAAUCUGCGUGCACCACCUGGGCGGCAAGGACCUGUCCAAGCUG |
| | UAUAAGGCCUGGGAGGACGAUCCCGCCGAUCCUGAGAAUGCCAUCGUGGUGCUGUGCGACG |
| | AUCUGAAGAACAAUUUUAAGAAGGAGCCUAUCAGGAACAUCCUGCGCUACAUCUUCACCAU |
| | CCGCCAGGAGUGUAGCGCACAGGACAUCCUGGCAGCAGCAAAGUACAAUCAGCAGCUGGAU |
| | CGGUAUAAGAGCCAGAAGGCCAACCCAUCCGUGCUGGGCAAUCAGGGCUUUACCUGGACAA |
| | ACGCCGUGAUCCUGCCAGAGAAGGCCCAGCGGAACGACAGACCCAAUUCUCUGGAUCUGCG |
| | CAUCUGGCUGUACCUGAAGCUGCGGCACCCUGACGGCAGAUGGAAGAAGCACCACAUCCCA |
| | UUCUACGAUACCCGGUUUUUCCAGGAGAUCUAUGCCGCCGGCAAUAGCCCUGUGGACACCU |
| | GUCAGUUUAGGACACCCCGCUUCGGCUAUCACCUGCCUAAGCUGACCGAUCAGACAGCCAU |
| | CCGCGUGAACAAGAAGCACGUGAAGGCAGCAAAGACCGAGGCACGGAUCAGACUGGCCAUC |
| | CAGCAGGGCACACUGCCAGUGUCCAAUCUGAAGAUCACCGAGAUCUCCGCCACAAUCAACU |
| | CUAAGGGCCAGGUGCGCAUCCCCGUGAAGUUUCGGGUGGGAAGGCAGAAGGGAACCCUGCA |
| | GAUCGGCGACCGGUUCUGCGGCUACGAUCAGAACCAGACAGCCUCUCACGCCUAUAGCCUG |
| | UGGGAGGUGGUGAAGGAGGGCCAGUACCACAAGGAGCUGGGCUGUUUUGUGCGCUUCAUCU |
| | CUAGCGGCGACAUCGUGUCCAUCACCGAGAACCGGGGCAAUCAGUUUGAUCAGCUGUCUUA |
| | UGAGGGCCUGGCCUACCCCCAGUAUGCCGACUGGAGAAAGAAGGCCUCCAAGUUCGUGUCU |
| | CUGUGGCAGAUCACCAAGAAGAACAAGAAGAAGGAGAUCGUGACAGUGGAGGCCAAGGAGA |
| | AGUUUGACGCCAUCUGCAAGUACCAGCCUAGGCUGUAUAAGUUCAACAAGGAGUACGCCUA |
| | UCUGCUGCGGGAUAUCGUGAGAGGCAAGAGCCUGGUGGAGCUGCAGCAGAUCAGGCAGGAG |
| | AUCUUUCGCUUCAUCGAGCAGGACUGUGGAGUGACCCGCCUGGGAUCUCUGAGCCUGUCCA |
| | CCCUGGAGACAGUGAAGGCCGUGAAGGGCAUCAUCUACUCCUAUUUUUCUACAGCCCUGAA |
| | UGCCUCUAAGAACAAUCCCAUCAGCGACGAGCAGCGGAAGGAGUUUGAUCCUGAGCUGUUC |
| | GCCCUGCUGGAGAAGCUGGAGCUGAUCAGGACUCGGAAGAAGAAGCAGAAGGUGGAGAGAA |
| | UCGCCAAUAGCCUGAUCCAGACAUGCCUGGAGAACAAUAUCAAGUUCAUCAGGGGCGAGGG |
| | CGACCUGUCCACCACAAACAAUGCCACCAAGAAGAAGGCCAACUCUAGGAGCAUGGAUUGG |
| | CUGGCCAGAGGCGUGUUUAAUAAGAUCCGGCAGCUGGCCCCAAUGCACAACAUCACCCUGU |
| | UCGGCUGCGGCAGCCUGUACACAUCCCACCAGGACCCUCUGGUGCACAGAAACCCAGAUAA |
| | GGCCAUGAAGUGUAGAUGGGCAGCAAUCCCAGUGAAGGACAUCGGCGAUUGGGUGCUGAGA |
| | AAGCUGUCCCAGAACCUGAGGGCCAAGAAUCGGGGCACCGGCGAGUACUAUCACCAGGGCG |
| | UGAAGGAGUUCCUGUCUCACUAUGAGCUGCAGGACCUGGAGGAGGAGCUGCUGAAGUGGCG |
| | GUCUGAUAGAAAGAGCAACAUCCCUUGCUGGGUGCUGCAGAAUAGACUGGCCGAGAAGCUG |
| | GGCAACAAGGAGGCCGUGGUGUACAUCCCAGUGAGGGGCGGCCGCAUCUAUUUUGCAACCC |
| | ACAAGGUGGCAACAGGAGCCGUGAGCAUCGUGUUCGACCAGAAGCAAGUGUGGGUGUGUAA |
| | UGCAGAUCACGUGGCAGCAGCAAACAUCGCACUGACCGGCAAGGGCAUCGGCGAGCAGUCC |
| | UCUGACGAGGAGAACCCCGAUGGCUCCAGGAUCAAGCUGCAGCUGACAUCUAAAAGGCCGG |
| | CGGCCACGAAAAAGGCCGGCCAGGCAAAAAAGAAAAAGUAA (SEQ ID NO: 1089) |
| mRNA | AUGAGCUCCGCCAUCAAGUCCUACAAGUCUGUGCUGCGGCCAAACGAGAGAAAGAAUCAGC |
| corresponding to | UGCUGAAGUCCACCAUCCAGUGCCUGGAGGACGGCUCCGCCUUCUUUUUCAAGAUGCUGCA |
| variant Cas1212 | GGGCCUGUUUGGCGGCAUCACCCCCGAGAUCGUGAGAUUCAGCACAGAGCAGGAGAAGCAG |
| of SEQ ID NO: | CAGCAGGAUAUCGCCCUGUGGUGUGCCGUGAAUUGGUUCAGGCCUGUGAGCCAGGACUCCC |
| 927 | UGACCCACACAAUCGCCUCCGAUAACCUGGUGGAGAAGUUUGAGGAGUACUAUGGCGGCAC |
| | AGCCAGCGACGCCAUCAAGCAGUACUUCAGCGCCUCCAUCGGCGAGUCCUACUAUUGGAAU |
| | GACUGCCGCCAGCAGUACUAUGAUCUGUGUCGGGAGCUGGGCGUGGAGGUGUCUGACCUGA |
| | CCCACGAUCUGGAGAUCCUGUGCCGGGAGAAGUGUCUGGCCGUGGCCACAGAGAGCAACCA |
| | GAACAAUUCUAUCAUCAGCGUGCUGUUUGGCACCGGCGAGAAGGAGGAUAGGUCUGUGAAG |
| | CUGCGCAUCACAAAGAAGAUCCUGGAGGCCAUCAGCAACCUGAAGGAGAUCCCAAAGAAUG |
| | UGGCCCCCAUCCAGGAGAUCAUCCUGAAUGUGGCCAAGGCCACCAAGGAGACAUUCAGACA |
| | GGUGUACGCAGGAAACCUGGGAGCACCAUCCACCCUGGAGAAGUUUAUCGCCAAGGACGGC |
| | CAGAAGGAGUUCGAUCUGAAGAAGCUGCAGACAGACCUGAAGAAAGUGAUCCGGGGCAAGU |
| | CUAAGGAGAGAGAUUGGUGCUGUCAGGAGGAGCUGAGGAGCUACGUGGAGCAGAAUACCAU |
| | CCAGUAUGACCUGUGGGCCUGGGGCGAGAUGUUCAACAAGGCCCACACCGCCCUGAAGAUC |
| | AAGUCCACAAGAAACUACAAUUUUGCCAAGCAGAGGCUGGAGCAGUUCAAGGAGAUCCAGU |
| | CUCUGAACAAUCUGCUGGUGGUGAAGAAGCUGAACGACUUUUUCGAUAGCGAGUUUUUCUC |
| | CGGCGAGGAGACCUACACAAUCUGCGUGCACCACCUGGGCGGCAAGGACCUGUCCAAGCUG |
| | UAUAAGGCCUGGGAGGACGAUCCCGCCGAUCCUGAGAAUGCCAUCGUGGUGCUGUGCGACG |
| | AUCUGAAGAACAAUUUUAAGAAGGAGCCUAUCAGGAACAUCCUGCGCUACAUCUUCACCAU |
| | CCGCCAGGAGUGUAGCGCACAGGACAUCCUGGCAGCAGCAAAGUACAAUCAGCAGCUGGAU |
| | CGGUAUAAGAGCCAGAAGGCCAACCCAUCCGUGCUGGGCAAUCAGGGCUUUACCUGGACAA |
| | ACGCCGUGAUCCUGCCAGAGAAGGCCCAGCGGAACGACAGACCCAAUUCUCUGGAUCUGCG |
| | CAUCUGGCUGUACCUGAAGCUGCGGCACCCUGACGGCAGAUGGAAGAAGCACCACAUCCCA |
| | UUCUACGAUACCCGGUUUUUCCAGGAGAUCUAUGCCGCCGGCAAUAGCCCUGUGGACACCU |
| | GUCAGUUUAGGACACCCCGCUUCGGCUAUCACCUGCCUAAGCUGACCGAUCAGACAGCCAU |
| | CCGCGUGAACAAGAAGCACGUGAAGGCAGCAAAGACCGAGGCACGGAUCAGACUGGCCAUC |
| | CAGCAGGGCACACUGCCAGUGUCCAAUCUGAAGAUCACCGAGAUCUCCGCCACAAUCAACU |
| | CUAAGGGCCAGGUGCGCAUCCCCGUGAAGUUUCGGGUGGGAAGGCAGAAGGGAACCCUGCA |
| | GAUCGGCGACCGGUUCUGCGGCUACGAUCAGAACCAGACAGCCUCUCACGCCUAUAGCCUG |
| | UGGGAGGUGGUGAAGGAGGGCCAGUACCACAAGGAGCUGCGGUGUCGGGUGCGCUUCAUCU |
| | CUAGCGGCGACAUCGUGUCCAUCACCGAGAACCGGGGCAAUCAGUUUGAUCAGCUGUCUUA |
| | UGAGGGCCUGGCCUACCCCCAGUAUGCCGACUGGAGAAAGAAGGCCUCCAAGUUCGUGUCU |
| | CUGUGGCAGAUCACCAAGAAGAACAAGAAGAAGGAGAUCGUGACAGUGGAGGCCAAGGAGA |
| | AGUUUGACGCCAUCUGCAAGUACCAGCCUAGGCUGUAUAAGUUCAACAAGGAGUACGCCUA |
| | UCUGCUGCGGGAUAUCGUGAGAGGCAAGAGCCUGGUGGAGCUGCAGCAGAUCAGGCAGGAG |
| | AUCUUUCGCUUCAUCGAGCAGGACUGUGGAGUGACCCGCCUGGGAUCUCUGAGCCUGUCCA |
| | CCCUGGAGACAGUGAAGGCCGUGAAGGGCAUCAUCUACUCCUAUUUUUCUACAGCCCUGAA |
| | UGCCUCUAAGAACAAUCCCAUCAGCGACGAGCAGCGGAAGGAGUUUGAUCCUGAGCUGUUC |
| | GCCCUGCUGGAGAAGCUGGAGCUGAUCAGGACUCGGAAGAAGAAGCAGAAGGUGGAGAGAA |
| | UCGCCAAUAGCCUGAUCCAGACAUGCCUGGAGAACAAUAUCAAGUUCAUCAGGGGCGAGGG |
| | CGACCUGUCCACCACAAACAAUGCCACCAAGAAGAAGGCCAACUCUAGGAGCAUGGAUUGG |
| | CUGGCCAGAGGCGUGUUUAAUAAGAUCCGGCAGCUGGCCACCAUGCACAACAUCACCCUGU |
| | UCGGCUGCGGCAGCCUGUACACAUCCCACCAGGACCCUCUGGUGCACAGAAACCCAGAUAA |
| | GGCCAUGAAGUGUAGAUGGGCAGCAAUCCCAGUGAAGGACAUCGGCGAUUGGGUGCUGAGA |
| | AAGCUGUCCCAGAACCUGAGGGCCAAGAAUCGGGGCACCGGCGAGUACUAUCACCAGGGCG |
| | UGAAGGAGUUCCUGUCUCACUAUGAGCUGCAGGACCUGGAGGAGGAGCUGCUGAAGUGGCG |
| | GUCUGAUAGAAAGAGCAACAUCCCUUGCUGGGUGCUGCAGAAUAGACUGGCCGAGAAGCUG |
| | GGCAACAAGGAGGCCGUGGUGUACAUCCCAGUGAGGGGCGGCCGCAUCUAUUUUGCAACCC |
| | ACAAGGUGGCAACAGGAGCCGUGAGCAUCGUGUUCGACCAGAAGCAAGUGUGGGUGUGUAA |
| | UGCAGAUCACGUGGCAGCAGCAAACAUCGCACUGACCGGCAAGGGCAUCGGCCGGCAGUCC |
| | UCUGACGAGGAGAACCCCGAUGGCGGCAGGAUCAAGCUGCAGCUGACAUCUAAAAGGCCGG |
| | CGGCCACGAAAAAGGCCGGCCAGGCAAAAAAGAAAAAGUAA (SEQ ID NO: 1090) |

Cas12i2 RNA guides were designed and ordered from Integrated DNA Technologies (IDT) as having 3′ end modified phosphorothioated 2′ O-methyl bases or 5′ end and 3′ end modified phosphorothioated 2′ O-methyl bases guides, as specified in Table 9. Each variant Cas12i2 mRNA was mixed with a crRNA at a 1:1 (Cas12i2:crRNA) volume ratio (1050:1 crRNA:Cas12i2 molar ratio). The mRNA and crRNA were mixed immediately before electroporation. The primary human hepatocyte cells were cultured and electroporated as described in Example 3.

TABLE 9
Chemically modified RNA guide sequences

| RNA guide | Sequence |
|---|---|
| 3′end modified | AGAAAUCCGUCUUUCAUUGACGGCGG |
| E2T5 | AGCAUCCUUGGAUA*mC*mA |
| | *mG (SEQ ID NO: 1091) |
| 5′ and 3′ end | mA*mG*mA*AAUCCGUCUUUCAUUG |
| modified E2T5 | ACGGCGGAGCAUCCUUGGAUA |
| | *mC*mA*mG (SEQ ID NO: 1092) |

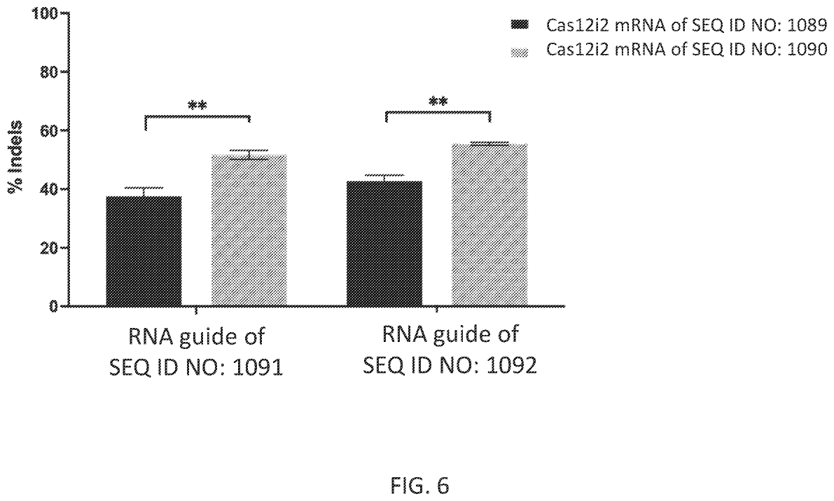

FIG. 6 shows editing of an HAO1 target site by a variant Cas12i2 mRNA and 3′ end modified E2T5 (SEQ ID NO: 1091) or 5′ and 3′ end modified E2T5 (SEQ ID NO: 1092). Indels in the HAO1 target site were introduced following electroporation of the Cas12i2 mRNA of SEQ ID NO: 1089 or SEQ ID NO: 1090 and either the RNA guide of SEQ ID NO: 1091 or SEQ ID NO: 1092. Approximately 50% NGS reads comprised an indel following electroporation of the Cas12i2 mRNA of SEQ ID NO: 1090 and the RNA guide of SEQ ID NO: 1091 or SEQ ID NO: 1092. Statistically significant higher % indels were observed using variant Cas12i2 mRNA of SEQ ID NO: 1090 compared to variant Cas12i2 mRNA of SEQ ID NO: 1089. No statistical difference was observed using 5′ and 3′ versus 3′ only modifications to RNA guide E2T5.

This Example thus shows that HAO1 can be targeted by Cas12i2 mRNA constructs and chemically modified RNA guides in mammalian cells.

### Example 6—Off-Target Analysis of Cas12i2 and HAO1-Targeting RNA Guides

This Example describes on-target versus off-target assessment of a Cas12i2 variant and an HAO1-targeting RNA guide.

HEK293T cells were transfected with a plasmid encoding the variant Cas12i2 of SEQ ID NO: 924 or the variant Cas12i2 of SEQ ID NO: 927 and a plasmid encoding E2T5 (SEQ ID NO: 989), E1T2 (SEQ ID NO: 967), E1T3 (SEQ ID NO: 968), and E2T10 (SEQ ID NO: 994) according to the method described in Example 16 of PCT/US21/25257. The tagmentation-based tag integration site sequencing (TTISS) method described in Example 16 of PCT/US21/25257 was then carried out.

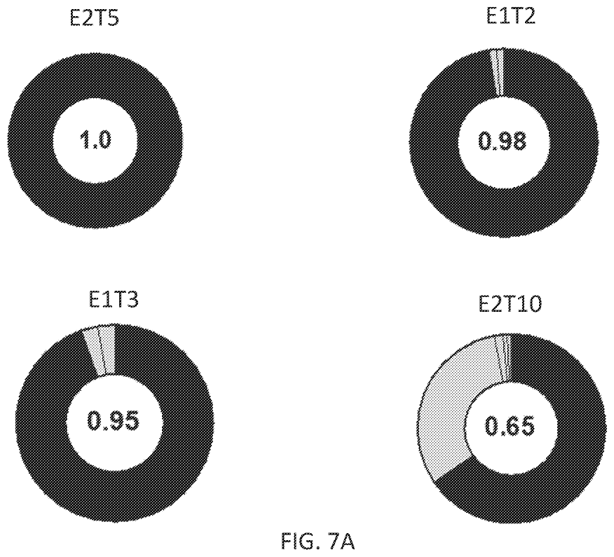

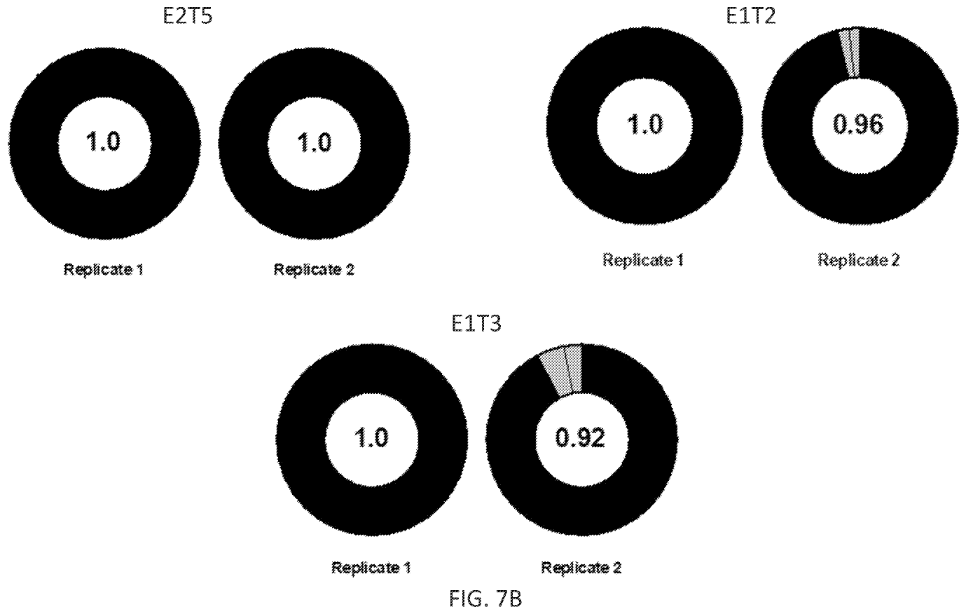

FIG. 7A and FIG. 7B show plots depicting on-target and off-target TTISS reads. The black wedge and centered number represent the fraction of on-target TTISS reads. Each grey wedge represents a unique off-target site identified by TTISS. The size of each grey wedge represents the fraction of TTISS reads mapping to a given off-target site. FIG. 7A shows TTISS reads for variant Cas12i2 of SEQ ID NO: 924, and FIG. 7B shows TTISS reads for variant Cas12i2 of SEQ ID NO: 927.

As shown in FIG. 7A, variant Cas12i2 of SEQ ID NO: 924 paired with E2T5 demonstrated a low likelihood of off-target editing, as 100% of TTISS reads mapped to the on-target. No TTISS reads mapped to potential off-target sites. E1T2 also showed a low likelihood of off-target editing. For E1T2, 98% of TTISS reads mapped to the on-target, and two potential off-target sites represented a combined 2% of TTISS reads. For E5T10, 95% of TTISS reads mapped to the on-target, and two potential off-target sites represented a combined 5% of TTISS reads. E2T10 demonstrated a higher likelihood of off-target editing using the TTISS method. For E2T10, only 65% of TTISS reads mapped to the on-target and 4 potential off-target sites represented the remaining combined 35% of TTISS reads. One potential off-target represented the majority of potential off-target TTISS reads for E2T10.

As shown in FIG. 7B, variant Cas12i2 of SEQ ID NO: 927 paired with E2T5 demonstrated a low likelihood of off-target editing, as 100% of TTISS reads mapped to the on-target. No TTISS reads mapped to potential off-target sites. Variant Cas12i2 of SEQ ID NO: 927 paired with the E1T2 or E1T3 also demonstrated a low likelihood of off-target editing. For E1T2, 100% of TTISS reads in replicate 1 and 96% of TTISS reads in replicate 2 mapped to the on-target; two potential off-target sites represented the remaining 4% of TTISS reads in replicate 2. For E1T3, 100% of TTISS reads in replicate 1 and 92% of TTISS reads in replicate 2 mapped to the on-target; two potential off-target sites represented the remaining 8% of TTISS reads in replicate 2.

Therefore, this Example shows that compositions comprising Cas12i2 and HAO1-targeting RNA guides comprise different off-target activity profiles.

### Example 7—HAO1 Protein Knockdown with Cas12i2 and HAO1-Targeting RNA Guides

This Example describes use of a Western Blot to identify knockdown of HAO1 protein using variant Cas12i2 of SEQ ID NO: 924 and HAO1-targeting RNA guides.

Primary hepatocyte cells from human donors were thawed from liquid nitrogen very quickly in a 37° ° C. water bath. The cells were added to pre-warmed hepatocyte recovery media (Thermo Fisher, CM7000) and centrifuged at 100 g for 10 minutes. The cell pellet was resuspended in appropriate volume of hepatocyte plating Medium (Williams' Medium E, Thermo Fisher A1217601 supplemented with Hepatocyte Plating Supplement Pack (serum-containing), Thermo Fisher CM3000). The cells were subjected to trypan blue viability count with an Inucyte disposable hemocytometer (Fisher scientific, 22-600-100). The cells were then washed in PBS and resuspended in P3 buffer+supplement (Lonza, VXP-3032) at a concentration of ˜5000 cells/μL. Resuspended cells were dispensed at 500,000 cells/reaction into Lonza electroporation cuvettes

For the RNP reactions, E2T5 (SEQ ID NO: 989) was used as the HAO1-targeting RNA guides. RNPs were added to each reaction at a final concentration of 20 μM (Cas12i2), and transfection enhancer oligos were then added at a final concentration of 4 μM. Unelectroporated cells and cells electroporated without cargo were used as negative controls.

The strips were electroporated using an electroporation device (program CA137, Lonza 4D-nucleofector). Immediately following electroporation, pre-warmed Hepatocyte plating medium was added to each well and mixed very gently by pipetting. For each technical replicate plate, 500,000 cells of diluted nucleofected cells were plated into a pre-warmed collagen-coated 24-well plate (Thermo Fisher) with wells containing Hepatocyte plating medium. The cells were then incubated at 37° C. The media was changed to hepatocyte maintenance media (Williams' Medium E, Thermo Fisher A1217601 supplemented with William's E medium Cell Maintenance Cocktail, Thermo Fisher CM 4000) after the cells attached after 24 hours. Fresh hepatocyte maintenance media was replaced every 48 hours.

16 days post RNP electroporation, the media was aspirated, and the cells were washed gently with PBS. Cells were then lysed with RIPA Lysis and Extraction buffer (Thermo Fisher 89901)+1× protease inhibitors (Thermo Fisher 78440) for 30 minutes on ice, mixing the samples every 5 minutes. Cell lysate was quantified via Pierce BCA Protein Assay Kit (Thermo Fisher 23227). 15 μg of total protein per sample was prepared for SDS-PAGE in 1× Laemmlli Sample buffer (BioRad 1610747) and 100 mM DTT, then heated at 95 C for 10 minutes. Samples were run on a 4-15% TGX gel (BioRad 5671084) at 200V for 45 minutes. Samples were transferred to a 0.2 um nitrocellulose membrane (BioRad 1704159) using the Trans Blot Turbo System. The membrane was blocked in Intercept TBS Blocking Buffer (Li-cor 927-60001) for 30 minutes at room temperature. The blot was then incubated in a 1:1000 dilution of primary anti-HAO1 antibody (Genetex GTX81144) and 1:2500 dilution of primary anti-vinculin antibody (Sigma V9131) in blocking buffer at 4 C overnight. The blot was washed three times with TBST (ThermoFisher 28360) for 5 minutes each, then incubated with a 1:12500 dilution of IR680 anti-mouse (ThermoFisher PI35518) and IR800 anti-rabbit secondary antibodies (ThermoFisher PISA535571) in TBST for 1 hour at room temperature. The blot was then washed three times with TBST for 5 minutes each and visualized on the Li-cor Odyssey CLX.

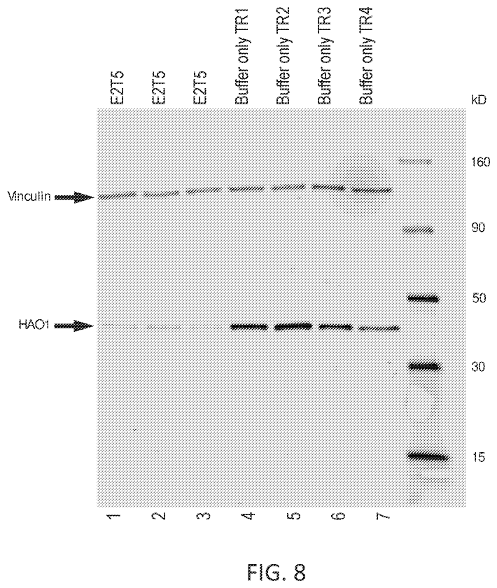

Knockdown of HAO1 protein was observed in primary human hepatocytes at Day 7 post editing by Cas12i2 RNPs targeting the HAO1 gene with E2T5 (lanes 1-3 of FIG. 8). HAO1 knockdown was not observed for the buffer only controls (lanes 4-7).

This Example thus shows that HAO1 protein levels were decreased following editing with Cas12i2 and HAO1-targeting RNA guides.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of.” “only one of.” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B.” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. A gene editing system for genetic editing of a hydroxyacid oxidase 1 (HAO1) gene, comprising
(i) a Cas12i2 polypeptide or a first nucleic acid encoding the Cas12i2 polypeptide, wherein the Cas12i2 polypeptide comprises an amino acid sequence at least 95% identical to SEQ ID NO: 922 and comprises one or more mutations relative to SEQ ID NO: 922;
(ii) an RNA guide or a second nucleic acid encoding the RNA guide, wherein the RNA guide comprises a spacer sequence specific to a target sequence within an HAO1 gene, the target sequence being adjacent to a protospacer adjacent motif (PAM) comprising the motif of 5′-TTN-3′, which is located 5′ to the target sequence.

2. The gene editing system of claim 1, wherein the one or more mutations in the Cas12i2 polypeptide are at positions D581, G624, F626, P868, I926, V1030, E1035, and/or S1046 of SEQ ID NO: 922.

3. The gene editing system of claim 1, wherein the one or more mutations are amino acid substitutions, which optionally is D581R, G624R, F626R, P868T, I926R, V1030G, E1035R, S1046G, or a combination thereof.

4. The gene editing gene editing system of claim 3, wherein the Cas12i2 polypeptide comprises:
(i) mutations at positions D581, D911, I926, and V1030, which optionally are amino acid substitutions of D581R, D911R, I926R, and V1030G;
(ii) mutations at positions D581, I926, and V1030, which optionally are amino acid substitutions of D581R, I926R, and V1030G;
(iii) mutations at positions D581, I926, V1030, and S1046, which optionally are amino acid substitutions of D581R, I926R, V1030G, and S1046G;
(iv) mutations at positions D581, G624, F626, I926, V1030, E1035, and S1046, which optionally are amino acid substitutions of D581R, G624R, F626R, I926R, V1030G, E1035R, and S1046G; or
(v) mutations at positions D581, G624, F626, P868, I926, V1030, E1035, and S1046, which optionally are amino acid substitutions of D581R, G624R, F626R, P868T, I926R, V1030G, E1035R, and S1046G.

5. The gene editing system of claim 1, wherein the Cas12i2 polypeptide comprises the amino acid sequence of SEQ ID NO: 923, 924, 925, 926, or 927, optionally wherein the Cas12i2 polypeptide comprises the amino acid sequence of SEQ ID NO: 924 or 927.

6. The gene editing system of claim 1, which comprises the first nucleic acid encoding the Cas12i2 polypeptide.

7. The gene editing system of claim 6, wherein the first nucleic acid is a messenger RNA (mRNA).

8. The gene editing system of claim 7, wherein the first nucleic acid is included in a viral vector, which optionally is an adeno-associated viral (AAV) vector.

9. The gene editing system of claim 1, wherein the target sequence is within exon 1 or exon 2 of the HAO1 gene.

10. The gene editing system of claim 9, wherein the target sequence comprises:
(i)
(SEQ ID NO: 1025)
5′-CAAAGTCTATATATGACTAT-3′;
(ii)
(SEQ ID NO: 1026)
5′-GGAAGTACTGATTTAGCATG-3′;
(iii)
(SEQ ID NO: 1046)
5′-TAGATGGAAGCTGTATCCAA-3′;
(iv)
(SEQ ID NO: 1047)
5′-CGGAGCATCCTTGGATACAG-3′;
or
(v)
(SEQ ID NO: 1052)
5′-AGGACAGAGGGTCAGCATGC-3.

11. The system of claim 10, wherein the spacer sequence comprises:
(i)
(SEQ ID NO: 1093
5′-CAAAGUCUAUAUAUGACUAU-3′;
(ii)
(SEQ ID NO: 1094)
5′-GGAAGUACUGAUUUAGCAUG-3′;
(iii)
(SEQ ID NO: 1095)
5′-UAGAUGGAAGCUGUAUCCAA-3′;
(iv)
(SEQ ID NO: 1096)
5′-CGGAGCAUCCUUGGAUACAG-3′;
or
(v)
(SEQ ID NO: 1097)
5′-AGGACAGAGGGUCAGCAUGC-3.

12. The gene editing system of claim 1, wherein the spacer sequence is 20-30-nucleotide in length, optionally wherein the spacer is 20-nucleotide in length.

13. The gene editing system of claim 1, wherein the RNA guide comprises the spacer and a direct repeat sequence.

14. The gene editing system of claim 13, wherein the direct repeat sequence is 23-36-nucleotide in length.

15. The gene editing system of claim 14, wherein the direct repeat sequence is at least 90% identical to any one of SEQ ID NOs: 1-10 or a fragment thereof that is at least 23-nucleotide in length.

16. The gene editing system of claim 15, wherein the direct repeat sequence is any one of SEQ ID NOs: 1-10, or a fragment thereof that is at least 23-nucleotide in length.

17. The gene editing system of claim 16, wherein the direct repeat sequence is 5′-AGAAAUCCGUCUUUCAUUGACGG-3′ (SEQ ID NO: 10).

18. The gene editing system of claim 1, wherein the RNA guide comprises the nucleotide sequence of:
(i)
(SEQ ID NO: 967)
5′-AGAAAUCCGUCUUUCAUUGAC
GGCAAAGUCUAUAUAUGACUAU-3′;
(ii)
(SEQ ID NO: 968)
5′-AGAAAUCCGUCUUUCAUUGACG
GGGAAGUACUGAUUUAGCAUG-3′;
(iii)
(SEQ ID NO: 988)
5′-AGAAAUCCGUCUUUCAUUGACG
GUAGAUGGAAGCUGUAUCCAA-3′;
(iv)
(SEQ ID NO: 989)
5′-AGAAAUCCGUCUUUCAUUGACG
GCGGAGCAUCCUUGGAUACAG-3′;
or
(v)
(SEQ ID NO: 994)
5′-AGAAAUCCGUCUUUCAUUGACG
GAGGACAGAGGGUCAGCAUGC-3′.

19. The gene editing system of claim 1, wherein the system comprises the second nucleic acid encoding the RNA guide.

20. The gene editing system of claim 19, wherein the nucleic acid encoding the RNA guide is located in a viral vector.

21. The gene editing system of claim 7, wherein the viral vector comprises the both the first nucleic acid encoding the Cas12i2 polypeptide and the second nucleic acid encoding the RNA guide.

22. The gene editing system of claim 1, wherein the system comprises the first nucleic acid encoding the Cas12i2 polypeptide, which is located in a first vector, and wherein the system comprises the second nucleic acid encoding the RNA guide, which is located in a second vector; optionally wherein the first and/or the second vector is a viral vector.

23. The gene editing system of claim 22, wherein the first and second vector are the same vector.

24. The gene editing system of claim 1, wherein the system comprises one or more lipid nanoparticles (LNPs), which encompass (i), (ii), or both.

25. The gene editing system of claim 24, wherein the system comprises the LNP, which encompass (i), and wherein the system comprises a viral vector comprising the second nucleic acid encoding the RNA guide; optionally wherein the viral vector is an AAV vector.

26. The gene editing system of claim 24, wherein the system comprises the LNP, which encompass (ii), and wherein the system comprises a viral vector comprising the first nucleic acid encoding Cas12i2 polypeptide; optionally wherein the viral vector is an AAV vector.

27. A gene editing system for genetic editing of a hydroxyacid oxidase 1 (HAO1) gene, comprising
(i) a Cas12i polypeptide or a first nucleic acid encoding the Cas12i polypeptide, optionally wherein the Cas12i polypeptide is a Cas12i2 polypeptide;
(ii) an RNA guide or a second nucleic acid encoding the RNA guide, wherein the RNA guide comprises a spacer sequence specific to a target sequence within exon 1 or exon 2 of an HAO1 gene, the target sequence being adjacent to a protospacer adjacent motif (PAM) comprising the motif of 5′-TTN-3′, which is located 5′ to the target sequence.

28. The gene editing system of claim 27, wherein the target sequence comprises:
(i)
(SEQ ID NO: 1025)
5′-CAAAGTCTATATATGACTAT-3′;
(ii)
(SEQ ID NO: 1026)
5′-GGAAGTACTGATTTAGCATG-3′;
(iii)
(SEQ ID NO: 1046)
5′-TAGATGGAAGCTGTATCCAA-3′;
(iv)
(SEQ ID NO: 1047)
5′-CGGAGCATCCTTGGATACAG-3′;
or
(v)
(SEQ ID NO: 1052)
5′-AGGACAGAGGGTCAGCATGC-3.

29. The gene editing system of claim 27, wherein the spacer sequence comprises:
(i)
(SEQ ID NO: 1093)
5′-CAAAGUCUAUAUAUGACUAU-3′;
(ii)
(SEQ ID NO: 1094)
5′-GGAAGUACUGAUUUAGCAUG-3′;
(iii)
(SEQ ID NO: 1095)
5′-UAGAUGGAAGCUGUAUCCAA-3′;
(iv)
(SEQ ID NO: 1096)
5′-CGGAGCAUCCUUGGAUACAG-3′;
or
(v)
(SEQ ID NO: 1097)
5′-AGGACAGAGGGUCAGCAUGC-3

30. The gene editing system of claim 27, which comprises the first nucleic acid encoding the Cas12i polypeptide.

31. The gene editing system of claim 30, wherein the first nucleic acid is a messenger RNA (mRNA).

32. The gene editing system of claim 30, wherein the first nucleic acid is included in a viral vector, which optionally is an adeno-associated viral (AAV) vector.

33. The gene editing system of claim 27, wherein the spacer is 20-30-nucleotide in length, optionally wherein the spacer is 20-nucleotide in length.

34. The gene editing system of claim 27, wherein the RNA guide comprises the spacer sequence and a direct repeat sequence.

35. The gene editing system of claim 34, wherein the direct repeat sequence is 23-36-nucleotide in length.

36. The gene editing system of claim 35, wherein the direct repeat sequence is at least 90% identical to any one of SEQ ID NOs: 1-10 or a fragment thereof that is at least 23-nucleotide in length.

37. The gene editing system of claim 36, wherein the direct repeat sequence is any one of SEQ ID NOs: 1-10, or a fragment thereof that is at least 23-nucleotide in length.

38. The gene editing system of claim 37, wherein the direct repeat sequence is 5′-AGAAAUCCGUCUUUCAUUGACGG-3′ (SEQ ID NO: 10).

39. The gene editing system of claim 34, wherein the RNA guide comprises the nucleotide sequence of:
(i)
(SEQ ID NO: 967)
5′-AGAAAUCCGUCUUUCAUUGAC
GGCAAAGUCUAUAUAUGACUAU-3′;
(ii)
(SEQ ID NO: 968)
5′-AGAAAUCCGUCUUUCAUUGAC
GGGGAAGUACUGAUUUAGCAUG-3′;
(iii)
(SEQ ID NO: 988)
5′-AGAAAUCCGUCUUUCAUUGAC
GGUAGAUGGAAGCUGUAUCCAA-3′;
(iv)
(SEQ ID NO: 989)
5′-AGAAAUCCGUCUUUCAUUGAC
GGCGGAGCAUCCUUGGAUACAG-3′;
or
(v)
(SEQ ID NO: 994)
5′-AGAAAUCCGUCUUUCAUUGAC
GGAGGACAGAGGGUCAGCAUGC-3′.

40. The gene editing system of claim 27, wherein the system comprises the second nucleic acid encoding the RNA guide.

41. The gene editing system of claim 40, wherein the nucleic acid encoding the RNA guide is located in a viral vector.

42. The gene editing system of claim 32, wherein the viral vector comprises the both the first nucleic acid encoding the Cas12i2 polypeptide and the second nucleic acid encoding the RNA guide.

43. The gene editing system of claim 27, wherein the system comprises the first nucleic acid encoding the Cas12i2 polypeptide, which is located in a first vector, and wherein the system comprises the second nucleic acid encoding the RNA guide, which is located in a second vector.

44. The gene editing system of claim 27, wherein the system comprises one or more lipid nanoparticles (LNPs), which encompass (i), (ii), or both.

45. The gene editing system of claim 44, wherein the system comprises the LNP, which encompass (i), and wherein the system comprises a viral vector comprising the second nucleic acid encoding the RNA guide; optionally wherein the viral vector is an AAV vector.

46. The gene editing system of claim 44, wherein the system comprises the LNP, which encompass (ii), and wherein the system comprises a viral vector comprising the first nucleic acid encoding Cas12i2 polypeptide; optionally wherein the viral vector is an AAV vector.

47. A pharmaceutical composition comprising the gene editing system set forth in claim 1.

48. A kit comprising the elements (i) and (ii) of the gene editing system set forth in claim 1.

49. A method for editing a hydroxyacid oxidase 1 (HAO1) gene in a cell, the method comprising contacting a host cell with the gene editing system for editing the HAO1 gene set forth in claim 1 to genetically edit the HAO1 gene in the host cell.

50. The method of claim 49, wherein the host cell is cultured in vitro.

51. The method of claim 49, wherein contacting step is performed by administering the system for editing the HAO1 gene to a subject comprising the host cell.

52. A cell comprising a disrupted hydroxyacid oxidase 1 (HAO1) gene, wherein the cell optionally is produced by contacting a host cell with the gene editing system of claim 1 to genetically edit the HAO1 gene in the host cell, thereby disrupting the HAO1 gene.

53. A method for treating primary hyperoxaluria (PH) in a subject, comprising administering to a subject in need thereof a gene editing system for editing a hydroxyacid oxidase 1 (HAO1) gene set forth in claim 1 or a cell comprising a disrupted HAO1 gene generated by the gene editing system.

54. The method of claim 53, wherein the subject is a human patient having the PH, which optionally is PH1, PH2, or PH3.

55. The method of claim 54, wherein the PH is PH1.

56. An RNA guide, comprising (i) a spacer sequence that is specific to a target sequence in a hydroxyacid oxidase 1 (HAO1) gene, wherein the target sequence is adjacent to a protospacer adjacent motif (PAM) comprising the motif of 5′-TTN-3′, which is located 5′ to the target sequence; and (ii) a direct repeat sequence.

57. The RNA guide of claim 56, wherein the spacer is 20-30-nucleotide in length, optionally 20-nucleotide in length.

58. The RNA guide of claim 56, wherein the direct repeat sequence is 23-36-nucleotide in length, optionally 23-nucleotide in length.

59. The RNA guide of claim 56, wherein the target sequence is within exon 1 or exon 2 of the HAO1 gene.

60. The RNA guide of claim 59, wherein the target sequence comprises:
(i)
(SEQ ID NO: 1025)
5′-CAAAGTCTATATATGACTAT-3′;
(ii)
(SEQ ID NO: 1026)
5′-GGAAGTACTGATTTAGCATG-3′;
(iii)
(SEQ ID NO: 1046)
5′-TAGATGGAAGCTGTATCCAA-3′;
(iv)
(SEQ ID NO: 1047)
5′-CGGAGCATCCTTGGATACAG-3′;
or
(v)
(SEQ ID NO: 1052)
5′-AGGACAGAGGGTCAGCATGC-3

61. The RNA guide of claim 60, wherein the spacer sequence comprises:
(i)
(SEQ ID NO: 1093)
5′-CAAAGUCUAUAUAUGACUAU-3′;
(ii)
(SEQ ID NO: 1094)
5′-GGAAGUACUGAUUUAGCAUG-3′;
(iii)
(SEQ ID NO: 1095)
5′-UAGAUGGAAGCUGUAUCCAA-3′;
(iv)
(SEQ ID NO: 1096)
5′-CGGAGCAUCCUUGGAUACAG-3′;
or
(v)
(SEQ ID NO: 1097)
5′-AGGACAGAGGGUCAGCAUGC-3

62. The RNA guide of claim 56, wherein the direct repeat sequence is at least 90% identical to any one of SEQ ID NOs: 1-10 or a fragment thereof that is at least 23-nucleotide in length.

63. The RNA guide of claim 62, wherein the direct repeat sequence is any one of SEQ ID NOs: 1-10, or a fragment thereof that is at least 23-nucleotide in length.

64. The RNA guide of claim 63, wherein the direct repeat sequence is 5′-AGAAAUCCGUCUUUCAUUGACGG-3′ (SEQ ID NO: 10).

65. The RNA guide of claim 56, which comprises the nucleotide sequence of:
(i)
(SEQ ID NO: 967)
5′-AGAAAUCCGUCUUUCAUUGACG
GCAAAGUCUAUAUAUGACUAU-3′;
(ii)
(SEQ ID NO: 968)
5′-AGAAAUCCGUCUUUCAUUGACG
GGGAAGUACUGAUUUAGCAUG-3′;
(iii)
(SEQ ID NO: 988)
5′-AGAAAUCCGUCUUUCAUUGACG
GUAGAUGGAAGCUGUAUCCAA-3′;
(iv)
(SEQ ID NO: 989)
5′-AGAAAUCCGUCUUUCAUUGAC
GGCGGAGCAUCCUUGGAUACAG-3′;
or
(v)
(SEQ ID NO: 994)
5′-AGAAAUCCGUCUUUCAUUGAC
GGAGGACAGAGGGUCAGCAUGC-3′.


SEQUENCE LISTING (1097 sequences)

SEQ ID NO: 1 - RNA, 36 nt, Artificial Sequence
  Synthetic
guugcaaaacccaagaaauccgucuuucauugacgg

SEQ ID NO: 2 - RNA, 36 nt, Artificial Sequence
  Synthetic
aauagcggcccuaagaaauccgucuuucauugacgg

SEQ ID NO: 3 - RNA, 36 nt, Artificial Sequence
  Synthetic
auuggaacuggcgagaaauccgucuuucauugacgg

SEQ ID NO: 4 - RNA, 36 nt, Artificial Sequence
  Synthetic
ccagcaacaccuaagaaauccgucuuucauugacgg

SEQ ID NO: 5 - RNA, 36 nt, Artificial Sequence
  Synthetic
cggcgcucgaauaggaaauccgucuuucauugacgg

SEQ ID NO: 6 - RNA, 36 nt, Artificial Sequence
  Synthetic
guggcaacaccuaagaaauccgucuuucauugacgg

SEQ ID NO: 7 - RNA, 36 nt, Artificial Sequence
  Synthetic
guugcaacaccuaagaaauccgucuuucauugacgg

SEQ ID NO: 8 - RNA, 36 nt, Artificial Sequence
  Synthetic
guugcaaugccuaagaaauccgucuuucauugacgg

SEQ ID NO: 9 - RNA, 34 nt, Artificial Sequence
  Synthetic
gcaacaccuaagaaauccgucuuucauugacggg

SEQ ID NO: 10 - RNA, 23 nt, Artificial Sequence
  Synthetic
agaaauccgucuuucauugacgg

SEQ ID NO: 11 - DNA, 30 bp, Artificial Sequence
  Synthetic
cctggaaaatgctgcaatattatcagccaa

SEQ ID NO: 12 - DNA, 30 bp, Artificial Sequence
  Synthetic
tcttacctggaaaatgctgcaatattatca

SEQ ID NO: 13 - DNA, 30 bp, Artificial Sequence
  Synthetic
cttacctggaaaatgctgcaatattatcag

SEQ ID NO: 14 - DNA, 30 bp, Artificial Sequence
  Synthetic
ttacctggaaaatgctgcaatattatcagc

SEQ ID NO: 15 - DNA, 30 bp, Artificial Sequence
  Synthetic
tcagccaaagtttcttcatcatttgcccca

SEQ ID NO: 16 - DNA, 30 bp, Artificial Sequence
  Synthetic
cttcatcatttgccccagacctgtaatagt

SEQ ID NO: 17 - DNA, 30 bp, Artificial Sequence
  Synthetic
ttcatcatttgccccagacctgtaatagtc

SEQ ID NO: 18 - DNA, 30 bp, Artificial Sequence
  Synthetic
atcatttgccccagacctgtaatagtcata

SEQ ID NO: 19 - DNA, 30 bp, Artificial Sequence
  Synthetic
gccccagacctgtaatagtcatatatagac

SEQ ID NO: 20 - DNA, 30 bp, Artificial Sequence
  Synthetic
ccccagacctgtaatagtcatatatagact

SEQ ID NO: 21 - DNA, 30 bp, Artificial Sequence
  Synthetic
aaaaaataaattttcttacctggaaaatgc

SEQ ID NO: 22 - DNA, 30 bp, Artificial Sequence
  Synthetic
ggaagtactgatttagcatgttgttcataa

SEQ ID NO: 23 - DNA, 30 bp, Artificial Sequence
  Synthetic
agcatgttgttcataatcattgatacaaat

SEQ ID NO: 24 - DNA, 30 bp, Artificial Sequence
  Synthetic
gcatgttgttcataatcattgatacaaatt

SEQ ID NO: 25 - DNA, 30 bp, Artificial Sequence
  Synthetic
ttcataatcattgatacaaattagccgggg

SEQ ID NO: 26 - DNA, 30 bp, Artificial Sequence
  Synthetic
ataatcattgatacaaattagccgggggag

SEQ ID NO: 27 - DNA, 30 bp, Artificial Sequence
  Synthetic
atacaaattagccgggggagcattttcaca

SEQ ID NO: 28 - DNA, 30 bp, Artificial Sequence
  Synthetic
gccgggggagcattttcacaggttattgct

SEQ ID NO: 29 - DNA, 30 bp, Artificial Sequence
  Synthetic
tcacaggttattgctatcccagatggagtt

SEQ ID NO: 30 - DNA, 30 bp, Artificial Sequence
  Synthetic
cacaggttattgctatcccagatggagttc

SEQ ID NO: 31 - DNA, 30 bp, Artificial Sequence
  Synthetic
acaggttattgctatcccagatggagttcg

SEQ ID NO: 32 - DNA, 30 bp, Artificial Sequence
  Synthetic
gaagtactgatttagcatgttgttcataat

SEQ ID NO: 33 - DNA, 30 bp, Artificial Sequence
  Synthetic
taaaaaataaattttcttacctggaaaatg

SEQ ID NO: 34 - DNA, 30 bp, Artificial Sequence
  Synthetic
aaaaataaattttcttacctggaaaatgct

SEQ ID NO: 35 - DNA, 30 bp, Artificial Sequence
  Synthetic
aaaacatgattttaaaaaataaattttctt

SEQ ID NO: 36 - DNA, 30 bp, Artificial Sequence
  Synthetic
tatcaatgattatgaacaacatgctaaatc

SEQ ID NO: 37 - DNA, 30 bp, Artificial Sequence
  Synthetic
tgaacaacatgctaaatcagtacttccaaa

SEQ ID NO: 38 - DNA, 30 bp, Artificial Sequence
  Synthetic
caaagtctatatatgactattacaggtctg

SEQ ID NO: 39 - DNA, 30 bp, Artificial Sequence
  Synthetic
caggtctggggcaaatgatgaagaaacttt

SEQ ID NO: 40 - DNA, 30 bp, Artificial Sequence
  Synthetic
ggctgataatattgcagcattttccaggta

SEQ ID NO: 41 - DNA, 30 bp, Artificial Sequence
  Synthetic
gctgataatattgcagcattttccaggtaa

SEQ ID NO: 42 - DNA, 30 bp, Artificial Sequence
  Synthetic
cagcattttccaggtaagaaaatttatttt

SEQ ID NO: 43 - DNA, 30 bp, Artificial Sequence
  Synthetic
tccaggtaagaaaatttattttttaaaatc

SEQ ID NO: 44 - DNA, 30 bp, Artificial Sequence
  Synthetic
ccaggtaagaaaatttattttttaaaatca

SEQ ID NO: 45 - DNA, 30 bp, Artificial Sequence
  Synthetic
aaacatgattttaaaaaataaattttctta

SEQ ID NO: 46 - DNA, 30 bp, Artificial Sequence
  Synthetic
attttttaaaatcatgttttaaaattacac

SEQ ID NO: 47 - DNA, 30 bp, Artificial Sequence
  Synthetic
caggtaagaaaatttattttttaaaatcat

SEQ ID NO: 48 - DNA, 30 bp, Artificial Sequence
  Synthetic
tttaaaatcatgttttaaaattacacaaag

SEQ ID NO: 49 - DNA, 30 bp, Artificial Sequence
  Synthetic
ttaaaatcatgttttaaaattacacaaaga

SEQ ID NO: 50 - DNA, 30 bp, Artificial Sequence
  Synthetic
taaaatcatgttttaaaattacacaaagac

SEQ ID NO: 51 - DNA, 30 bp, Artificial Sequence
  Synthetic
aaaatcatgttttaaaattacacaaagacc

SEQ ID NO: 52 - DNA, 30 bp, Artificial Sequence
  Synthetic
aaatcatgttttaaaattacacaaagaccg

SEQ ID NO: 53 - DNA, 30 bp, Artificial Sequence
  Synthetic
gtgtaattttaaaacatgattttaaaaaat

SEQ ID NO: 54 - DNA, 30 bp, Artificial Sequence
  Synthetic
tgtaattttaaaacatgattttaaaaaata

SEQ ID NO: 55 - DNA, 30 bp, Artificial Sequence
  Synthetic
taaaacatgattttaaaaaataaattttct

SEQ ID NO: 56 - DNA, 30 bp, Artificial Sequence
  Synthetic
ttttttaaaatcatgttttaaaattacaca

SEQ ID NO: 57 - DNA, 30 bp, Artificial Sequence
  Synthetic
gtatcaatgattatgaacaacatgctaaat

SEQ ID NO: 58 - DNA, 24 bp, Artificial Sequence
  Synthetic
ttgctatcccagatggagttcgtt

SEQ ID NO: 59 - DNA, 21 bp, Artificial Sequence
  Synthetic
ctatcccagatggagttcgtt

SEQ ID NO: 60 - DNA, 30 bp, Artificial Sequence
  Synthetic
ttttttaattctagatggaagctgtatcca

SEQ ID NO: 61 - DNA, 30 bp, Artificial Sequence
  Synthetic
attttattttttaattctagatggaagctg

SEQ ID NO: 62 - DNA, 30 bp, Artificial Sequence
  Synthetic
attttttaattctagatggaagctgtatcc

SEQ ID NO: 63 - DNA, 30 bp, Artificial Sequence
  Synthetic
tattttttaattctagatggaagctgtatc

SEQ ID NO: 64 - DNA, 30 bp, Artificial Sequence
  Synthetic
ttttattttttaattctagatggaagctgt

SEQ ID NO: 65 - DNA, 30 bp, Artificial Sequence
  Synthetic
aaaaataaaataaaataaaaggctttagag

SEQ ID NO: 66 - DNA, 30 bp, Artificial Sequence
  Synthetic
attttattttattttttaattctagatgga

SEQ ID NO: 67 - DNA, 30 bp, Artificial Sequence
  Synthetic
tattttattttattttttaattctagatgg

SEQ ID NO: 68 - DNA, 30 bp, Artificial Sequence
  Synthetic
tgaaactctaaagccttttattttatttta

SEQ ID NO: 69 - DNA, 30 bp, Artificial Sequence
  Synthetic
tttaattctagatggaagctgtatccaagg

SEQ ID NO: 70 - DNA, 30 bp, Artificial Sequence
  Synthetic
ttttattttattttttaattctagatggaa

SEQ ID NO: 71 - DNA, 30 bp, Artificial Sequence
  Synthetic
ttaattctagatggaagctgtatccaagga

SEQ ID NO: 72 - DNA, 30 bp, Artificial Sequence
  Synthetic
tattttattttttaattctagatggaagct

SEQ ID NO: 73 - DNA, 30 bp, Artificial Sequence
  Synthetic
catctagaattaaaaaataaaataaaataa

SEQ ID NO: 74 - DNA, 30 bp, Artificial Sequence
  Synthetic
taattctagatggaagctgtatccaaggat

SEQ ID NO: 75 - DNA, 30 bp, Artificial Sequence
  Synthetic
aattctagatggaagctgtatccaaggatg

SEQ ID NO: 76 - DNA, 30 bp, Artificial Sequence
  Synthetic
attctagatggaagctgtatccaaggatgc

SEQ ID NO: 77 - DNA, 30 bp, Artificial Sequence
  Synthetic
tagatggaagctgtatccaaggatgctccg

SEQ ID NO: 78 - DNA, 30 bp, Artificial Sequence
  Synthetic
ctgaaacagatctgtcgacttctgttttag

SEQ ID NO: 79 - DNA, 30 bp, Artificial Sequence
  Synthetic
taggacagagggtcagcatgccaatatgtg

SEQ ID NO: 80 - DNA, 30 bp, Artificial Sequence
  Synthetic
aggacagagggtcagcatgccaatatgtgt

SEQ ID NO: 81 - DNA, 30 bp, Artificial Sequence
  Synthetic
ggacagagggtcagcatgccaatatgtgtg

SEQ ID NO: 82 - DNA, 30 bp, Artificial Sequence
  Synthetic
ccactgtgagaggtaggaggaagattgtca

SEQ ID NO: 83 - DNA, 30 bp, Artificial Sequence
  Synthetic
tgttttaggacagagggtcagcatgccaat

SEQ ID NO: 84 - DNA, 30 bp, Artificial Sequence
  Synthetic
gcctccttctgtccctgtggtgacaatctt

SEQ ID NO: 85 - DNA, 30 bp, Artificial Sequence
  Synthetic
tcaccacagggacagaaggaggctaacgtt

SEQ ID NO: 86 - DNA, 30 bp, Artificial Sequence
  Synthetic
cggagcatccttggatacagcttccatcta

SEQ ID NO: 87 - DNA, 30 bp, Artificial Sequence
  Synthetic
agcaacattccggagcatccttggatacag

SEQ ID NO: 88 - DNA, 30 bp, Artificial Sequence
  Synthetic
cagcaacattccggagcatccttggataca

SEQ ID NO: 89 - DNA, 30 bp, Artificial Sequence
  Synthetic
gatacagcttccatctagaattaaaaaata

SEQ ID NO: 90 - DNA, 30 bp, Artificial Sequence
  Synthetic
ctcctacctctcacagtggcaagctcgccg

SEQ ID NO: 91 - DNA, 30 bp, Artificial Sequence
  Synthetic
tgtccctgtggtgacaatcttcctcctacc

SEQ ID NO: 92 - DNA, 30 bp, Artificial Sequence
  Synthetic
gcatgctgaccctctgtcctaaaacagaag

SEQ ID NO: 93 - DNA, 30 bp, Artificial Sequence
  Synthetic
cctgggcaaccgtctggatgatgtgcgtaa

SEQ ID NO: 94 - DNA, 30 bp, Artificial Sequence
  Synthetic
aatctgttacgcacatcatccagacggttg

SEQ ID NO: 95 - DNA, 30 bp, Artificial Sequence
  Synthetic
gaatctgttacgcacatcatccagacggtt

SEQ ID NO: 96 - DNA, 30 bp, Artificial Sequence
  Synthetic
tggcggcagtttgaatctgttacgcacatc

SEQ ID NO: 97 - DNA, 30 bp, Artificial Sequence
  Synthetic
cctgagttgtggcggcagtttgaatctgtt

SEQ ID NO: 98 - DNA, 30 bp, Artificial Sequence
  Synthetic
gcctcagctcggggcccacatgatcatggt

SEQ ID NO: 99 - DNA, 30 bp, Artificial Sequence
  Synthetic
cgcctcagctcggggcccacatgatcatgg

SEQ ID NO: 100 - DNA, 30 bp, Artificial Sequence
  Synthetic
aaactgccgccacaactcaggtaaccatga

SEQ ID NO: 101 - DNA, 30 bp, Artificial Sequence
  Synthetic
tgacagtggacacaccttacctgggcaacc

SEQ ID NO: 102 - DNA, 30 bp, Artificial Sequence
  Synthetic
atcatcccctttctttctcagcctgtcagt

SEQ ID NO: 103 - DNA, 30 bp, Artificial Sequence
  Synthetic
gctgcaactgtatatctacaaggaccgaga

SEQ ID NO: 104 - DNA, 30 bp, Artificial Sequence
  Synthetic
aagaagtggcggaagctggtcctgaggcac

SEQ ID NO: 105 - DNA, 30 bp, Artificial Sequence
  Synthetic
ctgggccacctcctcaattgaagaagtggc

SEQ ID NO: 106 - DNA, 30 bp, Artificial Sequence
  Synthetic
agttcctgggccacctcctcaattgaagaa

SEQ ID NO: 107 - DNA, 30 bp, Artificial Sequence
  Synthetic
tcagcctgtcagtccctgggaacgggcatg

SEQ ID NO: 108 - DNA, 30 bp, Artificial Sequence
  Synthetic
ctcagcctgtcagtccctgggaacgggcat

SEQ ID NO: 109 - DNA, 30 bp, Artificial Sequence
  Synthetic
tttctcagcctgtcagtccctgggaacggg

SEQ ID NO: 110 - DNA, 30 bp, Artificial Sequence
  Synthetic
ctttctcagcctgtcagtccctgggaacgg

SEQ ID NO: 111 - DNA, 30 bp, Artificial Sequence
  Synthetic
cgcacatcatccagacggttgcccaggtaa

SEQ ID NO: 112 - DNA, 30 bp, Artificial Sequence
  Synthetic
gtgacagtggacacaccttacctgggcaac

SEQ ID NO: 113 - DNA, 30 bp, Artificial Sequence
  Synthetic
cccaggtaaggtgtgtccactgtcacaaat

SEQ ID NO: 114 - DNA, 30 bp, Artificial Sequence
  Synthetic
gttggctgcaactgtatatctacaaggacc

SEQ ID NO: 115 - DNA, 30 bp, Artificial Sequence
  Synthetic
tctgcctgccgcactagcttcttggtgact

SEQ ID NO: 116 - DNA, 30 bp, Artificial Sequence
  Synthetic
tagcccatcttctctgcctgccgcactagc

SEQ ID NO: 117 - DNA, 30 bp, Artificial Sequence
  Synthetic
ccagggactgacaggctgagaaagaaaggg

SEQ ID NO: 118 - DNA, 30 bp, Artificial Sequence
  Synthetic
aggaggtggcccaggaactcaacatcatgc

SEQ ID NO: 119 - DNA, 30 bp, Artificial Sequence
  Synthetic
ttcaattgaggaggtggcccaggaactcaa

SEQ ID NO: 120 - DNA, 30 bp, Artificial Sequence
  Synthetic
cgccacttcttcaattgaggaggtggccca

SEQ ID NO: 121 - DNA, 30 bp, Artificial Sequence
  Synthetic
aattgaggaggtggcccaggaactcaacat

SEQ ID NO: 122 - DNA, 30 bp, Artificial Sequence
  Synthetic
tagatatacagttgcagccaacgaagtgcc

SEQ ID NO: 123 - DNA, 30 bp, Artificial Sequence
  Synthetic
tcggtccttgtagatatacagttgcagcca

SEQ ID NO: 124 - DNA, 30 bp, Artificial Sequence
  Synthetic
gtgacttctcggtccttgtagatatacagt

SEQ ID NO: 125 - DNA, 30 bp, Artificial Sequence
  Synthetic
ttggtgacttctcggtccttgtagatatac

SEQ ID NO: 126 - DNA, 30 bp, Artificial Sequence
  Synthetic
cagccaacgaagtgcctcaggaccagcttc

SEQ ID NO: 127 - DNA, 30 bp, Artificial Sequence
  Synthetic
ctaatttggcaaatttctcattttatgcat

SEQ ID NO: 128 - DNA, 30 bp, Artificial Sequence
  Synthetic
atcctaaaataagaaatgcataaaatgaga

SEQ ID NO: 129 - DNA, 30 bp, Artificial Sequence
  Synthetic
aagtagagaaataaacgaacctctcaaaat

SEQ ID NO: 130 - DNA, 30 bp, Artificial Sequence
  Synthetic
tctacttgaattcatactgactttgtgatc

SEQ ID NO: 131 - DNA, 30 bp, Artificial Sequence
  Synthetic
taatttggcaaatttctcattttatgcatt

SEQ ID NO: 132 - DNA, 30 bp, Artificial Sequence
  Synthetic
tatgcatttcttattttaggatgaaaaatt

SEQ ID NO: 133 - DNA, 30 bp, Artificial Sequence
  Synthetic
gcaaatttctcattttatgcatttcttatt

SEQ ID NO: 134 - DNA, 30 bp, Artificial Sequence
  Synthetic
ctcattttatgcatttcttattttaggatg

SEQ ID NO: 135 - DNA, 30 bp, Artificial Sequence
  Synthetic
tcattttatgcatttcttattttaggatga

SEQ ID NO: 136 - DNA, 30 bp, Artificial Sequence
  Synthetic
catcctaaaataagaaatgcataaaatgag

SEQ ID NO: 137 - DNA, 30 bp, Artificial Sequence
  Synthetic
ggcaaatttctcattttatgcatttcttat

SEQ ID NO: 138 - DNA, 30 bp, Artificial Sequence
  Synthetic
tcatcctaaaataagaaatgcataaaatga

SEQ ID NO: 139 - DNA, 30 bp, Artificial Sequence
  Synthetic
tcctcaggagaaaatgataaagtactggtt

SEQ ID NO: 140 - DNA, 30 bp, Artificial Sequence
  Synthetic
aaaatttttcatcctaaaataagaaatgca

SEQ ID NO: 141 - DNA, 30 bp, Artificial Sequence
  Synthetic
caaaatttttcatcctaaaataagaaatgc

SEQ ID NO: 142 - DNA, 30 bp, Artificial Sequence
  Synthetic
ctcaggagaaaatgataaagtactggtttc

SEQ ID NO: 143 - DNA, 30 bp, Artificial Sequence
  Synthetic
cctcaggagaaaatgataaagtactggttt

SEQ ID NO: 144 - DNA, 30 bp, Artificial Sequence
  Synthetic
atgcatttcttattttaggatgaaaaattt

SEQ ID NO: 145 - DNA, 30 bp, Artificial Sequence
  Synthetic
gccacatatgcagcaagtccactgtcgtct

SEQ ID NO: 146 - DNA, 30 bp, Artificial Sequence
  Synthetic
agccacatatgcagcaagtccactgtcgtc

SEQ ID NO: 147 - DNA, 30 bp, Artificial Sequence
  Synthetic
ctttagccacatatgcagcaagtccactgt

SEQ ID NO: 148 - DNA, 30 bp, Artificial Sequence
  Synthetic
ccagctgatagatgggtctattgctttagc

SEQ ID NO: 149 - DNA, 30 bp, Artificial Sequence
  Synthetic
atatcttcccagctgatagatgggtctatt

SEQ ID NO: 150 - DNA, 30 bp, Artificial Sequence
  Synthetic
gatatcttcccagctgatagatgggtctat

SEQ ID NO: 151 - DNA, 30 bp, Artificial Sequence
  Synthetic
tcagccatttgatatcttcccagctgatag

SEQ ID NO: 152 - DNA, 30 bp, Artificial Sequence
  Synthetic
gcaatgatgtcagtcttctcagccatttga

SEQ ID NO: 153 - DNA, 30 bp, Artificial Sequence
  Synthetic
ttcatcctaaaataagaaatgcataaaatg

SEQ ID NO: 154 - DNA, 30 bp, Artificial Sequence
  Synthetic
tgcatttcttattttaggatgaaaaatttt

SEQ ID NO: 155 - DNA, 30 bp, Artificial Sequence
  Synthetic
ggatgaaaaattttgaaaccagtactttat

SEQ ID NO: 156 - DNA, 30 bp, Artificial Sequence
  Synthetic
ttattttaggatgaaaaattttgaaaccag

SEQ ID NO: 157 - DNA, 30 bp, Artificial Sequence
  Synthetic
ccaattgttgcaaagggcattttgagaggt

SEQ ID NO: 158 - DNA, 30 bp, Artificial Sequence
  Synthetic
ttgcaaagggcattttgagaggttcgttta

SEQ ID NO: 159 - DNA, 30 bp, Artificial Sequence
  Synthetic
gcaacaattggcaatgatgtcagtcttctc

SEQ ID NO: 160 - DNA, 30 bp, Artificial Sequence
  Synthetic
caaagggcattttgagaggttcgtttattt

SEQ ID NO: 161 - DNA, 30 bp, Artificial Sequence
  Synthetic
tgagaggttcgtttatttctctacttgaat

SEQ ID NO: 162 - DNA, 30 bp, Artificial Sequence
  Synthetic
gagaggttcgtttatttctctacttgaatt

SEQ ID NO: 163 - DNA, 30 bp, Artificial Sequence
  Synthetic
agaggttcgtttatttctctacttgaattc

SEQ ID NO: 164 - DNA, 30 bp, Artificial Sequence
  Synthetic
gtttatttctctacttgaattcatactgac

SEQ ID NO: 165 - DNA, 30 bp, Artificial Sequence
  Synthetic
atttctctacttgaattcatactgactttg

SEQ ID NO: 166 - DNA, 30 bp, Artificial Sequence
  Synthetic
tttctctacttgaattcatactgactttgt

SEQ ID NO: 167 - DNA, 30 bp, Artificial Sequence
  Synthetic
ctctacttgaattcatactgactttgtgat

SEQ ID NO: 168 - DNA, 30 bp, Artificial Sequence
  Synthetic
ctgcatatgtggctaaagcaatagacccat

SEQ ID NO: 169 - DNA, 30 bp, Artificial Sequence
  Synthetic
cttattttaggatgaaaaattttgaaacca

SEQ ID NO: 170 - DNA, 30 bp, Artificial Sequence
  Synthetic
gagacgacagtggacttgctgcatatgtgg

SEQ ID NO: 171 - DNA, 30 bp, Artificial Sequence
  Synthetic
tggagacgacagtggacttgctgcatatgt

SEQ ID NO: 172 - DNA, 30 bp, Artificial Sequence
  Synthetic
tcctgaggaaaattttggagacgacagtgg

SEQ ID NO: 173 - DNA, 30 bp, Artificial Sequence
  Synthetic
ctcctgaggaaaattttggagacgacagtg

SEQ ID NO: 174 - DNA, 30 bp, Artificial Sequence
  Synthetic
tctcctgaggaaaattttggagacgacagt

SEQ ID NO: 175 - DNA, 30 bp, Artificial Sequence
  Synthetic
tcattttctcctgaggaaaattttggagac

SEQ ID NO: 176 - DNA, 30 bp, Artificial Sequence
  Synthetic
atcattttctcctgaggaaaattttggaga

SEQ ID NO: 177 - DNA, 30 bp, Artificial Sequence
  Synthetic
aaaccagtactttatcattttctcctgagg

SEQ ID NO: 178 - DNA, 30 bp, Artificial Sequence
  Synthetic
gaaaccagtactttatcattttctcctgag

SEQ ID NO: 179 - DNA, 30 bp, Artificial Sequence
  Synthetic
tgaaaccagtactttatcattttctcctga

SEQ ID NO: 180 - DNA, 30 bp, Artificial Sequence
  Synthetic
aggatgaaaaattttgaaaccagtacttta

SEQ ID NO: 181 - DNA, 30 bp, Artificial Sequence
  Synthetic
taggatgaaaaattttgaaaccagtacttt

SEQ ID NO: 182 - DNA, 30 bp, Artificial Sequence
  Synthetic
ttttaggatgaaaaattttgaaaccagtac

SEQ ID NO: 183 - DNA, 30 bp, Artificial Sequence
  Synthetic
ggagacgacagtggacttgctgcatatgtg

SEQ ID NO: 184 - DNA, 30 bp, Artificial Sequence
  Synthetic
caacaattggcaatgatgtcagtcttctca

SEQ ID NO: 185 - DNA, 29 bp, Artificial Sequence
  Synthetic
aattcatactgactttgtgatcctttgtg

SEQ ID NO: 186 - DNA, 24 bp, Artificial Sequence
  Synthetic
atactgactttgtgatcctttgtg

SEQ ID NO: 187 - DNA, 30 bp, Artificial Sequence
  Synthetic
agttacagtttccctaaggtgcttgtttta

SEQ ID NO: 188 - DNA, 30 bp, Artificial Sequence
  Synthetic
aagccatgtttaacagcctccctggcatca

SEQ ID NO: 189 - DNA, 30 bp, Artificial Sequence
  Synthetic
acagcctccctggcatcatcacctggagag

SEQ ID NO: 190 - DNA, 30 bp, Artificial Sequence
  Synthetic
aacagcctccctggcatcatcacctggaga

SEQ ID NO: 191 - DNA, 30 bp, Artificial Sequence
  Synthetic
gacaccaagatcccattcaagccatgttta

SEQ ID NO: 192 - DNA, 30 bp, Artificial Sequence
  Synthetic
tcgagccccatgattcgacaccaagatccc

SEQ ID NO: 193 - DNA, 30 bp, Artificial Sequence
  Synthetic
gcgtctgccaaaactcacagtggctggcac

SEQ ID NO: 194 - DNA, 30 bp, Artificial Sequence
  Synthetic
gcagacgctaagatttccttttggagttcc

SEQ ID NO: 195 - DNA, 30 bp, Artificial Sequence
  Synthetic
tggcagacgctaagatttccttttggagtt

SEQ ID NO: 196 - DNA, 30 bp, Artificial Sequence
  Synthetic
gtgtcgaatcatggggctcgacaactcgat

SEQ ID NO: 197 - DNA, 30 bp, Artificial Sequence
  Synthetic
ggcagacgctaagatttccttttggagttc

SEQ ID NO: 198 - DNA, 30 bp, Artificial Sequence
  Synthetic
aacatggcttgaatgggatcttggtgtcga

SEQ ID NO: 199 - DNA, 30 bp, Artificial Sequence
  Synthetic
ctctctccaggtgatgatgccagggaggct

SEQ ID NO: 200 - DNA, 30 bp, Artificial Sequence
  Synthetic
actctctccaggtgatgatgccagggaggc

SEQ ID NO: 201 - DNA, 30 bp, Artificial Sequence
  Synthetic
tactctctccaggtgatgatgccagggagg

SEQ ID NO: 202 - DNA, 30 bp, Artificial Sequence
  Synthetic
ttttactctctccaggtgatgatgccaggg

SEQ ID NO: 203 - DNA, 30 bp, Artificial Sequence
  Synthetic
cctaaggtgcttgttttactctctccaggt

SEQ ID NO: 204 - DNA, 30 bp, Artificial Sequence
  Synthetic
ccctaaggtgcttgttttactctctccagg

SEQ ID NO: 205 - DNA, 30 bp, Artificial Sequence
  Synthetic
cagtttccctaaggtgcttgttttactctc

SEQ ID NO: 206 - DNA, 30 bp, Artificial Sequence
  Synthetic
aatgggatcttggtgtcgaatcatggggct

SEQ ID NO: 207 - DNA, 24 bp, Artificial Sequence
  Synthetic
ccttttggagttcccatttccatc

SEQ ID NO: 208 - DNA, 23 bp, Artificial Sequence
  Synthetic
cttttggagttcccatttccatc

SEQ ID NO: 209 - DNA, 24 bp, Artificial Sequence
  Synthetic
gggaaactgtaacttaacaggcag

SEQ ID NO: 210 - DNA, 30 bp, Artificial Sequence
  Synthetic
caactttcttttcttttatgatctttaagt

SEQ ID NO: 211 - DNA, 30 bp, Artificial Sequence
  Synthetic
cggttggccatggctctgagtggtaagact

SEQ ID NO: 212 - DNA, 30 bp, Artificial Sequence
  Synthetic
gccatggctctgagtggtaagactcattct

SEQ ID NO: 213 - DNA, 30 bp, Artificial Sequence
  Synthetic
ttgtttacaactttcttttcttttatgatc

SEQ ID NO: 214 - DNA, 30 bp, Artificial Sequence
  Synthetic
tttacaactttcttttcttttatgatcttt

SEQ ID NO: 215 - DNA, 30 bp, Artificial Sequence
  Synthetic
acaactttcttttcttttatgatctttaag

SEQ ID NO: 216 - DNA, 30 bp, Artificial Sequence
  Synthetic
aagatcataaaagaaaagaaagttgtaaac

SEQ ID NO: 217 - DNA, 30 bp, Artificial Sequence
  Synthetic
tctattttatatattcatttctttgtccag

SEQ ID NO: 218 - DNA, 30 bp, Artificial Sequence
  Synthetic
ccactcagagccatggccaaccggaattct

SEQ ID NO: 219 - DNA, 30 bp, Artificial Sequence
  Synthetic
ttcctttagtatctcgaggacatcttgaac

SEQ ID NO: 220 - DNA, 30 bp, Artificial Sequence
  Synthetic
ctttagtatctcgaggacatcttgaacacc

SEQ ID NO: 221 - DNA, 30 bp, Artificial Sequence
  Synthetic
agtatctcgaggacatcttgaacacctttc

SEQ ID NO: 222 - DNA, 30 bp, Artificial Sequence
  Synthetic
gtatctcgaggacatcttgaacacctttct

SEQ ID NO: 223 - DNA, 30 bp, Artificial Sequence
  Synthetic
aagatgtcctcgagatactaaaggaagaat

SEQ ID NO: 224 - DNA, 30 bp, Artificial Sequence
  Synthetic
taaacaagaatgagtcttaccactcagagc

SEQ ID NO: 225 - DNA, 30 bp, Artificial Sequence
  Synthetic
gggggagaaaggtgttcaagatgtcctcga

SEQ ID NO: 226 - DNA, 30 bp, Artificial Sequence
  Synthetic
ccaggtaactggacaaagaaatgaatatat

SEQ ID NO: 227 - DNA, 30 bp, Artificial Sequence
  Synthetic
acttggttagggggagaaaggtgttcaaga

SEQ ID NO: 228 - DNA, 30 bp, Artificial Sequence
  Synthetic
cacttggttagggggagaaaggtgttcaag

SEQ ID NO: 229 - DNA, 30 bp, Artificial Sequence
  Synthetic
tcacttggttagggggagaaaggtgttcaa

SEQ ID NO: 230 - DNA, 30 bp, Artificial Sequence
  Synthetic
tgaatcactctgtatcttttcacttggtta

SEQ ID NO: 231 - DNA, 30 bp, Artificial Sequence
  Synthetic
gttctgaatcactctgtatcttttcacttg

SEQ ID NO: 232 - DNA, 30 bp, Artificial Sequence
  Synthetic
agttctgaatcactctgtatcttttcactt

SEQ ID NO: 233 - DNA, 30 bp, Artificial Sequence
  Synthetic
acagtaaaacaaatgaataaaacaagtcag

SEQ ID NO: 234 - DNA, 30 bp, Artificial Sequence
  Synthetic
caggtaactggacaaagaaatgaatatata

SEQ ID NO: 235 - DNA, 30 bp, Artificial Sequence
  Synthetic
aacacctttctccccctaaccaagtgaaaa

SEQ ID NO: 236 - DNA, 30 bp, Artificial Sequence
  Synthetic
gctttccaggtaactggacaaagaaatgaa

SEQ ID NO: 237 - DNA, 30 bp, Artificial Sequence
  Synthetic
gggcttagctttccaggtaactggacaaag

SEQ ID NO: 238 - DNA, 30 bp, Artificial Sequence
  Synthetic
ggggcttagctttccaggtaactggacaaa

SEQ ID NO: 239 - DNA, 30 bp, Artificial Sequence
  Synthetic
tggggagaccaatcgtttggggcttagctt

SEQ ID NO: 240 - DNA, 30 bp, Artificial Sequence
  Synthetic
gtggggagaccaatcgtttggggcttagct

SEQ ID NO: 241 - DNA, 30 bp, Artificial Sequence
  Synthetic
gttagggggagaaaggtgttcaagatgtcc

SEQ ID NO: 242 - DNA, 30 bp, Artificial Sequence
  Synthetic
ctccccctaaccaagtgaaaagatacagag

SEQ ID NO: 243 - DNA, 30 bp, Artificial Sequence
  Synthetic
tactgtcaagttgtctattttatatattca

SEQ ID NO: 244 - DNA, 30 bp, Artificial Sequence
  Synthetic
agaactaaatcagtctgacttgttttattc

SEQ ID NO: 245 - DNA, 30 bp, Artificial Sequence
  Synthetic
aataatgtgactctattaacactgaattgt

SEQ ID NO: 246 - DNA, 30 bp, Artificial Sequence
  Synthetic
tggcagaacatcaatctggggaaagaaaag

SEQ ID NO: 247 - DNA, 30 bp, Artificial Sequence
  Synthetic
ctggcagaacatcaatctggggaaagaaaa

SEQ ID NO: 248 - DNA, 30 bp, Artificial Sequence
  Synthetic
cacagcctccacaatttctggcagaacatc

SEQ ID NO: 249 - DNA, 30 bp, Artificial Sequence
  Synthetic
ccttccacagcctccacaatttctggcaga

SEQ ID NO: 250 - DNA, 30 bp, Artificial Sequence
  Synthetic
caccttcccttccacagcctccacaatttc

SEQ ID NO: 251 - DNA, 30 bp, Artificial Sequence
  Synthetic
cgcacacccccgtccaggaagacttccacc

SEQ ID NO: 252 - DNA, 30 bp, Artificial Sequence
  Synthetic
ccgcacacccccgtccaggaagacttccac

SEQ ID NO: 253 - DNA, 30 bp, Artificial Sequence
  Synthetic
agaacatcagtgcctttccgcacacccccg

SEQ ID NO: 254 - DNA, 30 bp, Artificial Sequence
  Synthetic
cagaacatcagtgcctttccgcacaccccc

SEQ ID NO: 255 - DNA, 30 bp, Artificial Sequence
  Synthetic
gcgccaagagccagagctttcagaacatca

SEQ ID NO: 256 - DNA, 30 bp, Artificial Sequence
  Synthetic
gtctccccacaaacacagccttggcgccaa

SEQ ID NO: 257 - DNA, 30 bp, Artificial Sequence
  Synthetic
cctggaaagctaagccccaaacgattggtc

SEQ ID NO: 258 - DNA, 30 bp, Artificial Sequence
  Synthetic
tccagttacctggaaagctaagccccaaac

SEQ ID NO: 259 - DNA, 30 bp, Artificial Sequence
  Synthetic
gtccagttacctggaaagctaagccccaaa

SEQ ID NO: 260 - DNA, 30 bp, Artificial Sequence
  Synthetic
tttgtccagttacctggaaagctaagcccc

SEQ ID NO: 261 - DNA, 30 bp, Artificial Sequence
  Synthetic
ctttgtccagttacctggaaagctaagccc

SEQ ID NO: 262 - DNA, 30 bp, Artificial Sequence
  Synthetic
ttttattcatttgttttactgtcaagttgt

SEQ ID NO: 263 - DNA, 30 bp, Artificial Sequence
  Synthetic
tattcatttgttttactgtcaagttgtcta

SEQ ID NO: 264 - DNA, 30 bp, Artificial Sequence
  Synthetic
attcatttgttttactgtcaagttgtctat

SEQ ID NO: 265 - DNA, 30 bp, Artificial Sequence
  Synthetic
ttcatttgttttactgtcaagttgtctatt

SEQ ID NO: 266 - DNA, 30 bp, Artificial Sequence
  Synthetic
atttgttttactgtcaagttgtctatttta

SEQ ID NO: 267 - DNA, 30 bp, Artificial Sequence
  Synthetic
gttttactgtcaagttgtctattttatata

SEQ ID NO: 268 - DNA, 30 bp, Artificial Sequence
  Synthetic
tccccctaaccaagtgaaaagatacagagt

SEQ ID NO: 269 - DNA, 30 bp, Artificial Sequence
  Synthetic
ttttactgtcaagttgtctattttatatat

SEQ ID NO: 270 - DNA, 30 bp, Artificial Sequence
  Synthetic
actgtcaagttgtctattttatatattcat

SEQ ID NO: 271 - DNA, 30 bp, Artificial Sequence
  Synthetic
ctgtcaagttgtctattttatatattcatt

SEQ ID NO: 272 - DNA, 30 bp, Artificial Sequence
  Synthetic
tatatattcatttctttgtccagttacctg

SEQ ID NO: 273 - DNA, 30 bp, Artificial Sequence
  Synthetic
atatattcatttctttgtccagttacctgg

SEQ ID NO: 274 - DNA, 30 bp, Artificial Sequence
  Synthetic
tatattcatttctttgtccagttacctgga

SEQ ID NO: 275 - DNA, 30 bp, Artificial Sequence
  Synthetic
atttctttgtccagttacctggaaagctaa

SEQ ID NO: 276 - DNA, 30 bp, Artificial Sequence
  Synthetic
gcgccaaggctgtgtttgtggggagaccaa

SEQ ID NO: 277 - DNA, 30 bp, Artificial Sequence
  Synthetic
tgaaagctctggctcttggcgccaaggctg

SEQ ID NO: 278 - DNA, 30 bp, Artificial Sequence
  Synthetic
tggaggctgtggaagggaaggtggaagtct

SEQ ID NO: 279 - DNA, 30 bp, Artificial Sequence
  Synthetic
ttgaacttttctttccccagattgatgttc

SEQ ID NO: 280 - DNA, 30 bp, Artificial Sequence
  Synthetic
tgccagaaattgtggaggctgtggaaggga

SEQ ID NO: 281 - DNA, 30 bp, Artificial Sequence
  Synthetic
atgttctgccagaaattgtggaggctgtgg

SEQ ID NO: 282 - DNA, 30 bp, Artificial Sequence
  Synthetic
cccagattgatgttctgccagaaattgtgg

SEQ ID NO: 283 - DNA, 30 bp, Artificial Sequence
  Synthetic
ccccagattgatgttctgccagaaattgtg

SEQ ID NO: 284 - DNA, 30 bp, Artificial Sequence
  Synthetic
tttccccagattgatgttctgccagaaatt

SEQ ID NO: 285 - DNA, 30 bp, Artificial Sequence
  Synthetic
ctttccccagattgatgttctgccagaaat

SEQ ID NO: 286 - DNA, 30 bp, Artificial Sequence
  Synthetic
tctttccccagattgatgttctgccagaaa

SEQ ID NO: 287 - DNA, 30 bp, Artificial Sequence
  Synthetic
aacttttctttccccagattgatgttctgc

SEQ ID NO: 288 - DNA, 30 bp, Artificial Sequence
  Synthetic
atagagtcacattattgaacttttctttcc

SEQ ID NO: 289 - DNA, 30 bp, Artificial Sequence
  Synthetic
agtgttaatagagtcacattattgaacttt

SEQ ID NO: 290 - DNA, 30 bp, Artificial Sequence
  Synthetic
ctggacgggggtgtgcggaaaggcactgat

SEQ ID NO: 291 - DNA, 23 bp, Artificial Sequence
  Synthetic
cttttcttttatgatctttaagt

SEQ ID NO: 292 - DNA, 22 bp, Artificial Sequence
  Synthetic
ttttcttttatgatctttaagt

SEQ ID NO: 293 - DNA, 30 bp, Artificial Sequence
  Synthetic
tttcagggtgccagaatgtgaaagtcatcg

SEQ ID NO: 294 - DNA, 30 bp, Artificial Sequence
  Synthetic
ttttttcagggtgccagaatgtgaaagtca

SEQ ID NO: 295 - DNA, 30 bp, Artificial Sequence
  Synthetic
taagctcaggttcaaagtgttggtaatgcc

SEQ ID NO: 296 - DNA, 30 bp, Artificial Sequence
  Synthetic
atattaaatgtatgcattattttttcaggg

SEQ ID NO: 297 - DNA, 30 bp, Artificial Sequence
  Synthetic
agttcatattaaatgtatgcattatttttt

SEQ ID NO: 298 - DNA, 30 bp, Artificial Sequence
  Synthetic
ttcagggtgccagaatgtgaaagtcatcga

SEQ ID NO: 299 - DNA, 30 bp, Artificial Sequence
  Synthetic
aatgtatgcattattttttcagggtgccag

SEQ ID NO: 300 - DNA, 30 bp, Artificial Sequence
  Synthetic
tcagggtgccagaatgtgaaagtcatcgac

SEQ ID NO: 301 - DNA, 30 bp, Artificial Sequence
  Synthetic
gccgtttccaagatctgacagtgcacaata

SEQ ID NO: 302 - DNA, 30 bp, Artificial Sequence
  Synthetic
agggtgccagaatgtgaaagtcatcgacaa

SEQ ID NO: 303 - DNA, 30 bp, Artificial Sequence
  Synthetic
gtgaggaaaaatcctttggccgtttccaag

SEQ ID NO: 304 - DNA, 30 bp, Artificial Sequence
  Synthetic
ggccgtttccaagatctgacagtgcacaat

SEQ ID NO: 305 - DNA, 30 bp, Artificial Sequence
  Synthetic
cattcagttcatattaaatgtatgcattat

SEQ ID NO: 306 - DNA, 30 bp, Artificial Sequence
  Synthetic
ccaagatctgacagtgcacaatattttccc

SEQ ID NO: 307 - DNA, 30 bp, Artificial Sequence
  Synthetic
caagatctgacagtgcacaatattttccca

SEQ ID NO: 308 - DNA, 30 bp, Artificial Sequence
  Synthetic
cagggtgccagaatgtgaaagtcatcgaca

SEQ ID NO: 309 - DNA, 30 bp, Artificial Sequence
  Synthetic
ttgcattcagttcatattaaatgtatgcat

SEQ ID NO: 310 - DNA, 30 bp, Artificial Sequence
  Synthetic
gaggtagcaaacactaaggtgaaaagataa

SEQ ID NO: 311 - DNA, 30 bp, Artificial Sequence
  Synthetic
agacaacgtcatcccctggcaggctaaagt

SEQ ID NO: 312 - DNA, 30 bp, Artificial Sequence
  Synthetic
aattgtaagctcaggttcaaagtgttggta

SEQ ID NO: 313 - DNA, 30 bp, Artificial Sequence
  Synthetic
ttaaattgtaagctcaggttcaaagtgttg

SEQ ID NO: 314 - DNA, 30 bp, Artificial Sequence
  Synthetic
aaacagtggttcttaaattgtaagctcagg

SEQ ID NO: 315 - DNA, 30 bp, Artificial Sequence
  Synthetic
aaaacagtggttcttaaattgtaagctcag

SEQ ID NO: 316 - DNA, 30 bp, Artificial Sequence
  Synthetic
catgtctttaaaacagtggttcttaaattg

SEQ ID NO: 317 - DNA, 30 bp, Artificial Sequence
  Synthetic
acatgtctttaaaacagtggttcttaaatt

SEQ ID NO: 318 - DNA, 30 bp, Artificial Sequence
  Synthetic
tgtttacatgtctttaaaacagtggttctt

SEQ ID NO: 319 - DNA, 30 bp, Artificial Sequence
  Synthetic
acctgtattctgtttacatgtctttaaaac

SEQ ID NO: 320 - DNA, 30 bp, Artificial Sequence
  Synthetic
ttaacctgtattctgtttacatgtctttaa

SEQ ID NO: 321 - DNA, 30 bp, Artificial Sequence
  Synthetic
attaacctgtattctgtttacatgtcttta

SEQ ID NO: 322 - DNA, 30 bp, Artificial Sequence
  Synthetic
tttattaacctgtattctgtttacatgtct

SEQ ID NO: 323 - DNA, 30 bp, Artificial Sequence
  Synthetic
tcccatctgtattattttttttcagcatgt

SEQ ID NO: 324 - DNA, 30 bp, Artificial Sequence
  Synthetic
gtaaaattggaggtagcaaacactaaggtg

SEQ ID NO: 325 - DNA, 30 bp, Artificial Sequence
  Synthetic
agtaaaattggaggtagcaaacactaaggt

SEQ ID NO: 326 - DNA, 30 bp, Artificial Sequence
  Synthetic
gacaacgtcatcccctggcaggctaaagtg

SEQ ID NO: 327 - DNA, 30 bp, Artificial Sequence
  Synthetic
ttattgcattcagttcatattaaatgtatg

SEQ ID NO: 328 - DNA, 30 bp, Artificial Sequence
  Synthetic
cccatctgtattattttttttcagcatgta

SEQ ID NO: 329 - DNA, 30 bp, Artificial Sequence
  Synthetic
ttcagcatgtattacttgacaaagagacac

SEQ ID NO: 330 - DNA, 30 bp, Artificial Sequence
  Synthetic
ttttttttcagcatgtattacttgacaaag

SEQ ID NO: 331 - DNA, 30 bp, Artificial Sequence
  Synthetic
attgcttttgacttttcaatgggtgtccta

SEQ ID NO: 332 - DNA, 30 bp, Artificial Sequence
  Synthetic
cttttgacttttcaatgggtgtcctaggaa

SEQ ID NO: 333 - DNA, 30 bp, Artificial Sequence
  Synthetic
tgacttttcaatgggtgtcctaggaacctt

SEQ ID NO: 334 - DNA, 30 bp, Artificial Sequence
  Synthetic
gacttttcaatgggtgtcctaggaaccttt

SEQ ID NO: 335 - DNA, 30 bp, Artificial Sequence
  Synthetic
acttttcaatgggtgtcctaggaacctttt

SEQ ID NO: 336 - DNA, 30 bp, Artificial Sequence
  Synthetic
tcaatgggtgtcctaggaaccttttagaaa

SEQ ID NO: 337 - DNA, 30 bp, Artificial Sequence
  Synthetic
caatgggtgtcctaggaaccttttagaaag

SEQ ID NO: 338 - DNA, 30 bp, Artificial Sequence
  Synthetic
aatgggtgtcctaggaaccttttagaaaga

SEQ ID NO: 339 - DNA, 30 bp, Artificial Sequence
  Synthetic
tagaaagaaatggactttcatcctggaaat

SEQ ID NO: 340 - DNA, 30 bp, Artificial Sequence
  Synthetic
agaaagaaatggactttcatcctggaaata

SEQ ID NO: 341 - DNA, 30 bp, Artificial Sequence
  Synthetic
gaaagaaatggactttcatcctggaaatat

SEQ ID NO: 342 - DNA, 30 bp, Artificial Sequence
  Synthetic
catcctggaaatatattaactgttaaaaag

SEQ ID NO: 343 - DNA, 30 bp, Artificial Sequence
  Synthetic
atcctggaaatatattaactgttaaaaaga

SEQ ID NO: 344 - DNA, 30 bp, Artificial Sequence
  Synthetic
actgttaaaaagaaaacattgaaaatgtgt

SEQ ID NO: 345 - DNA, 30 bp, Artificial Sequence
  Synthetic
aaaagaaaacattgaaaatgtgtttagaca

SEQ ID NO: 346 - DNA, 30 bp, Artificial Sequence
  Synthetic
cattgcttttgacttttcaatgggtgtcct

SEQ ID NO: 347 - DNA, 30 bp, Artificial Sequence
  Synthetic
ccatctgtattattttttttcagcatgtat

SEQ ID NO: 348 - DNA, 30 bp, Artificial Sequence
  Synthetic
tttcattgcttttgacttttcaatgggtgt

SEQ ID NO: 349 - DNA, 30 bp, Artificial Sequence
  Synthetic
tatttcattgcttttgacttttcaatgggt

SEQ ID NO: 350 - DNA, 30 bp, Artificial Sequence
  Synthetic
aaaatgtgtttagacaacgtcatcccctgg

SEQ ID NO: 351 - DNA, 30 bp, Artificial Sequence
  Synthetic
tttttcagcatgtattacttgacaaagaga

SEQ ID NO: 352 - DNA, 30 bp, Artificial Sequence
  Synthetic
ttttcagcatgtattacttgacaaagagac

SEQ ID NO: 353 - DNA, 30 bp, Artificial Sequence
  Synthetic
tttcagcatgtattacttgacaaagagaca

SEQ ID NO: 354 - DNA, 30 bp, Artificial Sequence
  Synthetic
tcagcatgtattacttgacaaagagacact

SEQ ID NO: 355 - DNA, 30 bp, Artificial Sequence
  Synthetic
cagcatgtattacttgacaaagagacactg

SEQ ID NO: 356 - DNA, 30 bp, Artificial Sequence
  Synthetic
agcatgtattacttgacaaagagacactgt

SEQ ID NO: 357 - DNA, 30 bp, Artificial Sequence
  Synthetic
cttgacaaagagacactgtgcagagggtga

SEQ ID NO: 358 - DNA, 30 bp, Artificial Sequence
  Synthetic
acaaagagacactgtgcagagggtgaccac

SEQ ID NO: 359 - DNA, 30 bp, Artificial Sequence
  Synthetic
cccacttcaatacaaagggtgtcgttcttt

SEQ ID NO: 360 - DNA, 30 bp, Artificial Sequence
  Synthetic
aatacaaagggtgtcgttcttttccaacaa

SEQ ID NO: 361 - DNA, 30 bp, Artificial Sequence
  Synthetic
ttttccaacaaaatagcaatcccttttatt

SEQ ID NO: 362 - DNA, 30 bp, Artificial Sequence
  Synthetic
tccaacaaaatagcaatcccttttatttca

SEQ ID NO: 363 - DNA, 30 bp, Artificial Sequence
  Synthetic
ccaacaaaatagcaatcccttttatttcat

SEQ ID NO: 364 - DNA, 30 bp, Artificial Sequence
  Synthetic
caacaaaatagcaatcccttttatttcatt

SEQ ID NO: 365 - DNA, 30 bp, Artificial Sequence
  Synthetic
atttcattgcttttgacttttcaatgggtg

SEQ ID NO: 366 - DNA, 30 bp, Artificial Sequence
  Synthetic
aaagtgttggtaatgcctgattcacaactt

SEQ ID NO: 367 - DNA, 30 bp, Artificial Sequence
  Synthetic
ctctctaagaagtaacatacatcctaaaac

SEQ ID NO: 368 - DNA, 30 bp, Artificial Sequence
  Synthetic
acaactttgagaaggtagcactggagagaa

SEQ ID NO: 369 - DNA, 30 bp, Artificial Sequence
  Synthetic
accttagtgtttgctacctccaattttact

SEQ ID NO: 370 - DNA, 30 bp, Artificial Sequence
  Synthetic
gtgtttgctacctccaattttactaaagga

SEQ ID NO: 371 - DNA, 30 bp, Artificial Sequence
  Synthetic
gctacctccaattttactaaaggatacagc

SEQ ID NO: 372 - DNA, 30 bp, Artificial Sequence
  Synthetic
ctacctccaattttactaaaggatacagca

SEQ ID NO: 373 - DNA, 30 bp, Artificial Sequence
  Synthetic
tactaaaggatacagcactttagcctgcca

SEQ ID NO: 374 - DNA, 30 bp, Artificial Sequence
  Synthetic
actaaaggatacagcactttagcctgccag

SEQ ID NO: 375 - DNA, 30 bp, Artificial Sequence
  Synthetic
ctaaaggatacagcactttagcctgccagg

SEQ ID NO: 376 - DNA, 30 bp, Artificial Sequence
  Synthetic
agcctgccaggggatgacgttgtctaaaca

SEQ ID NO: 377 - DNA, 30 bp, Artificial Sequence
  Synthetic
gcctgccaggggatgacgttgtctaaacac

SEQ ID NO: 378 - DNA, 30 bp, Artificial Sequence
  Synthetic
caccttagtgtttgctacctccaattttac

SEQ ID NO: 379 - DNA, 30 bp, Artificial Sequence
  Synthetic
tctaaacacattttcaatgttttcttttta

SEQ ID NO: 380 - DNA, 30 bp, Artificial Sequence
  Synthetic
caatgttttctttttaacagttaatatatt

SEQ ID NO: 381 - DNA, 30 bp, Artificial Sequence
  Synthetic
aatgttttctttttaacagttaatatattt

SEQ ID NO: 382 - DNA, 30 bp, Artificial Sequence
  Synthetic
tctttttaacagttaatatatttccaggat

SEQ ID NO: 383 - DNA, 30 bp, Artificial Sequence
  Synthetic
ctttttaacagttaatatatttccaggatg

SEQ ID NO: 384 - DNA, 30 bp, Artificial Sequence
  Synthetic
tttttaacagttaatatatttccaggatga

SEQ ID NO: 385 - DNA, 30 bp, Artificial Sequence
  Synthetic
ttaacagttaatatatttccaggatgaaag

SEQ ID NO: 386 - DNA, 30 bp, Artificial Sequence
  Synthetic
taacagttaatatatttccaggatgaaagt

SEQ ID NO: 387 - DNA, 30 bp, Artificial Sequence
  Synthetic
aacagttaatatatttccaggatgaaagtc

SEQ ID NO: 388 - DNA, 30 bp, Artificial Sequence
  Synthetic
acagttaatatatttccaggatgaaagtcc

SEQ ID NO: 389 - DNA, 30 bp, Artificial Sequence
  Synthetic
tcaatgttttctttttaacagttaatatat

SEQ ID NO: 390 - DNA, 30 bp, Artificial Sequence
  Synthetic
atatatttccaggatgaaagtccatttctt

SEQ ID NO: 391 - DNA, 30 bp, Artificial Sequence
  Synthetic
tcaccttagtgtttgctacctccaatttta

SEQ ID NO: 392 - DNA, 30 bp, Artificial Sequence
  Synthetic
ataaacaatgagatcattatcttttcacct

SEQ ID NO: 393 - DNA, 30 bp, Artificial Sequence
  Synthetic
tctctaagaagtaacatacatcctaaaaca

SEQ ID NO: 394 - DNA, 30 bp, Artificial Sequence
  Synthetic
ggatatattcagacactaaagatgtgattg

SEQ ID NO: 395 - DNA, 30 bp, Artificial Sequence
  Synthetic
gatatattcagacactaaagatgtgattgg

SEQ ID NO: 396 - DNA, 30 bp, Artificial Sequence
  Synthetic
agacactaaagatgtgattggaaatctaca

SEQ ID NO: 397 - DNA, 30 bp, Artificial Sequence
  Synthetic
gaaatctacattcaaagaagtatcaccaat

SEQ ID NO: 398 - DNA, 30 bp, Artificial Sequence
  Synthetic
aaagaagtatcaccaattaccgccacccat

SEQ ID NO: 399 - DNA, 30 bp, Artificial Sequence
  Synthetic
ccgccacccattccaattctctccagtgct

SEQ ID NO: 400 - DNA, 30 bp, Artificial Sequence
  Synthetic
caattctctccagtgctaccttctcaaagt

SEQ ID NO: 401 - DNA, 30 bp, Artificial Sequence
  Synthetic
tctccagtgctaccttctcaaagttgtgaa

SEQ ID NO: 402 - DNA, 30 bp, Artificial Sequence
  Synthetic
tcttttcaccttagtgtttgctacctccaa

SEQ ID NO: 403 - DNA, 30 bp, Artificial Sequence
  Synthetic
tcaaagttgtgaatcaggcattaccaacac

SEQ ID NO: 404 - DNA, 30 bp, Artificial Sequence
  Synthetic
ccaacactttgaacctgagcttacaattta

SEQ ID NO: 405 - DNA, 30 bp, Artificial Sequence
  Synthetic
gaacctgagcttacaatttaagaaccactg

SEQ ID NO: 406 - DNA, 30 bp, Artificial Sequence
  Synthetic
aacctgagcttacaatttaagaaccactgt

SEQ ID NO: 407 - DNA, 30 bp, Artificial Sequence
  Synthetic
caatttaagaaccactgttttaaagacatg

SEQ ID NO: 408 - DNA, 30 bp, Artificial Sequence
  Synthetic
aagaaccactgttttaaagacatgtaaaca

SEQ ID NO: 409 - DNA, 30 bp, Artificial Sequence
  Synthetic
agaaccactgttttaaagacatgtaaacag

SEQ ID NO: 410 - DNA, 30 bp, Artificial Sequence
  Synthetic
taaagacatgtaaacagaatacaggttaat

SEQ ID NO: 411 - DNA, 30 bp, Artificial Sequence
  Synthetic
aaagacatgtaaacagaatacaggttaata

SEQ ID NO: 412 - DNA, 30 bp, Artificial Sequence
  Synthetic
aagacatgtaaacagaatacaggttaataa

SEQ ID NO: 413 - DNA, 30 bp, Artificial Sequence
  Synthetic
tgaatcaggcattaccaacactttgaacct

SEQ ID NO: 414 - DNA, 30 bp, Artificial Sequence
  Synthetic
gtaatgcctgattcacaactttgagaaggt

SEQ ID NO: 415 - DNA, 30 bp, Artificial Sequence
  Synthetic
ccaggatgaaagtccatttctttctaaaag

SEQ ID NO: 416 - DNA, 30 bp, Artificial Sequence
  Synthetic
atttctctctaagaagtaacatacatccta

SEQ ID NO: 417 - DNA, 30 bp, Artificial Sequence
  Synthetic
acattctggcaccctgaaaaaataatgcat

SEQ ID NO: 418 - DNA, 30 bp, Artificial Sequence
  Synthetic
tggcaccctgaaaaaataatgcatacattt

SEQ ID NO: 419 - DNA, 30 bp, Artificial Sequence
  Synthetic
tttctctctaagaagtaacatacatcctaa

SEQ ID NO: 420 - DNA, 30 bp, Artificial Sequence
  Synthetic
ccaaaaatgctttatttctctctaagaagt

SEQ ID NO: 421 - DNA, 30 bp, Artificial Sequence
  Synthetic
ttagagagaaataaagcatttttgggaaga

SEQ ID NO: 422 - DNA, 30 bp, Artificial Sequence
  Synthetic
cttcttagagagaaataaagcatttttggg

SEQ ID NO: 423 - DNA, 30 bp, Artificial Sequence
  Synthetic
ggatgtatgttacttcttagagagaaataa

SEQ ID NO: 424 - DNA, 30 bp, Artificial Sequence
  Synthetic
aggatgtatgttacttcttagagagaaata

SEQ ID NO: 425 - DNA, 30 bp, Artificial Sequence
  Synthetic
taggatgtatgttacttcttagagagaaat

SEQ ID NO: 426 - DNA, 30 bp, Artificial Sequence
  Synthetic
cacattctggcaccctgaaaaaataatgca

SEQ ID NO: 427 - DNA, 30 bp, Artificial Sequence
  Synthetic
gtgtctgaatatatccaaatgttttaggat

SEQ ID NO: 428 - DNA, 30 bp, Artificial Sequence
  Synthetic
caatcacatctttagtgtctgaatatatcc

SEQ ID NO: 429 - DNA, 30 bp, Artificial Sequence
  Synthetic
ccaatcacatctttagtgtctgaatatatc

SEQ ID NO: 430 - DNA, 30 bp, Artificial Sequence
  Synthetic
aatgtagatttccaatcacatctttagtgt

SEQ ID NO: 431 - DNA, 30 bp, Artificial Sequence
  Synthetic
gaatgtagatttccaatcacatctttagtg

SEQ ID NO: 432 - DNA, 30 bp, Artificial Sequence
  Synthetic
tttgaatgtagatttccaatcacatcttta

SEQ ID NO: 433 - DNA, 30 bp, Artificial Sequence
  Synthetic
gtgatacttctttgaatgtagatttccaat

SEQ ID NO: 434 - DNA, 30 bp, Artificial Sequence
  Synthetic
gaatgggtggcggtaattggtgatacttct

SEQ ID NO: 435 - DNA, 30 bp, Artificial Sequence
  Synthetic
agaaggtagcactggagagaattggaatgg

SEQ ID NO: 436 - DNA, 30 bp, Artificial Sequence
  Synthetic
gagaaggtagcactggagagaattggaatg

SEQ ID NO: 437 - DNA, 30 bp, Artificial Sequence
  Synthetic
agtgtctgaatatatccaaatgttttagga

SEQ ID NO: 438 - DNA, 30 bp, Artificial Sequence
  Synthetic
caggatgaaagtccatttctttctaaaagg

SEQ ID NO: 439 - DNA, 30 bp, Artificial Sequence
  Synthetic
tcgatgactttcacattctggcaccctgaa

SEQ ID NO: 440 - DNA, 30 bp, Artificial Sequence
  Synthetic
cctcaccaatgtcttgtcgatgactttcac

SEQ ID NO: 441 - DNA, 30 bp, Artificial Sequence
  Synthetic
tttctaaaaggttcctaggacacccattga

SEQ ID NO: 442 - DNA, 30 bp, Artificial Sequence
  Synthetic
ctaaaaggttcctaggacacccattgaaaa

SEQ ID NO: 443 - DNA, 30 bp, Artificial Sequence
  Synthetic
taaaaggttcctaggacacccattgaaaag

SEQ ID NO: 444 - DNA, 30 bp, Artificial Sequence
  Synthetic
ctaggacacccattgaaaagtcaaaagcaa

SEQ ID NO: 445 - DNA, 30 bp, Artificial Sequence
  Synthetic
aaaagtcaaaagcaatgaaataaaagggat

SEQ ID NO: 446 - DNA, 30 bp, Artificial Sequence
  Synthetic
ctattttgttggaaaagaacgacacccttt

SEQ ID NO: 447 - DNA, 30 bp, Artificial Sequence
  Synthetic
tgttggaaaagaacgacaccctttgtattg

SEQ ID NO: 448 - DNA, 30 bp, Artificial Sequence
  Synthetic
gttggaaaagaacgacaccctttgtattga

SEQ ID NO: 449 - DNA, 30 bp, Artificial Sequence
  Synthetic
ttggaaaagaacgacaccctttgtattgaa

SEQ ID NO: 450 - DNA, 30 bp, Artificial Sequence
  Synthetic
ctcaccaatgtcttgtcgatgactttcaca

SEQ ID NO: 451 - DNA, 30 bp, Artificial Sequence
  Synthetic
gaaaagaacgacaccctttgtattgaagtg

SEQ ID NO: 452 - DNA, 30 bp, Artificial Sequence
  Synthetic
tattgaagtggggaattacagactgtggtc

SEQ ID NO: 453 - DNA, 30 bp, Artificial Sequence
  Synthetic
aagtggggaattacagactgtggtcaccct

SEQ ID NO: 454 - DNA, 30 bp, Artificial Sequence
  Synthetic
cagactgtggtcaccctctgcacagtgtct

SEQ ID NO: 455 - DNA, 30 bp, Artificial Sequence
  Synthetic
gtcaagtaatacatgctgaaaaaaaataat

SEQ ID NO: 456 - DNA, 30 bp, Artificial Sequence
  Synthetic
tcaagtaatacatgctgaaaaaaaataata

SEQ ID NO: 457 - DNA, 30 bp, Artificial Sequence
  Synthetic
tgcactgtcagatcttggaaacggccaaag

SEQ ID NO: 458 - DNA, 30 bp, Artificial Sequence
  Synthetic
gaaacggccaaaggatttttcctcaccaat

SEQ ID NO: 459 - DNA, 30 bp, Artificial Sequence
  Synthetic
ttcctcaccaatgtcttgtcgatgactttc

SEQ ID NO: 460 - DNA, 30 bp, Artificial Sequence
  Synthetic
tcctcaccaatgtcttgtcgatgactttca

SEQ ID NO: 461 - DNA, 30 bp, Artificial Sequence
  Synthetic
gtattgaagtggggaattacagactgtggt

SEQ ID NO: 462 - DNA, 30 bp, Artificial Sequence
  Synthetic
ctttctaaaaggttcctaggacacccattg

SEQ ID NO: 463 - DNA, 28 bp, Artificial Sequence
  Synthetic
gagagaaataaagcatttttgggaagaa

SEQ ID NO: 464 - DNA, 27 bp, Artificial Sequence
  Synthetic
aatatgaactgaatgcaataataatca

SEQ ID NO: 465 - DNA, 26 bp, Artificial Sequence
  Synthetic
atatgaactgaatgcaataataatca

SEQ ID NO: 466 - RNA, 30 nt, Artificial Sequence
  Synthetic
ccuggaaaaugcugcaauauuaucagccaa

SEQ ID NO: 467 - RNA, 30 nt, Artificial Sequence
  Synthetic
ucuuaccuggaaaaugcugcaauauuauca

SEQ ID NO: 468 - RNA, 30 nt, Artificial Sequence
  Synthetic
cuuaccuggaaaaugcugcaauauuaucag

SEQ ID NO: 469 - RNA, 30 nt, Artificial Sequence
  Synthetic
uuaccuggaaaaugcugcaauauuaucagc

SEQ ID NO: 470 - RNA, 30 nt, Artificial Sequence
  Synthetic
ucagccaaaguuucuucaucauuugcccca

SEQ ID NO: 471 - RNA, 30 nt, Artificial Sequence
  Synthetic
cuucaucauuugccccagaccuguaauagu

SEQ ID NO: 472 - RNA, 30 nt, Artificial Sequence
  Synthetic
uucaucauuugccccagaccuguaauaguc

SEQ ID NO: 473 - RNA, 30 nt, Artificial Sequence
  Synthetic
aucauuugccccagaccuguaauagucaua

SEQ ID NO: 474 - RNA, 30 nt, Artificial Sequence
  Synthetic
gccccagaccuguaauagucauauauagac

SEQ ID NO: 475 - RNA, 30 nt, Artificial Sequence
  Synthetic
ccccagaccuguaauagucauauauagacu

SEQ ID NO: 476 - RNA, 30 nt, Artificial Sequence
  Synthetic
aaaaaauaaauuuucuuaccuggaaaaugc

SEQ ID NO: 477 - RNA, 30 nt, Artificial Sequence
  Synthetic
ggaaguacugauuuagcauguuguucauaa

SEQ ID NO: 478 - RNA, 30 nt, Artificial Sequence
  Synthetic
agcauguuguucauaaucauugauacaaau

SEQ ID NO: 479 - RNA, 30 nt, Artificial Sequence
  Synthetic
gcauguuguucauaaucauugauacaaauu

SEQ ID NO: 480 - RNA, 30 nt, Artificial Sequence
  Synthetic
uucauaaucauugauacaaauuagccgggg

SEQ ID NO: 481 - RNA, 30 nt, Artificial Sequence
  Synthetic
auaaucauugauacaaauuagccgggggag

SEQ ID NO: 482 - RNA, 30 nt, Artificial Sequence
  Synthetic
auacaaauuagccgggggagcauuuucaca

SEQ ID NO: 483 - RNA, 30 nt, Artificial Sequence
  Synthetic
gccgggggagcauuuucacagguuauugcu

SEQ ID NO: 484 - RNA, 30 nt, Artificial Sequence
  Synthetic
ucacagguuauugcuaucccagauggaguu

SEQ ID NO: 485 - RNA, 30 nt, Artificial Sequence
  Synthetic
cacagguuauugcuaucccagauggaguuc

SEQ ID NO: 486 - RNA, 30 nt, Artificial Sequence
  Synthetic
acagguuauugcuaucccagauggaguucg

SEQ ID NO: 487 - RNA, 30 nt, Artificial Sequence
  Synthetic
gaaguacugauuuagcauguuguucauaau

SEQ ID NO: 488 - RNA, 30 nt, Artificial Sequence
  Synthetic
uaaaaaauaaauuuucuuaccuggaaaaug

SEQ ID NO: 489 - RNA, 30 nt, Artificial Sequence
  Synthetic
aaaaauaaauuuucuuaccuggaaaaugcu

SEQ ID NO: 490 - RNA, 30 nt, Artificial Sequence
  Synthetic
aaaacaugauuuuaaaaaauaaauuuucuu

SEQ ID NO: 491 - RNA, 30 nt, Artificial Sequence
  Synthetic
uaucaaugauuaugaacaacaugcuaaauc

SEQ ID NO: 492 - RNA, 30 nt, Artificial Sequence
  Synthetic
ugaacaacaugcuaaaucaguacuuccaaa

SEQ ID NO: 493 - RNA, 30 nt, Artificial Sequence
  Synthetic
caaagucuauauaugacuauuacaggucug

SEQ ID NO: 494 - RNA, 30 nt, Artificial Sequence
  Synthetic
caggucuggggcaaaugaugaagaaacuuu

SEQ ID NO: 495 - RNA, 30 nt, Artificial Sequence
  Synthetic
ggcugauaauauugcagcauuuuccaggua

SEQ ID NO: 496 - RNA, 30 nt, Artificial Sequence
  Synthetic
gcugauaauauugcagcauuuuccagguaa

SEQ ID NO: 497 - RNA, 30 nt, Artificial Sequence
  Synthetic
cagcauuuuccagguaagaaaauuuauuuu

SEQ ID NO: 498 - RNA, 30 nt, Artificial Sequence
  Synthetic
uccagguaagaaaauuuauuuuuuaaaauc

SEQ ID NO: 499 - RNA, 30 nt, Artificial Sequence
  Synthetic
ccagguaagaaaauuuauuuuuuaaaauca

SEQ ID NO: 500 - RNA, 30 nt, Artificial Sequence
  Synthetic
aaacaugauuuuaaaaaauaaauuuucuua

SEQ ID NO: 501 - RNA, 30 nt, Artificial Sequence
  Synthetic
auuuuuuaaaaucauguuuuaaaauuacac

SEQ ID NO: 502 - RNA, 30 nt, Artificial Sequence
  Synthetic
cagguaagaaaauuuauuuuuuaaaaucau

SEQ ID NO: 503 - RNA, 30 nt, Artificial Sequence
  Synthetic
uuuaaaaucauguuuuaaaauuacacaaag

SEQ ID NO: 504 - RNA, 30 nt, Artificial Sequence
  Synthetic
uuaaaaucauguuuuaaaauuacacaaaga

SEQ ID NO: 505 - RNA, 30 nt, Artificial Sequence
  Synthetic
uaaaaucauguuuuaaaauuacacaaagac

SEQ ID NO: 506 - RNA, 30 nt, Artificial Sequence
  Synthetic
aaaaucauguuuuaaaauuacacaaagacc

SEQ ID NO: 507 - RNA, 30 nt, Artificial Sequence
  Synthetic
aaaucauguuuuaaaauuacacaaagaccg

SEQ ID NO: 508 - RNA, 30 nt, Artificial Sequence
  Synthetic
guguaauuuuaaaacaugauuuuaaaaaau

SEQ ID NO: 509 - RNA, 30 nt, Artificial Sequence
  Synthetic
uguaauuuuaaaacaugauuuuaaaaaaua

SEQ ID NO: 510 - RNA, 30 nt, Artificial Sequence
  Synthetic
uaaaacaugauuuuaaaaaauaaauuuucu

SEQ ID NO: 511 - RNA, 30 nt, Artificial Sequence
  Synthetic
uuuuuuaaaaucauguuuuaaaauuacaca

SEQ ID NO: 512 - RNA, 30 nt, Artificial Sequence
  Synthetic
guaucaaugauuaugaacaacaugcuaaau

SEQ ID NO: 513 - RNA, 24 nt, Artificial Sequence
  Synthetic
uugcuaucccagauggaguucguu

SEQ ID NO: 514 - RNA, 21 nt, Artificial Sequence
  Synthetic
cuaucccagauggaguucguu

SEQ ID NO: 515 - RNA, 30 nt, Artificial Sequence
  Synthetic
uuuuuuaauucuagauggaagcuguaucca

SEQ ID NO: 516 - RNA, 30 nt, Artificial Sequence
  Synthetic
auuuuauuuuuuaauucuagauggaagcug

SEQ ID NO: 517 - RNA, 30 nt, Artificial Sequence
  Synthetic
auuuuuuaauucuagauggaagcuguaucc

SEQ ID NO: 518 - RNA, 30 nt, Artificial Sequence
  Synthetic
uauuuuuuaauucuagauggaagcuguauc

SEQ ID NO: 519 - RNA, 30 nt, Artificial Sequence
  Synthetic
uuuuauuuuuuaauucuagauggaagcugu

SEQ ID NO: 520 - RNA, 30 nt, Artificial Sequence
  Synthetic
aaaaauaaaauaaaauaaaaggcuuuagag

SEQ ID NO: 521 - RNA, 30 nt, Artificial Sequence
  Synthetic
auuuuauuuuauuuuuuaauucuagaugga

SEQ ID NO: 522 - RNA, 30 nt, Artificial Sequence
  Synthetic
uauuuuauuuuauuuuuuaauucuagaugg

SEQ ID NO: 523 - RNA, 30 nt, Artificial Sequence
  Synthetic
ugaaacucuaaagccuuuuauuuuauuuua

SEQ ID NO: 524 - RNA, 30 nt, Artificial Sequence
  Synthetic
uuuaauucuagauggaagcuguauccaagg

SEQ ID NO: 525 - RNA, 30 nt, Artificial Sequence
  Synthetic
uuuuauuuuauuuuuuaauucuagauggaa

SEQ ID NO: 526 - RNA, 30 nt, Artificial Sequence
  Synthetic
uuaauucuagauggaagcuguauccaagga

SEQ ID NO: 527 - RNA, 30 nt, Artificial Sequence
  Synthetic
uauuuuauuuuuuaauucuagauggaagcu

SEQ ID NO: 528 - RNA, 30 nt, Artificial Sequence
  Synthetic
caucuagaauuaaaaaauaaaauaaaauaa

SEQ ID NO: 529 - RNA, 30 nt, Artificial Sequence
  Synthetic
uaauucuagauggaagcuguauccaaggau

SEQ ID NO: 530 - RNA, 30 nt, Artificial Sequence
  Synthetic
aauucuagauggaagcuguauccaaggaug

SEQ ID NO: 531 - RNA, 30 nt, Artificial Sequence
  Synthetic
auucuagauggaagcuguauccaaggaugc

SEQ ID NO: 532 - RNA, 30 nt, Artificial Sequence
  Synthetic
uagauggaagcuguauccaaggaugcuccg

SEQ ID NO: 533 - RNA, 30 nt, Artificial Sequence
  Synthetic
cugaaacagaucugucgacuucuguuuuag

SEQ ID NO: 534 - RNA, 30 nt, Artificial Sequence
  Synthetic
uaggacagagggucagcaugccaauaugug

SEQ ID NO: 535 - RNA, 30 nt, Artificial Sequence
  Synthetic
aggacagagggucagcaugccaauaugugu

SEQ ID NO: 536 - RNA, 30 nt, Artificial Sequence
  Synthetic
ggacagagggucagcaugccaauaugugug

SEQ ID NO: 537 - RNA, 30 nt, Artificial Sequence
  Synthetic
ccacugugagagguaggaggaagauuguca

SEQ ID NO: 538 - RNA, 30 nt, Artificial Sequence
  Synthetic
uguuuuaggacagagggucagcaugccaau

SEQ ID NO: 539 - RNA, 30 nt, Artificial Sequence
  Synthetic
gccuccuucugucccuguggugacaaucuu

SEQ ID NO: 540 - RNA, 30 nt, Artificial Sequence
  Synthetic
ucaccacagggacagaaggaggcuaacguu

SEQ ID NO: 541 - RNA, 30 nt, Artificial Sequence
  Synthetic
cggagcauccuuggauacagcuuccaucua

SEQ ID NO: 542 - RNA, 30 nt, Artificial Sequence
  Synthetic
agcaacauuccggagcauccuuggauacag

SEQ ID NO: 543 - RNA, 30 nt, Artificial Sequence
  Synthetic
cagcaacauuccggagcauccuuggauaca

SEQ ID NO: 544 - RNA, 30 nt, Artificial Sequence
  Synthetic
gauacagcuuccaucuagaauuaaaaaaua

SEQ ID NO: 545 - RNA, 30 nt, Artificial Sequence
  Synthetic
cuccuaccucucacaguggcaagcucgccg

SEQ ID NO: 546 - RNA, 30 nt, Artificial Sequence
  Synthetic
ugucccuguggugacaaucuuccuccuacc

SEQ ID NO: 547 - RNA, 30 nt, Artificial Sequence
  Synthetic
gcaugcugacccucuguccuaaaacagaag

SEQ ID NO: 548 - RNA, 30 nt, Artificial Sequence
  Synthetic
ccugggcaaccgucuggaugaugugcguaa

SEQ ID NO: 549 - RNA, 30 nt, Artificial Sequence
  Synthetic
aaucuguuacgcacaucauccagacgguug

SEQ ID NO: 550 - RNA, 30 nt, Artificial Sequence
  Synthetic
gaaucuguuacgcacaucauccagacgguu

SEQ ID NO: 551 - RNA, 30 nt, Artificial Sequence
  Synthetic
uggcggcaguuugaaucuguuacgcacauc

SEQ ID NO: 552 - RNA, 30 nt, Artificial Sequence
  Synthetic
ccugaguuguggcggcaguuugaaucuguu

SEQ ID NO: 553 - RNA, 30 nt, Artificial Sequence
  Synthetic
gccucagcucggggcccacaugaucauggu

SEQ ID NO: 554 - RNA, 30 nt, Artificial Sequence
  Synthetic
cgccucagcucggggcccacaugaucaugg

SEQ ID NO: 555 - RNA, 30 nt, Artificial Sequence
  Synthetic
aaacugccgccacaacucagguaaccauga

SEQ ID NO: 556 - RNA, 30 nt, Artificial Sequence
  Synthetic
ugacaguggacacaccuuaccugggcaacc

SEQ ID NO: 557 - RNA, 30 nt, Artificial Sequence
  Synthetic
aucauccccuuucuuucucagccugucagu

SEQ ID NO: 558 - RNA, 30 nt, Artificial Sequence
  Synthetic
gcugcaacuguauaucuacaaggaccgaga

SEQ ID NO: 559 - RNA, 30 nt, Artificial Sequence
  Synthetic
aagaaguggcggaagcugguccugaggcac

SEQ ID NO: 560 - RNA, 30 nt, Artificial Sequence
  Synthetic
cugggccaccuccucaauugaagaaguggc

SEQ ID NO: 561 - RNA, 30 nt, Artificial Sequence
  Synthetic
aguuccugggccaccuccucaauugaagaa

SEQ ID NO: 562 - RNA, 30 nt, Artificial Sequence
  Synthetic
ucagccugucagucccugggaacgggcaug

SEQ ID NO: 563 - RNA, 30 nt, Artificial Sequence
  Synthetic
cucagccugucagucccugggaacgggcau

SEQ ID NO: 564 - RNA, 30 nt, Artificial Sequence
  Synthetic
uuucucagccugucagucccugggaacggg

SEQ ID NO: 565 - RNA, 30 nt, Artificial Sequence
  Synthetic
cuuucucagccugucagucccugggaacgg

SEQ ID NO: 566 - RNA, 30 nt, Artificial Sequence
  Synthetic
cgcacaucauccagacgguugcccagguaa

SEQ ID NO: 567 - RNA, 30 nt, Artificial Sequence
  Synthetic
gugacaguggacacaccuuaccugggcaac

SEQ ID NO: 568 - RNA, 30 nt, Artificial Sequence
  Synthetic
cccagguaagguguguccacugucacaaau

SEQ ID NO: 569 - RNA, 30 nt, Artificial Sequence
  Synthetic
guuggcugcaacuguauaucuacaaggacc

SEQ ID NO: 570 - RNA, 30 nt, Artificial Sequence
  Synthetic
ucugccugccgcacuagcuucuuggugacu

SEQ ID NO: 571 - RNA, 30 nt, Artificial Sequence
  Synthetic
uagcccaucuucucugccugccgcacuagc

SEQ ID NO: 572 - RNA, 30 nt, Artificial Sequence
  Synthetic
ccagggacugacaggcugagaaagaaaggg

SEQ ID NO: 573 - RNA, 30 nt, Artificial Sequence
  Synthetic
aggagguggcccaggaacucaacaucaugc

SEQ ID NO: 574 - RNA, 30 nt, Artificial Sequence
  Synthetic
uucaauugaggagguggcccaggaacucaa

SEQ ID NO: 575 - RNA, 30 nt, Artificial Sequence
  Synthetic
cgccacuucuucaauugaggagguggccca

SEQ ID NO: 576 - RNA, 30 nt, Artificial Sequence
  Synthetic
aauugaggagguggcccaggaacucaacau

SEQ ID NO: 577 - RNA, 30 nt, Artificial Sequence
  Synthetic
uagauauacaguugcagccaacgaagugcc

SEQ ID NO: 578 - RNA, 30 nt, Artificial Sequence
  Synthetic
ucgguccuuguagauauacaguugcagcca

SEQ ID NO: 579 - RNA, 30 nt, Artificial Sequence
  Synthetic
gugacuucucgguccuuguagauauacagu

SEQ ID NO: 580 - RNA, 30 nt, Artificial Sequence
  Synthetic
uuggugacuucucgguccuuguagauauac

SEQ ID NO: 581 - RNA, 30 nt, Artificial Sequence
  Synthetic
cagccaacgaagugccucaggaccagcuuc

SEQ ID NO: 582 - RNA, 30 nt, Artificial Sequence
  Synthetic
cuaauuuggcaaauuucucauuuuaugcau

SEQ ID NO: 583 - RNA, 30 nt, Artificial Sequence
  Synthetic
auccuaaaauaagaaaugcauaaaaugaga

SEQ ID NO: 584 - RNA, 30 nt, Artificial Sequence
  Synthetic
aaguagagaaauaaacgaaccucucaaaau

SEQ ID NO: 585 - RNA, 30 nt, Artificial Sequence
  Synthetic
ucuacuugaauucauacugacuuugugauc

SEQ ID NO: 586 - RNA, 30 nt, Artificial Sequence
  Synthetic
uaauuuggcaaauuucucauuuuaugcauu

SEQ ID NO: 587 - RNA, 30 nt, Artificial Sequence
  Synthetic
uaugcauuucuuauuuuaggaugaaaaauu

SEQ ID NO: 588 - RNA, 30 nt, Artificial Sequence
  Synthetic
gcaaauuucucauuuuaugcauuucuuauu

SEQ ID NO: 589 - RNA, 30 nt, Artificial Sequence
  Synthetic
cucauuuuaugcauuucuuauuuuaggaug

SEQ ID NO: 590 - RNA, 30 nt, Artificial Sequence
  Synthetic
ucauuuuaugcauuucuuauuuuaggauga

SEQ ID NO: 591 - RNA, 30 nt, Artificial Sequence
  Synthetic
cauccuaaaauaagaaaugcauaaaaugag

SEQ ID NO: 592 - RNA, 30 nt, Artificial Sequence
  Synthetic
ggcaaauuucucauuuuaugcauuucuuau

SEQ ID NO: 593 - RNA, 30 nt, Artificial Sequence
  Synthetic
ucauccuaaaauaagaaaugcauaaaauga

SEQ ID NO: 594 - RNA, 30 nt, Artificial Sequence
  Synthetic
uccucaggagaaaaugauaaaguacugguu

SEQ ID NO: 595 - RNA, 30 nt, Artificial Sequence
  Synthetic
aaaauuuuucauccuaaaauaagaaaugca

SEQ ID NO: 596 - RNA, 30 nt, Artificial Sequence
  Synthetic
caaaauuuuucauccuaaaauaagaaaugc

SEQ ID NO: 597 - RNA, 30 nt, Artificial Sequence
  Synthetic
cucaggagaaaaugauaaaguacugguuuc

SEQ ID NO: 598 - RNA, 30 nt, Artificial Sequence
  Synthetic
ccucaggagaaaaugauaaaguacugguuu

SEQ ID NO: 599 - RNA, 30 nt, Artificial Sequence
  Synthetic
augcauuucuuauuuuaggaugaaaaauuu

SEQ ID NO: 600 - RNA, 30 nt, Artificial Sequence
  Synthetic
gccacauaugcagcaaguccacugucgucu

SEQ ID NO: 601 - RNA, 30 nt, Artificial Sequence
  Synthetic
agccacauaugcagcaaguccacugucguc

SEQ ID NO: 602 - RNA, 30 nt, Artificial Sequence
  Synthetic
cuuuagccacauaugcagcaaguccacugu

SEQ ID NO: 603 - RNA, 30 nt, Artificial Sequence
  Synthetic
ccagcugauagaugggucuauugcuuuagc

SEQ ID NO: 604 - RNA, 30 nt, Artificial Sequence
  Synthetic
auaucuucccagcugauagaugggucuauu

SEQ ID NO: 605 - RNA, 30 nt, Artificial Sequence
  Synthetic
gauaucuucccagcugauagaugggucuau

SEQ ID NO: 606 - RNA, 30 nt, Artificial Sequence
  Synthetic
ucagccauuugauaucuucccagcugauag

SEQ ID NO: 607 - RNA, 30 nt, Artificial Sequence
  Synthetic
gcaaugaugucagucuucucagccauuuga

SEQ ID NO: 608 - RNA, 30 nt, Artificial Sequence
  Synthetic
uucauccuaaaauaagaaaugcauaaaaug

SEQ ID NO: 609 - RNA, 30 nt, Artificial Sequence
  Synthetic
ugcauuucuuauuuuaggaugaaaaauuuu

SEQ ID NO: 610 - RNA, 30 nt, Artificial Sequence
  Synthetic
ggaugaaaaauuuugaaaccaguacuuuau

SEQ ID NO: 611 - RNA, 30 nt, Artificial Sequence
  Synthetic
uuauuuuaggaugaaaaauuuugaaaccag

SEQ ID NO: 612 - RNA, 30 nt, Artificial Sequence
  Synthetic
ccaauuguugcaaagggcauuuugagaggu

SEQ ID NO: 613 - RNA, 30 nt, Artificial Sequence
  Synthetic
uugcaaagggcauuuugagagguucguuua

SEQ ID NO: 614 - RNA, 30 nt, Artificial Sequence
  Synthetic
gcaacaauuggcaaugaugucagucuucuc

SEQ ID NO: 615 - RNA, 30 nt, Artificial Sequence
  Synthetic
caaagggcauuuugagagguucguuuauuu

SEQ ID NO: 616 - RNA, 30 nt, Artificial Sequence
  Synthetic
ugagagguucguuuauuucucuacuugaau

SEQ ID NO: 617 - RNA, 30 nt, Artificial Sequence
  Synthetic
gagagguucguuuauuucucuacuugaauu

SEQ ID NO: 618 - RNA, 30 nt, Artificial Sequence
  Synthetic
agagguucguuuauuucucuacuugaauuc

SEQ ID NO: 619 - RNA, 30 nt, Artificial Sequence
  Synthetic
guuuauuucucuacuugaauucauacugac

SEQ ID NO: 620 - RNA, 30 nt, Artificial Sequence
  Synthetic
auuucucuacuugaauucauacugacuuug

SEQ ID NO: 621 - RNA, 30 nt, Artificial Sequence
  Synthetic
uuucucuacuugaauucauacugacuuugu

SEQ ID NO: 622 - RNA, 30 nt, Artificial Sequence
  Synthetic
cucuacuugaauucauacugacuuugugau

SEQ ID NO: 623 - RNA, 30 nt, Artificial Sequence
  Synthetic
cugcauauguggcuaaagcaauagacccau

SEQ ID NO: 624 - RNA, 30 nt, Artificial Sequence
  Synthetic
cuuauuuuaggaugaaaaauuuugaaacca

SEQ ID NO: 625 - RNA, 30 nt, Artificial Sequence
  Synthetic
gagacgacaguggacuugcugcauaugugg

SEQ ID NO: 626 - RNA, 30 nt, Artificial Sequence
  Synthetic
uggagacgacaguggacuugcugcauaugu

SEQ ID NO: 627 - RNA, 30 nt, Artificial Sequence
  Synthetic
uccugaggaaaauuuuggagacgacagugg

SEQ ID NO: 628 - RNA, 30 nt, Artificial Sequence
  Synthetic
cuccugaggaaaauuuuggagacgacagug

SEQ ID NO: 629 - RNA, 30 nt, Artificial Sequence
  Synthetic
ucuccugaggaaaauuuuggagacgacagu

SEQ ID NO: 630 - RNA, 30 nt, Artificial Sequence
  Synthetic
ucauuuucuccugaggaaaauuuuggagac

SEQ ID NO: 631 - RNA, 30 nt, Artificial Sequence
  Synthetic
aucauuuucuccugaggaaaauuuuggaga

SEQ ID NO: 632 - RNA, 30 nt, Artificial Sequence
  Synthetic
aaaccaguacuuuaucauuuucuccugagg

SEQ ID NO: 633 - RNA, 30 nt, Artificial Sequence
  Synthetic
gaaaccaguacuuuaucauuuucuccugag

SEQ ID NO: 634 - RNA, 30 nt, Artificial Sequence
  Synthetic
ugaaaccaguacuuuaucauuuucuccuga

SEQ ID NO: 635 - RNA, 30 nt, Artificial Sequence
  Synthetic
aggaugaaaaauuuugaaaccaguacuuua

SEQ ID NO: 636 - RNA, 30 nt, Artificial Sequence
  Synthetic
uaggaugaaaaauuuugaaaccaguacuuu

SEQ ID NO: 637 - RNA, 30 nt, Artificial Sequence
  Synthetic
uuuuaggaugaaaaauuuugaaaccaguac

SEQ ID NO: 638 - RNA, 30 nt, Artificial Sequence
  Synthetic
ggagacgacaguggacuugcugcauaugug

SEQ ID NO: 639 - RNA, 30 nt, Artificial Sequence
  Synthetic
caacaauuggcaaugaugucagucuucuca

SEQ ID NO: 640 - RNA, 29 nt, Artificial Sequence
  Synthetic
aauucauacugacuuugugauccuuugug

SEQ ID NO: 641 - RNA, 24 nt, Artificial Sequence
  Synthetic
auacugacuuugugauccuuugug

SEQ ID NO: 642 - RNA, 30 nt, Artificial Sequence
  Synthetic
aguuacaguuucccuaaggugcuuguuuua

SEQ ID NO: 643 - RNA, 30 nt, Artificial Sequence
  Synthetic
aagccauguuuaacagccucccuggcauca

SEQ ID NO: 644 - RNA, 30 nt, Artificial Sequence
  Synthetic
acagccucccuggcaucaucaccuggagag

SEQ ID NO: 645 - RNA, 30 nt, Artificial Sequence
  Synthetic
aacagccucccuggcaucaucaccuggaga

SEQ ID NO: 646 - RNA, 30 nt, Artificial Sequence
  Synthetic
gacaccaagaucccauucaagccauguuua

SEQ ID NO: 647 - RNA, 30 nt, Artificial Sequence
  Synthetic
ucgagccccaugauucgacaccaagauccc

SEQ ID NO: 648 - RNA, 30 nt, Artificial Sequence
  Synthetic
gcgucugccaaaacucacaguggcuggcac

SEQ ID NO: 649 - RNA, 30 nt, Artificial Sequence
  Synthetic
gcagacgcuaagauuuccuuuuggaguucc

SEQ ID NO: 650 - RNA, 30 nt, Artificial Sequence
  Synthetic
uggcagacgcuaagauuuccuuuuggaguu

SEQ ID NO: 651 - RNA, 30 nt, Artificial Sequence
  Synthetic
gugucgaaucauggggcucgacaacucgau

SEQ ID NO: 652 - RNA, 30 nt, Artificial Sequence
  Synthetic
ggcagacgcuaagauuuccuuuuggaguuc

SEQ ID NO: 653 - RNA, 30 nt, Artificial Sequence
  Synthetic
aacauggcuugaaugggaucuuggugucga

SEQ ID NO: 654 - RNA, 30 nt, Artificial Sequence
  Synthetic
cucucuccaggugaugaugccagggaggcu

SEQ ID NO: 655 - RNA, 30 nt, Artificial Sequence
  Synthetic
acucucuccaggugaugaugccagggaggc

SEQ ID NO: 656 - RNA, 30 nt, Artificial Sequence
  Synthetic
uacucucuccaggugaugaugccagggagg

SEQ ID NO: 657 - RNA, 30 nt, Artificial Sequence
  Synthetic
uuuuacucucuccaggugaugaugccaggg

SEQ ID NO: 658 - RNA, 30 nt, Artificial Sequence
  Synthetic
ccuaaggugcuuguuuuacucucuccaggu

SEQ ID NO: 659 - RNA, 30 nt, Artificial Sequence
  Synthetic
cccuaaggugcuuguuuuacucucuccagg

SEQ ID NO: 660 - RNA, 30 nt, Artificial Sequence
  Synthetic
caguuucccuaaggugcuuguuuuacucuc

SEQ ID NO: 661 - RNA, 30 nt, Artificial Sequence
  Synthetic
aaugggaucuuggugucgaaucauggggcu

SEQ ID NO: 662 - RNA, 24 nt, Artificial Sequence
  Synthetic
ccuuuuggaguucccauuuccauc

SEQ ID NO: 663 - RNA, 23 nt, Artificial Sequence
  Synthetic
cuuuuggaguucccauuuccauc

SEQ ID NO: 664 - RNA, 24 nt, Artificial Sequence
  Synthetic
gggaaacuguaacuuaacaggcag

SEQ ID NO: 665 - RNA, 30 nt, Artificial Sequence
  Synthetic
caacuuucuuuucuuuuaugaucuuuaagu

SEQ ID NO: 666 - RNA, 30 nt, Artificial Sequence
  Synthetic
cgguuggccauggcucugagugguaagacu

SEQ ID NO: 667 - RNA, 30 nt, Artificial Sequence
  Synthetic
gccauggcucugagugguaagacucauucu

SEQ ID NO: 668 - RNA, 30 nt, Artificial Sequence
  Synthetic
uuguuuacaacuuucuuuucuuuuaugauc

SEQ ID NO: 669 - RNA, 30 nt, Artificial Sequence
  Synthetic
uuuacaacuuucuuuucuuuuaugaucuuu

SEQ ID NO: 670 - RNA, 30 nt, Artificial Sequence
  Synthetic
acaacuuucuuuucuuuuaugaucuuuaag

SEQ ID NO: 671 - RNA, 30 nt, Artificial Sequence
  Synthetic
aagaucauaaaagaaaagaaaguuguaaac

SEQ ID NO: 672 - RNA, 30 nt, Artificial Sequence
  Synthetic
ucuauuuuauauauucauuucuuuguccag

SEQ ID NO: 673 - RNA, 30 nt, Artificial Sequence
  Synthetic
ccacucagagccauggccaaccggaauucu

SEQ ID NO: 674 - RNA, 30 nt, Artificial Sequence
  Synthetic
uuccuuuaguaucucgaggacaucuugaac

SEQ ID NO: 675 - RNA, 30 nt, Artificial Sequence
  Synthetic
cuuuaguaucucgaggacaucuugaacacc

SEQ ID NO: 676 - RNA, 30 nt, Artificial Sequence
  Synthetic
aguaucucgaggacaucuugaacaccuuuc

SEQ ID NO: 677 - RNA, 30 nt, Artificial Sequence
  Synthetic
guaucucgaggacaucuugaacaccuuucu

SEQ ID NO: 678 - RNA, 30 nt, Artificial Sequence
  Synthetic
aagauguccucgagauacuaaaggaagaau

SEQ ID NO: 679 - RNA, 30 nt, Artificial Sequence
  Synthetic
uaaacaagaaugagucuuaccacucagagc

SEQ ID NO: 680 - RNA, 30 nt, Artificial Sequence
  Synthetic
gggggagaaagguguucaagauguccucga

SEQ ID NO: 681 - RNA, 30 nt, Artificial Sequence
  Synthetic
ccagguaacuggacaaagaaaugaauauau

SEQ ID NO: 682 - RNA, 30 nt, Artificial Sequence
  Synthetic
acuugguuagggggagaaagguguucaaga

SEQ ID NO: 683 - RNA, 30 nt, Artificial Sequence
  Synthetic
cacuugguuagggggagaaagguguucaag

SEQ ID NO: 684 - RNA, 30 nt, Artificial Sequence
  Synthetic
ucacuugguuagggggagaaagguguucaa

SEQ ID NO: 685 - RNA, 30 nt, Artificial Sequence
  Synthetic
ugaaucacucuguaucuuuucacuugguua

SEQ ID NO: 686 - RNA, 30 nt, Artificial Sequence
  Synthetic
guucugaaucacucuguaucuuuucacuug

SEQ ID NO: 687 - RNA, 30 nt, Artificial Sequence
  Synthetic
aguucugaaucacucuguaucuuuucacuu

SEQ ID NO: 688 - RNA, 30 nt, Artificial Sequence
  Synthetic
acaguaaaacaaaugaauaaaacaagucag

SEQ ID NO: 689 - RNA, 30 nt, Artificial Sequence
  Synthetic
cagguaacuggacaaagaaaugaauauaua

SEQ ID NO: 690 - RNA, 30 nt, Artificial Sequence
  Synthetic
aacaccuuucucccccuaaccaagugaaaa

SEQ ID NO: 691 - RNA, 30 nt, Artificial Sequence
  Synthetic
gcuuuccagguaacuggacaaagaaaugaa

SEQ ID NO: 692 - RNA, 30 nt, Artificial Sequence
  Synthetic
gggcuuagcuuuccagguaacuggacaaag

SEQ ID NO: 693 - RNA, 30 nt, Artificial Sequence
  Synthetic
ggggcuuagcuuuccagguaacuggacaaa

SEQ ID NO: 694 - RNA, 30 nt, Artificial Sequence
  Synthetic
uggggagaccaaucguuuggggcuuagcuu

SEQ ID NO: 695 - RNA, 30 nt, Artificial Sequence
  Synthetic
guggggagaccaaucguuuggggcuuagcu

SEQ ID NO: 696 - RNA, 30 nt, Artificial Sequence
  Synthetic
guuagggggagaaagguguucaagaugucc

SEQ ID NO: 697 - RNA, 30 nt, Artificial Sequence
  Synthetic
cucccccuaaccaagugaaaagauacagag

SEQ ID NO: 698 - RNA, 30 nt, Artificial Sequence
  Synthetic
uacugucaaguugucuauuuuauauauuca

SEQ ID NO: 699 - RNA, 30 nt, Artificial Sequence
  Synthetic
agaacuaaaucagucugacuuguuuuauuc

SEQ ID NO: 700 - RNA, 30 nt, Artificial Sequence
  Synthetic
aauaaugugacucuauuaacacugaauugu

SEQ ID NO: 701 - RNA, 30 nt, Artificial Sequence
  Synthetic
uggcagaacaucaaucuggggaaagaaaag

SEQ ID NO: 702 - RNA, 30 nt, Artificial Sequence
  Synthetic
cuggcagaacaucaaucuggggaaagaaaa

SEQ ID NO: 703 - RNA, 30 nt, Artificial Sequence
  Synthetic
cacagccuccacaauuucuggcagaacauc

SEQ ID NO: 704 - RNA, 30 nt, Artificial Sequence
  Synthetic
ccuuccacagccuccacaauuucuggcaga

SEQ ID NO: 705 - RNA, 30 nt, Artificial Sequence
  Synthetic
caccuucccuuccacagccuccacaauuuc

SEQ ID NO: 706 - RNA, 30 nt, Artificial Sequence
  Synthetic
cgcacacccccguccaggaagacuuccacc

SEQ ID NO: 707 - RNA, 30 nt, Artificial Sequence
  Synthetic
ccgcacacccccguccaggaagacuuccac

SEQ ID NO: 708 - RNA, 30 nt, Artificial Sequence
  Synthetic
agaacaucagugccuuuccgcacacccccg

SEQ ID NO: 709 - RNA, 30 nt, Artificial Sequence
  Synthetic
cagaacaucagugccuuuccgcacaccccc

SEQ ID NO: 710 - RNA, 30 nt, Artificial Sequence
  Synthetic
gcgccaagagccagagcuuucagaacauca

SEQ ID NO: 711 - RNA, 30 nt, Artificial Sequence
  Synthetic
gucuccccacaaacacagccuuggcgccaa

SEQ ID NO: 712 - RNA, 30 nt, Artificial Sequence
  Synthetic
ccuggaaagcuaagccccaaacgauugguc

SEQ ID NO: 713 - RNA, 30 nt, Artificial Sequence
  Synthetic
uccaguuaccuggaaagcuaagccccaaac

SEQ ID NO: 714 - RNA, 30 nt, Artificial Sequence
  Synthetic
guccaguuaccuggaaagcuaagccccaaa

SEQ ID NO: 715 - RNA, 30 nt, Artificial Sequence
  Synthetic
uuuguccaguuaccuggaaagcuaagcccc

SEQ ID NO: 716 - RNA, 30 nt, Artificial Sequence
  Synthetic
cuuuguccaguuaccuggaaagcuaagccc

SEQ ID NO: 717 - RNA, 30 nt, Artificial Sequence
  Synthetic
uuuuauucauuuguuuuacugucaaguugu

SEQ ID NO: 718 - RNA, 30 nt, Artificial Sequence
  Synthetic
uauucauuuguuuuacugucaaguugucua

SEQ ID NO: 719 - RNA, 30 nt, Artificial Sequence
  Synthetic
auucauuuguuuuacugucaaguugucuau

SEQ ID NO: 720 - RNA, 30 nt, Artificial Sequence
  Synthetic
uucauuuguuuuacugucaaguugucuauu

SEQ ID NO: 721 - RNA, 30 nt, Artificial Sequence
  Synthetic
auuuguuuuacugucaaguugucuauuuua

SEQ ID NO: 722 - RNA, 30 nt, Artificial Sequence
  Synthetic
guuuuacugucaaguugucuauuuuauaua

SEQ ID NO: 723 - RNA, 30 nt, Artificial Sequence
  Synthetic
ucccccuaaccaagugaaaagauacagagu

SEQ ID NO: 724 - RNA, 30 nt, Artificial Sequence
  Synthetic
uuuuacugucaaguugucuauuuuauauau

SEQ ID NO: 725 - RNA, 30 nt, Artificial Sequence
  Synthetic
acugucaaguugucuauuuuauauauucau

SEQ ID NO: 726 - RNA, 30 nt, Artificial Sequence
  Synthetic
cugucaaguugucuauuuuauauauucauu

SEQ ID NO: 727 - RNA, 30 nt, Artificial Sequence
  Synthetic
uauauauucauuucuuuguccaguuaccug

SEQ ID NO: 728 - RNA, 30 nt, Artificial Sequence
  Synthetic
auauauucauuucuuuguccaguuaccugg

SEQ ID NO: 729 - RNA, 30 nt, Artificial Sequence
  Synthetic
uauauucauuucuuuguccaguuaccugga

SEQ ID NO: 730 - RNA, 30 nt, Artificial Sequence
  Synthetic
auuucuuuguccaguuaccuggaaagcuaa

SEQ ID NO: 731 - RNA, 30 nt, Artificial Sequence
  Synthetic
gcgccaaggcuguguuuguggggagaccaa

SEQ ID NO: 732 - RNA, 30 nt, Artificial Sequence
  Synthetic
ugaaagcucuggcucuuggcgccaaggcug

SEQ ID NO: 733 - RNA, 30 nt, Artificial Sequence
  Synthetic
uggaggcuguggaagggaagguggaagucu

SEQ ID NO: 734 - RNA, 30 nt, Artificial Sequence
  Synthetic
uugaacuuuucuuuccccagauugauguuc

SEQ ID NO: 735 - RNA, 30 nt, Artificial Sequence
  Synthetic
ugccagaaauuguggaggcuguggaaggga

SEQ ID NO: 736 - RNA, 30 nt, Artificial Sequence
  Synthetic
auguucugccagaaauuguggaggcugugg

SEQ ID NO: 737 - RNA, 30 nt, Artificial Sequence
  Synthetic
cccagauugauguucugccagaaauugugg

SEQ ID NO: 738 - RNA, 30 nt, Artificial Sequence
  Synthetic
ccccagauugauguucugccagaaauugug

SEQ ID NO: 739 - RNA, 30 nt, Artificial Sequence
  Synthetic
uuuccccagauugauguucugccagaaauu

SEQ ID NO: 740 - RNA, 30 nt, Artificial Sequence
  Synthetic
cuuuccccagauugauguucugccagaaau

SEQ ID NO: 741 - RNA, 30 nt, Artificial Sequence
  Synthetic
ucuuuccccagauugauguucugccagaaa

SEQ ID NO: 742 - RNA, 30 nt, Artificial Sequence
  Synthetic
aacuuuucuuuccccagauugauguucugc

SEQ ID NO: 743 - RNA, 30 nt, Artificial Sequence
  Synthetic
auagagucacauuauugaacuuuucuuucc

SEQ ID NO: 744 - RNA, 30 nt, Artificial Sequence
  Synthetic
aguguuaauagagucacauuauugaacuuu

SEQ ID NO: 745 - RNA, 30 nt, Artificial Sequence
  Synthetic
cuggacgggggugugcggaaaggcacugau

SEQ ID NO: 746 - RNA, 23 nt, Artificial Sequence
  Synthetic
cuuuucuuuuaugaucuuuaagu

SEQ ID NO: 747 - RNA, 22 nt, Artificial Sequence
  Synthetic
uuuucuuuuaugaucuuuaagu

SEQ ID NO: 748 - RNA, 30 nt, Artificial Sequence
  Synthetic
uuucagggugccagaaugugaaagucaucg

SEQ ID NO: 749 - RNA, 30 nt, Artificial Sequence
  Synthetic
uuuuuucagggugccagaaugugaaaguca

SEQ ID NO: 750 - RNA, 30 nt, Artificial Sequence
  Synthetic
uaagcucagguucaaaguguugguaaugcc

SEQ ID NO: 751 - RNA, 30 nt, Artificial Sequence
  Synthetic
auauuaaauguaugcauuauuuuuucaggg

SEQ ID NO: 752 - RNA, 30 nt, Artificial Sequence
  Synthetic
aguucauauuaaauguaugcauuauuuuuu

SEQ ID NO: 753 - RNA, 30 nt, Artificial Sequence
  Synthetic
uucagggugccagaaugugaaagucaucga

SEQ ID NO: 754 - RNA, 30 nt, Artificial Sequence
  Synthetic
aauguaugcauuauuuuuucagggugccag

SEQ ID NO: 755 - RNA, 30 nt, Artificial Sequence
  Synthetic
ucagggugccagaaugugaaagucaucgac

SEQ ID NO: 756 - RNA, 30 nt, Artificial Sequence
  Synthetic
gccguuuccaagaucugacagugcacaaua

SEQ ID NO: 757 - RNA, 30 nt, Artificial Sequence
  Synthetic
agggugccagaaugugaaagucaucgacaa

SEQ ID NO: 758 - RNA, 30 nt, Artificial Sequence
  Synthetic
gugaggaaaaauccuuuggccguuuccaag

SEQ ID NO: 759 - RNA, 30 nt, Artificial Sequence
  Synthetic
ggccguuuccaagaucugacagugcacaau

SEQ ID NO: 760 - RNA, 30 nt, Artificial Sequence
  Synthetic
cauucaguucauauuaaauguaugcauuau

SEQ ID NO: 761 - RNA, 30 nt, Artificial Sequence
  Synthetic
ccaagaucugacagugcacaauauuuuccc

SEQ ID NO: 762 - RNA, 30 nt, Artificial Sequence
  Synthetic
caagaucugacagugcacaauauuuuccca

SEQ ID NO: 763 - RNA, 30 nt, Artificial Sequence
  Synthetic
cagggugccagaaugugaaagucaucgaca

SEQ ID NO: 764 - RNA, 30 nt, Artificial Sequence
  Synthetic
uugcauucaguucauauuaaauguaugcau

SEQ ID NO: 765 - RNA, 30 nt, Artificial Sequence
  Synthetic
gagguagcaaacacuaaggugaaaagauaa

SEQ ID NO: 766 - RNA, 30 nt, Artificial Sequence
  Synthetic
agacaacgucauccccuggcaggcuaaagu

SEQ ID NO: 767 - RNA, 30 nt, Artificial Sequence
  Synthetic
aauuguaagcucagguucaaaguguuggua

SEQ ID NO: 768 - RNA, 30 nt, Artificial Sequence
  Synthetic
uuaaauuguaagcucagguucaaaguguug

SEQ ID NO: 769 - RNA, 30 nt, Artificial Sequence
  Synthetic
aaacagugguucuuaaauuguaagcucagg

SEQ ID NO: 770 - RNA, 30 nt, Artificial Sequence
  Synthetic
aaaacagugguucuuaaauuguaagcucag

SEQ ID NO: 771 - RNA, 30 nt, Artificial Sequence
  Synthetic
caugucuuuaaaacagugguucuuaaauug

SEQ ID NO: 772 - RNA, 30 nt, Artificial Sequence
  Synthetic
acaugucuuuaaaacagugguucuuaaauu

SEQ ID NO: 773 - RNA, 30 nt, Artificial Sequence
  Synthetic
uguuuacaugucuuuaaaacagugguucuu

SEQ ID NO: 774 - RNA, 30 nt, Artificial Sequence
  Synthetic
accuguauucuguuuacaugucuuuaaaac

SEQ ID NO: 775 - RNA, 30 nt, Artificial Sequence
  Synthetic
uuaaccuguauucuguuuacaugucuuuaa

SEQ ID NO: 776 - RNA, 30 nt, Artificial Sequence
  Synthetic
auuaaccuguauucuguuuacaugucuuua

SEQ ID NO: 777 - RNA, 30 nt, Artificial Sequence
  Synthetic
uuuauuaaccuguauucuguuuacaugucu

SEQ ID NO: 778 - RNA, 30 nt, Artificial Sequence
  Synthetic
ucccaucuguauuauuuuuuuucagcaugu

SEQ ID NO: 779 - RNA, 30 nt, Artificial Sequence
  Synthetic
guaaaauuggagguagcaaacacuaaggug

SEQ ID NO: 780 - RNA, 30 nt, Artificial Sequence
  Synthetic
aguaaaauuggagguagcaaacacuaaggu

SEQ ID NO: 781 - RNA, 30 nt, Artificial Sequence
  Synthetic
gacaacgucauccccuggcaggcuaaagug

SEQ ID NO: 782 - RNA, 30 nt, Artificial Sequence
  Synthetic
uuauugcauucaguucauauuaaauguaug

SEQ ID NO: 783 - RNA, 30 nt, Artificial Sequence
  Synthetic
cccaucuguauuauuuuuuuucagcaugua

SEQ ID NO: 784 - RNA, 30 nt, Artificial Sequence
  Synthetic
uucagcauguauuacuugacaaagagacac

SEQ ID NO: 785 - RNA, 30 nt, Artificial Sequence
  Synthetic
uuuuuuuucagcauguauuacuugacaaag

SEQ ID NO: 786 - RNA, 30 nt, Artificial Sequence
  Synthetic
auugcuuuugacuuuucaauggguguccua

SEQ ID NO: 787 - RNA, 30 nt, Artificial Sequence
  Synthetic
cuuuugacuuuucaauggguguccuaggaa

SEQ ID NO: 788 - RNA, 30 nt, Artificial Sequence
  Synthetic
ugacuuuucaauggguguccuaggaaccuu

SEQ ID NO: 789 - RNA, 30 nt, Artificial Sequence
  Synthetic
gacuuuucaauggguguccuaggaaccuuu

SEQ ID NO: 790 - RNA, 30 nt, Artificial Sequence
  Synthetic
acuuuucaauggguguccuaggaaccuuuu

SEQ ID NO: 791 - RNA, 30 nt, Artificial Sequence
  Synthetic
ucaauggguguccuaggaaccuuuuagaaa

SEQ ID NO: 792 - RNA, 30 nt, Artificial Sequence
  Synthetic
caauggguguccuaggaaccuuuuagaaag

SEQ ID NO: 793 - RNA, 30 nt, Artificial Sequence
  Synthetic
aauggguguccuaggaaccuuuuagaaaga

SEQ ID NO: 794 - RNA, 30 nt, Artificial Sequence
  Synthetic
uagaaagaaauggacuuucauccuggaaau

SEQ ID NO: 795 - RNA, 30 nt, Artificial Sequence
  Synthetic
agaaagaaauggacuuucauccuggaaaua

SEQ ID NO: 796 - RNA, 30 nt, Artificial Sequence
  Synthetic
gaaagaaauggacuuucauccuggaaauau

SEQ ID NO: 797 - RNA, 30 nt, Artificial Sequence
  Synthetic
cauccuggaaauauauuaacuguuaaaaag

SEQ ID NO: 798 - RNA, 30 nt, Artificial Sequence
  Synthetic
auccuggaaauauauuaacuguuaaaaaga

SEQ ID NO: 799 - RNA, 30 nt, Artificial Sequence
  Synthetic
acuguuaaaaagaaaacauugaaaaugugu

SEQ ID NO: 800 - RNA, 30 nt, Artificial Sequence
  Synthetic
aaaagaaaacauugaaaauguguuuagaca

SEQ ID NO: 801 - RNA, 30 nt, Artificial Sequence
  Synthetic
cauugcuuuugacuuuucaauggguguccu

SEQ ID NO: 802 - RNA, 30 nt, Artificial Sequence
  Synthetic
ccaucuguauuauuuuuuuucagcauguau

SEQ ID NO: 803 - RNA, 30 nt, Artificial Sequence
  Synthetic
uuucauugcuuuugacuuuucaaugggugu

SEQ ID NO: 804 - RNA, 30 nt, Artificial Sequence
  Synthetic
uauuucauugcuuuugacuuuucaaugggu

SEQ ID NO: 805 - RNA, 30 nt, Artificial Sequence
  Synthetic
aaaauguguuuagacaacgucauccccugg

SEQ ID NO: 806 - RNA, 30 nt, Artificial Sequence
  Synthetic
uuuuucagcauguauuacuugacaaagaga

SEQ ID NO: 807 - RNA, 30 nt, Artificial Sequence
  Synthetic
uuuucagcauguauuacuugacaaagagac

SEQ ID NO: 808 - RNA, 30 nt, Artificial Sequence
  Synthetic
uuucagcauguauuacuugacaaagagaca

SEQ ID NO: 809 - RNA, 30 nt, Artificial Sequence
  Synthetic
ucagcauguauuacuugacaaagagacacu

SEQ ID NO: 810 - RNA, 30 nt, Artificial Sequence
  Synthetic
cagcauguauuacuugacaaagagacacug

SEQ ID NO: 811 - RNA, 30 nt, Artificial Sequence
  Synthetic
agcauguauuacuugacaaagagacacugu

SEQ ID NO: 812 - RNA, 30 nt, Artificial Sequence
  Synthetic
cuugacaaagagacacugugcagaggguga

SEQ ID NO: 813 - RNA, 30 nt, Artificial Sequence
  Synthetic
acaaagagacacugugcagagggugaccac

SEQ ID NO: 814 - RNA, 30 nt, Artificial Sequence
  Synthetic
cccacuucaauacaaagggugucguucuuu

SEQ ID NO: 815 - RNA, 30 nt, Artificial Sequence
  Synthetic
aauacaaagggugucguucuuuuccaacaa

SEQ ID NO: 816 - RNA, 30 nt, Artificial Sequence
  Synthetic
uuuuccaacaaaauagcaaucccuuuuauu

SEQ ID NO: 817 - RNA, 30 nt, Artificial Sequence
  Synthetic
uccaacaaaauagcaaucccuuuuauuuca

SEQ ID NO: 818 - RNA, 30 nt, Artificial Sequence
  Synthetic
ccaacaaaauagcaaucccuuuuauuucau

SEQ ID NO: 819 - RNA, 30 nt, Artificial Sequence
  Synthetic
caacaaaauagcaaucccuuuuauuucauu

SEQ ID NO: 820 - RNA, 30 nt, Artificial Sequence
  Synthetic
auuucauugcuuuugacuuuucaaugggug

SEQ ID NO: 821 - RNA, 30 nt, Artificial Sequence
  Synthetic
aaaguguugguaaugccugauucacaacuu

SEQ ID NO: 822 - RNA, 30 nt, Artificial Sequence
  Synthetic
cucucuaagaaguaacauacauccuaaaac

SEQ ID NO: 823 - RNA, 30 nt, Artificial Sequence
  Synthetic
acaacuuugagaagguagcacuggagagaa

SEQ ID NO: 824 - RNA, 30 nt, Artificial Sequence
  Synthetic
accuuaguguuugcuaccuccaauuuuacu

SEQ ID NO: 825 - RNA, 30 nt, Artificial Sequence
  Synthetic
guguuugcuaccuccaauuuuacuaaagga

SEQ ID NO: 826 - RNA, 30 nt, Artificial Sequence
  Synthetic
gcuaccuccaauuuuacuaaaggauacagc

SEQ ID NO: 827 - RNA, 30 nt, Artificial Sequence
  Synthetic
cuaccuccaauuuuacuaaaggauacagca

SEQ ID NO: 828 - RNA, 30 nt, Artificial Sequence
  Synthetic
uacuaaaggauacagcacuuuagccugcca

SEQ ID NO: 829 - RNA, 30 nt, Artificial Sequence
  Synthetic
acuaaaggauacagcacuuuagccugccag

SEQ ID NO: 830 - RNA, 30 nt, Artificial Sequence
  Synthetic
cuaaaggauacagcacuuuagccugccagg

SEQ ID NO: 831 - RNA, 30 nt, Artificial Sequence
  Synthetic
agccugccaggggaugacguugucuaaaca

SEQ ID NO: 832 - RNA, 30 nt, Artificial Sequence
  Synthetic
gccugccaggggaugacguugucuaaacac

SEQ ID NO: 833 - RNA, 30 nt, Artificial Sequence
  Synthetic
caccuuaguguuugcuaccuccaauuuuac

SEQ ID NO: 834 - RNA, 30 nt, Artificial Sequence
  Synthetic
ucuaaacacauuuucaauguuuucuuuuua

SEQ ID NO: 835 - RNA, 30 nt, Artificial Sequence
  Synthetic
caauguuuucuuuuuaacaguuaauauauu

SEQ ID NO: 836 - RNA, 30 nt, Artificial Sequence
  Synthetic
aauguuuucuuuuuaacaguuaauauauuu

SEQ ID NO: 837 - RNA, 30 nt, Artificial Sequence
  Synthetic
ucuuuuuaacaguuaauauauuuccaggau

SEQ ID NO: 838 - RNA, 30 nt, Artificial Sequence
  Synthetic
cuuuuuaacaguuaauauauuuccaggaug

SEQ ID NO: 839 - RNA, 30 nt, Artificial Sequence
  Synthetic
uuuuuaacaguuaauauauuuccaggauga

SEQ ID NO: 840 - RNA, 30 nt, Artificial Sequence
  Synthetic
uuaacaguuaauauauuuccaggaugaaag

SEQ ID NO: 841 - RNA, 30 nt, Artificial Sequence
  Synthetic
uaacaguuaauauauuuccaggaugaaagu

SEQ ID NO: 842 - RNA, 30 nt, Artificial Sequence
  Synthetic
aacaguuaauauauuuccaggaugaaaguc

SEQ ID NO: 843 - RNA, 30 nt, Artificial Sequence
  Synthetic
acaguuaauauauuuccaggaugaaagucc

SEQ ID NO: 844 - RNA, 30 nt, Artificial Sequence
  Synthetic
ucaauguuuucuuuuuaacaguuaauauau

SEQ ID NO: 845 - RNA, 30 nt, Artificial Sequence
  Synthetic
auauauuuccaggaugaaaguccauuucuu

SEQ ID NO: 846 - RNA, 30 nt, Artificial Sequence
  Synthetic
ucaccuuaguguuugcuaccuccaauuuua

SEQ ID NO: 847 - RNA, 30 nt, Artificial Sequence
  Synthetic
auaaacaaugagaucauuaucuuuucaccu

SEQ ID NO: 848 - RNA, 30 nt, Artificial Sequence
  Synthetic
ucucuaagaaguaacauacauccuaaaaca

SEQ ID NO: 849 - RNA, 30 nt, Artificial Sequence
  Synthetic
ggauauauucagacacuaaagaugugauug

SEQ ID NO: 850 - RNA, 30 nt, Artificial Sequence
  Synthetic
gauauauucagacacuaaagaugugauugg

SEQ ID NO: 851 - RNA, 30 nt, Artificial Sequence
  Synthetic
agacacuaaagaugugauuggaaaucuaca

SEQ ID NO: 852 - RNA, 30 nt, Artificial Sequence
  Synthetic
gaaaucuacauucaaagaaguaucaccaau

SEQ ID NO: 853 - RNA, 30 nt, Artificial Sequence
  Synthetic
aaagaaguaucaccaauuaccgccacccau

SEQ ID NO: 854 - RNA, 30 nt, Artificial Sequence
  Synthetic
ccgccacccauuccaauucucuccagugcu

SEQ ID NO: 855 - RNA, 30 nt, Artificial Sequence
  Synthetic
caauucucuccagugcuaccuucucaaagu

SEQ ID NO: 856 - RNA, 30 nt, Artificial Sequence
  Synthetic
ucuccagugcuaccuucucaaaguugugaa

SEQ ID NO: 857 - RNA, 30 nt, Artificial Sequence
  Synthetic
ucuuuucaccuuaguguuugcuaccuccaa

SEQ ID NO: 858 - RNA, 30 nt, Artificial Sequence
  Synthetic
ucaaaguugugaaucaggcauuaccaacac

SEQ ID NO: 859 - RNA, 30 nt, Artificial Sequence
  Synthetic
ccaacacuuugaaccugagcuuacaauuua

SEQ ID NO: 860 - RNA, 30 nt, Artificial Sequence
  Synthetic
gaaccugagcuuacaauuuaagaaccacug

SEQ ID NO: 861 - RNA, 30 nt, Artificial Sequence
  Synthetic
aaccugagcuuacaauuuaagaaccacugu

SEQ ID NO: 862 - RNA, 30 nt, Artificial Sequence
  Synthetic
caauuuaagaaccacuguuuuaaagacaug

SEQ ID NO: 863 - RNA, 30 nt, Artificial Sequence
  Synthetic
aagaaccacuguuuuaaagacauguaaaca

SEQ ID NO: 864 - RNA, 30 nt, Artificial Sequence
  Synthetic
agaaccacuguuuuaaagacauguaaacag

SEQ ID NO: 865 - RNA, 30 nt, Artificial Sequence
  Synthetic
uaaagacauguaaacagaauacagguuaau

SEQ ID NO: 866 - RNA, 30 nt, Artificial Sequence
  Synthetic
aaagacauguaaacagaauacagguuaaua

SEQ ID NO: 867 - RNA, 30 nt, Artificial Sequence
  Synthetic
aagacauguaaacagaauacagguuaauaa

SEQ ID NO: 868 - RNA, 30 nt, Artificial Sequence
  Synthetic
ugaaucaggcauuaccaacacuuugaaccu

SEQ ID NO: 869 - RNA, 30 nt, Artificial Sequence
  Synthetic
guaaugccugauucacaacuuugagaaggu

SEQ ID NO: 870 - RNA, 30 nt, Artificial Sequence
  Synthetic
ccaggaugaaaguccauuucuuucuaaaag

SEQ ID NO: 871 - RNA, 30 nt, Artificial Sequence
  Synthetic
auuucucucuaagaaguaacauacauccua

SEQ ID NO: 872 - RNA, 30 nt, Artificial Sequence
  Synthetic
acauucuggcacccugaaaaaauaaugcau

SEQ ID NO: 873 - RNA, 30 nt, Artificial Sequence
  Synthetic
uggcacccugaaaaaauaaugcauacauuu

SEQ ID NO: 874 - RNA, 30 nt, Artificial Sequence
  Synthetic
uuucucucuaagaaguaacauacauccuaa

SEQ ID NO: 875 - RNA, 30 nt, Artificial Sequence
  Synthetic
ccaaaaaugcuuuauuucucucuaagaagu

SEQ ID NO: 876 - RNA, 30 nt, Artificial Sequence
  Synthetic
uuagagagaaauaaagcauuuuugggaaga

SEQ ID NO: 877 - RNA, 30 nt, Artificial Sequence
  Synthetic
cuucuuagagagaaauaaagcauuuuuggg

SEQ ID NO: 878 - RNA, 30 nt, Artificial Sequence
  Synthetic
ggauguauguuacuucuuagagagaaauaa

SEQ ID NO: 879 - RNA, 30 nt, Artificial Sequence
  Synthetic
aggauguauguuacuucuuagagagaaaua

SEQ ID NO: 880 - RNA, 30 nt, Artificial Sequence
  Synthetic
uaggauguauguuacuucuuagagagaaau

SEQ ID NO: 881 - RNA, 30 nt, Artificial Sequence
  Synthetic
cacauucuggcacccugaaaaaauaaugca

SEQ ID NO: 882 - RNA, 30 nt, Artificial Sequence
  Synthetic
gugucugaauauauccaaauguuuuaggau

SEQ ID NO: 883 - RNA, 30 nt, Artificial Sequence
  Synthetic
caaucacaucuuuagugucugaauauaucc

SEQ ID NO: 884 - RNA, 30 nt, Artificial Sequence
  Synthetic
ccaaucacaucuuuagugucugaauauauc

SEQ ID NO: 885 - RNA, 30 nt, Artificial Sequence
  Synthetic
aauguagauuuccaaucacaucuuuagugu

SEQ ID NO: 886 - RNA, 30 nt, Artificial Sequence
  Synthetic
gaauguagauuuccaaucacaucuuuagug

SEQ ID NO: 887 - RNA, 30 nt, Artificial Sequence
  Synthetic
uuugaauguagauuuccaaucacaucuuua

SEQ ID NO: 888 - RNA, 30 nt, Artificial Sequence
  Synthetic
gugauacuucuuugaauguagauuuccaau

SEQ ID NO: 889 - RNA, 30 nt, Artificial Sequence
  Synthetic
gaauggguggcgguaauuggugauacuucu

SEQ ID NO: 890 - RNA, 30 nt, Artificial Sequence
  Synthetic
agaagguagcacuggagagaauuggaaugg

SEQ ID NO: 891 - RNA, 30 nt, Artificial Sequence
  Synthetic
gagaagguagcacuggagagaauuggaaug

SEQ ID NO: 892 - RNA, 30 nt, Artificial Sequence
  Synthetic
agugucugaauauauccaaauguuuuagga

SEQ ID NO: 893 - RNA, 30 nt, Artificial Sequence
  Synthetic
caggaugaaaguccauuucuuucuaaaagg

SEQ ID NO: 894 - RNA, 30 nt, Artificial Sequence
  Synthetic
ucgaugacuuucacauucuggcacccugaa

SEQ ID NO: 895 - RNA, 30 nt, Artificial Sequence
  Synthetic
ccucaccaaugucuugucgaugacuuucac

SEQ ID NO: 896 - RNA, 30 nt, Artificial Sequence
  Synthetic
uuucuaaaagguuccuaggacacccauuga

SEQ ID NO: 897 - RNA, 30 nt, Artificial Sequence
  Synthetic
cuaaaagguuccuaggacacccauugaaaa

SEQ ID NO: 898 - RNA, 30 nt, Artificial Sequence
  Synthetic
uaaaagguuccuaggacacccauugaaaag

SEQ ID NO: 899 - RNA, 30 nt, Artificial Sequence
  Synthetic
cuaggacacccauugaaaagucaaaagcaa

SEQ ID NO: 900 - RNA, 30 nt, Artificial Sequence
  Synthetic
aaaagucaaaagcaaugaaauaaaagggau

SEQ ID NO: 901 - RNA, 30 nt, Artificial Sequence
  Synthetic
cuauuuuguuggaaaagaacgacacccuuu

SEQ ID NO: 902 - RNA, 30 nt, Artificial Sequence
  Synthetic
uguuggaaaagaacgacacccuuuguauug

SEQ ID NO: 903 - RNA, 30 nt, Artificial Sequence
  Synthetic
guuggaaaagaacgacacccuuuguauuga

SEQ ID NO: 904 - RNA, 30 nt, Artificial Sequence
  Synthetic
uuggaaaagaacgacacccuuuguauugaa

SEQ ID NO: 905 - RNA, 30 nt, Artificial Sequence
  Synthetic
cucaccaaugucuugucgaugacuuucaca

SEQ ID NO: 906 - RNA, 30 nt, Artificial Sequence
  Synthetic
gaaaagaacgacacccuuuguauugaagug

SEQ ID NO: 907 - RNA, 30 nt, Artificial Sequence
  Synthetic
uauugaaguggggaauuacagacugugguc

SEQ ID NO: 908 - RNA, 30 nt, Artificial Sequence
  Synthetic
aaguggggaauuacagacuguggucacccu

SEQ ID NO: 909 - RNA, 30 nt, Artificial Sequence
  Synthetic
cagacuguggucacccucugcacagugucu

SEQ ID NO: 910 - RNA, 30 nt, Artificial Sequence
  Synthetic
gucaaguaauacaugcugaaaaaaaauaau

SEQ ID NO: 911 - RNA, 30 nt, Artificial Sequence
  Synthetic
ucaaguaauacaugcugaaaaaaaauaaua

SEQ ID NO: 912 - RNA, 30 nt, Artificial Sequence
  Synthetic
ugcacugucagaucuuggaaacggccaaag

SEQ ID NO: 913 - RNA, 30 nt, Artificial Sequence
  Synthetic
gaaacggccaaaggauuuuuccucaccaau

SEQ ID NO: 914 - RNA, 30 nt, Artificial Sequence
  Synthetic
uuccucaccaaugucuugucgaugacuuuc

SEQ ID NO: 915 - RNA, 30 nt, Artificial Sequence
  Synthetic
uccucaccaaugucuugucgaugacuuuca

SEQ ID NO: 916 - RNA, 30 nt, Artificial Sequence
  Synthetic
guauugaaguggggaauuacagacuguggu

SEQ ID NO: 917 - RNA, 30 nt, Artificial Sequence
  Synthetic
cuuucuaaaagguuccuaggacacccauug

SEQ ID NO: 918 - RNA, 28 nt, Artificial Sequence
  Synthetic
gagagaaauaaagcauuuuugggaagaa

SEQ ID NO: 919 - RNA, 27 nt, Artificial Sequence
  Synthetic
aauaugaacugaaugcaauaauaauca

SEQ ID NO: 920 - RNA, 26 nt, Artificial Sequence
  Synthetic
auaugaacugaaugcaauaauaauca

SEQ ID NO: 921 - DNA, 3162 bp, Artificial Sequence
  Synthetic
atgagcagcgcgatcaaaagctacaagagcgttctgcgtccgaacgagcgtaagaaccaactgctgaaaagcaccattcagtgcctggaagacggtagcgcgttctttttcaagatgctgcaaggcctgtttggtggcatcaccccggagattgttcgtttcagcaccgaacaggagaaacagcaacaggatatcgcgctgtggtgcgcggttaactggttccgtccggtgagccaagacagcctgacccacaccattgcgagcgataacctggtggagaagtttgaggaatactatggtggcaccgcgagcgacgcgatcaaacagtacttcagcgcgagcattggcgaaagctactattggaacgactgccgtcaacagtactatgatctgtgccgtgagctgggtgttgaggtgagcgacctgacccatgatctggagatcctgtgccgtgaaaagtgcctggcggttgcgaccgagagcaaccagaacaacagcatcattagcgttctgtttggcaccggcgaaaaagaggaccgtagcgtgaaactgcgtatcaccaagaaaattctggaggcgatcagcaacctgaaagaaatcccgaagaacgttgcgccgattcaagagatcattctgaacgtggcgaaagcgaccaaggaaaccttccgtcaggtgtatgcgggtaacctgggtgcgccgagcaccctggagaaatttatcgcgaaggacggccaaaaagagttcgatctgaagaaactgcagaccgacctgaagaaagttattcgtggtaaaagcaaggagcgtgattggtgctgccaggaagagctgcgtagctacgtggagcaaaacaccatccagtatgacctgtgggcgtggggcgaaatgttcaacaaagcgcacaccgcgctgaaaatcaagagcacccgtaactacaactttgcgaagcaacgtctggaacagttcaaagagattcagagcctgaacaacctgctggttgtgaagaagctgaacgactttttcgatagcgaatttttcagcggcgaggaaacctacaccatctgcgttcaccatctgggtggcaaggacctgagcaaactgtataaggcgtgggaggatgatccggcggacccggaaaacgcgattgtggttctgtgcgacgatctgaaaaacaactttaagaaagagccgatccgtaacattctgcgttacatcttcaccattcgtcaagaatgcagcgcgcaggacatcctggcggcggcgaagtacaaccaacagctggatcgttataaaagccaaaaggcgaacccgagcgttctgggtaaccagggctttacctggaccaacgcggtgatcctgccggagaaggcgcagcgtaacgaccgtccgaacagcctggatctgcgtatttggctgtacctgaaactgcgtcacccggacggtcgttggaagaaacaccatatcccgttctacgatacccgtttcttccaagaaatttatgcggcgggcaacagcccggttgacacctgccagtttcgtaccccgcgtttcggttatcacctgccgaaactgaccgatcagaccgcgatccgtgttaacaagaaacatgtgaaagcggcgaagaccgaggcgcgtattcgtctggcgatccaacagggcaccctgccggtgagcaacctgaagatcaccgaaattagcgcgaccatcaacagcaaaggtcaagtgcgtattccggttaagtttgacgtgggtcgtcaaaaaggcaccctgcagatcggtgaccgtttctgcggctacgatcaaaaccagaccgcgagccacgcgtatagcctgtgggaagtggttaaagagggtcaataccataaagagctgggctgctttgttcgtttcatcagcagcggtgacatcgtgagcattaccgagaaccgtggcaaccaatttgatcagctgagctatgaaggtctggcgtacccgcaatatgcggactggcgtaagaaagcgagcaagttcgtgagcctgtggcagatcaccaagaaaaacaagaaaaaggaaatcgtgaccgttgaagcgaaagagaagtttgacgcgatctgcaagtaccagccgcgtctgtataaattcaacaaggagtacgcgtatctgctgcgtgatattgttcgtggcaaaagcctggtggaactgcaacagattcgtcaagagatctttcgtttcattgaacaggactgcggtgttacccgtctgggcagcctgagcctgagcaccctggaaaccgtgaaagcggttaagggtatcatttacagctattttagcaccgcgctgaacgcgagcaagaacaacccgatcagcgacgaacagcgtaaagagtttgatccggaactgttcgcgctgctggaaaagctggagctgattcgtacccgtaaaaagaaacaaaaagtggaacgtatcgcgaacagcctgattcagacctgcctggagaacaacatcaagttcattcgtggtgaaggcgacctgagcaccaccaacaacgcgaccaagaaaaaggcgaacagccgtagcatggattggttggcgcgtggtgtttttaacaaaatccgtcaactggcgccgatgcacaacattaccctgttcggttgcggcagcctgtacaccagccaccaggacccgctggtgcatcgtaacccggataaagcgatgaagtgccgttgggcggcgatcccggttaaggacattggcgattgggtgctgcgtaagctgagccaaaacctgcgtgcgaaaaacatcggcaccggcgagtactatcaccaaggtgttaaagagttcctgagccattatgaactgcaggacctggaggaagagctgctgaagtggcgtagcgatcgtaaaagcaacattccgtgctgggtgctgcagaaccgtctggcggagaagctgggcaacaaagaagcggtggtttacatcccggttcgtggtggccgtatttattttgcgacccacaaggtggcgaccggtgcggtgagcatcgttttcgaccaaaaacaagtgtgggtttgcaacgcggatcatgttgcggcggcgaacatcgcgctgaccgtgaagggtattggcgaacaaagcagcgacgaagagaacccggatggtagccgtatcaaactgcagctgaccagc

SEQ ID NO: 922 - protein, 1054 aa, Artificial Sequence
  Synthetic
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFDVGRQKGTLQIGDRFCGYDQNQTASHAYSLWEVVKEGQYHKELGCFVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGDWVLRKLSQNLRAKNIGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTVKGIGEQSSDEENPDGSRIKLQLTS

SEQ ID NO: 923 - protein, 1054 aa, Artificial Sequence
  Synthetic
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELGCFVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGRWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGEQSSDEENPDGSRIKLQLTS

SEQ ID NO: 924 - protein, 1054 aa, Artificial Sequence
  Synthetic
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELGCFVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGDWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGEQSSDEENPDGSRIKLQLTS

SEQ ID NO: 925 - protein, 1054 aa, Artificial Sequence
  Synthetic
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELGCFVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGDWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGEQSSDEENPDGGRIKLQLTS

SEQ ID NO: 926 - protein, 1054 aa, Artificial Sequence
  Synthetic
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELRCRVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGDWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGRQSSDEENPDGGRIKLQLTS

SEQ ID NO: 927 - protein, 1054 aa, Artificial Sequence
  Synthetic
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELRCRVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLATMHNITLFGCGSLYTSHQDPLVHRNPDKAMKCRWAAIPVKDIGDWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGRQSSDEENPDGGRIKLQLTS

SEQ ID NO: 928 - DNA, 32767 bp, Artificial Sequence
  Synthetic
aacgaactccatctgggatagcaataacctgtgaaaatgctcccccggctaatttgtatcaatgattatgaacaacatgctaaatcagtacttccaaagtctatatatgactattacaggtctggggcaaatgatgaagaaactttggctgataatattgcagcattttccaggtaagaaaatttattttttaaaatcatgttttaaaattacacaaagaccgtaccaaaataagatctcctagttttacgttggtggtgtgtaattatttgttcagatttgtgcttagtagagagggaaaagttcttggggctgtaagaaatcttgggcctttaaattgttaaaaaatattccaagcctgtgaatcttgaggaactgactgcaaaagccaaacctatgttacttcacttggaaatatgacaacaattaatttaactacatgtaaaaatagcgataaattcggatgacttttctttttcttagtatgacagtaaatgcttatgttcatggtgtaggaaacagcattaaatgccagataaccatcttatccggatgaaccagactggattgttggctcaaatgttttcttcctgctggcttttcgtgttatcattcattttgattactgttgtctaaactttcactttagatttcaatttgtctatgcagcattaatctttcaactttgctgtttcatctctccttcaaagcacttcatctctcttcccaaattagttttcctttgactttcatatttcaaagcacaagatggtgggtgacatggtttatgttttctgtttgtaataaaaacaagaaataaaatcatttcaaagggtttttttttatagcagttacaaaaatggtttatttgctggagcaagagaggagtgccttcactacactacactcagtctcatccatctaacattatggctgttagtaaaggcaatcggtattgtgggtactcattgatggtgataaagacaaaaaggcagaaaatatgcagggggagagaattagccttcctccctgatttcttctttagtctacaacaaaatcactcaaaatcagttttccatatttaaattaggagaaataaaattatcctggccaaggtggtctctggtaggcagcactgattcacccacaaatccatgtagaagactgaaaatggcaatggggtgaaggatacggcctctccccaaccctttcaagccttgactttgtctcaggttttgcctggaacccaaatgagctcaacaaatgccagggaagtcatgggaagggaagttgactgagagtagaggggcttaaaattctgcatcattatttactattttggactcatttaaaagtttctgctcttggaagatgccccttcttgggccgatattaactttgtccaccaaaatttgcctatgagtggtctcttgaaaacactttaacccaaataggttattacaaccaaggaaatttcagacccttgacagatttatagagttagtgtctcagcattgctagacctccaatgctcaagtgattatttatttcatttgtatacagctttccttacttcttaattccctttgtcgcatgctagctaattaactagagctaattaggagtctccatgagctacactgtgtactacatgctgaggacaaagcagtgagccagacaaagttcctgtccctaggaacttacattcccctggatgcatatcagcctccataatgctgttgggttgaattgatgcaaaatgggcccaaaatagttggccaagtggaggtctcagagaggatgcaaaggggcgccccaaagcagatggatcacctatgcaaccctttaaaatgtagaaactttgggagacatagaaggcttggtgacttctaagttatgaactggaaaagtgcctcatgccttatgtgaattacatggtattcaagtgagtattcccatcctatgtgtgtaccgagtaacttagggataggacacagataatgaaaatgaatttgcagtgtcaccttttccatgaaccttgatcattctcttttgttcagctttaaattaaaaaaaaaaatcaatcaactttctttggaggacagctgatgctattttattatcaactagttgagtttttattgcaatacattttgcaatgtgtcctcttttgctgtatgactcgctaggtgaaccttgattcctcacactgcatcatgtagctggtcacgtgaaactaagaatagaaattctgccagggttgtggagactttgggttgatggcatgaaggaaatcaacctgaaatttcacattctgattctaatgaaaagtgcaaaacaatcaaacctcagataacccattgtgatacaaagccagagtatttcaaacacatttatgaaatttatacacctccccatctcgcaagtacaacaaaaggtcattcaccgtgacagcttttatttctctgtactcagctctgataatcacattttggagttctggggacatggaccactcatgtgacccagcagttgcttggagatatttttgggtaagacttcagactaatattactgtggcagtagaaaaaaatgtttaaaaggacaagtaaatggaaccacccagaacaaaatttcttacggtggttataacaaaacagggtaaatgtcaacttgctacattttgcatggctggaattgattgggattaattcaacgaagaacagtaatttgtttctcttacacatttattcaaagtagccttctcaactatggtcttcacgttgttgtagcttttttttctgaaattatcaatgatggaagatgattaaacaatttcgacacttagaagccctcatgatttcagaaaaggaaactcttttctgctgcgttacctattgagactgaagatggcatcattttcttttaaataacagatgggtaaaagtgatgtcattctttcactttaatatttgagaagtgatatgaagttaccagtgacattgtgttctcataggcataaatgtcacaaaataatttatctagtatccacaataggtgaataaggtgtttttgctttatatattttaactgtttagagtaaaaaattaatgtggagaaaattggaatgcagtattataggattacacaacttacaaaacatgaatccactatgtccagttagtgtgattcagaaacagcatgcagttataaagctgggtgaggcatgggtgtcttccttcaacagggcagctactttgtgaggagtgtatatatcatttgatttttttataagttaaatttgaggcccctgttagatgtgagggtgggccaaaattcctgtgaacagattctccccgttaccccgcttcctttactctggcatctcattttctatcctttgaaaacggtttattattcaattggttcaactgtttgccagttgaaccaattctttttccaaagtggaggcccaggaaagcacagtccgagaatatagtgaggtgctattttatgtatgattgtgggaaatttacttaaatttggagtggggttgggcaaggcttggaaagctagtgagctatctgacatagttgttactactatttgaaaaatatcaaaacatggaggactctttagataacatgcctgttcccattccattgattttatctaattttacgtagcaattacgttttgtgcattggttgacaagcctctgtattatcctcagaacagaaaatactgtttaagggaaattaagagcccgcagttactaaagtgactgcgccaccaagtggacaagtgtaaagccactgtctggagatggaaggattcagctttgctttataaatgggaatttgacctttaaaaatgtcccttttggcacgcacgcgcgcgcgcgcgcgcgaacacacacacacacacacacacacacacacacacacacacacacggctgctgccctgcagatttgcttgttcttgtcataaagctttcattgtttctctagctctaagtaaatattaatgccttccaaggctggcatgccaatggctgctattaagatcgttttctctcattctaataacacacttagagatgattggtaataaaaactctcttcaaggcttctgcttctcccccttcaaaatggagatcaaagaatcatgctgtgagggtccgtcaagaagaaaagactttcagcaacagagcatgtggtgtggcataaaataatgacaattataatgttcaaaggaatagcatagaaatcacacagtaaaacttctttattatgcttttcagggactggatgtttttactttattatgtgaggaagggttagattacagacccttagctattccacaaagcaatagaaggcagaatttcttcttccgctacaggaagcacgcttcgattaagggctttttctttttcttctttttttttctttaagttactgcattactatatcatacttcactatatttactaaaaagtcatgctgtttctggaagtagagttacatctaggaaatactaggtgaatgctggttagatatgcatgtgtgcctaaacaacacgtttattatactcatgcatactagaaatagggctgtattttcttcaattttaatcagtactaatgagaataataaatcaaaacaaataggagagatatattttgccaggaggaaagagaactagttcttctgtaaattttactggtgaatttttggttgctggtttattggtaattttcattccaacacagaagaatcacagaaacattcatttaaaataattttccggagtcaaaaactttttaacacccaaatttcagtttttgtcaaataacatttttgagaaaagtgttaaattaaactaataaaaaaccttccctcatcattagactttaatgaatatggcatataactaaataattttgaagaaaccaaattataattttaaaagtaattgcctgaagctgctgtttatcacataaaaagaagacaaactagacatagcatatcttcttaaactctaatctaaactctatgcatttgtataccatcttgattttcaagattggggaagtgaaacgaaaactatgttcacacaagaacctgtacgtgaatgtttgtagtggctttatttagaatttccccccaaactgtaagtattcaaaatgtcttttagcttgggaatgactggacaaatgatagtacccctgtatgatggaatattattcatcaaccaaaaggaacaaactattgacacgtacaacaacatgagaaaatctctaatgcgttatgttaagtgaaagaagccaaactcaaaaggctacatactgaatgattttgtttacatgatattcttgcaaagcaaaattatcaggacaaagaaaaaatgcatcagtggttgtcaggggattgaactggggagagtttctctgcaaaagaaaatggggacttttttgggaatgattgaactttttctagatcttgattgtcatggcagttacaccactgtatgcatttgtcaaaattcacaaaactgcagactaaaatgagtgaatactattatgtattagttatactttaataaataattgcttgggaaattcattatcctctaattgttaactttctaaccaaacaaacagtaaaattgcctcttttccattagctttatgaagtcatttgcttgtttggaaaaaatccaattatattttttcttttaactaaaatgtaatgtcaaagttttggttatgattctgaaactctaaagccttttattttattttattttttaattctagatggaagctgtatccaaggatgctccggaatgttgctgaaacagatctgtcgacttctgttttaggacagagggtcagcatgccaatatgtgtgggggctacggccatgcagcgcatggctcatgtggacggcgagcttgccactgtgagaggtaggaggaagattgtcaccacagggacagaaggaggctaacgtttatcgacctccttctctgaatgcaccaagcaaatatgttccttgatgtttttacactcagaaacattaagctcatggactctatcatcaaaatacttgttcttgcatgtcctgctcctcttctttccagctgtgtgactgggcaagatatcctctctctgcattggtttccttggctgtaaaatagggacaaaaattgtacctgcctcattgggttatggtgagaattgaatgagttcaggtatacaaagttcatggcagagagtaggggctcagtaactgttggttatattatgggtattaatagtactgtctcaggaaatggatctctgacaggtagacttgcccaaagtcacagctaggtagttacagaattggaattcagccctgtggctaccttatctcaaaaccctcctgcttcccccaaaccaaagtggttctcacagccaaattgcaaatggagcaacgtggttggttgtgttttcttccgtggttttgggtcatgattcttttttatggatgagttatattcccaatagagcagttccagctgtcttaggagggagtgatgagaaaatcaaatatgatgtaaagaaatctcttattagggctaatttattaactttccagttctctagcaactgtgaacatttgaaaggctgtgcagagtaaaaaatctccccaaattgtgctccagaaactaatataaaagttggaaatgaattattttgatgctaagcagagcagaaaaagaacacgactatataatattttaaaacattttagttttaagaattaaggatcttgtgaattcacttcccttcttgaaatgtctgacataaaattctgtcagggatatcagaatggcacaatgaggttttgctggacagacttagcagcttccttaattctaggaccacatacaaataagtggctttggggcctcagccttttgtctatggtaatcctgaaacataagtagagagaagaaaaaaaaagggaaatactaaatgggtaaatatctatacaaaatcaagataataaaggccctttcaggcttgaaactataggcaacaaccttagaacaaaagaaaacaaatgaacatcaaaaaactaaaactttagtgctcttaaatctcaatgaaaataaaaagtaaatggtaaactgaaagaaatggaaaaaaaatatgagactgtgaagggttaatgtcctttccacgtaaaaagcccttatatttgaagaagaaaataatatattgctcaaagggaaaaagagaaataagtgaacaaaagatataaataggaaatttacaaatggagacataaaagtgaccaataaacatatgaaaaatattcaatttcattaataagcaaagacatggaaattatgaccatctattttattttccgtatatcgaatttttattttaagatcaggcagtatgattaggttagggagaaaatgtgcatttcaaacagtgttgagaaaagtataaagtggaataatcttcctagaaaataatctggcactgtatatcaaagctctaaaaatgtaaattccatgtgatgttaaaaattctcttctaggaattccaaggaaataattatgatttttgaggaaaaaaatcatttctgcaaggattttcatgcttcttatttttagcaggaaaataatttgaaaaaaatacccaaacatcttataattggagatagtttgcaaaaaatatgatgcataaaaatgacatcaaatttaaaaattatactataggaagagtgcaataatgtagaatgatattttaatttaaaattgtgagaaatcagttgcaaacaatagtcaggtcctaaaatacatttagtttcaaagatcacaatttacaaatgtttatttataagtgatgagattactcctgactttatactcttctgatttttggctcaaccttataaactcttctttgaattattttgtaaggaggaaatgataacaattagatttaaaagagtagagataaagggacaagggaccatgaagagaatggaaataaagaaaggaagcagagaaagcaaaaagcagagctcacttggtaaggcaccctggagccagcaaattatttttaccacatgtattagttccttctcacactgatttaaagatactcttcgagactgggtaatttattaaggaaagaggtttaactgactcacagttctacatggctggggaggcctcaggaaacttacaatcatggtggaaggcaaaggggaaggaacgaccttcttcccatggtggcaggagagagaagtgcaagcagggaaatgccagatacttataaaaccatctgatctcatgagaactcacccactatcatgagaacagcatgggggaaaccacccccatgatccaatcacctcccactaggtctttccctcaacacctgggattataattcaagatgagatttggatggagaaacaaagcctaaccataccaacacatattgctttatttgatatttgacaggtgtttctgtccctgttttgtgggcaagtagctaaagttccagagaaaacagtttttcatagctcgtcaatgacagacttattctccaagtcacatttgatggttccaagaccagtctttattcttggtggagttgggctgagaagaaagaggagaagaaagaagaaaagaaagcttccttagaaactatgatttgacagtgtaagtaggactatttcctccagaagtaaccataagaagatattaaatgcctattacagtcttatccccttagatttatttaacacttataaagcaattatcatgttccagacactattttaagtatattacgagtattatagcattgaaggctcagagcggcccaaataaatcgatcatattattaaacctattttacacaggagaaactgaggtacacgccaggtgaataaccttgcctagggatgcacaattcataagtgatagagatgggattcagacagaggtattctgtctccagaatctgggctcctcaccactttgcaagagctttaatttcagaaactcctatgaagtgtcatgaggagaagcccattatgatcccctagaagtaattatagttttaggagcatgcaaagcagacccctcaggaagataagttacacaatagacatttggataaggtggatccagcagaacaaagagagggtggtgacatcgagattgcagaggaattggagaaggcaatggaagtgtacacatgttgccctcaaaaacatagggtcctccattgggttcctatcagggcagcaacatcagagtttctattctgtatttatactagaaacctctctccagggtttctaagttttcacctatgttttaaagactatctataggttattagtctatttaatatttaggtgtatccagaaagctgatggtcatcagctcatagcaggtgttctttggctggtgtgtttatgttgtgggacagtgggttacttgcaaggaaaggatgaatggctggagtagatggtgcttgtgctctgcatgtattcccttcttacttcccatttccatcagacctaccactttttgcctgacattatctgttgcaacatgagcccatggataggtgtgtttgaagtaggggaatgggagagagggttccctagctaatgatgtacagcagtaggtggataaatacctcagctctctttgctcaggtaactgaagcattttctaatatggtcacccagtgttccttggaaggattgagtcccagttgccccctgaggttgcctgcccatgaacacaccctcttttattggcttccttcccattcttttctcacttccccattccttcaattcattgagattgtttccaaataagatgacttgctctcacatctctgtgtcatttttggcttcttgaagtatgcaaaccaggataatagctaactgaaggctatagatagccacaggcaaatttaagtaacagtgtaagaatattcatacttggcagagatttatttataaaaactcagaaaattcacatggaattatgaagttattattgtatttattccatcattcccagaaagaatatggaaatcctctcaagcaagccagtccttgggaatattgggaaatctatgcaatttgttgtggagtatttttttttttgttaccctcctaaatatctggccgctaagcattcctgtctccagggacttagaccctagcaaggaagagaagttggggccaggttcagaaaacgggttagttatcaatctccctggagaagtgtccccctcagcagggtcagtgagagtaagtgaaacccattggtgcccacaggcaatggtctggcctgagtaattagaatgggcctccagaaagttctgggaattgctatggtgccatagtctcattttccccgttgactctccagatttattcagagtccaacttcaagggcctttctgcccttcctctcacaactgtggaataataataatccaccttattaactgggaccgagaactgagctcgactcttatttttttgagacagagtcttgctctgtcaccagactggagtgcagtggcactatctcagctcactgcaacctctgcctcccaggttcaagcgattcccctgcctcagcctcctgggtagctaggactataggcacgcaccgcgacggctggctaattttttgtattttagtatagacagggtttcaccatgttggccaggatggtcttgatctcctgacctcatgatctgcctgccttggcctcccaaagtgctgggattacatgcgtgagccaccgcgccctgtctgaactctacttttttacactgctgcatgtttgtagagtgaccaatgaagctatacttttttcattttcaaaatgatgatgaatacaaggttatcaaataaaacacagagggcccattatgtttgaatttcagataaacaacaaatcataggtgtcctgtatgtttgctcaatctggcaaccctggatgaataagagctctcacctgaggatttcttgtgaggattcatgaaataaatgctagaaatgcttacacactatctttatttgcccctcagagcccaaagtctctgaaatctttatctttcacacacaaaaactcactttcagaaaagtatattccatttacatctagtggaaataaaaattgttctttttctttgtgaaaaatatttttattttaagctttatgcagaaacctcagggaaaaaaaggtacttttaggagccaggcttgtaatgtaaatgtccaaaaaagatgaaattgaaacaaacaaacaaacaaacaaacaaacaaacaaacaaaaaacagtgcaagctcctgtgtggagactgcagtgagtctgagattgcatgttccatcagaagggggcagccacatcttagctcttgatgacccaagggagcagggatgtggggttgccaaatcttccaaaattttaagaagccagaaatcttgatttctatgtacaatctcctggtttttaaatgtgggcaaataaatcaaaattccctaaaacactgtttggggcaacaatgtgtgggccaaagtaaatacttttgtgggctacaagtgtcccctaggctgtacatctgggacatctgatttatgtggaaatttaccgagaactagttttatttctgtggcaggtcattttcactttctaggattatgtttcttcattgataaagtgagctacttgagcaagaccagtggattgaatgccacgtcccaaggaggctggggttgtttccagggatcttacagaacttaggtgtgatactgagcatgagctacttgtgttgcattttggtgttcaaaagaaaagttctttaaatagttctgctggaaagacaaaaaaaaaaaaaagaaaaaactttcacaacaaaaatctccaaaaacaaaaacccagaaaactggcatagaagtggatgatctttgcaatttttttcagtatataaataaatgattttgatcccatttaaaattttatcaaatgcaaaaagaaacaattcaaagtatagagctaccttttcttactctactgaaatctacactttatgtcagccctggagggtttagacgcactttatgtcagcccacttctttcgactgcactatgtcagccttggagggtttagatgaggcagtgagcatttgaatgcttttaatttccatttttcaaagtacattcttggtctataggagaggaacaagatatgtaactatctctgactattgctaaaaacacaaacgtctttaataaatgttgcataaactcagaaagtgatacttcaaagtcttgtgaaaaatgatgatcaccagcatttatacagcaattagtatgtgccacgcaatttgactttattatttattcatctatctttaccaccatcttaaaatatgtgagtgcaaaaccctgagaaactttctccaactcctgtgggtgtggaaatcgaggcttagagaggttaatgctttgctcagattattaatcacttaggcagtgctacctataatatcctgctctgttactggtatttccaaacgtcattaactgtagcaagaatcctaaggcaagcactatgctatcatcttaaaatatttattgcaaacatcctatgttttattgttttatctttttaactttgagaagataaaataagccacagaagtgaaattaattgggaaatcattcgctttttgcaaaatttgggagcataaacaatgggtcatgaattacaatcaaacaaaagataaaattctaagaagtcttttaaagtggaaaaaaataactgaaaaatactgaatggagggcagtttttcatgcactgtgttacgaataaaaaatttgattcaatggattacttaatcaacattttaatagttgtaaatcttataatatttaagctgttttataagtgcctctacttataatggcacatccgtttgaaactctagcagatcatttttatttatttttttgaatttttttctttatattctttaaagaaggatacaaaattatttctatgaatatttaacatatggaaggaaatagcaataataaacataaatgctaacacatataaaataggtggtatcattaggctaaattttagtcttccaggataagtagaacatctctgacttctcaaatatccaattaataaaatgcttactataccatttggtgctttaagaacattgccatggaaacctctcaggttttatgcacagtagctataataaaattttccttcatctttcatggagctacttgagattttttttctccctttaaacatgagaaatcaaaaagaaagagaaaagaaggattaaatattcatttatccttttgcttctgacttgttatgtgggcaagtgccacatgagggagtgctgggacctcatatcaagaaaaattaaaacctacctaatgcgttccaggaatgttcagcatattagcaaattcttattaaactgtcaaaaaaaaaaaaagttttaaaagaaattccagcccctggatgcaattagaggctaccacactggatttgatgggccataaaaccattaaatctaaacactttctttttgagcctaaaaggccagaacattccaaagtgaagttttgggactcagctatgacttgaccacctattaagatgcaggtggaacagattgcagagtaacacaaagagccacacagaccccagatgactgcattagggtgtaggtgagagttttagctgttgaattttctggattttccaagattaagtgatcaaccttaacaatgagtgaaagaccattcaacaggaagaattgtcatttcctttgctctaaacccaaacgatgtattttttgaaagctttattgatttatatatttatgtgttgtgctaggcgaccgactagatatatgtttcagcatacctactaggaaaatatccccattattctcaattttacctaatccaggcaaagcactggacttgctttaaggaacatttttactctttctgaagtggagtgcctgtcatgtatcaggtgcaatgcttggactttacgttcttgtgattaatccttacaataggcctgtgaagtaattctcattctgtttgacagtagagaagaaggaagcccttgaccaaggtctagtgccagtaatggtggtgatggggtttgaacctaagtctgtttcactctaaagtgtaaccaaattttatgttttagacttgcttttctaacaataaaaagtcagtgacatgctctttctgtgtgtaagcactcacacacacacacacaaacacacatccgtattacatatgcttatatatgtattaaaagattatggacatttgatatatatacatatactaaaatgtataattcattgctaaagtattttcatataaatagtggcttcagtgttaaaatcactttgcaatgaaacaagattgttgattaaaacacctattaaaaaattagaatctagccatattaaagacagtcatcgaatggagtgatttctacgattttgcaccaaaatttaagctattgggtggctttcttgagagcatgagattgcttcttctcagaattattaatgtgcctgatgacattaaaatgtgacagtgaaaaaagtcagaggctcacatgtgtatcccaacactgaagttgttaaacactgggaggttggttgaagttgttgtgtgcaaactcaatactccttaaaaccattatttaaaggcctatcactgtgttatggtctccatatgatctgccatttatgccaggacttgacaattcagtaaaatgacagaataataacacaggaatcactgcagtagagctaatgttttagtctgttgcagagttctgccctagaaatacagtgaaaacaaggaagggagagctaagatgtccctgagactaattgttccttgaaaatattttcataagtaaaaaagaggtctagaggtgtagtggcagtgtgatcactcaagattatatagctccggattcgttcaatgggccatgatgaaagcacggcaacgattaaatctggtttcttggtctttcttggcagtgtttaaattggttcagttccataaattgtaaattaagatctgtttgacaacttttaagtatttcaagcataattgtagttgaaggtttgttcttttagatcactgacttcagaactttatttttctggttaatctcaattgtaattttagacattcataaaacaatgttgactgcgtctatgtgatggtagatcctctgtgaagacctttatgatggtagttcccctgtgaagataggatgacacactcaatggacattatggtgcacagttatacaaacacttcactatgacaggccctgagtttagaaccacacaactgcttggtacttggtcatcgcatattttccccattacgtaatgacttcctgtgcagatgacaaaatgcgttttctcaacaaaattattttcagtgcagctgttttgatgactaagttttgtaggagctttttaatcaaatgcacctaagaaaaccccaacactttaggccctttgaacatattacacttttttgcttcctctttcctctttttccttaaaaccataatttggaaatttgattctgccttcccataaaagagaattattttcaaagaaattatttgggtctaaattaacatgttacttaattgttctgcttgaatctaggtatatgattagtcccatatgaattgatgttccaaataatttactctcattgataactaatattttctatttccctctattgttttgtggtggtggtggtggctgtggatgaacatcattctcaaatatattataattcccttcctcatcaagcccagcatgataaacttcagttttgcctgatggttcatcatcttatttctgtgtgtaagattgttggatttgacattaaacatttggaaactattttataattgataacttgtgctttctcagctttgagtaagcgctctcttcttcatcttataccattttatttttatttattattcacttctgcttctgatctgagatctaggaagctggacaaatcccagataagcaagctaaacaaacaaacaacaacaacaacaacaacaacaacaacaacacaacccaaactaaaccaaaccaaaatcatgggataatggttaagtgtactgaggggccattatgcgaacacagtttaattccttggctttaaaactaataagagaagaatacataaacaaatgtggcaaatgtacctgtgaccctctccagagggtgccaggctagaagaaaggcagatttatcagcaaggcatggcgggccattggcaaaccatgggacaaccactaccaacttcactgccattgctccatatttccctcccgttttcaatgagccccaactttgctcaggacatcacacatattctactaatttggatgagtccttttgaaagaaaatatctacctcatggttctcaaagtatggtccttggaacatcagcgtcagcaggactctggagcttgttagaaatgcagatcttaggtcgcactacagacctactgagtcagaatctgaattttgttaacatacccatgtgattcctcaaagattgagaagccctgatcagagcctgggatgaaagttcctgttggttccaagccaaaggcatagttcaggtcttcacacatgacactattagatgtagatggatattgttcccttctgaagaccctcaaggtcttctgagagcctattaagttcagaatgactgcctgaaatgagtgagaagtcacaaggagactctagataattaagagatgtgttcacagtagtctttgataaaaacctgggacaggcaggcttagtatgcaggcccctaaaatttatgtacacaatggatttcctatttttgcttcttcacatccagattacctggatcagaaataaatgttttcattaagacttgatgtgacaaacaaacaaaacaaaactctgccaagctctagaagaacaattgcatttcccagccagagggagaacactgccagtttttgctgttttccaaagctgtttacctgtcctagctcatttaaatcactgtactttggagttccggattagcgtccccagaggtagctgcattcatacttgatgagttcttttaaatctcagccattgattgtaggttccatagtataggaaatttagccaaccctctattgaatggcagtttagaaaggtcgagctacacttaccttatgtcaggttattgcagacccttgtggcatttttccaccctaggacatgtgatttaactctaatagaaatctttattatgggtgggtctgagattaacttttattctataaaacagaaatcatgccactggccgtagcccattttttgagatggagtggggggaatggatgatagtaaacaaggatattaatctcatttatttttatatcattatatttatagttacattgcaaatggaagagtagagaaaccaaaaacttacactgggaactttacaatttttcttccaagtattactgattgatgtttggactatgcaagtgctgccagccccttagactcactctgcagctccccccatggaaatttgtgaacaggttagggtggggatagggaaaagcatgttcttgtttcacttcttggattatttgttccaggctctccaaagtaatgtgtaccttgggaatgcagaaattatctccttagatattctctccctatatatgtcctcacagggaattcttggaattggagaagattccactctcctttaggagctttctccataaaggtattgagcattggacactatatttgcaagggaaaagaggaatgggtctcttgagcatcaaaatcattgtagaagaatctccaaactgtttttcaaaatgtctgtactaacttacattcctgacatcaatgggttcccttttctccacaagggttcccttttctttgcatcttcaccaacacttgttatcattggtgtttttgataataaccattctaacagttggaggtgatacttcattatgattttaatttaaatttccctgataattagtgatactgagcttctttcatatatctattggccatttatatctcttcttttgagaaatgtctgttcagatcctttgccaattttttttcttttttcaacttttattttagaatcagggagccatgtgcaggtttgttacaaaggtatattgcatgatgctgaggtttggagtgcaaatgaatccatcacctaggtagtgaccacaattccaaacaggtagtttttttcagcccttttccccctcccaaccccactgttgtattccccagcatctattgttaccatttttttgaccatgtgtatccaatattcagcttccatttataagtgacaacatgtggtatttggtttttggttactacattaattcacttaggttattgatttccagctgcatccatgttggtgcaaaggacattattttgttattttttatggctacatagtattccatggtggatatgtaccacattttaaaaattcaatccaccattggtgggcacctggattgattccatgtctttgctattgtgaatagtgctgtgatgaacatgcaggtgcacgtgtctctttggtagaatgacttattgtcctttgggaatatacccagttagtgggattgctggatcgaatggtagaaaaactctcaggtctttgagaaatctccaaactgctctctatagtggcttatttaatttacattccctacagcagtgtatcagccttctcttttctccacagactcaccaacatagtattttttgactttttaacaaaagtaattctgactggtatgagatggtatatcattgtggttttgatttgcatttctttgatgattagagatgatgagcatcattttcatatatttatcagcctcttttatgcctttgtttgagaagtatctgcaaatgtcctttgcccactttttaatggggttatctgttttgtcatgttgatttgtttaagtttcttaaagattctggatattagacctttgttggatgcatagtatgcaaatattttctccaattttgtaggttgcctgtttactcctttgattgtttctcttgctgtcctttgcctattttttaattgggttatttgttttctggctattgagttgtttgagttccttatttttttttttggatattagcactcattagatatacactttacaaatattttctcccaatacctgtgttgtctcttgattctgttaattgttttctttgctgtgcagaaacattttagtttcacacaattccttaaaaaactaaaaatagaattgccatatgatccagaaattctacttctggatatttattgagaggaattgaaatcagcatgttgaagagatatctgcacttctatgttcgttatagcattattcataatagtcatgatatgccatcaacctaagtatccattgacagatgaatggataaagaatgaggtgtatgtacacaaaggaatactattcagcctttaaaaagtgggaaattctgtaacaacatggatagacagatactatatgatcttacttatatgtggaatctaaaaaggtaggtctcacagaaacagatcataaaaaggtggctaccagaggctgggagaggaaggaaaagaatgaggaaagtgacatattgatcaaagttgtacaaagtttcagtgcgactggagtaataggttttagtgatctattgtactgcatggtgtccacagttaatagtaatgtattgtatatcttaaaattactaaacgattaggtatttaatgttctccctacaaaaaaatggtaagttggtgtattagtccactttcacactgctataaggaactgcccgagactaagtaatttataaagaaaagaggtttactggctcacagttctgtatggctggggaggcctcaggaagcttacaatcatggtgaaagggaaagcaggtatgtcttacatggtggcaggtaagagatcctgtgtgtgaagtgaagggggaagagtcccttataaaaccatcagatctcgtgtgagctcactcactagcatgaaaacagcatggaggaaatcacccctatgatccaatcacctctttccctcaacacatggggattacagttccctgccttgatgcgtgggattaaaatttgagataagatttgggtggggacacaaagccaaaccttatcagttggtgaggtgatgaatatggtcattagcttgtctaaatttgtctgcaatgtatacatagatcaaaacatcactttgtaccccataaacatgtgcaattactattttccaattaaaaataaatataaataaattaaaaataattgcaaaggaaagctggctgtggagaagattaacaaataatgacattaagaaattcaggtccttggcaaaattagaaatacatacaaagctatccagaacttatttttccaaatgcattaggcgtcctctcaccttaccctttacaattgcatggcttcagagattacacagaaaacgttcagaaacattgccccagtagatgatcttgcaatgctatgaagtaggcagaacagctgtggctatagcaattgtgcagatagaacgtacttcatggatggcaagactgggactctaggacaggctttcaatccattctaccctgttgttgttctgaaatgaaagttttatctcccagtttatataggtagccttatctttgatgcttcaatacctgagacctggccagtgtcccttttagtgattgtatgtgtgtgtgtgtgtgtcatatgcaatttccttatagcaatggcacagtgtatcactgtttaattaaagaagagaaagaaatgccaaacatacgaataaagtctgaatatatctgtaacattaaaagtgtaggtgtctatctttgaagatatgtcttaaggacaatgaaagagtcagtgagtaagagaagagagtcctgggatttcatacaagatcagtgttacttgatggtgtaggctcctaggtatttcatctttaggatataccgtctattacaaaagccaagatttttagatttggatcaacattagggaacttcattctaggcaagagccaggttttgcctttatgttaatatgacctcagctgtgagctccattttgccaggcatcttaaaactgcaacacatatcattggaatcttccgttacagtctaatacatagccacacattgggagcaagaatgaaatccaacccctgtcctttgcaaaatgcaatgagacagtgtctgctttgggagcagggagtcagaatttcattgtggacaatggataaggtgagtaaaagggcttaaaacatttgtgctttcaagccataggctaggataacgatagtcagaactttttgatgaagtctgaccatgctacgccatttataaaattttgaagcttgtaagtattaccccaaaatgagcagtgtgaactcaaagggtttatcattgtctctcaggcaaaggtaatatttgaattatttagcaaaggactttgagcaattggaagagatactcagctgctggtctctagcgctctaacagggtggatgccccccgctctgccggcactgatgtttaagttgctggattatgaggaagtctggggattccttggggagaaaaggaagtgatgacatattgaagcacaacgacatattgaagagactcgggggctggggtgataaacttcagagccgtggctatttaccaattggagtgtaagtattttaatattttaacaaacataattgccattctggtatgtaccaacttcatctcagatctgtccttaagaaataggcaaattctttattgcctctctgaatggttcatataaattcccaggctcccttagctcattctaacataaaactgtattaaaaataatgaatgtaattcatcaataattttcctttgtcatagcaaatagtcacaagtggattgagatcagagtgatcactcatatttgttctggggagaagggagcctgctgttttgctcctgttttctcctaggactagtattttagcttcaaatgataataccttagcacagactctgatattcctcctacatgcaggagcattctcttggaataattttggggatgccaattcaaaatttcagccatgtatgatttacttattggaaaataatcactgagcagcaataactccagcagttacttgtatcaaggtagaatcaagaaatagatggtatggaccaaacttgcttctctctaaatatgcatacccaagtgatttgggtaaaatgtttgtgaagggcttacatttcctgcaagtcagatggtttaagagaagtagaaattatgtgtgttttgcagcattttggtaatctgtgtggagtgtctgtagatatttctcatgagttcaagggaatccttttgtggattttgatgttcctattggcagagctgctgcttgactacatgatgtctttgtattaactacaaaaacatgccctatcatctgagtgattttctctgccagacccctttgtgcatccacactctgcacctccagtgtacggaggaccttcccactggattctaagattccatgccttcccaatgcatggcagtgtctctcatgcacatggcaaacctactctcttggatgtcactgccctgaaatattgagggagtacatttatctaggcatggtaccagggagtcatttagacatgtagggagtctagaaagatcattgccctgggagagtgctcagccatgctgagttctcctactttgttgctcatttctgtgtgaccttaggtaacatcctcttcaggactttttttttttttttttttttgacagggagtctcattctgtcatccaggctggagtacagtggtgtgatctcagctcactgcaatctccgcctcctgggttcaagcaatcctagtgcttcagtcgcctgagtagctgggattacaggcatgcgccactacgcccagctaatatttgtattttcagtagagatagggttttatcatgttgatcaggctggtcttgaactcctgacctcaagggatctgcctaccttggcctcccaaagtgctgggattacagatgagagccaccaaccctggccaggacataatttatttcaggtgaattgattgttggaggattttgatccaagcaatcaatgtcccttggtgttcctttcaaacagcagtaagtgacctgaatttattttccacatttccaaatcttaatgaaaatcagacaatggtctatatgttcatttgtgttcttacttaataaaatgtgggttttagacaatattttgccagtcatgaattcctatagaaggaactctttgggagaacagactagtgatctatagacatgatgacctccaactcagatcttctgtagctaaccactgaccgggagaacatgtatgaaaaacatcttcaaaggcattgaaaaattaacatttatcaaaaacaaaatacattttatttcatttgaacttagacctttactatctaatggctatggtactatttaaatgtcaaagtgtgatctagcatcagcctaatctggttagaaatgcaaactcttgggccacatctcagacttactggaccagaagctctgtgggtgggacccagaaatctgtgtttcattcacatgccctccaggggattgtcctgctaaagtttgagaatcatggaagctttttaacctctcattatagctttataagcagcaactcactggattcctatcaacatcctgtgagtgtcatttggacaagtatatttatacccatttgatgcatggtaggcacacagatgagtcaaatgacttgaaggaatagagttttacataatatacttttatatatttatacttctaatatatttatactttataacagatttgactgttttatatattgcatataaacattatatcagtttctcctccactaaggctgactccaattttactccaattttactaccaatttttggaagaaagcctacctatcactcatgttctctcaagtaccctctaaaactattagttagatgactctatttaattttccatttatttgcccgtttcttgctacctttccccccaaaatgtaactgctaccttgctcaaaaggatgtgtctacttgggatatctagcacacacattttatgagattttaaaagacaacataaatggtaaactatatatttaatacaattttgaaagacaaaattttaaaattaaaaaggaagaaaaaaattaaactaaccccataattctcccacccatcattagcatgtagtttgtttagattcatctaccaataagtagaattgtacaaatttgatatcatgtaatacatgtcattttgtaaacttttttctttccttaatatatctatatatcataaacatttttctatgtctatattattttaaaattgtaatacccagagttctccagagaaacagaattaataggatctcccccttggagatttctcatctttttctctctcgatagatacagatagatacatacataagtctatctctctatctctatctttatctctaaaacacctatccatagatagacatttttaggaattggctcatgtgtttgtggaagcttgcaagttcaaatgtgcagagtaggtgggcaagctaccagggaaatgttgatgttgcagttccagtctgaaggcaggctccttgcagaattcttctttttcttagcagtcttacttccccttcctcttccccttctccttcccctttcccttcttcttctccttccccttcttcttattctccttcacagacttatttttaaggccttgagctgattagataagacccactcacattatgagggataatctgctttacctgtagtctactaattaaaatgttaatctcatctaaaaaacaccttcatagcagcattcagacatgtttcaccaaatatctgggcaccatggtttagcatattgatgcagaaaattgattatcataataatattatttttttttgagatgtagtttcactcttgtcacccaggctagagcgcaatgctgcaatctcagctcacttcaacctcttcctcctaggttcaagcgattctcctgcctcagcctcctgagtagctgggactacaggcgcccatgaccacgcccggttaattttgtgtttttttagtagagatggggtttcaccacgttggtcaggctggtctcgaactcctgacctcaggtgatccacccgcctcagcctcccaaagtgctgggattacaggcgtgagccactgtgcccagcaataatattaattttaatgggtgtgttcatttcattttatatatgacctacaatttaaccaatcccctaaggctggatgttcaggttcttaatattttttgcccgtatttacagacacctttgactattggatttattttgttcttcaggaacaatatacaaagtgtggaaagaaatgtatatttctaatcattggaaaataaacactgagcagaaataactccagtagccatttgtatcagaggaggtagaatcaggaaatagatggtatgggccagactttcttctctctttaagagatttgacttcatattgccaaattgcccttctagatgtctttactcatccaactacaattcaaaggtttgggagggtaagcaatgccaggcccatcttgatcatcccctttctttctcagcctgtcagtccctgggaacgggcatgatgttgagttcctgggccacctcctcaattgaagaagtggcggaagctggtcctgaggcacttcgttggctgcaactgtatatctacaaggaccgagaagtcaccaagaagctagtgcggcaggcagagaagatgggctacaaggccatatttgtgacagtggacacaccttacctgggcaaccgtctggatgatgtgcgtaacagattcaaactgccgccacaactcaggtaaccatgatcatgtgggccccgagctgaggcgaaagggatcttgactgggaatgttagggtctgggttctactgatagcaacgttgctaaacatctagttaatcttcagctaatcacatcccttttgtagacatcactttttttgagatacacaatagaaacagaaatggcctctataaaagtccaataaattttcagaccagagtgcattaagggctttggctttgggaagtatgaattgctatacagatggaagatactgaattttgcccaagcagcagtttattattatcatcctggtgccctatttctttgttaaagtcaaagagccacctttaccttttatttttaatggtacatgggacagctaaggctaagaagattgaagaaagaaaataatgaaggtttaaaaaagccacatctttgatccctcactgtctacttcttctttcagcaatattcctttcactgtggttcatccatgggtcaagattcattgattcattcactcaaatcattcatcttagcaaaaacaatatatcacataatctgatgttgaactataaaggtttcatcaggtcattcattcaccctgtccacaagctgtgaattattatctctttcctggttgtattttgggattacaatcatcttgagtcaaagctggaaactgagtggaagtctctgggaaagactcaaacctccttaagctatacacctcttttccccatcagattttccttccttcagtttccaccaaaatgtgctcttggatttttcatatgaatgtataatgtacctcaggcctataagtattttaaaagggatcaaaatcttagttttaatggaggacatttttatgatggactcctacagcatccatcagaatatgtaagatgatgaggaatgtcttcctgtgttcccagatctcatgccacagaggcccttgcttactctatgtttgaattgtatttggaaaaaaaaaaaaaaacaaaaaactagggctagcaaaattgaaaaaagataaaagacgaaagaagccacatgtaaacatactgtgtttactcttctaaaatattaaaaaatgaaaagatccaaaatcaaattaatattcccctggaatttcatatctatttcagtgactgtggagtgaatctcaccacgaaagttgctgcagtcttgtataagtttcacatagttttactgtgtttgtgcctatgtgagaataaactactgtgcataaaatcttgctgttgagccatgtgtgaattagctgtgtgatgttacctccctgttactaccaggctggtttaggatatcatttctgtatgtggcaccaggattagaccaatgacagaaaaagaaagtgctctccctgccaaactggccaataaaactgttccacatatcccagactcagggttacctaaacaacctgtgtttaaagagaacaaaaacaaaagcctctgacatagtcttactccttgccaaattcgtcagaaagctgatggattcaaattcccccaatatgaatcccgtatttacattatttctctattttgactacttttttttttttttaaagactttctaaatagtttcccactatcgaggcttcttagaggaaacatttctcattatttccccttggctatttgaaaaggaatttgttcttccttttcctccatctcttaacactactactactaacaatagtaacaacaatagtaagtacagtagggttttttgttttgtttttaacttaagacatactttcttgttctggataccaaaatatgtttcacagaggcatctacttagatggggtgcagatgacacagttgttaattctggcaggtacctcttgcttcttcactgctggggctactcagtgagtggcaggaaggttgatttgctttccccccttttcttttgctcctgggctccttcccagatgatgtgacgggccatgaaacaaagactcttttcagctgtcggtgtgcatagaactggctgcggcttcctagcttgtcacatctccggtctgaagatgatcaaataatgagcaacacatccaggttatagggaacacgggaaacaccccgcagctgggtgtaccccagcccctcagagtgcacattggtgttgtttgtcctagtggacttcggagtaggccagtgccttctggtcagttcctcagtggcccacattcagctcttaaaggcagagcatgctaacgggaggtccaggcttccgcctgaggccaaatacaccccaaaagctcatctgttatagcctgatatgaaatcggtttctttctgcaactgacctgactcatagaaagtgaagcctggcttttcataagtgaagtttggcaggcaagggaggcaggaaatccagaggagaatgagcctgtaaagcatggctccttccagcccttgttacttcctctgcccaagtgtgggggagggtcctgtctcttggcatctgggcccagcaagagttcagaggtttggtagtctctgcttggtccatatgcaaaacacatgtatgtgtatacattattaatggcaagggggttcctgaaactgagagggagtaaggagacttctcatctgctcttggaagaagcaaggaatgaagccagttcagtagactgattcctgaggctttggggcaggaactttttctttctccatatccccatggagatggttcatttaccctgaattaagatttggcccttcggtgcagtgccaaggcagtttaaagagaagaaaagtaatttctgatcattgactaagatcaaggtaaatcatgacacttatcctttctatgatttgccagtgacatgttttcttaagcccagaaatgatttattgatcgcagcagccagaatatatcacactaaaacagatcagcctgccactgtcttctcaggtctctcatgattaaagtggcctgctttaaagtagactcaatgtgaataggtctccatgacctctgcctcactgcgtagcactcacatcctcacccactcttgcactctggcttccctgcggttctttcaatatgccaggcatgctggaaccccggagcctttgcactggctgttccctctgtctgtaacagtcattcgcagaatcaacgcatgactaatagcctcacttccattgagtcttgacattaggaatggatatacatgtctatattgggaaaccacaataaaaattgatggtagagatgcaaatatgagcaagataacagggtgggggcaggggagagaggtcagtggaggacttggcacagtagcctcttaaatggcacaatagcctcttaaatttttggttaagaaatcattcacattgataagtatggcaggataaaggtgtccatgagtgaatcccgggaacctgttactttatgtggcaaaagggactttgcagatgtgattaacttaaggggcttgagatgggaagatttttcctgtttttatcagtaggcttgatataattaaaagggtccttataagagggaagcaagagtgtcagagtcagagaaagagatgaaatgacagatgtagaggttggaatgatgtggtcaggaaccagggaaagcagggggtatctagaagctggaaaagacaagggaatagggcttcccctagattctccagaaatacagccctattgatatcttgagtttagtccagtgagacttattttagacttctgacatttggaactgtaatataataccttcatgttatttttattgctgttgtaacaaataaccacaaacacagttctactaatttctttctcaaggtagcttctcaattttgccaacgctggttaccatacatacttaaagtttcattttgagtctctgaaaactcacatctctcttaatctgctctacttttttcttggctttgtatagtgcttatgttctgctacactttgtaatttattgattatgcttaccatgggcagggattcttaactgttttatttatttatatatcttaaacattgaaaacactggcatgtagtagatgcttaataagtaattgttgactcaatcgataaaatatactagaacatacaagattttcccaatgtaacataactagtaagaggctgaaccgggatttgaactcaaaattcattccctgaaccttcttctagcagccacattgaggaagaaattaccagggctgtgttctcaacacaagtgttttccgaaccacagaattaaaggctggtggcccatgtatcagtgtctgtatttatgagcccctctttcaatctctttcttttcatattgtgttgatgctgtagcttctactggtcatgttatttttttgtttcccaagacggaattatgtggctttatctttaatgttgcattatcaatacttataataaataatattatgtattactcaatattcatgattaatagtgttactattggttatttaataatgtttaacttacattagcagttgttactatttttatgatgctaaattactaacagctaaaacaacttctatattaaaaagtatatttgagtgccactcaagagataatgagtaccttacaaagaagaaatcttgtttctcacctttgcgtcattaaacagatcaggatttggagaattaagccctaagtaatagtgttattattttgatctcacccctttttttcttatgaaatggaatactttggttatcagaagccactttaagcatatatatatatatatatatatatacatatatatatatatatatgtcataatccgaataaaaatagcattcatggaggtttcttttggagcctttggtaaaacactccatcgtgggtctctgtcaagatatctgaaaactttttcttggcttctggctttgaacaaagtttcagagtaacaacaaggcttcattgtgcactgaaatttctgtaaggcaacattcattcaagtgttgattcgcatttcaccatccaagaataacaacagttatttatataattttatccacgtttctgttttttcctatccatttcaccctttcaccccacccctgctgaaacactggagcttgtttgggatgggggtggggtgccatgcagactacatacacatacagatgtttttctttttcttttcccggtcttgctatgggatagacagactggactttttcttattaacaatattatttaaaagcttggaatttattatcatttaatcatttgtatgtaatgaaataggtctccatggtaaagatgtgtttattgaccagcggttagctttattcaaattagggtgaccatagaagaccaaggactatgatataatgtacaatcctaagtggtttgatttaaataaaaagaaagaccaggcatttcagctaaaatccccaccaaagcccaatgactagatgggcatccatatgactcaatgaaattttctatgatcttaaatggccatctgagtccgtgaaactataggactaactattcaatccttattgagaaagccttgttaatagcttgaattgagttatatgggataggaatgttcatatctttatgacaatatatgccacctaagctacataaccagctgtgttagctaaaatactctaaagtgtaaaaaatcatagttttctattaaaggaagtcatgattgttaaaaataatttttaaatagtgtgcctagattcttctagtataatatataattttttttttttttttattttgagacagagtcttgctctgtcacccaggctggagtgcagtggctggagttgctcactgcaacctcgcctcccgggttcaagcgattctcgtgcctcagcctcccaagtagctgagattacacgtgcccaccactatgtccggctaatttttttgaatttttagtagagactgggtttcatcatgctggccagactggtcttgaactcctgacctcaggtgatctgcccacctcggcctcccaaagtgctgggattacaggcatgagccattgcgcccagccgatatataaatttttatatggctccatgatcttctctacatttaatgacagaactggtggaggggaagaaagagatgggactaagccagagatcaatatacatacaactatactttgaccaaaaaaagggagattgactggcaggggaattaatagtatgcagaagagcaaggtgagtccagtcactgtcattattcaaaaacagcctttcaggagaagtttgcaactgaatttgggactgtgggcagataagtcacaggaatgattctattgtgtatcctgaagtcatccatccagctaggagtcagaggtgcaggctgaaaagacattgcccctagagtggggaactgccaaaatctagccaggatattaggccaagagaaaagacctcaggcacaggggaagccagcttcaga

SEQ ID NO: 929 - DNA, 223 bp, Artificial Sequence
  Synthetic
aacgaactccatctgggatagcaataacctgtgaaaatgctcccccggctaatttgtatcaatgattatgaacaacatgctaaatcagtacttccaaagtctatatatgactattacaggtctggggcaaatgatgaagaaactttggctgataatattgcagcattttccaggtaagaaaatttattttttaaaatcatgttttaaaattacacaaagaccg

SEQ ID NO: 930 - DNA, 252 bp, Artificial Sequence
  Synthetic
tgattctgaaactctaaagccttttattttattttattttttaattctagatggaagctgtatccaaggatgctccggaatgttgctgaaacagatctgtcgacttctgttttaggacagagggtcagcatgccaatatgtgtgggggctacggccatgcagcgcatggctcatgtggacggcgagcttgccactgtgagaggtaggaggaagattgtcaccacagggacagaaggaggctaacgtttatcg

SEQ ID NO: 931 - DNA, 356 bp, Artificial Sequence
  Synthetic
ggagggtaagcaatgccaggcccatcttgatcatcccctttctttctcagcctgtcagtccctgggaacgggcatgatgttgagttcctgggccacctcctcaattgaagaagtggcggaagctggtcctgaggcacttcgttggctgcaactgtatatctacaaggaccgagaagtcaccaagaagctagtgcggcaggcagagaagatgggctacaaggccatatttgtgacagtggacacaccttacctgggcaaccgtctggatgatgtgcgtaacagattcaaactgccgccacaactcaggtaaccatgatcatgtgggccccgagctgaggcgaaagggatcttgactg

SEQ ID NO: 932 - DNA, 276 bp, Artificial Sequence
  Synthetic
acgtatttctaatttggcaaatttctcattttatgcatttcttattttaggatgaaaaattttgaaaccagtactttatcattttctcctgaggaaaattttggagacgacagtggacttgctgcatatgtggctaaagcaatagacccatctatcagctgggaagatatcaaatggctgagaagactgacatcattgccaattgttgcaaagggcattttgagaggttcgtttatttctctacttgaattcatactgactttgtgatcctttgtg

SEQ ID NO: 933 - DNA, 192 bp, Artificial Sequence
  Synthetic
ctgcctgttaagttacagtttccctaaggtgcttgttttactctctccaggtgatgatgccagggaggctgttaaacatggcttgaatgggatcttggtgtcgaatcatggggctcgacaactcgatggggtgccagccactgtgagttttggcagacgctaagatttccttttggagttcccatttccatc

SEQ ID NO: 934 - DNA, 444 bp, Artificial Sequence
  Synthetic
taacaattcagtgttaatagagtcacattattgaacttttctttccccagattgatgttctgccagaaattgtggaggctgtggaagggaaggtggaagtcttcctggacgggggtgtgcggaaaggcactgatgttctgaaagctctggctcttggcgccaaggctgtgtttgtggggagaccaatcgtttggggcttagctttccaggtaactggacaaagaaatgaatatataaaatagacaacttgacagtaaaacaaatgaataaaacaagtcagactgatttagttctgaatcactctgtatcttttcacttggttagggggagaaaggtgttcaagatgtcctcgagatactaaaggaagaattccggttggccatggctctgagtggtaagactcattcttgtttacaactttcttttcttttatgatctttaagt

SEQ ID NO: 935 - DNA, 729 bp, Artificial Sequence
  Synthetic
tgattattattgcattcagttcatattaaatgtatgcattattttttcagggtgccagaatgtgaaagtcatcgacaagacattggtgaggaaaaatcctttggccgtttccaagatctgacagtgcacaatattttcccatctgtattattttttttcagcatgtattacttgacaaagagacactgtgcagagggtgaccacagtctgtaattccccacttcaatacaaagggtgtcgttcttttccaacaaaatagcaatcccttttatttcattgcttttgacttttcaatgggtgtcctaggaaccttttagaaagaaatggactttcatcctggaaatatattaactgttaaaaagaaaacattgaaaatgtgtttagacaacgtcatcccctggcaggctaaagtgctgtatcctttagtaaaattggaggtagcaaacactaaggtgaaaagataatgatctcattgtttattaacctgtattctgtttacatgtctttaaaacagtggttcttaaattgtaagctcaggttcaaagtgttggtaatgcctgattcacaactttgagaaggtagcactggagagaattggaatgggtggcggtaattggtgatacttctttgaatgtagatttccaatcacatctttagtgtctgaatatatccaaatgttttaggatgtatgttacttcttagagagaaataaagcatttttgggaagaa

SEQ ID NO: 936 - RNA, 36 nt, Artificial Sequence
  Synthetic
ucucaacgauagucagacauguguccucagugacac

SEQ ID NO: 937 - RNA, 36 nt, Artificial Sequence
  Synthetic
uuuuaacaacacucaggcauguguccacagugacac

SEQ ID NO: 938 - RNA, 36 nt, Artificial Sequence
  Synthetic
uugaacggauacucagacauguguuuccagugacac

SEQ ID NO: 939 - RNA, 36 nt, Artificial Sequence
  Synthetic
ugcccucaauagucagauguguguccacagugacac

SEQ ID NO: 940 - RNA, 36 nt, Artificial Sequence
  Synthetic
ucucaaugauacuuagauacguguccucagugacac

SEQ ID NO: 941 - RNA, 36 nt, Artificial Sequence
  Synthetic
ucucaaugauacucagacauguguccccagugacac

SEQ ID NO: 942 - RNA, 36 nt, Artificial Sequence
  Synthetic
ucucaaugauacuaagacauguguccucagugacac

SEQ ID NO: 943 - RNA, 36 nt, Artificial Sequence
  Synthetic
ucucaacuauacucagacauguguccucagugacac

SEQ ID NO: 944 - RNA, 36 nt, Artificial Sequence
  Synthetic
ucucaacgauacucagacauguguccucagugacac

SEQ ID NO: 945 - RNA, 36 nt, Artificial Sequence
  Synthetic
ucucaacgauacuaagauauguguccucagcgacac

SEQ ID NO: 946 - RNA, 36 nt, Artificial Sequence
  Synthetic
ucucaacgauacuaagauauguguccccagugacac

SEQ ID NO: 947 - RNA, 36 nt, Artificial Sequence
  Synthetic
ucucaacgauacuaagauauguguccacagugacac

SEQ ID NO: 948 - RNA, 36 nt, Artificial Sequence
  Synthetic
ucucaacaauacucagacauguguccccagugacac

SEQ ID NO: 949 - RNA, 36 nt, Artificial Sequence
  Synthetic
ucucaacaauacuaaggcauguguccccagugaccc

SEQ ID NO: 950 - RNA, 36 nt, Artificial Sequence
  Synthetic
ucucaaagauacucagacacguguccccagugacac

SEQ ID NO: 951 - RNA, 36 nt, Artificial Sequence
  Synthetic
ucucaaaaauacucagacauguguccucagugacac

SEQ ID NO: 952 - RNA, 36 nt, Artificial Sequence
  Synthetic
gcgaaacaacagucagacauguguccccagugacac

SEQ ID NO: 953 - RNA, 36 nt, Artificial Sequence
  Synthetic
ccucaacgauauuaagacauguguccgcagugacac

SEQ ID NO: 954 - RNA, 22 nt, Artificial Sequence
  Synthetic
agacauguguccucagugacac

SEQ ID NO: 955 - DNA, 3222 bp, Artificial Sequence
  Synthetic
atggcttccatctctaggccatacggcaccaagctgcgaccggacgcacggaagaaggagatgctcgataagttctttaatacactgactaagggtcagcgcgtgttcgcagacctggccctgtgcatctatggctccctgaccctggagatggccaagtctctggagccagaaagtgattcagaactggtgtgcgctattgggtggtttcggctggtggacaagaccatctggtccaaggatggcatcaagcaggagaatctggtgaaacagtacgaagcctattccggaaaggaggcttctgaagtggtcaaaacatacctgaacagccccagctccgacaagtacgtgtggatcgattgcaggcagaaattcctgaggtttcagcgcgagctcggcactcgcaacctgtccgaggacttcgaatgtatgctctttgaacagtacattagactgaccaagggcgagatcgaagggtatgccgctatttcaaatatgttcggaaacggcgagaaggaagaccggagcaagaaaagaatgtacgctacacggatgaaagattggctggaggcaaacgaaaatatcacttgggagcagtatagagaggccctgaagaaccagctgaatgctaaaaacctggagcaggttgtggccaattacaaggggaacgctggcggggcagaccccttctttaagtatagcttctccaaagagggaatggtgagcaagaaagaacatgcacagcagctcgacaagttcaaaaccgtcctgaagaacaaagcccgggacctgaattttccaaacaaggagaagctgaagcagtacctggaggccgaaatcggcattccggtcgacgctaacgtgtactcccagatgttctctaacggggtgagtgaggtccagcctaagaccacacggaatatgtcttttagtaacgagaaactggatctgctcactgaactgaaggacctgaacaagggcgatgggttcgagtacgccagagaagtgctgaacgggttctttgactccgagctccacactaccgaggataagtttaatatcacctctaggtacctgggaggcgacaaatcaaaccgcctgagcaaactctataagatctggaagaaagagggtgtggactgcgaggaaggcattcagcagttctgtgaagccgtcaaagataagatgggccagatccccattcgaaatgtgctgaagtacctgtggcagttccgggagacagtcagtgccgaggattttgaagcagccgctaaggctaaccatctggaggaaaagatcagccgggtgaaagcccacccaatcgtgattagcaataggtactgggcttttgggacttccgcactggtgggaaacattatgcccgcagacaagaggcatcagggagagtatgccggtcagaatttcaaaatgtggctggaggctgaactgcactacgatggcaagaaagcaaagcaccatctgcctttttataacgcccgcttctttgaggaagtgtactgctatcacccctctgtcgccgagatcactcctttcaaaaccaagcagtttggctgtgaaatcgggaaggacattccagattacgtgagcgtcgctctgaaggacaatccgtataagaaagcaaccaaacgaatcctgcgtgcaatctacaatcccgtcgccaacacaactggcgttgataagaccacaaactgcagcttcatgatcaaacgcgagaatgacgaatataagctggtcatcaaccgaaaaatttccgtggatcggcctaagagaatcgaagtgggcaggacaattatggggtacgaccgcaatcagacagctagcgatacttattggattggccggctggtgccacctggaacccggggcgcataccgcatcggagagtggagcgtccagtatattaagtccgggcctgtcctgtctagtactcagggagttaacaattccactaccgaccagctggtgtacaacggcatgccatcaagctccgagcggttcaaggcctggaagaaagccagaatggcttttatccgaaaactcattcgtcagctgaatgacgagggactggaatctaagggtcaggattatatccccgagaacccttctagtttcgatgtgcggggcgaaaccctgtacgtctttaacagtaattatctgaaggccctggtgagcaaacacagaaaggccaagaaacctgttgaggggatcctggacgagattgaagcctggacatctaaagacaaggattcatgcagcctgatgcggctgagcagcctgagcgatgcttccatgcagggaatcgccagcctgaagagtctgattaacagctacttcaacaagaatggctgtaaaaccatcgaggacaaagaaaagtttaatcccgtgctgtatgccaagctggttgaggtggaacagcggagaacaaacaagcggtctgagaaagtgggaagaatcgcaggtagtctggagcagctggccctgctgaacggggttgaggtggtcatcggcgaagctgacctgggggaggtcgaaaaaggaaagagtaagaaacagaattcacggaacatggattggtgcgcaaagcaggtggcacagcggctggagtacaaactggccttccatggaatcggttactttggagtgaaccccatgtataccagccaccaggaccctttcgaacataggcgcgtggctgatcacatcgtcatgcgagcacgttttgaggaagtcaacgtggagaacattgccgaatggcacgtgcgaaatttctcaaactacctgcgtgcagacagcggcactgggctgtactataagcaggccaccatggacttcctgaaacattacggtctggaggaacacgctgagggcctggaaaataagaaaatcaagttctatgactttagaaagatcctggaggataaaaacctgacaagcgtgatcattccaaagaggggcgggcgcatctacatggccaccaacccagtgacatccgactctaccccgattacatacgccggcaagacttataataggtgtaacgctgatgaggtggcagccgctaatatcgttatttctgtgctggctccccgcagtaagaaaaacgaggaacaggacgatatccctctgattaccaagaaagccgagagtaagtcaccaccgaaagaccggaagagatcaaaaacaagccagctgcctcagaaa

SEQ ID NO: 956 - protein, 1074 aa, Artificial Sequence
  Synthetic
MASISRPYGTKLRPDARKKEMLDKFFNTLTKGQRVFADLALCIYGSLTLEMAKSLEPESDSELVCAIGWFRLVDKTIWSKDGIKQENLVKQYEAYSGKEASEVVKTYLNSPSSDKYVWIDCRQKFLRFQRELGTRNLSEDFECMLFEQYIRLTKGEIEGYAAISNMFGNGEKEDRSKKRMYATRMKDWLEANENITWEQYREALKNQLNAKNLEQVVANYKGNAGGADPFFKYSFSKEGMVSKKEHAQQLDKFKTVLKNKARDLNFPNKEKLKQYLEAEIGIPVDANVYSQMFSNGVSEVQPKTTRNMSFSNEKLDLLTELKDLNKGDGFEYAREVLNGFFDSELHTTEDKFNITSRYLGGDKSNRLSKLYKIWKKEGVDCEEGIQQFCEAVKDKMGQIPIRNVLKYLWQFRETVSAEDFEAAAKANHLEEKISRVKAHPIVISNRYWAFGTSALVGNIMPADKRHQGEYAGQNFKMWLEAELHYDGKKAKHHLPFYNARFFEEVYCYHPSVAEITPFKTKQFGCEIGKDIPDYVSVALKDNPYKKATKRILRAIYNPVANTTGVDKTTNCSFMIKRENDEYKLVINRKISVDRPKRIEVGRTIMGYDRNQTASDTYWIGRLVPPGTRGAYRIGEWSVQYIKSGPVLSSTQGVNNSTTDQLVYNGMPSSSERFKAWKKARMAFIRKLIRQLNDEGLESKGQDYIPENPSSFDVRGETLYVFNSNYLKALVSKHRKAKKPVEGILDEIEAWTSKDKDSCSLMRLSSLSDASMQGIASLKSLINSYFNKNGCKTIEDKEKFNPVLYAKLVEVEQRRTNKRSEKVGRIAGSLEQLALLNGVEVVIGEADLGEVEKGKSKKQNSRNMDWCAKQVAQRLEYKLAFHGIGYFGVNPMYTSHQDPFEHRRVADHIVMRARFEEVNVENIAEWHVRNFSNYLRADSGTGLYYKQATMDFLKHYGLEEHAEGLENKKIKFYDFRKILEDKNLTSVIIPKRGGRIYMATNPVTSDSTPITYAGKTYNRCNADEVAAANIVISVLAPRSKKNEEQDDIPLITKKAESKSPPKDRKRSKTSQLPQK

SEQ ID NO: 957 - protein, 1074 aa, Artificial Sequence
  Synthetic
MASISRPYGTKLRPDARKKEMLDKFFNTLTKGQRVFADLALCIYGSLTLEMAKSLEPESDSELVCAIGWFRLVDKTIWSKDGIKQENLVKQYEAYSGKEASEVVKTYLNSPSSDKYVWIDCRQKFLRFQRELGTRNLSEDFECMLFEQYIRLTKGEIEGYAAISNMFGNGEKEDRSKKRMYATRMKDWLEANENITWEQYREALKNQLNAKNLEQVVANYKGNAGGADPFFKYSFSKEGMVSKKEHAQQLDKFKTVLKNKARDLNFPNKEKLKQYLEAEIGIPVDANVYSQMFSNGVSEVQPKTTRNMSFSNEKLDLLTELKDLNKGDGFEYAREVLNGFFDSELHTTEDKFNITSRYLGGDKSNRLSKLYKIWKKEGVDCEEGIQQFCEAVKDKMGQIPIRNVLKYLWQFRETVSAEDFEAAAKANHLEEKISRVKAHPIVISNRYWAFGTSALVGNIMPADKRHQGEYAGQNFKMWLEAELHYDGKKAKHHLPFYNARFFEEVYCYHPSVAEITPFKTKQFGCEIGKDIPDYVSVALKDNPYKKATKRILRAIYNPVANTTGVDKTTNCSFMIKRENDEYKLVINRKISRDRPKRIEVGRTIMGYDRNQTASDTYWIGRLVPPGTRGAYRIGEWSVQYIKSGPVLSSTQGVNNSTTDQLVYNGMPSSSERFKAWKKARMAFIRKLIRQLNDEGLESKGQDYIPENPSSFDVRGETLYVFNSNYLKALVSKHRKAKKPVEGILDEIEAWTSKDKDSCSLMRLSSLSDASMQGIASLKSLINSYFNKNGCKTIEDKEKFNPVLYAKLVEVEQRRTNKRSEKVGRIAGSLEQLALLNGVEVVIGEADLGEVEKGKSKKQNSRNMDWCAKQVAQRLEYKLAFHGIGYFGVNPMYTSHQDPFEHRRVADHIVMRARFEEVNVENIAEWHVRNFSNYLRADSGTGLYYKQATMDFLKHYGLEEHAEGLENKKIKFYDFRKILEDKNLTSVIIPKRGGRIYMATNPVTSDSTPITYAGKTYNRCNADEVAAANIVISVLAPRSKKNREQDDIPLITKKAESKSPPKDRKRSKTSQLPQK

SEQ ID NO: 958 - protein, 1074 aa, Artificial Sequence
  Synthetic
MASISRPYGTKLRPDARKKEMLDKFFNTLTKGQRVFADLALCIYGSLTLEMAKSLEPESDSELVCAIGWFRLVDKTIWSKDGIKQENLVKQYEAYSGKEASEVVKTYLNSPSSDKYVWIDCRQKFLRFQRELGTRNLSEDFECMLFEQYIRLTKGEIEGYAAISNMFGNGEKEDRSKKRMYATRMKDWLEANENITWEQYREALKNQLNAKNLEQVVANYKGNAGGADPFFKYSFSKEGMVSKKEHAQQLDKFKTVLKNKARDLNFPNKEKLKQYLEAEIGIPVDANVYSQMFSNGVSEVQPKTTRNMSFSNEKLDLLTELKDLNKGDGFEYAREVLNGFFDSELHTTEDKFNITSRYLGGDKSNRLSKLYKIWKKEGVDCEEGIQQFCEAVKDKMGQIPIRNVLKYLWQFRETVSAEDFEAAAKANHLEEKISRVKAHPIVISNRYWAFGTSALVGNIMPADKRHQGEYAGQNFKMWLRAELHYDGKKAKHHLPFYNARFFEEVYCYHPSVAEITPFKTKQFGCEIGKDIPDYVSVALKDNPYKKATKRILRAIYNPVANTTRVDKTTNCSFMIKRENDEYKLVINRKISRDRPKRIEVGRTIMGYDRNQTASDTYWIGRLVPPGTRGAYRIGEWSVQYIKSGPVLSSTQGVNNSTTDQLVYNGMPSSSERFKAWKKARMAFIRKLIRQLNDEGLESKGQDYIPENPSSFDVRGETLYVFNSNYLKALVSKHRKAKKPVEGILDEIEAWTSKDKDSCSLMRLSSLSDASMQGIASLKSLINSYFNKNGCKTIEDKEKFNPVLYAKLVEVEQRRTNKRSEKVGRIAGSLEQLALLNGVEVVIGEADLGEVEKGKSKKQNSRNMDWCAKQVAQRLEYKLAFHGIGYFGVNPMYTSHQDPFEHRRVADHIVMRARFEEVNVENIAEWHVRNFSNYLRADSGTGLYYKQATMDFLKHYGLEEHAEGLENKKIKFYDFRKILEDKNLTSVIIPKRGGRIYMATNPVTSDSTPITYAGKTYNRCNADEVAAANIVISVLAPRSKKNREQDDIPLITKKAESKSPPKDRKRSKTSQLPQK

SEQ ID NO: 959 - RNA, 36 nt, Artificial Sequence
  Synthetic
guuggaaugacuaauuuuugugcccaccguuggcac

SEQ ID NO: 960 - RNA, 24 nt, Artificial Sequence
  Synthetic
aauuuuugugcccaucguuggcac

SEQ ID NO: 961 - RNA, 23 nt, Artificial Sequence
  Synthetic
auuuuugugcccaucguuggcac

SEQ ID NO: 962 - RNA, 36 nt, Artificial Sequence
  Synthetic
cuagcaaugaccuaauaguguguccuuaguugacau

SEQ ID NO: 963 - RNA, 36 nt, Artificial Sequence
  Synthetic
ccuacaauaccuaagaaauccguccuaaguugacgg

SEQ ID NO: 964 - RNA, 22 nt, Artificial Sequence
  Synthetic
auaguguguccuuaguugacau

SEQ ID NO: 965 - protein, 1093 aa, Artificial Sequence
  Synthetic
MSNKEKNASETRKAYTTKMIPRSHDRMKLLGNFMDYLMDGTPIFFELWNQFGGGIDRDIISGTANKDKISDDLLLAVNWFKVMPINSKPQGVSPSNLANLFQQYSGSEPDIQAQEYFASNFDTEKHQWKDMRVEYERLLAELQLSRSDMHHDLKLMYKEKCIGLSLSTAHYITSVMFGTGAKNNRQTKHQFYSKVIQLLEESTQINSVEQLASIILKAGDCDSYRKLRIRCSRKGATPSILKIVQDYELGTNHDDEVNVPSLIANLKEKLGRFEYECEWKCMEKIKAFLASKVGPYYLGSYSAMLENALSPIKGMTTKNCKFVLKQIDAKNDIKYENEPFGKIVEGFFDSPYFESDTNVKWVLHPHHIGESNIKTLWEDLNAIHSKYEEDIASLSEDKKEKRIKVYQGDVCQTINTYCEEVGKEAKTPLVQLLRYLYSRKDDIAVDKIIDGITFLSKKHKVEKQKINPVIQKYPSFNFGNNSKLLGKIISPKDKLKHNLKCNRNQVDNYIWIEIKVLNTKTMRWEKHHYALSSTRFLEEVYYPATSENPPDALAARFRTKTNGYEGKPALSAEQIEQIRSAPVGLRKVKKRQMRLEAARQQNLLPRYTWGKDFNINICKRGNNFEVTLATKVKKKKEKNYKVVLGYDANIVRKNTYAAIEAHANGDGVIDYNDLPVKPIESGFVTVESQVRDKSYDQLSYNGVKLLYCKPHVESRRSFLEKYRNGTMKDNRGNNIQIDFMKDFEAIADDETSLYYFNMKYCKLLQSSIRNHSSQAKEYREEIFELLRDGKLSVLKLSSLSNLSFVMFKVAKSLIGTYFGHLLKKPKNSKSDVKAPPITDEDKQKADPEMFALRLALEEKRLNKVKSKKEVIANKIVAKALELRDKYGPVLIKGENISDTTKKGKKSSTNSFLMDWLARGVANKVKEMVMMHQGLEFVEVNPNFTSHQDPFVHKNPENTFRARYSRCTPSELTEKNRKEILSFLSDKPSKRPTNAYYNEGAMAFLATYGLKKNDVLGVSLEKFKQIMANILHQRSEDQLLFPSRGGMFYLATYKLDADATSVNWNGKQFWVCNADLVAAYNVGLVDIQKDFKKK

SEQ ID NO: 966 - protein, 1098 aa, Artificial Sequence
  Synthetic
MSISNNNILPYNPKLLPDDRKHKMLVDTFNQLDLIRNNLHDMIIALYGALKYDNIKQFASKEKPHISADALCSINWFRLVKTNERKPAIESNQIISKFIQYSGHTPDKYALSHITGNHEPSHKWIDCREYAINYARIMHLSFSQFQDLATACLNCKILILNGTLTSSWAWGANSALFGGSDKENFSVKAKILNSFIENLKDEMNTTKFQVVEKVCQQIGSSDAADLFDLYRSTVKDGNRGPATGRNPKVMNLFSQDGEISSEQREDFIESFQKVMQEKNSKQIIPHLDKLKYHLVKQSGLYDIYSWAAAIKNANSTIVASNSSNLNTILNKTEKQQTFEELRKDEKIVACSKILLSVNDTLPEDLHYNPSTSNLGKNLDVFFDLLNENSVHTIENKEEKNKIVKECVNQYMEECKGLNKPPMPVLLTFISDYAHKHQAQDFLSAAKMNFIDLKIKSIKVVPTVHGSSPYTWISNLSKKNKDGKMIRTPNSSLIGWIIPPEEIHDQKFAGQNPIIWAVLRVYCNNKWEMHHFPFSDSRFFTEVYAYKPNLPYLPGGENRSKRFGYRHSTNLSNESRQILLDKSKYAKANKSVLRCMENMTHNVVFDPKTSLNIRIKTDKNNSPVLDDKGRITFVMQINHRILEKYNNTKIEIGDRILAYDQNQSENHTYAILQRTEEGSHAHQFNGWYVRVLETGKVTSIVQGLSGPIDQLNYDGMPVTSHKFNCWQADRSAFVSQFASLKISETETFDEAYQAINAQGAYTWNLFYLRILRKALRVCHMENINQFREEILAISKNRLSPMSLGSLSQNSLKMIRAFKSIINCYMSRMSFVDELQKKEGDLELHTIMRLTDNKLNDKRVEKINRASSFLTNKAHSMGCKMIVGESDLPVADSKTSKKQNVDRMDWCARALSHKVEYACKLMGLAYRGIPAYMSSHQDPLVHLVESKRSVLRPRFVVADKSDVKQHHLDNLRRMLNSKTKVGTAVYYREAVELMCEELGIHKTDMAKGKVSLSDFVDKFIGEKAIFPQRGGRFYMSTKRLTTGAKLICYSGSDVWLSDADEIAAINIGMFVVCDQTGAFKKKKKEKLDDEECDILPFRPM

SEQ ID NO: 967 - RNA, 43 nt, Artificial Sequence
  Synthetic
agaaauccgucuuucauugacggcaaagucuauauaugacuau

SEQ ID NO: 968 - RNA, 43 nt, Artificial Sequence
  Synthetic
agaaauccgucuuucauugacggggaaguacugauuuagcaug

SEQ ID NO: 969 - RNA, 43 nt, Artificial Sequence
  Synthetic
agaaauccgucuuucauugacggaucauuugccccagaccugu

SEQ ID NO: 970 - RNA, 43 nt, Artificial Sequence
  Synthetic
agaaauccgucuuucauugacggggcugauaauauugcagcau

SEQ ID NO: 971 - RNA, 43 nt, Artificial Sequence
  Synthetic
agaaauccgucuuucauugacggguaucaaugauuaugaacaa

SEQ ID NO: 972 - RNA, 43 nt, Artificial Sequence
  Synthetic
agaaauccgucuuucauugacgguaucaaugauuaugaacaac

SEQ ID NO: 973 - RNA, 43 nt, Artificial Sequence
  Synthetic
agaaauccgucuuucauugacggugaacaacaugcuaaaucag

SEQ ID NO: 974 - RNA, 43 nt, Artificial Sequence
  Synthetic
agaaauccgucuuucauugacgggcauguuguucauaaucauu

SEQ ID NO: 975 - RNA, 43 nt, Artificial Sequence
  Synthetic
agaaauccgucuuucauugacggagcauguuguucauaaucau

SEQ ID NO: 976 - RNA, 43 nt, Artificial Sequence
  Synthetic
agaaauccgucuuucauugacgggaaguacugauuuagcaugu

SEQ ID NO: 977 - RNA, 43 nt, Artificial Sequence
  Synthetic
agaaauccgucuuucauugacggcaggucuggggcaaaugaug

SEQ ID NO: 978 - RNA, 43 nt, Artificial Sequence
  Synthetic
agaaauccgucuuucauugacggccccagaccuguaauaguca

SEQ ID NO: 979 - RNA, 43 nt, Artificial Sequence
  Synthetic
agaaauccgucuuucauugacgggccccagaccuguaauaguc

SEQ ID NO: 980 - RNA, 43 nt, Artificial Sequence
  Synthetic
agaaauccgucuuucauugacgguucaucauuugccccagacc

SEQ ID NO: 981 - RNA, 43 nt, Artificial Sequence
  Synthetic
agaaauccgucuuucauugacggcuucaucauuugccccagac

SEQ ID NO: 982 - RNA, 43 nt, Artificial Sequence
  Synthetic
agaaauccgucuuucauugacggaaugcugcaauauuaucagc

SEQ ID NO: 983 - RNA, 43 nt, Artificial Sequence
  Synthetic
agaaauccgucuuucauugacggcuuaccuggaaaaugcugca

SEQ ID NO: 984 - RNA, 43 nt, Artificial Sequence
  Synthetic
agaaauccgucuuucauugacggucuuaccuggaaaaugcugc

SEQ ID NO: 985 - RNA, 43 nt, Artificial Sequence
  Synthetic
agaaauccgucuuucauugacgguguuuuaggacagaggguca

SEQ ID NO: 986 - RNA, 43 nt, Artificial Sequence
  Synthetic
agaaauccgucuuucauugacggcuccuaccucucacaguggc

SEQ ID NO: 987 - RNA, 43 nt, Artificial Sequence
  Synthetic
agaaauccgucuuucauugacggauucuagauggaagcuguau

SEQ ID NO: 988 - RNA, 43 nt, Artificial Sequence
  Synthetic
agaaauccgucuuucauugacgguagauggaagcuguauccaa

SEQ ID NO: 989 - RNA, 43 nt, Artificial Sequence
  Synthetic
agaaauccgucuuucauugacggcggagcauccuuggauacag

SEQ ID NO: 990 - RNA, 43 nt, Artificial Sequence
  Synthetic
agaaauccgucuuucauugacggcugaaacagaucugucgacu

SEQ ID NO: 991 - RNA, 43 nt, Artificial Sequence
  Synthetic
agaaauccgucuuucauugacggagcaacauuccggagcaucc

SEQ ID NO: 992 - RNA, 43 nt, Artificial Sequence
  Synthetic
agaaauccgucuuucauugacggcagcaacauuccggagcauc

SEQ ID NO: 993 - RNA, 43 nt, Artificial Sequence
  Synthetic
agaaauccgucuuucauugacgguaggacagagggucagcaug

SEQ ID NO: 994 - RNA, 43 nt, Artificial Sequence
  Synthetic
agaaauccgucuuucauugacggaggacagagggucagcaugc

SEQ ID NO: 995 - RNA, 43 nt, Artificial Sequence
  Synthetic
agaaauccgucuuucauugacggggacagagggucagcaugcc

SEQ ID NO: 996 - RNA, 43 nt, Artificial Sequence
  Synthetic
agaaauccgucuuucauugacggcuuucucagccugucagucc

SEQ ID NO: 997 - RNA, 43 nt, Artificial Sequence
  Synthetic
agaaauccgucuuucauugacggcucagccugucagucccugg

SEQ ID NO: 998 - RNA, 43 nt, Artificial Sequence
  Synthetic
agaaauccgucuuucauugacggccagggacugacaggcugag

SEQ ID NO: 999 - RNA, 43 nt, Artificial Sequence
  Synthetic
agaaauccgucuuucauugacggcugggccaccuccucaauug

SEQ ID NO: 1000 - RNA, 43 nt, Artificial Sequence
  Synthetic
agaaauccgucuuucauugacggaauugaggagguggcccagg

SEQ ID NO: 1001 - RNA, 43 nt, Artificial Sequence
  Synthetic
agaaauccgucuuucauugacgguucaauugaggagguggccc

SEQ ID NO: 1002 - RNA, 43 nt, Artificial Sequence
  Synthetic
agaaauccgucuuucauugacggcgccacuucuucaauugagg

SEQ ID NO: 1003 - RNA, 43 nt, Artificial Sequence
  Synthetic
agaaauccgucuuucauugacggguuggcugcaacuguauauc

SEQ ID NO: 1004 - RNA, 43 nt, Artificial Sequence
  Synthetic
agaaauccgucuuucauugacggucgguccuuguagauauaca

SEQ ID NO: 1005 - RNA, 43 nt, Artificial Sequence
  Synthetic
agaaauccgucuuucauugacggucugccugccgcacuagcuu

SEQ ID NO: 1006 - RNA, 43 nt, Artificial Sequence
  Synthetic
agaaauccgucuuucauugacgguuucucagccugucaguccc

SEQ ID NO: 1007 - RNA, 43 nt, Artificial Sequence
  Synthetic
agaaauccgucuuucauugacggucagccugucagucccuggg

SEQ ID NO: 1008 - RNA, 43 nt, Artificial Sequence
  Synthetic
agaaauccgucuuucauugacggaguuccugggccaccuccuc

SEQ ID NO: 1009 - RNA, 43 nt, Artificial Sequence
  Synthetic
agaaauccgucuuucauugacggaggagguggcccaggaacuc

SEQ ID NO: 1010 - RNA, 43 nt, Artificial Sequence
  Synthetic
agaaauccgucuuucauugacggaagaaguggcggaagcuggu

SEQ ID NO: 1011 - RNA, 43 nt, Artificial Sequence
  Synthetic
agaaauccgucuuucauugacgggcugcaacuguauaucuaca

SEQ ID NO: 1012 - RNA, 43 nt, Artificial Sequence
  Synthetic
agaaauccgucuuucauugacggcagccaacgaagugccucag

SEQ ID NO: 1013 - RNA, 43 nt, Artificial Sequence
  Synthetic
agaaauccgucuuucauugacgguagauauacaguugcagcca

SEQ ID NO: 1014 - RNA, 43 nt, Artificial Sequence
  Synthetic
agaaauccgucuuucauugacgggugacuucucgguccuugua

SEQ ID NO: 1015 - RNA, 43 nt, Artificial Sequence
  Synthetic
agaaauccgucuuucauugacgggugacaguggacacaccuua

SEQ ID NO: 1016 - RNA, 43 nt, Artificial Sequence
  Synthetic
agaaauccgucuuucauugacggugacaguggacacaccuuac

SEQ ID NO: 1017 - RNA, 43 nt, Artificial Sequence
  Synthetic
agaaauccgucuuucauugacggccugggcaaccgucuggaug

SEQ ID NO: 1018 - RNA, 43 nt, Artificial Sequence
  Synthetic
agaaauccgucuuucauugacggcccagguaagguguguccac

SEQ ID NO: 1019 - RNA, 43 nt, Artificial Sequence
  Synthetic
agaaauccgucuuucauugacggcgcacaucauccagacgguu

SEQ ID NO: 1020 - RNA, 43 nt, Artificial Sequence
  Synthetic
agaaauccgucuuucauugacggaaucuguuacgcacaucauc

SEQ ID NO: 1021 - RNA, 43 nt, Artificial Sequence
  Synthetic
agaaauccgucuuucauugacgggaaucuguuacgcacaucau

SEQ ID NO: 1022 - RNA, 43 nt, Artificial Sequence
  Synthetic
agaaauccgucuuucauugacgguggcggcaguuugaaucugu

SEQ ID NO: 1023 - RNA, 43 nt, Artificial Sequence
  Synthetic
agaaauccgucuuucauugacggccugaguuguggcggcaguu

SEQ ID NO: 1024 - DNA, 1113 bp, Artificial Sequence
  Synthetic
atgctcccccggctaatttgtatcaatgattatgaacaacatgctaaatcagtacttccaaagtctatatatgactattacaggtctggggcaaatgatgaagaaactttggctgataatattgcagcattttccagatggaagctgtatccaaggatgctccggaatgttgctgaaacagatctgtcgacttctgttttaggacagagggtcagcatgccaatatgtgtgggggctacggccatgcagcgcatggctcatgtggacggcgagcttgccactgtgagagcctgtcagtccctgggaacgggcatgatgttgagttcctgggccacctcctcaattgaagaagtggcggaagctggtcctgaggcacttcgttggctgcaactgtatatctacaaggaccgagaagtcaccaagaagctagtgcggcaggcagagaagatgggctacaaggccatatttgtgacagtggacacaccttacctgggcaaccgtctggatgatgtgcgtaacagattcaaactgccgccacaactcaggatgaaaaattttgaaaccagtactttatcattttctcctgaggaaaattttggagacgacagtggacttgctgcatatgtggctaaagcaatagacccatctatcagctgggaagatatcaaatggctgagaagactgacatcattgccaattgttgcaaagggcattttgagaggtgatgatgccagggaggctgttaaacatggcttgaatgggatcttggtgtcgaatcatggggctcgacaactcgatggggtgccagccactattgatgttctgccagaaattgtggaggctgtggaagggaaggtggaagtcttcctggacgggggtgtgcggaaaggcactgatgttctgaaagctctggctcttggcgccaaggctgtgtttgtggggagaccaatcgtttggggcttagctttccagggggagaaaggtgttcaagatgtcctcgagatactaaaggaagaattccggttggccatggctctgagtgggtgccagaatgtgaaagtcatcgacaagacattggtgaggaaaaatcctttggccgtttccaagatctga

SEQ ID NO: 1025 - DNA, 20 bp, Artificial Sequence
  Synthetic
caaagtctatatatgactat

SEQ ID NO: 1026 - DNA, 20 bp, Artificial Sequence
  Synthetic
ggaagtactgatttagcatg

SEQ ID NO: 1027 - DNA, 20 bp, Artificial Sequence
  Synthetic
atcatttgccccagacctgt

SEQ ID NO: 1028 - DNA, 20 bp, Artificial Sequence
  Synthetic
ggctgataatattgcagcat

SEQ ID NO: 1029 - DNA, 20 bp, Artificial Sequence
  Synthetic
gtatcaatgattatgaacaa

SEQ ID NO: 1030 - DNA, 20 bp, Artificial Sequence
  Synthetic
tatcaatgattatgaacaac

SEQ ID NO: 1031 - DNA, 20 bp, Artificial Sequence
  Synthetic
tgaacaacatgctaaatcag

SEQ ID NO: 1032 - DNA, 20 bp, Artificial Sequence
  Synthetic
gcatgttgttcataatcatt

SEQ ID NO: 1033 - DNA, 20 bp, Artificial Sequence
  Synthetic
agcatgttgttcataatcat

SEQ ID NO: 1034 - DNA, 20 bp, Artificial Sequence
  Synthetic
gaagtactgatttagcatgt

SEQ ID NO: 1035 - DNA, 20 bp, Artificial Sequence
  Synthetic
caggtctggggcaaatgatg

SEQ ID NO: 1036 - DNA, 20 bp, Artificial Sequence
  Synthetic
ccccagacctgtaatagtca

SEQ ID NO: 1037 - DNA, 20 bp, Artificial Sequence
  Synthetic
gccccagacctgtaatagtc

SEQ ID NO: 1038 - DNA, 20 bp, Artificial Sequence
  Synthetic
ttcatcatttgccccagacc

SEQ ID NO: 1039 - DNA, 20 bp, Artificial Sequence
  Synthetic
cttcatcatttgccccagac

SEQ ID NO: 1040 - DNA, 20 bp, Artificial Sequence
  Synthetic
aatgctgcaatattatcagc

SEQ ID NO: 1041 - DNA, 20 bp, Artificial Sequence
  Synthetic
cttacctggaaaatgctgca

SEQ ID NO: 1042 - DNA, 20 bp, Artificial Sequence
  Synthetic
tcttacctggaaaatgctgc

SEQ ID NO: 1043 - DNA, 20 bp, Artificial Sequence
  Synthetic
tgttttaggacagagggtca

SEQ ID NO: 1044 - DNA, 20 bp, Artificial Sequence
  Synthetic
ctcctacctctcacagtggc

SEQ ID NO: 1045 - DNA, 20 bp, Artificial Sequence
  Synthetic
attctagatggaagctgtat

SEQ ID NO: 1046 - DNA, 20 bp, Artificial Sequence
  Synthetic
tagatggaagctgtatccaa

SEQ ID NO: 1047 - DNA, 20 bp, Artificial Sequence
  Synthetic
cggagcatccttggatacag

SEQ ID NO: 1048 - DNA, 20 bp, Artificial Sequence
  Synthetic
ctgaaacagatctgtcgact

SEQ ID NO: 1049 - DNA, 20 bp, Artificial Sequence
  Synthetic
agcaacattccggagcatcc

SEQ ID NO: 1050 - DNA, 20 bp, Artificial Sequence
  Synthetic
cagcaacattccggagcatc

SEQ ID NO: 1051 - DNA, 20 bp, Artificial Sequence
  Synthetic
taggacagagggtcagcatg

SEQ ID NO: 1052 - DNA, 20 bp, Artificial Sequence
  Synthetic
aggacagagggtcagcatgc

SEQ ID NO: 1053 - DNA, 20 bp, Artificial Sequence
  Synthetic
ggacagagggtcagcatgcc

SEQ ID NO: 1054 - DNA, 20 bp, Artificial Sequence
  Synthetic
ctttctcagcctgtcagtcc

SEQ ID NO: 1055 - DNA, 20 bp, Artificial Sequence
  Synthetic
ctcagcctgtcagtccctgg

SEQ ID NO: 1056 - DNA, 20 bp, Artificial Sequence
  Synthetic
ccagggactgacaggctgag

SEQ ID NO: 1057 - DNA, 20 bp, Artificial Sequence
  Synthetic
ctgggccacctcctcaattg

SEQ ID NO: 1058 - DNA, 20 bp, Artificial Sequence
  Synthetic
aattgaggaggtggcccagg

SEQ ID NO: 1059 - DNA, 20 bp, Artificial Sequence
  Synthetic
ttcaattgaggaggtggccc

SEQ ID NO: 1060 - DNA, 20 bp, Artificial Sequence
  Synthetic
cgccacttcttcaattgagg

SEQ ID NO: 1061 - DNA, 20 bp, Artificial Sequence
  Synthetic
gttggctgcaactgtatatc

SEQ ID NO: 1062 - DNA, 20 bp, Artificial Sequence
  Synthetic
tcggtccttgtagatataca

SEQ ID NO: 1063 - DNA, 20 bp, Artificial Sequence
  Synthetic
tctgcctgccgcactagctt

SEQ ID NO: 1064 - DNA, 20 bp, Artificial Sequence
  Synthetic
tttctcagcctgtcagtccc

SEQ ID NO: 1065 - DNA, 20 bp, Artificial Sequence
  Synthetic
tcagcctgtcagtccctggg

SEQ ID NO: 1066 - DNA, 20 bp, Artificial Sequence
  Synthetic
agttcctgggccacctcctc

SEQ ID NO: 1067 - DNA, 20 bp, Artificial Sequence
  Synthetic
aggaggtggcccaggaactc

SEQ ID NO: 1068 - DNA, 20 bp, Artificial Sequence
  Synthetic
aagaagtggcggaagctggt

SEQ ID NO: 1069 - DNA, 20 bp, Artificial Sequence
  Synthetic
gctgcaactgtatatctaca

SEQ ID NO: 1070 - DNA, 20 bp, Artificial Sequence
  Synthetic
cagccaacgaagtgcctcag

SEQ ID NO: 1071 - DNA, 20 bp, Artificial Sequence
  Synthetic
tagatatacagttgcagcca

SEQ ID NO: 1072 - DNA, 20 bp, Artificial Sequence
  Synthetic
gtgacttctcggtccttgta

SEQ ID NO: 1073 - DNA, 20 bp, Artificial Sequence
  Synthetic
gtgacagtggacacacctta

SEQ ID NO: 1074 - DNA, 20 bp, Artificial Sequence
  Synthetic
tgacagtggacacaccttac

SEQ ID NO: 1075 - DNA, 20 bp, Artificial Sequence
  Synthetic
cctgggcaaccgtctggatg

SEQ ID NO: 1076 - DNA, 20 bp, Artificial Sequence
  Synthetic
cccaggtaaggtgtgtccac

SEQ ID NO: 1077 - DNA, 20 bp, Artificial Sequence
  Synthetic
cgcacatcatccagacggtt

SEQ ID NO: 1078 - DNA, 20 bp, Artificial Sequence
  Synthetic
aatctgttacgcacatcatc

SEQ ID NO: 1079 - DNA, 20 bp, Artificial Sequence
  Synthetic
gaatctgttacgcacatcat

SEQ ID NO: 1080 - DNA, 20 bp, Artificial Sequence
  Synthetic
tggcggcagtttgaatctgt

SEQ ID NO: 1081 - DNA, 20 bp, Artificial Sequence
  Synthetic
cctgagttgtggcggcagtt

SEQ ID NO: 1082 - RNA, 43 nt, Artificial Sequence; identical to SEQ ID NO: 989
  Synthetic
  misc_feature (40)..(43): modified with phosphorothioate
  misc_feature (40)..(42): modified with 2′-O-methlyation

SEQ ID NO: 1083 - RNA, 43 nt, Artificial Sequence; identical to SEQ ID NO: 989
  Synthetic
  misc_feature (1)..(4): modified with phosphorothioate
  misc_feature (1)..(3): modified with 2′-O-methlyation
  misc_feature (40)..(42): modified with 2′-O-methlyation
  misc_feature (40)..(43): modified with phosphorothioate

SEQ ID NO: 1084 - RNA, 43 nt, Artificial Sequence; identical to SEQ ID NO: 968
  Synthetic
  misc_feature (40)..(43): modified with phosphorothioate
  misc_feature (40)..(42): modified with 2′-O-methlyation

SEQ ID NO: 1085 - RNA, 43 nt, Artificial Sequence; identical to SEQ ID NO: 968
  Synthetic
  misc_feature (1)..(4): modified with phosphorothioate
  misc_feature (1)..(3): modified with 2′-O-methlyation
  misc_feature (40)..(42): modified with 2′-O-methlyation
  misc_feature (40)..(43): modified with phosphorothioate

SEQ ID NO: 1086 - RNA, 43 nt, Artificial Sequence; identical to SEQ ID NO: 967
  Synthetic
  misc_feature (40)..(43): modified with phosphorothioate
  misc_feature (40)..(42): modified with 2′-O-methlyation

SEQ ID NO: 1087 - RNA, 43 nt, Artificial Sequence; identical to SEQ ID NO: 967
  Synthetic
  misc_feature (1)..(4): modified with phosphorothioate
  misc_feature (1)..(3): modified with 2′-O-methlyation
  misc_feature (40)..(42): modified with 2′-O-methlyation
  misc_feature (40)..(43): modified with phosphorothioate

SEQ ID NO: 1088 - DNA, 122 bp, Artificial Sequence
  Synthetic
attgtgcactgtcagatcttggaaacggccaaaggatttttcctcaccaatgtcttgtcgatgactttcacattctggcacccactcagagccatggccaaccggaattcttcctttagtat

SEQ ID NO: 1089 - RNA, 3213 nt, Artificial Sequence
  Synthetic
augagcuccgccaucaaguccuacaagucugugcugcggccaaacgagagaaagaaucagcugcugaaguccaccauccagugccuggaggacggcuccgccuucuuuuucaagaugcugcagggccuguuuggcggcaucacccccgagaucgugagauucagcacagagcaggagaagcagcagcaggauaucgcccuguggugugccgugaauugguucaggccugugagccaggacucccugacccacacaaucgccuccgauaaccugguggagaaguuugaggaguacuauggcggcacagccagcgacgccaucaagcaguacuucagcgccuccaucggcgaguccuacuauuggaaugacugccgccagcaguacuaugaucugugucgggagcugggcguggaggugucugaccugacccacgaucuggagauccugugccgggagaagugucuggccguggccacagagagcaaccagaacaauucuaucaucagcgugcuguuuggcaccggcgagaaggaggauaggucugugaagcugcgcaucacaaagaagauccuggaggccaucagcaaccugaaggagaucccaaagaauguggcccccauccaggagaucauccugaauguggccaaggccaccaaggagacauucagacagguguacgcaggaaaccugggagcaccauccacccuggagaaguuuaucgccaaggacggccagaaggaguucgaucugaagaagcugcagacagaccugaagaaagugauccggggcaagucuaaggagagagauuggugcugucaggaggagcugaggagcuacguggagcagaauaccauccaguaugaccugugggccuggggcgagauguucaacaaggcccacaccgcccugaagaucaaguccacaagaaacuacaauuuugccaagcagaggcuggagcaguucaaggagauccagucucugaacaaucugcugguggugaagaagcugaacgacuuuuucgauagcgaguuuuucuccggcgaggagaccuacacaaucugcgugcaccaccugggcggcaaggaccuguccaagcuguauaaggccugggaggacgaucccgccgauccugagaaugccaucguggugcugugcgacgaucugaagaacaauuuuaagaaggagccuaucaggaacauccugcgcuacaucuucaccauccgccaggaguguagcgcacaggacauccuggcagcagcaaaguacaaucagcagcuggaucgguauaagagccagaaggccaacccauccgugcugggcaaucagggcuuuaccuggacaaacgccgugauccugccagagaaggcccagcggaacgacagacccaauucucuggaucugcgcaucuggcuguaccugaagcugcggcacccugacggcagauggaagaagcaccacaucccauucuacgauacccgguuuuuccaggagaucuaugccgccggcaauagcccuguggacaccugucaguuuaggacaccccgcuucggcuaucaccugccuaagcugaccgaucagacagccauccgcgugaacaagaagcacgugaaggcagcaaagaccgaggcacggaucagacuggccauccagcagggcacacugccaguguccaaucugaagaucaccgagaucuccgccacaaucaacucuaagggccaggugcgcauccccgugaaguuucgggugggaaggcagaagggaacccugcagaucggcgaccgguucugcggcuacgaucagaaccagacagccucucacgccuauagccugugggagguggugaaggagggccaguaccacaaggagcugggcuguuuugugcgcuucaucucuagcggcgacaucguguccaucaccgagaaccggggcaaucaguuugaucagcugucuuaugagggccuggccuacccccaguaugccgacuggagaaagaaggccuccaaguucgugucucuguggcagaucaccaagaagaacaagaagaaggagaucgugacaguggaggccaaggagaaguuugacgccaucugcaaguaccagccuaggcuguauaaguucaacaaggaguacgccuaucugcugcgggauaucgugagaggcaagagccugguggagcugcagcagaucaggcaggagaucuuucgcuucaucgagcaggacuguggagugacccgccugggaucucugagccuguccacccuggagacagugaaggccgugaagggcaucaucuacuccuauuuuucuacagcccugaaugccucuaagaacaaucccaucagcgacgagcagcggaaggaguuugauccugagcuguucgcccugcuggagaagcuggagcugaucaggacucggaagaagaagcagaagguggagagaaucgccaauagccugauccagacaugccuggagaacaauaucaaguucaucaggggcgagggcgaccuguccaccacaaacaaugccaccaagaagaaggccaacucuaggagcauggauuggcuggccagaggcguguuuaauaagauccggcagcuggccccaaugcacaacaucacccuguucggcugcggcagccuguacacaucccaccaggacccucuggugcacagaaacccagauaaggccaugaaguguagaugggcagcaaucccagugaaggacaucggcgauugggugcugagaaagcugucccagaaccugagggccaagaaucggggcaccggcgaguacuaucaccagggcgugaaggaguuccugucucacuaugagcugcaggaccuggaggaggagcugcugaaguggcggucugauagaaagagcaacaucccuugcugggugcugcagaauagacuggccgagaagcugggcaacaaggaggccgugguguacaucccagugaggggcggccgcaucuauuuugcaacccacaagguggcaacaggagccgugagcaucguguucgaccagaagcaaguguggguguguaaugcagaucacguggcagcagcaaacaucgcacugaccggcaagggcaucggcgagcaguccucugacgaggagaaccccgauggcuccaggaucaagcugcagcugacaucuaaaaggccggcggccacgaaaaaggccggccaggcaaaaaagaaaaaguaa

SEQ ID NO: 1090 - RNA, 3213 nt, Artificial Sequence
  Synthetic
augagcuccgccaucaaguccuacaagucugugcugcggccaaacgagagaaagaaucagcugcugaaguccaccauccagugccuggaggacggcuccgccuucuuuuucaagaugcugcagggccuguuuggcggcaucacccccgagaucgugagauucagcacagagcaggagaagcagcagcaggauaucgcccuguggugugccgugaauugguucaggccugugagccaggacucccugacccacacaaucgccuccgauaaccugguggagaaguuugaggaguacuauggcggcacagccagcgacgccaucaagcaguacuucagcgccuccaucggcgaguccuacuauuggaaugacugccgccagcaguacuaugaucugugucgggagcugggcguggaggugucugaccugacccacgaucuggagauccugugccgggagaagugucuggccguggccacagagagcaaccagaacaauucuaucaucagcgugcuguuuggcaccggcgagaaggaggauaggucugugaagcugcgcaucacaaagaagauccuggaggccaucagcaaccugaaggagaucccaaagaauguggcccccauccaggagaucauccugaauguggccaaggccaccaaggagacauucagacagguguacgcaggaaaccugggagcaccauccacccuggagaaguuuaucgccaaggacggccagaaggaguucgaucugaagaagcugcagacagaccugaagaaagugauccggggcaagucuaaggagagagauuggugcugucaggaggagcugaggagcuacguggagcagaauaccauccaguaugaccugugggccuggggcgagauguucaacaaggcccacaccgcccugaagaucaaguccacaagaaacuacaauuuugccaagcagaggcuggagcaguucaaggagauccagucucugaacaaucugcugguggugaagaagcugaacgacuuuuucgauagcgaguuuuucuccggcgaggagaccuacacaaucugcgugcaccaccugggcggcaaggaccuguccaagcuguauaaggccugggaggacgaucccgccgauccugagaaugccaucguggugcugugcgacgaucugaagaacaauuuuaagaaggagccuaucaggaacauccugcgcuacaucuucaccauccgccaggaguguagcgcacaggacauccuggcagcagcaaaguacaaucagcagcuggaucgguauaagagccagaaggccaacccauccgugcugggcaaucagggcuuuaccuggacaaacgccgugauccugccagagaaggcccagcggaacgacagacccaauucucuggaucugcgcaucuggcuguaccugaagcugcggcacccugacggcagauggaagaagcaccacaucccauucuacgauacccgguuuuuccaggagaucuaugccgccggcaauagcccuguggacaccugucaguuuaggacaccccgcuucggcuaucaccugccuaagcugaccgaucagacagccauccgcgugaacaagaagcacgugaaggcagcaaagaccgaggcacggaucagacuggccauccagcagggcacacugccaguguccaaucugaagaucaccgagaucuccgccacaaucaacucuaagggccaggugcgcauccccgugaaguuucgggugggaaggcagaagggaacccugcagaucggcgaccgguucugcggcuacgaucagaaccagacagccucucacgccuauagccugugggagguggugaaggagggccaguaccacaaggagcugcggugucgggugcgcuucaucucuagcggcgacaucguguccaucaccgagaaccggggcaaucaguuugaucagcugucuuaugagggccuggccuacccccaguaugccgacuggagaaagaaggccuccaaguucgugucucuguggcagaucaccaagaagaacaagaagaaggagaucgugacaguggaggccaaggagaaguuugacgccaucugcaaguaccagccuaggcuguauaaguucaacaaggaguacgccuaucugcugcgggauaucgugagaggcaagagccugguggagcugcagcagaucaggcaggagaucuuucgcuucaucgagcaggacuguggagugacccgccugggaucucugagccuguccacccuggagacagugaaggccgugaagggcaucaucuacuccuauuuuucuacagcccugaaugccucuaagaacaaucccaucagcgacgagcagcggaaggaguuugauccugagcuguucgcccugcuggagaagcuggagcugaucaggacucggaagaagaagcagaagguggagagaaucgccaauagccugauccagacaugccuggagaacaauaucaaguucaucaggggcgagggcgaccuguccaccacaaacaaugccaccaagaagaaggccaacucuaggagcauggauuggcuggccagaggcguguuuaauaagauccggcagcuggccaccaugcacaacaucacccuguucggcugcggcagccuguacacaucccaccaggacccucuggugcacagaaacccagauaaggccaugaaguguagaugggcagcaaucccagugaaggacaucggcgauugggugcugagaaagcugucccagaaccugagggccaagaaucggggcaccggcgaguacuaucaccagggcgugaaggaguuccugucucacuaugagcugcaggaccuggaggaggagcugcugaaguggcggucugauagaaagagcaacaucccuugcugggugcugcagaauagacuggccgagaagcugggcaacaaggaggccgugguguacaucccagugaggggcggccgcaucuauuuugcaacccacaagguggcaacaggagccgugagcaucguguucgaccagaagcaaguguggguguguaaugcagaucacguggcagcagcaaacaucgcacugaccggcaagggcaucggccggcaguccucugacgaggagaaccccgauggcggcaggaucaagcugcagcugacaucuaaaaggccggcggccacgaaaaaggccggccaggcaaaaaagaaaaaguaa

SEQ ID NO: 1091 - RNA, 43 nt, Artificial Sequence; identical to SEQ ID NO: 989
  Synthetic
  misc_feature (40)..(43): modified with phosphorothioate
  misc_feature (40)..(42): modified with 2′-O-methlyation

SEQ ID NO: 1092 - RNA, 43 nt, Artificial Sequence; identical to SEQ ID NO: 989
  Synthetic
  misc_feature (1)..(4): modified with phosphorothioate
  misc_feature (1)..(3): modified with 2′-O-methlyation
  misc_feature (40)..(42): modified with 2′-O-methlyation
  misc_feature (40)..(43): modified with phosphorothioate

SEQ ID NO: 1093 - RNA, 20 nt, Artificial Sequence
  Synthetic
caaagucuauauaugacuau

SEQ ID NO: 1094 - RNA, 20 nt, Artificial Sequence
  Synthetic
ggaaguacugauuuagcaug

SEQ ID NO: 1095 - RNA, 20 nt, Artificial Sequence
  Synthetic
uagauggaagcuguauccaa

SEQ ID NO: 1096 - RNA, 20 nt, Artificial Sequence
  Synthetic
cggagcauccuuggauacag

SEQ ID NO: 1097 - RNA, 20 nt, Artificial Sequence
  Synthetic
aggacagagggucagcaugc